(19) United States
(12) Patent Application Publication
(10) Pub. No.: US 2019/0290686 A1
(43) Pub. Date: Sep. 26, 2019

(54) ARTIFICIAL ANTIGEN PRESENTING CELLS AND METHODS OF USE

(71) Applicant: Rubius Therapeutics, Inc., Cambridge, MA (US)

(72) Inventors: Thomas Joseph Wickham, Groton, MA (US); Tiffany Fen-yi Chen, Cambridge, MA (US); Sivan Elloul, Newton, MA (US); Regina Sophia Salvat, Cambridge, MA (US); Nathan J. Dowden, Winchester, MA (US)

(73) Assignee: Rubius Therapeutics, Inc., Cambridge, MA (US)

(21) Appl. No.: 16/231,489

(22) Filed: Dec. 22, 2018

Related U.S. Application Data
(60) Provisional application No. 62/610,149, filed on Dec. 23, 2017.
(60) Provisional application No. 62/650,250, filed on Mar. 29, 2018.
(60) Provisional application No. 62/665,445, filed on May 1, 2018.
(60) Provisional application No. 62/680,544, filed on Jun. 4, 2018.
(60) Provisional application No. 62/686,656, filed on Jun. 18, 2018.
(60) Provisional application No. 62/688,324, filed on Jun. 21, 2018.
(60) Provisional application No. 62/692,623, filed on Jun. 29, 2018.
(60) Provisional application No. 62/745,253, filed on Oct. 12, 2018.
(60) Provisional application No. 62/757,741, filed on Nov. 8, 2018.

(51) Int. Cl. A61K 35/15; A61K 35/18; A61K 35/19; C12N 5/0783; C12N 5/078

(52) U.S. Cl. CPC A61K 35/15; A61K 35/18; C12N 5/0644; C12N 5/0637; C12N 5/0641; A61K 35/19

(57) ABSTRACT

The present disclosure relates to artificial antigen presenting cells (aAPCs), in particular engineered erythroid cells and enucleated cells (e.g. enucleated erythroid cells and platelets), that are engineered to activate or suppress T cells.

### RELATED APPLICATIONS

This application claims priority to U.S. Provisional Patent Application No. 62/610,149, filed on Dec. 23, 2017, U.S. Provisional Patent Application No. 62/650,250, filed on Mar. 29, 2018, U.S. Provisional Patent Application No. 62/665,445, filed on May 1, 2018, U.S. Provisional Patent Application No. 62/680,544, filed on Jun. 4, 2018, U.S. Provisional Patent Application No. 62/686,656, filed on Jun. 18, 2018, U.S. Provisional Patent Application No. 62/688,324, filed on Jun. 21, 2018, U.S. Provisional Patent Application No. 62/692,623, filed on Jun. 29, 2018, U.S. Provisional Patent Application No. 62/745,253, filed on Oct. 12, 2018 and U.S. Provisional Patent Application No. 62/757,741, filed on Nov. 8, 2018, the entire contents of each of which are incorporated herein by reference for all purposes.

### SEQUENCE LISTING

The instant application contains a Sequence Listing which has been submitted electronically in ASCII format and is hereby incorporated by reference in its entirety. Said ASCII copy, created on Dec. 21, 2018, is named 129267-00120_SL.txt and is 588,687 bytes in size.

### BACKGROUND

Active immune responses depend on efficient presentation of antigens and co-stimulatory signals by antigen-presenting cells (APCs). Upon internalization of an antigen, the APCs can display antigen-class I and II major histocompatibility complex (MHC) on the membrane together with co-stimulatory signals to activate antigen-specific T cells, which play a key role in the adaptive immune response. In vivo, induction of T cell responses is highly dependent on interactions with professional antigen-presenting cells (APCs), in particular dendritic cells (DCs), which present, for example, tumor-specific antigens. Generally, antigen-specific T cells can be primed and amplified ex vivo before they are transferred back to the patient. For example, in adoptive cell transfer (ACT), tumor-specific T cells are isolated then expanded ex vivo to obtain a large number of cells for transfusion. As one of the APCs, dendritic cells (DCs) are usually used to maximize T cell stimulation ex vivo. However, the use of natural APCs, such as DCs, has been met with certain challenges, including lack of knowledge of the optimal antigen-loaded DC, and mixed results have been found in clinical trials (Steenblock E. R. et al., Expert Opin. Biol. Ther. 2009; 9: 451-464; Melief C M J Immunity. 2008; 29: 372-383; Palucka K. and Banchereau J. Immunity. 2013; 39: 38-48). In addition, isolation and ex vivo stimulation of autologous DCs is time-consuming and expensive, and the quality of ex vivo-generated DCs can be variable (Steenblock E. R. et al. 2009; Kim J. V. et al. Nat. Biotechnol. 2004; 22: 403-410). The use of patient-derived autologous DCs therefore limits standardization of DC-based treatment protocols (Steenblock E. R. et al. 2009; Kim J. V. et al. 2004).

Artificial APCs (aAPCs) are engineered platforms for T cell activation and expansion that aim to avoid the aforementioned obstacles while mimicking the interaction between DCs and T cells. They include multiple systems, including synthetic biomaterials that have been engineered to activate and/or expand desirable immune cell populations (e.g., T cells). These systems may act by mimicking the interaction between DCs and T cells. For instance, several cell-sized, rigid, beads, such as latex microbeads, polystyrene-coated magnetic microbeads and biodegradable poly(lactic-co-glycolic acid) microparticles, have been developed. The efficacy of these beads in inducing activation and/or expansion of immune cells appears to be highly dependent on the properties of the materials used. For example, beads greater than 200 nm are typically retained at the site of inoculation, while smaller particles may be taken up by DCs (see, e.g., Reddy et al. (2006) J. Control. Release 112: 26-34). In contrast, the membrane of natural APCs is much more dynamic than the outer surface of these beads.

There remains a need to provide improved ways to stimulate T cells and to promulgate sufficient numbers of therapeutic T cells for adoptive immunotherapy. The present invention provides novel and inventive red cell therapeutics (RCTs), specifically aAPCs to mimic the functions of APCs, such as dendritic cells (DCs), to stimulate T cells and induce, for example, antitumor or infectious disease immune responses, or to suppress T cell activity to prevent, for example, autoimmune disorders.

### SUMMARY OF THE INVENTION

The present disclosure relates to artificial antigen presenting cells (aAPCs), in particular erythroid cells and enucleated cells (e.g. enucleated erythroid cells and platelets), that are engineered to activate or suppress T cells. The engineered erythroid cells can be nucleated, e.g., erythrocyte precursor cells. The engineered erythroid cells can also be enucleated erythroid cells, e.g., reticulocytes or erythrocytes.

The aAPCs described herein offer numerous advantages over the use of spherical nanoparticles, such as rigid, bead-based aAPCs. As merely one example, the outer surface of a nanoparticle is rigid and immobile, and therefore limits the movement of the polypeptides on its surface, while the outer membrane of an aAPC as described herein (i.e., an erythroid cell or enucleated cell) is dynamic and fluid. An aAPC of the present disclosure therefore allows for greater molecular mobility and more efficient molecular reorganization as compared to nanoparticles, which is highly advantageous for immunological synapse formation and T cell stimulation.

Accordingly, in one aspect, the disclosure provides an artificial antigen presenting cell (aAPC) engineered to activate T cells, wherein the aAPC comprises an enucleated cell, wherein the enucleated cell comprises on the cell surface at least one exogenous antigenic polypeptide disclosed in Table 1 or Tables 14, 15, and 20-24. In some embodiments, the at least one exogenous antigenic polypeptide is a tumor antigen, an autoimmune disease antigen, a viral antigen, a bacterial antigen or a parasite. In some embodiments, the at least one exogenous antigenic polypeptide is selected from the group consisting of: a melanoma antigen genes-A (MAGE-A) antigen, a neutrophil granule protease antigen, a NY-ESO-1/LAGE-2 antigen, a telomerase antigen, a glycoprotein 100 (gp100) antigen, an epstein barr virus (EBV) antigen, a human papilloma virus (HPV) antigen, and a hepatitis B virus (HBV) antigen.

In one aspect, the disclosure provides an artificial antigen presenting cell (aAPC) engineered to activate T cells, wherein the aAPC comprises an enucleated cell, wherein the enucleated cell comprises on the cell surface a first exogenous antigenic polypeptide and a second exogenous antigenic polypeptide, and wherein the first exogenous antigenic polypeptide and the second exogenous antigenic polypeptide have amino acid sequences which overlap by at least 2 amino acids. In some embodiments, the overlap is between 2 amino acids and 23 amino acids.

In some embodiments, the first exogenous antigenic polypeptide, the second exogenous polypeptide, or the first and the second exogenous antigenic polypeptide is a tumor antigen, an autoimmune disease antigen, a viral antigen, a bacterial antigen or a parasite. In some embodiments, the first exogenous antigenic polypeptide, the second exogenous polypeptide, or the first and the second exogenous antigenic polypeptide is a polypeptide disclosed in Table 1 or Tables 14, 15 and 20-24. In some embodiments, the first exogenous antigenic polypeptide, the second exogenous polypeptide, or the first and the second exogenous antigenic polypeptide is selected from the group consisting of: melanoma antigen genes-A (MAGE-A) antigens, neutrophil granule protease antigens, NY-ESO-1/LAGE-2 antigens, telomerase antigens, glycoprotein 100 (gp100) antigens, epstein barr virus (EBV) antigens, human papilloma virus (HPV) antigens, and hepatitis B virus (HBV) antigens.

In some embodiments, the aAPC further comprises on the cell surface an exogenous antigen-presenting polypeptide. In some embodiments, the exogenous antigen-presenting polypeptide is an MHC class I polypeptide, an MHC class I single chain fusion protein, an MHC class II polypeptide, or an MHC class II single chain fusion protein. In some embodiments, the MHC class I polypeptide is selected from the group consisting of: HLA A, HLA B, and HLA C. In some embodiments, the MHC class II polypeptide is selected from the group consisting of: HLA-DPα, HLA-DPβ, HLA-DM, HLA DOA, HLA DOB, HLA DQα, HLA DQβ, HLA DRα, and HLA DRβ.

In another aspect, the disclosure provides an artificial antigen presenting cell (aAPC) engineered to activate T cells, wherein the aAPC comprises an enucleated cell, wherein the enucleated cell comprises on the cell surface an exogenous antigen-presenting polypeptide and an exogenous antigenic polypeptide, wherein the exogenous antigen-presenting polypeptide is an MHC class I single chain fusion protein or an MHC class II single chain fusion protein.

In some embodiments, the MHC class I single chain fusion protein comprises an α-chain, and a β2m chain. In some embodiments, the MHC class I single chain fusion protein further comprises a membrane anchor. In some embodiments, the exogenous antigenic polypeptide is connected to the MHC I single chain fusion protein via a linker. In some embodiments, the linker is a cleavable linker. In some embodiments, the MHC class II single chain fusion protein comprises an α-chain, and a β chain. In some embodiments, the MHC class II single chain fusion protein further comprises a membrane anchor. In some embodiments, the exogenous antigenic polypeptide is connected to the MHC II single chain fusion protein via a linker. In some embodiments, the linker is a cleavable linker. In some embodiments, the anchor comprises a glycophorin A (GPA) protein or a transmembrane domain of small integral membrane protein 1 (SMIM1). In some embodiments, the exogenous antigenic polypeptide is bound to the exogenous antigen-presenting polypeptide covalently. In some embodiments, the exogenous antigenic polypeptide is bound to the exogenous antigen-presenting polypeptide non-covalently.

In some embodiments, the aAPC further comprising on the cell surface at least one exogenous costimulatory polypeptide. In some embodiments, the at least one exogenous costimulatory polypeptide is selected from the group consisting of 4-1BBL, LIGHT, anti CD28, CD80, CD86, CD70, OX40L, GITRL, TIM4, SLAM, CD48, CD58, CD83, CD155, CD112, IL-15Rα fused to IL-15, IL-21, ICAM-1, a ligand for LFA-1, anti CD3, and a combination thereof. In some embodiments, the aAPC comprises on the cell surface at least two, at least 3, at least 4, or at least 5 exogenous costimulatory polypeptides.

In some embodiments, the aAPC further comprises on the cell surface an exogenous cytokine polypeptide. In some embodiments, the exogenous cytokine polypeptide is selected from the group consisting of: IL2, IL15, 15Rα fused to IL-15, IL7, IL12, IL18, IL21, IL4, IL6, IL23, IL27, IL17, IL10, TGF-beta, IFN-gamma, IL-1 beta, GM-CSF, and IL-25.

In some embodiments, the aAPC is capable of activating a T cell that interacts with the aAPC. In some embodiments, activating comprises activation of CD8+ T cells, activation of CD4+ T cells, stimulation of cytotoxic activity of T cells, stimulation of cytokine secretion by T cells, and/or any combination thereof.

In another aspect, the disclosure provides an artificial antigen presenting cell (aAPC) engineered to suppress T cell activity, wherein the aAPC comprises an enucleated cell, wherein the enucleated cell comprises on the cell surface an exogenous antigen-presenting polypeptide, an exogenous antigenic polypeptide and at least one exogenous co-inhibitory polypeptide disclosed in Table 7.

In another aspect, the disclosure provides an artificial antigen presenting cell (aAPC) engineered to suppress T cell activity, wherein the aAPC comprises an enucleated cell, wherein the enucleated cell comprises on the cell surface an exogenous antigen-presenting polypeptide, an exogenous antigenic polypeptide disclosed in Table 1 or Tables 16-19, and at least one exogenous co-inhibitory polypeptide.

In some embodiments, the aAPC further comprises a metabolite-altering polypeptide.

In another aspect, the disclosure provides an artificial antigen presenting cell (aAPC) engineered to suppress T cell activity, wherein the aAPC comprises an enucleated cell, wherein the enucleated cell comprises on the cell surface an exogenous antigen-presenting polypeptide, an exogenous antigenic polypeptide, and at least one metabolite-altering polypeptide.

In some embodiments, the aAPC further comprises an exogenous co-inhibitory polypeptide. In some embodiments, the exogenous co-inhibitory polypeptide is IL-35, IL-10, VSIG-3 or a LAG3 agonist. In some embodiments, the metabolite-altering polypeptide is IDO, Arg1, CD39, CD73, TDO, TPH, iNOS, COX2 or PGE synthase.

In some embodiments, the aAPC is capable of suppressing a T cell that interacts with the aAPC. In some embodiments, the suppressing comprises inhibition of proliferation of a T cell, anergizing of a T cell, or induction of apoptosis of a T cell. In some embodiments, the T cell is a CD4+ T cell or a CD8+ T cell.

In another aspect, the disclosure provides an artificial antigen presenting cell (aAPC) engineered to activate a regulatory T cell (Treg cell), wherein the aAPC comprises an enucleated cell, wherein the enucleated cell comprises on the cell surface an exogenous antigen-presenting polypeptide and an exogenous antigenic polypeptide.

In some embodiments, the aAPC further comprises on the cell surface an exogenous Treg expansion polypeptide. In some embodiments, the exogenous Treg expansion polypeptide is CD25-specific IL-2, TNFR2-specific TNF, antiDR3 agonist (VEGI/TL1A specific), 41BBL, TGFβ.

In some embodiments, the exogenous antigen-presenting polypeptide is an MHC class II polypeptide or an MHC class II single chain fusion protein. In some embodiments, the MHC class II polypeptide is selected from the group consisting of: HLA-DPα, HLA-DPβ, HLA-DM, HLA DOA, HLA DOB, HLA DQα, HLA DQβ, HLA DRα, and HLA DRβ. In some embodiments, the MHC class II single chain fusion protein comprises an α-chain and a β chain. In some embodiments, the MHC class II single chain fusion protein further comprises a membrane anchor. In some embodiments, the exogenous antigenic polypeptide is connected to the MHC class II single chain fusion via a linker. In some embodiments, the linker is a cleavable linker. In some embodiments, the anchor comprises a glycophorin A (GPA) protein or a transmembrane domain of small integral membrane protein 1 (SMIM1). In some embodiments, the exogenous antigenic polypeptide is bound to the exogenous antigen-presenting polypeptide covalently. In some embodiments, the exogenous antigenic polypeptide is bound to the exogenous antigen-presenting polypeptide non-covalently.

In some embodiments, the exogenous antigenic polypeptide is 8 amino acids in length to 24 amino acids in length.

In some embodiments, the enucleated cell is an enucleated erythroid cell or a platelet.

In another aspect, the disclosure provides a method of activating an antigen-specific T cell, the method comprising contacting the T cell with the aAPC of any one of the above aspects, thereby activating the antigen-specific T cell.

In another aspect, the disclosure provides a method for inducing proliferation of a T cell expressing a receptor molecule, the method comprising contacting the T cell with the aAPC of any one of the above aspects, wherein the costimulatory polypeptide specifically binds with the receptor molecule, thereby inducing proliferation of said T cell.

In another aspect, the disclosure provides a method of expanding a subset of a T cell population, the method comprising contacting a population of T cells comprising at least one T cell of the subset with an aAPC of any one of the above aspects, wherein the exogenous costimulatory polypeptide comprised on the surface of the aAPC specifically binds with a receptor molecule on the at least one T cell of the subset, and wherein binding of the exogenous costimulatory polypeptide to the receptor molecule induces proliferation of the at least one T cell of the subset, thereby expanding the subset of the T cell population.

In another aspect, the disclosure provides a method of suppressing activity of a T cell, the method comprising contacting the T cell with the aAPC of any one of the above aspects, thereby suppressing activity of the T cell.

In another aspect, the disclosure provides a method for activating a Treg cell, the method comprising contacting the Treg cell with the aAPC of any one of the above aspects, thereby activating the Treg cell.

In another aspect, the disclosure provides a method of treating a subject in need of an altered immune response, the method comprising contacting a T cell of the subject with the aAPC of any one of the above aspects, thereby treating the subject in need of an altered immune response.

In some embodiments, the contacting is in vitro. In some embodiments, the contacting is in vivo.

In another aspect, the disclosure provides a method of treating a subject in need of an altered immune response, the method comprising: a) determining an expression profile of an antigen on a cell in the subject, b) selecting an artificial antigen presenting cell (aAPC), wherein the aAPC is an engineered enucleated cell comprising on the cell surface an antigen-presenting polypeptide and at least one first exogenous antigenic polypeptide, and c) administering the aAPC to the subject, thereby treating the subject in need of the altered immune response.

In another aspect, the disclosure provides a method of treating a subject in need of an altered immune response, the method comprising: a) determining an HLA status of the subject, b) selecting an artificial antigen presenting cell (aAPC) that is immunologically compatible with the subject, wherein the aAPC is an engineered enucleated cell comprising on the cell surface at least one first exogenous antigenic polypeptide and at least one antigen-presenting polypeptide, and c) administering the aAPC to the subject, thereby treating the subject in need of the altered immune response.

In some embodiments, the subject is in need of an increased immune response. In some embodiments, the subject has cancer or an infectious disease. In some embodiments, the subject is in need of a decreased immune response. In some embodiments, the subject has an autoimmune disease or an allergic disease.

In another aspect, the disclosure provides a method of inducing a T cell response to an antigen in a subject in need thereof, said method comprising: obtaining a population of cells from the subject, wherein the population comprises a T cell, contacting the population of cells with the aAPC of any one of the above aspects, wherein contacting the population of cells with the aAPC induces proliferation of an antigen-specific T cell that is specific for the at least one exogenous antigenic polypeptide, and administering the antigen-specific T cell to the subject, thereby inducing a T cell response to the antigen in the subject in need thereof. In some embodiments, the method further comprises isolating the antigen-specific T cell from the population of cells.

In another aspect, the disclosure provides a method of expanding a population of regulatory T (Treg) cells, the method comprising: obtaining a population of cells from a subject, wherein the population comprises a Treg cell, contacting the population with the aAPC of any one of the above aspects, wherein contacting the population with the aAPC induces proliferation of the Treg cell, thereby expanding the population of Treg cells. In some embodiments, the method further comprises isolating the Treg cell from the population of cells. In some embodiments, the method further comprises administering the Treg cell to the subject.

In another aspect, the disclosure provides a method of making the aAPC of any one of the above aspects, the method comprising: introducing an exogenous nucleic acid encoding the exogenous antigenic polypeptide into a nucleated cell; and culturing the nucleated cell under conditions suitable for enucleation and for production of the exogenous antigenic polypeptide, thereby making an enucleated cell, thereby making the aAPC.

In one embodiment, the nucleated cell is a nucleated erythroid precursor cell. In one embodiment, the enucleated cell (e.g., engineered enucleated cell) is an enucleated erythroid cell, e.g., an erythrocyte or a reticulocyte. In one embodiment, the enucleated cell (e.g., engineered enucleated cell) is a platelet.

In another aspect, the disclosure provides a method of making the aAPC of any one of the above aspects, the method comprising: introducing an exogenous nucleic acid encoding the exogenous antigen-presenting polypeptide into a nucleated cell; culturing the nucleated cell under conditions suitable for enucleation and for production of the exogenous antigen-presenting polypeptide, thereby making an enucleated cell; and contacting the enucleated cell with at least one exogenous antigenic polypeptide, wherein the at least one exogenous antigenic polypeptide binds to the exogenous antigen-presenting polypeptide which is present on the cell surface of the enucleated cell, thereby making the aAPC.

In one embodiment, the at least one exogenous antigenic polypeptide specifically binds to the exogenous antigen-presenting polypeptide which is present on the cell surface of the enucleated cell

In one embodiment, the nucleated cell is a nucleated erythroid precursor cell. In one embodiment, the enucleated cell (e.g., engineered enucleated cell) is an enucleated erythroid cell, e.g., an erythrocyte or a reticulocyte. In one embodiment, the enucleated cell (e.g., engineered enucleated cell) is a platelet.

In another aspect, the disclosure provides a method of making the aAPC of any one of the above aspects, the method comprising: introducing an exogenous nucleic acid encoding the exogenous antigenic polypeptide into a nucleated cell; introducing an exogenous nucleic acid encoding the exogenous antigen-presenting polypeptide into the nucleated cell; and culturing the nucleated cell under conditions suitable for enucleation and for production of the exogenous antigenic polypeptide and the exogenous antigen-presenting polypeptide, thereby making an enucleated cell, thereby making the aAPC.

In one embodiment, the nucleated cell is a nucleated erythroid precursor cell. In one embodiment, the enucleated cell (e.g., engineered enucleated cell) is an enucleated erythroid cell, e.g., an erythrocyte or a reticulocyte. In one embodiment, the enucleated cell (e.g., engineered enucleated cell) is a platelet.

In some embodiments, the exogenous nucleic acid comprises DNA. In some embodiments, the exogenous nucleic acid comprises RNA.

In some embodiments, the introducing step comprises viral transduction. In some embodiments, the introducing step comprises electroporation. In some embodiments, the introducing step comprises utilizing one or more of: liposome mediated transfer, adenovirus, adeno-associated virus, herpes virus, a retroviral based vector, lipofection, and a lentiviral vector.

In another aspect, the disclosure provides a method of making an immunologically compatible artificial antigen presenting cell (aAPC), wherein the aAPC comprises an enucleated cell that comprises on the cell surface an exogenous antigenic polypeptide, the method comprising: contacting a nucleated cell with a nuclease and at least one gRNA which cleave an endogenous nucleic acid to result in production of an endogenous antigen-presenting polypeptide, an endogenous anchor polypeptide, or an endogenous costimulatory polypeptide; or to result in inhibition of expression of an endogenous microRNA; introducing an exogenous nucleic acid encoding the exogenous antigenic polypeptide into the nucleated cell; and culturing the nucleated cell under conditions suitable for enucleation and for production and presentation of the exogenous antigenic polypeptide by the endogenous antigen-presenting polypeptide, thereby making an enucleated cell, thereby making the immunologically compatible aAPC.

In some embodiments, the exogenous nucleic acid is contacted with a nuclease and at least one gRNA.

In yet another aspect, the disclosure features an artificial antigen presenting cell (aAPC) engineered to activate T cells, wherein the aAPC comprises an erythroid cell or enucleated cell, wherein the erythroid cell or enucleated cell presents, e.g., comprises on the cell surface, at least one exogenous antigenic polypeptide disclosed in Table 1.

In some embodiments, the at least one exogenous antigenic polypeptide is a tumor antigen, an autoimmune disease antigen, a viral antigen, a bacterial antigen or a parasite.

In another aspect, the disclosure features an artificial antigen presenting cell (aAPC) engineered to activate T cells, wherein the aAPC comprises an erythroid cell or enucleated cell, wherein the erythroid cell or enucleated cell presents, e.g., comprises on the cell surface, a first exogenous antigenic polypeptide and a second exogenous antigenic polypeptide, and wherein the first exogenous antigenic polypeptide and the second exogenous antigenic polypeptide have amino acid sequences which overlap by at least 2 amino acids.

In some embodiments, the overlap is between 2 amino acids and 23 amino acids.

In some embodiments, the aAPC further presents, e.g., comprises on the cell surface, an exogenous antigen-presenting polypeptide.

In some embodiments, the exogenous antigen-presenting polypeptide is an MHC class I polypeptide, an MHC class I single chain fusion, an MHC class II polypeptide, or an MHC class II single chain fusion.

In some embodiments, the MHC class I polypeptide is selected from the group consisting of: HLA A, HLA B, and HLA C.

In some embodiments, the MHC class II polypeptide is selected from the group consisting of: HLA-DPα, HLA-DPβ, HLA-DM, HLA DOA, HLA DOB, HLA DQα, HLA DQβ, HLA DRα, and HLA DRβ.

In some embodiments of the above aspects and embodiments, the erythroid cell is an enucleated erythroid cell.

In yet another aspect, the disclosure features an artificial antigen presenting cell (aAPC) engineered to activate T cells, wherein the aAPC comprises an erythroid cell or enucleated cell, wherein the erythroid cell or enucleated cell presents, e.g., comprises on the cell surface, an exogenous antigen-presenting polypeptide and an exogenous antigenic polypeptide, wherein the exogenous antigen-presenting polypeptide is an MHC class I single chain fusion or an MHC class II single chain fusion, wherein, e.g., the exogenous antigenic polypeptide is specifically bound to the exogenous antigen-presenting polypeptide.

In some embodiments, the MHC class I single chain fusion comprises an anchor, an α-chain, and a β2m chain. In some embodiments, the exogenous antigenic polypeptide is connected to the MHC I single chain fusion via a linker. In some embodiments, the linker is a cleavable linker.

In some embodiments, the MHC class II single chain fusion comprises an anchor, an α-chain, and a β chain. In some embodiments, the exogenous antigenic polypeptide is connected to the MHC II single chain fusion via a linker. In some embodiments, the linker is a cleavable linker. In some embodiments, the anchor is a Type 1 Membrane Protein. In some embodiments, the anchor is a Type 2 Membrane Protein. In some embodiments, the anchor is a GPI-linked protein. In some embodiments, the anchor is GPA or SMIM1.

In some embodiments, the exogenous antigenic polypeptide is bound to the exogenous antigen-presenting polypeptide. In some embodiments, the exogenous antigenic polypeptide is bound to the exogenous antigen-presenting polypeptide covalently or non-covalently.

In some embodiments, the aAPC of any one of the foregoing aspects further presents, e.g., comprises on the cell surface, at least one exogenous costimulatory polypeptide.

In some embodiments, the at least one exogenous costimulatory polypeptide is selected from the group consisting of 4-1BBL, LIGHT, anti CD28, CD80, CD86, CD70, OX40L, GITRL, TIM4, SLAM, CD48, CD58, CD83, CD155, CD112, IL-7, IL-12, IL-15Rα fused to IL-15, IL-21, ICAM-1, a ligand for LFA-1, anti CD3, and a combination thereof.

In some embodiments, the aAPC presents, e.g., comprises on the cell surface, at least two, at least 3, at least 4, or at least 5 exogenous costimulatory polypeptides.

In some embodiments, the aAPC is capable of activating a T cell that interacts with the aAPC. In some embodiments, the activating comprises activation of CD8+ T cells, activation of CD4+ T cells, stimulation of cytotoxic activity of T cells, stimulation of cytokine secretion by T cells, and/or any combination thereof.

In some embodiments of the above aspects and embodiments, the erythroid cell is an enucleated erythroid cell.

In still another aspect, the disclosure features an artificial antigen presenting cell (aAPC) engineered to suppress T cell activity, wherein the aAPC comprises an erythroid cell or enucleated cell, wherein the erythroid cell or enucleated cell presents, e.g., comprises on the cell surface, an exogenous antigen-presenting polypeptide, an exogenous antigenic polypeptide and at least one exogenous co-inhibitory polypeptide disclosed in Table 7, wherein, e.g., the exogenous antigenic polypeptide is specifically bound to the exogenous antigen-presenting polypeptide. In some embodiments, the erythroid cell is an enucleated erythroid cell.

In another aspect, the disclosure features an artificial antigen presenting cell (aAPC) engineered to suppress T cell activity, wherein the aAPC comprises an erythroid cell or enucleated cell, wherein the erythroid cell or enucleated cell presents, e.g., comprises on the cell surface, an exogenous antigen-presenting polypeptide, an exogenous antigenic polypeptide disclosed in Table 1, and at least one exogenous co-inhibitory polypeptide, wherein, e.g., the exogenous antigenic polypeptide is specifically bound to the exogenous antigen-presenting polypeptide. In some embodiments, the erythroid cell is an enucleated erythroid cell.

In some embodiments, the aAPC further comprises a metabolite-altering polypeptide.

In another aspect, the disclosure features an artificial antigen presenting cell (aAPC) engineered to suppress T cell activity, wherein the aAPC comprises an erythroid cell or enucleated cell, wherein the erythroid cell or enucleated cell presents, e.g., comprises on the cell surface, an exogenous antigen-presenting polypeptide, an exogenous antigenic polypeptide, and at least one metabolite-altering polypeptide, wherein, e.g., the exogenous antigenic polypeptide is specifically bound to the exogenous antigen-presenting polypeptide.

In some embodiments, the aAPC further comprising an exogenous co-inhibitory polypeptide. In some embodiments, the exogenous co-inhibitory polypeptide is IL-35, IL-10, VSIG-3, PD-L1 or a LAG3 agonist.

In some embodiments, the metabolite-altering polypeptide is IDO, Arg1, CD39, CD73, TDO, TPH, iNOS, COX2 or PGE synthase.

In some embodiments, the aAPC is capable of suppressing a T cell that interacts with the aAPC.

In some embodiments, the suppressing comprises inhibition of proliferation of a T cell, anergizing of a T cell, or induction of apoptosis of a T cell. In some embodiments, the T cell is a CD4+ T cell or a CD8+ T cell.

In some embodiments of the above aspects and embodiments, the erythroid cell is an enucleated erythroid cell.

In another aspect, the disclosure features an artificial antigen presenting cell (aAPC) engineered to activate a regulatory T cell (Treg cell), wherein the aAPC comprises an erythroid cell or enucleated cell, wherein the erythroid cell or enucleated cell presents, e.g., comprises on the cell surface, an exogenous antigen-presenting polypeptide and an exogenous antigenic polypeptide, wherein, e.g., the exogenous antigenic polypeptide is specifically bound to the exogenous antigen-presenting polypeptide.

In some embodiments, the aAPC further presents, e.g., comprises on the cell surface, an exogenous Treg expansion polypeptide.

In some embodiments, the exogenous antigen-presenting polypeptide is an MHC class II polypeptide or an MHC class II single chain fusion. In some embodiments, the MHC class II polypeptide is selected from the group consisting of: HLA-DPα, HLA-DPβ, HLA-DM, HLA DOA, HLA DOB, HLA DQα, HLA DQβ, HLA DRα, and HLA DRβ. In some embodiments, the MHC class II single chain fusion comprises an anchor, an α-chain, and a β chain.

In some embodiments, the exogenous antigenic polypeptide is connected to the MHC class II single chain fusion via a linker. In some embodiments, the linker is a cleavable linker.

In some embodiments, the anchor is GPA or SMIM1.

In some embodiments, the exogenous antigenic polypeptide is bound to the exogenous antigen-presenting polypeptide. In some embodiments, the exogenous antigenic polypeptide is bound to the exogenous antigen-presenting polypeptide covalently or non-covalently.

In some embodiments, the exogenous Treg expansion polypeptide is IL-2, CD25-specific IL-2, TNFR2-specific TNF, antiDR3 agonist (VEGI/TL1A specific), 4-1BBL, TGFβ.

In some embodiments, the exogenous antigenic polypeptide is 8 amino acids in length to 24 amino acids in length.

In some embodiments, the enucleated cell is an erythroid cell or a platelet.

In some embodiments of the above aspects and embodiments, the erythroid cell is an enucleated erythroid cell.

In another aspect, the disclosure features a method of activating an antigen-specific T cell, the method comprising contacting the T cell with an aAPC disclosed herein, thereby activating the antigen-specific T cell.

In another aspect, the disclosure features a method for inducing proliferation of a T cell expressing a receptor molecule, the method comprising contacting the T cell with an aAPC disclosed herein, wherein the costimulatory polypeptide specifically binds with the receptor molecule, thereby inducing proliferation of said T cell.

In another aspect, the disclosure features a method of expanding a subset of a T cell population, the method comprising contacting a population of T cells comprising at least one T cell of the subset with an aAPC disclosed herein, wherein the exogenous costimulatory polypeptide presented on the aAPC specifically binds with a receptor molecule on the at least one T cell of the subset, and wherein binding of exogenous costimulatory polypeptide to the receptor molecule induces proliferation of the at least one T cell of the subset, thereby expanding the subset of the T cell population.

In another aspect, the disclosure features a method of suppressing activity of a T cell, the method comprising contacting the T cell with an aAPC disclosed herein, thereby suppressing activity of the T cell.

In another aspect, the disclosure features a method for activating a Treg cell, the method comprising contacting the Treg cell with an aAPC disclosed herein, thereby activating the Treg cell.

In another aspect, the disclosure features a method of treating a subject in need of an altered immune response, the method comprising contacting a T cell of the subject with an aAPC as disclosed hereein, thereby treating the subject in need of an altered immune response.

In some embodiments, the contacting is in vitro or in vivo.

In another aspect, the disclosure features a method of treating a subject in need of an altered immune response, the method comprising: a) determining an expression profile of an antigen on a cell in the subject; b) selecting an artificial antigen presenting cell (aAPC), wherein the aAPC is an engineered erythroid cell expressing an antigen-presenting polypeptide and at least one first exogenous antigenic polypeptide; and c) administering the aAPC to the subject, thereby treating the subject in need of the altered immune response.

In another aspect, the disclosure features a method of treating a subject in need of an altered immune response, the method comprising: a) determining an HLA status of the subject; b) selecting an artificial antigen presenting cell (aAPC) that is immunologically compatible with the subject, wherein the aAPC is an engineered erythroid cell expressing at least one first exogenous antigenic polypeptide and at least one antigen-presenting polypeptide; and c) administering the aAPC to the subject, thereby treating the subject in need of the altered immune response.

In some embodiments, the subject is in need of an increased immune response. In some embodiments, the subject has cancer or an infectious disease. In some embodiments, the subject is in need of a decreased immune response. In some embodiments, the subject has an autoimmune disease or an allergic disease.

In another aspect, the disclosure features a method of inducing a T cell response to an antigen in a subject in need thereof, said method comprising: obtaining a population of cells from the subject, wherein the population comprises a T cell; contacting the population of cells with an aAPC disclosed herein, wherein contacting the population of cells with the aAPC induces proliferation of an antigen-specific T cell that is specific for the at least one exogenous antigenic polypeptide, and administering the antigen-specific T cell to the subject, thereby inducing a T cell response to the antigen in the subject in need thereof.

In some embodiments, the method further comprises isolating the antigen-specific T cell from the population of cells.

In another aspect, the disclosure features a method of expanding a population of regulatory T (Treg) cells, the method comprising: obtaining a population of cells from the subject, wherein the population comprises a Treg cell; contacting the population with an aAPC disclosed herein, wherein contacting the population with the aAPC induces proliferation of the Treg cell, thereby expanding the population of Treg cells.

In some embodiments, the method further comprises isolating the Treg cell from the population of cells.

In some embodiments, the method further comprises administering the Treg cell to the subject.

In some embodiments of each of the above methods, the erythroid cell is an enucleated erythroid cell.

In another aspect, the disclosure features a method of making an aAPC of the invention, the method comprising: introducing an exogenous nucleic acid encoding the exogenous antigenic polypeptide into a nucleated cell; and culturing the nucleated cell under conditions suitable for expression and presentation of the exogenous antigenic polypeptide, and enucleation, thereby making an enucleated cell, thereby making the aAPC.

In another aspect, the disclosure features a method of making an aAPC of the invention, the method comprising: introducing an exogenous nucleic acid encoding the exogenous antigen-presenting polypeptide into a nucleated cell; culturing the nucleated cell under conditions suitable for expression and presentation of the exogenous antigen-presenting polypeptide, and enucleation, thereby making an enucleated cell; and contacting the enucleated cell with at least one exogenous antigenic polypeptide, wherein the at least one exogenous antigenic polypeptide binds to the exogenous antigen-presenting polypeptide which is presented on the enucleated cell, thereby making the aAPC.

In some embodiments, the exogenous nucleic acid comprises DNA or RNA.

In some embodiments, the introducing step comprises viral transduction. In some embodiments, the introducing step comprises electroporation. In some embodiments, the introducing step comprises utilizing one or more of: liposome mediated transfer, adenovirus, adeno-associated virus, herpes virus, a retroviral based vector, lipofection, and a lentiviral vector.

In another aspect, the disclosure features a method of making an immunologically compatible artificial antigen presenting cell (aAPC), wherein the aAPC comprises an enucleated cell that presents, e.g. comprises on the cell surface, an exogenous antigenic polypeptide, the method comprising: contacting a nucleated cell with a nuclease and at least one gRNA which cleave an endogenous nucleic acid to result in expression of an endogenous antigen-presenting polypeptide, an endogenous anchor polypeptide, or an endogenous costimulatory polypeptide; or to result in inhibition of expression of an endogenous microRNA; introducing an exogenous nucleic acid encoding the exogenous antigenic polypeptide into the nucleated cell; and culturing the nucleated cell under conditions suitable for expression and presentation of the exogenous antigenic polypeptide by the endogenous antigen-presenting polypeptide, and enucleation, thereby making an enucleated cell, thereby making the immunologically compatible aAPC.

In some embodiments, the exogenous nucleic acid is contacted with a nuclease and at least one gRNA.

In some embodiments of any of the above aspects and embodiments, the erythroid cell is an enucleated erythroid cell.

### BRIEF DESCRIPTION OF THE DRAWINGS

The figures are meant to be illustrative of one or more features, aspects, or embodiments of the invention and are not intended to be limiting.

FIGS. 1A & FIG. 1B are schematics showing various designs for expressing MHCI and MHCII molecules on erythroid cells. FIG. 1A shows a schematic of the design for expressing single-chain peptide-MHCII constructs. As shown in FIG. 1A, an exogenous peptide is linked to the MHCII β-chain, linked to the MHCII α-chain, linked to a membrane anchor, such as GPA or SMIM. FIG. 1B shows a schematic of the design for expressing single-chain peptide-MHCI constructs. As shown in FIG. 1B, an exogenous peptide is linked to the MHCI β-2m subunit, linked to the MHCI α subunit linked to a membrane anchor, such as GPA or SMIM.

FIG. 2 is a graph showing that engineered murine erythrocytes presenting MHC I (ovalbumin) and 4-1BBL activate ova-specific T cells.

FIG. 3 is a graph showing that ova-specific T cells expanded with murine erythrocytes presenting MHC I (ovalbumin) and 4-1BBL are highly potent and specific in tumor cell killing.

FIG. 4A is a schematic showing the experimental design to study the proliferation of OT1-T cells in lymph nodes and spleen

FIG. 4B is a schematic of representative data, showing that mRCT-4-1BBL OVA specifically expand and activate OT1-T cells, while mRCT-4-1BBL without MHCI presenting ovalbumin peptide on the cell surface do not expand and activate OT1-T cells. As used herein throughout, mouse red cell therapeutic (or mRCT) refers to murine engineered erythroid cells (e.g. an engineered enucleated cell) described herein. As used herein throughout, RCT (red cell therapeutic) refers to human engineered erythroid cells (e.g. an engineered enucleated cell) described herein.

FIG. 4C is a graph showing in vivo observations for the proliferation and activation of OT1-T cells by mRCT-4-1BBL OVA in circulation, spleen and lymph node.

FIG. 5A-D are graphs showing erythroid cells engineered to present MHCI (ovalbumin) and 4-1BBL exhibit an in vivo dose response ova-specific T cells in vivo.

FIG. 6 is a graph showing that a second dose of the erythroid cells engineered to present MHCI (ovalbumin) and 4-1BBL dramatically boosts CD8+OT1 T-Cells in both lymph node and spleen.

FIG. 7 is a graph showing that erythroid cells engineered to present MHCI (gp100) and 4-1BBL activate gp100-specific T cells in vitro.

FIG. 8A is a schematic showing the different versions of HLA-A2 (HPV E7) expressed on RCTs. FIG. 8A discloses “YMLDLQPETGGGGS(G4S)2” as SEQ ID NO: 895 and “(G4S)4” as SEQ ID NO: 733.

FIGS. 8B and 8C are graphs showing the activity of HLA-A2 (HPV E7) expressed on RCTs, in stimulating HPV-specific T cells in vitro.

FIG. 9 is a graph showing the change in average tumor volume (mm³) over time after tumor randomization, where mice are dosed with mRCT (control) and mRCT-OVA-4-1BBL at days 1, 4 and 8 after OT1 CD8+ T cell injection.

FIG. 10 is a graph showing the change in individual tumor volume (mm³) over time after tumor randomization, where mice are dosed with mRCT (control) and mRCT-OVA-4-1BBL at days 1, 4 and 8 after OT1 CD8+ T cell injection.

FIG. 11 is a graph showing percent survival of mice over time after tumor randomization, where mice are dosed with mRCT (control) and mRCT-OVA-4-1BBL at days 1, 4 and 8 after OT1 CD8+ T cell injection.

FIG. 12 shows the results of flow cytometry experiments, gating for CD44+ expression, to determine OT1 CD8+ T cell proliferation.

FIG. 13 is a graph showing OT1 CD8+ T cell count at day 4 following coincubation of mRCTs (control and clicked) with OT1 CD8+ T cells. FIG. 14A is a graph showing that triple clicked mRCTs (mRCT-OVA-4-1BBL-IL7, mRCT-OVA-4-1BBL-IL12, or mRCT-OVA-4-1BBL-IL15), show increased OT1 CD8+ T cell proliferation over the double clicked mRCTs (mRCT-OVA-4-1BBL).

FIG. 14B is a panel of graphs showing percentages of memory stem T cells (Tscm), central memory T cells (Tcm) and effector memory T cells (Tem) activated by the double clicked mRCTs (mRCT-OVA-4-1BBL), or triple clicked mRCTs (mRCT-OVA-4-1BBL-IL7, mRCT-OVA-4-1BBL-IL12, or mRCT-OVA-4-1BBL-IL15).

FIGS. 15A and 15B are graphs showing that the mice treated with mRCT-OVA-4-1BBL demonstrate EG7.OVA tumor control even upon being re-challenged with EG7.OVA tumor cells.

FIG. 16A is a schematic showing the timeline of mice being re-challenged with OVA peptide (SIINFEKL (SEQ ID NO: 721))+ Incomplete Freund's adjuvant (IFA).

FIG. 16B is a graph showing that mice treated with mRCT-OVA had lower OT1 cell counts upon OVA peptide re-challenge as compared to mice dosed only with mRCT in both spleen and lymph node.

FIG. 16C is a graph showing that the endogenous CD8+ T cell counts were not impacted upon OVA peptide re-challenge as compared to mice dosed only with mRCT in both spleen and lymph node.

FIG. 17A is a schematic showing the different options of configurations, for presenting signals 1 and 2 on the surface of an RCT.

FIG. 17B is a schematic showing the different options of configurations, for presenting signals 1, 2 and 3 on the surface of an RCT.

### DETAILED DESCRIPTION

The present disclosure is based on the development of artificial antigen presenting cells (aAPCs) with efficient signal presentation, that can be used for, e.g. in vivo aAPC immunotherapy and ex vivo for T cell expansion. In particular, the present disclosure is based, at least in part, upon the surprising finding that erythroid cells, and in particular engineered erythroid cells, can be engineered to, inter alia, activate, expand or differentiate/de-differentiate T cells, suppress T cell activity, suppress T effector cells, and/or stimulate and expand T regulatory cells.

Many modifications and other embodiments of the inventions set forth herein will easily come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### Definitions

As used in this specification and the appended claims, the singular forms “a”, “an” and “the” include plural references unless the content clearly dictates otherwise.

The use of the alternative (e.g., “or”) should be understood to mean either one, both, or any combination thereof of the alternatives.

As used herein, the term “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, preferred materials and methods are described herein.

As used herein, “administration,” “administering” and variants thereof refers to introducing a composition or agent into a subject and includes concurrent and sequential introduction of a composition or agent. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, the term an “antigen-presenting cell (APC)” refers to a cell that can process and display foreign antigens in association with major histocompatibility complex (MHC) molecules on its surface.

As used herein, the term an “artificial antigen presenting cell” refers to cells that have been engineered to introduce one or more molecules (e.g. exogenous polypeptides) that provide the necessary T cell receptor (TCR), costimulatory, and/or adhesion events required for immune synapse formation.

As used herein, the term “autoimmune disorders” refers generally to conditions in which a subject's immune system attacks the body's own cells, causing tissue destruction. Autoimmune disorders may be diagnosed using blood tests, cerebrospinal fluid analysis, electromyogram (measures muscle function), and magnetic resonance imaging of the brain, but antibody testing in the blood, for self-antibodies (or auto-antibodies) is particularly useful. Usually, IgG class antibodies are associated with autoimmune diseases.

As used herein, the term “biological sample” refers to any type of material of biological origin isolated from a subject, including, for example, DNA, RNA, lipids, carbohydrates, and protein. The term “biological sample” includes tissues, cells and biological fluids isolated from a subject. Biological samples include, e.g., but are not limited to, whole blood, plasma, serum, semen, saliva, tears, urine, fecal material, sweat, buccal, skin, cerebrospinal fluid, bone marrow, bile, hair, muscle biopsy, organ tissue or other material of biological origin known by those of ordinary skill in the art. Biological samples can be obtained from subjects for diagnosis or research or can be obtained from healthy subjects, as controls or for basic research.

As used herein, the term “cancer” refers to diseases in which abnormal cells divide without control and are able to invade other tissues. There are more than 100 different types of cancer. Most cancers are named for the organ or type of cell in which they start—for example, cancer that begins in the colon is called colon cancer; cancer that begins in melanocytes of the skin is called melanoma. Cancer types can be grouped into broader categories. The main categories of cancer include: carcinoma (meaning a cancer that begins in the skin or in tissues that line or cover internal organs, and its subtypes, including adenocarcinoma, basal cell carcinoma, squamous cell carcinoma, and transitional cell carcinoma); sarcoma (meaning a cancer that begins in bone, cartilage, fat, muscle, blood vessels, or other connective or supportive tissue); leukemia (meaning a cancer that starts in blood-forming tissue (e.g., bone marrow) and causes large numbers of abnormal blood cells to be produced and enter the blood; lymphoma and myeloma (meaning cancers that begin in the cells of the immune system); and central nervous system (CNS) cancers (meaning cancers that begin in the tissues of the brain and spinal cord). The term “myelodysplastic syndrome” refers to a type of cancer in which the bone marrow does not make enough healthy blood cells (white blood cells, red blood cells, and platelets) and there are abnormal cells in the blood and/or bone marrow. Myelodysplastic syndrome may become acute myeloid leukemia (AML). In certain embodiments, the cancer is selected from cancers including, but not limited to, ACUTE lymphoblastic leukemia (ALL), ACUTE myeloid leukemia (AML), anal cancer, bile duct cancer, bladder cancer, bone cancer, bowel cancer, brain tumour, breast cancer, cancer of unknown primary, cancer spread to bone, cancer spread to brain, cancer spread to liver, cancer spread to lung, carcinoid, cervical cancer, choriocarcinoma, chronic lymphocytic leukemia (CLL), chronic myeloid leukemia (CML), colon cancer, colorectal cancer, endometrial cancer, eye cancer, gallbladder cancer, gastric cancer, gestational trophoblastic tumour (GTT), hairy cell leukemia, head and neck cancer, Hodgkin lymphoma, kidney cancer, laryngeal cancer, leukemia, liver cancer, lung cancer, lymphoma, melanoma skin cancer, mesothelioma, men's cancer, molar pregnancy, mouth and oropharyngeal cancer, myeloma, nasal and sinus cancers, nasopharyngeal cancer, non hodgkin lymphoma (NHL), oesophageal cancer, ovarian cancer, pancreatic cancer, penile cancer, prostate cancer, rare cancers, rectal cancer, salivary gland cancer, secondary cancers, skin cancer (non melanoma), soft tissue sarcoma, stomach cancer, testicular cancer, thyroid cancer, unknown primary cancer, uterine cancer, vaginal cancer, and vulval cancer.

As used herein, the term “click reaction” refers to a range of reactions used to covalently link a first and a second moiety, for convenient production of linked products. It typically has one or more of the following characteristics: it is fast, is specific, is high-yield, is efficient, is spontaneous, does not significantly alter biocompatibility of the linked entities, has a high reaction rate, produces a stable product, favors production of a single reaction product, has high atom economy, is chemoselective, is modular, is stereoselective, is insensitive to oxygen, is insensitive to water, is high purity, generates only inoffensive or relatively non-toxic by-products that can be removed by nonchromatographic methods (e.g., crystallization or distillation), needs no solvent or can be performed in a solvent that is benign or physiologically compatible, e.g., water, stable under physiological conditions. Examples include an alkyne/azide reaction, a diene/dienophile reaction, or a thiol/alkene reaction. Other reactions can be used. In some embodiments, the click reaction is fast, specific, and high-yield.

As used herein, the term “click handle” refers to a chemical moiety that is capable of reacting with a second click handle in a click reaction to produce a click signature. In some embodiments, a click handle is comprised by a coupling reagent, and the coupling reagent may further comprise a substrate reactive moiety.

As used herein, the term “cytokine” refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Cytokines can act both locally and distantly from a site of release. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of other cytokines. Non limiting examples of cytokines include e.g., IL-1α, IL-β, IL-2, IL-3, IL-4, IL-5, IL-6, IL-7, IL-8, IL-9, IL-10, IL-11, IL-12/IL-23 P40, IL13, IL-15, IL-17, IL-18, IL-21, IL-23, TGF-β, IFN-γ, GM-CSF, Groα, MCP-1 and TNF-α.

As used herein, the term “endogenous” is meant to refer to a native form of compound (e.g., a small molecule) or process. For example, in some embodiments, the term “endogenous” refers to the native form of a nucleic acid or polypeptide in its natural location in the organism or in the genome of an organism.

As used herein, the term “an engineered cell” as used herein is a genetically-modified cell or progeny thereof. In some embodiments, an engineered cell (e.g. an engineered enucleated cell) can be produced using coupling reagents to link an exogenous polypeptide to the surface of the cell (e.g. using click chemistry).

As used herein, the term “enucleated” refers to a cell, e.g., a reticulocyte or mature red blood cell (erythrocyte), that lacks a nucleus. In an embodiment an enucleated cell is a cell that has lost its nucleus through differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell, into a reticulocyte or mature red blood cell. In an embodiment an enucleated cell is a cell that has lost its nucleus through in vitro differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell into a reticulocyte or mature red blood cell. In an embodiment an enucleated cell lacks DNA. In an embodiment an enucleated cell is incapable of expressing a polypeptide, e.g., incapable of transcribing and/or translating DNA into protein, e.g., lacks the cellular machinery necessary to transcribe and/or translate DNA into protein. In some embodiments, an enucleated cell is an erythrocyte, a reticulocyte, or a platelet.

In some embodiments, the enucleated cells are not platelets, and therefore are “platelet free enucleated” cells (“PFE” cells). It should be understood that platelets do not have nuclei, and in this particular embodiment, platelets are not intended to be encompassed.

As used herein, “erythroid cell” includes a nucleated red blood cell, a red blood cell precursor, an enucleated mature red blood cell, and a reticulocyte. As used herein, an erythroid cell includes an erythroid precursor cell, a cell capable of differentiating into a reticulocyte or erythrocyte. For example, erythroid precursor cells include any of a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast. A preparation of erythroid cells can include any of these cells or a combination thereof. In some embodiments, the erythroid precursor cells are immortal or immortalized cells. For example, immortalized erythroblast cells can be generated by retroviral transduction of CD34+ hematopoietic progenitor cells to express Oct4, Sox2, Klf4, cMyc, and suppress TP53 (e.g., as described in Huang et al., Mol Ther (2014) Mol. Ther. 22(2): 451-63, the entire contents of which are incorporated by reference herein). In addition, the cells may be intended for autologous use or provide a source for allogeneic transfusion. In some embodiments, erythroid cells are cultured. In an embodiment an erythroid cell is an enucleated red blood cell.

As used herein, the term “exogenous,” when used in the context of nucleic acid, includes a transgene and recombinant nucleic acids.

As used herein, the term “exogenous nucleic acid” refers to a nucleic acid (e.g., a gene) which is not native to a cell, but which is introduced into the cell or a progenitor of the cell. An exogenous nucleic acid may include a region or open reading frame (e.g., a gene) that is homologous to, or identical to, an endogenous nucleic acid native to the cell. In some embodiments, the exogenous nucleic acid comprises RNA. In some embodiments, the exogenous nucleic acid comprises DNA. In some embodiments, the exogenous nucleic acid is integrated into the genome of the cell. In some embodiments, the exogenous nucleic acid is processed by the cellular machinery to produce an exogenous polypeptide. In some embodiments, the exogenous nucleic acid is not retained by the cell or by a cell that is the progeny of the cell into which the exogenous nucleic acid was introduced.

As used herein, the term “exogenous polypeptide” refers to a polypeptide that is not produced by a wild-type cell of that type or is present at a lower level in a wild-type cell than in a cell containing the exogenous polypeptide. In some embodiments, an exogenous polypeptide refers to a polypeptide that is introduced into or onto a cell, or is caused to be expressed by the cell by introducing an exogenous nucleic acid encoding the exogenous polypeptide into the cell or into a progenitor of the cell. In some embodiments, an exogenous polypeptide is a polypeptide encoded by an exogenous nucleic acid that was introduced into the cell or a progenitor of the cell, which nucleic acid is optionally not retained by the cell. In some embodiments, an exogenous polypeptide is a polypeptide conjugated to the surface of the cell by chemical or enzymatic means.

As used herein, the term “express” or “expression” refers to the process to produce a polypeptide, including transcription and translation. Expression may be, e.g., increased by a number of approaches, including: increasing the number of genes encoding the polypeptide, increasing the transcription of the gene (such as by placing the gene under the control of a constitutive promoter), increasing the translation of the gene, knocking out of a competitive gene, or a combination of these and/or other approaches.

As used herein, the terms “first”, “second”, and “third”, etc., with respect to exogenous polypeptides or nucleic acids are used for convenience of distinguishing when there is more than one type of exogenous polypeptide or nucleic acid. Use of these terms is not intended to confer a specific order or orientation of the exogenous polypeptides or nucleic acid unless explicitly so stated.

The term “flow cytometry” as used herein refers to a tool for interrogating the phenotype and characteristics of cells. It senses cells or particles as they move in a liquid stream through a laser (light amplification by stimulated emission of radiation)/light beam past a sensing area. The relative light-scattering and color-discriminated fluorescence of the microscopic particles is measured. Flow Analysis and differentiation of the cells is based on size, granularity, and whether the cells are carrying fluorescent molecules in the form of either antibodies or dyes. As the cell passes through the laser beam, light is scattered in all directions, and the light scattered in the forward direction at low angles)(0.5-10° from the axis is proportional to the square of the radius of a sphere and so to the size of the cell or particle. Light may enter the cell; thus, the 90° light (right-angled, side) scatter may be labeled with fluorochrome-linked antibodies or stained with fluorescent membrane, cytoplasmic, or nuclear dyes. Thus, the differentiation of cell types, the presence of membrane receptors and antigens, membrane potential, pH, enzyme activity, and DNA content may be facilitated. Flow cytometers are multiparameter, recording several measurements on each cell; therefore, it is possible to identify a homogeneous subpopulation within a heterogeneous population (Marion G. Macey, Flow cytometry: principles and applications, Humana Press, 2007). Fluorescence-activated cell sorting (FACS), which allows isolation of distinct cell populations too similar in physical characteristics to be separated by size or density, uses fluorescent tags to detect surface proteins that are differentially expressed, allowing fine distinctions to be made among physically homogeneous populations of cells.

As used herein, the term “gene” is used broadly to refer to any segment of nucleic acid associated with expression of a given RNA or protein. Thus, genes include regions encoding expressed RNAs (which typically include polypeptide coding sequences) and, often, the regulatory sequences required for their expression. Genes can be obtained from a variety of sources, including cloning from a source of interest or synthesizing from known or predicted sequence information, and may include sequences designed to have specifically desired parameters.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” refers to a primary response induced by ligation of a cell surface moiety. For example, in the context of receptors, such stimulation entails the ligation of a receptor and a subsequent signal transduction event. With respect to stimulation of a T cell, such stimulation refers to the ligation of a T cell surface moiety that in some embodiments subsequently induces a signal transduction event, such as binding the TCR/CD3 complex. Further, the stimulation event may activate a cell and upregulate or downregulate expression or secretion of a molecule. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses. “Activation” includes activation of CD8+ T cells, activation of CD4+ T cells, stimulation of cytotoxic activity of T cells, stimulation of cytokine secretion by T cells, detectable effector functions, modification of the differentiation state of a T cell (e.g. promote expansion and differentiation from T effector to T memory cell), and/or any combination thereof. The term “activated T cells” refers to, among other things, T cells that are undergoing cell division.

As used herein, “altered immune response” refers to changing the form or character of the immune response, for example stimulation or inhibition of the immune response, e.g., as measured by ELISPOT assay (cellular immune response), ICS (intracellular cytokine staining assay) and major histocompatibility complex (MHC) tetramer assay to detect and quantify antigen-specific T cells, quantifying the blood population of antigen-specific CD4+ T cells, or quantifying the blood population of antigen specific CD8+ T cells by a measurable amount, or where the increase is by at least 10%, at least 20%, at least 30%, at least 40%, at least 50%, at least 60%, at least 70%, at least 80%, at least 90%, at least 95%, at least 96%, at least 97%, at least 98%, at least 99%, at least 100%, when compared to a suitable control (e.g., a control composition where dendritic cells are not loaded with tumor-specific cells, or not loaded with peptide derived from tumor-specific cells).

As used herein, polypeptides referred to herein as “recombinant” refer to polypeptides which have been produced by recombinant DNA methodology, including those that are generated by procedures which rely upon a method of artificial recombination, such as the polymerase chain reaction (PCR) and/or cloning into a vector using restriction enzymes.

As used herein, a “single-chain antibody (scFv)” refers to an antibody in which a V_{L} and a V_{H} region are joined via a linker (e.g., a synthetic sequence of amino acid residues) to form a continuous protein chain. The linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83).

The term “specifically binds,” as used herein refers to the ability of a polypeptide or polypeptide complex to recognize and bind to a ligand in vitro or in vivo while not substantially recognizing or binding to other molecules in the surrounding milieu. In some embodiments, specific binding can be characterized by an equilibrium dissociation constant of at least about 1×10⁶M or less (e.g., a smaller equilibrium dissociation constant denotes tighter binding). Methods for determining whether two molecules specifically bind are well known in the art and include, for example, equilibrium dialysis, surface plasmon resonance, and the like.

As used herein, the terms “subject,” “individual,” “host,” and “patient,” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in particular embodiments the subject is a human.

As used herein, the phrase “subject in need” refers to a subject that (i) will be administered an aAPC (or pharmaceutical composition comprising an aAPC) according to the described invention, (ii) is receiving an aAPC (or pharmaceutical composition comprising an aAPC) according to the described invention; or (iii) has received an aAPC (or pharmaceutical composition comprising an aAPC) according to the described invention, unless the context and usage of the phrase indicates otherwise

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the terms “suppressing immune cells” or “inhibiting immune cells” refer to a process (e.g., a signaling event) causing or resulting in the inhibition or suppression of one or more cellular responses or activities of an immune cell, selected from: proliferation, differentiation, cytokine secretion, cytotoxic effector molecule release, cytotoxic activity, and expression of activation markers, or resulting in anergizing of an immune cell or induction of apoptosis of an immune cell. Suitable assays to measure immune cell inhibition or suppression are known in the art and are described herein.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the US Federal government or listed in the US Pharmacopeia for use in animals, including humans, as well as any carrier or diluent that does not cause significant irritation to a subject and does not abrogate the biological activity and properties of the administered compound.

As used herein, the terms “therapeutic amount”, “therapeutically effective amount”, an “amount effective”, or “pharmaceutically effective amount” of an active agent (e.g. an aAPC as described herein) are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

The term “exogenous antigenic polypeptide” as used herein refers to an exogenous polypeptide that is capable of inducing an immune response. An exogenous antigenic polypeptide is capable of binding to exogenous antigen-presenting polypeptide.

The term “exogenous antigen-presenting polypeptide” as used herein refers to a set of cell surface proteins that bind antigens and display them on the cell surface for recognition by the appropriate T-cells. The MHC gene family is divided into three subgroups: class I, class II, and class III. MHC class I molecules are heterodimers that consist of two polypeptide chains, an α chain and a β2-microglobulin (b2m) chain. Class I MHC molecules have β2 subunits so can only be recognized by CD8 co-receptors. MHC class II molecules are also heterodimers that consist of an α and β polypeptide chain. The subdesignation of chains as e.g., α1, α2, etc. refers to separate domains within the HLA gene. Class II MHC molecules have β1 and β2 subunits and can be recognized by CD4 co-receptors. The human MHC is also called the HLA (human leukocyte antigen) complex. In some embodiments, an “exogenous antigen-presenting polypeptide” refers to the cell surface proteins HLA-A, HLA-B, HLA-C, HLA-DRB1, HLA-DQA1, HLA-DQB1, HLA-DPA1, HLA-DPB1, that are capable of binding antigens and displaying them on the cell surface. Exogenous antigen-presenting polypeptides are described in more detail below.

The term “exogenous T cell costimulatory polypeptide” as used herein, includes a polypeptide on an antigen presenting cell (e.g., an aAPC) that specifically binds a cognate co-stimulatory molecule on a T cell (e.g., an MHC molecule, B and T lymphocyte attenuator (CD272), and a Toll like receptor), thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory polypeptide also encompasses, inter alia, an antibody that specifically binds with a co-stimulatory molecule present on a T cell. Exemplary exogenous co-stimulatory polypeptides are described in more detail below.

The term “exogenous T cell co-inhibitory polypeptide” as used herein refers to any polypeptide that suppresses a T cell, including inhibition of T cell activity, inhibition of T cell proliferation, anergizing of a T cell, or induction of apoptosis of a T cell. Exemplary exogenous co-inhibitory polypeptides are described in more detail below.

The term “exogenous metabolite-altering polypeptide” as used herein refers to any polypeptide involved in the catabolism or anabolism of a metabolite in a cell, wherein the metabolite-altering polypeptide can affect the metabolism of a T cell. Exemplary metabolite-altering polypeptides are described in more detail below.

The term “Treg costimulatory polypeptide” as used herein refers to an exogenous polypeptide that expands regulatory T-cells (Tregs). In some embodiments, a Treg costimulatory polypeptide stimulates Treg cells by stimulating at least one of three signals involved in Treg cell development. Exemplary exogenous Treg co-stimulatory polypeptides are described in more detail below.

As used herein, the terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well. In some embodiments, the peptide is of any length or size.

As used herein the term “nucleic acid molecule” refers to a single or double-stranded polymer of deoxyribonucleotide or ribonucleotide bases. It includes chromosomal DNA and self-replicating plasmids, vectors, mRNA, tRNA, siRNA, etc. which may be recombinant and from which exogenous polypeptides may be expressed when the nucleic acid is introduced into a cell.

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity.” (a) The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence. (b) The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches. Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences, 8:155-65 (1992), and Pearson, et al., Methods in Molecular Biology, 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995). Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915). In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar A number of low-complexity filter programs may be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters may be employed alone or in combination. (c) The term “sequence identity” or “identity” in the context of two nucleic acid or polypeptide sequences is used herein to refer to the residues in the two sequences that are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA). (d) The term “percentage of sequence identity” is used herein mean the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. (e) The term “substantial identity” of polynucleotide sequences means that a polynucleotide comprises a sequence that has at least 70% sequence identity, at least 80% sequence identity, at least 90% sequence identity and at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid. Mutations may also be made to the nucleotide sequences of the present proteins by reference to the genetic code, including taking into account codon degeneracy.

As used herein, the term “variant” refers to a polypeptide which differs from the original protein by one or more amino acid substitutions, deletions, insertions, or other modifications. These modifications do not significantly change the biological activity of the original protein. In many cases, a variant retains at least 10%, 20%, 30%, 40%, 50%, 60%, 70%, 80%, 90%, 95%, or 100% of the biological activity of original protein. The biological activity of a variant can also be higher than that of the original protein. A variant can be naturally-occurring, such as by allelic variation or polymorphism, or be deliberately engineered.

The amino acid sequence of a variant is substantially identical to that of the original protein. In many embodiments, a variant shares at least 50%, 60%, 70%, 75%, 80%, 85%, 90%, 91%, 92%, 93%, 94%, 95%, 96%, 97%, 98%, or 99%, or more global sequence identity or similarity with the original protein. Sequence identity or similarity can be determined using various methods known in the art, such as Basic Local Alignment Tool (BLAST), dot matrix analysis, or the dynamic programming method. In one example, the sequence identity or similarity is determined by using the Genetics Computer Group (GCG) programs GAP (Needleman-Wunsch algorithm) The amino acid sequences of a variant and the original protein can be substantially identical in one or more regions, but divergent in other regions. A variant may include a fragment (e.g., a biologically active fragment of a polypeptide). In some embodiments, a fragment may lack up to about 1, 2, 3, 4, 5, 10, 20, 30, 40, 50, or 100 amino acid residues on the N-terminus, C-terminus, or both ends (each independently) of a polypeptide, as compared to the full-length polypeptide.

### I. Cells of the Immune System

There are a large number of cellular interactions that comprise the immune system. These interactions occur through specific receptor-ligand pairs that signal in both directions so that each cell receives instructions based on the temporal and spatial distribution of those signals.

Murine models have been highly useful in discovering immunomodulatory pathways, but clinical utility of these pathways does not always translate from an inbred mouse strain to an outbred human population, since an outbred human population may have individuals that rely to varying extents on individual immunomodulatory pathways.

Cells of the immune system include lymphocytes, monocytes/macrophages, dendritic cells, the closely related Langerhans cells, natural killer (NK) cells, mast cells, basophils, and other members of the myeloid lineage of cells. In addition, a series of specialized epithelial and stromal cells provide the anatomic environment in which immunity occurs, often by secreting critical factors that regulate growth and/or gene activation in cells of the immune system, which also play direct roles in the induction and effector phases of the response. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

The cells of the immune system are found in peripheral organized tissues, such as the spleen, lymph nodes, Peyer's patches of the intestine and tonsils. Lymphocytes also are found in the central lymphoid organs, the thymus, and bone marrow where they undergo developmental steps that equip them to mediate the myriad responses of the mature immune system. A substantial portion of lymphocytes and macrophages comprise a recirculating pool of cells found in the blood and lymph, providing the means to deliver immunocompetent cells to sites where they are needed and to allow immunity that is generated locally to become generalized. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens through recombination of their genetic material (e.g. to create a T cell receptor and a B cell receptor). This commitment, which exists before the first contact of the immune system with a given antigen, is expressed by the presence of receptors specific for determinants (epitopes) on the antigen on the lymphocyte's surface membrane. Each lymphocyte possesses a unique population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

Two broad classes of lymphocytes are recognized: the B-lymphocytes (B-cells), which are precursors of antibody-secreting cells, and T-lymphocytes (T-cells).

### B-Lymphocytes

B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses (Paul, W. E., “Chapter 1: The immune system: an introduction,” Fundamental Immunology, 4th Edition, Ed. Paul, W. E., Lippicott-Raven Publishers, Philadelphia, (1999)).

Cross-linkage dependent B-cell activation requires that the antigen express multiple copies of the epitope complementary to the binding site of the cell surface receptors, because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes (Paul, W. E., “Chapter 1: The immune system: an introduction”, Fundamental Immunology, 4th Edition, Ed. Paul, W. E., Lippicott-Raven Publishers, Philadelphia, (1999)).

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4⁺ T-cells. The CD4⁺ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell (Paul, W. E., “Chapter 1: The immune system: an introduction, “Fundamental Immunology, 4th Edition, Ed. Paul, W. E., Lippicott-Raven Publishers, Philadelphia, (1999)).

During cognate help for antibody production, the CD40 ligand is transiently expressed on activated CD4⁺ T helper cells, and it binds to CD40 on the antigen-specific B cells, thereby transducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in pathogenic autoantibody production in human SLE patients (Desai-Mehta, A. et al., “Hyperexpression of CD40 ligand by B and T cells in human lupus and its role in pathogenic autoantibody production,” J. Clin. Invest. Vol. 97(9), 2063-2073, (1996)).

### T-Lymphocytes

T-lymphocytes derived from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on T cell expression of specific cell surface molecules and the secretion of cytokines (Paul, W. E., “Chapter 1: The immune system: an introduction”, Fundamental Immunology, 4th Edition, Ed. Paul, W. E., Lippicott-Raven Publishers, Philadelphia, (1999)).

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. While antibody and B-cell receptors evolved to bind to and to protect against microorganisms in extracellular fluids, T cells recognize antigens on the surface of other cells and mediate their functions by interacting with, and altering, the behavior of these antigen-presenting cells (APCs). There are three main types of APCs in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an APC that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the APC for long enough to become activated (“Chapter 24: The adaptive immune system,” Molecular Biology of the Cell, Alberts, B. et al., Garland Science, NY, (2002)).

T-cells are subdivided into two distinct classes based on the cell surface receptors they express. The majority of T cells express T cell receptors (TCR) consisting of α and β-chains. A small group of T cells express receptors made of γ and δ chains. Among the α/β T cells are two sub-lineages: those that express the coreceptor molecule CD4 (CD4⁺ T cells); and those that express CD8 (CD8⁺ T cells). These cells differ in how they recognize antigen and in their effector and regulatory functions.

CD4⁺ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells, particularly CD8⁺ T cells, can develop into cytotoxic T-lymphocytes (CTLs) capable of efficiently lysing target cells that express antigens recognized by the CTLs (Paul, W. E., “Chapter 1: The immune system: an introduction,” Fundamental Immunology, 4th Edition, Ed. Paul, W. E., Lippicott-Raven Publishers, Philadelphia, (1999)).

T cell receptors (TCRs) recognize a complex consisting of a peptide derived by proteolysis of the antigen bound to a specialized groove of a class II or class I MHC protein. CD4⁺ T cells recognize only peptide/class II complexes while CD8⁺ T cells recognize peptide/class I complexes (Paul, W. E., “Chapter 1: The immune system: an introduction,” Fundamental Immunology, 4th Edition, Ed. Paul, W. E., Lippicott-Raven Publishers, Philadelphia, (1999)).

The TCR's ligand (i.e., the peptide/MHC protein complex) is created within APCs. In general, class II MHC molecules bind peptides derived from proteins that have been taken up by the APC through an endocytic process. These peptide-loaded class II molecules are then expressed on the surface of the cell, where they are available to be bound by CD4⁺ T cells with TCRs capable of recognizing the expressed cell surface complex. Thus, CD4⁺ T cells are specialized to react with antigens derived from extracellular sources (Paul, W. E., “Chapter 1: The immune system: an introduction,” Fundamental Immunology, 4th Edition, Ed. Paul, W. E., Lippicott-Raven Publishers, Philadelphia, (1999)).

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally composed of nine amino acids in length, are bound into the class I MHC molecules and are brought to the cell surface, where they can be recognized by CD8⁺ T cells expressing appropriate receptors. This gives the T cell system, particularly CD8⁺ T cells, the ability to detect cells expressing proteins that are different from, or produced in much larger amounts than, those of cells of the remainder of the organism (e.g., viral antigens) or mutant antigens (such as active oncogene products), even if these proteins in their intact form are neither expressed on the cell surface nor secreted (Paul, W. E., “Chapter 1: The immune system: an introduction,” Fundamental Immunology, 4th Edition, Ed. Paul, W. E., Lippicott-Raven Publishers, Philadelphia, (1999)).

T cells can also be classified based on their function as helper T cells; T cells involved in inducing cellular immunity; suppressor T cells; and cytotoxic T cells.

### Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes, and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface. (Paul, W. E., “Chapter 1: The immune system: an introduction,” Fundamental Immunology, 4th Edition, Ed. Paul, W. E., Lippicott-Raven Publishers, Philadelphia (1999)).

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching of the Ig class being expressed, either depend or are enhanced by the actions of T cell-derived cytokines (Paul, W. E., “Chapter 1: The immune system: an introduction,” Fundamental Immunology, 4th Edition, Ed. Paul, W. E., Lippicott-Raven Publishers, Philadelphia, (1999)).

CD4⁺ T cells tend to differentiate into cells that principally secrete the cytokines IL-4, IL-5, IL-6, and IL-10 (T_{H}2 cells) or into cells that mainly produce IL-2, IFN-γ, and lymphotoxin (T_{H}1 cells). The T_{H}2 cells are very effective in helping B-cells develop into antibody-producing cells, whereas the T_{H}1 cells are effective inducers of cellular immune responses, involving enhancement of microbicidal activity of monocytes and macrophages, and consequent increased efficiency in lysing microorganisms in intracellular vesicular compartments. Although CD4⁺ T cells with the phenotype of T_{H}2 cells (i.e., IL-4, IL-5, IL-6 and IL-10) are efficient helper cells, T_{H}1 cells also have the capacity to be helpers (Paul, W. E., “Chapter 1: The immune system: an introduction, “Fundamental Immunology, 4th Edition, Ed. Paul, W. E., Lippicott-Raven Publishers, Philadelphia, (1999)).

### T Cell Involvement in Cellular Immunity Induction

T cells also may act to enhance the capacity of monocytes and macrophages to destroy intracellular microorganisms. In particular, interferon-gamma (IFN-γ) produced by helper T cells enhances several mechanisms through which mononuclear phagocytes destroy intracellular bacteria and parasitism including the generation of nitric oxide and induction of tumor necrosis factor (TNF) production. T_{H1} cells are effective in enhancing the microbicidal action, because they produce IFN-γ. In contrast, two of the major cytokines produced by T_{H2} cells, IL-4 and IL-10, block these activities (Paul, W. E., “Chapter 1: The immune system: an introduction,” Fundamental Immunology, 4th Edition, Ed. Paul, W. E., Lippicott-Raven Publishers, Philadelphia, (1999)).

### Regulatory T (Treg) Cells

Immune homeostasis is maintained by a controlled balance between initiation and downregulation of the immune response. The mechanisms of both apoptosis and T cell anergy (a tolerance mechanism in which the T cells are intrinsically functionally inactivated following an antigen encounter (Scwartz, R. H., “T cell anergy”, Annu. Rev. Immunol., Vol. 21: 305-334 (2003)) contribute to the downregulation of the immune response. A third mechanism is provided by active suppression of activated T cells by suppressor or regulatory CD4⁺ T (Treg) cells (Reviewed in Kronenberg, M. et al., “Regulation of immunity by self-reactive T cells”, Nature, Vol. 435: 598-604 (2005)). CD4⁺ Tregs that constitutively express the IL-2 receptor alpha (IL-2Rα) chain (CD4⁺ CD25⁺) are a naturally occurring T cell subset that are anergic and suppressive (Taams, L. S. et al., “Human anergic/suppressive CD4⁺ CD25⁺ T cells: a highly differentiated and apoptosis-prone population”, Eur. J. Immunol. Vol. 31: 1122-1131 (2001)). Depletion of CD4⁺ CD25⁺ Tregs results in systemic autoimmune disease in mice. Furthermore, transfer of these Tregs prevents development of autoimmune disease. Human CD4⁺ CD25⁺ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4⁺ CD25⁺ T cells can be split into suppressive (CD25^{high}) and nonsuppressive (CD25^{low}) cells, according to the level of CD25 expression. A member of the forkhead family of transcription factors, FOXP3, has been shown to be expressed in murine and human CD4⁺ CD25⁺ Tregs and appears to be a master gene controlling CD4⁺ CD25⁺ Treg development (Battaglia, M. et al., “Rapamycin promotes expansion of functional CD4⁺ CD25⁺ Foxp3⁺ regulator T cells of both healthy subjects and type 1 diabetic patients”, J. Immunol., Vol. 177: 8338-8347, (2006)).

### Cytotoxic T Lymphocytes

CD8⁺ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by granzymes, a series of enzymes produced by activated CTLs. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

### Lymphocyte Activation

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the APC.

### T-Memory Cells

Following the recognition and eradication of pathogens through adaptive immune responses, the vast majority (90-95%) of T cells undergo apoptosis with the remaining cells forming a pool of memory T cells, designated central memory T cells (TCM), effector memory T cells (TEM), and resident memory T cells (TRM) (Clark, R. A., “Resident memory T cells in human health and disease”, Sci. Transl. Med., 7, 269rv1, (2015)). CD45RA is expressed on naïve T cells, as well as the effector cells in both CD4 and CD8. After antigen experience, central and effector memory T cells gain expression of CD45RO and lose expression of CD45RA. Thus either CD45RA or CD45RO is used to generally differentiate the naïve from memory populations. CCR7 and CD62L are two other markers that can be used to distinguish central and effector memory T cells. Naïve and central memory cells express CCR7 and CD62L in order to migrate to secondary lymphoid organs. Thus, naïve T cells are CD45RA+CD45RO−CCR7+CD62L+, central memory T cells are CD45RA−CD45RO+CCR7+CD62L+, and effector memory T cells are CD45RA−CD45RO+CCR7−CD62L−.

Compared to standard T cells, these memory T cells are long-lived with distinct phenotypes such as expression of specific surface markers, rapid production of different cytokine profiles, capability of direct effector cell function, and unique homing distribution patterns. Memory T cells exhibit quick reactions upon re-exposure to their respective antigens in order to eliminate the reinfection of the offender and thereby restore balance of the immune system rapidly. Increasing evidence substantiates that autoimmune memory T cells hinder most attempts to treat or cure autoimmune diseases (Clark, R. A., “Resident memory T cells in human health and disease”, Sci. Transl. Med., Vol. 7, 269rv1, (2015)).

II. Artificial Antigen Presenting Cells (aAPCs)

The present disclosure features erythroid cells (e.g., enucleated erythroid cells) and enucleated cells that are engineered to activate or suppress T cells. In some embodiments an enucleated cell is an erythroid cell, for example, that has lost its nucleus through differentiation from an erythrocyte precursor cell. It will be understood, however, that not all enucleated cells are erythroid cells and, accordingly, enucleated cells encompassed herein can also include, e.g., platelets. In some embodiments, enucleated cells are not platelets and are therefore platelet free enucleated cells. In certain aspects of the disclosure, the enucleated cell is a reticulocyte or erythrocyte (fully mature red blood cell (RBC)). Erythrocytes offer a number of advantages over other cells, including being non-autologous (e.g., substantially lack major histocompatibility complex (MHC)), having longer circulation time in a subject (e.g. greater than 30 days), and being amenable to production in large numbers.

The skilled artisan would appreciate, based upon the disclosure provided herein, that numerous immunoregulatory molecules can be used to produce an almost limitless variety of aAPCs once armed with the teachings provided herein. That is, there is extensive knowledge in the art regarding the events and molecules involved in activation and induction of T cells.

In some aspects, the present disclosure provides an engineered erythroid cell or an enucleated cell comprising an exogenous polypeptide, e.g., comprising or presenting the exogenous polypeptide on the cell surface. Exogenous polypeptides of the present disclosure include, but are not limited to, exogenous antigenic polypeptides, exogenous antigen-presenting polypeptides, exogenous costimulatory polypeptides, exogenous coinhibitory polypeptides, exogenous metabolic modulating polypeptides, and exogenous Treg costimulatory polypeptides.

### Exogenous Antigenic Polypeptides

An exogenous antigenic polypeptide is a polypeptide that is capable of inducing an immune response. In some embodiments, an exogenous antigenic polypeptide is a polypeptide that, by inducing an immune response, inhibits a cancer, e.g., reduces or alleviates a cause or symptom of a cancer, or improves a value for a parameter associated with the cancer. In some embodiments, an exogenous antigenic polypeptide is a polypeptide that, by inducing an immune response, inhibits an infectious disease, e.g., reduces or alleviates a cause or symptom of an infectious disease, or improves a value for a parameter associated with the infectious disease. In some embodiments, an exogenous antigenic polypeptide is a polypeptide that, by inducing an immune response, inhibits an autoimmune disease, e.g., reduces or alleviates a cause or symptom of an autoimmune disease, or improves a value for a parameter associated with the autoimmune disease.

In certain embodiments, the exogenous antigenic polypeptide comprises or consist of an antigenic polypeptide selected from Table 1, or a fragment or variant thereof, or an antibody molecule thereto.

TABLE 1

| | | | | | | | | |
|---|---|---|---|---|---|---|---|---|
| | | | | | | | Lymphocyte | |
| | | | HLA Frequency | | | | Stimulation | |
| Gene/Protein | Tumor | HLA | (%) | Peptide | SEQ ID NO: | Position | Method | References |

MUTATIONS

| | | | | | | | | |
|---|---|---|---|---|---|---|---|---|
| ABL-BCR alb-b3 | | A2 | | FVEHDDESPGL | 16 | | | Wagner, 2003 |
| (b2a2) | | | | | | | | |
| Mutation | | | | | | | | |
| ABL-BCR alb-b4 | | A3 | | FVEHDDESPGL | 16 | | | Wagner, 2004 |
| (b3a2) | | | | | | | | |
| Mutation | | | | | | | | |
| PPP1R3B | melanoma | A1 | 26 | YTDFHCQYV | 17 | 172-180 | autologous | Robbins, 2013 |
| | | | | | | | tumor | |
| alpha-actinin-4 | lung | A2 | 44 | FIASNGVKLV | 18 | 118-127 | autologous | Echchakir, 2001 |
| | carcinoma | | | | | | tumor cells | |
| ARTC1 | melanoma | DR1 | 18 | YSVYFNLPADTIYTNh | 19 | | autologous | Wang, 2005 |
| | | | | | | | tumor cells | |
| CASP-8 | head and neck | B35 | 20 | FPSDSWCYF | 20 | 476-484 | autologous | Mandruzzato, 1997 |
| | squamous cell | | | | | | tumor cells | |
| | carcinoma | | | | | | | |
| beta-catenin | melanoma | A24 | 20 | SYLDSGIHF | 21 | 29-37 | autologous | Robbins, 1996 |
| | | | | | | | tumor cells | |
| Cdc27 | melanoma | DR4 | 24 | FSWAMDLDPKGAe | 22 | 760-771 | autologous | Wang 1999b |
| | | | | | | | tumor cells | |
| CDK4 | melanoma | A2 | 44 | ACDPHSGHFV | 23 | 23-32 | autologous | Wolfel, 1995 |
| | | | | | | | tumor cells | |
| CDK12 | melanoma | A11 | 13 | CILGKLFTK | 24 | 924-932 | autologous | Robbins, 2013 |
| | | | | | | | tumor cells | |
| CDKN2A | melanoma | A11 | 13 | AVCPWTWLRg | 25 | 125-133 | autologous | Huang, 2004 |
| | | | | | | (p14ARF-ORF3) | tumor cells | |
| CLPP | melanoma | A2 | 44 | ILDKVLVHL | 26 | 240-248 | autologous | Corbiere, 2011 |
| | | | | | | | tumor cells | |
| COA-1 | colorectal | DR4 | 24 | TLYQDDTLTLQAAGe | 27 | 447-460 | autologous | Maccalli, 2003 |
| | | | | | | | tumor cells | |
| | carcinoma | DR13 | 19 | TLYQDDTLTLQAAGe | 27 | 447-460 | autologous | Maccalli, 2003 |
| | | | | | | | tumor cells | |
| CSNK1A1 | melanoma | A2 | 44 | GLFGDIYLA | 28 | 26-34 | autologous | Robbins, 2013 |
| | | | | | | | tumor cells | |
| EFTUD2 | melanoma | A3 | 22 | KILDAVVAQK | 29 | 668-677 | autologous | Lennerz, 2005 |
| | | | | | | | tumor cells | |
| Elongation | lung | A68 | 8 | ETVSEQSNV | 30 | 581-589 | autologous | Hogan, 1998 |
| factor 2 | squamous CC | | | | | | tumor cells | |
| FN1 | melanoma | DR2 | 25 | MIFEKHGFRRTTPP | 31 | 2050-2063 | autologous | Wang, 2002 |
| | | | | | | | tumor cells | |
| GAS7 | melanoma | A2 | 44 | SLADEAEVYL | 32 | 141-150 | autologous | Robbins, 2013 |
| | | | | | | | tumor cells | |
| GPNMB | melanoma | A3 | 22 | TLDWLLQTPK | 33 | 179-188 | autologous | Lennerz, 2005 |
| | | | | | | | tumor cells | |
| HAUS3 | melanoma | A2 | 44 | ILNAMIAKIj | 34 | 154-162 | autologous | Robbins, 2013 |
| | | | | | | | tumor cells | |
| HSDL1 | ovarian cancer | Cw14 | 4 | CYMEAVAL | 35 | 20-27 | autologous | Wick, 2013 |
| | | | | | | | tumor cells | |
| LDLR- | melanoma | DR1 | 18 | WRRAPAPGA | 36 | 315-323 | autologous | Wang, 1999a |
| fucosyltransferase | | | | PVTWRRAPA | 37 | 312-320 | tumor cells | |
| AS fusion | | | | | | | | |
| protein | | | | | | | | |
| HLA-A2d | renal cell | | | | | | autologous | Brandle, 1996 |
| | carcinoma | | | | | | tumor cells | |
| HLA-A11d | melanoma | | | | | | autologous | Huang, 2004 |
| | | | | | | | tumor cells | |
| HLA-A0201- | | A2 | | CVEWLRIYLENG | 38 | | | Novellino, L. 2004 |
| R170I | | | | | | | | |
| hsp70-2 | renal cell | A2 | 44 | SLFEGIDIYT | 39 | 286-295 | autologous | Gaudin, 1999 |
| | carcinoma | | | | | | tumor cells | |
| | bladder tumor | B44 | 21 | AEPINIQTW | 40 | 262-270 | autologous | Gueguen, 1998 |
| | | | | | | | tumor cells | |
| IgGH | | A2 | | LMISRTPEV | 41 | | | Belle S, 2008 |
| MART2 | melanoma | A1 | 26 | FLEGNEVGKTY | 42 | 446-455 | autologous | Kawakami, 2001 |
| | | | | | | | tumor cells | |
| MATN | melanoma | A11 | 13 | KTLTSVFQK | 43 | 226-234 | autologous | Robbins, 2013 |
| | | | | | | | tumor cells | |
| k-ras | non-small cell | A2 | 44 | FLDEFMEGV | 44 | 224-232 | autologous | Karanikas, 2001 |
| | lung | | | | | | tumor cells | |
| | carcinoma | A2 | | KLVVVGAVGV | 45 | | | Kubuschok, 2006 |
| | | A2 | | LVVVGAVGV | 46 | | | Kubuschok, 2006 |
| | | B35 | | VVVGAVGVG | 47 | | | Gjertsen, 1997 |
| MUM-1f | melanoma | B44 | 21 | EEKLIVVLF | 48 | 30-38 | autologous | Coulie, 1995 |
| | | | | | | | tumor cells | |
| MUM-2 | melanoma | B44 | 21 | SELFRSGLDSY | 49 | 123-133 | autologous | Chiari, 1999 |
| | | Cw6 | 18 | FRSGLDSYV | 50 | 126-134 | tumor cells | |
| MUM-3 | melanoma | A68 | 8 | EAFIQPITR | 51 | 322-330 | autologous | Baurain, 2000 |
| | | | | | | | tumor cells | |
| neo-PAP | melanoma | DR7 | 25 | RVIKNSIRLTLe | 52 | 724-734 | autologous | Topalian, 2002 |
| | | | | | | | tumor cells | |
| NFYC | lung | B52 | 5 | QQITKTEV | 53 | 275-282 | autologous | Takenoyama, 2006 |
| | squamous cell | | | | | | tumor cells | |
| | carcinoma | | | | | | | |
| OS-9 | melanoma | B44 | 21 | KELEGILLL | 54 | 438-446 | autologous | Vigneron, 2002 |
| | | | | | | | tumor cells | |
| PTPRK | melanoma | DR10 | 3 | PYYFAAELPPRNLPEE | 55 | 667-682 | autologous | Novellino, 2003 |
| | | | | | | | tumor cells | |
| N-ras | melanoma | A1 | 26 | ILDTAGREEY | 56 | 55-64 | autologous | Linard, 2002 |
| | | | | | | | tumor cells | |
| BRAF600 | melanoma | B7 | 17 | RPHVPESAF | 57 | 329-337 | autologous | Lennerz, 2005 |
| | | | | | | | tumor cells | |
| SIRT2 | melanoma | A3 | 22 | KIFSEVTLK | 58 | 192-200 | autologous | Lennerz, 2005 |
| | | | | | | | tumor cells | |
| SNRPD1 | melanoma | B38 | 5 | SHETVIIEL | 59 | 19-Nov | autologous | Lennerz, 2005 |
| | | | | | | | tumor cells | |
| Triosephosphate | melanoma | DR1 | 18 | GELIGILNAAKVPAD | 60 | 23-37 | autologous | Pieper, 1999 |
| isomerase | | | | | | | tumor cells | |
| Myosin class I | melanoma | A3 | 22 | KINKNPKYK | 61 | 911-919 | expansion of TIL | Zorn, 1999 |
| | | | | | | | with IL-2 | |
| BCR-ABL fusion | | A2 | | KLSEQESLL | 62 | | | Buzyn, 1997 |
| | | A2 | | MLTNSCVKL | 63 | | | Buzyn, 1997 |
| | | A2 | | FMVELVEGA | 64 | | | Buzyn, 1997 |
| | | A2 | | GVRGRVEEI | 65 | | | Volpe, 2007 |
| BCR-ABL fusion | chronic | A2 | 44 | SSKALQRPV | 66 | 926-934 | peptide | Yotnda, 1998a |
| protein (b3a2) | myeloid | A3 | | ATGFKQSSK | 67 | | | Greco, 1996 |
| | leukemia | A3 | | HSATGFKQSSK | 68 | | | Bocchia, 1996 |
| | | A3 | | KQSSKALQR | 69 | | | Greco, 1996 |
| | | A11 | | HSATGFKQSSK | 68 | | | Bocchia, 1996 |
| | | B8 | 14 | GFKQSSKAL | 70 | 922-930 | peptide | Yotnda, 1998a |
| | | DR4 | 24 | ATGFKQSSKALQRP | 71 | 920-936 | peptide | ten Bosch, 1996 |
| | | | | VAS | | | | |
| | | DR9 | 3 | ATGFKQSSKALQRP | 71 | 920-936 | peptide | Makita, 2002 |
| | | | | VAS | | | | |
| B-RAF | melanoma | A2 | | LATEKSRWS | 72 | | | Somasundaram, |
| | | | | | | | | 2006 |
| | | A2 | | LATEKSRWSG | 73 | | | Somasundaram, |
| | | | | | | | | 2006 |
| | | A33 | | FGLATEKSR | 74 | | | Andersen, 2004 |
| | | B27 | | GDFGLATEK | 75 | | | Andersen, 2004 |
| | | DR4 | 24 | EDLTVKIGDFGLATE | 76 | 586-614 | peptide | Sharkey, 2004 |
| | | | | KSRWSGSHQFEQLS | | | | |
| CASP-5 | colorectal, | A2 | 44 | FLIIWQNTM | 77 | 67-75 | peptide | Schwitalle, 2004 |
| | gastric, and | | | | | | | |
| | endometrial | | | | | | | |
| | carcinoma | | | | | | | |
| dek-can fusion | myeloid | DR53 | 49 | TMKQICKKEIRRLHQY | 78 | 342-357 | peptide | Makita, 2002 |
| protein | leukemia | | | | | | | |
| ETV6-AML1 | acute | A2 | 44 | RIAECILGMi | 79 | 334-342 | peptide | Yotnda, 1998b |
| fusion protein | lymphoblastic | DP5 | 3 | IGRIAECILGMNPSR | 80 | 332-346 | peptide | Yun, 1999 |
| | leukemia | DP17 | 1 | IGRIAECILGMNPSR | 80 | 332-346 | peptide | Yun, 1999 |
| FLT3-ITD | acute | A1 | 26 | YVDFREYEYY | 81 | 591-600 | peptide | Graf, 2007 |
| | myelogenous | | | | | | | |
| | leukemia | | | | | | | |
| FNDC3B | chronic | A2 | 44 | VVMSWAPPV | 82 | 292-300 | peptide | Rajasagi, 2004 |
| | lymphocytic | | | | | | | |
| | leukemia | | | | | | | |
| OGT | colorectal | A2 | 44 | SLYKFSPFPLg | 83 | 28-37 | peptide | Ripberger, 2003 |
| | carcinoma | | | | | | | |
| p53 | head and neck | A2 | 44 | VVPCEPPEV | 84 | 217-225 | peptide | Ito, 2007 |
| | squamous cell | | | | | | | |
| | carcinoma | | | | | | | |
| pml-RARalpha | promyelocytic | DR11 | 25 | NSNHVASGAGEAAI | 85 | | peptide | Gambacorti, 1993 |
| fusion protein | leukemia | | | ETQSSSSEEIV | | | | |
| PAX-FKHR | | B7 | | SPANSIRHNL | 86 | | | van den Broeke, |
| fusion | | | | | | | | 2006 |
| | | B7 | | SPQNSIRHNL | 87 | | | van den Broeke, |
| | | | | | | | | 2006 |
| PRDX5 | melanoma | A2 | 44 | LLLDDLLVSI | 88 | 163-172 | peptide | Sensi, 2005 |
| | | A2 | | AMAPIKVRL | 89 | | | Sensi, 2009 |
| K-ras | pancreatic | B35 | 20 | VVVGAVGVG | 47 | 15-Jul | peptide | Gjertsen, 1997 |
| | adenocarcinoma | | | | | | | |
| SYT-SSX1 or - | sarcoma | A24 | | GYDQIMPKK | 90 | | | Sato, 2002; Ida, |
| SSX2 fusion | | | | | | | | 2004 |
| protein | | A24 | | GYDQIMPKI | 91 | | | Ida, 2004 |
| | | B7 | 17 | QRPYGYDQIM | 92 | 402-410 (SYT) | peptide | Worley, 2001 |
| KIAAO205 | mutation | B44 | 21 | AEPINIQTW | 40 | | peptide | Gueguen, 1998 |
| ME1 | mutation | A2 | 44 | FLDEFMEGV | 44 | | peptide | Karanikas, 2001 |
| EGFRvIII | Mutation | A2 | 44 | LEEKKGNYV | 93 | | peptide | Wu, 2006 |
| TGF-betaRII | colorectal | A2 | 44 | RLSSCVPVAg | 94 | 131-139 | peptide | Linnebacher, 2001 |
| | carcinoma | A2 | 44 | RLSSCVPVAg | 94 | 111- | peptide | Lu, 2013 |
| | | | | | | 119(p16INK4a- | | |
| | | | | | | ORF3) | | |
| | | | | | | 111-112 (SSX2) | | |

TUMOR-SPECIFIC

| | | | | | | | |
|---|---|---|---|---|---|---|---|
| BAGE-1 | Cw16 | 7 | AARAVFLAL | 95 | 10-Feb | autologous | Boel, 1995 |
| | | | | | | tumor cells | |
| D393-CD20n | DR4 | 24 | KPLFRRMSSLELVIA | 96 | 28-42 | peptide | Vauchy, 2015 |
| Cyclin-A1 | A2 | 44 | FLDRFLSCM | 97 | 227-235 | peptide | Ochsenreither, |
| | | | | | | | 2012 |
| | A2 | 44 | SLIAAAAFCLA | 98 | 341-351 | peptide | Ochsenreither, |
| | | | | | | | 2012 |
| GAGE-1,2,8 | Cw6 | 18 | YRPRPRRY | 99 | 16-Sep | autologous | Van den Eynde, |
| | | | | | | tumor cells | 1995 |
| GAGE-3,4,5,6,7 | A29 | 6 | YYWPRPRRY | 100 | 18-Oct | autologous | De Backer, 1999 |
| | | | | | | tumor cells | |
| GnTVf | A2 | 44 | VLPDVFIRC(V) | 101 | intron | autologous | Guilloux, 1996 |
| | | | | | | tumor cells | |
| GPC3 | A2 | | FLAELAYDL | 102 | | | O'beirne, 2010 |
| HERV-E | A11 | | ATFLGSLTWK | 103 | | | Takahashi, 2008 |
| HERV-K-MEL | A2 | 44 | MLAVISCAV | 104 | 9-Jan | autologous | Schiavetti, 2002 |
| | | | | | | tumor cells | |
| KK-LC-1 | B15 | 13 | RQKRILVNL | 105 | 76-84 | autologous | Fukuyama, 2006 |
| | | | | | | tumor cells | |
| KM-HN-1 | A24 | 20 | NYNNFYRFL | 106 | 196-204 | peptide | Monji, 2004 |
| | A24 | 20 | EYSKECLKEF | 107 | 499-508 | peptide | Monji, 2004 |
| | A24 | 20 | EYLSLSDKI | 108 | 770-778 | peptide | Monji, 2004 |
| LAGE-1 | A2 | 44 | MLMAQEALAFL | 109 | ORF2 | autologous | Aarnoudse, 1999 |
| | | | | | (1-11) | tumor cells | |
| | A2 | 44 | SLLMWITQC | 110 | 157-165 | peptide | Rimoldi, 2000 |
| | A31 | 5 | LAAQERRVPR | 111 | ORF2 | autologous | Wang, 1998 |
| | | | | | (18-27) | tumor cells | |
| | A68 | 8 | ELVRRILSR | 112 | 103-111 | adenovirus- | Sun, 2006 |
| | | | | | | dendritic cells | |
| | B7 | 17 | APRGVRMAV | 113 | ORF2 | adenovirus-APC | Slager, 2004b |
| | | | | | (46-54) | | |
| | DP4 | 75 | SLLMWITQCFLPVF | 114 | 157-170 | peptide | Zeng, 2001 |
| | DR3 | 21 | QGAMLAAQERRVP | 115 | ORF2 | protein | Slager, 2004a |
| | | | RAAEVPR | | (14-33) | | |
| | DR4 | 24 | AADHRQLQLSISSCL | 116 | 139-156 | protein | Jager, 2000 |
| | | | QQL | | | | |
| | DR11 | 25 | CLSRRPWKRSWSAG | 117 | ORF2 | peptide | Slager, 2003 |
| | | | SCPGMPHL | | (81-102) | | |
| | DR12 | 5 | CLSRRPWKRSWSAG | 117 | ORF2 | peptide | Slager, 2003 |
| | | | SCPGMPHL | | (81-102) | | |
| | DR13 | 19 | ILSRDAAPLPRPG | 118 | 108-120 | autologous | Wang, 2004 |
| | | | | | | tumor cells | |
| | DR15 | 20 | AGATGGRGPRGAGA | 119 | 37-50 | protein | Hasegawa, 2006 |
| LY6K | A24 | 20 | RYCNLEGPPI | 120 | 119-128 | peptide | Suda, 2007 |
| | DP5 | 3 | KWTEPYCVIAAVKIF | 121 | 61-84 | peptide | Tomita, 2014 |
| | | | PRFFMVAKQ | | | | |
| | DR15 | 20 | KCCKIRYCNLEGPPI | 122 | 114-133 | peptide | Tomita, 2014 |
| | | | NSSVF | | | | |
| MAGE-A1 | A1 | 26 | EADPTGHSY | 123 | 161-169 | autologous | Traversari, 1992 |
| | | | | | | tumor cells | |
| | A*0201 | | YLEYRQVPV | 124 | | | |
| | | | YLEYRQVPD | 125 | | | |
| | A2 | 44 | KVLEYVIKV | 126 | 278-286 | peptide | Ottaviani, 2005 |
| | | | | | | | Pascolo, 2001 |
| | A3 | 22 | SLFRAVITK | 127 | 96-104 | poxvirus- | Chaux, 1999a |
| | | | | | | dendritic cellsc | |
| | A24 | | NYKHCFPEI | 128 | | | Fujie, 1999 |
| | A68 | 8 | EVYDGREHSA | 129 | 222-231 | poxvirus- | Chaux, 1999a |
| | | | | | | dendritic cells | |
| | B7 | 17 | RVRFFFPSL | 130 | 289-298 | poxvirus- | Luiten, 2000a |
| | | | | | | dendritic cells | |
| | B35 | 20 | EADPTGHSY | 123 | 161-169 | poxvirus- | Luiten, 2000b |
| | | | | | | dendritic cells | |
| | B37 | 3 | REPVTKAEML | 131 | 120-129 | autologous | Tanzarella, 1999 |
| | | | | | | tumor cells | |
| | B44 | 21 | KEADPTGHSY | 132 | 160-169 | poxvirus- | Stroobant, 2012 |
| | | | | | | dendritic cells | |
| | B53 | 2 | DPARYEFLW | 133 | 258-266 | poxvirus- | Chaux, 1999a |
| | | | | | | dendritic cells | |
| | B57 | 8 | ITKKVADLVGF | 134 | 102-112 | ALVAC-dendritic | Corbiere, 2004 |
| | | | | | | cells | |
| | Cw2 | 10 | SAFPTTINF | 135 | 62-70 | poxvirus- | Chaux, 1999a |
| | | | | | | dendritic cells | |
| | Cw3 | 17 | SAYGEPRKL | 136 | 230-238 | poxvirus- | Chaux, 1999a |
| | | | | | | dendritic cells | |
| | Cw7 | 41 | RVRFFFPSL | 130 | 289-298 | peptide | Goodyear, 2011 |
| | Cw16 | 7 | SAYGEPRKL | 136 | 230-238 | autologous | van der Bruggen, |
| | | | | | | tumor cells | 1994a |
| | DP4 | 75 | TSCILESLFRAVITK | 137 | 90-104 | peptide | Wang, 2007 |
| | DP4 | 75 | PRALAETSYVKVLEY | 138 | 268-282 | peptide | Wang, 2007 |
| | DR13 | 19 | FLLLKYRAREPVTKAE | 139 | 112-127 | protein | Chaux, 1999b |
| | DR15 | 20 | EYVIKVSARVRF | 140 | 281-292 | protein | Chaux, 2001 |
| MAGE-A2 | A2 | 44 | YLQLVFGIEV | 141 | 157-166 | peptide | Kawashima, 1998 |
| | A2 | | YLEYRQVPV | 124 | | | Graff-Dubois, 2002 |
| | A2 | | LVHFLLLKY | 142 | | | Bredenbeck. 2005 |
| | A2 | | KMVELVHFL | 143 | | | Visseren, 1997 |
| | A2 | | LVQENYLEY | 144 | | | Bredenbeck. 2005 |
| | A24 | 20 | EYLQLVFGI | 145 | 156-164 | peptide | Tahara, 1999 |
| | B37 | 3 | REPVTKAEML | 131 | 127-136 | autologous | Tanzarella, 1999 |
| | | | | | | tumor cells | |
| | Cw7 | 41 | EGDCAPEEK | 146 | 212-220 | lentivirus- | Breckpot, 2004 |
| | | | | | | dendritic cells | |
| | DR13 | 19 | LLKYRAREPVTKAE | 147 | 121-134 | protein | Chaux, 1999b |
| MAGE-A3 | A1 | 26 | EVDPIGHLY | 148 | 168-176 | autologous | Gaugler, 1994 |
| | | | | | | tumor cells | |
| | A2 | 44 | FLWGPRALVd | 149 | 271-279 | peptide | van der Bruggen, |
| | | | | | | | 1994b |
| | A2 | 44 | KVAELVHFL | 150 | 112-120 | peptide | Kawashima, 1998 |
| | A2 | | YLEYRQVPV | 124 | | | Graff-Dubois, 2002 |
| | A2 | | LVFGIELMEV | 151 | | | Keogh, 2001 |
| | A24 | | IMPKAGLLI | 152 | | | Tanaka, 2000 |
| | A24 | 20 | TFPDLESEF | 153 | 97-105 | peptide | Oiso, 1999 |
| | A24 | 20 | VAELVHFLL | 154 | 113-121 | peptide | Miyagawa, 2006 |
| | B18 | 6 | MEVDPIGHLY | 155 | 167-176 | adeno-dendritic | Bilsborough, 2002 |
| | | | | | | cells | |
| | | | YLEYRQVPG | 156 | | | |
| | B35 | 20 | EVDPIGHLY | 148 | 168-176 | poxvirus- | Schultz, 2001 |
| | | | | | | dendritic cells | |
| | B37 | 3 | REPVTKAEML | 131 | 127-136 | autologous | Tanzarella, 1999 |
| | | | | | | tumor cells | |
| | B40 | 6 | AELVHFLLLi | 157 | 114-122 | adeno-dendritic | Schultz, 2002 |
| | | | | | | cells | |
| | B44 | 21 | MEVDPIGHLY | 155 | 167-176 | peptide | Herman, 1996 |
| | B52 | 5 | WQYFFPVIF | 158 | 143-151 | retrovirus- | Russo, 2000 |
| | | | | | | dendritic cellsh | |
| MAGE-A3 | Cw7 | 41 | EGDCAPEEK | 146 | 212-220 | lentivirus- | Breckpot, 2004 |
| | | | | | | dendritic cells | |
| | DP4 | 75 | KKLLTQHFVQENYLEY | 159 | 243-258 | protein | Schultz, 2000 |
| | DP4 | 75 | RKVAELVHFLLLKYR | 160 | 111-125 | peptide | Cesson, 2011 |
| | DQ6 | 63 | KKLLTQHFVQENYLEY | 159 | 243-258 | peptide | Schultz, 2004 |
| | DR1 | 18 | ACYEFLWGPRALVE | 161 | 267-282 | protein | Zhang, 2003 |
| | | | TS | | | | |
| | DR4 | 24 | RKVAELVHFLLLKYR | 160 | 111-125 | peptide | Cesson, 2010 |
| | DR4 | 24 | VIFSKASSSLQL | 162 | 149-160 | peptide | Kobayashi, 2001 |
| | DR7 | 25 | VIFSKASSSLQL | 162 | 149-160 | peptide | Kobayashi, 2001 |
| | DR7 | 25 | VFGIELMEVDPIGHL | 163 | 161-175 | peptide | Cesson, 2011 |
| | DR11 | 25 | GDNQIMPKAGLLIIV | 164 | 191-205 | peptide | Consogno, 2003 |
| | DR11 | 25 | TSYVKVLHHMVKISG | 165 | 281-295 | protein | Manici, 1999 |
| | DR13 | 19 | RKVAELVHFLLLKYRA | 166 | 111-126 | protein | Chaux, 1999b |
| | DR13 | 19 | FLLLKYRAREPVTKAE | 139 | 119-134 | protein | Chaux, 1999b |
| MAGE-A4 | A1 | 26 | EVDPASNTYJ | 167 | 169-177 | peptide after | Kobayashi, 2003 |
| | | | | | | tetramer sorting | |
| | A2 | | YLEYRQVPV | 124 | | | Graff-Dubois, 2002 |
| | A2 | 44 | GVYDGREHTV | 168 | 230-239 | adeno-dendritic | Duffour, 1999 |
| | | | | | | cells | |
| | A24 | 20 | NYKRCFPVI | 169 | 143-151 | peptide | Miyahara, 2005 |
| | | | | | | | Ottaviani, 2006 |
| | B37 | 3 | SESLKMIF | 170 | 156-163 | poxvirus- | Zhang, 2002 |
| | | | | | | dendritic cells | |
| MAGE-A5 | | | | | | | |
| MAGE-A6 | A34 | 1 | MVKISGGPR | 171 | 290-298 | autologous | Zorn, 1999 |
| | | | | | | tumor cells | |
| | B35 | 20 | EVDPIGHVY | 172 | 168-176 | autologous | Benlalam, 2003 |
| | | | | | | tumor cells | |
| | B37 | 3 | REPVTKAEML | 131 | 127-136 | autologous | Tanzarella, 1999 |
| | | | | | | tumor cells | |
| | Cw7 | 41 | EGDCAPEEK | 146 | 212-220 | lentivirus- | Breckpot, 2004 |
| | | | | | | dendritic cells | |
| | Cw16 | 7 | ISGGPRISY | 173 | 293-301 | autologous | Vantomme, 2003 |
| | | | | | | tumor cells | |
| | | | YLEYRQVPG | 156 | | | |
| | DR13 | 19 | LLKYRAREPVTKAE | 147 | 121-134 | protein | Chaux, 1999b |
| MAGE-A7 | | | | | | | |
| MAGE-A8 | A2 | | KVAELVRFL | 174 | | | Bar-Haim, 2004 |
| | A2 | | GLMDVQIPT | 175 | | | Bar-Haim, 2004 |
| MAGE-A9 | A2 | 44 | ALSVMGVYV | 176 | 223-231 | peptide | Oehlrich, 2005 |
| MAGE-A10 | A2 | 44 | GLYDGMEHLI | 177 | 254-262 | autologous | Huang, 1999 |
| | | | | | | tumor cells | |
| | A2 | | YLEYRQVPG | 156 | | | Graff-Dubois, 2002 |
| | A2 | | SLLKFLAKV | 178 | | | Jia, ZC, 2011 |
| | B53 | 2 | DPARYEFLW | 133 | 290-298 | poxvirus- | Chaux, 1999a |
| | | | | | | de]ndritic cells | |
| MAGE-A11 | | | | | | | |
| MAGE-A12m | A2g | 44 | FLWGPRALVe | 179 | 271-279 | peptide | van der Bruggen, |
| | | | | | | | 1994b |
| | A2 | | YLEYRQVPV | 124 | | | Graff-Dubois, 2002 |
| | Cw7 | 41 | VRIGHLYIL | 180 | 170-178 | autologous | Heidecker, 2000 |
| | | | | | | tumor cells | Panelli, 2000 |
| | Cw7 | 41 | EGDCAPEEK | 146 | 212-220 | lentivirus- | Breckpot, 2004 |
| | | | | | | dendritic cells | |
| | DP4 | 75 | REPFTKAEMLGSVIR | 181 | 127-141 | peptide | Wang, 2007 |
| | | | YLEYRQVPG | 156 | | | |
| | DR13 | 19 | AELVHFLLLKYRAR | 182 | 114-127 | protein | Chaux, 1999b |
| MAGE-C1 | A2 | 44 | ILFGISLREV | 183 | 959-968 | peptide | Anderson, 2011 |
| | A2 | 44 | KVVEFLAML | 184 | 1083-1091 | peptide | Anderson, 2011 |
| | DQ6 | 63 | SSALLSIFQSSPE | 185 | 137-149 | peptide | Nuber, 2010 |
| | DQ6 | 63 | SFSYTLLSL | 186 | 450-458 | peptide | Nuber, 2010 |
| | DR15 | 20 | VSSFFSYTL | 187 | 779-787 | peptide | Nuber, 2010 |
| MAGE-C2 | A2 | 44 | LLFGLALIEV | 188 | 191-200 | autologous | Ma, 2004 |
| | | | | | | tumor cells | |
| | A2 | 44 | ALKDVEERV | 189 | 336-344 | autologous | Ma, 2004 |
| | | | | | | tumor cells | |
| | | | TLDEKVAELV | 190 | | | Xing, 2008 |
| | | | KVLEFLAKL | 191 | | | Xing, 2008 |
| | | | FLAKLNNTV | 192 | | | Li, 2005 |
| | | | VIWEVLNAV | 193 | | | Xing, 2008 |
| | B44 | 21 | SESIKKKVL | 194 | 307-315 | autologous | Godelaine, 2007 |
| | | | | | | tumor cells | |
| | B57 | 8 | ASSTLYLVF | 195 | 42-50 | autologous | Ma, 2011 |
| | | | | | | tumor cells | |
| | DR15 | 20 | SSTLYLVFSPSSFST | 196 | 43-57 | peptide | Wen, 2011 |
| MAGE-n | A2 | | FLWGPRALA | 197 | | | Dong, 2004 |
| | A2 | | QLVFGIEVV | 198 | | | Dong, 2004 |
| mucink | | | PDTRPAPGSTAPPA | 199 | | transfected B | Jerome, 1993 |
| | | | HGVTSA | | | cells | |
| NA88-A | B13 | 6 | QGQHFLQKV | 200 | | tumor- | Moreau-Aubry, |
| | | | | | | infiltrating | 2000 |
| | | | | | | lymphocytes | |
| NY-ESO-1/ | A2 | 44 | SLLMWITQC | 110 | 157-165 | autologous | Jager, 1998 |
| LAGE-2 | | | | | | tumor cells | Chen, 2000 |
| | | | | | | | Valmori, 2000 |
| | A2 | 44 | MLMAQEALAFL | 109 | ORF2 | autologous | Aarnoudse, 1999 |
| | | | | | (1-11) | tumor cells | |
| | A2 | | SLLMWITQCFL | 201 | | | Jager, 1998 |
| | A2 | | QLSLLMWIT | 202 | | | Jager, 1998 |
| | A24 | | LLMWITQCF | 203 | | | Yamaguchi, 2004 |
| | A24 | 20 | YLAMPFATPME | 204 | 91-101 | peptide | Eikawa, 2013 |
| NY-ESO-1/ | A31 | 5 | ASGPGGGAPR | 205 | 53-62 | autologous | Wang, 1998 |
| LAGE-2 | | | | | | tumor cells | |
| | A31 | 5 | LAAQERRVPR | 111 | ORF2 | autologous | Wang, 1998 |
| | | | | | (18-27) | tumor cells | |
| | A68 | 8 | TVSGNILTIR | 206 | 127-136 | mRNA- | Matsuzaki, 2008 |
| | | | | | | transfected cells | |
| | B7 | 17 | APRGPHGGAASGL | 207 | 60-72 | peptide | Ebert, 2009 |
| | B35 | 20 | MPFATPMEAEL | 208 | 94-104 | peptide | Eikawa, 2013 |
| | B49 | | KEFTVSGNILTI | 209 | 124-135 | mRNA- | Knights, 2009 |
| | | | | | | transfected cells | |
| | B51 | 12 | MPFATPMEA | 210 | 94-102 | adenovirus-APC | Jager, 2002 |
| | B52 | 5 | FATPMEAEL | 211 | 96-104 | peptide | Eikawa, 2013 |
| | C12 | 12 | FATPMEAELAR | 212 | 96-106 | peptide | Eikawa, 2013 |
| | Cw3 | 17 | LAMPFATPM | 213 | 92-100 | adenovirus- | Gnjatic, 2000 |
| | | | | | | PBMC | |
| | Cw6 | 18 | ARGPESRLL | 214 | 80-88 | adenovirus- | Gnjatic, 2000 |
| | | | | | | PBMCd | |
| | DP4 | 75 | SLLMWITQCFLPVF | 114 | 157-170 | peptide | Zeng, 2001 |
| | DP4 | 75 | LLEFYLAMPFATPM | 215 | 87-111 | peptide | Mandic, 2005 |
| | | | EAELARRSLAQ | | | | |
| | DR1 | 18 | LLEFYLAMPFATPM | 215 | 87-111 | peptide | Mandic, 2005 |
| | | | EAELARRSLAQ | | | | |
| | DR1 | 18 | EFYLAMPFATPM | 216 | 89-100 | protein | Chen, 2004 |
| | DR1 | 18 | PGVLLKEFTVSGNILT | 217 | 119-143 | peptide | Ayyoub, 2010 |
| | | | IRLTAADHR | | | | |
| | DR2 | 25 | RLLEFYLAMPFA | 218 | 86-97 | protein | Chen, 2004 |
| | DR3 | 21 | QGAMLAAQERRVP | 115 | ORF2 | protein | Slager, 2004a |
| | | | RAAEVPR | | (14-33) | | |
| | DR4 | 24 | PFATPMEAELARR | 219 | 95-107 | peptide | Mizote, 2010 |
| | DR4 | 24 | PGVLLKEFTVSGNILT | 220 | 119-138 | peptide and | Jager, 2000 |
| | | | IRLT | | | protein | Zarour, 2000 |
| | DR4 | 24 | VLLKEFTVSG | 221 | 121-130 | peptide | Zeng, 2000 |
| NY-ESO-1/ | DR4 | 24 | AADHRQLQLSISSCL | 116 | 139-156 | protein | Jager, 2000 |
| LAGE-2 | | | QQL | | | | |
| | DR4 | 24 | LLEFYLAMPFATPM | 215 | 87-111 | peptide | Mandic, 2005 |
| | | | EAELARRSLAQ | | | | |
| | DR52b | 25 | LKEFTVSGNILTIRL | 222 | 123-137 | protein | Bioley, 2009 |
| | DR7 | 25 | PGVLLKEFTVSGNILT | 217 | 119-143 | peptide | Zarour, 2002 |
| | | | IRLTAADHR | | | | |
| | DR7 | 25 | LLEFYLAMPFATPM | 215 | 87-111 | peptide | Mandic, 2005 |
| | | | EAELARRSLAQ | | | | |
| | DR8 | 4 | KEFTVSGNILT | 223 | 124-134 | peptide | Mizote, 2010 |
| | DR9 | 3 | LLEFYLAMPFATPM | 224 | 87-100 | peptide | Mizote, 2010 |
| | DR15 | 20 | AGATGGRGPRGAGA | 119 | 37-50 | protein | Hasegawa, 2006 |
| Neutrophil | | | VLQELNVTV | 225 | | | |
| granule | | | | | | | |
| proteases | | | | | | | |
| OFA-iLR | A2 | | ALCNTDSPL | 226 | | | Siegel, S. 2006 |
| | A2 | | LLAARAIVAI | 227 | | | Siegel, S. 2006 |
| PTH-rP | A2 | | TSTTSLELD | 228 | | | Francini, 2002 |
| | A2 | | FLHHLIAEIH | 229 | | | Francini, 2002 |
| S2 | A26 | | DLWKETVFT | 230 | | | Koga, 2003 |
| SAGE | A24 | 20 | LYATVIH DI | 231 | 715-723 | peptide | Miyahara, 2005 |
| Sp17 | A1 | 26 | ILDSSEEDK | 232 | 103-111 | protein | Chiriya-Internati, |
| | | | | | | | 2003 |
| SSX-2 | A2 | 44 | KASEKIFYV | 233 | 41-49 | autologous | Ayyoub, 2002 |
| | | | | | | tumor cells | |
| | A2 | | RLQGISPKI | 234 | | | Wagner, 2003 |
| | DP1 | 14 | EKIQKAFDDIAKYFSK | 235 | 19-34 | peptide | Ayyoub, 2004a |
| | DR1 | 18 | FGRLQGISPKI | 236 | 101-111 | peptide | Neumann, 2011 |
| | DR3 | 21 | WEKMKASEKIFYVY | 237 | 37-54 | peptide | Ayyoub, 2005a |
| | | | MKRK | | | | |
| | DR4 | 24 | KIFYVYMKRKYEAMT | 238 | 45-59 | peptide] | Neumann, 2004 |
| | DR11 | 25 | KIFYVYMKRKYEAM | 239 | 45-58 | protein | Ayyoub, 2004b |
| SSX-4 | DP10 | 2 | INKTSGPKRGKHAW | 240 | 151-170 | peptide | Ayyoub, 2005b |
| | | | THRLRE | | | | |
| SSX-4 | DR3 | 21 | YFSKKEWEKMKSSE | 241 | 31-50 | peptide | Ayyoub, 2005b |
| | | | KIVYVY | | | | |
| | DR8 | 4 | MKLNYEVMTKLGFK | 242 | 51-70 | peptide | Valmori, 2006 |
| | | | VTLPPF | | | | |
| | DR8 | 4 | KHAWTHRLRERKQL | 243 | 161-180 | peptide | Valmori, 2006 |
| | | | VVYEEI | | | | |
| | DR11 | 25 | LGFKVTLPPFMRSKR | 244 | 61-80 | peptide | Ayyoub, 2005b |
| | | | AADFH | | | | |
| | DR15 | 20 | KSSEKIVYVYMKLNY | 245 | 41-60 | peptide | Ayyoub, 2005b |
| | | | EVMTK | | | | |
| | DR52 | 41 | KHAWTHRLRERKQL | 243 | 161-180 | peptide | Valmori, 2006 |
| | | | VVYEEI | | | | |
| TAG | A3 | | RLSNRLLLR | 246 | | | Hogen, 2004 |
| TAG-1 | A2 | 44 | SLGWLFLLL | 247 | 78-86 | peptide | Adair, 2008 |
| | B8 | 14 | LSRLSNRLL | 248 | 42-50 | peptide | Adair, 2008 |
| TAG-2 | B8 | 14 | LSRLSNRLL | 248 | 42-50 | peptide | Adair, 2008 |
| TPBG | A1, A2, | | LTYVSFRNL | 249 | | | Shingler, 2009 |
| | A3, B7 | | | | | | |
| | A2 | | DLPAYVRNL | 250 | | | Smyth, 2006 |
| | A2 | | FLTGNQLAV | 251 | | | Shingler, 2009 |
| | A2 | | GAFEHLPSL | 252 | | | Smyth, 2006 |
| | A2 | | RLARLALVL | 253 | | | Shingler, 2009 |
| | Cw7 | | PLADLSPFA | 254 | | | Redchenko, 2006 |
| TRAG-3 | A2 | | ILLRDAGLV | 255 | | | Zhu, 2003 |
| | DR1 | 18 | CEFHACWPAFTVLGE | 256 | 34-48 | peptide | Janjic, 2006 |
| | DR4 | 24 | CEFHACWPAFTVLGE | 256 | 34-48 | peptide | Janjic, 2006 |
| | DR7 | 25 | CEFHACWPAFTVLGE | 256 | 34-48 | peptide | Janjic, 2006 |
| TRP2-6b | A2 | | ATTNILEHY | 257 | | | Khong, 2002 |
| TRP2-INT2g | A68 | 8 | EVISCKLIKR | 258 | intron 2 | autologous | Lupetti, 1998 |
| | | | | | | tumor cells | |
| TTK | A24 | | SYRNEIAYL | 259 | | | Suda T, 2007 |
| XAGE- | A2 | 44 | RQKKIRIQL | 260 | 21-29 | peptide | Ohue, 2012 |
| 1b/GAGED2a | DR4 | 24 | HLGSRQKKIRIQLRSQ | 261 | 17-32 | peptide | Ohue, 2012 |
| | DR9 | 3 | CATWKVICKSCISQT | 262 | 33-49 | autologous | Shimono, 2007 |
| | | | PG | | | tumor cells | |
| ART-4 | A24 | | AFLRHAAL | 263 | | | Kawano, 2000 |
| | A24 | | DYPSLSATDI | 264 | | | Kawano, 2000 |
| CDCA1/NUF2 | A2 | | YMMPVNSEV | 265 | | | Harao, 2008 |
| | A2 | | KLATAQFKI | 266 | | | Harao, 2008 |
| Cep55/c10orf3 | A24 | | VYVKGLLAKI | 267 | | | Inoda, 2009 |
| CML28 | A2 | | ALVDAGVPM | 268 | | | Han, 2006 |
| (EXOSC5) | | | | | | | |
| DAM-6, -10 | A2 | | FLWGPRAYA | 269 | | | Fleischhauer, 1998 |
| (MAGE-B1) | | | | | | | |
| IMP-3 | A2 | | RLLVPTQFV | 270 | | | Tomita, Y, 2010 |
| | A2 | | NLSSAEVVV | 271 | | | Tomita, Y, 2010 |
| | A24 | | KTVNELQNL | 272 | | | Suda T, 2007 |
| OVA66 | A2 | | FLPDHINIV | 273 | | | Jin, 2008 |
| OY-TES-1 | A24 | | KTPFVSPLL | 274 | | | Okumura,, 2005 |
| PASD1 | A2 | | QLLDGFMITL | 275 | | | Ait-Tahar, 2009 |
| | A2 | | YLVGNVCIL | 276 | | | Ait-Tahar, 2009 |
| | A2 | | ELSDSLGPV | 277 | | | Ait-Tahar, 2009 |
| RHAMM/CD168 | A2 | | ILSLELMKL | 278 | | | Greiner, 2005 |
| | A2 | | SLEENIVIL | 279 | | | Greiner, 2005 |
| SART-1 | A2 | | KGSGKMKTE | 280 | | | Shichijo, 1998 |
| | A24 | | EYRGFTQDF | 281 | | | Kikuchi, 1999 |
| SART-3 | A2 | | RLAEYQAYI | 282 | | | Ito, 2000 |
| | A2 | | LLQAEAPRL | 283 | | | Ito, 2000 |
| | A3 | | WLEYYNLER | 284 | | | Minami, 2007 |
| | A3 | | QIRPIFSNR | 285 | | | Minami, 2007 |
| | A24 | | VYDYNCHVDL | 286 | | | Yang, 1999 |
| | A24 | | AYIDFEMKI | 287 | | | Yang, 1999 |
| | A26 | | VYDYNCHVDL | 286 | | | Niu, 2009 |

DIFFERENTIATION

| | | | | | | | | |
|---|---|---|---|---|---|---|---|---|
| CEA | gut carcinoma | A2 | 44 | YLSGANLNLg | 288 | 605-613 | peptide | Tsang, 1995 |
| | | A2 | 44 | IMIGVLVGV | 289 | 691-699 | peptide | Kawashima, 1998a |
| | | A2 | 44 | GVLVGVALI | 290 | 694-702 | peptide | Alves, 2007 |
| | | A2 | | VLYGPDAPTV | 291 | | | Keogh, 2001 |
| | | A2 | | YLSGANLNV | 292 | | | Keogh, 2001 |
| | | A2 | | ATVGIMIGV | 293 | | | Keogh, 2001 |
| | | A3 | 22 | HLFGYSWYK | 294 | 61-69 | peptide | Kawashima, 1999 |
| | | A24 | 20 | QYSWFVNGTF | 295 | 268-277 | peptide | Nukaya, 1999 |
| | | A24 | 20 | TYACFVSNL | 296 | 652-660 | peptide | Nukaya, 1999 |
| | | B27 | | HRWCIPWQRL | 297 | | | Huarte, 2002 |
| | | DR3 | 21 | AYVCGIQNSVSANRS | 298 | 568-582 | peptide | Crosti, 2006 |
| | | DR4 | 24 | DTGFYTLHVIKSDLV | 299 | 116-140 | peptide | Shen, 2004 |
| | | | | NEEATGQFRV | | | | |
| | | DR4 | 24 | YSWRINGIPQQHTQV | 300 | 625-639 | peptide | Ruiz, 2004 |
| | | DR7 | 25 | TYYRPGVNLSLSC | 301 | 425-437 | peptide | Crosti, 2006 |
| | | DR7 | 25 | EIIYPNASLLIQN | 302 | 99-111 | peptide | Crosti, 2006 |
| | | DR9 | 3 | YACFVSNLATGRNNS | 303 | 653-667 | peptide | Kobayashi, 2002 |
| | | DR11 | 25 | LWWVNNQSLPVSP | 304 | 177-189 | peptide | Campi, 2003 |
| | | | | | | and | | |
| | | | | | | 355-367 | | |
| | | DR13 | 19 | LWWVNNQSLPVSP | 304 | 177-189 | peptide | Campi, 2003 |
| | | | | | | and | | |
| | | | | | | 355-367 | | |
| | | DR14 | 6 | LWWVNNQSLPVSP | 304 | 177-189 | peptide | Campi, 2003 |
| | | | | | | and | | |
| | | | | | | 355-367 | | |
| | | DR14 | 6 | EIIYPNASLLIQN | 302 | 99-111 | peptide | Crosti, 2006 |
| | | DR14 | 6 | NSIVKSITVSASG | 305 | 666-678 | peptide | Crosti, 2006 |
| gp100/ | melanoma | A2 | 44 | KTWGQYWQV | 306 | 154-162 | autologous | Bakker, 1995 |
| Pmel17 | | | | | | | tumor cells | Kawakami, 1995 |
| | | A2 | 44 | (A)MLGTHTMEV | 307 | 177(8)-186 | peptide | Tsai, 1997 |
| | | A2 | | IMDQVPFSV | 308 | | | Kawakami, 1995 |
| | | A2 | 44 | ITDQVPFSV | 309 | 209-217 | autologous | Kawakami, 1995 |
| | | | | | | | tumor cells | |
| | | A2 | 44 | YLEPGPVTA | 310 | 280-288 | autologous | Cox, 1994 |
| | | | | | | | tumor cells | |
| | | A2 | 44 | LLDGTATLRL | 311 | 457-466 | autologous | Kawakami, 1994a |
| | | | | | | | tumor cells | |
| | | A2 | 44 | VLYRYGSFSV | 312 | 476-485 | autologous | Kawakami, 1995 |
| | | | | | | | tumor cells | |
| | | A2 | 44 | SLADTNSLAV | 313 | 570-579 | peptide | Tsai, 1997 |
| gp100/ | melanoma | A2 | 44 | RLMKQDFSV | 314 | 619-627 | autologous | Kawakami, 1998 |
| Pmel17 | | | | | | | tumor cells | |
| | | A2 | 44 | RLPRIFCSC | 315 | 639-647 | autologous | Kawakami, 1998 |
| | | | | | | | tumor cells | |
| | | A3 | 22 | LIYRRRLMK | 316 | 614-622 | autologous | Kawakami, 1998 |
| | | | | | | | tumor cells | |
| | | A3 | 22 | ALLAVGATK | 317 | 17-25 | autologous | Skipper, 1996a |
| | | | | | | | tumor cells | |
| | | A3 | 22 | IALNFPGSQK | 318 | 86-95 | peptide | Kawashima, 1998b |
| | | A3 | 22 | RSYVPLAHR | 319 | 195-202 and | autologous | Michaux, 2014 |
| | | | | | | 191 or 192e | tumor cells | |
| | | A3 | 22 | ALNFPGSQK | 320 | 87-95 | peptide | Kawashima, 1998b |
| | | A11 | 13 | ALNFPGSQK | 320 | 87-95 | peptide | Kawashima, 1998b |
| | | A24 | 20 | VYFFLPDHL | 321 | intron 4 | autologous | Robbins, 1997 |
| | | | | | | | tumor cells | |
| | | A32 | 8 | RTKQLYPEW | 322 | 40-42 | autologous | Vigneron, 2004 |
| | | | | | | and | tumor cells | |
| | | | | | | 47-52e | | |
| | | A68 | 8 | HTMEVTVYHR | 323 | 182-191 | autologous | Sensi, 2002 |
| | | | | | | | tumor cells | |
| | | B7 | 17 | SSPGCQPPA | 324 | 529-537 | autologous | ennerz, 2005 |
| | | | | | | | tumor cells | |
| | | B35 | 20 | VPLDCVLYRY | 325 | 471-480 | autologous | Benlalam, 2003 |
| | | | | | | | tumor cells | |
| | | B35 | 20 | LPHSSSHWL | 326 | 630-638 | autologous | Vigneron, 2005 |
| | | | | | | | tumor cells | |
| | | Cw8 | -c | SNDGPTLI | 327 | 71-78 | autologous | Castelli, 1999 |
| | | | | | | | tumor cells | |
| | | DQ6 | 63 | GRAMLGTHTMEVT | 328 | 175-189 | peptide | Kobayashi, 2001 |
| | | | | VY | | | | |
| | | DR4 | 24 | WNRQLYPEWTEAQ | 329 | 44-59 | peptide | Touloukian, 2000 |
| | | | | RLD | | | | |
| | | DR7 | 25 | TTEWVETTARELPIP | 330 | 420-437 | protein | Parkhurst, 2004 |
| | | | | EPE | | | | |
| | | DR7 | 25 | TGRAMLGTHTMEV | 331 | 174-190 | retrovirus- | Lapointe, 2001 |
| | | | | TVYH | | | dendritic cells | |
| | | DR53 | 49 | GRAMLGTHTMEVT | 328 | 175-189 | peptide | Kobayashi, 2001 |
| | | | | VY | | | | |
| mammaglobin-A | breast cancer | A2 | | FLNQTDETL | 332 | | | Jaramillo, 2004 |
| | | A2 | | MQLIYDSSL | 333 | | | Jaramillo, 2004 |
| | | A2 | | KLLMVLMLA | 334 | | | Jaramillo, 2004 |
| | | A2 | | LIYDSSLCDL | 335 | | | Jaramillo, 2004 |
| | | A3 | | AIDELKECF | 336 | | | Jaramillo, 2002 |
| | | A3 | | TTNAIDELK | 337 | | | Jaramillo, 2002 |
| | | A3 | 22 | PLLENVISK | 338 | 23-31 | peptide | Jaramillo, 2002 |
| | | A3 | | KLLMVLMLA | 334 | | | Jaramillo, 2002 |
| Melan-A/ | melanoma | A2 | 44 | (E)AAGIGILTV | 339 | 26(27)-35 | autologous | Kawakami, 1994b |
| MART-1 | | | | | | | tumor cells | |
| | | A2 | 44 | ILTVILGVL | 340 | 32-40 | autologous | Castelli, 1995 |
| | | | | | | | tumor cells | |
| | | B35 | 20 | EAAGIGILTV | 341 | 26-35 | autologous | Benlalam, 2003 |
| | | | | | | | tumor cells | |
| | | B45 | 2 | AEEAAGIGIL(T) | 342 | 24-33(34) | autologous | Schneider, 1998 |
| | | | | | | | tumor cells | |
| | | Cw7 | 41 | RNGYRALMDKS | 343 | 51-61 | peptide | Larrieu, 2008 |
| | | DP5 | 3 | YTTAEEAAGIGILTVI | 344 | 21-50 | peptide | Meng, 2011 |
| | | | | LGVLLLIGCWYCRR | | | | |
| | | DQ6 | 63 | EEAAGIGILTVI | 345 | 25-36 | peptide | Bioley, 2006 |
| | | DR1 | 18 | AAGIGILTVILGVL | 346 | 27-40 | peptide | Bioley, 2006 |
| | | DR1 | 18 | APPAYEKLpSAEQf | 347 | 100-111 | peptide | Depontieu, 2009 |
| | | DR3 | 21 | EEAAGIGILTVI | 345 | 25-36 | peptide | Bioley, 2006 |
| | | DR4 | 24 | RNGYRALMDKSLHV | 348 | 51-73 | peptide | Zarour, 2000 |
| | | | | GTQCALTRR | | | | |
| | | DR11 | 25 | MPREDAHFIYGYPK | 349 | 20-Jan | peptide | Godefroy, 2006 |
| | | | | KGHGHS | | | | |
| | | DR52 | 41 | KNCEPVVPNAPPAY | 350 | 91-110 | peptide | Godefroy, 2006 |
| | | | | EKLSAE | | | | |
| MC1R | | A2 | | TILLGIFFL | 351 | | | Salazar-Onfray, |
| | | | | | | | | 1997 |
| | | A3 | | FLALIICNA | 352 | | | Salazar-Onfray, |
| | | | | | | | | 1997 |
| Mesothelin | | A2 | | KLLGPHVEGL | 353 | | | Yokokawa, 2005 |
| | | A2 | | KLLGPHVLGV | 354 | | | Yokokawa, 2005 |
| NGEP | | A2 | | GLFDEYLEMV | 355 | | | Cereda, 2010 |
| NY-BR-1 | breast cancer | A2 | 44 | SLSKILDTV | 356 | 904-912 | peptide | Wang, 2006 |
| | | A2 | | LLSHGAVIEV | 357 | | | Jager, 2005 |
| OA1 | melanoma | A24 | 20 | LYSACFWWL | 358 | 126-134 | peptide | Touloukian, 2003 |
| PAP | prostate | A2 | 44 | FLFLLFFWL | 9 | 18-26 | peptide | Olson, 2010 |
| | cancer | A2 | 44 | TLMSAMTNL | 10 | 112-120 | peptide | Olson, 2010 |
| | | A2 | 44 | ALDVYNGLL | 8 | 299-307 | peptide | Olson, 2010 |
| P Polypeptide | | A2 | | IMLCLIAAV | 359 | | | Touloukian, 2001 |
| PSA | prostate | A2 | 44 | FLTPKKLQCV | 360 | 165-174 | peptide | Correale, 1997 |
| | carcinoma | A2 | 44 | VISNDVCAQV | 361 | 178-187 | peptide | Correale, 1997 |
| RAB38/NY- | melanoma | A2 | 44 | VLHWDPETV | 362 | 50-58 | peptide | Walton, 2006 |
| MEL-1 | | | | | | | | |
| TARP | | A2 | | FLRNFSLMV | 363 | | | Oh, 2004 |
| | | A2 | | FVFLRNFSL | 364 | | | Oh, 2004 |
| | | A2 | | FLRNFSLML | 365 | | | Oh, 2004 |
| TRP-1/gp75 | melanoma | A31 | 5 | MSLQRQFLR | 366 | alt. ORF | autologous | Wang, 1996a |
| | | | | | | | tumor cells | |
| | | DR4 | 24 | ISPNSVFSQWRVVC | 367 | 277-297 | peptide | Touloukian, 2002 |
| | | | | DSLEDYD | | | | |
| | | DR15 | 20 | SLPYWNFATG | 368 | 245-254 | autologous | Robbins, 2002 |
| | | | | | | | tumor cells | |
| | | DR17 | 21 | SQWRVVCDSLEDYDT | 369 | 284-298 | peptide | Osen, 2010 |
| TRP-2 | melanoma | A1, A2 | | VYDFFVWLHY | 370 | | | Paschen, 2005 |
| | | A2 | | SLDDYNHLV | 371 | | | Sun, 2000 |
| | | A2 | | FVWLHYYSV | 372 | | | Bredenbeck. 2005 |
| | | A2 | 44 | SVYDFFVWL | 373 | 180-188 | peptide | Parkhurst, 1998 |
| | | A2 | 44 | TLDSQVMSL | 374 | 360-368 | peptide | Noppen, 2000 |
| | | A31 | 5 | LLGPGRPYR | 375 | 197-205 | autologous | Wang, 1996b |
| | | | | | | | tumor cells | Wang, 1998 |
| | | A33 | 5 | LLGPGRPYR | 375 | 197-205 | autologous | Wang, 1998 |
| | | | | | | | tumor cells | |
| | | Cw8 | -c | ANDPIFVVL | 376 | 387-395 | autologous | Castelli, 1999 |
| | | | | | | | tumor cells | |
| | | DR3 | 21 | QCTEVRADTRPWSGP | 377 | 60-74 | peptide | Paschen, 2005 |
| | | DR15 | 20 | ALPYWNFATG | 378 | 241-250 | autologous | Robbins, 2002 |
| | | | | | | | tumor cells | |
| tyrosinase | melanoma | A1 | 26 | KCDICTDEY | 379 | 243-251 | autologous | Kittlesen, 1998 |
| | | | | | | | tumor cells | |
| | | A1 | | DSDPDSFQDY | 380 | | | Schreibenbogen, |
| | | | | | | | | 2002 |
| | | A1 | 26 | SSDYVIPIGTY | 381 | 146-156 | autologous | Kawakami, 1998 |
| | | | | | | | tumor cells | |
| tyrosinase | melanoma | A2 | 44 | MLLAVLYCL | 382 | 1-9 | autologous | Wolfel, 1994 |
| | | | | | | | tumor cells | |
| | | A2 | 44 | CLLWSFQTSA | 383 | 8-17 | peptide | Riley, 2001 |
| | | A2 | 44 | YMDGTMSQV | 384 | 369-377 | autologous | Wolfel, 1994 |
| | | | | | | | tumor cells | Skipper, 1996b |
| | | A24 | 20 | AFLPWHRLF | 385 | 206-214 | autologous | Kang, 1995 |
| | | | | | | | tumor cells | |
| | | A24 | 20 | IYMDGTADFSF | 386 | 368-373 and | autologous | Dalet, 2011 |
| | | | | | | 336-340e | tumor cells | |
| | | A26 | 8 | QCSGNFMGF | 387 | 90-98 | autologous | Lennerz, 2005 |
| | | | | | | | tumor cells | |
| | | B35 | 20 | TPRLPSSADVEF | 388 | 309-320 | autologous | Benlalam, 2003 |
| | | | | | | | tumor cells | |
| | | B35 | 20 | LPSSADVEF | 389 | 312-320 | autologous | Morel, 1999 |
| | | | | | | | tumor cells | |
| | | B38 | 5 | LHHAFVDSIF | 390 | 388-397 | autologous | Lennerz, 2005 |
| | | | | | | | tumor cells | |
| | | B44 | 21 | SEIWRDIDFd | 391 | 192-200 | autologous | Brichard, 1996 |
| | | | | | | | tumor cells | |
| | | DR4 | 24 | QNILLSNAPLGPQFP | 392 | 56-70 | autologous | Topalian, 1996 |
| | | | | | | | tumor cells | |
| | | DR4 | 24 | SYLQDSDPDSFQD | 393 | 450-462 | autologous | Topalian, 1996 |
| | | | | | | | tumor cells | |
| | | DR15 | 20 | FLLHHAFVDSIFEQW | 394 | 386-406 | autologous | Kobayashi, 1998 |
| | | | | LQRHRP | | | tumor cells | |
| human | Differentiation | | | | | | | |
| chorionic | | | | | | | | |
| gonadotropin | | | | | | | | |
| P501s | Differentiation | Cw5 | | SACDVSVRVV | 395 | | Peptide | Friedman, 2004 |
| (prostein) | | Cw5 | | YTDFVGEGL | 396 | | peptide | Friedman, 2004 |

OVEREXPRESSED

| | | | | | | | | |
|---|---|---|---|---|---|---|---|---|
| ADAM17 | | A2 | | YLIELIDRV | 397 | | | Sinnathamby G, |
| | | | | | | | | 2011 |
| adipophilin | adipocytes, | A2 | 44 | SVASTITGV | 398 | 129-137 | peptide | Schmidt, 2004 |
| | macrophages | | | | | | | |
| ADP- | | A2 | | CITFQVWDV | 399 | | | Nonaka, 2002 |
| ribosylation | | A2 | | FLPHFQALHV | 400 | | | Nonaka, 2002 |
| factor | | | | | | | | |
| AIM-2 | ubiquitous | A1 | 26 | RSDSGQQARY | 401 | intron | autologous | Harada, 2001 |
| | (low level) | | | | | | tumor cells | |
| ALDH1A1 | mucosa, | A2 | 44 | LLYKLADLI | 402 | 88-96 | peptide | Visus, 2007 |
| | keratinocytes | | | | | | | |
| ALK | | A2 | | SLAMLDLLHV | 403 | | | Passoni, 2002 |
| ATIC (AICRT) | | A2 | | MVYDLYKTL | 404 | | | Shichijo, 2004 |
| | | A2 | | RLDFNLIRV | 405 | | | Shichijo, 2004 |
| BA46 (MFGE8) | | A2 | | GLQHWVPEL | 406 | | | Carmon, 2002 |
| | | A2 | | NLFETPVEA | 407 | | | Carmon, 2002 |
| BAP31 | | A2 | | KLDVGNAEV | 408 | | | Ramakrishna, 2003 |
| BAX-delta | | A2 | | YLLQGMIAAV | 409 | | | Maia, 2005 |
| | | A2 | | YLQGMIAAV | 410 | | | Maia, 2005 |
| Bcl-2 | | A2 | | PLFDFSWLSL | 411 | | | Andersen, 2005 |
| | | A2 | | WLSLKTLLSL | 412 | | | Andersen, 2005 |
| BCLX (L) | ubiquitous | A2 | 44 | YLNDHLEPWI | 413 | 173-182 | peptide | Sorensen, 2007 |
| | (low level) | | | | | | | |
| BING-4 | ubiquitous | A2 | 44 | CQWGRLWQL | 414 | ORF2 | anti-CD3 | Rosenberg, 2002 |
| | (low level) | | | | | | | |
| BTBD2 | | A24 | | VFLPCDSWNL | 415 | | | Yamada, 2003 |
| C19orf48 | | A2 | | CIPPDSLLFPA | 416 | | | Tykodi. S 2008 |
| CA125 | | A2 | | YTLDRDSLYV | 417 | | | Bellone, 2009 |
| Cadherin 3/P- | | A2 | | FIIENLKAA | 418 | | | Imai, 2008 |
| cadherin | | A2 | | FILPVLGAV | 419 | | | Imai, 2008 |
| CALCA | thyroid | A2 | 44 | VLLQAGSLHA | 420 | 16-25 | autologous | El Hage, 2008 |
| | | | | | | | tumor cells | |
| CLCA2 | | A2 | | LLGNCLPTV | 421 | | | Konopitzky, 2002 |
| | | A2 | | SLQALKVTV | 422 | | | Konopitzky, 2002 |
| CLP | | A2 | NLVRDD | | | | | Nakatsura, 2002 |
| | | | GSAV | | | | | |
| | | | (SEQ ID | | | | | |
| | | | NO: 13) | | | | | |
| | | A2 | RLFAFV | | | | | Nakatsura, 2002 |
| | | | RFT | | | | | |
| | | | (SEQ ID | | | | | |
| | | | NO: 14) | | | | | |
| | | A2 | VVQNFA | | | | | Nakatsura, 2002 |
| | | | KE FV | | | | | |
| | | | (SEQ ID | | | | | |
| | | | NO: 15) | | | | | |
| CD45 | proliferating | A24 | 20 | KFLDALISL | 423 | 556-564 | peptide | Tomita, 2011a |
| | cells, testis, | | | | | | | |
| | multiple | | | | | | | |
| | tissues (low | | | | | | | |
| | level) | | | | | | | |
| CD274 | multiple | A2 | 44 | LLNAFTVTV | 424 | 15-23 | peptide | Munir, 2012 |
| | tissues (lung, | | | | | | | |
| | heart, . . . ) | | | | | | | |
| | and | | | | | | | |
| | induced by | | | | | | | |
| | IFN-γ | | | | | | | |
| CDKN1A | | A2 | | FAWERVRGL | 425 | | | Li, 2006 |
| | | A2 | | GLGLPKLYL | 426 | | | Li, 2006 |
| | | A2 | | LMAGCIQEA | 427 | | | Li, 2006 |
| Cdr2 | | A2 | | LLEEMFLTV | 428 | | | Santomasso, 2007 |
| CPSF | ubiquitous | A2 | 44 | KVHPVIWSL | 429 | 250-258 | autologous | Maeda, 2002 |
| | (low level) | | | | | | tumor cells | |
| | | A2 | 44 | LMLQNALTTM | 430 | 1360-1369 | autologous | Maeda, 2002 |
| | | | | | | | tumor cells | |
| c-MET | | A2 | | YVDPVITSI | 431 | | | Schag, 2004 |
| COA-1 | | A2 | | FMTRKLWDL | 432 | | | Maccalli, 2008 |
| (UBXN11) | | A2 | | RLLASLQDL | 433 | | | Maccalli, 2008 |
| Cox2 | | A2 | | ALYGDIDAV | 434 | | | Gao, Y, 2009 |
| | | A3 | | ALYGDIDAV | 434 | | | Gao, Y, 2009 |
| Cyclin I | | A2 | | LLDRFLATV | 435 | | | Ramakrishna, 2003 |
| cyclin B1 | | A2 | | ILIDWLVQV | 436 | | | Andersen, 2011 |
| | | A2 | | AKYLMELTM | 437 | | | Kao, 2001 |
| | | A2 | | AGYLMELCC | 438 | | | Kao, 2001 |
| cyclin D1 | ubiquitous | A2 | 44 | LLGATCMFV | 439 | 101-109 | peptide | Kondo, 2008 |
| | (low level) | DR4 | 24 | NPPSMVAAGSVVA | 440 | 198-212 | peptide | Dengjel, 2004 |
| | | | | AV | | | | |
| cyclophilin B | | A2 | | VLEGMEVV | 441 | | | Tamura, 2001 |
| (Cyp-B) | | A2 | | KLKHYGPGWV | 442 | | | Tamura, 2001 |
| | | A24 | | DFMIQGGDF | 443 | | | Gomi, 1999 |
| | | A24 | | KFHRVIKDF | 444 | | | Gomi, 1999 |
| CYP1B1 | | A2 | | WLQYFPNPV | 445 | | | Maecker, 2005 |
| DKK1 | testis, | A2 | 44 | ALGGHPLLGV | 446 | 20-29 | peptide | Qian, 2007 |
| | prostate, | | | | | | | |
| | mesenchymal | | | | | | | |
| | stem cells | | | | | | | |
| EGFR | | A2 | | ITDFGLAKL | 447 | | | Filho, 2009 |
| | | A2 | | ITDFGLAKL | 447 | | | Filho, 2009 |
| ENAH (hMena) | breast, | A2 | 44 | TMNGSKSPV | 448 | 502-510 | peptide | Di Modugno, 2004 |
| | prostate | | | | | | | |
| | stroma and | | | | | | | |
| | epithelium of | | | | | | | |
| | colon-rectum, | | | | | | | |
| | pancreas, | | | | | | | |
| | endometrium | | | | | | | |
| EpCAM | epithelial cells | A2 | | YQLDPKFIV | 449 | | | Trojan, 2001 |
| | | A2 | | GLKAGVIAV | 450 | | | Nagorsen, 2000 |
| | | A2 | | ILYENNVITV | 451 | | | Trojan, 2001 |
| | | A2 | | ILYENNVIV | 452 | | | Trojan, 2001 |
| | | A24 | 20 | RYQLDPKFI | 453 | 173-181 | peptide | Tajima, 2004 |
| EphA2 | | A2 | | VLLLVLAGV | 454 | | | Tatsumi, 2003 |
| | | A2 | | TLADFDPRV | 455 | | | Tatsumi, 2003 |
| | | A2 | | VLAGVGFFI | 456 | | | Alves, 2003; Easty |
| | | | | | | | | 1995 |
| | | A2 | | IMNDMPIYM | 457 | | | Alves, 2003; Easty |
| | | | | | | | | 1995 |
| EphA3 | many | DR11 | 25 | DVTFNIICKKCG | 458 | 356-367 | autologous | Chiari, 2000 |
| | | | | | | | tumor cells | |
| EZH2 | ubiquitous | A2 | 44 | FMVEDETVL | 459 | 120-128 | peptide | Itoh, 2007 |
| | (low level) | A2 | 44 | FINDEIFVEL | 460 | 165-174 | peptide | Itoh, 2007 |
| | | A24 | 20 | KYDCFLHPF | 461 | 291-299 | peptide | Ogata, 2004 |
| | | A24 | 20 | KYVGIEREM | 462 | 735-743 | peptide | Ogata, 2004 |
| FGF5 | brain, kidney | A3 | 22 | NTYASPRFKf | 463 | 172-176 | autologous | Hanada, 2004 |
| | | | | | | and | tumor cells | |
| | | | | | | 217-220 | | |
| glypican-3 | placental and | A2 | 44 | FVGEFFTDV | 464 | 144-152 | peptide | Komori, 2006 |
| | multiple | A24 | 20 | EYILSLEEL | 465 | 298-306 | peptide | Komori, 2006 |
| | tissues | | | | | | | |
| G250/MN/ | stomach, liver, | A2 | 44 | HLSTAFARV | 466 | 254-262 | peptide | Vissers, 1999 |
| CAIX | pancreas | | | | | | | |
| HBD | | A24 | | KYLKLSSSEL | 467 | | | Yamada, 2003 |
| hCG-beta | | A2 | | TMTRVLQGV | 468 | | | Dangles, 2002 |
| | | A2 | | GVNPVVSYAV | 469 | | | Dangles, 2002 |
| | | A2 | | VLQVGLPAL | 470 | | | Dangles, 2002 |
| Heparanase | | A2 | | PAFSYSFFV | 471 | | | Chen T, 2008 |
| | | A2 | | LLLGPLGPL | 472 | | | Sommerfeldt, 2006 |
| | | A2 | | KMLKSFLKA | 473 | | | Chen T, 2008 |
| | | A2 | | ALPPPLMLL | 474 | | | Sommerfeldt, 2006 |
| | | A2 | | WLSLLFKKL | 475 | | | Chen T, 2008 |
| HER2/neu | ubiquitous | A2 | 44 | KIFGSLAFL | 476 | 369-377 | autologous | Fisk, 1995 |
| | (low level) | | | | | | tumor cells | |
| | | A2 | 44 | IISAVVGIL | 477 | 654-662 | peptide | Brossart, 1998 |
| | | A2 | 44 | ALCRWGLLL | 478 | 13-May | peptide | Kawashima, 1998 |
| | | A2 | 44 | ILHNGAYSL | 479 | 435-443 | peptide | Kawashima, 1998 |
| | | A2 | 44 | RLLQETELV | 480 | 689-697 | peptide | Rongcun, 1999 |
| | | A2 | 44 | VVLGVVFGI | 481 | 665-673 | peptide | Rongcun, 1999 |
| | | A2 | 44 | YMIMVKCWMI | 482 | 952-961 | peptide | Rongcun, 1999 |
| | | A2 | 44 | HLYQGCQVV | 483 | 48-56 | peptide | Scardino, 2001 |
| | | A2 | 44 | YLVPQQGFFC | 484 | 1023-1032 | peptide | Scardino, 2001 |
| | | A2 | 44 | PLQPEQLQV | 485 | 391-399 | peptide | Scardino, 2002 |
| | | A2 | 44 | TLEEITGYL | 486 | 402-410 | peptide | Scardino, 2002 |
| | | A2 | 44 | ALIHHNTHL | 487 | 466-474 | peptide | Scardino, 2002 |
| | | A2 | 44 | PLTSIISAV | 488 | 650-658 | peptide | Scardino, 2002 |
| | | A2 | | KLFGSLAFV | 489 | | | Keogh, 2001 |
| | | A2 | | ITDFGLARL | 490 | | | Filho, 2009 |
| | | A2 | | KVFGSLAFV | 491 | | | Keogh, 2001 |
| | | A2 | | AVVGILLVV | 492 | | | Gritzapis, 2009 |
| | | A2 | | QLFEDNYAL | 493 | | | Kono, 1998 |
| | | A2 | | QIAKGMSYL | 494 | | | Lekka, 2009 |
| | | A2 | | LIAHNQVRQV | 495 | | | Gritzapis, 2008 |
| | | A3 | 22 | VLRENTSPK | 496 | 754-762 | peptide | Kawashima, 1999 |
| | | A24 | 20 | TYLPTNASL | 497 | 63-71 | peptide | Okugawa, 2000 |
| HIFPH3 | | A24 | | RYAMTVWYF | 498 | | | Sato, 2008 |
| HLA-DOB | B | A2 | 44 | FLLGLIFLL | 499 | 232-240 | peptide | Kang, 2013 |
| | lymphocytes, | | | | | | | |
| | monocytes, | | | | | | | |
| | blood cells, | | | | | | | |
| | adrenals, . . . | | | | | | | |
| HM1.24 | | A2 | | LLLGIGILV | 500 | | | Hundemer, 2006 |
| HMW-MAA | | A2 | | LLQLGYSGRL | 501 | | | Murray, 2004 |
| | | A2 | | LLQLYSGRL | 502 | | | Murray, 2004 |
| Hepsin | kidney, liver, | A2 | 44 | SLLSGDWVL | 503 | 191-199 | peptide | Guo, 2013 |
| | skin, . . . | A2 | 44 | GLQLGVQAV | 504 | 229-237 | peptide | Guo, 2013 |
| | | A2 | 44 | PLTEYIQPV | 505 | 268-276 | peptide | Guo, 2013 |
| HO-1 | | B8 | | APLLRWVL | 506 | | | Flad, 2006 |
| Hsp70 | | A2 | | LLLLDVAPL | 507 | | | Faure, 2004 |
| | | A2 | | LLDVAPLSL | 508 | | | Faure, 2004 |
| HST-2 (FGF-6) | | A31 | | YSWMDISCWI | 509 | | | Suzuki, 1999 |
| ICE | | B7 | | SPRWWPTCL | 510 | | | Ronsin C. 1999 |
| IDO1 | lymph nodes, | A2 | 44 | ALLEIASCL | 511 | 199-207 | peptide | Sorensen, 2009 |
| | placenta, and | | | | | | | |
| | many cell | | | | | | | |
| | types in the | | | | | | | |
| | course of | | | | | | | |
| | inflammatory | | | | | | | |
| | response | | | | | | | |
| IEX-1 | | A11 | | APAGRPSASR | 512 | | | Matsueda, 2007 |
| | | A11 | | RSRRVLYPR | 513 | | | Matsueda, 2007 |
| | | A31 | | RSRRVLYPR | 513 | | | Matsueda, 2007 |
| | | A31 | | APAGRPSASR | 512 | | | Matsueda, 2007 |
| | | A33 | | RSRRVLYPR | 513 | | | Sasada, 2004 |
| | | A33 | | APAGRPSAS | 514 | | | Sasada, 2004 |
| | | A33 | | vlyprvvrr | 515 | | | Sasada, 2004 |
| IGF2B3 | ubiquitous | A2 | 44 | NLSSAEVVV | 271 | 515-523 | peptide | Tomita, 2011b |
| | (low level) | A3 | 44 | RLLVPTQFV | 270 | 199-207 | peptide | Tomita, 2011b |
| IL13Ralpha2 | | A2 | 44 | WLPFGFILI | 516 | 345-353 | peptide | Okano, 2002 |
| | | A24 | | WYEGLDHAL | 517 | | | Shimato, 2008 |
| integrin beta | | A2 | | ALMEQQHYV | 518 | | | Ramakrishna, 2003 |
| subunit | | | | | | | | |
| Intestinal | liver, | B7 | 17 | SPRWWPTCL | 510 | alt. ORF | autologous, | Ronsin, 1999 |
| carboxyl | intestine | | | | | | tumor cells | |
| esterase | kidney | | | | | | | |
| alpha- | liver | A2 | 44 | GVALQTMKQ | 519 | 542-550 | adenovirus- | Butterfield, 1999 |
| foetoprotein | | | | | | | dendritic cells | |
| | | A2 | 44 | FMNKFIYEI | 520 | 158-166 | peptide | Pichard, 2008 |
| | | A2 | | GLSPNLNRFL | 521 | | | Butterfield, 2001 |
| | | A2 | | PLFQVPEPV | 522 | | | Butterfield, 2001 |
| | | A3 | | ILLWAARYD | 523 | | | Liu Y, 2006 |
| | | A24 | | EYSRRHPQL | 524 | | | Mizukoshi, 2006 |
| | | A24 | | AYTKKAPQL | 525 | | | Mizukoshi, 2006 |
| | | A24 | | EYYLQNAFL | 526 | | | Mizukoshi, 2006 |
| | | A24 | | KYIQESQAL | 527 | | | Mizukoshi, 2006 |
| | | A24 | | RSCGLFQKL | 528 | | | Mizukoshi, 2006 |
| | | DR13 | 19 | QLAVSVILRV | 529 | 364-373 | peptide | Alisa, 2005 |
| JARID1B | | A2 | | QLYALPCVL | 530 | | | Coleman, JA, 2010 |
| Keratin 18 | | A2 | | ALLNIKVKL | 531 | | | Weinschenk, 2002 |
| Kallikrein 4 | prostate and | A2 | 44 | FLGYLILGV | 532 | 19-Nov | peptide | Wilkinson, 2012 |
| | ovarian cancer | DP4 | 75 | SVSESDTIRSISIAS | 533 | 125-139 | peptide | Hural, 2002 |
| | | DR4 | 24 | LLANGRMPTVLQCVN | 534 | 155-169 | peptide | Hural, 2002 |
| | | DR7 | 25 | RMPTVLQCVNVSVVS | 535 | 160-174 | peptide | Hural, 2002 |
| KIF20A | ubiquitous | A2 | 44 | LLSDDDVVV | 536 | 20-Dec | peptide | Imai, 2011 |
| | (low level) | A2 | 44 | AQPDTAPLPV | 537 | 284-293 | peptide | Imai, 2011 |
| | | A2 | 44 | CIAEQYHTV | 538 | 809-817 | peptide | Imai, 2011 |
| L10a | | A26 | | ETVELQISL | 539 | | | Koga, 2003 |
| | | A26 | | TLYEAVREV | 540 | | | Koga, 2003 |
| Lck | | A2 | | KLVERLGAA | 541 | | | Imai, 2001 |
| | | A2 | | DVWSFGILL | 542 | | | Imai, 2001 |
| | | A24 | | TFDYLRSVL | 543 | | | Harashima, 2001 |
| | | A24 | | HYTNASDGL | 544 | | | Harashima, 2001 |
| | | A24 | | DYLRSVLEDF | 545 | | | Harashima, 2001 |
| Livin (ML-IAP) | | A2 | | QLCPICRAPV | 546 | | | Andersen, 2004 |
| | | A2 | | RLASFYDWLP | 547 | | | Schmollinger, 2003 |
| | | A2 | | SLGSPVLGL | 548 | | | Schmollinger, 2003 |
| LRRC8A | | B7 | | GPRESRPPA | 549 | | | Baba, T; 2010 |
| Lengsin | eye lens and | A2 | 44 | FLPEFGISSA | 550 | 270-279 | peptide | Nakatsugawa, 2011 |
| | low level in | | | | | | | |
| | multiple | | | | | | | |
| | tissues | | | | | | | |
| M2BP | | A2 | | RIDITLSSV | 551 | | | Ozaki, 2004 |
| | | A24 | | GYCASLFAIL | 552 | | | Kontani, 2004 |
| M-CSF | liver, kidney | B35 | 20 | LPAVVGLSPGEQEY | 553 | alt. ORF | autologous | Probst-Kepper, |
| | | | | | | | tumor cells | 2001 |
| MCSP | endothelial | DR11 | 25 | VGQDVSVLFRVTGA | 554 | 693-708 | peptide | Erfurt, 2007 |
| | cells, | | | LQ | | | | |
| | chondrocytes, | | | | | | | |
| | smooth | | | | | | | |
| | muscle cells | | | | | | | |
| mdm-2 | ubiquitous | A2 | 44 | VLFYLGQY | 555 | 53-60 | tumor lysate- | Asai, 2002 |
| | (brain, | | | | | | pulsed APCs | |
| | muscle, lung) | A2 | | LLGDLFGV | 556 | | | Mayr, 2006 |
| Meloe | ubiquitous | A2 | 44 | TLNDECWPA | 557 | 36-44 | tumor- | Godet, 2008 |
| | (low level) | | | | | | infiltrating | |
| | | | | | | | lymphocytes | |
| | | DQ2 | 41 | ERISSTLNDECWPA | 558 | 31-44 | peptide/protein | Bobinet, 2012 |
| | | DQ6 | 63 | FGRLQGISPKI | 236 | 32-44 | peptide | Rogel, 2011 |
| | | DR1 | 18 | TSREQFLPSEGAA | 559 | 23-Nov | peptide/protein | Bobinet, 2012 |
| | | DR11 | 25 | CPPWHPSERISSTL | 560 | 24-37 | peptide | Rogel, 2011 |
| Meloe-2 | | A2 | | RLPPKPPLA | 561 | | | Godet, 2010 |
| | | A2 | | RCPPKPPLA | 562 | | | Godet, 2010 |
| MG50 | | A2 | | TLKCDCEIL | 563 | | | Mitchell, 2000 |
| | | A2 | | RLGPTLMCL | 564 | | | Mitchell, 2000 |
| | | A2 | | LLLEAVPAV | 565 | | | Mitchell, 2000 |
| | | A2 | | WLPKILGEV | 566 | | | Mitchell, 2000 |
| | | A2 | | VLSVNVPDV | 567 | | | Mitchell, 2000 |
| | | A2 | | CMHLLLEAV | 568 | | | Mitchell, 2000 |
| Midkine | ubiquitous | A2 | 44 | ALLALTSAV | 569 | 13-21 | peptide | Kerzerho, 2010 |
| | (low level) | | | | | | | |
| | | A2 | 44 | AQCQETIRV | 570 | 114-122 | peptide | Kerzerho, 2010 |
| | | DR4 | 24 | LTLLALLALTSAVAK | 571 | 23-Sep | peptide | Kerzerho, 2013 |
| MMP-2 | ubiquitous | A2 | 44 | GLPPDVQRVh | 572 | 560-568 | autologous | Godefroy, 2005 |
| | | | | | | | tumor cells | |
| MMP-7 | ubiquitous | A3 | 22 | SLFPNSPKWTSK | 573 | 96-107 | peptide | Yokoyama, 2008 |
| | (low level) | | | | | | | |
| MPP-11 | | A2 | | QLLIKAVNL | 574 | | | Siegel S, 2010 |
| | | A2 | | STLCQVEPV | 575 | | | AI Qudaihi, 2010 |
| MRP3 | | A24 | | AYVPQQAWI | 576 | | | Yamada, 2001 |
| | | A24 | | VYSDADIFL | 577 | | | Yamada, 2001 |
| | | A24 | | NYSVRYRPGL | 578 | | | Yamada, 2001 |
| | | A24 | | LYAWEPSFL | 579 | | | Yamada, 2001 |
| MUC1 | glandular | A2 | 44 | STAPPVHNV | 580 | 950-958 | peptide | Brossart, 1999 |
| | epithelia | A2 | 44 | LLLLTVLTV | 581 | 20-Dec | peptide | Brossart, 1999 |
| | | A11 | | STAPPAHGV | 582 | | | Domenech, 1995 |
| | | A68 | | DVTSAPDNK | 583 | | | Kapp, 2009 |
| | | B7 | | VPGWGIALL | 584 | | | Kapp, 2009 |
| | | B7 | | DPSTDYYQEL | 585 | | | Kapp, 2009 |
| | | B44 | | TEAASRYNL | 586 | | | Kapp, 2009 |
| | | DR3 | 21 | PGSTAPPAHGVT | 587 | repeated | peptide | Hiltbold, 1998 |
| | | | | | | region | | |
| MUC5AC | surface | A24 | 20 | TCQPTCRSL | 588 | 716-724 | peptide | Yamazoe, 2011 |
| | mucosal cells, | | | | | | | |
| | respiratory | | | | | | | |
| | tract, and | | | | | | | |
| | stomach | | | | | | | |
| | epithelia | | | | | | | |
| Nucleophosmin | | A1 | | GCELKADKDY | 589 | | | Swoboda, 2010 |
| P15 | | A24 | | AYGLDFYIL | 590 | | | Robbins, 1995 |
| p53 | ubiquitous | A2 | 44 | LLGRNSFEV | 591 | 264-272 | peptide | Ropke, 1996 |
| | (low level) | A2 | 44 | RMPEAAPPV | 592 | 65-73 | peptide | Barfoed, 2000 |
| | | A2 | | LLPENNVLSPV | 593 | | | Keogh, 2001 |
| | | A2 | | SLPPPGTRV | 594 | | | |
| | | A2 | | VVPCEPPEV | 84 | | | Ito, 2007 |
| | | A2 | | YLGSYGFRL | 595 | | | |
| | | A2 | | SMPPPGTRV | 596 | | | Keogh, 2001 |
| | | A2 | | GLAPPQHLIRV | 597 | | | |
| | | A2 | | KLCPVQLWV | 598 | | | Keogh, 2001 |
| | | A2 | | KTCPVQLWV | 599 | | | Wurtzen, 2002 |
| | | A24 | | AIYKQSQHM | 600 | | | Umano, 2001 |
| | | B46 | 0.1 | SQKTYQGSY | 601 | 99-107 | autologous | Azuma, 2003 |
| | | | | | | | tumor cells | |
| | | Cw7 | | TRVLAMAIY | 602 | | | Ichiki, 2004 |
| | | DP5 | 3 | PGTRVRAMAIYKQ | 603 | 153-165 | peptide | Fujita, 1998 |
| | | DR14 | 6 | HLIRVEGNLRVE | 604 | 193-204 | peptide | Fujita, 1998 |
| PAK2 | | A2 | | KLAKPLSSL | 605 | | | Li, 2006 |
| | | A2 | | VLLGMEGSV | 606 | | | Li, 2006 |
| Papillomavirus binding | | A2 | | ALPSFQIPV | 607 | | | Tsukahara, 2009 |
| PAX3 | | A2 | | QLMAFNHLV | 608 | | | Rodeberg, 2006 |
| PAX5 | hemopoietic | A2 | 44 | TLPGYPPHV | 609 | 311-319 | peptide | Yan, 2008 |
| | system | | | | | | | |
| PBF | ovary, | B55 | 4 | CTACRWKKACQR | 610 | 499-510 | autologous | Tsukahara, 2004 |
| | pancreas, | | | | | | tumor cells | |
| | spleen, liver | | | | | | | |
| PGK1 | | A2 | | IIGGGMAFT | 611 | | | Shichijo, 2004 |
| PRAME | testis, ovary, | A2 | 44 | VLDGLDVLL | 612 | 100-108 | peptide | Kessler, 2001 |
| | endometrium, | A2 | 44 | SLYSFPEPEA | 613 | 142-151 | peptide | Kessler, 2001 |
| | adrenals | A2 | 44 | ALYVDSLFFL | 614 | 300-309 | peptide | Kessler, 2001 |
| | | A2 | 44 | SLLQHLIGL | 615 | 425-433 | peptide | Kessler, 2001 |
| | | A24 | 20 | LYVDSLFFLc | 616 | 301-309 | autologous | Ikeda, 1997 |
| | | | | | | | tumor cells | |
| | | B52 | | GQHLHLETF | 617 | | | Kawahara, 2006 |
| PRDI-BF1 | | A2 | | FGLFPRLCPV | 618 | | | Lotz, 2005 |
| Preprocalcitonin | | A2 | | VLLQAGSLHA | 420 | | | EI Hage, 2008 |
| (ppCT) | | | | | | | | |
| Prostatic acid | | ALDVYN | | | | | | Olson, 2010 |
| | | GLL | | | | | | |
| | | (SEQ ID | | | | | | |
| | | NO: 8) | | | | | | |
| | | FLFLLFF | | | | | | Olson, 2010 |
| | | WL (SEQ | | | | | | |
| | | ID NO: | | | | | | |
| | | 9) | | | | | | |
| | | TLMSA | | | | | | Olson, 2010 |
| | | MTNL | | | | | | |
| | | (SEQ ID | | | | | | |
| | | NO: 10) | | | | | | |
| | | ILLWQPI | | | | | | Machlenkin, 2005 |
| | | PV (SEQ | | | | | | |
| | | ID NO: | | | | | | |
| | | 11) | | | | | | |
| | | YLPFRN | | | | | | Terasaki, 2009 |
| | | CRP | | | | | | |
| | | (SEQ ID | | | | | | |
| | | NO: 12) | | | | | | |
| | | YLPFRN | | | | | | Terasaki, 2009 |
| | | CRP | | | | | | |
| | | (SEQ ID | | | | | | |
| | | NO: 12) | | | | | | |
| PSMA | prostate, CNS, | A1 | | HSTNGVTRIY | 619 | | | Corman, 1998 |
| | liver | A2 | | VLAGGFFLL | 620 | | | Lu, 2002 |
| | | A24 | | LYSDPADYF | 621 | | | Horiguchi, 2003 |
| | | A24 | 20 | NYARTEDFF | 622 | 178-186 | peptide | Horiguchi, 2002 |
| RAGE-1 | retina | A2 | 44 | LKLSGVVRL | 623 | 352-360 | peptide | Oehlrich, 2005 |
| | | A2 | 44 | PLPPARNGGLg | 624 | 32-40 | peptide | Oehlrich, 2005 |
| | | B7 | 17 | SPSSNRIRNT | 625 | 20-Nov | autologous | Gaugler, 1996 |
| | | | | | | | tumor cells | |
| Ran | | A2 | | YMFDVTSRV | 626 | | | Li, 2009 |
| | | A2 | | IMFDVTSRV | 627 | | | Li, 2009 |
| | | A33 | | HPLVFHTNR | 628 | | | Azuma, 2004 |
| | | A33 | | IIMFDVTSR | 629 | | | Azuma, 2004 |
| RGS5 | heart, skeletal | A2 | 44 | LAALPHSCL | 630 | 13-May | peptide | Boss, 2007 |
| | muscle, | A3 | 22 | GLASFKSFLK | 631 | 74-83 | peptide | Boss, 2007 |
| | pericytes | B8 | | MAQKRIHAL | 632 | | | Flad, 2006 |
| Ribosomal | | A31 | | KNKRILMEH | 633 | | | Kuroda, 2010 |
| protein L19 | | | | | | | | |
| RhoC | ubiquitous | A3 | 22 | RAGLQVRKNK | 634 | 176-185 | peptide | Wenandy, 2008 |
| | (low level) | | | | | | | |
| RNF43 | | A2 | 44 | ALWPWLLMA(T) | 635 | 11-19(20) | peptide | Uchida, 2004 |
| | | A24 | 20 | NSQPVWLCL | 636 | 721-729 | peptide | Uchida, 2004 |
| RU2AS | testis, kidney, | B7 | 17 | LPRWPPPQL | 637 | antisense | autologous | Van Den Eynde, |
| | bladder | | | | | | tumor cells | 1999 |
| SART-2 | | A24 | | DYSARWNEI | 638 | | | Nakao, 2000 |
| | | A24 | | AYDFLYNYL | 639 | | | Nakao, 2000 |
| | | A24 | | SYTRLFLIL | 640 | | | Nakao, 2000 |
| secernin 1 | ubiquitous | A2 | 44 | KMDAEHPEL | 641 | 196-204 | peptide | Suda, 2006 |
| SH3GLB2 | | A2 | | FLTPLRNFL | 642 | | | Fasso, 2008 |
| SOX4 | | Cw*1402 | | | | | | Friedman, 2004 |
| SOX10 | ubiquitous | A2 | 44 | AWISKPPGV | 643 | 332-340 | tumor- | Khong, 2002 |
| | (low level) | | | | | | infiltrating | |
| | | | | | | | lymphocytes | |
| | | A2 | 44 | SAWISKPPGV | 644 | 331-340 | tumor- | Khong, 2002 |
| | | | | | | | infiltrating | |
| | | | | | | | lymphocytes | |
| SPARC | | A24 | | MYIFPVHWQF | 645 | | | Inoue M, 2010 |
| | | A24 | | DYIGPCKYI | 646 | | | Inoue M, 2010 |
| STAT1- | | A2 | | KLQELNYNL | 647 | | | Ramakrishna, 2003 |
| alpha/beta | | | | | | | | |
| STEAP1 | prostate | A2 | 44 | MIAVFLPIV | 648 | 292-300 | peptide | Rodeberg, 2005 |
| | | A2 | | FLYTLLREV | 649 | | | Alves 2006 |
| | | A2 | | LLLGTIHAL | 650 | | | Machlenkin, 2005 |
| | | A2 | 44 | HQQYFYKIPILVINK | 651 | 102-116 | peptide | Kobayashi, 2007 |
| survivin | ubiquitous | A2 | 44 | ELTLGEFLKL | 652 | 95-104 | peptide/ | Schmitz, 2000 |
| | | | | | | | protein | Schmidt, 2003 |
| | | A2 | | QMFFCFKEL | 653 | | | Ciesielski, 2010 |
| | | A2 | | LMLGEFLKL | 654 | | | Andersen, 2001 |
| | | A2 | | TLPPAWQPFL | 655 | | | Schmitz, 2000 |
| | ubiquitous | DR1 | 18 | TLGEFLKLDRERAKN | 656 | 97-111 | peptide/ | Widenmeyer, 2012 |
| | | | | | | | protein | |
| survivin-2B | | A24 | | AYACNTSTL | 657 | | | Hirohashi, 2002 |
| Telomerase | testis, thymus, | A2 | 44 | ILAKFLHWL | 658 | 540-548 | peptide | Vonderheide, 1999 |
| | bone marrow, | A2 | 44 | RLVDDFLLV | 659 | 865-873 | peptide | Minev, 2000 |
| | lymph nodes | A2 | | LLTSRLRFI | 660 | | | Oslo, unpublished |
| | | | | | | | | data |
| | | A2 | | RLFFYRKSV | 661 | | | Hernandez, 2002 |
| | | A3 | | KLFGVLRLK | 662 | | | Vonderheide, 2001 |
| | | DR7 | 25 | RPGLLGASVLGLDDI | 663 | 672-686 | peptide | Schroers, 2002 |
| | | DR11 | 25 | LTDLQPYMRQFVAHL | 664 | 766-780 | peptide | Schroers, 2003 |
| Tie2 | | A2 | | FLPATLTMV | 665 | | | Ramage, 2004 |
| Topoisomerase | | A2 | | FLYDDNQRV | 666 | | | Ramakrishna, 2003 |
| II | | | | | | | | |
| TRG | | B52 | | YQLCLTNIF | 667 | | | Ohkouchi, 2003 |
| | | B62 | | YQLCLTNIF | 667 | | | Ohkouchi, 2003 |
| TPBG | multiple | A2 | 44 | RLARLALVL | 253 | 17-25 | peptide | Tykodi, 2012 |
| | tissues | | | | | | | |
| | (esophagus, | | | | | | | |
| | bladder, . . . ) | | | | | | | |
| TYMS | | A2 | | LMALPPCHAL | 668 | | | Shichijo, 2004 |
| VEGF | ubiquitous | B27 | 7 | SRFGGAVVR | 669 | -i | peptide | Weinzierl, 2008 |
| | (low level) | | | | | | | |
| VEGFR2/KDR | | A2 | | FLSTLTIDGV | 670 | | | Sun, 2006 |
| WHSC2 | | A26 | | ASLDSDPWV | 671 | | | Niu, 2009 |
| WNK2/ppMAP | | A26 | | DLLSHAFFA | 672 | | | Niu, 2009 |
| kkk | | | | | | | | |
| WT1 | testis, ovary, | A1 | 26 | TSEKRPFMCAY | 673 | 317-327 | peptide | Asemissen, 2006 |
| | bone marrow, | A2 | | RMFPNAPYL | 674 | | | Oka, 2000 |
| | spleen | A2 | | YMFPNAPYL | 675 | | | May, 2007 |
| | | A24 | 20 | CMTWNQMNL | 676 | 235-243 | peptide | Ohminami, 2000 |
| | | A24 | | CYTWNQMNL | 677 | | | Tsuboi, 2002 |
| | | A24 | | RWPSCQKKF | 678 | | | Azuma, 2002 |
| | | DP5 | 3 | LSHLQMHSRKH | 679 | 337-347 | peptide | Guo, 2005 |
| | | DP5 | 3 | KRYFKLSHLQMHSR | 680 | 332-347 | peptide | Lin, 2013 |
| | | | | KH | | | | |
| | | DR4 | 24 | KRYFKLSHLQMHSR | 680 | 332-347 | peptide | Fujiki, 2007 |
| | | | | KH | | | | |
| XBP1 | | A2 | | LLSGQPASA | 681 | | | Lotz, 2005 |
| CD33 | | | | | | | | |
| CD123 | | | | | | | | |
| CD38 | | | | | | | | |
| gastrin-17 | | | | | | | | |
| guanylyl | | | | | | | | |
| cyclase C | | | | | | | | |

Other

| | | | | | |
|---|---|---|---|---|---|
| HNRPL | A26 | NVLHFFNAPL | 682 | | Niu, 2009 |
| EHD2 | A3 | KLPNSVLGR | 683 | | Dorrschuck, 2004 |
| 707-AP | A2 | RVAALARDAP | 684 | | Morioka, 1995 |
| mAb MF11-30 | A2 | LLVLLYSKL | 685 | | Murray, 2004 |
| VH | | | | | |
| Replication | A2 | YLMDTSGKV | 686 | | Ramakrishna, 2003 |
| protein A | | | | | |
| RU1 | B51 | VPYGSFKHV | 687 | | Morel, 2000 |
| septin 2, | A2 | RLYPWGVVEV | 688 | | Ramakrishna, 2003 |
| Nedd5 | | | | | |
| SGT1B | B39 | CHILLGNYC | 689 | | So, 2005 |
| MOG | | MEVGWYRSPFSRV | 690 | 35-55 | |
| | | VHLYRNGK | | | |

Virus Related Epitopes

| | | | | |
|---|---|---|---|---|
| Epstein Barr | A2 | CLGGLLTMV | 691 | |
| Virus | A2 | GLCTLVAML | 692 | |
| | A2 | FLYALALLL | 693 | |
| | A2 | YVLDHLIVV | 694 | |
| | A3 | RLRAEAQVK | 695 | |
| | A11 | AVFDRKSDAK | 696 | |
| | B7 | RPPIFIRLL | 697 | |
| | A2 | VLQWASLAV | 698 | |
| | | FMVFLQTHI | 699 | |
| | | FLQTHIFAEV | 700 | |
| | | SIVCYFMVFL | 701 | |
| Cytomegalo- | A1 | YSEHPTFTSQY | 702 | |
| virus | A1 | VTEHDTLLY | 703 | |
| | A2 | NLVPMVATV | 704 | |
| | A2 | VLEETSVML | 705 | |
| | A3 | TTVYPPSSTAK | 706 | |
| | A11 | GPISGHVLK | 707 | |
| | B7 | TPRVTGGGAM | 708 | |
| | B7 | RPHERNGFTV | 709 | |
| Influenza virus | A1 | VSDGGPNLY | 710 | |
| | A2 | GILGFVFTL | 711 | |
| Human | A2 | TIHDIILECV | 712 | 29-38 |
| papilloma virus | A2 | YMLDLQPET | 713 | 11-19 |
| | A2 | YMLDLQPETT | 714 | 11-20 |

In other embodiments, the antigenic polypeptide is an antigenic polypeptide from any one of the antigens disclosed herein. For example, in some embodiments the antigenic polypeptide is an antigenic polypeptide from an antigen selected from the antigens disclosed in Tables 1 and 14-24. In some embodiments, the antigenic polypeptide is an antigenic polypeptide from an antigen selected from the antigens disclosed in Table 16. In some embodiments, the antigenic polypeptide is an antigenic polypeptide from an antigen selected from the antigens disclosed in Table 17. In some embodiments, the antigenic polypeptide is an antigenic polypeptide from an antigen selected from the antigens disclosed in Table 18. In some embodiments, the antigenic polypeptide is an antigenic polypeptide from an antigen selected from the antigens disclosed in Table 19. In some embodiments, the antigenic polypeptide is an antigenic polypeptide from an antigen selected from the antigens disclosed in Table 20. In some embodiments, the antigenic polypeptide is an antigenic polypeptide from an antigen selected from the antigens disclosed in Table 21. In some embodiments, the antigenic polypeptide is an antigenic polypeptide from an antigen selected from the antigens disclosed in Table 22 In some embodiments, the antigenic polypeptide is an antigenic polypeptide from an antigen selected from the antigens disclosed in Table 23 In some embodiments, the antigenic polypeptide is an antigenic polypeptide from an antigen selected from the antigens disclosed in Table 24.

An exemplary antigenic polypeptide, e.g. a human polypeptide, selected from Table 1, or from Tables 14-24 includes:

a) a naturally occurring form of the human polypeptide, e.g., a naturally occurring form of the human polypeptide that is not associated with a disease state;

b) the human polypeptide having a sequence appearing in a database, e.g., GenBank database, on Dec. 22, 2017, for example a naturally occurring form of the human polypeptide that is not associated with a disease state having a sequence appearing in a database, e.g., GenBank database, on Dec. 22, 2017;

c) a human polypeptide having a sequence that differs by no more than 1, 2, 3, 4, 5 or 10 amino acid residues from a sequence of a) or b);

d) a human polypeptide having a sequence that differs at no more than 1, 2, 3, 4, 5 or 10% its amino acids residues from a sequence of a) or b);

e) a human polypeptide having a sequence that does not differ substantially from a sequence of a) or b); or

f) a human polypeptide having a sequence of c), d), or e) that does not differ substantially in a biological activity, e.g., an enzymatic activity (e.g., specificity or turnover) or binding activity (e.g., binding specificity or affinity) from a human polypeptide having the sequence of a) or b). Candidate peptides under f) can be made and screened for similar activity as described herein and would be equivalent hereunder if expressed in engineered erythroid cells as described herein).

In embodiments, an exogenous antigenic polypeptide comprises a human polypeptide or fragment thereof, e.g., all or a fragment of a human polypeptide of a), b), c), d), e), or f) of the preceding paragraph. In an embodiment, the exogenous polypeptide comprises a fusion polypeptide comprising all or a fragment of a human polypeptide of a), b), c), d), e), or f) of the preceding paragraph and additional amino acid sequence. In an embodiment the additional amino acid sequence comprises all or a fragment of human polypeptide of a), b), c), d), e), or f) of the preceding paragraph for a different human polypeptide.

In certain embodiments, the exogenous antigenic polypeptides are presented on antigen-presenting polypeptides, e.g., the exogenous antigenic polypeptide is specifically bound to the exogenous antigen-presenting polypeptides, e.g. histocompatibility molecules (MHCI or MHCII).

In some embodiments, the exogenous antigenic polypeptide is 8 amino acids in length to 24 amino acids in length, for example 8, 9, 10, 11, 12, 13, 14, 15, 16, 17, 18, 19, 20, 21, 22, 23, or 24 amino acids in length. In further embodiments, a cleavable site is introduced into the exogenous antigenic polypeptide.

MAGE-A

MAGE-A antigens are expressed in a variety of cancers of diverse histological origin and germinal cells. MAGE-A antigens belong to the larger family of cancer/testis antigens (CTA), whose expression is consistently detected in cancers of different histological origin and germinal cells (Simpson et al. Nat Rev Cancer. 2005 August; 5(8):615-25). The MAGE-A gene family has 12 members (MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12) located on chromosome Xq28 (Chomez et al., Cancer Res. 2001 Jul. 15; 61(14):5544-51; DePlaen et al., Immunogenetics. 1994; 40(5):360-9). MAGE-A1, -A2, -A3, -A4, -A6, -A10, and -A12 are expressed in a significant proportion of primary and metastatic tumors of various histological types and are targets of tumor antigen-specific cytotoxic T lymphocytes. Individual MAGE-A expression varies from one tumor type to the other but, overall, the large majority of tumors express at least one MAGE-A antigen. Specific gene products have been identified by immunohistochemistry in cancers of different histological origin, including high percentages of non-small cell lung cancers (NSCLC), bladder cancers, esophageal and head and neck cancers, myeloma, sarcomas, and triple negative breast cancers (Juretic et al. Lancet Oncol. 2003 February; 4(2):104-9; Curigliano et al., Ann Oncol. 2011 January; 22(1):98-103; vanBaren et al., Ann Oncol. 2011 January; 22(1):98-103; Antonescu et al., Hum Pathol. 2002 February; 33(2):225-9).

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A antigen. In a further embodiment, the MAGE-A antigen is selected from MAGE-A1, MAGE-A2, MAGE-A3, MAGE-A4, MAGE-A5, MAGE-A6, MAGE-A7, MAGE-A8, MAGE-A9, MAGE-A10, MAGE-A11 and MAGE-A12. In some embodiments, the erythroid cell is an enucleated cell. In some embodiments, the erythroid cell is a nucleated cell.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A1 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A2 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A3 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A4 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A5 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A6 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A7 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A8 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A9 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A10 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A11 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A12 antigen.

In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is a MAGE-A antigen. In some embodiments, the MAGE-A antigen is selected from MAGE-A1, MAGE-A2, MAGE-A3, MAGE-A4, MAGE-A5, MAGE-A6, MAGE-A7, MAGE-A8, MAGE-A9, MAGE-A10, MAGE-A11 and MAGE-A12. In some embodiments, the MAGE-A antigen is selected from MAGE-A1, MAGE-A2, MAGE-A3, MAGE-A4, MAGE-A6, MAGE-A10 and MAGE-A12.

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is a MAGE-A1 antigen. In some embodiments, the MAGE-A1 antigen is selected from the group consisting of EADPTGHSY (SEQ ID NO: 123), KVLEYVIKV (SEQ ID NO: 126), SLFRAVITK (SEQ ID NO: 127), EVYDGREHSA (SEQ ID NO: 129), RVRFFFPSL (SEQ ID NO: 130), EADPTGHSY (SEQ ID NO: 123), REPVTKAEML (SEQ ID NO: 131), KEADPTGHSY (SEQ ID NO: 132), DPARYEFLW (SEQ ID NO: 133), ITKKVADLVGF (SEQ ID NO: 134), SAFPTTINF (SEQ ID NO: 135), SAYGEPRKL (SEQ ID NO: 136), RVRFFFPSL (SEQ ID NO: 130), SAYGEPRKL (SEQ ID NO: 136), TSCILESLFRAVITK (SEQ ID NO: 137), PRALAETSYVKVLEY (SEQ ID NO: 138), FLLLKYRAREPVTKAE (SEQ ID NO: 139) and EYVIKVSARVRF (SEQ ID NO: 140).

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein, the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II single chain fusion, and wherein the exogenous antigenic polypeptide is a MAGE-A2 antigen. In some embodiments, the MAGE-A2 antigen is selected from the group consisting of YLQLVFGIEV (SEQ ID NO: 141), EYLQLVFGI (SEQ ID NO: 145), REPVTKAEML (SEQ ID NO: 131), EGDCAPEEK (SEQ ID NO: 146) and LLKYRAREPVTKAE (SEQ ID NO: 147).

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, and wherein the exogenous antigenic polypeptide is a MAGE-A3 antigen. In some embodiments, the MAGE-A3 antigen is selected from the group consisting of EVDPIGHLY (SEQ ID NO: 148), FLWGPRALVD (SEQ ID NO: 149), KVAELVHFL (SEQ ID NO: 150), TFPDLESEF (SEQ ID NO: 153), VAELVHFLL (SEQ ID NO: 154), MEVDPIGHLY (SEQ ID NO: 155), EVDPIGHLY (SEQ ID NO: 148), REPVTKAEML (SEQ ID NO: 131), AELVHFLLLI (SEQ ID NO: 157), EVDPIGHLY (SEQ ID NO: 148), WQYFFPVIF (SEQ ID NO: 158), EGDCAPEEK (SEQ ID NO: 146), KKLLTQHFVQENYLEY (SEQ ID NO: 159), RKVAELVHFLLLKYR (SEQ ID NO: 160), KKLLTQHFVQENYLEY (SEQ ID NO: 159), ACYEFLWGPRALVETS (SEQ ID NO: 161), RKVAELVHFLLLKYR (SEQ ID NO: 160), VIFSKASSSLQL (SEQ ID NO: 162), VIFSKASSSLQL (SEQ ID NO: 162), VFGIELMEVDPIGHL (SEQ ID NO: 163), GDNQIMPKAGLLIIV (SEQ ID NO: 164), TSYVKVLHHMVKISG (SEQ ID NO: 165), RKVAELVHFLLLKYRA (SEQ ID NO: 166) and FLLLKYRAREPVTKAE (SEQ ID NO: 139).

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, and wherein the exogenous antigenic polypeptide is a MAGE-A4 antigen. In embodiments, the MAGE-A4 antigen is selected from the group consisting of EVDPASNTYJ (SEQ ID NO: 167) and GVYDGREHTV (SEQ ID NO: 168).

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, and wherein the exogenous antigenic polypeptide is a MAGE-A5 antigen.

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, and wherein the exogenous antigenic polypeptide is a MAGE-A6 antigen. In some embodiments, the MAGE-A6 antigen is selected from the group consisting of SESLKMIF (SEQ ID NO: 170), MVKISGGPR (SEQ ID NO: 171), EVDPIGHVY (SEQ ID NO: 172), REPVTKAEML (SEQ ID NO: 131), EGDCAPEEK (SEQ ID NO: 146), ISGGPRISY (SEQ ID NO: 173), LLKYRAREPVTKAE (SEQ ID NO: 147).

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, wherein the exogenous antigenic polypeptide is a MAGE-A7 antigen.

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, wherein the exogenous antigenic polypeptide is a MAGE-A8 antigen.

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, wherein the exogenous antigenic polypeptide is a MAGE-A9 antigen (SEQ ID NO: 176). In some embodiments, the MAGE-A9 antigen is ALSVMGVYV.

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, wherein the exogenous antigenic polypeptide is a MAGE-A10 antigen. In some embodiments, the MAGE-A10 antigen is selected from the group consisting of GLYDGMEHLI (SEQ ID NO: 715) and DPARYEFLW (SEQ ID NO: 133).

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, wherein the exogenous antigenic polypeptide is a MAGE-A11 antigen.

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, wherein the exogenous antigenic polypeptide is a MAGE-A12 antigen. In some embodiments, the MAGE-A12 antigen is selected from the group consisting of FLWGPRALVE (SEQ ID NO: 179), VRIGHLYIL (SEQ ID NO: 180), EGDCAPEEK (SEQ ID NO: 146), REPFTKAEMLGSVIR (SEQ ID NO: 181) and AELVHFLLLKYRAR (SEQ ID NO: 182).

Also encompassed by the disclosure is an artificial antigen presenting cell (aAPC) comprising an erythroid cell, wherein the erythroid cell presents, e.g. comprises on the cell surface, at least one exogenous antigenic polypeptide that comprises an epitope common to several tumor antigens of the MAGE-A family. In some embodiments, the exogenous antigenic polypeptide comprises the epitope p248v9 (YLEYRQVPV (SEQ ID NO: 124)), an immunogenic peptide presented by HLA-A*0201 and capable of inducing cytotoxic T lymphocytes (CTLs) which recognize all the MAGE-A antigens. In some embodiments, the exogenous antigenic polypeptide comprises the epitope p248g9 (YLEYRQVPG (SEQ ID NO: 156)), an immunogenic peptide which is capable of inducing CTLs which recognize MAGE-A2, A3, A4, A6, A10, A12. In some embodiments, the exogenous antigenic polypeptide comprises the epitope p248d9 (YLEYRQVPD (SEQ ID NO: 125)), an immunogenic peptide which is capable of inducing CTLs which recognize MAGE-A1.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is p248v9 (YLEYRQVPV (SEQ ID NO: 124)). In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is p248g9 (YLEYRQVPG (SEQ ID NO: 156)). In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is p248d9 (YLEYRQVPD (SEQ ID NO: 125)).

Also encompassed by the disclosure is an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, and wherein the exogenous antigenic polypeptide is p248v9 (YLEYRQVPV (SEQ ID NO: 124)). In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, and wherein the exogenous antigenic polypeptide is p248g9 (YLEYRQVPG (SEQ ID NO: 156)). In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptides, e.g. histocompatibility molecules (MHCI, MHCII), wherein the exogenous antigen-presenting polypeptide is an MHC class I polypeptide or single chain fusion or an MHC class II polypeptide or single chain fusion, and wherein the exogenous antigenic polypeptide is p248d9 (YLEYRQVPD (SEQ ID NO: 125)). In embodiments, the exogenous antigen-presenting polypeptide is MHC I HLA-A, e.g, MHC I HLA-A *201.

In one particular embodiment, an artificial antigen presenting cell comprises an erythroid cell, wherein the erythroid cell presents, e.g. comprises on the cell surface, at least one exogenous antigenic polypeptide, p248v9 (YLEYRQVPV (SEQ ID NO: 124)), fused to an exogenous antigen presenting polypeptide, MHCI HLA-A *201, fused to the GPA transmembrane domain (GPA). In one particular embodiment, an artificial antigen presenting cell comprises an erythroid cell, wherein the erythroid cell presents, e.g. comprises on the cell surface, at least one exogenous antigenic polypeptide, p248g9 (YLEYRQVPG (SEQ ID NO: 156)), fused to an exogenous antigen presenting polypeptide, MHCI HLA-A *201, fused to the GPA transmembrane domain (GPA). In one particular embodiment, an artificial antigen presenting cell comprises an erythroid cell, wherein the erythroid cell presents, e.g. comprises on the cell surface, at least one exogenous antigenic polypeptide, p248d9 (YLEYRQVPD (SEQ ID NO: 125)), fused to an exogenous antigen presenting polypeptide, MHCI HLA-A *201, fused to the GPA transmembrane domain (GPA).

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A antigen as listed in Table 1. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A antigen as listed in Table 1, and further presents, e.g. comprises on the cell surface, an exogenous polypeptide comprising 4-1BBL. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A antigen selected from the MAGE-A antigens listed in Table 1, and further presents, e.g. comprises on the cell surface, an exogenous antigen-presenting polypeptide, wherein the exogenous antigenic polypeptide is specifically bound to the exogenous antigen-presenting polypeptide, wherein the exogenous antigen-presenting polypeptide is the corresponding MHC Class I or MHC Class II HLA listed in Table 1 for the particular MAGE-A antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A antigen selected from the MAGE-A antigens listed in Table 1, and further presents, e.g. comprises on the cell surface, an exogenous antigen-presenting polypeptide, wherein the exogenous antigenic polypeptide is specifically bound to the exogenous antigen-presenting polypeptide, wherein the exogenous antigen-presenting polypeptide is the corresponding MHC Class I or MHC Class II HLA listed in Table 1 for the particular MAGE-A antigen, and an exogenous polypeptide comprising 4-1BBL. In another embodiment, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MAGE-A antigen selected from the MAGE-A antigens listed in Table 1, and further presents, e.g. comprises on the cell surface, an exogenous antigen-presenting polypeptide, wherein the exogenous antigenic polypeptide is specifically bound to the exogenous antigen-presenting polypeptide, wherein the exogenous antigen-presenting polypeptide is the corresponding MHC Class I HLA listed in Table 1 for the particular MAGE-A antigen, and an exogenous polypeptide comprising 4-1BBL.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide comprises or consist of a MAGE-A antigen selected from EADPTGHSY (SEQ ID NO: 123), KVLEYVIKV (SEQ ID NO: 126), SLFRAVITK (SEQ ID NO: 127), EVYDGREHSA (SEQ ID NO: 129), RVRFFFPSL (SEQ ID NO: 130), EADPTGHSY (SEQ ID NO: 123), REPVTKAEML (SEQ ID NO: 131), KEADPTGHSY (SEQ ID NO: 132), DPARYEFLW (SEQ ID NO: 133), ITKKVADLVGF (SEQ ID NO: 134), SAFPTTINF (SEQ ID NO: 135), SAYGEPRKL (SEQ ID NO: 136), RVRFFFPSL (SEQ ID NO: 130), SAYGEPRKL (SEQ ID NO: 136), TSCILESLFRAVITK (SEQ ID NO: 137), PRALAETSYVKVLEY (SEQ ID NO: 138), FLLLKYRAREPVTKAE (SEQ ID NO: 139), EYVIKVSARVRF (SEQ ID NO: 140), YLQLVFGIEV (SEQ ID NO: 141), EYLQLVFGI (SEQ ID NO: 145), REPVTKAEML (SEQ ID NO: 131), EGDCAPEEK (SEQ ID NO: 146), LLKYRAREPVTKAE (SEQ ID NO: 147), EVDPIGHLY (SEQ ID NO: 148), FLWGPRALVD (SEQ ID NO: 149), KVAELVHFL (SEQ ID NO: 150), TFPDLESEF (SEQ ID NO: 153), VAELVHFLL MEVDPIGHLY (SEQ ID NO: 716), EVDPIGHLY (SEQ ID NO: 148), REPVTKAEML (SEQ ID NO: 131), AELVHFLLLI (SEQ ID NO: 157), MEVDPIGHLY (SEQ ID NO: 155), WQYFFPVIF (SEQ ID NO: 158), EGDCAPEEK (SEQ ID NO: 146), KKLLTQHFVQENYLEY (SEQ ID NO: 159), RKVAELVHFLLLKYR (SEQ ID NO: 160), KKLLTQHFVQENYLEY (SEQ ID NO: 159), ACYEFLWGPRALVETS (SEQ ID NO: 161), RKVAELVHFLLLKYR (SEQ ID NO: 160), VIFSKASSSLQL (SEQ ID NO: 162), VIFSKASSSLQL (SEQ ID NO: 162), VFGIELMEVDPIGHL (SEQ ID NO: 163), GDNQIMPKAGLLIIV (SEQ ID NO: 164), TSYVKVLHHMVKISG (SEQ ID NO: 165), RKVAELVHFLLLKYRA (SEQ ID NO: 166), FLLLKYRAREPVTKAE (SEQ ID NO: 139), EVDPASNTYj (SEQ ID NO: 167), GVYDGREHTV (SEQ ID NO: 168), NYKRCFPVI (SEQ ID NO: 169), SESLKMIF (SEQ ID NO: 170), MVKISGGPR (SEQ ID NO: 171), EVDPIGHVY (SEQ ID NO: 172), REPVTKAEML (SEQ ID NO: 131), EGDCAPEEK (SEQ ID NO: 146), ISGGPRISY (SEQ ID NO: 173), LLKYRAREPVTKAE (SEQ ID NO: 147), ALSVMGVYV (SEQ ID NO: 176), GLYDGMEHLI (SEQ ID NO: 715), DPARYEFLW (SEQ ID NO: 133), FLWGPRALVE (SEQ ID NO: 179), VRIGHLYIL (SEQ ID NO: 180), EGDCAPEEK (SEQ ID NO: 146), REPFTKAEMLGSVIR (SEQ ID NO: 181) and AELVHFLLLKYRAR (SEQ ID NO: 182).

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide comprises or consists of a MAGE-A antigen selected from EADPTGHSY (SEQ ID NO: 123), KVLEYVIKV (SEQ ID NO: 126), SLFRAVITK (SEQ ID NO: 127), EVYDGREHSA (SEQ ID NO: 129), RVRFFFPSL (SEQ ID NO: 130), EADPTGHSY (SEQ ID NO: 123), REPVTKAEML (SEQ ID NO: 131), KEADPTGHSY (SEQ ID NO: 132), DPARYEFLW (SEQ ID NO: 133), ITKKVADLVGF (SEQ ID NO: 134), SAFPTTINF (SEQ ID NO: 135), SAYGEPRKL (SEQ ID NO: 136), RVRFFFPSL (SEQ ID NO: 130), SAYGEPRKL (SEQ ID NO: 136), TSCILESLFRAVITK (SEQ ID NO: 137), PRALAETSYVKVLEY (SEQ ID NO: 138), FLLLKYRAREPVTKAE (SEQ ID NO: 139), EYVIKVSARVRF (SEQ ID NO: 140), YLQLVFGIEV (SEQ ID NO: 141), EYLQLVFGI (SEQ ID NO: 145), REPVTKAEML (SEQ ID NO: 131), EGDCAPEEK (SEQ ID NO: 146), LLKYRAREPVTKAE (SEQ ID NO: 147), EVDPIGHLY (SEQ ID NO: 148), FLWGPRALVD (SEQ ID NO: 149), KVAELVHFL (SEQ ID NO: 150), TFPDLESEF (SEQ ID NO: 153), VAELVHFLL MEVDPIGHLY (SEQ ID NO: 716), EVDPIGHLY (SEQ ID NO: 148), REPVTKAEML (SEQ ID NO: 131), AELVHFLLLI (SEQ ID NO: 157), MEVDPIGHLY (SEQ ID NO: 155), WQYFFPVIF (SEQ ID NO: 158), EGDCAPEEK (SEQ ID NO: 146), KKLLTQHFVQENYLEY (SEQ ID NO: 159), RKVAELVHFLLLKYR (SEQ ID NO: 160), KKLLTQHFVQENYLEY (SEQ ID NO: 159), ACYEFLWGPRALVETS (SEQ ID NO: 161), RKVAELVHFLLLKYR (SEQ ID NO: 160), VIFSKASSSLQL (SEQ ID NO: 162), VIFSKASSSLQL (SEQ ID NO: 162), VFGIELMEVDPIGHL (SEQ ID NO: 163), GDNQIMPKAGLLIIV (SEQ ID NO: 164), TSYVKVLHHMVKISG (SEQ ID NO: 165), RKVAELVHFLLLKYRA (SEQ ID NO: 166), FLLLKYRAREPVTKAE (SEQ ID NO: 139), EVDPASNTYj (SEQ ID NO: 167), GVYDGREHTV (SEQ ID NO: 168), NYKRCFPVI (SEQ ID NO: 169), SESLKMIF (SEQ ID NO: 170), MVKISGGPR (SEQ ID NO: 171), EVDPIGHVY (SEQ ID NO: 172), REPVTKAEML (SEQ ID NO: 131), EGDCAPEEK (SEQ ID NO: 146), ISGGPRISY (SEQ ID NO: 173), LLKYRAREPVTKAE (SEQ ID NO: 147), ALSVMGVYV (SEQ ID NO: 176), GLYDGMEHLI (SEQ ID NO: 715), DPARYEFLW (SEQ ID NO: 133), FLWGPRALVE (SEQ ID NO: 179), VRIGHLYIL (SEQ ID NO: 180), EGDCAPEEK (SEQ ID NO: 146), REPFTKAEMLGSVIR (SEQ ID NO: 181) and AELVHFLLLKYRAR (SEQ ID NO: 182), and wherein the erythroid cell further comprises an exogenous polypeptide comprising 4-1BBL.

In a further embodiment, an aAPC as described herein, comprising any of the exogenous antigenic polypeptides comprising a MAGE-A antigen (e.g. a MAGE-A1, MAGE-A2, MAGE-A3, MAGE-A4, MAGE-A5, MAGE-A6, MAGE-A7, MAGE-A8, MAGE-A9, MAGE-A10, MAGE-A11 or MAGE-A12 antigen, as set forth above), can be engineered to further comprise an exogenous polypeptide comprising 4-1BBL. In another further embodiment, an aAPC as described herein, comprising at least one exogenous antigenic polypeptide that comprises an epitope common to one or more MAGE-A antigens (e.g. p248v9, p248g9 and/or p248d9) as described herein, can be engineered to further comprise an exogenous polypeptide comprising 4-1BBL.

An aAPC as described herein, comprising any of the exogenous antigenic polypeptides comprising a MAGE-A antigen (e.g. a MAGE-A1, MAGE-A2, MAGE-A3, MAGE-A4, MAGE-A5, MAGE-A6, MAGE-A7, MAGE-A8, MAGE-A9, MAGE-A10, MAGE-A11 or MAGE-A12 antigen, as set forth above) can be used in the treatment of cancer, as described in more detail below. An aAPC as described herein, comprising any of the exogenous antigenic polypeptides comprising a MAGE-A antigen (e.g. a MAGE-A1, MAGE-A2, MAGE-A3, MAGE-A4, MAGE-A5, MAGE-A6, MAGE-A7, MAGE-A8, MAGE-A9, MAGE-A10, MAGE-A11 or MAGE-A12 antigen, as set forth above), and further comprising an exogenous polypeptide comprising 4-1BBL, can be used in the treatment of cancer, as described in more detail below. Further, an aAPC as described herein, comprising an exogenous antigenic polypeptide that comprises an epitope common to one or more MAGE-A antigens (e.g. p248v9, p248g9 and/or p248d9) can be used in the treatment of cancer, as described in more detail below. Further, an aAPC as described herein, comprising an exogenous antigenic polypeptide that comprises an epitope common to one or more MAGE-A antigens (e.g. p248v9, p248g9 and/or p248d9), and further comprising an exogenous polypeptide comprising 4-1BBL, can be used in the treatment of cancer, as described in more detail below.

### Neutrophil Granule Protease

Neutrophil elastase, proteinase 3, and cathepsin G are three homologous proteases that belong to the chymotrypsin superfamily of serine proteases. They act in combination with reactive oxygen species to help degrade engulfed microorganisms inside phagolysosomes. These proteases are also externalized in an active form during neutrophil activation at inflammatory sites, thus contributing to the regulation of inflammatory and immune responses. In addition to their involvement in pathogen destruction and the regulation of proinflammatory processes, neutrophil serine proteases (NSPs) are also involved in a variety of inflammatory human conditions, including chronic lung diseases (chronic obstructive pulmonary disease, cystic fibrosis, acute lung injury, and acute respiratory distress syndrome) and cancer. For example, proteinase 3 is highly expressed in acute myelogenous leukemia and in prostate cancer cells (Kolnin et al., Blood 2016 128:1025). Proteinasae 3 and neutrophil elastase have been shown to be aberrantly expressed in breast cancer cells (Desmedt et al. Int J Cancer, 2006 Dec. 1:119).

Neutrophil elastase is an enzyme that in humans is encoded by the ELANE gene. Neutrophil elastase is secreted by neutrophils and macrophages during inflammation, and it destroys bacteria and host tissue. Proteinase 3 is an enzyme that in humans is encoded by the PRTN3 gene. In human neutrophils, proteinase 3 contributes to the proteolytic generation of antimicrobial peptides. It is also the target of anti-neutrophil cytoplasmic antibodies (ANCAs) of the c-ANCA (cytoplasmic subtype) class, a type of antibody frequently found in the disease granulomatosis with polyangiitis. Cathepsin G is a protein that in humans is encoded by the CTSG gene. The encoded protease has a specificity similar to that of chymotrypsin C, and may participate in the killing and digestion of engulfed pathogens, and in connective tissue remodeling at sites of inflammation. In addition, the encoded protein is antimicrobial, with bacteriocidal activity against S. aureus and N. gonorrhoeae.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a neutrophil granule protease antigen. In a further embodiment, the neutrophil granule protease is selected from neutrophil elastase, proteinase 3 and cathepsin G. In some embodiments, the erythroid cell is an enucleated cell. In some embodiments, the erythroid cell is a nucleated cell.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a neutrophil elastase antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a proteinase 3 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a cathepsin G antigen. I

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a neutrophil elastase antigen, and wherein the erythroid cell further presents, e.g. comprises on the cell surface, at least one exogenous polypeptide comprising 4-1BBL. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a proteinase 3 antigen, and wherein the erythroid cell further presents, e.g. comprises on the cell surface, at least one exogenous polypeptide comprising 4-1BBL. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a cathepsin G antigen, and wherein the erythroid cell further presents, e.g. comprises on the cell surface, at least one exogenous polypeptide comprising 4-1BBL.

In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigen-presenting polypeptide is an MHC class I polypeptide or single chain fusion or an MHC class II polypeptide or single chain fusion, wherein the exogenous antigenic polypeptide is a neutrophil granule protease antigen. In a further embodiment, the neutrophil granule protease is selected from neutrophil elastase, proteinase 3 and cathepsin G.

In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is a neutrophil granule protease antigen, and wherein the erythroid cell further presents, e.g. comprises on the cell surface, at least one exogenous polypeptide comprising 4-1BBL. In a further embodiment, the neutrophil granule protease is selected from neutrophil elastase, proteinase 3 and cathepsin G.

In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the exogenous antigenic polypeptide is a neutrophil granule protease antigen, and wherein the erythroid cell further presents, e.g. comprises on the cell surface, at least one exogenous polypeptide comprising 4-1BBL. In a further embodiment, the neutrophil granule protease is selected from neutrophil elastase, proteinase 3 and cathepsin G.

In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is a neutrophil elastase antigen. In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is a proteinase 3 antigen. In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is a cathepsin G antigen.

PR1 (VLQELNVTV (SEQ ID NO: 225)) is an HLA-A2-restricted peptide derived from the myeloid proteins proteinase 3 and neutrophil elastase. PR1 is recognized on myeloid leukemia cells by cytotoxic T lymphocytes (CTLs) that preferentially kill leukemia and contribute to cytogenetic remission.

Accordingly, also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is PR1.

Also encompassed by the disclosure is an artificial antigen presenting cell (aAPC) engineered to activate T cells, wherein the aAPC comprises an erythroid cell, wherein the erythroid cell presents, e.g. comprises on the cell surface, at least one exogenous antigenic polypeptide, wherein the at least one exogenous antigenic polypeptide is PR1.

Also encompassed by the disclosure is an artificial antigen presenting cell (aAPC) engineered to activate T cells, wherein the aAPC comprises an erythroid cell, wherein the erythroid cell presents, e.g. comprises on the cell surface, at least one exogenous antigenic polypeptide, wherein the at least one exogenous antigenic polypeptide is PR1, and wherein the erythroid cell further presents, e.g. comprises on the cell surface, at least one exogenous polypeptide comprising 4-1BBL.

Also encompassed by the disclosure is an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is PR1. In embodiments, the exogenous antigen-presenting polypeptide is an MHC Class I HLA-A2 polypeptide or single chain fusion.

Also encompassed by the disclosure is an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is PR1, and wherein the erythroid cell further presents, e.g. comprises on the cell surface, at least one exogenous polypeptide comprising 4-1BBL. In embodiments, the exogenous antigen-presenting polypeptide is an MHC Class I HLA-A2 polypeptide or single chain fusion.

In one particular embodiment, an artificial antigen presenting cell comprises an erythroid cell, wherein the erythroid cell presents, e.g. comprises on the cell surface, at least one exogenous antigenic polypeptide, PR1, fused to an exogenous antigen presenting polypeptide, MHCI HLA-A2, fused to the GPA transmembrane domain (GPA).

In one particular embodiment, an artificial antigen presenting cell comprises an erythroid cell, wherein the erythroid cell presents, e.g. comprises on the cell surface, at least one exogenous antigenic polypeptide, PR1, fused to an exogenous antigen presenting polypeptide, MHCI HLA-A2, fused to the GPA transmembrane domain (GPA), and wherein the erythroid cell further presents, e.g. comprises on the cell surface, at least one exogenous polypeptide comprising 4-1BBL.

An aAPC as described herein, comprising any of the exogenous antigenic polypeptides comprising a neutrophil granule protease antigen (e.g. neutrophil elastase antigen, proteinase 3 antigen, or cathepsin G antigen) can be used in the treatment of cancer, as described in more detail below. An aAPC as described herein, comprising any of the exogenous antigenic polypeptides comprising a neutrophil granule protease antigen (e.g. neutrophil elastase antigen, proteinase 3 antigen, or cathepsin G antigen), and further comprising an exogenous polypeptide comprising 4-1BBL, can be used in the treatment of cancer, as described in more detail below. An aAPC as described herein, comprising an exogenous antigenic polypeptide comprising PR1 can be used in the treatment of cancer, as described in more detail below. An aAPC as described herein, comprising an exogenous antigenic polypeptide comprising PR1, and further comprising an exogenous polypeptide comprising 4-1BBL, can be used in the treatment of cancer, as described in more detail below.

NY-ESO-1/LAGE-2

Cancer/testis (C/T) antigens are a category of tumor antigens with normal expression restricted to male germ cells in the testis but not in adult somatic tissues. In some cases, CT antigens are also expressed in ovary and in trophoblast. In malignancy, this gene regulation is disrupted, resulting in CT antigen expression in a proportion of tumors of various types. Cancer/testis antigen 1 (also known as Autoimmunogenic Cancer/Testis Antigen NY-ESO-1 or LAGE-2) is a protein that in humans is encoded by the CTAG1B gene. Cancer-testis antigen NY-ESO-1, initially cloned by the SEREX (serological analysis of recombinant tumor cDNA expression libraries) approach from an esophageal cancer, elicits humoral and cellular immune responses in a high proportion of patients with NY-ESO-1-expressing cancers (Stockert et al., J. Exp. Med. 1998; 187:1349-1354; Jager et al. J. Exp. Med. 1998; 187:265-270).

In one aspect, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a NY-ESO-1/LAGE-2 antigen. In some embodiments, the erythroid cell is an enucleated erythroid cell. In some embodiments, the erythroid cell is a nucleated cell.

In one aspect, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a NY-ESO-1/LAGE-2 antigen, and wherein the erythroid cell further presents, e.g. comprises on the cell surface, at least one exogenous polypeptide comprising 4-1BBL. In some embodiments, the engineered erythroid cell is an enucleated cell.

In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is a NY-ESO-1/LAGE-2 antigen. In embodiments, the exogenous antigen-presenting polypeptide is an MHC Class I HLA-A2 or HLA-A24 polypeptide or single chain fusion. In embodiments, the exogenous antigen-presenting polypeptide is an MHC Class II DP4 polypeptide or single chain fusion. In some embodiments, the erythroid cell is an enucleated cell. In some embodiments, the erythroid cell is a nucleated cell.

In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is a NY-ESO-1/LAGE-2 antigen, and wherein the erythroid cell further presents, e.g. comprises on the cell surface, at least one exogenous polypeptide comprising 4-1BBL. In embodiments, the exogenous antigen-presenting polypeptide is an MHC Class I HLA-A2 or HLA-A24 polypeptide or single chain fusion. In embodiments, the exogenous antigen-presenting polypeptide is an MHC Class II DP4 polypeptide or single chain fusion. In some embodiments, the erythroid cell is an enucleated cell. In some embodiments, the erythroid cell is a nucleated cell.

In some embodiments, an aAPC of the present disclosure comprises an erythroid cell, wherein the erythroid cell presents at least one exogenous antigenic polypeptide, wherein the at least one exogenous antigenic polypeptide is a NY-ESO-1/LAGE-2 antigen as listed in Table 1. In some embodiments, an aAPC of the present disclosure comprises an erythroid cell, wherein the erythroid cell presents, e.g. comprises on the cell surface, at least one exogenous antigenic polypeptide, wherein the at least one exogenous antigenic polypeptide is a NY-ESO-1/LAGE-2 antigen as listed in Table 1, and wherein the erythroid cell further presents, e.g. comprises on the cell surface, at least one exogenous polypeptide comprising 4-1BBL. In some embodiments, an aAPC of the present disclosure comprises an erythroid cell, wherein the erythroid cell presents, e.g. comprises on the cell surface, at least one exogenous antigenic polypeptide, wherein the at least one exogenous antigenic polypeptide is a NY-ESO-1/LAGE-2 antigen selected from the NY-ESO-1/LAGE-2 antigens listed in Table 1, and further presents, e.g. comprises on the cell surface, an exogenous antigen-presenting polypeptide, wherein the exogenous antigenic polypeptide is specifically bound to the exogenous antigen-presenting polypeptide, wherein the exogenous antigen-presenting polypeptide is a MHC Class I polypeptide or single chain fusion, or a MHC Class II polypeptide or single chain fusion, of the corresponding MHC Class I/Class II HLA listed in Table 1 for the particular NY-ESO-1/LAGE-2 antigen. In some embodiments, an aAPC of the present disclosure comprises an erythroid cell, wherein the erythroid cell presents, e.g. comprises on the cell surface, at least one exogenous antigenic polypeptide, wherein the at least one exogenous antigenic polypeptide is a NY-ESO-1/LAGE-2 antigen selected from the NY-ESO-1/LAGE-2 antigens listed in Table 1, wherein the erythroid cell further presents, e.g. comprises on the cell surface, an exogenous antigen-presenting polypeptide, wherein the exogenous antigenic polypeptide is specifically bound to the exogenous antigen-presenting polypeptide, wherein the exogenous antigen-presenting polypeptide is a MHC Class I polypeptide or single chain fusion, or a MHC Class II polypeptide or single chain fusion, of the corresponding MHC Class I/Class II HLA listed in Table 1 for the particular NY-ESO-1/LAGE-2 antigen, and wherein the erythroid cell further presents, e.g. comprises on the cell surface, at least one exogenous polypeptide comprising 4-1BBL.

Also encompassed by the disclosure is an artificial antigen presenting cell (aAPC) comprising an erythroid cell, wherein the erythroid cell presents, e.g. comprises on the cell surface, at least one exogenous antigenic polypeptide comprising a NY-ESO-1/LAGE-2 derived peptide. In some embodiments, the NY-ESO-1/LAGE-2 derived peptide is an HLA class I-binding polypeptide derived from NY-ESO-1/LAGE-2. In some embodiments, the HLA class I-binding polypeptide derived from NY-ESO-1/LAGE-2 is SLLMWITQC (SEQ ID NO: 110).

In some embodiments, the erythroid cell presents, e.g. comprises on the cell surface, at least one exogenous antigenic polypeptide comprising at least one exogenous HLA class II-binding polypeptide derived from NY-ESO-1/LAGE-2. In some embodiments, the HLA class II-binding polypeptide derived from NY-ESO-1/LAGE-2 is SLLMWITQCFLPVF (SEQ ID NO: 114).

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is SLLMWITQC (SEQ ID NO: 110). In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is SLLMWITQCFLPVF (SEQ ID NO: 114).

Also encompassed by the disclosure is an artificial antigen presenting cell (aAPC) engineered to activate T cells, wherein the aAPC comprises an erythroid cell, wherein the erythroid cell presents, e.g. comprises on the cell surface, at least one exogenous antigenic polypeptide, wherein the at least one exogenous antigenic polypeptide is SLLMWITQC (SEQ ID NO: 110). In some embodiments, the disclosure features an artificial antigen presenting cell (aAPC) engineered to activate T cells, wherein the aAPC comprises an erythroid cell, wherein the erythroid cell presents, e.g. comprises on the cell surface, at least one exogenous antigenic polypeptide, wherein the at least one exogenous antigenic polypeptide is SLLMWITQCFLPVF (SEQ ID NO: 114).

Also encompassed by the disclosure is an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is SLLMWITQC (SEQ ID NO: 110). In embodiments, the exogenous antigen-presenting polypeptide is an MHC Class I HLA-A2 or HLA-A24 polypeptide or single chain fusion. In some embodiments, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class II single chain fusion, and wherein the exogenous antigenic polypeptide is SLLMWITQCFLPVF (SEQ ID NO: 114). In embodiments, the exogenous antigen-presenting polypeptide is an MHC Class II DP4 polypeptide or single chain fusion.

In one particular embodiment, an artificial antigen presenting cell comprises an erythroid cell, wherein the erythroid cell presents, e.g. comprises on the cell surface, at least one exogenous antigenic polypeptide, SLLMWITQC (SEQ ID NO: 110), fused to an exogenous antigen presenting polypeptide, MHCI HLA-A2 or HLA-24, fused to the GPA transmembrane domain (GPA).

In one particular embodiment, an artificial antigen presenting cell comprises an erythroid cell, wherein the erythroid cell presents, e.g. comprises on the cell surface, at least one exogenous antigenic polypeptide, SLLMWITQCFLPVF (SEQ ID NO: 114), fused to an exogenous antigen presenting polypeptide, MHCII HLA-DP4, fused to the GPA transmembrane domain (GPA).

In embodiments, the at least one exogenous antigenic polypeptide is a NY-ESO-1/LAGE-2 antigen selected from SLLMWITQC (SEQ ID NO: 110), MLMAQEALAFL (SEQ ID NO: 109), YLAMPFATPME (SEQ ID NO: 204), ASGPGGGAPR (SEQ ID NO: 205), LAAQERRVPR (SEQ ID NO: 111), TVSGNILTIR (SEQ ID NO: 206), APRGPHGGAASGL (SEQ ID NO: 207), MPFATPMEAEL (SEQ ID NO: 208), KEFTVSGNILTI (SEQ ID NO: 209), MPFATPMEA (SEQ ID NO: 210), FATPMEAEL (SEQ ID NO: 211), FATPMEAELAR (SEQ ID NO: 212), LAMPFATPM (SEQ ID NO: 213), ARGPESRLL (SEQ ID NO: 214), SLLMWITQCFLPVF (SEQ ID NO: 114), LLEFYLAMPFATPMEAELARRSLAQ (SEQ ID NO: 215), LLEFYLAMPFATPMEAELARRSLAQ (SEQ ID NO: 215), EFYLAMPFATPM (SEQ ID NO: 216), PGVLLKEFTVSGNILTIRLTAADHR (SEQ ID NO: 217), RLLEFYLAMPFA (SEQ ID NO: 218), QGAMLAAQERRVPRAAEVPR (SEQ ID NO: 115), PFATPMEAELARR (SEQ ID NO: 219), PGVLLKEFTVSGNILTIRLT (SEQ ID NO: 220), VLLKEFTVSG (SEQ ID NO: 221), AADHRQLQLSISSCLQQL (SEQ ID NO: 116), LLEFYLAMPFATPMEAELARRSLAQ (SEQ ID NO: 215), LKEFTVSGNILTIRL (SEQ ID NO: 222), PGVLLKEFTVSGNILTIRLTAADHR (SEQ ID NO: 217), LLEFYLAMPFATPMEAELARRSLAQ (SEQ ID NO: 215), KEFTVSGNILT (SEQ ID NO: 223), LLEFYLAMPFATPM (SEQ ID NO: 224), and AGATGGRGPRGAGA (SEQ ID NO: 119).

In some embodiments, an aAPC of the present disclosure comprises an erythroid cell, wherein the erythroid cell presents, e.g. comprises on the cell surface, at least one exogenous antigenic polypeptide, wherein the at least one exogenous antigenic polypeptide is a NY-ESO-1/LAGE-2 antigen selected from SLLMWITQC (SEQ ID NO: 110), MLMAQEALAFL (SEQ ID NO: 109), YLAMPFATPME (SEQ ID NO: 204), ASGPGGGAPR (SEQ ID NO: 205), LAAQERRVPR (SEQ ID NO: 111), TVSGNILTIR (SEQ ID NO: 206), APRGPHGGAASGL (SEQ ID NO: 207), MPFATPMEAEL (SEQ ID NO: 208), KEFTVSGNILTI (SEQ ID NO: 209), MPFATPMEA (SEQ ID NO: 210), FATPMEAEL (SEQ ID NO: 211), FATPMEAELAR (SEQ ID NO: 212), LAMPFATPM (SEQ ID NO: 213), ARGPESRLL (SEQ ID NO: 214), SLLMWITQCFLPVF (SEQ ID NO: 114), LLEFYLAMPFATPMEAELARRSLAQ (SEQ ID NO: 215), LLEFYLAMPFATPMEAELARRSLAQ (SEQ ID NO: 215), EFYLAMPFATPM (SEQ ID NO: 216), PGVLLKEFTVSGNILTIRLTAADHR (SEQ ID NO: 217), RLLEFYLAMPFA (SEQ ID NO: 218), QGAMLAAQERRVPRAAEVPR (SEQ ID NO: 115), PFATPMEAELARR (SEQ ID NO: 219), PGVLLKEFTVSGNILTIRLT (SEQ ID NO: 220), VLLKEFTVSG (SEQ ID NO: 221), AADHRQLQLSISSCLQQL (SEQ ID NO: 116), LLEFYLAMPFATPMEAELARRSLAQ (SEQ ID NO: 215), LKEFTVSGNILTIRL (SEQ ID NO: 222), PGVLLKEFTVSGNILTIRLTAADHR (SEQ ID NO: 217), LLEFYLAMPFATPMEAELARRSLAQ (SEQ ID NO: 215), KEFTVSGNILT (SEQ ID NO: 223), LLEFYLAMPFATPM (SEQ ID NO: 224), and AGATGGRGPRGAGA (SEQ ID NO: 119), wherein the erythroid cell further presents, e.g. comprises on the cell surface, at least one exogenous polypeptide comprising 4-1BBL.

In some embodiments, an aAPC of the present disclosure comprises an erythroid cell, wherein the erythroid cell presents, e.g. comprises on the cell surface, at least one exogenous antigenic polypeptide, wherein the at least one exogenous antigenic polypeptide is a NY-ESO-1/LAGE-2 antigen selected from SLLMWITQC (SEQ ID NO: 110), MLMAQEALAFL (SEQ ID NO: 109), YLAMPFATPME (SEQ ID NO: 204), ASGPGGGAPR (SEQ ID NO: 205), LAAQERRVPR (SEQ ID NO: 111), TVSGNILTIR (SEQ ID NO: 206), APRGPHGGAASGL (SEQ ID NO: 207), MPFATPMEAEL (SEQ ID NO: 208), KEFTVSGNILTI (SEQ ID NO: 209), MPFATPMEA (SEQ ID NO: 210), FATPMEAEL (SEQ ID NO: 211), FATPMEAELAR (SEQ ID NO: 212), LAMPFATPM (SEQ ID NO: 213), ARGPESRLL (SEQ ID NO: 214), SLLMWITQCFLPVF (SEQ ID NO: 114), LLEFYLAMPFATPMEAELARRSLAQ (SEQ ID NO: 215), LLEFYLAMPFATPMEAELARRSLAQ (SEQ ID NO: 215), EFYLAMPFATPM (SEQ ID NO: 216), PGVLLKEFTVSGNILTIRLTAADHR (SEQ ID NO: 217), RLLEFYLAMPFA (SEQ ID NO: 218), QGAMLAAQERRVPRAAEVPR (SEQ ID NO: 115), PFATPMEAELARR (SEQ ID NO: 219), PGVLLKEFTVSGNILTIRLT (SEQ ID NO: 220), VLLKEFTVSG (SEQ ID NO: 221), AADHRQLQLSISSCLQQL (SEQ ID NO: 116), LLEFYLAMPFATPMEAELARRSLAQ (SEQ ID NO: 215), LKEFTVSGNILTIRL (SEQ ID NO: 222), PGVLLKEFTVSGNILTIRLTAADHR (SEQ ID NO: 217), LLEFYLAMPFATPMEAELARRSLAQ (SEQ ID NO: 215), KEFTVSGNILT (SEQ ID NO: 223), LLEFYLAMPFATPM (SEQ ID NO: 224), and AGATGGRGPRGAGA (SEQ ID NO: 119), wherein the erythroid cell further presents, e.g. comprises on the cell surface, an exogenous antigen-presenting polypeptide, wherein the exogenous antigenic polypeptide is specifically bound to the exogenous antigen-presenting polypeptide, wherein the exogenous antigen-presenting polypeptide is a MHC Class I polypeptide or single chain fusion, or a MHC Class II polypeptide or single chain fusion, of the corresponding MHC Class I/Class II HLA listed in Table 1 for the particular NY-ESO-1/LAGE-2 antigen, and wherein the erythroid cell further presents, e.g. comprises on the cell surface, at least one exogenous polypeptide comprising 4-1BBL.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is SLLMWITQC (SEQ ID NO: 110), and wherein the erythroid cell further presents, e.g. comprises on the cell surface, an exogenous polypeptide comprising 4-1BBL.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is SLLMWITQCFLPVF (SEQ ID NO: 114), and wherein the erythroid cell further presents, e.g. comprises on the cell surface, an exogenous polypeptide comprising 4-1BBL.

An aAPC as described herein, comprising (e.g. comprising on the cell surface), an exogenous antigenic polypeptide comprising a NY-ESO-1/LAGE-2 antigen, can be used in the treatment of cancer, as described in more detail below. An aAPC as described herein, comprising (e.g. comprising on the cell surface) an exogenous antigenic polypeptide comprising a NY-ESO-1/LAGE-2 antigen, and further comprising (e.g. comprising on the cell surface) an exogenous polypeptide comprising 4-1BBL, can be used in the treatment of cancer, as described in more detail below. An aAPC as described herein, comprising (e.g. comprising on the cell surface) at least one exogenous NY-ESO-1/LAGE-2 derived peptide (e.ge.g., SLLMWITQC (SEQ ID NO: 110) or SLLMWITQCFLPVF (SEQ ID NO: 114)) as described herein, can be used in the treatment of cancer, as described in more detail below. An aAPC as described herein, comprising (e.g. comprising on the cell surface) at least one exogenous NY-ESO-1/LAGE-2 derived peptide (e.g., SLLMWITQC (SEQ ID NO: 110) or SLLMWITQCFLPVF (SEQ ID NO: 114)), and further comprising an exogenous polypeptide comprising 4-1BBL, as described herein, can be used in the treatment of cancer, as described in more detail below.

Telomerase/hTERT

Telomerase reverse transcriptase (abbreviated to TERT, or hTERT in humans) is a ribonucleoprotein enzyme essential for the replication of chromosome termini in most eukaryotes. Telomerase maintains telomere ends by addition of the telomere repeat TTAGGG. Telomerase expression plays a role in cellular senescence, as it is normally repressed in postnatal somatic cells, resulting in progressive shortening of telomeres. Telomerase activity is associated with the number of times a cell can divide playing an important role in the immortality of cell lines, such as cancer cells.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a telomerase antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a telomerase antigen, and wherein the erythroid cell further presents, e.g. comprises on the cell surface, an exogenous polypeptide comprising 4-1BBL. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a human telomerase (hTERT) antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a human telomerase (hTERT) antigen, and wherein the erythroid cell further presents, e.g. comprises on the cell surface, an exogenous polypeptide comprising 4-1BBL. In some embodiments, the erythroid cell is an enucleated cell. In some embodiments, the erythroid cell is a nucleated cell.

In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is a telomerase antigen. In some embodiments, the telomerase antigen is human telomerase (hTERT) antigen. In some embodiments, the erythroid cell is an enucleated cell. In some embodiments, the erythroid cell is a nucleated cell.

In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is a telomerase antigen, and wherein the erythroid cell further presents, e.g. comprises on the cell surface, an exogenous polypeptide comprising 4-1BBL. In some embodiments, the telomerase antigen is human telomerase (hTERT) antigen. In some embodiments, the erythroid cell is an enucleated cell. In some embodiments, the erythroid cell is a nucleated cell.

Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ILAKFLHWL (SEQ ID NO: 658). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RLVDDFLLV (SEQ ID NO: 659). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RPGLLGASVLGLDDI (SEQ ID NO: 663). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is LTDLQPYMRQFVAHL (SEQ ID NO: 664). Also encompassed by the disclosure is an artificial antigen presenting cell (aAPC) engineered to activate T cells, wherein the aAPC comprises an erythroid cell, wherein the erythroid cell presents, e.g. comprises on the cell surface, at least one exogenous antigenic polypeptide, wherein the at least one exogenous antigenic polypeptide is ILAKFLHWL (SEQ ID NO: 658). Also encompassed by the disclosure is an artificial antigen presenting cell (aAPC) engineered to activate T cells, wherein the aAPC comprises an erythroid cell, wherein the erythroid cell presents, e.g. comprises on the cell surface, at least one exogenous antigenic polypeptide, wherein the at least one exogenous antigenic polypeptide is RLVDDFLLV (SEQ ID NO: 659). Also encompassed by the disclosure is an artificial antigen presenting cell (aAPC) engineered to activate T cells, wherein the aAPC comprises an erythroid cell, wherein the erythroid cell presents, e.g. comprises on the cell surface, at least one exogenous antigenic polypeptide, wherein the at least one exogenous antigenic polypeptide is RPGLLGASVLGLDDI (SEQ ID NO: 663). Also encompassed by the disclosure is an artificial antigen presenting cell (aAPC) engineered to activate T cells, wherein the aAPC comprises an erythroid cell, wherein the erythroid cell presents, e.g. comprises on the cell surface, at least one exogenous antigenic polypeptide, wherein the at least one exogenous antigenic polypeptide is LTDLQPYMRQFVAHL (SEQ ID NO: 664). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ILAKFLHWL (SEQ ID NO: 658), and wherein the erythroid cell further presents, e.g. comprises on the cell surface, an exogenous polypeptide comprising 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RLVDDFLLV SEQ ID NO: 659), and wherein the erythroid cell further presents, e.g. comprises on the cell surface, an exogenous polypeptide comprising 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RPGLLGASVLGLDDI (SEQ ID NO: 663), and wherein the erythroid cell further presents, e.g. comprises on the cell surface, an exogenous polypeptide comprising 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is LTDLQPYMRQFVAHL (SEQ ID NO: 664), and wherein the erythroid cell further presents, e.g. comprises on the cell surface, an exogenous polypeptide comprising 4-1BBL. Also encompassed by the disclosure is an artificial antigen presenting cell (aAPC) engineered to activate T cells, wherein the aAPC comprises an erythroid cell, wherein the erythroid cell presents, e.g. comprises on the cell surface, at least one exogenous antigenic polypeptide, wherein the at least one exogenous antigenic polypeptide is ILAKFLHWL (SEQ ID NO: 658), and wherein the erythroid cell further presents, e.g. comprises on the cell surface, an exogenous polypeptide comprising 4-1BBL. Also encompassed by the disclosure is an artificial antigen presenting cell (aAPC) engineered to activate T cells, wherein the aAPC comprises an erythroid cell, wherein the erythroid cell presents, e.g. comprises on the cell surface, at least one exogenous antigenic polypeptide, wherein the at least one exogenous antigenic polypeptide is RLVDDFLLV (SEQ ID NO: 659), and wherein the erythroid cell further presents, e.g. comprises on the cell surface, an exogenous polypeptide comprising 4-1BBL. Also encompassed by the disclosure is an artificial antigen presenting cell (aAPC) engineered to activate T cells, wherein the aAPC comprises an erythroid cell, wherein the erythroid cell presents, e.g. comprises on the cell surface, at least one exogenous antigenic polypeptide, wherein the at least one exogenous antigenic polypeptide is RPGLLGASVLGLDDI (SEQ ID NO: 663), and wherein the erythroid cell further presents, e.g. comprises on the cell surface, an exogenous polypeptide comprising 4-1BBL. Also encompassed by the disclosure is an artificial antigen presenting cell (aAPC) engineered to activate T cells, wherein the aAPC comprises an erythroid cell, wherein the erythroid cell presents, e.g. comprises on the cell surface, at least one exogenous antigenic polypeptide, wherein the at least one exogenous antigenic polypeptide is LTDLQPYMRQFVAHL (SEQ ID NO: 664), and wherein the erythroid cell further presents, e.g. comprises on the cell surface, an exogenous polypeptide comprising 4-1BBL.

Also encompassed by the disclosure is an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is ILAKFLHWL (SEQ ID NO: 658). In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion.

Also encompassed by the disclosure is an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is ILAKFLHWL (SEQ ID NO: 658), and wherein the erythroid cell further presents, e.g. comprises on the cell surface, an exogenous polypeptide comprising 4-1BBL. In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion.

Also encompassed by the disclosure is an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is RLVDDFLLV (SEQ ID NO: 659). In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion.

Also encompassed by the disclosure is an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is RLVDDFLLV (SEQ ID NO: 659), and wherein the erythroid cell further presents, e.g. comprises on the cell surface, an exogenous polypeptide comprising 4-1BBL. In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion.

Also encompassed by the disclosure is an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class II polypeptide or single chain fusion, and wherein the exogenous antigenic polypeptide is RPGLLGASVLGLDDI (SEQ ID NO: 663). In embodiments, the exogenous antigen-presenting polypeptide is an MHC Class II HLA-DR7 polypeptide or single chain fusion. In some embodiments, the exogenous antigen-presenting polypeptide is an MHC Class II HLA-DR7 single chain fusion.

Also encompassed by the disclosure is an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class II polypeptide or single chain fusion, and wherein the exogenous antigenic polypeptide is RPGLLGASVLGLDDI (SEQ ID NO: 663), and wherein the erythroid cell further presents, e.g. comprises on the cell surface, an exogenous polypeptide comprising 4-1BBL. In embodiments, the exogenous antigen-presenting polypeptide is an MHC Class II HLA-DR7 polypeptide or single chain fusion. In some embodiments, the exogenous antigen-presenting polypeptide is an MHC Class II HLA-DR7 single chain fusion.

Also encompassed by the disclosure is an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class II polypeptide or single chain fusion, and wherein the exogenous antigenic polypeptide is LTDLQPYMRQFVAHL (SEQ ID NO: 664). In embodiments, the exogenous antigen-presenting polypeptide is an MHC Class II HLA-DR11 polypeptide or single chain fusion. In some embodiments, the exogenous antigen-presenting polypeptide is an MHC Class II HLA-DR11 single chain fusion.

Also encompassed by the disclosure is an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class II polypeptide or single chain fusion, and wherein the exogenous antigenic polypeptide is LTDLQPYMRQFVAHL (SEQ ID NO: 664), and wherein the erythroid cell further presents, e.g. comprises on the cell surface, an exogenous polypeptide comprising 4-1BBL. In embodiments, the exogenous antigen-presenting polypeptide is an MHC Class II HLA-DR11 polypeptide or single chain fusion. In some embodiments, the exogenous antigen-presenting polypeptide is an MHC Class II HLA-DR11 single chain fusion.

In one particular embodiment, an artificial antigen presenting cell comprises an erythroid cell, wherein the erythroid cell presents, e.g. comprises on the cell surface, at least one exogenous antigenic polypeptide, ILAKFLHWL (SEQ ID NO: 658), fused to an exogenous antigen presenting polypeptide, MHCI HLA-A2, fused to the GPA transmembrane domain (GPA).

In one particular embodiment, an artificial antigen presenting cell comprises an erythroid cell, wherein the erythroid cell presents, e.g. comprises on the cell surface, at least one exogenous antigenic polypeptide, ILAKFLHWL (SEQ ID NO: 658), fused to an exogenous antigen presenting polypeptide, MHCI HLA-A2, fused to the GPA transmembrane domain (GPA), and wherein the erythroid cell further presents, e.g. comprises on the cell surface, an exogenous polypeptide comprising 4-1BBL.

An aAPC as described herein, presenting (e.g. comprising on the cell surface) a telomerase antigen, in particular a hTERT antigen, can be used in the treatment of cancer, as described in more detail below. An aAPC as described herein, presenting (e.g. comprising on the cell surface) a telomerase antigen, in particular a hTERT antigen, and further presenting (e.g. comprising on the cell surface) an exogenous polypeptide comprising 4-1BBL, can be used in the treatment of cancer, as described in more detail below. An aAPC as described herein, presenting (e.g. comprising on the cell surface)_a hTERT antigen described above, can be used in the treatment of cancer, as described in more detail below. An aAPC as described herein, presenting (e.g. comprising on the cell surface) a hTERT antigen described above, and further presenting (e.g. comprising on the cell surface) an exogenous polypeptide comprising 4-1BBL, can be used in the treatment of cancer, as described in more detail below.

### Myelin Oligodendrocyte Glycoprotein (MOG)

Myelin Oligodendrocyte Glycoprotein (MOG) is a membrane protein expressed on the oligodendrocyte cell surface and the outermost surface of myelin sheaths. Due to this localization, MOG is a primary target antigen involved in immune-mediated demyelination. MOG protein may be involved in completion and maintenance of the myelin sheath and in cell-cell communication.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MOG antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MOG antigen, and wherein the erythroid cell further presents, e.g. comprises on the cell surface, an exogenous polypeptide comprising a coinhibitory polypeptide. In some embodiments, the coinhibitory polypeptide is PD-L1. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a MOG antigen, and wherein the erythroid cell further presents, e.g. comprises on the cell surface, an exogenous polypeptide comprising a Treg expansion polypeptide. In some embodiments, the Treg expansion polypeptide is IL-2. In some embodiments, the Treg expansion polypeptide is CD25-specific IL-2. In some embodiments, the MOG antigen is human MOG antigen. In some embodiments, the erythroid cell is an enucleated cell. In some embodiments, the erythroid cell is a nucleated cell.

In another aspect, the disclosure features an artificial antigen presenting cell (aAPC) engineered to inhibit T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is a MOG antigen. In another aspect, the disclosure features an artificial antigen presenting cell (aAPC) engineered to inhibit T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is a MOG antigen, and wherein the erythroid cell further presents, e.g. comprises on the cell surface, an exogenous polypeptide comprising a coinhibitory polypeptide. In some embodiments, the coinhibitory polypeptide is PD-L1. In another aspect, the disclosure features an artificial antigen presenting cell (aAPC) engineered to activate regulatory T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is a MOG antigen, and wherein the erythroid cell further presents, e.g. comprises on the cell surface, an exogenous polypeptide comprising a Treg expansion polypeptide. In some embodiments, the Treg expansion polypeptide is CD25-specific IL-2. In some embodiments, the MOG antigen is human MOG antigen. In some embodiments, the erythroid cell is an enucleated cell. In some embodiments, the erythroid cell is a nucleated cell.

Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is MEVGWYRSPFSRVVHLYRNGK (SEQ ID NO: 690).

Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is MEVGWYRSPFSRVVHLYRNGK (SEQ ID NO: 690), and wherein the erythroid cell further presents, e.g. comprises on the cell surface, an exogenous polypeptide comprising a coinhibitory polypeptide. In some embodiments, the coinhibitory polypeptide is PD-L1.

Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is MEVGWYRSPFSRVVHLYRNGK (SEQ ID NO: 690), and wherein the erythroid cell further presents, e.g. comprises on the cell surface, an exogenous polypeptide comprising a Treg expansion polypeptide. In some embodiments, the Treg expansion polypeptide is CD25-specific IL-2.

Also encompassed by the disclosure is an artificial antigen presenting cell (aAPC) engineered to activate regulatory T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class II polypeptide or single chain fusion, and wherein the exogenous antigenic polypeptide is MEVGWYRSPFSRVVHLYRNGK (SEQ ID NO: 690).

Also encompassed by the disclosure is an artificial antigen presenting cell (aAPC) engineered to inhibit T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class II polypeptide or single chain fusion, and wherein the exogenous antigenic polypeptide is MEVGWYRSPFSRVVHLYRNGK (SEQ ID NO: 690), and wherein the erythroid cell further presents, e.g. comprises on the cell surface, an exogenous polypeptide comprising a coinhibitory polypeptide. In some embodiments, the coinhibitory polypeptide is PD-L1

In one particular embodiment, an artificial antigen presenting cell comprises an erythroid cell, wherein the erythroid cell presents, e.g. comprises on the cell surface, at least one exogenous antigenic polypeptide, wherein the exogenous antigenic polypeptide is a MOG antigen, fused to an exogenous antigen presenting polypeptide, MHCII, fused to the GPA transmembrane domain (GPA). In some embodiments, the MOG antigen is human MOG antigen. In some embodiments, the MOG antigen is fused to an exogenous antigen presenting polypeptide, MHCII, fused to GPA as a single chain fusion.

In one particular embodiment, an artificial antigen presenting cell comprises an erythroid cell, wherein the erythroid cell presents, e.g. comprises on the cell surface, at least one exogenous antigenic polypeptide, wherein the exogenous antigenic polypeptide is a MOG antigen, fused to an exogenous antigen presenting polypeptide, MHCII, fused to the GPA transmembrane domain (GPA), and wherein the erythroid cell further presents, e.g. comprises on the cell surface, an exogenous polypeptide comprising a coinhibitory polypeptide. In some embodiments, the coinhibitory polypeptide is PD-L1. In some embodiments, the MOG antigen is human MOG antigen. In some embodiments, the MOG antigen is fused to an exogenous antigen presenting polypeptide, MHCII, fused to GPA as a single chain fusion.

In one particular embodiment, an artificial antigen presenting cell comprises an erythroid cell, wherein the erythroid cell presents, e.g. comprises on the cell surface, at least one exogenous antigenic polypeptide, wherein the exogenous antigenic polypeptide is a MOG antigen, fused to an exogenous antigen presenting polypeptide, MHCII, fused to the GPA transmembrane domain (GPA), and wherein the erythroid cell further presents, e.g. comprises on the cell surface, an exogenous polypeptide comprising a Treg expansion polypeptide. In some embodiments, the Treg expansion polypeptide is IL-2. In some embodiments, the Treg expansion polypeptide is CD25-specific IL-2. In some embodiments, the MOG antigen is human MOG antigen. In some embodiments, the MOG antigen is fused to an exogenous antigen presenting polypeptide, MHCII, fused to GPA as a single chain fusion.

An aAPC as described herein, presenting (e.g. comprising on the cell surface) a MOG antigen, can be used in the treatment of multiple sclerosis, as described in more detail below. An aAPC as described herein, presenting (e.g. comprising on the cell surface) a MOG antigen, and further presenting (e.g. comprising on the cell surface) an exogenous polypeptide comprising a coinhibitory polypeptide, can be used in the treatment of multiple sclerosis, as described in more detail below. An aAPC as described herein, presenting (e.g. comprising on the cell surface) a MOG antigen, and further presenting (e.g. comprising on the cell surface) an exogenous polypeptide comprising a coinhibitory polypeptide, wherein the coinhibitory polypeptide is PD-L1, can be used in the treatment of multiple sclerosis, as described in more detail below. An aAPC as described herein, presenting (e.g. comprising on the cell surface) a MOG antigen, and further presenting (e.g. comprising on the cell surface) an exogenous polypeptide comprising a Treg expansion polypeptide, can be used in the treatment of multiple sclerosis, as described in more detail below. An aAPC as described herein, presenting (e.g. comprising on the cell surface) a MOG antigen, and further presenting (e.g. comprising on the cell surface) an exogenous polypeptide comprising a Treg expansion polypeptide, wherein the Treg expansion polypeptide is CD25-specific IL-2, can be used in the treatment of multiple sclerosis, as described in more detail below.

gp100

Glycoprotein 100, gp100 or Melanocyte protein PMEL is a type I transmembrane glycoprotein enriched in melanosomes.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a gp100 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is a gp100 antigen, and wherein the erythroid cell further presents, e.g. comprises on the cell surface, an exogenous polypeptide comprising a costimulatory polypeptide. In some embodiments, the costimulatory polypeptide is 4-1BBL. In some embodiments, the gp100 antigen is human gp100 antigen. In some embodiments, the erythroid cell is an enucleated cell. In some embodiments, the erythroid cell is a nucleated cell.

In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is a gp100 antigen. In some embodiments, the exogenous antigen-presenting polypeptide is MHCI. In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is a gp100 antigen, and wherein the erythroid cell further presents, e.g. comprises on the cell surface, an exogenous polypeptide comprising a costimulatory polypeptide. In some embodiments, the costimulatory polypeptide is 4-1BBL. In some embodiments, the exogenous antigen-presenting polypeptide is MHCI. In some embodiments, the gp100 antigen is human gp100 antigen. In some embodiments, the erythroid cell is an enucleated cell. In some embodiments, the erythroid cell is a nucleated cell.

Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RLMKQDFSV (SEQ ID NO: 314). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RLPRIFCSC (SEQ ID NO: 315). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is LIYRRRLMK (SEQ ID NO: 316). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ALLAVGATK (SEQ ID NO: 317). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is IALNFPGSQK (SEQ ID NO: 318). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RSYVPLAHR (SEQ ID NO: 319). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ALNFPGSQK (SEQ ID NO: 320). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ALNFPGSQK (SEQ ID NO: 320). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is VYFFLPDHL (SEQ ID NO: 321). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RTKQLYPEW (SEQ ID NO: 322). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is HTMEVTVYHR (SEQ ID NO: 323). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is SSPGCQPPA (SEQ ID NO: 324). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is VPLDCVLYRY (SEQ ID NO: 325). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is LPHSSSHWL (SEQ ID NO: 326). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is SNDGPTLI (SEQ ID NO: 327). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is GRAMLGTHTMEVTVY (SEQ ID NO: 328). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is WNRQLYPEWTEAQRLD (SEQ ID NO: 329). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is TTEWVETTARELPIPEPE (SEQ ID NO: 330). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is TGRAMLGTHTMEVTVYH (SEQ ID NO: 331). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is GRAMLGTHTMEVTVY (SEQ ID NO: 328).

Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RLMKQDFSV (SEQ ID NO: 314), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RLPRIFCSC (SEQ ID NO: 315), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is LIYRRRLMK (SEQ ID NO: 316), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ALLAVGATK (SEQ ID NO: 317), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is IALNFPGSQK (SEQ ID NO: 318), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RSYVPLAHR (SEQ ID NO: 319), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ALNFPGSQK (SEQ ID NO: 320), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ALNFPGSQK (SEQ ID NO: 320), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is VYFFLPDHL (SEQ ID NO: 321), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RTKQLYPEW (SEQ ID NO: 322), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is HTMEVTVYHR (SEQ ID NO: 323), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is SSPGCQPPA (SEQ ID NO: 324), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is VPLDCVLYRY (SEQ ID NO: 325), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is LPHSSSHWL (SEQ ID NO: 326), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is SNDGPTLI (SEQ ID NO: 327), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is GRAMLGTHTMEVTVY (SEQ ID NO: 328), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is WNRQLYPEWTEAQRLD (SEQ ID NO: 329), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is TTEWVETTARELPIPEPE (SEQ ID NO: 330), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is TGRAMLGTHTMEVTVYH (SEQ ID NO: 331), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is GRAMLGTHTMEVTVY (SEQ ID NO: 328), wherein the erythroid cell further presents, e.g. comprises on the cell surface, an exogenous polypeptide comprising a costimulatory polypeptide. In some embodiments, the costimulatory polypeptide is 4-1BBL.

Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RLMKQDFSV (SEQ ID NO: 314), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RLPRIFCSC (SEQ ID NO: 315), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is LIYRRRLMK (SEQ ID NO: 316), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ALLAVGATK (SEQ ID NO: 317), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is IALNFPGSQK (SEQ ID NO: 318), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RSYVPLAHR (SEQ ID NO: 319), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ALNFPGSQK (SEQ ID NO: 320), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ALNFPGSQK (SEQ ID NO: 320), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is VYFFLPDHL (SEQ ID NO: 321), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RTKQLYPEW (SEQ ID NO: 322), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is HTMEVTVYHR (SEQ ID NO: 323), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is SSPGCQPPA (SEQ ID NO: 324), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is VPLDCVLYRY (SEQ ID NO: 325), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is LPHSSSHWL (SEQ ID NO: 326), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is SNDGPTLI (SEQ ID NO: 327), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is GRAMLGTHTMEVTVY (SEQ ID NO: 328), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is WNRQLYPEWTEAQRLD (SEQ ID NO: 329), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is TTEWVETTARELPIPEPE (SEQ ID NO: 330), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is TGRAMLGTHTMEVTVYH (SEQ ID NO: 331), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is GRAMLGTHTMEVTVY (SEQ ID NO: 328), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion.

Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RLMKQDFSV (SEQ ID NO: 314), and an exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RLPRIFCSC (SEQ ID NO: 315), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is LIYRRRLMK (SEQ ID NO: 316), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ALLAVGATK (SEQ ID NO: 317), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is IALNFPGSQK (SEQ ID NO: 318), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RSYVPLAHR (SEQ ID NO: 319), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ALNFPGSQK (SEQ ID NO: 320), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is ALNFPGSQK (SEQ ID NO: 320), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is VYFFLPDHL (SEQ ID NO: 321), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RTKQLYPEW (SEQ ID NO: 322), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is HTMEVTVYHR (SEQ ID NO: 323), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is SSPGCQPPA (SEQ ID NO: 324), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is VPLDCVLYRY (SEQ ID NO: 325), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is LPHSSSHWL (SEQ ID NO: 326), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is SNDGPTLI (SEQ ID NO: 327), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is GRAMLGTHTMEVTVY (SEQ ID NO: 328), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is WNRQLYPEWTEAQRLD (SEQ ID NO: 329), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is TTEWVETTARELPIPEPE (SEQ ID NO: 330), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is TGRAMLGTHTMEVTVYH (SEQ ID NO: 331), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is GRAMLGTHTMEVTVY (SEQ ID NO: 328), and an exogenous antigen-presenting polypeptide, wherein the exogenous antigenic polypeptide is specifically bound to the exogenous antigen-presenting polypeptide, wherein the exogenous antigen-presenting polypeptide is an MHC class I polypeptide or single chain fusion, wherein the erythroid cell further presents, e.g. comprises on the cell surface, an exogenous polypeptide comprising a costimulatory polypeptide. In some embodiments, the costimulatory polypeptide is 4-1BBL.

An aAPC as described herein, presenting, e.g. comprising on the cell surface, a gp100 antigen, can be used in the treatment of cancer, as described in more detail below. An aAPC as described herein, presenting, e.g. comprising on the cell surface, a gp100 antigen, and further presenting, e.g. comprising on the cell surface, an exogenous polypeptide comprising a costimulatory polypeptide, can be used in the treatment of cancer, as described in more detail below. An aAPC as described herein, presenting, e.g. comprising on the cell surface, a gp100 antigen, and further presenting, e.g. comprising on the cell surface, an exogenous polypeptide comprising a costimulatory polypeptide, wherein the costimulatory polypeptide is 4-1BBL, can be used in the treatment of cancer, as described in more detail below. An aAPC as described herein, presenting, e.g. comprising on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is a gp100 antigen, can be used in the treatment of cancer, as described in more detail below. An aAPC as described herein, presenting, e.g. comprising on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is a gp100 antigen, and further presenting, e.g. comprising on the cell surface, an exogenous polypeptide comprising a costimulatory polypeptide, wherein the costimulatory polypeptide is 4-1BBL, can be used in the treatment of cancer, as described in more detail below. In certain embodiments, the cancer is melanoma.

### Epstein Barr Virus (EBV)

EBV is a human gamma herpesvirus with a tropism for B lymphocytes (Kieff and Liebowitz, in Virology, eds. Fields, B. N., Knipe, D. M. et al., p. 1889-1919, Raven Press, Ltd.: New York, 1990). EBV is an extremely common environmental agent infecting 80-100 percent of the individuals around the world. The initial or primary infection may be acute or sub-clinical. This is followed by a long period during which the EBV infection is latent in B lymphocytes present in the circulating blood, lymph nodes, and spleen.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is an EBV antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is an EBV antigen, and wherein the erythroid cell further presents, e.g. comprises on the cell surface, an exogenous polypeptide comprising a costimulatory polypeptide. In some embodiments, the costimulatory polypeptide is 4-1BBL. In some embodiments, the EBV antigen is human EBV antigen. In some embodiments, the erythroid cell is an enucleated cell. In some embodiments, the erythroid cell is a nucleated cell.

In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is an EBV antigen. In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is an EBV antigen, and wherein the erythroid cell further presents, e.g. comprises on the cell surface, an exogenous polypeptide comprising a costimulatory polypeptide. In some embodiments, the costimulatory polypeptide is 4-1BBL. In some embodiments, the EBV antigen is human EBV antigen. In some embodiments, the erythroid cell is an enucleated cell. In some embodiments, the erythroid cell is a nucleated cell.

Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is a gp350 antigenic peptide. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is VLQWASLAV (SEQ ID NO: 698). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is an EBNA1 antigenic polypeptide. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is FMVFLQTHI (SEQ ID NO: 699). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is FLQTHIFAEV (SEQ ID NO: 700). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is SIVCYFMVFL (SEQ ID NO: 701). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is CLGGLLTMV (SEQ ID NO: 691). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is GLCTLVAML (SEQ ID NO: 692). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is FLYALALLL (SEQ ID NO: 693). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is YVLDHLIVV (SEQ ID NO: 694). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RLRAEAQVK (SEQ ID NO: 695). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is AVFDRKSDAK (SEQ ID NO: 696). Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RPPIFIRLL (SEQ ID NO: 697).

Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is a gp350 antigenic polypeptide, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is VLQWASLAV (SEQ ID NO: 698), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is an EBNA1 antigenic polypeptide, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is FMVFLQTHI (SEQ ID NO: 699), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is FLQTHIFAEV (SEQ ID NO: 700), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is SIVCYFMVFL (SEQ ID NO: 701), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is CLGGLLTMV (SEQ ID NO: 691), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is GLCTLVAML (SEQ ID NO: 692), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is FLYALALLL (SEQ ID NO: 693), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is YVLDHLIVV (SEQ ID NO: 694), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RLRAEAQVK (SEQ ID NO: 695), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is AVFDRKSDAK (SEQ ID NO: 696), wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is RPPIFIRLL (SEQ ID NO: 697), wherein the erythroid cell further presents, e.g. comprises on the cell surface, an exogenous polypeptide comprising a costimulatory polypeptide. In some embodiments, the costimulatory polypeptide is 4-1BBL.

Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigen-presenting polypeptide is an MHC class I polypeptide or single chain fusion, and wherein the exogenous antigenic polypeptide is an gp350 antigenic polypeptide. In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion. Also encompassed by the disclosure is an artificial antigen presenting cell (aAPC) comprising an erythroid cell, wherein the erythroid cell presents an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is VLQWASLAV (SEQ ID NO: 698). In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion. Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigen-presenting polypeptide is an MHC class I polypeptide or single chain fusion, and wherein the exogenous antigenic polypeptide is an EBNA1 antigenic polypeptide. In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion. Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is FMVFLQTHI (SEQ ID NO: 699). In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion. Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is FLQTHIFAEV (SEQ ID NO: 700). In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion. Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is SIVCYFMVFL (SEQ ID NO: 701). In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion. Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigen-presenting polypeptide is an MHC class I polypeptide or single chain fusion, and wherein the exogenous antigenic polypeptide is CLGGLLTMV (SEQ ID NO: 691). In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion. Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is GLCTLVAML (SEQ ID NO: 692). In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion. Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is FLYALALLL (SEQ ID NO: 693). In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion. Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is YVLDHLIVV (SEQ ID NO: 694). In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion. Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is RLRAEAQVK (SEQ ID NO: 695). In embodiments, the exogenous antigen-presenting polypeptide is an MHC Class I HLA-A3 polypeptide or single chain fusion. In some embodiments, the exogenous antigen-presenting polypeptide is an MHC Class I HLA-A3 single chain fusion. Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is AVFDRKSDAK (SEQ ID NO: 696). In embodiments, the exogenous antigen-presenting polypeptide is an MHC Class I HLA-A11 polypeptide or single chain fusion. In some embodiments, the exogenous antigen-presenting polypeptide is an MHC Class I HLA-A11 single chain fusion. Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is RPPIFIRLL (SEQ ID NO: 697). In embodiments, the exogenous antigen-presenting polypeptide is an MHC Class I HLA-B7 polypeptide or single chain fusion. In some embodiments, the exogenous antigen-presenting polypeptide is an MHC Class I HLA-B7 single chain fusion.

Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is a gp350 antigenic peptide, e.g., VLQWASLAV (SEQ ID NO: 698), wherein the erythroid cell further presents, e.g. comprises on the cell surface, an exogenous polypeptide comprising a costimulatory polypeptide. In some embodiments, the costimulatory polypeptide is 4-1BBL. In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion.

Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigen-presenting polypeptide is an MHC class I polypeptide or single chain fusion, and wherein the exogenous antigenic polypeptide is an EBNA1 antigenic peptide, e.g., FMVFLQTHI (SEQ ID NO: 699), FLQTHIFAEV (SEQ ID NO: 700), SIVCYFMVFL (SEQ ID NO: 701) or one of the EBV antigenic polypeptide listed in Table 1, wherein the erythroid cell further presents, e.g. comprises on the cell surface, an exogenous polypeptide comprising a costimulatory polypeptide. In some embodiments, the costimulatory polypeptide is 4-1BBL. In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion.

An aAPC as described herein, presenting, e.g. comprising on the cell surface, an EBV antigen, can be used in the treatment of an autoimmune disease associated with an infectious agent. In certain embodiments, the exogenous antigenic polypeptides are presented on antigen-presenting polypeptides, e.g., the exogenous antigenic polypeptide is specifically bound to the exogenous antigen-presenting polypeptides, e.g. histocompatibility molecules (MHCI, MHCII). In some embodiments, the autoimmune disease associated with an infectious agent is multiple sclerosis (MS) as described in more detail below. An aAPC as described herein, presenting, e.g. comprising on the cell surface, an EBV antigen, and further presenting, e.g. comprising on the cell surface, an exogenous polypeptide comprising a costimulatory polypeptide, can be used in the treatment an autoimmune disease associated with an infectious agent. In some embodiments, the autoimmune disease associated with an infectious agent is multiple sclerosis (MS) as described in more detail below. An aAPC as described herein, presenting, e.g. comprising on the cell surface, an EBV antigen, and further presenting, e.g. comprising on the cell surface, an exogenous polypeptide comprising a costimulatory polypeptide, wherein the costimulatory polypeptide is 4-1BBL, can be used in the treatment of an autoimmune disease associated with an infectious agent. In some embodiments, the autoimmune disease associated with an infectious agent is multiple sclerosis (MS) as described in more detail below.

### Human Papilloma Virus (HPV)

Papillomaviruses are small DNA tumour viruses, which are highly species specific. So far, over 70 individual human papillomavirus (HPV) genotypes have been described. HPVs are generally specific either for the skin (e.g. HPV-1 and -2) or mucosal surfaces (e.g. HPV-6 and -11) and usually cause benign tumors (warts) that persist for several months or years. Some HPVs are also associated with cancers, such as HPV-positive head and neck and cervical cancers. The strongest positive association between an HPV and human cancer is that which exists between HPV-16 and HPV-18 and cervical carcinoma.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is an HPV antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is an HPV-E7 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is an HPV-E6 antigen. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is an HPV antigen, and wherein the erythroid cell further presents, e.g. comprises on the cell surface, an exogenous polypeptide comprising a costimulatory polypeptide. In some embodiments, the costimulatory polypeptide is 4-1BBL. In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is an HPV-E7 antigen, and wherein the erythroid cell further presents, e.g. comprises on the cell surface, an exogenous polypeptide comprising a costimulatory polypeptide. In some embodiments, the costimulatory polypeptide is 4-1BBL. In some embodiments, the HPV antigen is human HPV antigen. In some embodiments, the erythroid cell is an enucleated cell. In some embodiments, the erythroid cell is a nucleated cell.

In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is an HPV antigen. In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is an HPV-E7 antigen. In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is an HPV antigen, and wherein the erythroid cell further presents, e.g. comprises on the cell surface, an exogenous polypeptide comprising a costimulatory polypeptide. In another aspect, the disclosure features an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, wherein the exogenous antigenic polypeptide is an HPV-E7 antigen, and wherein the erythroid cell further presents, e.g. comprises on the cell surface, an exogenous polypeptide comprising a costimulatory polypeptide. In some embodiments, the costimulatory polypeptide is 4-1BBL. In some embodiments, the HPV antigen is human HPV antigen. In some embodiments, the erythroid cell is an enucleated cell. In some embodiments, the erythroid cell is a nucleated cell.

Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is YMLDLQPET (SEQ ID NO: 713), YMLDLQPETT (SEQ ID NO: 714), or TIHDIILECV (SEQ ID NO: 712). In some embodiments, the exogenous antigenic polypeptide is YMLDLQPET (SEQ ID NO: 713).

Also encompassed by the disclosure is an artificial antigen presenting cell (aAPC) comprising an erythroid cell, wherein the erythroid cell presents, e.g. comprises on the cell surface, at least one exogenous antigenic polypeptide, wherein the exogenous antigenic polypeptide is YMLDLQPET (SEQ ID NO: 713), YMLDLQPETT (SEQ ID NO: 714), or TIHDIILECV (SEQ ID NO: 712), wherein the erythroid cell further presents, e.g. comprises on the cell surface, an exogenous polypeptide comprising a costimulatory polypeptide. In some embodiments, the exogenous antigenic polypeptide is YMLDLQPET (SEQ ID NO: 713). In some embodiments, the costimulatory polypeptide is 4-1BBL.

Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is HPV-E7. Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is YMLDLQPET (SEQ ID NO: 713), YMLDLQPETT (SEQ ID NO: 714), or TIHDIILECV (SEQ ID NO: 712). In some embodiments, the exogenous antigenic polypeptide is YMLDLQPET (SEQ ID NO: 713). In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion.

Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is HPV-E7, wherein the erythroid cell further presents, e.g. comprises on the cell surface, an exogenous polypeptide comprising a costimulatory polypeptide. In some embodiments, the costimulatory polypeptide is 4-1BBL. Also encompassed by the disclosure is an artificial antigen presenting cell (aAPC) comprising an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion, and wherein the exogenous antigenic polypeptide is YMLDLQPET (SEQ ID NO: 713), YMLDLQPETT (SEQ ID NO: 714), or TIHDIILECV (SEQ ID NO: 712), wherein the erythroid cell further presents, e.g. comprises on the cell surface, an exogenous polypeptide comprising a costimulatory polypeptide. In some embodiments, the exogenous antigenic polypeptide is YMLDLQPET (SEQ ID NO: 713). In some embodiments, the costimulatory polypeptide is 4-1BBL. In embodiments, the exogenous antigen-presenting polypeptide is an MHC Class I HLA-A2 polypeptide or single chain fusion. In some embodiments, the exogenous antigen-presenting polypeptide is an MHC Class I HLA-A2 single chain fusion.

In one particular embodiment, an artificial antigen presenting cell comprises an erythroid cell, wherein the erythroid cell presents, e.g. comprises on the cell surface, at least one exogenous antigenic polypeptide, wherein the exogenous antigenic polypeptide is a HPV antigen, fused to an exogenous antigen presenting polypeptide, MHC Class I HLA-A2, fused to the GPA transmembrane domain (GPA). In some embodiments, the HPV antigen is human HPV antigen. In one particular embodiment, an artificial antigen presenting cell comprises an erythroid cell, wherein the erythroid cell presents, e.g. comprises on the cell surface, at least one exogenous antigenic polypeptide, wherein the exogenous antigenic polypeptide is HPV-E7, fused to an exogenous antigen presenting polypeptide, MHC Class I HLA-A2, fused to the GPA transmembrane domain (GPA). In one particular embodiment, an artificial antigen presenting cell comprises an erythroid cell, wherein the erythroid cell presents, e.g. comprises on the cell surface, at least one exogenous antigenic polypeptide, wherein the exogenous antigenic polypeptide is TIHDIILECV (SEQ ID NO: 712), fused to an exogenous antigen presenting polypeptide, MHC Class I HLA-A2, fused to the GPA transmembrane domain (GPA).

An aAPC as described herein, presenting an HPV antigen, can be used in the treatment of a cancer associated with an oncogenic virus (e.g. HPV). An aAPC as described herein, presenting an HPV antigen, and further presenting an exogenous polypeptide comprising a costimulatory polypeptide, can be used in the treatment a cancer associated with an oncogenic virus (e.g. HPV). An aAPC as described herein, presenting an HPV antigen, and further presenting an exogenous polypeptide comprising a costimulatory polypeptide, wherein the costimulatory polypeptide is 4-1BBL, can be used in the treatment of a cancer associated with an oncogenic virus (e.g. HPV), as described in more detail below. In embodiments, the HPV associated cancer is HPV-positive head and neck cancer. In some embodiments, the HPV associated cancer is HPV-positive cervical cancer.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is CD33.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is CD123.

In some embodiments, an artificial antigen presenting cell (aAPC) of the present disclosure comprises an erythroid cell, wherein the erythroid cell presents, e.g. comprises on the cell surface, at least one exogenous antigenic polypeptide, wherein the at least one exogenous antigenic polypeptide is CD38.

In various embodiments of the foregoing aspects, the aAPC presents, e.g. comprises on the cell surface, at least two, at least 3, at least 4, or at least 5 exogenous antigenic polypeptides.

In another aspect, the disclosure features an artificial antigen presenting cell (aAPC) engineered to activate T cells (e.g. cytotoxic CD8+ T cells), wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is from Epstein-Barr Virus (EBV). The aAPC may further comprise an exogenous costimulatory polypeptide as disclosed herein.

In another aspect, the disclosure features an artificial antigen presenting cell (aAPC) engineered to activate T cells (e.g. cytotoxic CD8+ T cells), wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is gp350 or an immunogenic peptide thereof. In a particular embodiment, the immunogenic peptide of gp350 comprises or consists of the HLA A2 peptide (VLQWASLAV (SEQ ID NO: 698)). The aAPC may further comprise an exogenous costimulatory polypeptide as disclosed herein. In a particular embodiment, the exogenous costimulatory polypeptide is 4-1BBL.

In another aspect, the disclosure features an artificial antigen presenting cell (aAPC) engineered to activate T cells (e.g. cytotoxic CD8+ T cells), wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is EBNA1 or an immunogenic peptide thereof. In a particular embodiment, the immunogenic peptide of EBNA1 comprises or consists of a peptide selected from FMVFLQTHI (SEQ ID NO: 699), FLQTHIFAEV (SEQ ID NO: 700), and SIVCYFMVFL (SEQ ID NO: 701). The aAPC may further comprise an exogenous costimulatory polypeptide as disclosed herein. In a particular embodiment, the exogenous costimulatory polypeptide is 4-1BBL.

In another aspect, the disclosure features an artificial antigen presenting cell (aAPC) engineered to expand regulatory T cells (Tregs), wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is from Epstein-Barr Virus (EBV). The aAPC may further comprise an exogenous costimulatory polypeptide as disclosed herein.

In another aspect, the disclosure features an artificial antigen presenting cell (aAPC) engineered to suppress autoreactive T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is from Epstein-Barr Virus (EBV). The aAPC may further comprise an exogenous co-inhibitory polypeptide as disclosed herein.

In another aspect, the disclosure features an aAPC engineered to activate pathogen-specific T cells, comprising an erythroid cell (e.g. an enucleated erythroid cell), wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is an antigenic polypeptide from a pathogen or infectious agent listed in Tables 21-24, or an immunogenic peptide thereof. The aAPC may further comprise an exogenous co-stimulatory polypeptide as disclosed herein.

In another aspect, the disclosure features an aAPC engineered to activate Hepatitis B Virus (HBV)-specific T cells, comprising an erythroid cell (e.g. an enucleated erythroid cell), wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion, and wherein the exogenous antigenic polypeptide is from Hepatitis B Virus (HBV). The aAPC may further comprise, e.g. on the cell surface, an exogenous costimulatory polypeptide as disclosed herein. In embodiments, the at least one costimulatory polypeptide is selected from the group consisting of 4-1BBL, IL-2, IL-12, IL-15, IL-18, IL-21, and any combination thereof, e.g., IL-12 and IL-15, or 4-1BBL and IL-15. In some embodiments, the aAPC further comprises an additional exogenous polypeptide, wherein the additional exogenous polypeptide comprises, e.g. on the cell surface, a checkpoint inhibitor. In some embodiments, the checkpoint inhibitor is an antibody molecule to PD1. In a particular embodiment, the aAPC comprises an erythroid cell, wherein the erythroid cell comprises, e.g. on the cell surface, one or more exogenous polypeptides, wherein the one or more exogenous polypeptides comprise: an exogenous antigenic polypeptide comprising an HBV-specific antigen, or an immunogenic peptide thereof, an exogenous antigen-presenting polypeptide, e.g., MHC class I or MHC class I polypeptide or single chain fusion, an exogenous costimulatory polypeptide, e.g., IL-12 or 4-1BBL, and a checkpoint inhibitor, e.g., antibody to PD1.

### Exogenous Antigen-Presenting Polypeptides

Exogenous antigen-presenting polypeptides of the present disclosure include polypeptides of the MHC gene family, which is divided into three subgroups: class I, class II, and class III.

MHC class I molecules are heterodimers that consist of two polypeptide chains, an α chain and a β2-microglobulin (b2m) chain. The class I α chains consist of a single polypeptide composed of three extracellular domains named α1, α2, and α3, a transmembrane region that anchors it in the plasma membrane, and a short intracytoplasmic tail. The b2m consists of a single molecule noncovalently bound to the α chain. Only the α chain is polymorphic and encoded by a HLA gene, while the b2m subunit is not polymorphic and encoded by the Beta-2 microglobulin gene. Class I MHC molecules have β2 subunits so can only be recognized by CD8 co-receptors.

In some embodiments of the present disclosure, the exogenous antigen-presenting polypeptide comprised in an aAPC is a Class I MHC molecule and includes a signal sequence. In some embodiments, the exogenous antigen-presenting polypeptide is a Class I MHC molecule, and does not include a signal sequence.

MHC class II molecules are also heterodimers that consist of an α and β polypeptide chain. The subdesignation of chains as e.g., α1, α2, and β1 and β2, refers to separate domains (or subunits) within the HLA gene and β gene. CD4 binds to the β2 region. In some embodiments, the exogenous antigen-presenting polypeptide is a Class II MHC molecule and includes a signal sequence. In some embodiments, the exogenous antigen-presenting polypeptide is a Class II MHC molecule, and does not include a signal sequence.

The exogenous antigen-presenting polypeptides of the present disclosure can include subunits of a cell surface complex or cell surface molecule, e.g., MHCI or MHCII, where MHCI or MHCII function to bind an exogenous antigenic polypeptide. In some embodiments, the exogenous antigen-presenting polypeptides are subunits of MHCII, and a function is to bind an exogenous antigenic polypeptide. In some embodiments, the exogenous antigen-presenting polypeptides are subunits of MHCI and a function is to bind an exogenous antigenic polypeptide. In some embodiments, the MHC class I polypeptide or the MHC Class II polypeptide comprises a leader (signal) sequence. In some embodiments, the MHC class I polypeptide or the MHC Class II polypeptide does not comprise a leader (signal) sequence. In some embodiments, a leader sequence is fused to an exogenous antigen presenting polypeptide (e.g., MHC class I or MHC class II polypeptide lacking its leader (signal) sequence). In some embodiments, the MHC Class I polypeptide is a fusion polypeptide comprising a leader sequence. In some embodiments, the MHC Class II polypeptide is a fusion polypeptide comprising a leader sequence. In some embodiments, the leader sequence is selected from the sequences set forth in Table 2.

TABLE 2
Leader Sequences

| SEQ ID | | |
|---|---|---|
| NO. | Sequence Description | Amino Acid Sequence |
| 730 | Beta 2 | MSRSVALAVLALLSLSGLEA |
| | microglobulin (b2m) | |
| | leader sequence | |
| 731 | Glycophorin A | MYGKIIFVLLLSEIVSISA |
| | (GPA) signal peptide | |

In some embodiments, an exogenous antigen-presenting polypeptide is a functional MHC I, and the exogenous antigen-presenting polypeptides are MHC I (alpha chain 1-3) and beta-2 microglobulin, or fragments or variants thereof. In some embodiments an exogenous antigen-presenting polypeptide is a functional MHC II and the exogenous antigen-presenting polypeptides are MHC II alpha chain (alpha chain 1 and 2) and MHC II beta chain (beta chain 1 and 2), or fragments or variants thereof. In some embodiments, the MHC molecule comprises human MHC class I or II, e.g., MHC II alpha subunit and MHC II beta subunit or a fusion molecule comprising both subunits or antigen-presenting fragments thereof. In some embodiments, the HLA α chain is covalently bound (e.g., in a fusion protein with) or non-covalently bound to the β chain.

An aAPC comprising an erythroid cell (e.g., an enucleated erythroid cell) or an enucleated cell, as described herein, with the antigen-presenting polypeptides described herein (e.g. MHC I and MHC II) is used, in some embodiments, for immune induction and/or antigen presentation. In some embodiments, the aAPC comprises a single protein that is a fusion between an MHC molecule and an antigen, e.g., a single-chain peptide-MHC construct comprising an MHC I or MHC II polypeptide and an exogenous antigenic polypeptide. In other embodiments, a non-membrane tethered component of the complex, e.g. the peptide, or the β2 microglobulin, is assembled with another agent within the cell prior to trafficking to the surface, is secreted by the cell and then captured on the surface by the membrane-tethered component of the multimer, or is added in a purified form to an aAPC.

In some embodiments, the antigen-presenting polypeptide comprises both an MHCI α chain and MHC I b2m chain. In some embodiments, the antigen-presenting polypeptide comprises only the MHC I α chain. In some embodiments, the antigen-presenting polypeptide comprises only the MHC I b2m chain. In some embodiments, the antigen-presenting polypeptide comprises both an MHCI α chain and MHC I b2m chain, and the MHC I α chain and MHC I b2m chain are linked non-covalently. In some embodiments, the antigen-presenting polypeptide comprises both an MHCI α chain and MHC I b2m chain, and the MHC I α chain and MHC I b2m chain are linked covalently or fused. In some embodiments, the antigen-presenting polypeptide comprises an MHC I single chain fusion, wherein an exogenous antigenic polypeptide is linked to the MHCI α chain. In some embodiments, the antigen-presenting polypeptide comprises an MHC I single chain fusion, wherein an exogenous antigenic polypeptide is linked to the MHC I b2m chain. In some embodiments, the antigen-presenting polypeptide comprises an MHC I single chain fusion, wherein the exogenous antigenic polypeptide is linked to the MHCI β2m subunit, which is linked to the MHCI α subunit.

In some embodiments, the antigen-presenting polypeptide comprises both the MHC II α chain and MHC II β chain. In some embodiments, the antigen-presenting polypeptide comprises only the MHC II α chain. In some embodiments, the antigen-presenting polypeptide comprises only the MHC II β chain. In some embodiments, the antigen-presenting polypeptide comprises both the MHC II α chain and MHC II β chain, and the MHC II α chain and MHC II β chain are linked non-covalently. In some embodiments, the antigen-presenting polypeptide comprises both the MHC II α chain and MHC II β chain, and the MHC II α chain and MHC II β chain are linked covalently or fused. In some embodiments, the antigen-presenting polypeptide comprises an MHC II single chain fusion, wherein an exogenous antigenic polypeptide is linked to the MHCII α chain. In some embodiments, the antigen-presenting polypeptide comprises an MHC II single chain fusion, wherein an exogenous antigenic polypeptide is linked to the MHC II β chain. In some embodiments, the antigen-presenting polypeptide comprises an MHC II single chain fusion, wherein the exogenous antigenic polypeptide is linked to the MHCII β-chain, which is linked to the MHCII α-chain.

In some embodiments, the MHC I single chain fusion or the MHC II single chain fusion comprises an anchor. In some embodiments, the anchor is a type 1 membrane protein. In some embodiments, the type 1 membrane protein anchor is selected from the group consisting of Glycophorin A (GPA); glycophorin B (GPB); Basigin (also known as CD147); CD44; CD58 (also known as LFA3); Intercellular Adhesion Molecule 4 (ICAM4); Basal Cell Adhesion Molecule (BCAM); CR1; CD99; Erythroblast Membrane Associated Protein (ERMAP); junctional adhesion molecule A (JAM-A); neuroplastin (NPTN); AMIGO2; and DS Cell Adhesion Molecule Like 1 (DSCAML1). In some embodiments, the anchor is a type 2 membrane protein. In some embodiments, the type 2 membrane protein anchor is selected from the group consisting of small integral membrane protein 1 (SMIM1), transferrin receptor (CD71); FasL transmembrane; and Kell. In some embodiments, the anchor is a GPI-linked membrane protein. In some embodiments, the GPI-linked membrane protein anchor is selected from the group consisting of CD59; CD55; and Semaphorin 7A (SEMA7A). In some embodiments, the anchor is small integral membrane protein 1 (SMIM1). In some embodiments, the anchor is glycophorin anchor, and in particular glycophorin A (GPA), or a fragment thereof. In some embodiments, the anchor is selected from an amino acid sequence listed in Table 3.

TABLE 3
Anchor Sequences

| SEQ | | | |
|---|---|---|---|
| ID | Sequence | Sequence | |
| NO: | name | description | Amino acid sequence |
| 727 | GPA | Full length GPA | MYGKIIFVLLLSAIVSISALSTTEVAMHTSTSS |
| | | | SVTKSYISSQTNDTHKRDTYAATPRAHEVSEI |
| | | | SVRTVYPPEEETGERVQLAHHFSEPEITLIIFG |
| | | | VMAGVIGTILLISYGIRRLIKKSPSDVKPLPSP |
| | | | DTDVPLSSVEIENPETSDQ |
| 728 | GPA | Fragment of GPA | LSTTEVAMHTSTSSSVTKSYISSQTNDTHKR |
| | | comprising a | DTYAATPRAHEVSEISVRTVYPPEEETGERV |
| | | transmembrane | QLAHHFSEPEITLIIFGVMAGVIGTILLISYGIR |
| | | domain | RLIKKSPSDVKPLPSPDTDVPLSSVEIENPETS |
| | | | DQ |
| 729 | SMIM1 | SMIM1 | MQPQESHVHYSRWEDGSRDGVSLGAVSSTE |
| | | | EASRCRRISQRLCTGKLGIAMKVLGGVALF |
| | | | WIIFILGYLTGYYVHKCK |

In some embodiments, the exogenous antigenic polypeptide is connected to the MHC class I or MHC class II single chain fusion via a linker. In some embodiments, the MHC class I or MHC class II single chain fusion is connected to an anchor sequence via a linker. In one embodiment, the linker is a cleavable linker. In some embodiments, the linker is selected from an amino acid sequence listed in Table 4.

TABLE 4
Linker Sequences

| SEQ ID | Sequence | |
|---|---|---|
| NO. | Description | Amino Acid Sequence |
| 732 | Linker | GGGGSGGGGSGGGGS |
| 733 | Linker | GGGGSGGGGSGGGGSGGGGS |
| 734 | Linker | GSGSGSGSEDGSGSGSGS |
| 735 | Linkr | GSGSGSGSGSGSGSGSGS |
| 736 | Linker | GCGGSGGGGSGGGGS |
| 737 | Linker | GGSGGSGGGGGSGGGSGGGSGGGS |
| 738 | Linker | SGRGGGGSGGGGSGGGGSGGGGSSPA |
| 739 | Linker | GGGGSGGGGSGGGGSGGGGSGGGG |
| 740 | Snorkel linker | SGRGASSGSSGSGSQKKPRYEIRWKVVVI |
| | | SAILALVVLTVISLIILIMLWGSGMQSPA |
| | | (“snorkel linker”) |

In some embodiments, the exogenous antigenic polypeptide is loaded on the MHCI molecule, and a function is to present the exogenous antigenic polypeptide. In some embodiments, the exogenous antigenic polypeptide is loaded on the MHCII molecule, and a function is to present the exogenous antigenic polypeptide. In some embodiments, the exogenous antigenic polypeptides are presented on antigen-presenting polypeptides, e.g., the exogenous antigenic polypeptide is specifically bound to the exogenous antigen-presenting polypeptides, e.g. MHC class I and/or class II molecules. The exogenous antigenic polypeptide may be bound either covalently or non-covalently to the antigen-presenting polypeptide. In some embodiments, the exogenous antigenic peptide is free, and can be specifically bound to the antigen-presenting polypeptide present on cell surface of the artificial antigen presenting cell. In some embodiments, coupling reagents can be used to link an exogenous polypeptide to an antigen-presenting polypeptide present on the cell surface. In some embodiments, click chemistry, as described in detail herein, can be used to link an exogenous polypeptide to an antigen-presenting polypeptide present on the cell surface.

Multiple assays for assessing binding affinity and/or determining whether an antigenic polypeptide specifically binds to a particular ligand (e.g., an MHC molecule) are known in the art. For example, in some embodiments, surface plasmon resonance (Biacore®) can be used to determine the binding constant of a complex between two polypeptides. In this assay, the dissociation constant for the complex can be determined by monitoring changes in the refractive index with respect to time as buffer is passed over the chip. Other suitable assays for measuring the binding of one polypeptide to another include, for example, immunoassays such as enzyme linked immunosorbent assays (ELISA) and radioimmunoassays (RIA), or determination of binding by monitoring the change in the spectroscopic or optical properties of the proteins using fluorescence, UV absorption, circular dichroism, or nuclear magnetic resonance (NMR). Other exemplary assays include, but are not limited to, Western blot, analytical ultracentrifugation, and spectroscopy (see, e.g., Scatchard et al (1949) Ann. N.Y. Acad. Sci. 51:660; Wilson (2002) Science 295: 2103; Woffi et al. (1993) Cancer Res. 53:2560; U.S. Pat. Nos. 5,283,173, 5,468,614; and International Patent Publication No. WO 2018/005559. Alternatively, binding of an antigenic polypeptide to a particular ligand (e.g., an MHC molecule) may be determined using a predictive algorithm. For example, methods for predicting MHC class II and class II epitopes are well known in the art, and include TEPITOPE (see, e.g., Meister et al. (1995) Vaccine 13: 581-91), EpiMatrix (De Groot et al. (1997) AIDS Res Hum Retroviruses 13: 529-31), the Predict Method (Yu et al. (2002) Mol. Med. 8: 137-48), the SYFPEITHI epitope prediction algorithm (Schuler et al. (2007) Methods Mol Biol. 409: 75-93, and Rankpep (Reche et al. (2002) Hum Immunol. 63(9): 701-9). Additional algorithms for predicting MHC class I and class II epitopes are described, for example, in Kessler and Melief (2007) Leukemia 21(9): 1859-74.

In some embodiments, an exogenous antigen-presenting polypeptide is selected from the group consisting of HLA-A, HLA-B, HLA-C, HLA-DRB1, HLA-DQA1, HLA-DQB1, HLA-DPA1, HLA-DPB1, that are capable of binding antigens and displaying them on the cell surface.

In some embodiments, the exogenous antigen-presenting polypeptide is an MHC class I polypeptide or an MHC class I single chain fusion. In a further embodiment, the MHC class I polypeptide is HLA-A. In some embodiments, an HLA-A polypeptide comprises an HLA-A single chain fusion polypeptide, wherein the HLA-A polypeptide is linked to an exogenous antigenic polypeptide. In some embodiments, the HLA-A single chain fusion polypeptide includes a membrane anchor. In some embodiments, the membrane anchor is selected from a membrane anchor set forth in Table 3. In some embodiments, the HLA-A single chain fusion polypeptide includes a linker (e.g., between the antigenic peptide and beta chain, between the beta chain and alpha chain, or between the alpha chain and anchor). In some embodiments, the linker is selected from a sequence set forth in Table 4. In some embodiments, the HLA-A single chain fusion polypeptide includes a leader sequence. In some embodiments, the leader sequence is selected from a sequence set forth in Table 2 In some embodiments, an HLA-A leader sequence is fused to an exogenous antigen-presenting polypeptide, which is linked to a membrane anchor.

In some embodiments, the exogenous antigen-presenting polypeptide is an MHC class I polypeptide or an MHC class I single chain fusion. In a further embodiment, the MHC class I polypeptide is HLA-B. In some embodiments, an HLA-B polypeptide comprises an HLA-B single chain fusion polypeptide, wherein the HLA-B polypeptide is linked to an exogenous antigenic polypeptide. In some embodiments, the HLA-B single chain fusion polypeptide includes a membrane anchor. In some embodiments, the membrane anchor is selected from a membrane anchor set forth in Table 3. In some embodiments, the HLA-B single chain fusion polypeptide includes a linker (e.g., between the antigenic peptide and beta chain, between the beta chain and alpha chain, or between the alpha chain and anchor). In some embodiments, the linker is selected from a sequence set forth in Table 4. In some embodiments, the HLA-B single chain fusion polypeptide includes a leader sequence. In some embodiments, the leader sequence is selected from a sequence set forth in Table 2. In some embodiments, an HLA-B leader sequence is fused to an exogenous antigen-presenting polypeptide, which is linked to a membrane anchor.

In some embodiments, the exogenous antigen-presenting polypeptide is an MHC class I polypeptide or an MHC class I single chain fusion. In a further embodiment, the MHC class I polypeptide is HLA-C. In some embodiments, an HLA-C polypeptide comprises an HLA-C single chain fusion polypeptide, wherein the HLA-C polypeptide is linked to an exogenous antigenic polypeptide. In some embodiments, the HLA-C single chain fusion polypeptide includes a membrane anchor. In some embodiments, the membrane anchor is selected from a sequence set forth in Table 3. In some embodiments, the HLA-C single chain fusion polypeptide includes a linker (e.g., between the antigenic peptide and beta chain, between the beta chain and alpha chain, or between the alpha chain and anchor). In some embodiments, the linker is selected from a sequence set forth in Table 4. In some embodiments, the HLA-C single chain fusion polypeptide includes a leader sequence. In some embodiments, the leader sequence is selected from a sequence set forth in Table 2. In some embodiments, an HLA-C leader sequence is fused to an exogenous antigen-presenting polypeptide, which is linked to a membrane anchor.

In some embodiments, the exogenous antigen-presenting polypeptide is an MHC class II polypeptide or an MHC class II single chain fusion. In a further embodiment, the MHC class II polypeptide is selected from the group consisting of HLA-DPα, HLA-DPβ, HLA-DM, HLA DOA, HLA-DOB, HLA-DQα, HLA-DQβ, HLA-DRα, and HLA-DRβ. In some embodiments, an HLA-DPA polypeptide comprises an HLA-DPA single chain fusion polypeptide, wherein the HLA-DPA polypeptide is linked to an exogenous antigenic polypeptide. In some embodiments, an HLA-DPB polypeptide comprises an HLA-DPB single chain fusion polypeptide, wherein the HLA-DPB polypeptide is linked to an exogenous antigenic polypeptide. In some embodiments, an HLA-DM polypeptide comprises an HLA-DM single chain fusion polypeptide, wherein the HLA-DM polypeptide is linked to an exogenous antigenic polypeptide. In some embodiments, an HLA-DOA polypeptide comprises an HLA-DOA single chain fusion polypeptide, wherein the HLA-DOA polypeptide is linked to an exogenous antigenic polypeptide. In some embodiments, an HLA-DOB polypeptide comprises an HLA-DOB single chain fusion polypeptide, wherein the HLA-DOB polypeptide is linked to an exogenous antigenic polypeptide. In some embodiments, an HLA-DQA polypeptide comprises an HLA-DQA single chain fusion polypeptide, wherein the HLA-DQA polypeptide is linked to an exogenous antigenic polypeptide. In some embodiments, an HLA-DQB polypeptide comprises an HLA-DQB single chain fusion polypeptide, wherein the HLA-DQB polypeptide is linked to an exogenous antigenic polypeptide. In some embodiments, an HLA-DRA polypeptide comprises an HLA-DRA single chain fusion polypeptide, wherein the HLA-DRA polypeptide is linked to an exogenous antigenic polypeptide. In some embodiments, an HLA-DRB polypeptide comprises an HLA-DRB single chain fusion polypeptide, wherein the HLA-DRB polypeptide is linked to an exogenous antigenic polypeptide. In some embodiments, the single chain fusion polypeptides include a membrane anchor. In some embodiments, the membrane anchor is selected from a sequence set forth in Table 3. In some embodiments, the single chain fusion polypeptides include a linker (e.g., between the antigenic peptide and beta chain, between the beta chain and alpha chain, or between the alpha chain and anchor). In some embodiments, the linker is selected from a sequence set forth in Table 4. In some embodiments, the single chain fusion polypeptides include a leader sequence. In some embodiments, the leader sequence is selected from a sequence set forth in Table 2.

The protein products of MHC class I and class II genes are known to be highly polymorphic, thus the present disclosure also encompasses MHC polymorphs. There are more than 200 alleles of some human MHC class I and class II genes. With the exception of the DRα locus, which is functionally monomorphic, each locus has many alleles (Janeway C A Jr, Travers P, Walport M, et al. Immunobiology: The Immune System in Health and Disease. 5th edition. New York: Garland Science; 2001, incorporated by reference in its entirety herein), each allele being present at a relatively high frequency in the population.

In some embodiments, the MHC class I polypeptide is HLA-A. In some embodiments, the HLA-A polypeptide comprises an HLA-A allele selected from the group consisting of A*01:01, A*02:01, A *03:01, A*24:02, A*11:01, A*29:02, A*32:01, A*68:01, A*31:01, A*25:01, A*26:01, A*23:01, A*30:01.

In some embodiments, the MHC class I polypeptide is HLA-B. In some embodiments, the HLA-B polypeptide comprises an HLA-B allele selected from the group consisting of B*08:01, B*07:02, B*44:02, B*15:01, B*40:01, B*44:03, B*35:01, B*51:01, B*27:05, B*57:01, B*18:01, B*14:02, B*13:02, B*55:01, B*14:01, B*49:01, B*37:01, B*38:01, B*39:01, B*35:03, B*40:02.

In some embodiments, the MHC class I polypeptide is HLA-C. In some embodiments, the HLA-C polypeptide comprises an HLA-C allele selected from the group consisting of C*07:01, C*07:02, C*05:01, C*06:02, C*04:01, C*03:04, C*03:03, C*02:02, C*16:01, C*08:02, C*12:03, C*01:02, C*15:02, C*07:04, C*14:02.

In some embodiments, the MHC class II polypeptide is selected from the group consisting of HLA-DPα, HLA-DPβ, HLA-DM, HLA DOA, HLA-DOB, HLA-DQα, HLA-DQβ, HLA-DRα, and HLA-DRβ. In some embodiments, the HLA-DPα polypeptide comprises an HLA-DPα allele selected from the group consisting of DPA1*01:03, DPA1*02:01, DPA1*02:07. In some embodiments, the HLA-DPβ polypeptide comprises an HLA-DPβ allele selected from the group consisting of DPB1*04:01, DPB1*02:01, DPB1*04:02, DPB1*03:01, DPB1*01:01, DPB1*11:01, DPB1*05:01, DPB1*10:01, DPB1*06:01, DPB1*13:01, DPB1*14:01, and DPB1*17:01. In some embodiments, the HLA-DQα polypeptide comprises an HLA-DQα allele selected from the group consisting of DQA1*05:01, DQA1*03:01, DQA1*01:02, DQA1*02:01, DQA1*01:01, DQA1*01:03, and DQA1*04:01. In some embodiments, the HLA-DQβ polypeptide comprises an HLA-DQβ allele selected from the group consisting of DQB1*03:01, DQB1*02:01, DQB1*06:02, DQB1*05:01, DQB1*02:02, DQB1*03:02, DQB1*06:03, DQB1*03:03, DQB1*06:04, DQB1*05:03, and DQB1*04:02. In some embodiments, the HLA-DRβ polypeptide comprises an HLA-DRβ allele selected from the group consisting of DRB1*07:01, DRB1*03:01, DRB1*15:01, DRB1*04:01, DRB1*01:01, DRB1*13:01, DRB1*11:01, DRB1*04:04, DRB1*13:02, DRB1*08:01, DRB1*12:01, DRB1*11:04, DRB1*09:01, DRB1*14:01, DRB1*04:07, and DRB1*14:04.

In some embodiments, an antigen-presenting polypeptide comprises an HLA allele polypeptide comprising or consisting of an amino acid sequence set forth in Table 5. In some embodiments, the MHC allele polypeptide comprises a signal peptide. In other embodiments, the MHC allele polypeptide does not include a signal peptide. Accordingly, in some embodiments, the antigen-presenting polypeptide comprises the amino acid sequence of any one of SEQ ID NOs 741-838, shown in Table 5, excluding the signal peptide amino acid sequence (shown underlined in the sequences in Table 5). In other embodiments, the antigen-presenting polypeptide comprises the amino acid sequence of any one of SEQ ID NOs 741-838 shown in Table 5 including the signal peptide amino acid sequence (shown underlined in Table 5).

TABLE 5
HLA Alleles

| | | |
|---|---|---|
| A*01:01 | MAVMAPRTLLLLLSGALALTQTWAGSHSMRYFF | (SEQ ID NO: |
| (IMGT/HLA | TSVSRPGRGEPRFIAVGYVDDTQFVRFDSDAASQ | 741) |
| Accession No. | KMEPRAPWIEQEGPEYWDQETRNMKAHSQTDR | |
| HLA00001) | ANLGTLRGYYNQSEDGSHTIQIMYGCDVGPDGR | |
| * Predicted signal | FLRGYRQDAYDGKDYIALNEDLRSWTAADMAA | |
| peptide underlined | QITKRKWEAVHAAEQRRVYLEGRCVDGLRRYL | |
| | ENGKETLQRTDPPKTHMTHHPISDHEATLRCWA | |
| | LGFYPAEITLTWQRDGEDQTQDTELVETRPAGD | |
| | GTFQKWAAVVVPSGEEQRYTCHVQHEGLPKPLT | |
| | LRWELSSQPTIPIVGIIAGLVLLGAVITGAVVAAV | |
| | MWRRKSSDRKGGSYTQAASSDSAQGSDVSLTAC | |
| | KV | |
| A*02:01 | MAVMAPRTLVLLLSGALALTQTWAGSHSMRYF | (SEQ ID NO: |
| (IMGT/HLA | FTSVSRPGRGEPRFIAVGYVDDTQFVRFDSDAAS | 742) |
| Accession No. | QRMEPRAPWIEQEGPEYWDGETRKVKAHSQTH | |
| HLA00005) | RVDLGTLRGYYNQSEAGSHTVQRMYGCDVGSD | |
| * Predicted signal | WRFLRGYHQYAYDGKDYIALKEDLRSWTAADM | |
| peptide underlined | AAQTTKHKWEAAHVAEQLRAYLEGTCVEWLRR | |
| | YLENGKETLQRTDAPKTHMTHHAVSDHEATLRC | |
| | WALSFYPAEITLTWQRDGEDQTQDTELVETRPA | |
| | GDGTFQKWAAVVVPSGQEQRYTCHVQHEGLPK | |
| | PLTLRWEPSSQPTIPIVGIIAGLVLFGAVITGAVVA | |
| | AVMWRRKSSDRKGGSYSQAASSDSAQGSDVSLT | |
| | ACKV | |
| A*03:01 | MAVMAPRTLLLLLSGALALTQTWAGSHSMRYFF | (SEQ ID NO: |
| (IMGT/HLA | TSVSRPGRGEPRFIAVGYVDDTQFVRFDSDAASQ | 743) |
| Accession No. | RMEPRAPWIEQEGPEYWDQETRNVKAQSQTDR | |
| HLA00037) | VDLGTLRGYYNQSEAGSHTIQIMYGCDVGSDGR | |
| * Predicted signal | FLRGYRQDAYDGKDYIALNEDLRSWTAADMAA | |
| peptide underlined | QITKRKWEAAHEAEQLRAYLDGTCVEWLRRYL | |
| | ENGKETLQRTDPPKTHMTHHPISDHEATLRCWA | |
| | LGFYPAEITLTWQRDGEDQTQDTELVETRPAGD | |
| | GTFQKWAAVVVPSGEEQRYTCHVQHEGLPKPLT | |
| | LRWELSSQPTIPIVGIIAGLVLLGAVITGAVVAAV | |
| | MWRRKSSDRKGGSYTQAASSDSAQGSDVSLTAC | |
| | KV | |
| A*24:02 | MAVMAPRTLVLLLSGALALTQTWAGSHSMRYF | (SEQ ID NO: |
| (IMGT/HLA | STSVSRPGRGEPRFIAVGYVDDTQFVRFDSDAAS | 744) |
| Accession No. | QRMEPRAPWIEQEGPEYWDEETGKVKAHSQTDR | |
| HLA00050) | ENLRIALRYYNQSEAGSHTLQMMFGCDVGSDGR | |
| * Predicted signal | FLRGYHQYAYDGKDYIALKEDLRSWTAADMAA | |
| peptide underlined | QITKRKWEAAHVAEQQRAYLEGTCVDGLRRYL | |
| | ENGKETLQRTDPPKTHMTHHPISDHEATLRCWA | |
| | LGFYPAEITLTWQRDGEDQTQDTELVETRPAGD | |
| | GTFQKWAAVVVPSGEEQRYTCHVQHEGLPKPLT | |
| | LRWEPSSQPTVPIVGIIAGLVLLGAVITGAVVAAV | |
| | MWRRNSSDRKGGSYSQAASSDSAQGSDVSLTAC | |
| | KV | |
| A*11:01 | MAVMAPRTLLLLLSGALALTQTWAGSHSMRYF | (SEQ ID NO: |
| (IMGT/HLA | YTSVSRPGRGEPRFIAVGYVDDTQFVRFDSDAAS | 745) |
| Accession No. | QRMEPRAPWIEQEGPEYWDQETRNVKAQSQTD | |
| HLA00043) | RVDLGTLRGYYNQSEDGSHTIQIMYGCDVGPDG | |
| * Predicted signal | RFLRGYRQDAYDGKDYIALNEDLRSWTAADMA | |
| peptide underlined | AQITKRKWEAAHAAEQQRAYLEGRCVEWLRRY | |
| | LENGKETLQRTDPPKTHMTHHPISDHEATLRCW | |
| | ALGFYPAEITLTWQRDGEDQTQDTELVETRPAG | |
| | DGTFQKWAAVVVPSGEEQRYTCHVQHEGLPKPL | |
| | TLRWELSSQPTIPIVGIIAGLVLLGAVITGAVVAA | |
| | VMWRRKSSDRKGGSYTQAASSDSAQGSDVSLT | |
| | ACKV | |
| A*29:02 | MAVMAPRTLLLLLLGALALTQTWAGSHSMRYF | (SEQ ID NO: |
| (IMGT/HLA | TTSVSRPGRGEPRFIAVGYVDDTQFVRFDSDAAS | 746) |
| Accession No. | QRMEPRAPWIEQEGPEYWDLQTRNVKAQSQTD | |
| HLA00086) | RANLGTLRGYYNQSEAGSHTIQMMYGCDVGSD | |
| * Predicted signal | GRFLRGYRQDAYDGKDYIALNEDLRSWTAADM | |
| peptide underlined | AAQITQRKWEAARVAEQLRAYLEGTCVEWLRR | |
| | YLENGKETLQRTDAPKTHMTHHAVSDHEATLRC | |
| | WALSFYPAEITLTWQRDGEDQTQDTELVETRPA | |
| | GDGTFQKWASVVVPSGQEQRYTCHVQHEGLPK | |
| | PLTLRWEPSSQPTIPIVGIIAGLVLFGAVFAGAVV | |
| | AAVRWRRKSSDRKGGSYSQAASSDSAQGSDMS | |
| | LTACKV | |
| A*32:01 | MAVMAPRTLLLLLLGALALTQTWAGSHSMRYF | (SEQ ID NO: |
| (IMGT/HLA | FTSVSRPGRGEPRFIAVGYVDDTQFVRFDSDAAS | 747) |
| Accession No. | QRMEPRAPWIEQEGPEYWDQETRNVKAHSQTD | |
| HLA00101) | RESLRIALRYYNQSEAGSHTIQMMYGCDVGPDG | |
| * Predicted signal | RLLRGYQQDAYDGKDYIALNEDLRSWTAADMA | |
| peptide underlined | AQITQRKWEAARVAEQLRAYLEGTCVEWLRRY | |
| | LENGKETLQRTDAPKTHMTHHAVSDHEATLRC | |
| | WALSFYPAEITLTWQRDGEDQTQDTELVETRPA | |
| | GDGTFQKWASVVVPSGQEQRYTCHVQHEGLPK | |
| | PLTLRWEPSSQPTIPIVGIIAGLVLFGAMFAGAVV | |
| | AAVRWRRKSSDRKGGSYSQAASSDSAQGSDMS | |
| | LTACKV | |
| A*68:01 | MAVMAPRTLVLLLSGALALTQTWAGSHSMRYF | (SEQ ID NO: |
| (IMGT/HLA | YTSVSRPGRGEPRFIAVGYVDDTQFVRFDSDAAS | 748) |
| Accession No. | QRMEPRAPWIEQEGPEYWDRNTRNVKAQSQTD | |
| HLA00115) | RVDLGTLRGYYNQSEAGSHTIQMMYGCDVGSD | |
| * Predicted signal | GRFLRGYRQDAYDGKDYIALKEDLRSWTAADM | |
| peptide underlined | AAQTTKHKWEAAHVAEQWRAYLEGTCVEWLR | |
| | RYLENGKETLQRTDAPKTHMTHHAVSDHEATLR | |
| | CWALSFYPAEITLTWQRDGEDQTQDTELVETRP | |
| | AGDGTFQKWVAVVVPSGQEQRYTCHVQHEGLP | |
| | KPLTLRWEPSSQPTIPIVGIIAGLVLFGAVITGAVV | |
| | AAVMWRRKSSDRKGGSYSQAASSDSAQGSDVS | |
| | LTACKV | |
| A*31:01 | MAVMAPRTLLLLLLGALALTQTWAGSHSMRYF | (SEQ ID NO: |
| (IMGT/HLA | TTSVSRPGRGEPRFIAVGYVDDTQFVRFDSDAAS | 749) |
| Accession No. | QRMEPRAPWIEQERPEYWDQETRNVKAHSQIDR | |
| HLA00092) | VDLGTLRGYYNQSEAGSHTIQMMYGCDVGSDG | |
| * Predicted signal | RFLRGYQQDAYDGKDYIALNEDLRSWTAADMA | |
| peptide underlined | AQITQRKWEAARVAEQLRAYLEGTCVEWLRRY | |
| | LENGKETLQRTDPPKTHMTHHAVSDHEATLRCW | |
| | ALSFYPAEITLTWQRDGEDQTQDTELVETRPAGD | |
| | GTFQKWASVVVPSGQEQRYTCHVQHEGLPKPLT | |
| | LRWEPSSQPTIPIVGIIAGLVLFGAVFAGAVVAAV | |
| | RWRRKSSDRKGGSYSQAASSDSAQGSDMSLTAC | |
| | KV | |
| A*25:01 | MAVMAPRTLVLLLSGALALTQTWAGSHSMRYF | (SEQ ID NO: |
| (IMGT/HLA | YTSVSRPGRGEPRFIAVGYVDDTQFVRFDSDAAS | 750) |
| Accession No. | QRMEPRAPWIEQEGPEYWDRNTRNVKAHSQTD | |
| HLA00071) | RESLRIALRYYNQSEDGSHTIQRMYGCDVGPDG | |
| * Predicted signal | RFLRGYQQDAYDGKDYIALNEDLRSWTAADMA | |
| peptide underlined | AQITQRKWETAHEAEQWRAYLEGRCVEWLRRY | |
| | LENGKETLQRTDAPKTHMTHHAVSDHEATLRC | |
| | WALSFYPAEITLTWQRDGEDQTQDTELVETRPA | |
| | GDGTFQKWASVVVPSGQEQRYTCHVQHEGLPK | |
| | PLTLRWEPSSQPTIPIVGIIAGLVLFGAVIAGAVVA | |
| | AVMWRRKSSDRKGGSYSQAASSDSAQGSDMSL | |
| | TACKV | |
| A*26:01 | MAVMAPRTLVLLLSGALALTQTWAGSHSMRYF | (SEQ ID NO: |
| (IMGT/HLA | YTSVSRPGRGEPRFIAVGYVDDTQFVRFDSDAAS | 751) |
| Accession No. | QRMEPRAPWIEQEGPEYWDRNTRNVKAHSQTD | |
| HLA00073) | RANLGTLRGYYNQSEDGSHTIQRMYGCDVGPDG | |
| * Predicted signal | RFLRGYQQDAYDGKDYIALNEDLRSWTAADMA | |
| peptide underlined | AQITQRKWETAHEAEQWRAYLEGRCVEWLRRY | |
| | LENGKETLQRTDAPKTHMTHHAVSDHEATLRC | |
| | WALSFYPAEITLTWQRDGEDQTQDTELVETRPA | |
| | GDGTFQKWASVVVPSGQEQRYTCHVQHEGLPK | |
| | PLTLRWEPSSQPTIPIVGIIAGLVLFGAVIAGAVVA | |
| | AVMWRRKSSDRKGGSYSQAASSDSAQGSDMSL | |
| | TACKV | |
| A*23:01 | MAVMAPRTLVLLLSGALALTQTWAGSHSMRYF | (SEQ ID NO: |
| (IMGT/HLA | STSVSRPGRGEPRFIAVGYVDDTQFVRFDSDAAS | 752) |
| Accession No. | QRMEPRAPWIEQEGPEYWDEETGKVKAHSQTDR | |
| HLA00048) | ENLRIALRYYNQSEAGSHTLQMMFGCDVGSDGR | |
| * Predicted signal | FLRGYHQYAYDGKDYIALKEDLRSWTAADMAA | |
| peptide underlined | QITQRKWEAARVAEQLRAYLEGTCVDGLRRYLE | |
| | NGKETLQRTDPPKTHMTHHPISDHEATLRCWAL | |
| | GFYPAEITLTWQRDGEDQTQDTELVETRPAGDG | |
| | TFQKWAAVVVPSGEEQRYTCHVQHEGLPKPLTL | |
| | RWEPSSQPTVHIVGIIAGLVLLGAVITGAVVAAV | |
| | MWRRNSSDRKGGSYSQAASSDSAQGSDVSLTAC | |
| | KV | |
| A*30:01 | MAVMAPRTLLLLLSGALALTQTWAGSHSMRYFS | (SEQ ID NO: |
| (IMGT/HLA | TSVSRPGSGEPRFIAVGYVDDTQFVRFDSDAASQ | 753) |
| Accession No. | RMEPRAPWIEQERPEYWDQETRNVKAQSQTDRV | |
| HLA00089) | DLGTLRGYYNQSEAGSHTIQIMYGCDVGSDGRF | |
| * Predicted signal | LRGYEQHAYDGKDYIALNEDLRSWTAADMAAQ | |
| peptide underlined | ITQRKWEAARWAEQLRAYLEGTCVEWLRRYLE | |
| | NGKETLQRTDPPKTHMTHHPISDHEATLRCWAL | |
| | GFYPAEITLTWQRDGEDQTQDTELVETRPAGDG | |
| | TFQKWAAVVVPSGEEQRYTCHVQHEGLPKPLTL | |
| | RWELSSQPTIPIVGIIAGLVLLGAVITGAVVAAVM | |
| | WRRKSSDRKGGSYTQAASSDSAQGSDVSLTACKV | |
| C*07:01 | MRVMAPRALLLLLSGGLALTETWACSHSMRYF | (SEQ ID NO: |
| (IMGT/HLA | DTAVSRPGRGEPRFISVGYVDDTQFVRFDSDAAS | 754) |
| Accession No. | PRGEPRAPWVEQEGPEYWDRETQNYKRQAQAD | |
| HLA00433) | RVSLRNLRGYYNQSEDGSHTLQRMYGCDLGPD | |
| * Predicted signal | GRLLRGYDQSAYDGKDYIALNEDLRSWTAADT | |
| peptide underlined | AAQITQRKLEAARAAEQLRAYLEGTCVEWLRRY | |
| | LENGKETLQRAEPPKTHVTHHPLSDHEATLRCW | |
| | ALGFYPAEITLTWQRDGEDQTQDTELVETRPAG | |
| | DGTFQKWAAVVVPSGQEQRYTCHMQHEGLQEP | |
| | LTLSWEPSSQPTIPIMGIVAGLAVLVVLAVLGAV | |
| | VTAMMCRRKSSGGKGGSCSQAACSNSAQGSDES | |
| | LITCKA | |
| C*07:02 | MRVMAPRALLLLLSGGLALTETWACSHSMRYF | (SEQ ID NO: |
| (IMGT/HLA | DTAVSRPGRGEPRFISVGYVDDTQFVRFDSDAAS | 755) |
| Accession No. | PRGEPRAPWVEQEGPEYWDRETQKYKRQAQAD | |
| HLA00434) | RVSLRNLRGYYNQSEDGSHTLQRMSGCDLGPDG | |
| * Predicted signal | RLLRGYDQSAYDGKDYIALNEDLRSWTAADTA | |
| peptide underlined | AQITQRKLEAARAAEQLRAYLEGTCVEWLRRYL | |
| | ENGKETLQRAEPPKTHVTHHPLSDHEATLRCWA | |
| | LGFYPAEITLTWQRDGEDQTQDTELVETRPAGD | |
| | GTFQKWAAVVVPSGQEQRYTCHMQHEGLQEPL | |
| | TLSWEPSSQPTIPIMGIVAGLAVLVVLAVLGAVV | |
| | TAMMCRRKSSGGKGGSCSQAACSNSAQGSDESL | |
| | ITCKA | |
| C*05:01 | MRVMAPRTLILLLSGALALTETWACSHSMRYFY | (SEQ ID NO: |
| (IMGT/HLA | TAVSRPGRGEPRFIAVGYVDDTQFVQFDSDAASP | 756) |
| Accession No. | RGEPRAPWVEQEGPEYWDRETQKYKRQAQTDR | |
| HLA00427) | VNLRKLRGYYNQSEAGSHTLQRMYGCDLGPDG | |
| * Predicted signal | RLLRGYNQFAYDGKDYIALNEDLRSWTAADKA | |
| peptide underlined | AQITQRKWEAAREAEQRRAYLEGTCVEWLRRY | |
| | LENGKKTLQRAEHPKTHVTHHPVSDHEATLRCW | |
| | ALGFYPAEITLTWQRDGEDQTQDTELVETRPAG | |
| | DGTFQKWAAVVVPSGEEQRYTCHVQHEGLPEPL | |
| | TLRWGPSSQPTIPIVGIVAGLAVLAVLAVLGAVM | |
| | AVVMCRRKSSGGKGGSCSQAASSNSAQGSDESL | |
| | IACKA | |
| C*06:02 | MRVMAPRTLILLLSGALALTETWACSHSMRYFD | (SEQ ID NO: |
| (IMGT/HLA | TAVSRPGRGEPRFISVGYVDDTQFVRFDSDAASP | 757) |
| Accession No. | RGEPRAPWVEQEGPEYWDRETQKYKRQAQADR | |
| HLA00430) | VNLRKLRGYYNQSEDGSHTLQWMYGCDLGPDG | |
| * Predicted signal | RLLRGYDQSAYDGKDYIALNEDLRSWTAADTA | |
| peptide underlined | AQITQRKWEAAREAEQWRAYLEGTCVEWLRRY | |
| | LENGKETLQRAEHPKTHVTHHPVSDHEATLRCW | |
| | ALGFYPAEITLTWQRDGEDQTQDTELVETRPAG | |
| | DGTFQKWAAVVVPSGEEQRYTCHVQHEGLPEPL | |
| | TLRWEPSSQPTIPIVGIVAGLAVLAVLAVLGAVM | |
| | AVVMCRRKSSGGKGGSCSQAASSNSAQGSDESL | |
| | IACKA | |
| C*04:01 | MRVMAPRTLILLLSGALALTETWAGSHSMRYFS | (SEQ ID NO: |
| (IMGT/HLA | TSVSWPGRGEPRFIAVGYVDDTQFVRFDSDAASP | 758) |
| Accession No. | RGEPREPWVEQEGPEYWDRETQKYKRQAQADR | |
| HLA00420) | VNLRKLRGYYNQSEDGSHTLQRMFGCDLGPDG | |
| * Predicted signal | RLLRGYNQFAYDGKDYIALNEDLRSWTAADTA | |
| peptide underlined | AQITQRKWEAAREAEQRRAYLEGTCVEWLRRY | |
| | LENGKETLQRAEHPKTHVTHHPVSDHEATLRCW | |
| | ALGFYPAEITLTWQWDGEDQTQDTELVETRPAG | |
| | DGTFQKWAAVVVPSGEEQRYTCHVQHEGLPEPL | |
| | TLRWKPSSQPTIPIVGIVAGLAVLAVLAVLGAMV | |
| | AVVMCRRKSSGGKGGSCSQAASSNSAQGSDESL | |
| | IACKA | |
| C*03:04 | MRVMAPRTLILLLSGALALTETWAGSHSMRYFY | (SEQ ID NO: |
| (IMGT/HLA | TAVSRPGRGEPHFIAVGYVDDTQFVRFDSDAASP | 759) |
| Accession No. | RGEPRAPWVEQEGPEYWDRETQKYKRQAQTDR | |
| HLA00413) | VSLRNLRGYYNQSEAGSHIIQRMYGCDVGPDGR | |
| * Predicted signal | LLRGYDQYAYDGKDYIALNEDLRSWTAADTAA | |
| peptide underlined | QITQRKWEAAREAEQLRAYLEGLCVEWLRRYLK | |
| | NGKETLQRAEHPKTHVTHHPVSDHEATLRCWAL | |
| | GFYPAEITLTWQWDGEDQTQDTELVETRPAGDG | |
| | TFQKWAAVVVPSGEEQRYTCHVQHEGLPEPLTL | |
| | RWEPSSQPTIPIVGIVAGLAVLAVLAVLGAVVAV | |
| | VMCRRKSSGGKGGSCSQAASSNSAQGSDESLIAC | |
| | KA | |
| C*03:03 | MRVMAPRTLILLLSGALALTETWAGSHSMRYFY | (SEQ ID NO: |
| (IMGT/HLA | TAVSRPGRGEPHFIAVGYVDDTQFVRFDSDAASP | 760) |
| Accession No. | RGEPRAPWVEQEGPEYWDRETQKYKRQAQTDR | |
| HLA00411) | VSLRNLRGYYNQSEARSHIIQRMYGCDVGPDGR | |
| * Predicted signal | LLRGYDQYAYDGKDYIALNEDLRSWTAADTAA | |
| peptide underlined | QITQRKWEAAREAEQLRAYLEGLCVEWLRRYLK | |
| | NGKETLQRAEHPKTHVTHHPVSDHEATLRCWAL | |
| | GFYPAEITLTWQWDGEDQTQDTELVETRPAGDG | |
| | TFQKWAAVVVPSGEEQRYTCHVQHEGLPEPLTL | |
| | RWEPSSQPTIPIVGIVAGLAVLAVLAVLGAVVAV | |
| | VMCRRKSSGGKGGSCSQAASSNSAQGSDESLIAC | |
| | KA | |
| C*02:02 | MRVMAPRTLLLLLSGALALTETWACSHSMRYFY | (SEQ ID NO: |
| (IMGT/HLA | TAVSRPSRGEPHFIAVGYVDDTQFVRFDSDAASP | 761) |
| Accession No. | RGEPRAPWVEQEGPEYWDRETQKYKRQAQTDR | |
| HLA00404) | VNLRKLRGYYNQSEAGSHTLQRMYGCDLGPDG | |
| * Predicted signal | RLLRGYDQSAYDGKDYIALNEDLRSWTAADTA | |
| peptide underlined | AQITQRKWEAAREAEQWRAYLEGECVEWLRRY | |
| | LENGKETLQRAEHPKTHVTHHPVSDHEATLRCW | |
| | ALGFYPTEITLTWQRDGEDQTQDTELVETRPAGD | |
| | GTFQKWAAVVVPSGEEQRYTCHVQHEGLPEPLT | |
| | LRWEPSSQPTIPIVGIVAGLAVLAVLAVLGAVVA | |
| | VVMCRRKSSGGKGGSCSQAASSNSAQGSDESLI | |
| | ACKA | |
| C*16:01 | MRVMAPRTLILLLSGALALTETWACSHSMRYFY | (SEQ ID NO: |
| (IMGT/HLA | TAVSRPGRGEPRFIAVGYVDDTQFVRFDSDAASP | 762) |
| Accession No. | RGEPRAPWVEQEGPEYWDRETQKYKRQAQTDR | |
| HLA00475) | VSLRNLRGYYNQSEAGSHTLQWMYGCDLGPDG | |
| * Predicted signal | RLLRGYDQSAYDGKDYIALNEDLRSWTAADTA | |
| peptide underlined | AQITQRKWEAARAAEQQRAYLEGTCVEWLRRY | |
| | LENGKETLQRAEHPKTHVTHHLVSDHEATLRCW | |
| | ALGFYPAEITLTWQRDGEDQTQDTELVETRPAG | |
| | DGTFQKWAAVVVPSGEEQRYTCHVQHEGLPEPL | |
| | TLRWEPSSQPTIPIVGIVAGLAVLAVLAVLGAVV | |
| | AVVMCRRKSSGGKGGSCSQAASSNSAQGSDESL | |
| | IACKA | |
| C*08:02 | MRVMAPRTLILLLSGALALTETWACSHSMRYFY | (SEQ ID NO: |
| (IMGT/HLA | TAVSRPGRGEPRFIAVGYVDDTQFVQFDSDAASP | 763) |
| Accession No. | RGEPRAPWVEQEGPEYWDRETQKYKRQAQTDR | |
| HLA00446) | VSLRNLRGYYNQSEAGSHTLQRMYGCDLGPDG | |
| * Predicted signal | RLLRGYNQFAYDGKDYIALNEDLRSWTAADKA | |
| peptide underlined | AQITQRKWEAAREAEQRRAYLEGTCVEWLRRY | |
| | LENGKKTLQRAEHPKTHVTHHPVSDHEATLRCW | |
| | ALGFYPAEITLTWQRDGEDQTQDTELVETRPAG | |
| | DGTFQKWAAVVVPSGEEQRYTCHVQHEGLPEPL | |
| | TLRWGPSSQPTIPIVGIVAGLAVLAVLAVLGAVM | |
| | AVVMCRRKSSGGKGGSCSQAASSNSAQGSDESL | |
| | IACKA | |
| C*12:03 | MRVMAPRTLILLLSGALALTETWACSHSMRYFY | (SEQ ID NO: |
| (IMGT/HLA | TAVSRPGRGEPRFIAVGYVDDTQFVRFDSDAASP | 764) |
| Accession No. | RGEPRAPWVEQEGPEYWDRETQKYKRQAQADR | |
| HLA00455) | VSLRNLRGYYNQSEAGSHTLQWMYGCDLGPDG | |
| * Predicted signal | RLLRGYDQSAYDGKDYIALNEDLRSWTAADTA | |
| peptide underlined | AQITQRKWEAAREAEQWRAYLEGTCVEWLRRY | |
| | LENGKETLQRAEHPKTHVTHHPVSDHEATLRCW | |
| | ALGFYPAEITLTWQRDGEDQTQDTELVETRPAG | |
| | DGTFQKWAAVVVPSGEEQRYTCHVQHEGLPEPL | |
| | TLRWEPSSQPTIPIVGIVAGLAVLAVLAVLGAVM | |
| | AVVMCRRKSSGGKGGSCSQAASSNSAQGSDESL | |
| | IACKA | |
| C*01:02 | MRVMAPRTLILLLSGALALTETWACSHSMKYFF | (SEQ ID NO: |
| (IMGT/HLA | TSVSRPGRGEPRFISVGYVDDTQFVRFDSDAASP | 765) |
| Accession No. | RGEPRAPWVEQEGPEYWDRETQKYKRQAQTDR | |
| HLA00401) | VSLRNLRGYYNQSEAGSHTLQWMCGCDLGPDG | |
| * Predicted signal | RLLRGYDQYAYDGKDYIALNEDLRSWTAADTA | |
| peptide underlined | AQITQRKWEAAREAEQRRAYLEGTCVEWLRRY | |
| | LENGKETLQRAEHPKTHVTHHPVSDHEATLRCW | |
| | ALGFYPAEITLTWQWDGEDQTQDTELVETRPAG | |
| | DGTFQKWAAVMVPSGEEQRYTCHVQHEGLPEP | |
| | LTLRWEPSSQPTIPIVGIVAGLAVLAVLAVLGAV | |
| | VAVVMCRRKSSGGKGGSCSQAASSNSAQGSDES | |
| | LIACKA | |
| C*15:02 | MRVMAPRTLLLLLSGALALTETWACSHSMRYFY | (SEQ ID NO: |
| (IMGT/HLA | TAVSRPGRGEPHFIAVGYVDDTQFVRFDSDAASP | 766) |
| Accession No. | RGEPRAPWVEQEGPEYWDRETQNYKRQAQTDR | |
| HLA00467) | VNLRKLRGYYNQSEAGSHIIQRMYGCDLGPDGR | |
| * Predicted signal | LLRGHDQLAYDGKDYIALNEDLRSWTAADTAA | |
| peptide underlined | QITQRKWEAAREAEQLRAYLEGTCVEWLRRYLE | |
| | NGKETLQRAEHPKTHVTHHPVSDHEATLRCWAL | |
| | GFYPAEITLTWQRDGEDQTQDTELVETRPAGDG | |
| | TFQKWAAVVVPSGEEQRYTCHVQHEGLPEPLTL | |
| | RWEPSSQPTIPIVGIVAGLAVLAVLAVLGAVMAV | |
| | VMCRRKSSGGKGGSCSQAASSNSAQGSDESLIAC | |
| | KA | |
| C*07:04 | MRVMAPRALLLLLSGGLALTETWACSHSMRYF | (SEQ ID NO: |
| (IMGT/HLA | DTAVSRPGRGEPRFISVGYVDDTQFVRFDSDAAS | 767) |
| Accession No. | PRGEPRAPWVEQEGPEYWDRETQKYKRQAQAD | |
| HLA00406) | RVSLRNLRGYYNQSEDGSHTFQRMYGCDLGPDG | |
| * Predicted signal | RLLRGYDQFAYDGKDYIALNEDLRSWTAADTA | |
| peptide underlined | AQITQRKLEAARAAEQDRAYLEGTCVEWLRRYL | |
| | ENGKKTLQRAEPPKTHVTHHPLSDHEATLRCWA | |
| | LGFYPAEITLTWQRDGEDQTQDTELVETRPAGD | |
| | GTFQKWAAVVVPSGQEQRYTCHMQHEGLQEPL | |
| | TLSWEPSSQPTIPIMGIVAGLAVLVVLAVLGAVV | |
| | TAMMCRRKSSGGKGGSCSQAACSNSAQGSDESL | |
| | ITCKA | |
| C*14:02 | MRVMAPRTLILLLSGALALTETWACSHSMRYFS | (SEQ ID NO: |
| (IMGT/HLA | TSVSRPGRGEPRFIAVGYVDDTQFVRFDSDAASP | 768) |
| Accession No. | RGEPRAPWVEQEGPEYWDRETQKYKRQAQTDR | |
| HLA00462) | VSLRNLRGYYNQSEAGSHTLQWMFGCDLGPDG | |
| * Predicted signal | RLLRGYDQSAYDGKDYIALNEDLRSWTAADTA | |
| peptide underlined | AQITQRKWEAAREAEQRRAYLEGTCVEWLRRY | |
| | LENGKETLQRAEHPKTHVTHHPVSDHEATLRCW | |
| | ALGFYPAEITLTWQWDGEDQTQDTELVETRPAG | |
| | DGTFQKWAAVVVPSGEEQRYTCHVQHEGLPEPL | |
| | TLRWEPSSQPTIPIVGIVAGLAVLAVLAVLGAVV | |
| | AVVMCRRKSSGGKGGSCSQAASSNSAQGSDESL | |
| | IACKA | |
| B*08:01 | MLVMAPRTVLLLLSAALALTETWAGSHSMRYF | (SEQ ID NO: |
| (IMGT/HLA | DTAMSRPGRGEPRFISVGYVDDTQFVRFDSDAAS | 769) |
| Accession No. | PREEPRAPWIEQEGPEYWDRNTQIFKTNTQTDRE | |
| HLA00146) | SLRNLRGYYNQSEAGSHTLQSMYGCDVGPDGRL | |
| * Predicted signal | LRGHNQYAYDGKDYIALNEDLRSWTAADTAAQI | |
| peptide underlined | TQRKWEAARVAEQDRAYLEGTCVEWLRRYLEN | |
| | GKDTLERADPPKTHVTHHPISDHEATLRCWALG | |
| | FYPAEITLTWQRDGEDQTQDTELVETRPAGDRTF | |
| | QKWAAVVVPSGEEQRYTCHVQHEGLPKPLTLR | |
| | WEPSSQSTVPIVGIVAGLAVLAVVVIGAVVAAV | |
| | MCRRKSSGGKGGSYSQAACSDSAQGSDVSLTA | |
| B*07:02 | MLVMAPRTVLLLLSAALALTETWAGSHSMRYF | (SEQ ID NO: |
| (IMGT/HLA | YTSVSRPGRGEPRFISVGYVDDTQFVRFDSDAAS | 770) |
| Accession No. | PREEPRAPWIEQEGPEYWDRNTQIYKAQAQTDR | |
| HLA00132) | ESLRNLRGYYNQSEAGSHTLQSMYGCDVGPDGR | |
| * Predicted signal | LLRGHDQYAYDGKDYIALNEDLRSWTAADTAA | |
| peptide underlined | QITQRKWEAAREAEQRRAYLEGECVEWLRRYLE | |
| | NGKDKLERADPPKTHVTHHPISDHEATLRCWAL | |
| | GFYPAEITLTWQRDGEDQTQDTELVETRPAGDR | |
| | TFQKWAAVVVPSGEEQRYTCHVQHEGLPKPLTL | |
| | RWEPSSQSTVPIVGIVAGLAVLAVVVIGAVVAAV | |
| | MCRRKSSGGKGGSYSQAACSDSAQGSDVSLTA | |
| B*44:02 | MRVTAPRTLLLLLWGAVALTETWAGSHSMRYF | (SEQ ID NO: |
| (IMGT/HLA | YTAMSRPGRGEPRFITVGYVDDTLFVRFDSDATS | 771) |
| Accession No. | PRKEPRAPWIEQEGPEYWDRETQISKTNTQTYRE | |
| HLA00318) | NLRTALRYYNQSEAGSHIIQRMYGCDVGPDGRL | |
| * Predicted signal | LRGYDQDAYDGKDYIALNEDLSSWTAADTAAQI | |
| peptide underlined | TQRKWEAARVAEQDRAYLEGLCVESLRRYLEN | |
| | GKETLQRADPPKTHVTHHPISDHEVTLRCWALG | |
| | FYPAEITLTWQRDGEDQTQDTELVETRPAGDRTF | |
| | QKWAAVVVPSGEEQRYTCHVQHEGLPKPLTLR | |
| | WEPSSQSTVPIVGIVAGLAVLAVVVIGAVVAAV | |
| | MCRRKSSGGKGGSYSQAACSDSAQGSDVSLTA | |
| B*15:01 | MRVTAPRTVLLLLSGALALTETWAGSHSMRYFY | (SEQ ID NO: |
| (IMGT/HLA | TAMSRPGRGEPRFIAVGYVDDTQFVRFDSDAASP | 772) |
| Accession No. | RMAPRAPWIEQEGPEYWDRETQISKTNTQTYRES | |
| HLA00162) | LRNLRGYYNQSEAGSHTLQRMYGCDVGPDGRL | |
| * Predicted signal | LRGHDQSAYDGKDYIALNEDLSSWTAADTAAQI | |
| peptide underlined | TQRKWEAAREAEQWRAYLEGLCVEWLRRYLEN | |
| | GKETLQRADPPKTHVTHHPISDHEATLRCWALG | |
| | FYPAEITLTWQRDGEDQTQDTELVETRPAGDRTF | |
| | QKWAAVVVPSGEEQRYTCHVQHEGLPKPLTLR | |
| | WEPSSQSTIPIVGIVAGLAVLAVVVIGAVVATVM | |
| | CRRKSSGGKGGSYSQAASSDSAQGSDVSLTA | |
| B*40:01 | MRVTAPRTVLLLLSAALALTETWAGSHSMRYFH | (SEQ ID NO: |
| (IMGT/HLA | TAMSRPGRGEPRFITVGYVDDTLFVRFDSDATSP | 773) |
| Accession No. | RKEPRAPWIEQEGPEYWDRETQISKTNTQTYRES | |
| HLA00291) | LRNLRGYYNQSEAGSHTLQRMYGCDVGPDGRL | |
| * Predicted signal | LRGHNQYAYDGKDYIALNEDLRSWTAADTAAQI | |
| peptide underlined | SQRKLEAARVAEQLRAYLEGECVEWLRRYLENG | |
| | KDKLERADPPKTHVTHHPISDHEATLRCWALGF | |
| | YPAEITLTWQRDGEDQTQDTELVETRPAGDRTF | |
| | QKWAAVVVPSGEEQRYTCHVQHEGLPKPLTLR | |
| | WEPSSQSTVPIVGIVAGLAVLAVVVIGAVVAAV | |
| | MCRRKSSGGKGGSYSQAACSDSAQGSDVSLTA | |
| B*44:03 | MRVTAPRTLLLLLWGAVALTETWAGSHSMRYF | (SEQ ID NO: |
| (IMGT/HLA | YTAMSRPGRGEPRFITVGYVDDTLFVRFDSDATS | 774) |
| Accession No. | PRKEPRAPWIEQEGPEYWDRETQISKTNTQTYRE | |
| HLA00319) | NLRTALRYYNQSEAGSHIIQRMYGCDVGPDGRL | |
| * Predicted signal | LRGYDQDAYDGKDYIALNEDLSSWTAADTAAQI | |
| peptide underlined | TQRKWEAARVAEQLRAYLEGLCVESLRRYLENG | |
| | KETLQRADPPKTHVTHHPISDHEVTLRCWALGF | |
| | YPAEITLTWQRDGEDQTQDTELVETRPAGDRTF | |
| | QKWAAVVVPSGEEQRYTCHVQHEGLPKPLTLR | |
| | WEPSSQSTVPIVGIVAGLAVLAVVVIGAVVAAV | |
| | MCRRKSSGGKGGSYSQAACSDSAQGSDVSLTA | |
| B*35:01 | MRVTAPRTVLLLLWGAVALTETWAGSHSMRYF | (SEQ ID NO: |
| (IMGT/HLA | YTAMSRPGRGEPRFIAVGYVDDTQFVRFDSDAA | 775) |
| Accession No. | SPRTEPRAPWIEQEGPEYWDRNTQIFKTNTQTYR | |
| HLA00237) | ESLRNLRGYYNQSEAGSHIIQRMYGCDLGPDGRL | |
| * Predicted signal | LRGHDQSAYDGKDYIALNEDLSSWTAADTAAQI | |
| peptide underlined | TQRKWEAARVAEQLRAYLEGLCVEWLRRYLEN | |
| | GKETLQRADPPKTHVTHHPVSDHEATLRCWALG | |
| | FYPAEITLTWQRDGEDQTQDTELVETRPAGDRTF | |
| | QKWAAVVVPSGEEQRYTCHVQHEGLPKPLTLR | |
| | WEPSSQSTIPIVGIVAGLAVLAVVVIGAVVATVM | |
| | CRRKSSGGKGGSYSQAASSDSAQGSDVSLTA | |
| B*51:01 | MRVTAPRTVLLLLWGAVALTETWAGSHSMRYF | (SEQ ID NO: |
| (IMGT/HLA | YTAMSRPGRGEPRFIAVGYVDDTQFVRFDSDAA | 776) |
| Accession No. | SPRTEPRAPWIEQEGPEYWDRNTQIFKTNTQTYR | |
| HLA00344) | ENLRIALRYYNQSEAGSHTWQTMYGCDVGPDG | |
| * Predicted signal | RLLRGHNQYAYDGKDYIALNEDLSSWTAADTA | |
| peptide underlined | AQITQRKWEAAREAEQLRAYLEGLCVEWLRRHL | |
| | ENGKETLQRADPPKTHVTHHPVSDHEATLRCWA | |
| | LGFYPAEITLTWQRDGEDQTQDTELVETRPAGD | |
| | RTFQKWAAVVVPSGEEQRYTCHVQHEGLPKPLT | |
| | LRWEPSSQSTIPIVGIVAGLAVLAVVVIGAVVAT | |
| | VMCRRKSSGGKGGSYSQAASSDSAQGSDVSLTA | |
| B*27:05 | MRVTAPRTLLLLLWGAVALTETWAGSHSMRYF | (SEQ ID NO: |
| (IMGT/HLA | HTSVSRPGRGEPRFITVGYVDDTLFVRFDSDAAS | 777) |
| Accession No. | PREEPRAPWIEQEGPEYWDRETQICKAKAQTDRE | |
| HLA00225) | DLRTLLRYYNQSEAGSHTLQNMYGCDVGPDGR | |
| * Predicted signal | LLRGYHQDAYDGKDYIALNEDLSSWTAADTAA | |
| peptide underlined | QITQRKWEAARVAEQLRAYLEGECVEWLRRYLE | |
| | NGKETLQRADPPKTHVTHHPISDHEATLRCWAL | |
| | GFYPAEITLTWQRDGEDQTQDTELVETRPAGDR | |
| | TFQKWAAVVVPSGEEQRYTCHVQHEGLPKPLTL | |
| | RWEPSSQSTVPIVGIVAGLAVLAVVVIGAVVAAV | |
| | MCRRKSSGGKGGSYSQAACSDSAQGSDVSLTA | |
| B*57:01 | MRVTAPRTVLLLLWGAVALTETWAGSHSMRYF | (SEQ ID NO: |
| (IMGT/HLA | YTAMSRPGRGEPRFIAVGYVDDTQFVRFDSDAA | 778) |
| Accession No. | SPRMAPRAPWIEQEGPEYWDGETRNMKASAQT | |
| HLA00381) | YRENLRIALRYYNQSEAGSHIIQVMYGCDVGPD | |
| * Predicted signal | GRLLRGHDQSAYDGKDYIALNEDLSSWTAADTA | |
| peptide underlined | AQITQRKWEAARVAEQLRAYLEGLCVEWLRRY | |
| | LENGKETLQRADPPKTHVTHHPISDHEATLRCW | |
| | ALGFYPAEITLTWQRDGEDQTQDTELVETRPAG | |
| | DRTFQKWAAVVVPSGEEQRYTCHVQHEGLPKPL | |
| | TLRWEPSSQSTVPIVGIVAGLAVLAVVVIGAVVA | |
| | AVMCRRKSSGGKGGSYSQAACSDSAQGSDVSLTA | |
| B*18:01 | MRVTAPRTLLLLLWGAVALTETWAGSHSMRYF | (SEQ ID NO: |
| (IMGT/HLA | HTSVSRPGRGEPRFISVGYVDGTQFVRFDSDAAS | 779) |
| Accession No. | PRTEPRAPWIEQEGPEYWDRNTQISKTNTQTYRE | |
| HLA00213) | SLRNLRGYYNQSEAGSHTLQRMYGCDVGPDGR | |
| * Predicted signal | LLRGHDQSAYDGKDYIALNEDLSSWTAADTAAQ | |
| peptide underlined | ITQRKWEAARVAEQLRAYLEGTCVEWLRRHLEN | |
| | GKETLQRADPPKTHVTHHPISDHEATLRCWALG | |
| | FYPAEITLTWQRDGEDQTQDTELVETRPAGDRTF | |
| | QKWAAVVVPSGEEQRYTCHVQHEGLPKPLTLR | |
| | WEPSSQSTIPIVGIVAGLAVLAVVVIGAVVATVM | |
| | CRRKSSGGKGGSYSQAASSDSAQGSDVSLTA | |
| B*14:02 | MLVMAPRTVLLLLSAALALTETWAGSHSMRYF | (SEQ ID NO: |
| (IMGT/HLA | YTAVSRPGRGEPRFISVGYVDDTQFVRFDSDAAS | 780) |
| Accession No. | PREEPRAPWIEQEGPEYWDRNTQICKTNTQTDRE | |
| HLA00158) | SLRNLRGYYNQSEAGSHTLQWMYGCDVGPDGR | |
| * Predicted signal | LLRGYNQFAYDGKDYIALNEDLSSWTAADTAAQ | |
| peptide underlined | ITQRKWEAAREAEQLRAYLEGTCVEWLRRHLEN | |
| | GKETLQRADPPKTHVTHHPISDHEATLRCWALG | |
| | FYPAEITLTWQRDGEDQTQDTELVETRPAGDRTF | |
| | QKWAAVVVPSGEEQRYTCHVQHEGLPKPLTLR | |
| | WEPSSQSTVPIVGIVAGLAVLAVVVIGAVVAAV | |
| | MCRRKSSGGKGGSYSQAASSDSAQGSDVSLTA | |
| B*13:02 | MRVTAPRTLLLLLWGAVALTETWAGSHSMRYF | (SEQ ID NO: |
| (IMGT/HLA | YTAMSRPGRGEPRFITVGYVDDTQFVRFDSDATS | 781) |
| Accession No. | PRMAPRAPWIEQEGPEYWDRETQISKTNTQTYRE | |
| HLA00153) | NLRTALRYYNQSEAGSHTWQTMYGCDLGPDGR | |
| * Predicted signal | LLRGHNQLAYDGKDYIALNEDLSSWTAADTAA | |
| peptide underlined | QITQLKWEAARVAEQLRAYLEGECVEWLRRYLE | |
| | NGKETLQRADPPKTHVTHHPISDHEATLRCWAL | |
| | GFYPAEITLTWQRDGEDQTQDTELVETRPAGDR | |
| | TFQKWAAVVVPSGEEQRYTCHVQHEGLPKPLTL | |
| | RWEPSSQSTVPIVGIVAGLAVLAVVVIGAVVAAV | |
| | MCRRKSSGGKGGSYSQAACSDSAQGSDVSLTA | |
| B*55:01 | MRVTAPRTLLLLLWGALALTETWAGSHSMRYF | (SEQ ID NO: |
| (IMGT/HLA | YTAMSRPGRGEPRFIAVGYVDDTQFVRFDSDAA | 782) |
| Accession No. | SPREEPRAPWIEQEGPEYWDRNTQIYKAQAQTD | |
| HLA00368) | RESLRNLRGYYNQSEAGSHTWQTMYGCDLGPD | |
| * Predicted signal | GRLLRGHNQLAYDGKDYIALNEDLSSWTAADTA | |
| peptide underlined | AQITQRKWEAAREAEQLRAYLEGTCVEWLRRYL | |
| | ENGKETLQRADPPKTHVTHHPISDHEATLRCWA | |
| | LGFYPAEITLTWQRDGEDQTQDTELVETRPAGD | |
| | RTFQKWAAVVVPSGEEQRYTCHVQHEGLPKPLT | |
| | LRWEPSSQSTIPIVGIVAGLAVLAVVVIGAVVAT | |
| | VMCRRKSSGGKGGSYSQAASSDSAQGSDVSLTA | |
| B*14:01 | MLVMAPRTVLLLLSAALALTETWAGSHSMRYF | (SEQ ID NO: |
| (IMGT/HLA | YTSVSRPGRGEPRFISVGYVDDTQFVRFDSDAAS | 783) |
| Accession No. | PREEPRAPWIEQEGPEYWDRNTQICKTNTQTDRE | |
| HLA00157) | SLRNLRGYYNQSEAGSHTLQWMYGCDVGPDGR | |
| * Predicted signal | LLRGYNQFAYDGKDYIALNEDLSSWTAADTAAQ | |
| peptide underlined | ITQRKWEAAREAEQLRAYLEGTCVEWLRRHLEN | |
| | GKETLQRADPPKTHVTHHPISDHEATLRCWALG | |
| | FYPAEITLTWQRDGEDQTQDTELVETRPAGDRTF | |
| | QKWAAVVVPSGEEQRYTCHVQHEGLPKPLTLR | |
| | WEPSSQSTVPIVGIVAGLAVLAVVVIGAVVAAV | |
| | MCRRKSSGGKGGSYSQAASSDSAQGSDVSLTA | |
| B*49:01 | MRVTAPRTVLLLLSAALALTETWAGSHSMRYFH | (SEQ ID NO: |
| (IMGT/HLA | TAMSRPGRGEPRFITVGYVDDTLFVRFDSDATSP | 784) |
| Accession No. | RKEPRAPWIEQEGPEYWDRETQISKTNTQTYREN | |
| HLA00340) | LRIALRYYNQSEAGSHTWQRMYGCDLGPDGRLL | |
| * Predicted signal | RGYNQLAYDGKDYIALNEDLSSWTAADTAAQIT | |
| peptide underlined | QRKWEAAREAEQLRAYLEGLCVEWLRRYLENG | |
| | KETLQRADPPKTHVTHHPISDHEATLRCWALGF | |
| | YPAEITLTWQRDGEDQTQDTELVETRPAGDRTF | |
| | QKWAAVVVPSGEEQRYTCHVQHEGLPKPLTLR | |
| | WEPSSQSTIPIVGIVAGLAVLAVVVIGAVVATVM | |
| | CRRKSSGGKGGSYSQAASSDSAQGSDVSLTA | |
| B*37:01 | MRVTAPRTLLLLLWGAVALTETWAGSHSMRYF | (SEQ ID NO: |
| (IMGT/HLA | HTSVSRPGRGEPRFISVGYVDDTQFVRFDSDAAS | 785) |
| Accession No. | PRTEPRAPWIEQEGPEYWDRETQISKTNTQTYRE | |
| HLA00265) | DLRTLLRYYNQSEAGSHTIQRMSGCDVGPDGRL | |
| * Predicted signal | LRGYNQFAYDGKDYIALNEDLSSWTAADTAAQI | |
| peptide underlined | TQRKWEAARVAEQDRAYLEGTCVEWLRRYLEN | |
| | GKETLQRADPPKTHVTHHPISDHEATLRCWALG | |
| | FYPAEITLTWQRDGEDQTQDTELVETRPAGDRTF | |
| | QKWAAVVVPSGEEQRYTCHVQHEGLPKPLTLR | |
| | WEPSSQSTIPIVGIVAGLAVLAVVVIGAVVATVM | |
| | CRRKSSGGKGGSYSQAASSDSAQGSDVSLTA | |
| B*38:01 | MLVMAPRTVLLLLSAALALTETWAGSHSMRYF | (SEQ ID NO: |
| (IMGT/HLA | YTSVSRPGRGEPRFISVGYVDDTQFVRFDSDAAS | 786) |
| Accession No. | PREEPRAPWIEQEGPEYWDRNTQICKTNTQTYRE | |
| HLA00267) | NLRIALRYYNQSEAGSHTLQRMYGCDVGPDGRL | |
| * Predicted signal | LRGHNQFAYDGKDYIALNEDLSSWTAADTAAQI | |
| peptide underlined | TQRKWEAARVAEQLRTYLEGTCVEWLRRYLEN | |
| | GKETLQRADPPKTHVTHHPISDHEATLRCWALG | |
| | FYPAEITLTWQRDGEDQTQDTELVETRPAGDRTF | |
| | QKWAAVVVPSGEEQRYTCHVQHEGLPKPLTLR | |
| | WEPSSQSTVPIVGIVAGLAVLAVVVIGAVVAAV | |
| | MCRRKSSGGKGGSYSQAASSDSAQGSDVSLTA | |
| B*39:01 | MLVMAPRTVLLLLSAALALTETWAGSHSMRYF | (SEQ ID NO: |
| (IMGT/HLA | YTSVSRPGRGEPRFISVGYVDDTQFVRFDSDAAS | 787) |
| Accession No. | PREEPRAPWIEQEGPEYWDRNTQICKTNTQTDRE | |
| HLA00271) | SLRNLRGYYNQSEAGSHTLQRMYGCDVGPDGR | |
| * Predicted signal | LLRGHNQFAYDGKDYIALNEDLSSWTAADTAAQ | |
| peptide underlined | ITQRKWEAARVAEQLRTYLEGTCVEWLRRYLEN | |
| | GKETLQRADPPKTHVTHHPISDHEATLRCWALG | |
| | FYPAEITLTWQRDGEDQTQDTELVETRPAGDRTF | |
| | QKWAAVVVPSGEEQRYTCHVQHEGLPKPLTLR | |
| | WEPSSQSTVPIVGIVAGLAVLAVVVIGAVVAAV | |
| | MCRRKSSGGKGGSYSQAASSDSAQGSDVSLTA | |
| B*35:03 | MRVTAPRTVLLLLWGAVALTETWAGSHSMRYF | (SEQ ID NO: |
| (IMGT/HLA | YTAMSRPGRGEPRFIAVGYVDDTQFVRFDSDAA | 788) |
| Accession No. | SPRTEPRAPWIEQEGPEYWDRNTQIFKTNTQTYR | |
| HLA00239) | ESLRNLRGYYNQSEAGSHIIQRMYGCDLGPDGRL | |
| * Predicted signal | LRGHDQFAYDGKDYIALNEDLSSWTAADTAAQI | |
| peptide underlined | TQRKWEAARVAEQLRAYLEGLCVEWLRRYLEN | |
| | GKETLQRADPPKTHVTHHPVSDHEATLRCWALG | |
| | FYPAEITLTWQRDGEDQTQDTELVETRPAGDRTF | |
| | QKWAAVVVPSGEEQRYTCHVQHEGLPKPLTLR | |
| | WEPSSQSTIPIVGIVAGLAVLAVVVIGAVVATVM | |
| | CRRKSSGGKGGSYSQAASSDSAQGSDVSLTA | |
| B*40:02 | MRVTAPRTLLLLLWGAVALTETWAGSHSMRYF | (SEQ ID NO: |
| (IMGT/HLA | HTSVSRPGRGEPRFITVGYVDDTLFVRFDSDATSP | 789) |
| Accession No. | RKEPRAPWIEQEGPEYWDRETQISKTNTQTYRES | |
| HLA00293) | LRNLRGYYNQSEAGSHTLQSMYGCDVGPDGRLL | |
| * Predicted signal | RGHNQYAYDGKDYIALNEDLRSWTAADTAAQIT | |
| peptide underlined | QRKWEAARVAEQLRAYLEGECVEWLRRYLENG | |
| | KETLQRADPPKTHVTHHPISDHEATLRCWALGF | |
| | YPAEITLTWQRDGEDQTQDTELVETRPAGDRTF | |
| | QKWAAVVVPSGEEQRYTCHVQHEGLPKPLTLR | |
| | WEPSSQSTVPIVGIVAGLAVLAVVVIGAVVAAV | |
| | MCRRKSSGGKGGSYSQAACSDSAQGSDVSLTA | |
| DRB1*07:01 | MVCLKLPGGSCMAALTVTLMVLSSPLALAGDTQ | (SEQ ID NO: |
| (IMGT/HLA | PRFLWQGKYKCHFFNGTERVQFLERLFYNQEEF | 790) |
| Accession No. | VRFDSDVGEYRAVTELGRPVAESWNSQKDILED | |
| HLA00719) | RRGQVDTVCRHNYGVGESFTVQRRVHPEVTVYP | |
| * Predicted signal | AKTQPLQHHNLLVCSVSGFYPGSIEVRWFRNGQ | |
| peptide underlined | EEKAGVVSTGLIQNGDWTFQTLVMLETVPRSGE | |
| | VYTCQVEHPSVMSPLTVEWRARSESAQSKMLSG | |
| | VGGFVLGLLFLGAGLFIYFRNQKGHSGLQPTGFLS | |
| DRB1*03:01 | MVCLRLPGGSCMAVLTVTLMVLSSPLALAGDTR | (SEQ ID NO: |
| (IMGT/HLA | PRFLEYSTSECHFFNGTERVRYLDRYFHNQEENV | 791) |
| Accession No. | RFDSDVGEFRAVTELGRPDAEYWNSQKDLLEQK | |
| HLA00671) | RGRVDNYCRHNYGVVESFTVQRRVHPKVTVYPS | |
| * Predicted signal | KTQPLQHHNLLVCSVSGFYPGSIEVRWFRNGQEE | |
| peptide underlined | KTGVVSTGLIHNGDWTFQTLVMLETVPRSGEVY | |
| | TCQVEHPSVTSPLTVEWRARSESAQSKMLSGVG | |
| | GFVLGLLFLGAGLFIYFRNQKGHSGLQPRGFLS | |
| DRB1*15:01 | MVCLKLPGGSCMTALTVTLMVLSSPLALSGDTR | (SEQ ID NO: |
| (IMGT/HLA | PRFLWQPKRECHFFNGTERVRFLDRYFYNQEESV | 792) |
| Accession No. | RFDSDVGEFRAVTELGRPDAEYWNSQKDILEQA | |
| HLA00865) | RAAVDTYCRHNYGVVESFTVQRRVQPKVTVYPS | |
| * Predicted signal | KTQPLQHHNLLVCSVSGFYPGSIEVRWFLNGQEE | |
| peptide underlined | KAGMVSTGLIQNGDWTFQTLVMLETVPRSGEVY | |
| | TCQVEHPSVTSPLTVEWRARSESAQSKMLSGVG | |
| | GFVLGLLFLGAGLFIYFRNQKGHSGLQPTGFLS | |
| DRB1*04:01 | MVCLKFPGGSCMAALTVTLMVLSSPLALAGDTR | (SEQ ID NO: |
| (IMGT/HLA | PRFLEQVKHECHFFNGTERVRFLDRYFYHQEEY | 793) |
| Accession No. | VRFDSDVGEYRAVTELGRPDAEYWNSQKDLLEQ | |
| HLA00685) | KRAAVDTYCRHNYGVGESFTVQRRVYPEVTVYP | |
| * Predicted signal | AKTQPLQHHNLLVCSVNGFYPGSIEVRWFRNGQ | |
| peptide underlined | EEKTGVVSTGLIQNGDWTFQTLVMLETVPRSGE | |
| | VYTCQVEHPSLTSPLTVEWRARSESAQSKMLSG | |
| | VGGFVLGLLFLGAGLFIYFRNQKGHSGLQPTGFLS | |
| DRB1*01:01 | MVCLKLPGGSCMTALTVTLMVLSSPLALAGDTR | (SEQ ID NO: |
| (IMGT/HLA | PRFLWQLKFECHFFNGTERVRLLERCIYNQEESV | 794) |
| Accession No. | RFDSDVGEYRAVTELGRPDAEYWNSQKDLLEQR | |
| HLA00664) | RAAVDTYCRHNYGVGESFTVQRRVEPKVTVYPS | |
| * Predicted signal | KTQPLQHHNLLVCSVSGFYPGSIEVRWFRNGQEE | |
| peptide underlined | KAGVVSTGLIQNGDWTFQTLVMLETVPRSGEVY | |
| | TCQVEHPSVTSPLTVEWRARSESAQSKMLSGVG | |
| | GFVLGLLFLGAGLFIYFRNQKGHSGLQPTGFLS | |
| DRB1*13:01 | MVCLRLPGGSCMAVLTVTLMVLSSPLALAGDTR | (SEQ ID NO: |
| (IMGT/HLA | PRFLEYSTSECHFFNGTERVRFLDRYFHNQEENV | 795) |
| Accession No. | RFDSDVGEFRAVTELGRPDAEYWNSQKDILEDE | |
| HLA00797) | RAAVDTYCRHNYGVVESFTVQRRVHPKVTVYPS | |
| * Predicted signal | KTQPLQHHNLLVCSVSGFYPGSIEVRWFRNGQEE | |
| peptide underlined | KTGVVSTGLIHNGDWTFQTLVMLETVPRSGEVY | |
| | TCQVEHPSVTSPLTVEWRARSESAQSKMLSGVG | |
| | GFVLGLLFLGAGLFIYFRNQKGHSGLQPRGFLS | |
| DRB1*11:01 | MVCLRLPGGSCMAVLTVTLMVLSSPLALAGDTR | (SEQ ID NO: |
| (IMGT/HLA | PRFLEYSTSECHFFNGTERVRFLDRYFYNQEEYV | 796) |
| Accession No. | RFDSDVGEFRAVTELGRPDEEYWNSQKDFLEDR | |
| HLA00751) | RAAVDTYCRHNYGVGESFTVQRRVHPKVTVYPS | |
| * Predicted signal | KTQPLQHHNLLVCSVSGFYPGSIEVRWFRNGQEE | |
| peptide underlined | KTGVVSTGLIHNGDWTFQTLVMLETVPRSGEVY | |
| | TCQVEHPSVTSPLTVEWRARSESAQSKMLSGVG | |
| | GFVLGLLFLGAGLFIYFRNQKGHSGLQPRGFLS | |
| DRB1*04:04 | MVCLKFPGGSCMAALTVTLMVLSSPLALAGDTR | (SEQ ID NO: |
| (IMGT/HLA | PRFLEQVKHECHFFNGTERVRFLDRYFYHQEEY | 797) |
| Accession No. | VRFDSDVGEYRAVTELGRPDAEYWNSQKDLLEQ | |
| HLA00689) | RRAAVDTYCRHNYGVVESFTVQRRVYPEVTVYP | |
| * Predicted signal | AKTQPLQHHNLLVCSVNGFYPGSIEVRWFRNGQ | |
| peptide underlined | EEKTGVVSTGLIQNGDWTFQTLVMLETVPRSGE | |
| | VYTCQVEHPSLTSPLTVEWRARSESAQSKMLSG | |
| | VGGFVLGLLFLGAGLFIYFRNQKGHSGLQPTGFLS | |
| DRB1*13:02 | MVCLRLPGGSCMAVLTVTLMVLSSPLALAGDTR | (SEQ ID NO: |
| (IMGT/HLA | PRFLEYSTSECHFFNGTERVRFLDRYFHNQEENV | 798) |
| Accession No. | RFDSDVGEFRAVTELGRPDAEYWNSQKDILEDE | |
| HLA00798) | RAAVDTYCRHNYGVGESFTVQRRVHPKVTVYPS | |
| * Predicted signal | KTQPLQHHNLLVCSVSGFYPGSIEVRWFRNGQEE | |
| peptide underlined | KTGVVSTGLIHNGDWTFQTLVMLETVPRSGEVY | |
| | TCQVEHPSVTSPLTVEWRARSESAQSKMLSGVG | |
| | GFVLGLLFLGAGLFIYFRNQKGHSGLQPRGFLS | |
| DRB1*08:01 | MVCLRLPGGSCMAVLTVTLMVLSSPLALAGDTR | (SEQ ID NO: |
| (IMGT/HLA | PRFLEYSTGECYFFNGTERVRFLDRYFYNQEEYV | 799) |
| Accession No. | RFDSDVGEYRAVTELGRPSAEYWNSQKDFLEDR | |
| HLA00723) | RALVDTYCRHNYGVGESFTVQRRVHPKVTVYPS | |
| * Predicted signal | KTQPLQHHNLLVCSVSGFYPGSIEVRWFRNGQEE | |
| peptide underlined | KTGVVSTGLIHNGDWTFQTLVMLETVPRSGEVY | |
| | TCQVEHPSVTSPLTVEWSARSESAQSKMLSGVG | |
| | GFVLGLLFLGAGLFIYFRNQKGHSGLQPTGFLS | |
| DRB1*12:01 | MVCLRLPGGSCMAVLTVTLMVLSSPLALAGDTR | (SEQ ID NO: |
| (IMGT/HLA | PRFLEYSTGECYFFNGTERVRLLERHFHNQEELL | 800) |
| Accession No. | RFDSDVGEFRAVTELGRPVAESWNSQKDILEDRR | |
| HLA00789) | AAVDTYCRHNYGAVESFTVQRRVHPKVTVYPSK | |
| * Predicted signal | TQPLQHHNLLVCSVSGFYPGSIEVRWFRNGQEEK | |
| peptide underlined | TGVVSTGLIHNGDWTFQTLVMLETVPRSGEVYT | |
| | CQVEHPSVTSPLTVEWRARSESAQSKMLSGVGG | |
| | FVLGLLFLGAGLFIYFRNQKGHSGLQPRGFLS | |
| DRB1*11:04 | MVCLRLPGGSCMAVLTVTLMVLSSPLALAGDTR | (SEQ ID NO: |
| (IMGT/HLA | PRFLEYSTSECHFFNGTERVRFLDRYFYNQEEYV | 801) |
| Accession No. | RFDSDVGEFRAVTELGRPDEEYWNSQKDFLEDR | |
| HLA00756) | RAAVDTYCRHNYGVVESFTVQRRVHPKVTVYPS | |
| * Predicted signal | KTQPLQHHNLLVCSVSGFYPGSIEVRWFRNGQEE | |
| peptide underlined | KTGVVSTGLIHNGDWTFQTLVMLETVPRSGEVY | |
| | TCQVEHPSVTSPLTVEWRARSESAQSKMLSGVG | |
| | GFVLGLLFLGAGLFIYFRNQKGHSGLQPRGFLS | |
| DRB1*09:01 | MVCLKLPGGSCMAALTVTLMVLSSPLALAGDTQ | (SEQ ID NO: |
| (IMGT/HLA | PRFLKQDKFECHFFNGTERVRYLHRGIYNQEENV | 802) |
| Accession No. | RFDSDVGEYRAVTELGRPVAESWNSQKDFLERR | |
| HLA00749) | RAEVDTVCRHNYGVGESFTVQRRVHPEVTVYPA | |
| * Predicted signal | KTQPLQHHNLLVCSVSGFYPGSIEVRWFRNGQEE | |
| peptide underlined | KAGVVSTGLIQNGDWTFQTLVMLETVPRSGEVY | |
| | TCQVEHPSVMSPLTVEWRARSESAQSKMLSGVG | |
| | GFVLGLLFLGAGLFIYFRNQKGHSGLQPTGFLS | |
| DRB1*14:01 | MVCLRLPGGSCMAVLTVTLMVLSSPLALAGDTR | (SEQ ID NO: |
| (IMGT/HLA | PRFLEYSTSECHFFNGTERVRFLDRYFHNQEEFV | 803) |
| Accession No. | RFDSDVGEYRAVTELGRPAAEHWNSQKDLLERR | |
| HLA00833) | RAEVDTYCRHNYGVVESFTVQRRVHPKVTVYPS | |
| * Predicted signal | KTQPLQHYNLLVCSVSGFYPGSIEVRWFRNGQEE | |
| peptide underlined | KTGVVSTGLIHNGDWTFQTLVMLETVPRSGEVY | |
| | TCQVEHPSVTSPLTVEWRARSESAQSKMLSGVG | |
| | GFVLGLLFLGAGLFIYFRNQKGHSGLQPRGFLS | |
| DRB1*04:07 | MVCLKFPGGSCMAALTVTLMVLSSPLALAGDTR | (SEQ ID NO: |
| (IMGT/HLA | PRFLEQVKHECHFFNGTERVRFLDRYFYHQEEY | 804) |
| Accession No. | VRFDSDVGEYRAVTELGRPDAEYWNSQKDLLEQ | |
| HLA00693) | RRAEVDTYCRHNYGVGESFTVQRRVYPEVTVYP | |
| * Predicted signal | AKTQPLQHHNLLVCSVNGFYPGSIEVRWFRNGQ | |
| peptide underlined | EEKTGVVSTGLIQNGDWTFQTLVMLETVPRSGE | |
| | VYTCQVEHPSLTSPLTVEWRARSESAQSKMLSG | |
| | VGGFVLGLLFLGAGLFIYFRNQKGHSGLQPTGFLS | |
| DRB1*14:04 | MVCLRLPGGSCMAVLTVTLMVLSSPLALAGDTR | (SEQ ID NO: |
| (IMGT/HLA | PRFLEYSTGECYFFNGTERVRFLDRYFHNQEEFV | 805) |
| Accession No. | RFDSDVGEYRAVTELGRPAAEHWNSQKDLLERR | |
| HLA00836) | RAEVDTYCRHNYGVVESFTVQRRVHPKVTVYPS | |
| * Predicted signal | KTQPLQHHNLLVCSVSGFYPGSIEVRWFRNGQEE | |
| peptide underlined | KTGVVSTGLIHNGDWTFQTLVMLETVPRSGEVY | |
| | TCQVEHPSVTSPLTVEWRARSESAQSKMLSGVG | |
| | GFVLGLLFLGAGLFIYFRNQKGHSGLQPRGFLS | |
| DQA1*05:01 | MILNKALMLGALALTTVMSPCGGEDIVADHVAS | (SEQ ID NO: |
| (IMGT/HLA | YGVNLYQSYGPSGQYTHEFDGDEQFYVDLGRKE | 806) |
| Accession No. | TVWCLPVLRQFRFDPQFALTNIAVLKHNLNSLIK | |
| HLA00613) | RSNSTAATNEVPEVTVFSKSPVTLGQPNILICLVD | |
| * Predicted signal | NIFPPVVNITWLSNGHSVTEGVSETSFLSKSDHSF | |
| peptide underlined | FKISYLTLLPSAEESYDCKVEHWGLDKPLLKHWE | |
| | PEIPAPMSELTETVVCALGLSVGLVGIVVGTVFII | |
| | RGLRSVGASRHQGPL | |
| DQA1*03:01 | MILNKALMLGALALTTVMSPCGGEDIVADHVAS | (SEQ ID NO: |
| (IMGT/HLA | YGVNLYQSYGPSGQYSHEFDGDEEFYVDLERKE | 807) |
| Accession No. | TVWQLPLFRRFRRFDPQFALTNIAVLKHNLNIVIK | |
| HLA00608) | RSNSTAATNEVPEVTVFSKSPVTLGQPNTLICLVD | |
| * Predicted signal | NIFPPVVNITWLSNGHSVTEGVSETSFLSKSDHSF | |
| peptide underlined | FKISYLTFLPSADEIYDCKVEHWGLDEPLLKHWE | |
| | PEIPTPMSELTETVVCALGLSVGLVGIVVGTVLIIR | |
| | GLRSVGASRHQGPL | |
| DQA1*01:02 | MILNKALLLGALALTTVMSPCGGEDIVADHVAS | (SEQ ID NO: |
| (IMGT/HLA | CGVNLYQFYGPSGQYTHEFDGDEQFYVDLERKE | 808) |
| Accession No. | TAWRWPEFSKFGGFDPQGALRNMAVAKHNLNI | |
| HLA00602) | MIKRYNSTAATNEVPEVTVFSKSPVTLGQPNTLI | |
| * Predicted signal | CLVDNIFPPVVNITWLSNGQSVTEGVSETSFLSKS | |
| peptide underlined | DHSFFKISYLTFLPSADEIYDCKVEHWGLDQPLL | |
| | KHWEPEIPAPMSELTETVVCALGLSVGLMGIVVG | |
| | TVFIIQGLRSVGASRHQGPL | |
| DQA1*02:01 | MILNKALMLGALALTTVMSPCGGEDIVADHVAS | (SEQ ID NO: |
| (IMGT/HLA | YGVNLYQSYGPSGQFTHEFDGDEEFYVDLERKE | 809) |
| Accession No. | TVWKLPLFHRLRFDPQFALTNIAVLKHNLNILIKR | |
| HLA00607) | SNSTAATNEVPEVTVFSKSPVTLGQPNTLICLVD | |
| * Predicted signal | NIFPPVVNITWLSNGHSVTEGVSETSFLSKSDHSF | |
| peptide underlined | FKISYLTFLPSADEIYDCKVEHWGLDEPLLKHWE | |
| | PEIPAPMSELTETVVCALGLSVGLVGIVVGTVLII | |
| | RGLRSVGASRHQGPL | |
| DQA1*01:01 | MILNKALLLGALALTTVMSPCGGEDIVADHVAS | (SEQ ID NO: |
| (IMGT/HLA | CGVNLYQFYGPSGQYTHEFDGDEEFYVDLERKE | 810) |
| Accession No. | TAWRWPEFSKFGGFDPQGALRNMAVAKHNLNI | |
| HLA00601) | MIKRYNSTAATNEVPEVTVFSKSPVTLGQPNTLI | |
| * Predicted signal | CLVDNIFPPVVNITWLSNGQSVTEGVSETSFLSKS | |
| peptide underlined | DHSFFKISYLTFLPSADEIYDCKVEHWGLDQPLL | |
| | KHWEPEIPAPMSELTETVVCALGLSVGLVGIVVG | |
| | TVFIIQGLRSVGASRHQGPL | |
| DQA1*01:03 | MILNKALLLGALALTTVMSPCGGEDIVADHVAS | (SEQ ID NO: |
| (IMGT/HLA | CGVNLYQFYGPSGQFTHEFDGDEQFYVDLEKKE | 811) |
| Accession No. | TAWRWPEFSKFGGFDPQGALRNMAVAKHNLNI | |
| HLA00604) | MIKRYNSTAATNEVPEVTVFSKSPVTLGQPNTLI | |
| * Predicted signal | CLVDNIFPPVVNITWLSNGHAVTEGVSETSFLSKS | |
| peptide underlined | DHSFFKISYLTFLPSADEIYDCKVEHWGLDQPLL | |
| | KHWEPEIPAPMSELTETVVCALGLSVGLVGIVVG | |
| | TVFIIQGLRSVGASRHQGPL | |
| DQA1*04:01 | MILNKALLLGALALTTVMSPCGGEDIVADHVAS | (SEQ ID NO: |
| (IMGT/HLA | YGVNLYQSYGPSGQYTHEFDGDEQFYVDLGRKE | 812) |
| Accession No. | TVWCLPVLRQFRFDPQFALTNIAVTKHNLNILIK | |
| HLA00612) | RSNSTAATNEVPEVTVFSKSPVTLGQPNTLICLVD | |
| * Predicted signal | NIFPPVVNITWLSNGHSVTEGVSETSFLSKSDHSF | |
| peptide underlined | FKISYLTFLPSADEIYDCKVEHWGLDEPLLKHWE | |
| | PEIPAPMSELTETVVCALGLSVGLVGIVVGTVFII | |
| | RGLRSVGASRHQGPL | |
| DQB1*03:01 | MSWKKALRIPGGLRAATVTLMLAMLSTPVAEGR | (SEQ ID NO: |
| (IMGT/HLA | DSPEDFVYQFKAMCYFTNGTERVRYVTRYIYNR | 813) |
| Accession No. | EEYARFDSDVEVYRAVTPLGPPDAEYWNSQKEV | |
| HLA00625) | LERTRAELDTVCRHNYQLELRTTLQRRVEPTVTI | |
| * Predicted signal | SPSRTEALNHHNLLVCSVTDFYPAQIKVRWFRND | |
| peptide underlined | QEETTGVVSTPLIRNGDWTFQILVMLEMTPQHG | |
| | DVYTCHVEHPSLQNPITVEWRAQSESAQSKMLS | |
| | GIGGFVLGLIFLGLGLIIHHRSQKGLLH | |
| DQB1*02:01 | MSWKKALRIPGGLRAATVTLMLSMLSTPVAEGR | (SEQ ID NO: |
| (IMGT/HLA | DSPEDFVYQFKGMCYFTNGTERVRLVSRSIYNRE | 814) |
| Accession No. | EIVRFDSDVGEFRAVTLLGLPAAEYWNSQKDILE | |
| HLA00622) | RKRAAVDRVCRHNYQLELRTTLQRRVEPTVTISP | |
| * Predicted signal | SRTEALNHHNLLVCSVTDFYPAQIKVRWFRNDQ | |
| peptide underlined | EETAGVVSTPLIRNGDWTFQILVMLEMTPQRGD | |
| | VYTCHVEHPSLQSPITVEWRAQSESAQSKMLSGI | |
| | GGFVLGLIFLGLGLIIHHRSQKGLLH | |
| DQB1*06:02 | MSWKKALRIPGDLRVATVTLMLAMLSSLLAEGR | (SEQ ID NO: |
| (IMGT/HLA | DSPEDFVFQFKGMCYFTNGTERVRLVTRYIYNRE | 815) |
| Accession No. | EYARFDSDVGVYRAVTPQGRPDAEYWNSQKEV | |
| HLA00646) | LEGTRAELDTVCRHNYEVAFRGILQRRVEPTVTI | |
| * Predicted signal | SPSRTEALNHHNLLVCSVTDFYPGQIKVRWFRND | |
| peptide underlined | QEETAGVVSTPLIRNGDWTFQILVMLEMTPQRG | |
| | DVYTCHVEHPSLQSPITVEWRAQSESAQSKMLSG | |
| | VGGFVLGLIFLGLGLIIRQRSQKGLLH | |
| DQB1*05:01 | MSWKKSLRIPGDLRVATVTLMLAILSSSLAEGRD | (SEQ ID NO: |
| (IMGT/HLA | SPEDFVYQFKGLCYFTNGTERVRGVTRHIYNREE | 816) |
| Accession No. | YVRFDSDVGVYRAVTPQGRPVAEYWNSQKEVL | |
| HLA00638) | EGARASVDRVCRHNYEVAYRGILQRRVEPTVTIS | |
| * Predicted signal | PSRTEALNHHNLLICSVTDFYPSQIKVRWFRNDQ | |
| peptide underlined | EETAGVVSTPLIRNGDWTFQILVMLEMTPQRGD | |
| | VYTCHVEHPSLQSPITVEWRAQSESAQSKMLSGV | |
| | GGFVLGLIFLGLGLIIRQRSRKGLLH | |
| DQB1*02:02 | MSWKKALRIPGGLRAATVTLMLSMLSTPVAEGR | (SEQ ID NO: |
| (IMGT/HLA | DSPEDFVYQFKGMCYFTNGTERVRLVSRSIYNRE | 817) |
| Accession No. | EIVRFDSDVGEFRAVTLLGLPAAEYWNSQKDILE | |
| HLA00623) | RKRAAVDRVCRHNYQLELRTTLQRRVEPTVTISP | |
| * Predicted signal | SRTEALNHHNLLVCSVTDFYPAQIKVRWFRNGQ | |
| peptide underlined | EETAGVVSTPLIRNGDWTFQILVMLEMTPQRGD | |
| | VYTCHVEHPSLQSPITVEWRAQSESAQSKMLSGI | |
| | GGFVLGLIFLGLGLIIHHRSQKGLLH | |
| DQB1*03:02 | MSWKKALRIPGGLRVATVTLMLAMLSTPVAEGR | (SEQ ID NO: |
| (IMGT/HLA | DSPEDFVYQFKGMCYFTNGTERVRLVTRYIYNR | 818) |
| Accession No. | EEYARFDSDVGVYRAVTPLGPPAAEYWNSQKEV | |
| HLA00627) | LERTRAELDTVCRHNYQLELRTTLQRRVEPTVTI | |
| * Predicted signal | SPSRTEALNHHNLLVCSVTDFYPAQIKVRWFRND | |
| peptide underlined | QEETTGVVSTPLIRNGDWTFQILVMLEMTPQRGD | |
| | VYTCHVEHPSLQNPIIVEWRAQSESAQSKMLSGI | |
| | GGFVLGLIFLGLGLIIHHRSQKGLLH | |
| DQB1*06:03 | MSWKKALRIPGDLRVATVTLMLAMLSSLLAEGR | (SEQ ID NO: |
| (IMGT/HLA | DSPEDFVYQFKGMCYFTNGTERVRLVTRHIYNR | 819) |
| Accession No. | EEYARFDSDVGVYRAVTPQGRPDAEYWNSQKE | |
| HLA00647) | VLEGTRAELDTVCRHNYEVAFRGILQRRVEPTVT | |
| * Predicted signal | ISPSRTEALNHHNLLVCSVTDFYPGQIKVRWFRN | |
| peptide underlined | DQEETAGVVSTPLIRNGDWTFQILVMLEMTPQR | |
| | GDVYTCHVEHPSLQSPITVEWRAQSESAQSKMLS | |
| | GVGGFVLGLIFLGLGLIIRQRSQKGLLH | |
| DQB1*03:03 | MSWKKALRIPGGLRVATVTLMLAMLSTPVAEGR | (SEQ ID NO: |
| (IMGT/HLA | DSPEDFVYQFKGMCYFTNGTERVRLVTRYIYNR | 820) |
| Accession No. | EEYARFDSDVGVYRAVTPLGPPDAEYWNSQKEV | |
| HLA00629) | LERTRAELDTVCRHNYQLELRTTLQRRVEPTVTI | |
| * Predicted signal | SPSRTEALNHHNLLVCSVTDFYPAQIKVRWFRND | |
| peptide underlined | QEETTGVVSTPLIRNGDWTFQILVMLEMTPQRGD | |
| | VYTCHVEHPSLQNPIIVEWRAQSESAQSKMLSGI | |
| | GGFVLGLIFLGLGLIIHHRSQKGLLH | |
| DQB1*06:04 | MSWKKALRIPGDLRVATVTLMLAMLSSLLAEGR | (SEQ ID NO: |
| (IMGT/HLA | DSPEDFVYQFKGMCYFTNGTERVRLVTRHIYNR | 821) |
| Accession No. | EEYARFDSDVGVYRAVTPQGRPVAEYWNSQKE | |
| HLA00648) | VLERTRAELDTVCRHNYEVGYRGILQRRVEPTV | |
| * Predicted signal | TISPSRTEALNHHNLLVCSVTDFYPGQIKVQWFR | |
| peptide underlined | NDQEETAGVVSTPLIRNGDWTFQILVMLEMTPQ | |
| | RGDVYTCHVEHPSLQSPITVEWRAQSESAQSKM | |
| | LSGVGGFVLGLIFLGLGLIIRQRSQKGLLH | |
| DQB1*05:03 | MSWKKSLRIPGDLRVATVTLMLAILSSSLAEGRD | (SEQ ID NO: |
| (IMGT/HLA | SPEDFVYQFKGLCYFTNGTERVRGVTRHIYNREE | 822) |
| Accession No. | YVRFDSDVGVYRAVTPQGRPDAEYWNSQKEVL | |
| HLA00640) | EGARASVDRVCRHNYEVAYRGILQRRVEPTVTIS | |
| * Predicted signal | PSRTEALNHHNLLICSVTDFYPSQIKVRWFRNDQ | |
| peptide underlined | EETAGVVSTPLIRNGDWTFQILVMLEMTPQRGD | |
| | VYTCHVEHPSLQSPITVEWRAQSESAQSKMLSGV | |
| | GGFVLGLIFLGLGLIIRQRSRKGPQGPPPAGLLH | |
| DQB1*04:02 | MSWKKALRIPGGLRVATVTLMLAMLSTPVAEGR | (SEQ ID NO: |
| (IMGT/HLA | DSPEDFVFQFKGMCYFTNGTERVRGVTRYIYNR | 823) |
| Accession No. | EEYARFDSDVGVYRAVTPLGRLDAEYWNSQKDI | |
| HLA00637) | LEEDRASVDTVCRHNYQLELRTTLQRRVEPTVTI | |
| * Predicted signal | SPSRTEALNHHNLLVCSVTDFYPAQIKVRWFRND | |
| peptide underlined | QEETTGVVSTPLIRNGDWTFQILVMLEMTPQRGD | |
| | VYTCHVEHPSLQNPIIVEWRAQSESAQSKMLSGI | |
| | GGFVLGLIFLGLGLIIHHRSQKGLLH | |
| DPA1*01:03 | MRPEDRMFHIRAVILRALSLAFLLSLRGAGAIKA | (SEQ ID NO: |
| (IMGT/HLA | DHVSTYAAFVQTHRPTGEFMFEFDEDEMFYVDL | 824) |
| Accession No. | DKKETVWHLEEFGQAFSFEAQGGLANIAILNNNL | |
| HLA00499) | NTLIQRSNHTQATNDPPEVTVFPKEPVELGQPNT | |
| * Predicted signal | LICHIDKFFPPVLNVTWLCNGELVTEGVAESLFLP | |
| peptide underlined | RTDYSFHKFHYLTFVPSAEDFYDCRVEHWGLDQ | |
| | PLLKHWEAQEPIQMPETTETVLCALGLVLGLVGI | |
| | IVGTVLIIKSLRSGHDPRAQGTL | |
| DPA1*02:01 | MRPEDRMFHIRAVILRALSLAFLLSLRGAGAIKA | (SEQ ID NO: |
| (IMGT/HLA | DHVSTYAAFVQTHRPTGEFMFEFDEDEQFYVDL | 825) |
| Accession No. | DKKETVWHLEEFGRAFSFEAQGGLANIAILNNNL | |
| HLA00504) | NTLIQRSNHTQAANDPPEVTVFPKEPVELGQPNT | |
| * Predicted signal | LICHIDRFFPPVLNVTWLCNGEPVTEGVAESLFLP | |
| peptide underlined | RTDYSFHKFHYLTFVPSAEDVYDCRVEHWGLDQ | |
| | PLLKHWEAQEPIQMPETTETVLCALGLVLGLVGI | |
| | IVGTVLIIKSLRSGHDPRAQGPL | |
| DPA1*02:07 | MRPEDRMFHIRAVILRALSLAFLLSLRGAGAIKA | (SEQ ID NO: |
| (IMGT/HLA | DHVSTYAMFVQTHRPTGEFMFEFDEDEQFYVDL | 826) |
| Accession No. | DKKETVWHLEEFGRAFSFEAQGGLANIAILNNNL | |
| HLA15619) | NTLIQRSNHTQAANDPPEVTMFPKEPVELGQPNT | |
| * Predicted signal | LICHIDRFFPPVLNVTWLCNGEPVTEGVAESLFLP | |
| peptide underlined | RTDYSFHKFHYLTFVPSAEDVYDCRVEHWGLDQ | |
| | PLLKHWEAQEPIQMPETTETVLCALGLVLGLVGI | |
| | IVGTVLIIKSLRSGHDPRAQGPL | |
| DPB1*04:01 | MMVLQVSAAPRTVALTALLMVLLTSVVQGRAT | (SEQ ID NO: |
| (IMGT/HLA | PENYLFQGRQECYAFNGTQRFLERYIYNREEFAR | 827) |
| Accession No. | FDSDVGEFRAVTELGRPAAEYWNSQKDILEEKR | |
| HLA00521) | AVPDRMCRHNYELGGPMTLQRRVQPRVNVSPS | |
| * Predicted signal | KKGPLQHHNLLVCHVTDFYPGSIQVRWFLNGQE | |
| peptide underlined | ETAGVVSTNLIRNGDWTFQILVMLEMTPQQGDV | |
| | YTCQVEHTSLDSPVTVEWKAQSDSARSKTLTGA | |
| | GGFVLGLIICGVGIFMHRRSKKVQRGSA | |
| DPB1*02:01 | MMVLQVSAAPRTVALTALLMVLLTSVVQGRAT | (SEQ ID NO: |
| (IMGT/HLA | PENYLFQGRQECYAFNGTQRFLERYIYNREEFVR | 828) |
| Accession No. | FDSDVGEFRAVTELGRPDEEYWNSQKDILEEERA | |
| HLA00517) | VPDRMCRHNYELGGPMTLQRRVQPRVNVSPSK | |
| * Predicted signal | KGPLQHHNLLVCHVTDFYPGSIQVRWFLNGQEE | |
| peptide underlined | TAGVVSTNLIRNGDWTFQILVMLEMTPQQGDVY | |
| | TCQVEHTSLDSPVTVEWKAQSDSARSKTLTGAG | |
| | GFVLGLIICGVGIFMHRRSKKVQRGSA | |
| DPB1*04:02 | MMVLQVSAAPRTVALTALLMVLLTSVVQGRAT | (SEQ ID NO: |
| (IMGT/HLA | PENYLFQGRQECYAFNGTQRFLERYIYNREEFVR | 829) |
| Accession No. | FDSDVGEFRAVTELGRPDEEYWNSQKDILEEKR | |
| HLA00522) | AVPDRMCRHNYELGGPMTLQRRVQPRVNVSPS | |
| * Predicted signal | KKGPLQHHNLLVCHVTDFYPGSIQVRWFLNGQE | |
| peptide underlined | ETAGVVSTNLIRNGDWTFQILVMLEMTPQQGDV | |
| | YTCQVEHTSMDSPVTVEWKAQSDSARSKTLTGA | |
| | GGFVLGLIICGVGIFMHRRSKKVQRGSA | |
| DPB1*03:01 | MMVLQVSAAPRTVALTALLMVLLTSVVQGRAT | (SEQ ID NO: |
| (IMGT/HLA | PENYVYQLRQECYAFNGTQRFLERYIYNREEFVR | 830) |
| Accession No. | FDSDVGEFRAVTELGRPDEDYWNSQKDLLEEKR | |
| HLA00520) | AVPDRVCRHNYELDEAVTLQRRVQPKVNVSPSK | |
| * Predicted signal | KGPLQHHNLLVCHVTDFYPGSIQVRWFLNGQEE | |
| peptide underlined | TAGVVSTNLIRNGDWTFQILVMLEMTPQQGDVY | |
| | ICQVEHTSLDSPVTVEWKAQSDSARSKTLTGAG | |
| | GFVLGLIICGVGIFMHRRSKKVQRGSA | |
| DPB1*01:01 | MMVLQVSAAPRTVALTALLMVLLTSVVQGRAT | (SEQ ID NO: |
| (IMGT/HLA | PENYVYQGRQECYAFNGTQRFLERYIYNREEYA | 831) |
| Accession No. | RFDSDVGEFRAVTELGRPAAEYWNSQKDILEEK | |
| HLA00514) | RAVPDRVCRHNYELDEAVTLQRRVQPKVNVSPS | |
| * Predicted signal | KKGPLQHHNLLVCHVTDFYPGSIQVRWFLNGQE | |
| peptide underlined | ETAGVVSTNLIRNGDWTFQILVMLEMTPQQGDV | |
| | YICQVEHTSLDSPVTVEWKAQSDSAQSKTLTGA | |
| | GGFVLGLIICGVGIFMHRRSKKVQRGSA | |
| DPB1*11:01 | MMVLQVSAAPRTVALTALLMVLLTSVVQGRAT | (SEQ ID NO: |
| (IMGT/HLA | PENYVYQLRQECYAFNGTQRFLERYIYNRQEYA | 832) |
| Accession No. | RFDSDVGEFRAVTELGRPAAEYWNSQKDLLEER | |
| HLA00528) | RAVPDRMCRHNYELDEAVTLQRRVQPKVNVSPS | |
| * Predicted signal | KKGPLQHHNLLVCHVTDFYPGSIQVRWFLNGQE | |
| peptide underlined | ETAGVVSTNLIRNGDWTFQILVMLEMTPQQGDV | |
| | YICQVEHTSLDSPVTVEWKAQSDSARSKTLTGA | |
| | GGFVLGLIICGVGIFMHRRSKKVQRGSA | |
| DPB1*05:01 | MMVLQVSAAPRTVALTALLMVLLTSVVQGRAT | (SEQ ID NO: |
| (IMGT/HLA | PENYLFQGRQECYAFNGTQRFLERYIYNREELVR | 833) |
| Accession No. | FDSDVGEFRAVTELGRPEAEYWNSQKDILEEKR | |
| HLA00523) | AVPDRMCRHNYELDEAVTLQRRVQPKVNVSPSK | |
| * Predicted signal | KGPLQHHNLLVCHVTDFYPGSIQVRWFLNGQEE | |
| peptide underlined | TAGVVSTNLIRNGDWTFQILVMLEMTPQQGDVY | |
| | ICQVEHTSLDSPVTVEWKAQSDSARSKTLTGAG | |
| | GFMLGLIICGVGIFMHRRSKKVQRGSA | |
| DPB1*10:01 | MMVLQVSAAPRTVALTALLMVLLTSVVQGRAT | (SEQ ID NO: |
| (IMGT/HLA | PENYVHQLRQECYAFNGTQRFLERYIYNREEFVR | 834) |
| Accession No. | FDSDVGEFRAVTELGRPDEEYWNSQKDILEEERA | |
| HLA00527) | VPDRVCRHNYELDEAVTLQRRVQPKVNVSPSKK | |
| * Predicted signal | GPLQHHNLLVCHVTDFYPGSIQVRWFLNGQEET | |
| peptide underlined | AGVVSTNLIRNGDWTFQILVMLEMTPQQGDVYI | |
| | CQVEHTSLDSPVTVEWKAQSDSARSKTLTGAGG | |
| | FVLGLIICGVGIFMHRRSKKVQRGSA | |
| DPB1*06:01 | MMVLQVSAAPRTVALTALLMVLLTSVVQGRAT | (SEQ ID NO: |
| (IMGT/HLA | PENYVYQLRQECYAFNGTQRFLERYIYNREEFVR | 835) |
| Accession No. | FDSDVGEFRAVTELGRPDEDYWNSQKDLLEEER | |
| HLA00524) | AVPDRMCRHNYELDEAVTLQRRVQPKVNVSPSK | |
| * Predicted signal | KGPLQHHNLLVCHVTDFYPGSIQVRWFLNGQEE | |
| peptide underlined | TAGVVSTNLIRNGDWTFQILVMLEMTPQQGDVY | |
| | ICQVEHTSLDSPVTVEWKAQSDSARSKTLTGAG | |
| | GFVLGLIICGVGIFMHRRSKKVQRGSA | |
| DPB1*13:01 | MMVLQVSAAPRTVALTALLMVLLTSVVQGRAT | (SEQ ID NO: |
| (IMGT/HLA | PENYVYQLRQECYAFNGTQRFLERYIYNREEYA | 836) |
| Accession No. | RFDSDVGEFRAVTELGRPAAEYWNSQKDILEEER | |
| HLA00530) | AVPDRICRHNYELDEAVTLQRRVQPKVNVSPSK | |
| * Predicted signal | KGPLQHHNLLVCHVTDFYPGSIQVRWFLNGQEE | |
| peptide underlined | TAGVVSTNLIRNGDWTFQILVMLEMTPQQGDVY | |
| | ICQVEHTSLDSPVTVEWKAQSDSARSKTLTGAG | |
| | GFVLGLIICGVGIFMHRRSKKVQRGSA | |
| DPB1*14:01 | MMVLQVSAAPRTVALTALLMVLLTSVVQGRAT | (SEQ ID NO: |
| (IMGT/HLA | PENYVHQLRQECYAFNGTQRFLERYIYNREEFVR | 837) |
| Accession No. | FDSDVGEFRAVTELGRPDEDYWNSQKDLLEEKR | |
| HLA00531) | AVPDRVCRHNYELDEAVTLQRRVQPKVNVSPSK | |
| * Predicted signal | KGPLQHHNLLVCHVTDFYPGSIQVRWFLNGQEE | |
| peptide underlined | TAGVVSTNLIRNGDWTFQILVMLEMTPQQGDVY | |
| | ICQVEHTSLDSPVTVEWKAQSDSARSKTLTGAG | |
| | GFVLGLIICGVGIFMHRRSKKVQRGSA | |
| DPB1*17:01 | MMVLQVSAAPRTVALTALLMVLLTSVVQGRAT | (SEQ ID NO: |
| (IMGT/HLA | PENYVHQLRQECYAFNGTQRFLERYIYNREEFVR | 838) |
| Accession No. | FDSDVGEFRAVTELGRPDEDYWNSQKDILEEER | |
| HLA00534) | AVPDRMCRHNYELDEAVTLQRRVQPRVNVSPSK | |
| * Predicted signal | KGPLQHHNLLVCHVTDFYPGSIQVRWFLNGQEE | |
| peptide underlined | TAGVVSTNLIRNGDWTFQILVMLEMTPQQGDVY | |
| | TCQVEHTSLDSPVTVEWKAQSDSARSKTLTGAG | |
| | GFVLGLIICGVGIFMHRRSKKVQRGSA | |

Additional MHC allele amino acid sequences are known in the art and are available, for example at the IMGT/HLA Database (available on the world wide web at ebi.ac.uk/ipd/imgt/hla/; see Robinson et al. Nucl. Acids Res. 43: D423-431 (2015)).

In certain embodiments, the polypeptide is an exogenous antigen-presenting polypeptide as described herein. An exemplary exogenous antigen-presenting polypeptide includes:

a) a naturally occurring form of the human polypeptide;

b) the human polypeptide having a sequence appearing in a database, e.g., GenBank database, on Dec. 22, 2017;

c) a human polypeptide having a sequence that differs by no more than 1, 2, 3, 4, 5 or 10 amino acid residues from a sequence of a) or b);

d) a human polypeptide having a sequence that differs at no more than 1, 2, 3, 4, 5 or 10% its amino acids residues from a sequence of a) or b);

e) a human polypeptide having a sequence that does not differ substantially from a sequence of a) or b); or

f) a human polypeptide having a sequence of c), d), or e) that does not differ substantially in a biological activity, e.g., an enzymatic activity (e.g., specificity or turnover) or binding activity (e.g., binding specificity or affinity for an antigenic peptide) from a human polypeptide having the sequence of a) or b). Candidate peptides under f) can be made and screened for similar activity as described herein and would be equivalent hereunder if expressed in engineered erythroid cells as described herein).

In embodiments, an exogenous antigen-presenting polypeptide comprises a human polypeptide or fragment thereof, e.g., all or a fragment of a human polypeptide of a), b), c), d), e), or f) of the preceding paragraph. In an embodiment, the exogenous antigen-presenting polypeptide comprises a fusion polypeptide comprising all or a fragment of a human polypeptide of a), b), c), d), e), or f) of the preceding paragraph and additional amino acid sequence. In an embodiment the additional amino acid sequence comprises all or a fragment of human polypeptide of a), b), c), d), e), or f) of the preceding paragraph for a different human exogenous antigen-presenting polypeptide.

In some embodiments of the present disclosure, the artificial antigen presenting cell comprises an erythroid cell or enucleated cell that does not comprise an endogenous antigen presenting polypeptide (e.g. a MHC class I or MHC class II molecule). In some embodiments, the artificial antigen presenting cell comprises an erythroid cell or enucleated cell that has been derived from an erythroid precursor cell that has not been genetically modified to delete and/or alter expression of an endogenous antigen presenting polypeptide (e.g. a MHC class I or MHC class II molecule).

### Exogenous Costimulatory Polypeptides

An exogenous costimulatory polypeptide includes a polypeptide on an antigen presenting cell (e.g., an aAPC) that specifically binds a cognate costimulatory molecule on a T cell (e.g., an MHC molecule, B and T lymphocyte attenuator (CD272) and a Toll ligand receptor), thereby providing a signal which mediates a T cell response, including, but not limited to, proliferation, activation, differentiation, and the like. A costimulatory polypeptide also encompasses, inter alia, an antibody that specifically binds with a costimulatory molecule present on a T cell. Such antibody preferably binds and acts as an agonist to the costimulatory molecule on the T cell.

In some embodiments, the desired response is cell death, e.g., of an infected cell. In some embodiments, the costimulatory polypeptides trigger multiple T cell activation pathways to induce an immune response. In some embodiments, the aAPC comprising, inter alia, costimulatory polypeptides, promotes T cell proliferation. In embodiments, one or more (e.g., 2, 3, 4, or 5 or more) costimulatory polypeptides comprise an activating polypeptide of Table 6, below, or a T-cell activating variant (e.g., fragment) thereof. In embodiments, one or more (e.g., 2, 3, 4, or 5 or more) costimulatory polypeptides comprise an antibody molecule (e.g. agonizing antibody) that binds a target receptor of Table 6 or a T-cell activating variant (e.g., fragment) thereof. In some embodiments, the costimulatory polypeptides comprise different T cell activation ligands, e.g. one or more activating polypeptides of Table 6, in any combination thereof, to stimulate T cells. In some embodiments, the aAPC comprises an erythroid cell (e.g. an enucleated cell) that presents, e.g. comprises on the cell surface, 4-1BBL, OX40L, and CD40L, or fragments or variants thereof. In embodiments, these proteins signal through complementary activation pathways. The costimulatory polypeptides can be derived from endogenous T cell activation ligands or from antibody molecules to the target receptors.

TABLE 6
Costimulatory Polypeptides

| Activating Polypeptide (Ligand) | Target Receptor on T cell |
|---|---|
| B7-H2 (e.g., Accession Number | ICOS, CD28 (e.g., Accession Number |
| NP_056074.1) | NP_006130.1) |
| B7-1 (e.g., Accession Number NP_005182.1) | CD28 (e.g., Accession Number |
| | NP_006130.1) |
| B7-2 (e.g., Accession Number AAA86473) | CD28 (e.g., Accession Number |
| | NP_006130.1) |
| CD70 (e.g., Accession Number | CD27 (e.g., Accession Number |
| NP_001243.1) | NP_001233.1) |
| LIGHT (e.g., Accession Number | HVEM (e.g., Accession Number |
| NP_003798.2) | AAQ89238.1) |
| HVEM (e.g., Accession Number | LIGHT (e.g., Accession Number |
| AAQ89238.1) | NP_003798.2) |
| CD40L (e.g., Accession Number | CD40 (e.g., Accession Number |
| BAA06599.1) | NP_001241.1) |
| 4-1BBL (e.g., Accession Number | 4-1BB (e.g., Accession NP_001552.2) |
| NP_003802.1) | |
| OX40L (e.g., Accession Number | OX40 (e.g., Accession Number |
| NP_003317.1) | NP_003318.1) |
| TL1A (e.g., Accession Number | DR3 (e.g., Accession Number NP_683866.1) |
| NP_005109.2) | |
| GITRL (e.g., Accession Number | GITR (e.g., Accession Number |
| NP_005083.2) | NP_004186.1) |
| CD30L (e.g., Accession Number | CD30 (e.g., Accession Number |
| NP_001235.1), | NP_001234.3) |
| TIM4 (e.g., Accession Number | TIM1 (e.g., Accession Number |
| NP_612388.2) | NP_036338.2) |
| SLAM (e.g., Accession Number | SLAM (e.g., Accession Number |
| AAK77968.1) | AAK77968.1) |
| CD48 (e.g., Accession Number | CD2 (e.g., Accession Number |
| CAG33293.1) | NP_001315538.1) |
| CD58 (e.g., Accession Number | CD2 (e.g., Accession Number |
| CAG33220.1) | NP_001315538.1) |
| CD155 (e.g., Accession Number | CD226 (e.g., Accession Number |
| NP_001129240.1) | NP_006557.2) |
| CD112 (e.g., Accession Number | CD226 (e.g., Accession Number |
| NP_001036189.1) | NP_006557.2) |
| CD137L (e.g., Accession Number | CD137 (e.g., Accession NP_001552.2) |
| NP_003802.1) | |

In some embodiments, the polypeptide comprising 4-1BBL is an N-terminal truncated 4-1BBL (e.g. SEQ ID NO: 851). In some embodiments, the polypeptide comprising 4-1BBL is full length 4-1BBL.

In some embodiments, the one or more costimulatory polypeptides comprises an activating cytokine, interferon or TNF family member, e.g., IFNα, IL2, IL6 or any combination thereof. Activating cytokines, interferons and TNF family members which are useful in the invention are discussed further below. In embodiments, the one or more costimulatory polypeptides comprises one or more activating cytokine, interferon or TNF family member, and further comprises one or more activating polypeptide or ligand (e.g., of Table 6) or a T-cell activating variant (e.g., fragment) thereof, or one or more antibody molecules (e.g. agonizing antibody) that binds a target costimulatory T cell receptor (e.g., of Table 6) or a T-cell activating variant (e.g., fragment) thereof.

T-Cell Expansion

In certain embodiments, the disclosure features aAPCs that can be used to specifically induce proliferation of a T cell expressing a known co-stimulatory molecule. The method comprises contacting a T cell that is to be expanded with an aAPC presenting (e.g. comprising on the cell surface) an exogenous polypeptide that specifically binds with the co-stimulatory molecule expressed by the T-cell. Thus, contacting a T cell with an aAPC comprising, among other things, a costimulatory ligand that specifically binds a cognate costimulatory molecule expressed on the T cell surface, stimulates the T cell and induces T cell proliferation such that large numbers of specific T cells can be readily produced. The aAPC expands the T cell “specifically” in that only the T cells expressing the particular costimulatory molecule are expanded by the aAPC. Thus, where the T cell to be expanded is present in a mixture of cells, some or most of which do not express the costimulatory molecule, only the T cell of interest will be induced to proliferate and expand in cell number. The T cell can be further purified using a wide variety of cell separation and purification techniques, such as those known in the art and/or described elsewhere herein.

As would be appreciated by the skilled artisan, based upon the disclosure provided herein, the T cell of interest need not be identified or isolated prior to expansion using the aAPC. This is because the aAPC is selective and will only expand the T cell(s) expressing the cognate costimulatory molecule.

In certain embodiments, the polypeptide is an exogenous costimulatory polypeptide as described herein. An exemplary costimulatory polypeptide includes:

a) a naturally occurring form of the human polypeptide;

b) the human polypeptide having a sequence appearing in a database, e.g., GenBank database, on Dec. 22, 2017;

c) a human polypeptide having a sequence that differs by no more than 1, 2, 3, 4, 5 or 10 amino acid residues from a sequence of a) or b);

d) a human polypeptide having a sequence that differs at no more than 1, 2, 3, 4, 5 or 10% its amino acids residues from a sequence of a) or b);

e) a human polypeptide having a sequence that does not differ substantially from a sequence of a) or b); or

f) a human polypeptide having a sequence of c), d), or e) that does not differ substantially in a biological activity, e.g., an enzymatic activity (e.g., specificity or turnover) or binding activity (e.g., binding specificity or affinity) from a human polypeptide having the sequence of a) or b). Candidate peptides under f) can be made and screened for similar activity as described herein and would be equivalent hereunder if expressed in engineered erythroid cells as described herein).

In embodiments, an exogenous costimulatory polypeptide comprises a human polypeptide or fragment thereof, e.g., all or a fragment of a human polypeptide of a), b), c), d), e), or f) of the preceding paragraph. In an embodiment, the exogenous costimulatory polypeptide comprises a fusion polypeptide comprising all or a fragment of a human polypeptide of a), b), c), d), e), or f) of the preceding paragraph and additional amino acid sequence. In an embodiment the additional amino acid sequence comprises all or a fragment of human polypeptide of a), b), c), d), e), or f) of the preceding paragraph for a different human costimulatory polypeptide.

In embodiments, an aAPC cell targets multiple T cell activating pathways in combination (e.g., as described in Table 6, above), e.g., using ligands or antibody molecules, or both, co-expressed (or co-presented) on an aAPC.

In some embodiments, the at least one exogenous costimulatory polypeptide is selected from the group consisting of 4-1BBL, LIGHT, CD80, CD86, CD70, IL-7, IL-12, OX40L, GITRL, TIM4, SLAM, CD48, CD58, CD83, CD155, CD112, IL-15Rα fused to IL-15, IL-2, IL-21, a ligand for ICAM-1, a ligand for LFA-1, and combinations thereof. In some embodiments, the at least one exogenous costimulatory polypeptide is an agonist antibody to the cognate costimulatory ligand receptor. For example, in certain embodiments, the costimulatory polypeptide is an agonist antibody to 4-1-BB, LIGHT receptor (HVEM), CD80 receptor, CD86 receptor, OX40, GITR, TIM4 receptor (TIM1), SLAM receptor, CD48 receptor (CD2), CD58 receptor (CD2), CD 83 receptor, CD155 receptor (CD226), CD112 receptor (CD226), IL-2 receptor (CD25, CD122, CD132), IL-21 receptor, ICAM, and combinations thereof. In certain embodiments, the at least one exogenous costimulatory polypeptides is an anti CD3 antibody or an anti-CD38 antibody and combinations thereof. In another embodiment, the aAPC presents, e.g. comprises on the cell surface, at least two, at least 3, at least 4, or at least 5 exogenous costimulatory polypeptides. In some embodiments, the costimulatory proteins are fused to each other, for example IL-21 fused to IL-2.

In some embodiments, the one or more costimulatory polypeptides include or are fused to a membrane anchor. In some embodiments, the membrane anchor is selected from a sequence set forth in Table 3. In some embodiments, the one or more costimulatory polypeptides include or are fused to a leader sequence. In some embodiments, the leader sequence is selected from a sequence set forth in Table 2.

### Exogenous Co-Inhibitory Polypeptides

An exogenous co-inhibitory polypeptide is any polypeptide that suppresses a T cell, including inhibition of T cell activity, inhibition of T cell proliferation, anergizing of a T cell, or induction of apoptosis of a T cell.

In some embodiments, an exogenous co-inhibitory polypeptide is an inhibitory polypeptide ligand on an antigen presenting cell that specifically binds a cognate coinhibitory molecule on a T cell. In some embodiments, the co-inhibitory polypeptide ligand is an inhibitory polypeptide shown in Table 7.

In some embodiments, an exogenous co-inhibitory polypeptide is an agonist (e.g. an antibody) that specifically binds a coinhibitory receptor on a T cell. In some embodiments, the agonist is an antibody that binds a receptor selected from the group consisting of: PD1, CTLA4, TIM3, CEACAM (e.g., CEACAM-1, CEACAM-3 and/or CEACAM-5), LAGS, VISTA, BTLA, TIGIT, LAIR1, CD160, and 2B4. In other embodiments, the agonist is an antibody that binds a target receptor on a T cell shown in Table 7.

TABLE 7
Co-inhibitory Polypeptides

| Inhibitory Polypeptide | Target Receptor on T cell |
|---|---|
| B7-1 | CTLA4, B7H1 |
| B7-2 | CTLA4 |
| B7DC | PD1 |
| B7H1 | PD1, B7-1 |
| HVEM | CD160, BTLA |
| COLLAGEN | LAIR1 |
| GALECTIN9 | TIM3 |
| CD48, TIM4 | TIM4R |
| CD48 | 2B4 |
| CD155, CD112, CD113 | TIGIT |
| PDL1 | PD1 |
| | LAG3 |

In some embodiments, an exogenous co-inhibitory polypeptide is an antibody that blocks binding of a costimulatory polypeptide to its cognate costimulatory receptor. In various embodiments, the exogenous co-inhibitory polypeptide is an antibody that blocks binding of 4-1BBL, LIGHT, CD80, CD86, CD70, OX40L, GITRL, TIM4, SLAM, CD48, CD58, CD83, CD155, CD112, IL-15Rα fused to IL-15, IL-2, IL-21, ICAM, a ligand for LFA-1, an anti CD3 antibody or an anti CD28 antibody, to its receptor.

In other embodiments, the co-inhibitory polypeptide is selected from IL-35, IL-10, or VSIG-3.

In some embodiments, the exogenous co-inhibitory polypeptide is VSIG-3.

In other embodiments, an aAPC cell targets multiple T cell inhibitory pathways in combination (e.g., as described in Table 7, above), e.g., using ligands or antibody molecules, or both, co-expressed on an aAPC.

In certain embodiments, the polypeptide is an exogenous coinhibitory polypeptide as described herein. An exemplary coinhibitory polypeptide includes:

a) a naturally occurring form of the human polypeptide;

b) the human polypeptide having a sequence appearing in a database, e.g., GenBank database, on Dec. 22, 2017;

c) a human polypeptide having a sequence that differs by no more than 1, 2, 3, 4, 5 or 10 amino acid residues from a sequence of a) or b);

d) a human polypeptide having a sequence that differs at no more than 1, 2, 3, 4, 5 or 10% its amino acids residues from a sequence of a) or b);

e) a human polypeptide having a sequence that does not differ substantially from a sequence of a) or b); or

f) a human polypeptide having a sequence of c), d), or e) that does not differ substantially in a biological activity, e.g., an enzymatic activity (e.g., specificity or turnover) or binding activity (e.g., binding specificity or affinity) from a human polypeptide having the sequence of a) or b). Candidate peptides under f) can be made and screened for similar activity as described herein and would be equivalent hereunder if expressed in engineered erythroid cells as described herein).

In embodiments, an exogenous coinhibitory polypeptide comprises a human polypeptide or fragment thereof, e.g., all or a fragment of a human polypeptide of a), b), c), d), e), or f) of the preceding paragraph. In an embodiment, the exogenous coinhibitory polypeptide comprises a fusion polypeptide comprising all or a fragment of a human polypeptide of a), b), c), d), e), or f) of the preceding paragraph and additional amino acid sequence. In an embodiment the additional amino acid sequence comprises all or a fragment of human polypeptide of a), b), c), d), e), or f) of the preceding paragraph for a different human coinhibitory polypeptide.

In some embodiments, the aAPC presents, e.g. comprises on the cell surface, at least two, at least 3, at least 4, or at least 5 exogenous co-inhibitory polypeptides.

In some embodiments, the one or more co-inhibitory polypeptides include or are fused to a membrane anchor. In some embodiments, the membrane anchor is selected from a sequence set forth in Table 3. In some embodiments, the one or more co-inhibitory polypeptides include or are fused to a leader sequence. In some embodiments, the leader sequence is selected from a sequence set forth in Table 2.

### T-Cell Activation Signals

For efficient induction of T-cell proliferation, activation and expansion, several signals need to be transmitted from the aAPC to naïve T cells. These signals are commonly referred to as Signal 1, Signal 2 and Signal 3, and are described below. In some embodiments, the aAPCs described herein comprise one or more exogenous polypeptides comprising Signal 1, one or more exogenous polypeptides comprising Signal 2, and/or one or more exogenous polypeptides comprising Signal 3, in any combination as set forth below. In some embodiments, in addition to Signal 1, Signal 2 and Signal 3, the aAPCs described herein further comprise one or more exogenous polypeptides comprising one or more cell adhesion molecules to further facilitate the interation between T-cells and the aAPCs. It is to be understood that when an aAPC comprises the one or more exogenous polypeptides comprising Signal 1 and/or the one or more exogenous polypeptides comprising Signal 2 and/or the one or more exogenous polypeptides comprising Signal 3 (and optionally the one or more polypeptides comprising a cell adhesion molecules), the exogenous polypeptides comprising Signal 1 and/or Signal 2 and/or Signal 3 and/or a cell adhesion molecule are all comprised on the same aAPC.

The aAPCs described herein offer numerous advantages over the use of spherical nanoparticles, such as rigid, bead-based aAPCs. For example, the membrane of an aAPC as described herein (i.e., an engineered erythroid cell or enucleated cell) is much more dynamic and fluid than the outer surface of a nanoparticle, which is rigid and immobile, and therefore limits the movement of the polypeptides on its surface. The fluidity of the aAPC membrane allows for greater molecular mobility and more efficient molecular reorganization, and is advantageous for immunological synapse formation and T cell stimulation. In some embodiments, the aAPCs described herein comprising one or more exogenous polypeptides comprising Signal 1, one or more exogenous polypeptides comprising Signal 2, and/or one or more exogenous polypeptides comprising Signal 3, in any combination as set forth below, on the surface of the cells, provide a more controlled stimulation of T-cells, thereby allowing for the propagation of T-cells with a specific phenotype and activity. In some embodiments, by engineering the aAPCs to comprise Signal 1 and/or Signal 2 and/or Signal 3 on the surface of the cell, the aAPCs provide optimal control over the signals provided to T-cells.

Signal 1—Antigen Recognition

T cell activation occurs after a T cell receptor (TCR) recognizes a specific peptide antigen presented on MHC complexes of an aAPC as described herein. Generally, exogenous antigenic polypeptides presented on MHC class II are recognized by the TCR in conjunction with the CD4 T cell co-receptor. Exogenous antigenic polypeptides presented on MHC class I are recognized by the TCR in conjunction with a CD8 T cell co-receptor. Ligation of the TCR by a peptide-MHC complex leads to transduction of the signals necessary for activation of the T cell.

In some embodiments, Signal 1 comprises one more more exogenous polypeptides comprising an antigen-presenting polypeptide. In some embodiments, Signal 1 comprises an antigen presenting polypeptide specifically bound to (presenting) an antigenic peptide (e.g, covalently or non-covalently). In some embodiments, the antigen-presenting polypeptide is an MHC class I polypeptide, an MHC class I single chain fusion, an MHC class II polypeptide, or an MHC class II single chain fusion. In some embodiments, the MHC class I polypeptide is selected from the group consisting of HLA A, HLA B, and HLA C. In some embodiments, the MHC class II polypeptide is selected from the group consisting of HLA-DPα, HLA-DPβ, HLA-DM, HLA DOA, HLA DOB, HLA DQα, HLA DQβ, HLA DRα, and HLA DRβ.

Signal 2—Co-Stimulation

To become fully activated, T cells require a second signal in addition to TCR-mediated antigen recognition. This second signal, or co-stimulation, is important for proper T cell activation. In some embodiments, signal 2 comprises one or more exogenous costimulatory polypeptides. In some embodiments, the one or more exogenous costimulatory polypeptides is selected from the group consisting of 4-1BBL, LIGHT, anti CD28, CD80, CD86, CD70, OX40L, GITRL, TIM4, SLAM, CD48, CD58, CD83, CD155, CD112, IL-15, IL-15Rα fused to IL-15, IL-21, ICAM-1, a ligand for LFA-1, anti CD3 antibody, and any combination thereof. In some embodiments, Signal 2 comprises one or more exogenous costimulatory polypeptides selected from the group consisting of 4-1BBL, CD80, CD86, CD83, CD70, LIGHT, HVEM, CD40L, OX40L, TL1A, GITRL, and CD30L.

Signal 3—Cytokines

To induce more efficient expansion and specific differentiation of T cells, a third signal (Signal 3) can be used. In some embodiments, Signal 3 comprises one or more exogenous polypeptides comprising one or more cytokines. In some embodiments, Signal 3 comprises one or more exogenous polypeptides selected from the group consisting of IL2, IL15, IL7, IL12, IL18, IL21, IL4; IL6, IL23, IL27, IL17, IL10, TGF-beta, IFN-gamma, IL-1 beta, GM-CSF, IL-15, IL-15Rα fused to IL-15, and IL-25

In addition to immunostimulatory cytokines, immunoinhibitory cytokines are capable of dampening the immune response or can lead to tolerance. Accordingly, in some embodiments, Signal 3 comprises one or more exogenous co-inhibitory polypeptides. In some embodiments, the one or more exogenous co-inhibitory polypeptide is selected from the group consisting of IL-35, IL-10, VSIG-3 and a LAG3 agonist.

In some embodiments, the aAPC comprises at the cell surface an exogenous polypeptide comprising Signal 1.

In some embodiments, the aAPC comprises at the cell surface an exogenous polypeptide comprising Signal 1 and an exogenous polypeptide comprising Signal 2. In some embodiments, the aAPC comprises at the cell surface an exogenous polypeptide comprising Signal 1, an exogenous polypeptide comprising Signal 2, and an exogenous polypeptide comprising Signal 3. In some embodiments, the aAPC comprises at the cell surface more than one exogenous polypeptide comprising more than one Signal 1, and an exogenous polypeptide comprising Signal 2. In some embodiments, the aAPC comprises at the cell surface more than one exogenous polypeptide comprising more than one Signal 1, an exogenous polypeptide comprising Signal 2, and an exogenous polypeptide comprising Signal 3. In some embodiments, the aAPC comprises at the cell surface an exogenous polypeptide comprising Signal 1 and more than one exogenous polypeptide comprising more than one Signal 2. In some embodiments, the aAPC comprises at the cell surface an exogenous polypeptide comprising Signal 1, more than one exogenous polypeptide comprising more than one Signal 2, and an exogenous polypeptide comprising Signal 3. In some embodiments, the aAPC comprises at the cell surface an exogenous polypeptide comprising Signal 1, an exogenous polypeptide comprising Signal 2, and more than one exogenous polypeptide comprising more than one Signal 3. In some embodiments, the aAPC comprises at the cell surface more than one exogenous polypeptide comprising more than one Signal 1, an exogenous polypeptide comprising Signal 2, and more than one exogenous polypeptide comprising more than one Signal 3. In some embodiments, the aAPC comprises at the cell surface more than one exogenous polypeptide comprising more than one Signal 1, more than one exogenous polypeptide comprising more than one Signal 2, and an exogenous polypeptide comprising Signal 3. In some embodiments, the aAPC comprises at the cell surface more than one exogenous polypeptide comprising more than one Signal 1, more than one exogenous polypeptide comprising more than one Signal 2, and more than one exogenous polypeptide comprising more than one Signal 3.

In some embodiments, the aAPC comprises an exogenous polypeptide comprising Signal 1 and an exogenous polypeptide comprising Signal 2, wherein Signal 1 and Signal 2 are selected from the following combinations: MHC class I and 4-1BBL; MHC class II and 4-1BBL; MHC class I and CD80; MHC class II and CD80; MHC class I and CD86; MHC class II and CD86; MHC class I and CD83; MHC class II and CD83; MHC class I and CD70; MHC class II and CD70; MHC class I and LIGHT; MHC class II and LIGHT; MHC class I and HVEM; MHC class II and HVEM; MHC class I and CD40L; MHC class II and CD40L; MHC class I and OX40L; MHC class II and OX40L; MHC class I and TL1A; MHC class II and TL1A; MHC class I and GITRL; MHC class II and GITRL; MHC class I and CD30L; or MHC class II and CD30L.

In some embodiments, the aAPC comprises an exogenous polypeptide comprising Signal 1, an exogenous polypeptide comprising Signal 2, and an exogenous polypeptide comprising Signal 3, wherein Signal 1, Signal 2 and Signal 3 are selected from the following combinations: MHC class I, 4-1BBL, and IL2; MHC class II, 4-1BBL, and IL2; MHC class I, CD80, and IL2; MHC class II, CD80, and IL2; MHC class I, CD86, and IL2; MHC class II, CD86, and IL2; MHC class I, CD83, and IL2; MHC class II, CD83, and IL2; MHC class I, CD70, and IL2; MHC class II, CD70, and IL2; MHC class I, LIGHT, and IL2; MHC class II, LIGHT, and IL2; MHC class I, HVEM, and IL2; MHC class II, HVEM, and IL2; MHC class I, CD40L, and IL2; MHC class II, CD40L, and IL2; MHC class I, OX40L, and IL2; MHC class II, OX40L, and IL2; MHC class I, TL1A, and IL2; MHC class II, TL1A, and IL2; MHC class I, GITRL, and IL2; MHC class II, GITRL, and IL2; MHC class I, CD30L and IL2; MHC class II, CD30L and IL2; MHC class I, 4-1BBL, and IL15; MHC class II, 4-1BBL, and IL15; MHC class I, CD80, and IL15; MHC class II, CD80, and IL15; MHC class I, CD86, and IL15; MHC class II, CD86, and IL15; MHC class I, CD83, and IL15; MHC class II, CD83, and IL15; MHC class I, CD70, and IL15; MHC class II, CD70, and IL15; MHC class I, LIGHT, and IL15; MHC class II, LIGHT, and IL15; MHC class I, HVEM, and IL15; HC class II, HVEM, and IL15; MHC class I, CD40L, and IL15; MHC class II, CD40L, and IL15; MHC class I, OX40L, and IL15; MHC class II, OX40L, and IL15; MHC class I, TL1A, and IL15; MHC class II, TL1A, and IL15; MHC class I, GITRL, and IL15; MHC class II, GITRL, and IL15; MHC class I, CD30L and IL15; MHC class II, CD30L and IL15; MHC class I, 4-1BBL, and IL7; MHC class II, 4-1BBL, and IL7; MHC class I, CD80, and IL7; MHC class II, CD80, and IL7; MHC class I, CD86, and IL7; MHC class II, CD86, and IL7; MHC class I, CD83, and IL7; MHC class II, CD83, and IL7; MHC class I, CD70, and IL7; MHC class II, CD70, and IL7; MHC class I, LIGHT, and IL7; MHC class II, LIGHT, and IL7; MHC class I, HVEM, and IL7; MHC class II, HVEM, and IL7; MHC class I, CD40L, and IL7; MHC class II, CD40L, and IL7; MHC class I, OX40L, and IL7; MHC class II, OX40L, and IL7; MHC class I, TL1A, and IL7; MHC class II, TL1A, and IL7; MHC class I, GITRL, and IL7; MHC class II, GITRL, and IL7; MHC class I, CD30L and IL7; MHC class II, CD30L and IL7; MHC class I, 4-1BBL, and IL12; MHC class II, 4-1BBL, and IL12; MHC class I, CD80, and IL12; MHC class II, CD80, and IL12; MHC class I, CD86, and IL12; MHC class II, CD86, and IL12; MHC class I, CD83, and IL12; MHC class II, CD83, and IL12; MHC class I, CD70, and IL12; MHC class II, CD70, and IL12; MHC class I, LIGHT, and IL12; MHC class II, LIGHT, and IL12; MHC class I, HVEM, and IL12; MHC class II, HVEM, and IL12; MHC class I, CD40L, and IL12; MHC class II, CD40L, and IL12; MHC class I, OX40L, and IL12; MHC class II, OX40L, and IL12; MHC class I, TL1A, and IL12; MHC class II, TL1A, and IL12; MHC class I, GITRL, and IL12; MHC class II, GITRL, and IL12; MHC class I, CD30L and IL12; MHC class II, CD30L and IL12; MHC class I, 4-1BBL, and IL18; MHC class II, 4-1BBL, and IL18; MHC class I, CD80, and IL18; MHC class II, CD80, and IL18; MHC class I, CD86, and IL18; MHC class II, CD86, and IL18; MHC class I, CD83, and IL18; MHC class II, CD83, and IL18; MHC class I, CD70, and IL18; MHC class II, CD70, and IL18; MHC class I, LIGHT, and IL18; MHC class II, LIGHT, and IL18; MHC class I, HVEM, and IL18; MHC class II, HVEM, and IL18; MHC class I, CD40L, and IL18; MHC class II, CD40L, and IL18; MHC class I, OX40L, and IL18; MHC class II, OX40L, and IL18; MHC class I, TL1A, and IL18; MHC class II, TL1A, and IL18; MHC class I, GITRL, and IL18; MHC class II, GITRL, and IL18; MHC class I, CD30L and IL18; MHC class II, CD30L and IL18; MHC class I, 4-1BBL, and IL21; MHC class II, 4-1BBL, and IL21; MHC class I, CD80, and IL21; MHC class II, CD80, and IL21; MHC class I, CD86, and IL21; MHC class II, CD86, and IL21; MHC class I, CD83, and IL21; MHC class II, CD83, and IL21; MHC class I, CD70, and IL21; MHC class II, CD70, and IL21; MHC class I, LIGHT, and IL21; MHC class II, LIGHT, and IL21; MHC class I, HVEM, and IL21; MHC class II, HVEM, and IL21; MHC class I, CD40L, and IL21; MHC class II, CD40L, and IL21; MHC class I, OX40L, and IL21; MHC class II, OX40L, and IL21; MHC class I, TL1A, and IL21; MHC class II, TL1A, and IL21; MHC class I, GITRL, and IL21; MHC class II, GITRL, and IL21; MHC class I, CD30L and IL21; MHC class II, CD30L and IL21; MHC class I, 4-1BBL, and IL4; MHC class II, 4-1BBL, and IL4; MHC class I, CD80, and IL4; MHC class II, CD80, and IL4; MHC class I, CD86, and IL4; MHC class II, CD86, and IL4; MHC class I, CD83, and IL4; MHC class II, CD83, and IL4; MHC class I, CD70, and IL4; MHC class II, CD70, and IL4; MHC class I, LIGHT, and IL4; MHC class II, LIGHT, and IL4; MHC class I, HVEM, and IL4; MHC class II, HVEM, and IL4; MHC class I, CD40L, and IL4; MHC class II, CD40L, and IL4; MHC class I, OX40L, and IL4; MHC class II, OX40L, and IL4; MHC class I, TL1A, and IL4; MHC class II, TL1A, and IL4; MHC class I, GITRL, and IL4; MHC class II, GITRL, and IL4; MHC class I, CD30L and IL4; MHC class II, CD30L and IL4; MHC class I, 4-1BBL, and IL6; MHC class II, 4-1BBL, and IL6; MHC class I, CD80, and IL6; MHC class II, CD80, and IL6; MHC class I, CD86, and IL6; MHC class II, CD86, and IL6; MHC class I, CD83, and IL6; MHC class II, CD83, and IL6; MHC class I, CD70, and IL6; MHC class II, CD70, and IL6; MHC class I, LIGHT, and IL6; MHC class II, LIGHT, and IL6; MHC class I, HVEM, and IL6; MHC class II, HVEM, and IL6; MHC class I, CD40L, and IL6; MHC class II, CD40L, and IL6; MHC class I, OX40L, and IL6; MHC class II, OX40L, and IL6; MHC class I, TL1A, and IL6; MHC class II, TL1A, and IL6; MHC class I, GITRL, and IL6; MHC class II, GITRL, and IL6; MHC class I, CD30L and IL6; MHC class II, CD30L and IL6; MHC class I, 4-1BBL, and IL23; MHC class II, 4-1BBL, and IL23; MHC class I, CD80, and IL23; MHC class II, CD80, and IL23; MHC class I, CD86, and IL23; MHC class II, CD86, and IL23; MHC class I, CD83, and IL23; MHC class II, CD83, and IL23; MHC class I, CD70, and IL23; MHC class II, CD70, and IL23; MHC class I, LIGHT, and IL23; MHC class II, LIGHT, and IL23; MHC class I, HVEM, and IL23; MHC class II, HVEM, and IL23; MHC class I, CD40L, and IL23; MHC class II, CD40L, and IL23; MHC class I, OX40L, and IL23; MHC class II, OX40L, and IL23; MHC class I, TL1A, and IL23; MHC class II, TL1A, and IL23; MHC class I, GITRL, and IL23; MHC class II, GITRL, and IL23; MHC class I, CD30L and IL23; MHC class II, CD30L and IL23; MHC class I, 4-1BBL, and IL27; MHC class II, 4-1BBL, and IL27; MHC class I, CD80, and IL27; MHC class II, CD80, and IL27; MHC class I, CD86, and IL27; MHC class II, CD86, and IL27; MHC class I, CD83, and IL27; MHC class II, CD83, and IL27; MHC class I, CD70, and IL27; MHC class II, CD70, and IL27; MHC class I, LIGHT, and IL27; MHC class II, LIGHT, and IL27; MHC class I, HVEM, and IL27; MHC class II, HVEM, and IL27; MHC class I, CD40L, and IL27; MHC class II, CD40L, and IL27; MHC class I, OX40L, and IL27; MHC class II, OX40L, and IL27; MHC class I, TL1A, and IL27; MHC class II, TL1A, and IL27; MHC class I, GITRL, and IL27; MHC class II, GITRL, and IL27; MHC class I, CD30L and IL27; MHC class II, CD30L and IL27; MHC class I, 4-1BBL, and IL17; MHC class II, 4-1BBL, and IL17; MHC class I, CD80, and IL17; MHC class II, CD80, and IL17; MHC class I, CD86, and IL17; MHC class II, CD86, and IL17; MHC class I, CD83, and IL17; MHC class II, CD83, and IL17; MHC class I, CD70, and IL17; MHC class II, CD70, and IL17; MHC class I, LIGHT, and IL17; MHC class II, LIGHT, and IL17; MHC class I, HVEM, and IL17; MHC class II, HVEM, and IL17; MHC class I, CD40L, and IL17; MHC class II, CD40L, and IL17; MHC class I, OX40L, and IL17; MHC class II, OX40L, and IL17; MHC class I, TL1A, and IL17; MHC class II, TL1A, and IL17; MHC class I, GITRL, and IL17; MHC class II, GITRL, and IL17; MHC class I, CD30L and IL17; MHC class II, CD30L and IL17; MHC class I, 4-1BBL, and IL10; MHC class II, 4-1BBL, and IL10; MHC class I, CD80, and IL10; MHC class II, CD80, and IL10; MHC class I, CD86, and IL10; MHC class II, CD86, and IL10; MHC class I, CD83, and IL10; MHC class II, CD83, and IL10; MHC class I, CD70, and IL10; MHC class II, CD70, and IL10; MHC class I, LIGHT, and IL10; MHC class II, LIGHT, and IL10; MHC class I, HVEM, and IL10; MHC class II, HVEM, and IL10; MHC class I, CD40L, and IL10; MHC class II, CD40L, and IL10; MHC class I, OX40L, and IL10; MHC class II, OX40L, and IL10; MHC class I, TL1A, and IL10; MHC class II, TL1A, and IL10; MHC class I, GITRL, and IL10; MHC class II, GITRL, and IL10; MHC class I, CD30L and IL10; MHC class II, CD30L and IL10; MHC class I, 4-1BBL, and TGF-beta; MHC class II, 4-1BBL, and TGF-beta; MHC class I, CD80, and TGF-beta; MHC class II, CD80, and TGF-beta; MHC class I, CD86, and TGF-beta; MHC class II, CD86, and TGF-beta; MHC class I, CD83, and TGF-beta; MHC class II, CD83, and TGF-beta; MHC class I, CD70, and TGF-beta; MHC class II, CD70, and TGF-beta; MHC class I, LIGHT, and TGF-beta; MHC class II, LIGHT, and TGF-beta; MHC class I, HVEM, and TGF-beta; MHC class II, HVEM, and TGF-beta; MHC class I, CD40L, and TGF-beta; MHC class II, CD40L, and TGF-beta; MHC class I, OX40L, and TGF-beta; MHC class II, OX40L, and TGF-beta; MHC class I, TL1A, and TGF-beta; MHC class II, TL1A, and TGF-beta; MHC class I, GITRL, and TGF-beta; MHC class II, GITRL, and TGF-beta; MHC class I, CD30L and TGF-beta; MHC class II, CD30L and TGF-beta; MHC class I, 4-1BBL, and IFN-gamma; MHC class II, 4-1BBL, and IFN-gamma; MHC class I, CD80, and IFN-gamma; MHC class II, CD80, and IFN-gamma; MHC class I, CD86, and IFN-gamma; MHC class II, CD86, and IFN-gamma; MHC class I, CD83, and IFN-gamma; MHC class II, CD83, and IFN-gamma; MHC class I, CD70, and IFN-gamma; MHC class II, CD70, and IFN-gamma; MHC class I, LIGHT, and IFN-gamma; MHC class II, LIGHT, and IFN-gamma; MHC class I, HVEM, and IFN-gamma; MHC class II, HVEM, and IFN-gamma; MHC class I, CD40L, and IFN-gamma; MHC class II, CD40L, and IFN-gamma; MHC class I, OX40L, and IFN-gamma; MHC class II, OX40L, and IFN-gamma; MHC class I, TL1A, and IFN-gamma; MHC class II, TL1A, and IFN-gamma; MHC class I, GITRL, and IFN-gamma; MHC class II, GITRL, and IFN-gamma; MHC class I, CD30L and IFN-gamma; MHC class II, CD30L and IFN-gamma; MHC class I, 4-1BBL, and IL-1 beta; MHC class II, 4-1BBL, and IL-1 beta; MHC class I, CD80, and IL-1 beta; MHC class II, CD80, and IL-1 beta; MHC class I, CD86, and IL-1 beta; MHC class II, CD86, and IL-1 beta; MHC class I, CD83, and IL-1 beta; MHC class II, CD83, and IL-1 beta; MHC class I, CD70, and IL-1 beta; MHC class II, CD70, and IL-1 beta; MHC class I, LIGHT, and IL-1 beta; MHC class II, LIGHT, and IL-1 beta; MHC class I, HVEM, and IL-1 beta; MHC class II, HVEM, and IL-1 beta; MHC class I, CD40L, and IL-1 beta; MHC class II, CD40L, and IL-1 beta; MHC class I, OX40L, and IL-1 beta; MHC class II, OX40L, and IL-1 beta; MHC class I, TL1A, and IL-1 beta; MHC class II, TL1A, and IL-1 beta; MHC class I, GITRL, and IL-1 beta; MHC class II, GITRL, and IL-1 beta; MHC class I, CD30L and IL-1 beta; MHC class II, CD30L and IL-1 beta; MHC class I, 4-1BBL, and GM-CSF; MHC class II, 4-1BBL, and GM-CSF; MHC class I, CD80, and GM-CSF; MHC class II, CD80, and GM-CSF; MHC class I, CD86, and GM-CSF; MHC class II, CD86, and GM-CSF; MHC class I, CD83, and GM-CSF; MHC class II, CD83, and GM-CSF; MHC class I, CD70, and GM-CSF; MHC class II, CD70, and GM-CSF; MHC class I, LIGHT, and GM-CSF; MHC class II, LIGHT, and GM-CSF; MHC class I, HVEM, and GM-CSF; MHC class II, HVEM, and GM-CSF; MHC class I, CD40L, and GM-CSF; MHC class II, CD40L, and GM-CSF; MHC class I, OX40L, and GM-CSF; MHC class II, OX40L, and GM-CSF; MHC class I, TL1A, and GM-CSF; MHC class II, TL1A, and GM-CSF; MHC class I, GITRL, and GM-CSF; MHC class II, GITRL, and GM-CSF; MHC class I, CD30L and GM-CSF; MHC class II, CD30L and GM-CSF; MHC class I, 4-1BBL, and IL-25; MHC class II, 4-1BBL, and IL-25; MHC class I, CD80, and IL-25; MHC class II, CD80, and IL-25; MHC class I, CD86, and IL-25; MHC class II, CD86, and IL-25; MHC class I, CD83, and IL-25; MHC class II, CD83, and IL-25; MHC class I, CD70, and IL-25; MHC class II, CD70, and IL-25; MHC class I, LIGHT, and IL-25; MHC class II, LIGHT, and IL-25; MHC class I, HVEM, and IL-25; MHC class II, HVEM, and IL-25; MHC class I, CD40L, and IL-25; MHC class II, CD40L, and IL-25; MHC class I, OX40L, and IL-25; MHC class II, OX40L, and IL-25; MHC class I, TL1A, and IL-25; MHC class II, TL1A, and IL-25; MHC class I, GITRL, and IL-25; MHC class II, GITRL, and IL-25; MHC class I, CD30L and IL-25; or MHC class II, CD30L and IL-25.

It will be understood that for any of the foregoing combinations of Signal 1, Signal 2 and/or Signal 3, the MHC class I molecule can be any MHC class I antigen presenting polypeptide or MHC class I single chain fusion polypeptide described herein. Similarly, it will be understood that for any of the foregoing combinations of Signal 1, Signal 2 and/or Signal 3, the MHC class II molecule can be any MHC class II antigen presenting polypeptide or MHC class I single chain fusion polypeptide described herein.

### Cell Adhesion Molecules

In some embodiments, in addition to Signal 1, Signal 2 and/or Signal 3, the aAPCs described herein further comprise at the cell surface one or more exogenous polypeptides comprising cell adhesion molecules. Cell adhesion molecules further facilitate the interation between T-cells and the aAPCs. In some embodiments, the cell adhesion molecules mediate or facilitate the formation of the immunological synapse. In some embodiments, the one or more cell adhesion molecule is selected from the group consisting of ICAM4/LW, CD36, CD58/LFA3, CD47, VLA4, BCAM/Lu, CD44, CD99/MIC2, ICAM1, JAM1 and CD147, or any combination thereof.

In some embodiments, the aAPC comprises at the cell surface an exogenous polypeptide comprising Signal 1, an exogenous polypeptide comprising Signal 2, and one or more exogenous polypeptides comprising cell adhesion molecules. In some embodiments, the aAPC comprises at the cell surface an exogenous polypeptide comprising Signal 1, an exogenous polypeptide comprising Signal 2, an exogenous polypeptide comprising Signal 3, and one or more exogenous polypeptides comprising cell adhesion molecules.

In some embodiments, the aAPC comprises at the cell surface more than one exogenous polypeptide comprising more than one Signal 1, an exogenous polypeptide comprising Signal 2, and one or more exogenous polypeptides comprising cell adhesion molecules. In some embodiments, the aAPC comprises at the cell surface more than one exogenous polypeptide comprising more than one Signal 1, an exogenous polypeptide comprising Signal 2, an exogenous polypeptide comprising Signal 3, and one or more exogenous polypeptides comprising cell adhesion molecules.

In some embodiments, the aAPC comprises at the cell surface an exogenous polypeptide comprising Signal 1, more than one exogenous polypeptide comprising more than one Signal 2, and one or more exogenous polypeptides comprising cell adhesion molecules. In some embodiments, the aAPC comprises at the cell surface an exogenous polypeptide comprising Signal 1, more than one exogenous polypeptide comprising more than one Signal 2, an exogenous polypeptide comprising Signal 3, and one or more exogenous polypeptides comprising cell adhesion molecules.

In some embodiments, the aAPC comprises at the cell surface an exogenous polypeptide comprising Signal 1, an exogenous polypeptide comprising Signal 2, more than one exogenous polypeptide comprising more than one Signal 3, and one or more exogenous polypeptides comprising cell adhesion molecules.

In some embodiments, the aAPC comprises at the cell surface more than one exogenous polypeptide comprising more than one Signal 1, an exogenous polypeptide comprising Signal 2, more than one exogenous polypeptide comprising more than one Signal 3, and one or more exogenous polypeptides comprising cell adhesion molecules.

In some embodiments, the aAPC comprises at the cell surface more than one exogenous polypeptide comprising more than one Signal 1, more than one exogenous polypeptide comprising more than one Signal 2, an exogenous polypeptide comprising Signal 3, and one or more exogenous polypeptides comprising cell adhesion molecules.

In some embodiments, the aAPC comprises at the cell surface more than one exogenous polypeptide comprising more than one Signal 1, more than one exogenous polypeptide comprising more than one Signal 2, more than one exogenous polypeptide comprising more than one Signal 3, and one or more exogenous polypeptides comprising cell adhesion molecules.

In some embodiments, the one or more exogenous polypeptides comprising Signal 1, the one or more exogenous polypeptides comprising Signal 2, the one or more exogenous polypeptides comprising Signal 3, and the one or more exogenous polypeptides comprising cell adhesion molecules are selected from the exogenous polypeptides shown in Table 8.

TABLE 8
Cell Adhesion Molecules

| | |
|---|---|
| Signal 1 | MHC class I; and MHC class II |
| Signal 2 | 4-1BBL; CD80; CD86; CD83; CD70; |
| | LIGHT; HVEM; CD40L; OX40L; TL1A; |
| | GITRL; and CD30L |
| Signal 3 | IL2; IL15; IL7; IL12; IL18; IL21; IL4; IL6; |
| | IL23; IL27; IL17; IL10; TGF-beta; IFN- |
| | gamma; IL-1 beta; GM-CSF; and IL-25 |
| Adhesion | ICAM4/LW; CD36; CD58/LFA3; CD47; |
| Molecules | VLA4; BCAM/Lu; CD44; CD99/MIC2; |
| | ICAM1; JAM1 and CD147 |

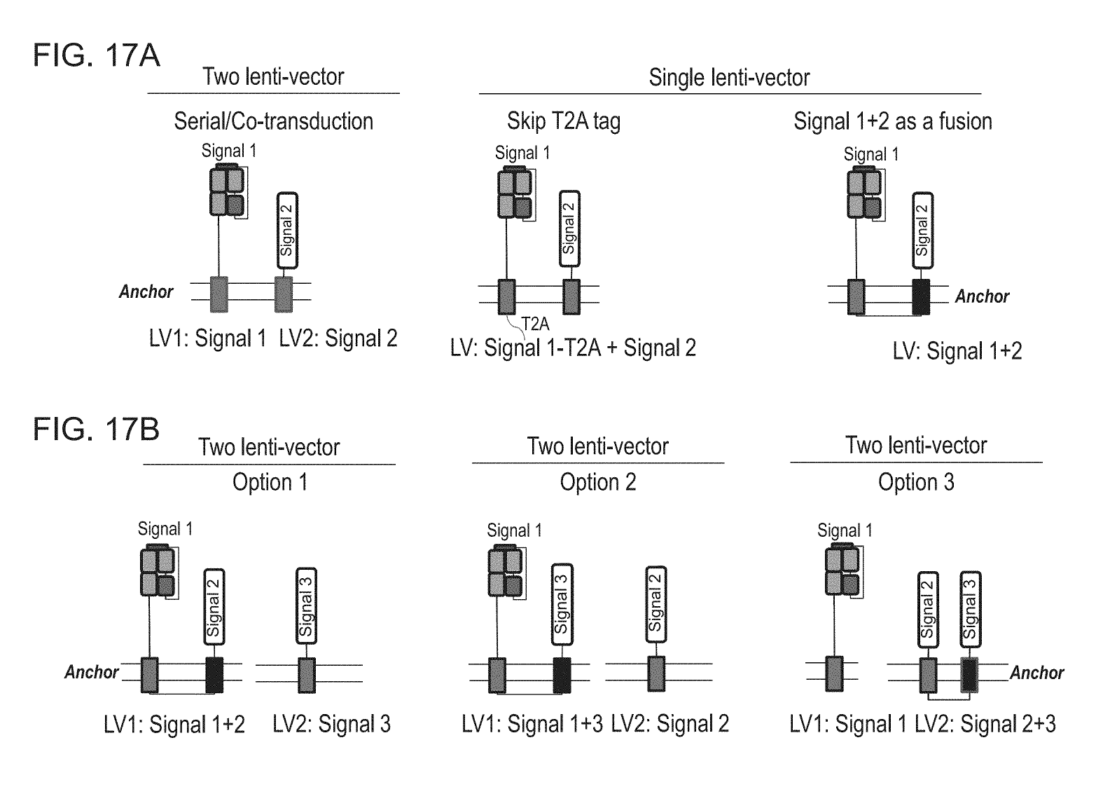

In embodiments where the aAPC comprises a polypeptide comprising Signal 1 and a polypeptide comprising Signal 2, the polypeptides can be present on the surface of the aAPC in different configurations, e.g., as shown in FIG. 17A. In some embodiments, the polypeptide comprising Signal 1 and polypeptide comprising Signal 2 are present as independent, separate polypeptides (e.g., each polypeptide comprising an anchor) and, e.g., are encoded by nucleic acids present on two separate lentiviral vectors which are used to serially transduce or co-transduce the erythroid precursor cell (two lenti-vector).

In some embodiments, the polypeptide comprising Signal 1 and polypeptide comprising Signal 2 are present as a fusion polypeptide, e.g., connected or tethered by a linker sequence, wherein each polypeptide comprises an anchor (signal 1+2 as a fusion). In these embodiments, the fusion polypeptide is encoded by a single lentiviral vector which is used to transduce the erythroid precursor cell.

In some embodiments, the polypeptide comprising Signal 1 and polypeptide comprising Signal 2 are present on the surface of the cell (e.g, each polypeptide comprising an anchor) wherein the polypeptides are separated by a viral-derived 2A element. Multiple 2A elements are known in the art and can be used as described herein, including T2A, P2A, E2A, and F2A (see, e.g., Liu et al. (2017) Sci. Rep. 7(1): 2193, incorporated in its entirety herein by reference). In some embodiments the polypeptide comprising Signal 1 and polypeptide comprising Signal 2 are separated by T2A (Skip T2A tag). In these embodiments, the polypeptides are encoded by a single lentiviral vector which is used to transduce the erythroid precursor cell.

In embodiments where the aAPC comprises a polypeptide comprising Signal 1, a polypeptide comprising Signal 2 and a polypeptide comprising Signal 3, the polypeptides can be present on the surface of the aAPC in different configurations, e.g., as shown in FIG. 17. In some embodiments, the polypeptide comprising Signal 1 and the polypeptide comprising Signal 2 are present as a fusion polypeptide, e.g., connected by a linker, and the polypeptide comprising Signal 3 is a separate polypeptide (option 1). In some embodiments, the polypeptide comprising Signal 1 and the polypeptide comprising Signal 3 are present as a fusion polypeptide, e.g., connected by a linker, and the polypeptide comprising Signal 2 is a separate polypeptide (option 2). In some embodiments, the polypeptide comprising Signal 2 and the polypeptide comprising Signal 3 are present as a fusion polypeptide, e.g., connected by a linker, and the polypeptide comprising Signal 1 is a separate polypeptide (option 3). In these embodiments, it will be understood that, when preparing the aAPCs, the fusion polypeptide (comprising Signals 1 and 2, Signals 2 and 3, or Signals 1 and 3) can be encoded by one lentiviral vector, and the separate polypeptide (Signal 1, Signal 2 or Signal 3) can be encoded by a second lentiviral vector.

In some embodiments, the tether or linker between Signal 1 and Signal 2, between Signal 1 and Signal 3, or between Signal 2 and Signal 3 is a poly-GlySer linker. In some embodiments, the tether or linker between Signal 1 and Signal 2, between Signal 1 and Signal 3, or between Signal 2 and Signal 3 is a snorkel linker.

Examples of exemplary fusion constructs comprising Signal 1 and Signal 2 are provided in Table 9 below.

TABLE 9
Constructs

| | | SEQ ID |
|---|---|---|
| Description | Amino Acid Sequence | NO: |
| Beta 2 microglobulin | MSRSVALAVLALLSLSGLEA | 730 |
| leader | | |
| HPV16 E7₁₁₋₁₉ | YMLDLQPET | 713 |
| peptide | | |
| linker between | GGGGSGGGGSGGGGS | 732 |
| peptide and Beta 2 | | |
| microglobulin | | |
| beta 2 microglobulin | IQRTPKIQVYSRHPAENGKSNFLNCYVSGFHPSDIEVDLLKNGERIEKVEHS | 839 |
| (without leader) | DLSFSKDWSFYLLYYTEFTPTEKDEYACRVNHVTLSQPKIVKWDRDM | |
| linker between Beta | GGGGSGGGGSGGGGSGGGGS | 733 |
| 2 microglobulin and | | |
| HLA-A*0201 | | |
| HLA-A*0201 Y84A | GSHSMRYFFTSVSRPGRGEPRFIAVGYVDDTQFVRFDSDAASQRMEPRAP | 840 |
| | WIEQEGPEYWDGETRKVKAHSQTHRVDLGTLRGAYNQSEAGSHTVQRM | |
| | YGCDVGSDWRFLRGYHQYAYDGKDYIALKEDLRSWTAADMAAQTTKHK | |
| | WEAAHVAEQLRAYLEGTCVEWLRRYLENGKETLQRTDAPKTHMTHHAVS | |
| | DHEATLRCWALSFYPAEITLTWQRDGEDQTQDTELVETRPAGDGTFQKW | |
| | AAVVVPSGQEQRYTCHVQHEGLPKPLTLRWEPSSQPTIPI | |
| linker between HLA- | GSGSGSGSEDGSGSGSGS | 734 |
| A*0201 and | | |
| Glycophorin A | | |
| linker between HLA- | GSGSGSGSGSGSGSGSGS | 735 |
| A*0201 and | | |
| Glycophorin A | | |
| Glycophorin A | LSTTEVAMHTSTSSSVTKSYISSQTNDTHKR DTYAATP RAHEVSEISVRTVY | 728 |
| | PPEEETGERVQLAHHFSEPEITLIIFGVMAGVIGTILLISYGIRRLIKKSPSDVK | |
| | PLPSPDTDVPLSSVEIENPETSDQ | |
| HLA-A*0201 | GSHSMRYFFTSVSRPGRGEPRFIAVGYVDDTQFVRFDSDAASQRMEPRAP | 841 |
| | WIEQEGPEYWDGETRKVKAHSQTHRVDLGTLRGYYNQSEAGSHTVQRM | |
| | YGCDVGSDWRFLRGYHQYAYDGKDYIALKEDLRSWTAADMAAQTTKHK | |
| | WEAAHVAEQLRAYLEGTCVEWLRRYLENGKETLQRTDAPKTHMTHHAVS | |
| | DHEATLRCWALSFYPAEITLTWQRDGEDQTQDTELVETRPAGDGTFQKW | |
| | AAVVVPSGQEQRYTCHVQHEGLPKPLTLRWEPSSQPTIPI | |
| HLA-A*0201 Y84C | GSHSMRYFFTSVSRPGRGEPRFIAVGYVDDTQFVRFDSDAASQRMEPRAP | 842 |
| | WIEQEGPEYWDGETRKVKAHSQTHRVDLGTLRGCYNQSEAGSHTVQRM | |
| | YGCDVGSDWRFLRGYHQYAYDGKDYIALKEDLRSWTAADMAAQTTKHK | |
| | WEAAHVAEQLRAYLEGTCVEWLRRYLENGKETLQRTDAPKTHMTHHAVS | |
| | DHEATLRCWALSFYPAEITLTWQRDGEDQTQDTELVETRPAGDGTFQKW | |
| | AAVVVPSGQEQRYTCHVQHEGLPKPLTLRWEPSSQPTIPI | |
| linker between | GCGGSGGGGSGGGGS | 736 |
| peptide and Beta 2 | | |
| microglobulin | | |
| Fusion polypeptide | MSRSVALAVLALLSLSGLEAYMLDLQPETGGGGSGGGGSGGGGSIQRTPK | 843 |
| comprising Beta 2 | IQVYSRHPAENGKSNFLNCYVSGFHPSDIEVDLLKNGERIEKVEHSDLSFSK | |
| microglobulin leader | DWSFYLLYYTEFTPTEKDEYACRVNHVTLSQPKIVKWDRDMGGGGSGGG | |
| sequence (B2ML)- | GSGGGGSGGGGSGSHSMRYFFTSVSRPGRGEPRFIAVGYVDDTQFVRFD | |
| HPV16 E7₁₁₋₁₉ | SDAASQRMEPRAPWIEQEGPEYWDGETRKVKAHSQTHRVDLGTLRGYY | |
| peptide-Beta 2 | NQSEAGSHTVQRMYGCDVGSDWRFLRGYHQYAYDGKDYIALKEDLRSW | |
| microglobulin (B2M)- | TAADMAAQTTKHKWEAAHVAEQLRAYLEGTCVEWLRRYLENGKETLQR | |
| HLA-A*02:01 | TDAPKTHMTHHAVSDHEATLRCWALSFYPAEITLTWQRDGEDQTQDTEL | |
| | VETRPAGDGTFQKWAAVVVPSGQEQRYTCHVQHEGLPKPLTLRWEPSS | |
| | QPTIPIGSGSGSGSEDGSGSGSGS | |
| Fusion polypeptide | MSRSVALAVLALLSLSGLEAYMLDLQPETGGGGSGGGGSGGGGSIQRTPK | 844 |
| comprising B2ML- | IQVYSRHPAENGKSNFLNCYVSGFHPSDIEVDLLKNGERIEKVEHSDLSFSK | |
| HPV16 E7₁₁₋₁₉ | DWSFYLLYYTEFTPTEKDEYACRVNHVTLSQPKIVKWDRDMGGGGSGGG | |
| peptide-B2M- | GSGGGGSGGGGSGSHSMRYFFTSVSRPGRGEPRFIAVGYVDDTQFVRFD | |
| HLA-A*02:01 Y84A | SDAASQRMEPRAPWIEQEGPEYWDGETRKVKAHSQTHRVDLGTLRGAY | |
| | NQSEAGSHTVQRMYGCDVGSDWRFLRGYHQYAYDGKDYIALKEDLRSW | |
| | TAADMAAQTTKHKWEAAHVAEQLRAYLEGTCVEWLRRYLENGKETLQR | |
| | TDAPKTHMTHHAVSDHEATLRCWALSFYPAEITLTWQRDGEDQTQDTEL | |
| | VETRPAGDGTFQKWAAVVVPSGQEQRYTCHVQHEGLPKPLTLRWEPSS | |
| | QPTIPIGSGSGSGSEDGSGSGSGS | |
| Fusion polypeptide | MSRSVALAVLALLSLSGLEAYMLDLQPETGCGGSGGGGSGGGGSIQRTPK | 845 |
| comprising B2ML- | IQVYSRHPAENGKSNFLNCYVSGFHPSDIEVDLLKNGERIEKVEHSDLSFSK | |
| HPV16 E7₁₁₋₁₉ | DWSFYLLYYTEFTPTEKDEYACRVNHVTLSQPKIVKWDRDMGGGGSGGG | |
| peptide-B2M- | GSGGGGSGGGGSGSHSMRYFFTSVSRPGRGEPRFIAVGYVDDTQFVRFD | |
| HLA-A*02:01 Y84C | SDAASQRMEPRAPWIEQEGPEYWDGETRKVKAHSQTHRVDLGTLRGCY | |
| | NQSEAGSHTVQRMYGCDVGSDWRFLRGYHQYAYDGKDYIALKEDLRSW | |
| | TAADMAAQTTKHKWEAAHVAEQLRAYLEGTCVEWLRRYLENGKETLQR | |
| | TDAPKTHMTHHAVSDHEATLRCWALSFYPAEITLTWQRDGEDQTQDTEL | |
| | VETRPAGDGTFQKWAAVVVPSGQEQRYTCHVQHEGLPKPLTLRWEPSS | |
| | QPTIPIGSGSGSGSEDGSGSGSGS | |
| Fusion polypeptide | MSRSVALAVLALLSLSGLEAYMLDLQPETGGGGSGGGGSGGGGSIQRTPK | 726 |
| comprising B2ML- | IQVYSRHPAENGKSNFLNCYVSGFHPSDIEVDLLKNGERIEKVEHSDLSFSK | |
| HPV16 E711₁₁₋₁₉ | DWSFYLLYYTEFTPTEKDEYACRVNHVTLSQPKIVKWDRDMGGGGSGGG | |
| peptide-B2M- | GSGGGGSGGGGSGSHSMRYFFTSVSRPGRGEPRFIAVGYVDDTQFVRFD | |
| HLA-A*02:01- | SDAASQRMEPRAPWIEQEGPEYWDGETRKVKAHSQTHRVDLGTLRGYY | |
| Glycophorin A (GPA) | NQSEAGSHTVQRMYGCDVGSDWRFLRGYHQYAYDGKDYIALKEDLRSW | |
| | TAADMAAQTTKHKWEAAHVAEQLRAYLEGTCVEWLRRYLENGKETLQR | |
| | TDAPKTHMTHHAVSDHEATLRCWALSFYPAEITLTWQRDGEDQTQDTEL | |
| | VETRPAGDGTFQKWAAVVVPSGQEQRYTCHVQHEGLPKPLTLRWEPSS | |
| | QPTIPIGSGSGSGSEDGSGSGSGSLSTTEVAMHTSTSSSVTKSYISSQTNDT | |
| | HKRDTYAATPRAHEVSEISVRTVYPPEEETGERVQLAHHFSEPEITLIIFGVM | |
| | AGVIGTILLISYGIRRLIKKSPSDVKPLPSPDTDVPLSSVEIENPETSDQ | |
| Fusion polypeptide | MSRSVALAVLALLSLSGLEAYMLDLQPETGGGGSGGGGSGGGGSIQRTPK | 846 |
| comprising B2ML- | IQVYSRHPAENGKSNFLNCYVSGFHPSDIEVDLLKNGERIEKVEHSDLSFSK | |
| HPV16 E7₁₁₋₁₉ | DWSFYLLYYTEFTPTEKDEYACRVNHVTLSQPKIVKWDRDMGGGGSGGG | |
| peptide-B2M- | GSGGGGSGGGGSGSHSMRYFFTSVSRPGRGEPRFIAVGYVDDTQFVRFD | |
| HLA-A*02:01 Y84A- | SDAASQRMEPRAPWIEQEGPEYWDGETRKVKAHSQTHRVDLGTLRGAY | |
| GPA | NQSEAGSHTVQRMYGCDVGSDWRFLRGYHQYAYDGKDYIALKEDLRSW | |
| | TAADMAAQTTKHKWEAAHVAEQLRAYLEGTCVEWLRRYLENGKETLQR | |
| | TDAPKTHMTHHAVSDHEATLRCWALSFYPAEITLTWQRDGEDQTQDTEL | |
| | VETRPAGDGTFQKWAAVVVPSGQEQRYTCHVQHEGLPKPLTLRWEPSS | |
| | QPTIPIGSGSGSGSEDGSGSGSGSLSTTEVAMHTSTSSSVTKSYISSQTNDT | |
| | HKRDTYAATPRAHEVSEISVRTVYPPEEETGERVQLAHHFSEPEITLIIFGVM | |
| | AGVIGTILLISYGIRRLIKKSPSDVKPLPSPDTDVPLSSVEIENPETSDQ | |
| Fusion polypeptide | MSRSVALAVLALLSLSGLEAYMLDLQPETGCGGSGGGGSGGGGSIQRTPK | 847 |
| comprising B2ML- | IQVYSRHPAENGKSNFLNCYVSGFHPSDIEVDLLKNGERIEKVEHSDLSFSK | |
| HPV16 E711₁₁₋₁₉ | DWSFYLLYYTEFTPTEKDEYACRVNHVTLSQPKIVKWDRDMGGGGSGGG | |
| peptide-B2M- | GSGGGGSGGGGSGSHSMRYFFTSVSRPGRGEPRFIAVGYVDDTQFVRFD | |
| HLA-A*02:01 Y84C- | SDAASQRMEPRAPWIEQEGPEYWDGETRKVKAHSQTHRVDLGTLRGCY | |
| GPA | NQSEAGSHTVQRMYGCDVGSDWRFLRGYHQYAYDGKDYIALKEDLRSW | |
| | TAADMAAQTTKHKWEAAHVAEQLRAYLEGTCVEWLRRYLENGKETLQR | |
| | TDAPKTHMTHHAVSDHEATLRCWALSFYPAEITLTWQRDGEDQTQDTEL | |
| | VETRPAGDGTFQKWAAVVVPSGQEQRYTCHVQHEGLPKPLTLRWEPSS | |
| | QPTIPIGSGSGSGSEDGSGSGSGSLSTTEVAMHTSTSSSVTKSYISSQTNDT | |
| | HKRDTYAATPRAHEVSEISVRTVYPPEEETGERVQLAHHFSEPEITLIIFGVM | |
| | AGVIGTILLISYGIRRLIKKSPSDVKPLPSPDTDVPLSSVEIENPETSDQ | |
| Fusion polypeptide comprising beta 2 microglobulin leader (B2ML)-HPV16 E7₁₁₋₁₉ | | |
| peptide-linker-beta 2 microglobulin (B2M)-linker-HLA-A*02:01 Y84A-linker-GPA- | | |
| T2A-GPA signal peptide-4-1BBL-linker v17-GPA | | |
| Beta 2 | MSRSVALAVLALLSLSGLEAYMLDLQPETGGGGSGGGGSGGGGSIQRTPKIQ | 848 |
| microglobulin | VYSRHPAENGKSNFLNCYVSGFHPSDIEVDLLKNGERIEKVEHSDLSFSKDWSF | |
| leader (B2ML)- | YLLYYTEFTPTEKDEYACRVNHVTLSQPKIVKWDRDMGGGGSGGGGSGGGG | |
| HPV16 E711₁₁₋₁₉ | SGGGGSGSHSMRYFFTSVSRPGRGEPRFIAVGYVDDTQFVRFDSDAASQRM | |
| peptide-linker- | EPRAPWIEQEGPEYWDGETRKVKAHSQTHRVDLGTLRGAYNQSEAGSHTV | |
| Beta 2 | QRMYGCDVGSDWRFLRGYHQYAYDGKDYIALKEDLRSWTAADMAAQTTK | |
| microglobulin | HKWEAAHVAEQLRAYLEGTCVEWLRRYLENGKETLQRTDAPKTHMTHHAV | |
| (B2M)-linker- | SDHEATLRCWALSFYPAEITLTWQRDGEDQTQDTELVETRPAGDGTFQKWA | |
| HLA-A*02:01 | AVVVPSGQEQRYTCHVQHEGLPKPLTLRWEPSSQPTIPIGSGSGSGSEDGSGS | |
| Y84A-linker- | GSGSLSTTEVAMHTSTSSSVTKSYISSQTNDTHKRDTYAATPRAHEVSEISVRT | |
| GPA-T2A-GPA | VYPPEEETGERVQLAHHFSEPEITLIIFGVMAGVIGTILLISYGIRRLIKKSPSDVK | |
| signal peptide- | PLPSPDTDVPLSSVEIENPETSDQGSGEGRGSLLTCGDVEENPGPMYGKIIFVL | |
| N-terminal | LLSEIVSISAACPWAVSGARASPGSAASPRLREGPELSPDDPAGLLDLRQGMF | |
| truncated 4-1BBL | AQLVAQNVLLIDGPLSWYSDPGLAGVSLTGGLSYKEDTKELVVAKAGVYYVFF | |
| linker v17- | QLELRRVVAGEGSGSVSLALHLQPLRSAAGAAALALTVDLPPASSEARNSAFG | |
| GPA | FQGRLLHLSAGQRLGVHLHTEARARHAWQLTQGATVLGLFRVTPEIPAGLPS | |
| | PRSEGGSGGSGGGPEDEPGSGSGGGSGGGSLSTTEVAMHTSTSSSVTKSYISS | |
| | QTNDTHKRDTYAATPRAHEVSEISVRTVYPPEEETGERVQLAHHFSEPEITLIIF | |
| | GVMAGVIGTILLISYGIRRLIKKSPSDVKPLPSPDTDVPLSSVEIENPETSDQ | |
| Beta 2 | MSRSVALAVLALLSLSGLEAYMLDLQPETGGGGSGGGGSGGGGSIQRTPKIQ | 849 |
| Microglobulin | VYSRHPAENGKSNFLNCYVSGFHPSDIEVDLLKNGERIEKVEHSDLSFSKDWSF | |
| leader (B2ML)- | YLLYYTEFTPTEKDEYACRVNHVTLSQPKIVKWDRDMGGGGSGGGGSGGGG | |
| HPV16 E7₁₁₋₁₉ | SGGGGSGSHSMRYFFTSVSRPGRGEPRFIAVGYVDDTQFVRFDSDAASQRM | |
| peptide-linker- | EPRAPWIEQEGPEYWDGETRKVKAHSQTHRVDLGTLRGAYNQSEAGSHTV | |
| Beta 2 | QRMYGCDVGSDWRFLRGYHQYAYDGKDYIALKEDLRSWTAADMAAQTTK | |
| microglobulin | HKWEAAHVAEQLRAYLEGTCVEWLRRYLENGKETLQRTDAPKTHMTHHAV | |
| (B2M)-linker- | SDHEATLRCWALSFYPAEITLTWQRDGEDQTQDTELVETRPAGDGTFQKWA | |
| HLA-A*02:01 | AVVVPSGQEQRYTCHVQHEGLPKPLTLRWEPSSQPTIPIGSGSGSGSEDGSGS | |
| Y84A-linker- | GSGSLSTTEVAMHTSTSSSVTKSYISSQTNDTHKRDTYAATPRAHEVSEISVRT | |
| GPA-T2A | VYPPEEETGERVQLAHHFSEPEITLIIFGVMAGVIGTILLISYGIRRLIKKSPSDVK | |
| | PLPSPDTDVPLSSVEIENPETSDQGSGEGRGSLLTCGDVEENPG | |
| GPA signal | PMYGKIIFVLLLSEIVSISAACPWAVSGARASPGSAASPRLREGPELSPDDPAGL | 850 |
| peptide-4-1BBL | LDLRQGMFAQLVAQNVLLIDGPLSWYSDPGLAGVSLTGGLSYKEDTKELVVA | |
| linker v17- | KAGVYYVFFQLELRRVVAGEGSGSVSLALHLQPLRSAAGAAALALTVDLPPAS | |
| GPA | SEARNSAFGFQGRLLHLSAGQRLGVHLHTEARARHAWQLTQGATVLGLFRV | |
| | TPEIPAGLPSPRSEGGSGGSGGGPEDEPGSGSGGGSGGGSLSTTEVAMHTST | |
| | SSSVTKSYISSQTNDTHKRDTYAATPRAHEVSEISVRTVYPPEEETGERVQLAH | |
| | HFSEPEITLIIFGVMAGVIGTILLISYGIRRLIKKSPSDVKPLPSPDTDVPLSSVEIE | |
| | NPETSDQ | |
| GPA signal | MYGKIIFVLLLSEIVSISA | 731 |
| peptide | | |
| N-terminal- | ACPWAVSGARASPGSAASPRLREGPELSPDDPAGLLDLRQGMFAQLVAQNV | 851 |
| truncated 4-1BBL | LLIDGPLSWYSDPGLAGVSLTGGLSYKEDTKELVVAKAGVYYVFFQLELRRVVA | |
| | GEGSGSVSLALHLQPLRSAAGAAALALTVDLPPASSEARNSAFGFQGRLLHLS | |
| | AGQRLGVHLHTEARARHAWQLTQGATVLGLFRVTPEIPAGLPSPRSE | |
| linker_v17 | GGSGGSGGGPEDEPGSGSGGGSGGGS | 852 |
| GPA | LSTTEVAMHTSTSSSVTKSYISSQTNDTHKRDTYAATPRAHEVSEISVRTVYPPE | 728 |
| | EETGERVQLAHHFSEPEITLIIFGVMAGVIGTILLISYGIRRLIKKSPSDVKPLPSP | |
| | DTDVPLSSVEIENPETSDQ | |
| T2A | GSGEGRGSLLTCGDVEENPGP | 853 |
| Fusion polypeptide comprising GPA signal peptide-N-terminal truncated 4-1BBL-linker- | | |
| GPA-T2A-Beta 2 microglobulin leader (B2ML)-HPV16 E711-19 peptide-linker- | | |
| beta 2 microglobulin-linker-HLA-A*02:01 Y84A-linker-GPA | | |
| GPA signal peptide- | MYGKIIFVLLLSEIVSISAACPWAVSGARASPGSAASPRLREGPELSPDDPAG | 854 |
| N-terminal | LLDLRQGMFAQLVAQNVLLIDGPLSWYSDPGLAGVSLTGGLSYKEDTKELV | |
| truncated 4-1BBL- | VAKAGVYYVFFQLELRRVVAGEGSGSVSLALHLQPLRSAAGAAALALTVDLP | |
| linker-GPA-T2A- | PASSEARNSAFGFQGRLLHLSAGQRLGVHLHTEARARHAWQLTQGATVLG | |
| Beta 2 | LFRVTPEIPAGLPSPRSEGGSGGSGGGPEDEPGSGSGGGSGGGSLSTTEVA | |
| microglobulin | MHTSTSSSVTKSYISSQTNDTHKRDTYAATPRAHEVSEISVRTVYPPEEETGE | |
| leader (B2M14- | RVQLAHHFSEPEITLIIFGVMAGVIGTILLISYGIRRLIKKSPSDVKPLPSPDTDV | |
| HPV16 E7₁₁₋₁₉ | PLSSVEIENPETSDQGSGEGRGSLLTCGDVEENPGPMSRSVALAVLALLSLS | |
| peptide-linker- | GLEAYMLDLQPETGGGGSGGGGSGGGGSIQRTPKIQVYSRHPAENGKSNF | |
| beta 2 | LNCYVSGFHPSDIEVDLLKNGERIEKVEHSDLSFSKDWSFYLLYYTEFTPTEKD | |
| microglobulin- | EYACRVNHVTLSQPKIVKWDRDMGGGGSGGGGSGGGGSGGGGSGSHS | |
| linker-HLA-A*02:01 | MRYFFTSVSRPGRGEPRFIAVGYVDDTQFVRFDSDAASQRMEPRAPWIEQ | |
| Y84A-linker-GPA | EGPEYWDGETRKVKAHSQTHRVDLGTLRGAYNQSEAGSHWQRMYGCDV | |
| | GSDWRFLRGYHQYAYDGKDYIALKEDLRSWTAADMAAQTTKHKWEAAH | |
| | VAEQLRAYLEGTCVEWLRRYLENGKETLQRTDAPKTHMTHHAVSDHEATL | |
| | RCWALSFYPAEITLTWQRDGEDQTQDTELVETRPAGDGTFQKWAAVVVP | |
| | SGQEQRYTCHVQHEGLPKPLTLRWEPSSQPTIPIGSGSGSGSEDGSGSGSGS | |
| | LSTTEVAMHTSTSSSVTKSYISSQTNDTHKRDTYAATPRAHEVSEISVRTVYP | |
| | PEEETGERVQLAHHFSEPEITLIIFGVMAGVIGTILLISYGIRRLIKKSPSDVKPL | |
| | PSPDTDVPLSSVEIENPETSDQ | |
| GPA signal peptide- | MYGKIIFVLLLSEIVSISAACPWAVSGARASPGSAASPRLREGPELSPDDPAG | 855 |
| N-terminal | LLDLRQGMFAQLVAQNVLLIDGPLSWYSDPGLAGVSLTGGLSYKEDTKELV | |
| truncated 4-1BBL- | VAKAGVYYVFFQLELRRVVAGEGSGSVSLALHLQPLRSAAGAAALALTVDLP | |
| linker-GPA-T2A | PASSEARNSAFGFQGRLLHLSAGQRLGVHLHTEARARHAWQLTQGATVLG | |
| | LFRVTPEIPAGLPSPRSEGGSGGSGGGPEDEPGSGSGGGSGGGSLSTTEVA | |
| | MHTSTSSSVTKSYISSQTNDTHKRDTYAATPRAHEVSEISVRTVYPPEEETGE | |
| | RVQLAHHFSEPEITLIIFGVMAGVIGTILLISYGIRRLIKKSPSDVKPLPSPDTDV | |
| | PLSSVEIENPETSDQGSGEGRGSLLTCGDVEENPG | |
| T2A-Beta 2 | PMSRSVALAVLALLSLSGLEAYMLDLQPETGGGGSGGGGSGGGGSIQRTPK | 856 |
| microglobulin | IQVYSRHPAENGKSNFLNCYVSGFHPSDIEVDLLKNGERIEKVEHSDLSFSKD | |
| leader (B2M14- | WSFYLLYYTEFTPTEKDEYACRVNHVTLSQPKIVKWDRDMGGGGSGGGGS | |
| HPV16 E7₁₁₋₁₉ | GGGGSGGGGSGSHSMRYFFTSVSRPGRGEPRFIAVGYVDDTQFVRFDSDA | |
| peptide-linker- | ASQRMEPRAPWIEQEGPEYWDGETRKVKAHSQTHRVDLGTLRGAYNQSE | |
| beta 2 | AGSHTVQRMYGCDVGSDWRFLRGYHQYAYDGKDYIALKEDLRSWTAAD | |
| microglobulin- | MAAQTTKHKWEAAHVAEQLRAYLEGTCVEWLRRYLENGKETLQRTDAPK | |
| | THMTHHAVSDHEATLRCWALSFYPAEITLTWQRDGEDQTQDTELVETRPA | |
| linker-HLA-A*02:01 | GDGTFQKWAAVVVPSGQEQRYTCHVQHEGLPKPLTLRWEPSSQPTIPIGS | |
| Y84A-linker-GPA | GSGSGSEDGSGSGSGSLSTTEVAMHTSTSSSVTKSYISSQTNDTHKRDTYAA | |
| | TPRAHISVRTVYPPEEETGERVQLAHHFSEPEITLIIFGVMAGVIGTILLI | |
| | EVSESYGIRRLIKKSPSDVKPLPSPDTDVPLSSVEIENPETSDQ | |

Fusion polypeptide comprising beta 2 microglobulin leader (B2ML)-HPV16
E7₁₁₋₁₉ peptide-linker-beta 2 microglobulin (B2M)-linker-HLA-A*02:01
Y84A-linker-GPA-linker-full length 4-1BBL

| | | |
|---|---|---|
| Beta 2 | MSRSVALAVLALLSLSGLEAYMLDLQPETGGGGSGGGGSGGGGSIQRTPKI | 857 |
| microglobulin | QVYSRHPAENGKSNFLNCYVSGFHPSDIEVDLLKNGERIEKVEHSDLSFSKD | |
| leader (B2ML)- | WSFYLLYYTEFTPTEKDEYACRVNHVTLSQPKIVKWDRDMGGGGSGGGGS | |
| HPV16 E71149 | GGGGSGGGGSGSHSMRYFFTSVSRPGRGEPRFIAVGYVDDTQFVRFDSDA | |
| peptide-linker- | ASQRMEPRAPWIEQEGPEYWDGETRKVKAHSQTHRVDLGTLRGAYNQSE | |
| beta 2 | AGSHTVQRMYGCDVGSDWRFLRGYHQYAYDGKDYIALKEDLRSWTAAD | |
| microglobulin (B2M)- | MAAQTTKHKWEAAHVAEQLRAYLEGTCVEWLRRYLENGKETLQRTDAPK | |
| linker-HLA- | THMTHHAVSDHEATLRCWALSFYPAEITLTWQRDGEDQTQDTELVETRPA | |
| A*02:01 Y84A- | GDGTFQKWAAVVVPSGQEQRYTCHVQHEGLPKPLTLRWEPSSQPTIPIGS | |
| linker-GPA-linker- | GSGSGSEDGSGSGSGSLSTTEVAMHTSTSSSVTKSYISSQTNDTHKRDTYAA | |
| full length 4-1BBL | TPRAHEVSEISVRTVYPPEEETGERVQLAHHFSEPEITLIIFGVMAGVIGTILLI | |
| | SYGIRRLIKKSPSDVKPLPSPDTDVPLSSVEIENPETSDQSGRGGGGSGGGGS | |
| | GGGGSGGGGSSPAMEYASDASLDPEAPWPPAPRARACRVLPWALVAGLL | |
| | LLLLLAAACAVFLACPWAVSGARASPGSAASPRLREGPELSPDDPAGLLDLR | |
| | QGMFAQLVAQNVLLIDGPLSWYSDPGLAGVSLTGGLSYKE DTKELVVAKA | |
| | GVYYVFFQLELRRVVAGEGSGSVSLALHLQPLRSAAGAAALALTVDLPPASS | |
| | EARNSAFGFQGRLLHLSAGQRLGVHLHTEARARHAWQLTQGATVLGLFRV | |
| | TPEIPAGLPSPRSE | |
| linker | SGRGGGGSGGGGSGGGGSGGGGSSPA | 738 |
| Full length 4-1BBL | MEYASDASLDPEAPWPPAPRARACRVLPWALVAGLLLLLLLAAACAVFLAC | 858 |
| | PWAVSGARASPGSAASPRLREGPELSPDDPAGLLDLRQGMFAQLVAQNVL | |
| | LIDGPLSWYSDPGLAGVSLTGGLSYKEDTKELVVAKAGVYYVFFQLELRRVV | |
| | AGEGSGSVSLALHLQPLRSAAGAAALALTVDLPPASSEARNSAFGFQGRLLH | |
| | LSAGQRLGVHLHTEARARHAWQLTQGATVLGLFRVTPEIPAGLPSPRSE | |

Fusion polypeptide comprising beta 2 microglobulin leader (B2ML)-HPV16 E7₁₁₋₁₉
peptide-linker-beta 2 microglobulin (B2M)-linker-HLA-A*02:01 Y84A-linker-GPA-snorkel
linker-linker-N-terminal truncated 4-1BBL

| | | |
|---|---|---|
| Beta 2 | MSRSVALAVLALLSLSGLEAYMLDLQPETGGGGSGGGGSGGGGSIQRTPKI | 859 |
| microglobulin | QVYSRHPAENGKSNFLNCYVSGFHPSDIEVDLLKNGERIEKVEHSDLSFSKD | |
| leader (B2ML)- | WSFYLLYYTEFTPTEKDEYACRVNHVTLSQPKIVKWDRDMGGGGSGGGGS | |
| HPV16 E71149 | GGGGSGGGGSGSHSMRYFFTSVSRPGRGEPRFIAVGYVDDTQFVRFDSDA | |
| peptide-linker- | ASQRMEPRAPWIEQEGPEYWDGETRKVKAHSQTHRVDLGTLRGAYNQSE | |
| beta 2 | AGSHTVQRMYGCDVGSDWRFLRGYHQYAYDGKDYIALKEDLRSWTAAD | |
| microglobulin (B2M)- | MAAQTTKHKWEAAHVAEQLRAYLEGTCVEWLRRYLENGKETLQRTDAPK | |
| linker-HLA- | THMTHHAVSDHEATLRCWALSFYPAEITLTWQRDGEDQTQDTELVETRPA | |
| A*02:01 Y84A- | GDGTFQKWAAVVVPSGQEQRYTCHVQHEGLPKPLTLRWEPSSQPTIPIGS | |
| linker-GPA- | GSGSGSEDGSGSGSGSLSTTEVAMHTSTSSSVTKSYISSQTNDTHKRDTYAA | |
| snorkel linker- | TPRAHEVSEISVRTVYPPEEETGERVQLAHHFSEPEITLIIFGVMAGVIGTILLI | |
| linker-N-terminal | SYGIRRLIKKSPSDVKPLPSPDTDVPLSSVEIENPETSDQSGRGASSGSSGSGS | |
| truncated 4-1BBL | QKKPRYEIRWKVVVISAILALVVLTVISLIILIMLWGSGMQSPAGGSGGSGG | |
| | GGGSGGGSGGGSGGGSACPWAVSGARASPGSAASPRLREGPELSPDDPA | |
| | GLLDLRQGMFAQLVAQNVLLIDGPLSWYSDPGLAGVSLTGGLSYKEDTKEL | |
| | VVAKAGVYYVFFQLELRRVVAGEGSGSVSLALHLQPLRSAAGAAALALTVDL | |
| | PPASSEARNSAFGFQGRLLHLSAGQRLGVHLHTEARARHAWQLTQGATVL | |
| | GLFRVTPEIPAGLPSPRSE | |
| snorkel linker | SGRGASSGSSGSGSQKKPRYEIRWKVVVISAILALVVLTVISLIILIMLWGSGM | 740 |
| | QSPA | |
| linker between | GGSGGSGGGGGSGGGSGGGSGGGS | 737 |
| snorkel linker and | | |
| N-terminal | | |
| truncated 4-1BBL | | |

Fusion polypeptide comprising beta 2 microglobulin leader (B2ML)-HPV16 E7₁₁₋₁₉
peptide-linker-beta 2 microglobulin (B2M)-linker-HLA-A*02:01 Y84A-linker-GPA-linker-
SMIM1-linker-IL12p40-linker-IL12p35

| | | |
|---|---|---|
| Beta 2 | MSRSVALAVLALLSLSGLEAYMLDLQPETGGGGSGGGGSGGGGSIQRTPKI | 860 |
| microglobulin | QVYSRHPAENGKSNFLNCYVSGFHPSDIEVDLLKNGERIEKVEHSDLSFSKD | |
| leader (B2ML)- | WSFYLLYYTEFTPTEKDEYACRVNHVTLSQPKIVKWDRDMGGGGSGGGGS | |
| HPV16 E7₁₁₋₁₉ | GGGGSGGGGSGSHSMRYFFTSVSRPGRGEPRFIAVGYVDDTQFVRFDSDA | |
| peptide-linker- | ASQRMEPRAPWIEQEGPEYWDGETRKVKAHSQTHRVDLGTLRGAYNQSE | |
| beta 2 | AGSHTVQRMYGCDVGSDWRFLRGYHQYAYDGKDYIALKEDLRSWTAAD | |
| microglobulin (B2M)- | MAAQTTKHKWEAAHVAEQLRAYLEGTCVEWLRRYLENGKETLQRTDAPK | |
| linker-HLA- | THMTHHAVSDHEATLRCWALSFYPAEITLTWQRDGEDQTQDTELVETRPA | |
| A*02:01 Y84A- | GDGTFQKWAAVVVPSGQEQRYTCHVQHEGLPKPLTLRWEPSSQPTIPIGS | |
| linker-GPA-linker- | GSGSGSEDGSGSGSGSLSTTEVAMHTSTSSSVTKSYISSQTNDTHKRDTYAA | |
| SMIM1-linker- | TPRAHEVSEISVRTVYPPEEETGERVQLAHHFSEPEITLIIFGVMAGVIGTILLI | |
| IL12p40-linker- | SYGIRRLIKKSPSDVKPLPSPDTDVPLSSVEIENPETSDQGGGGSGGGGSGG | |
| IL12p35 | GGSGGGGSGGGGMQPQESHVHYSRWEDGSRDGVSLGAVSSTEEASRCR | |
| | RISQRLCTGKLGIAMKVLGGVALFWIIFILGYLTGYYVHKCKGGGGSGGGGS | |
| | GGGGSIWELKKDVYVVELDWYPDAPGEMVVLTCDTPEEDGITWTLDQSSE | |
| | VLGSGKTLTIQVKEFGDAGQYTCHKGGEVLSHSLLLLHKKEDGIWSTDILKD | |
| | QKEPKNKTFLRCEAKNYSGRFTCWWLTTISTDLTFSVKSSRGSSDPQGVTCG | |
| | AATLSAERVRGDNKEYEYSVECQEDSACPAAEESLPIEVMVDAVHKLKYENY | |
| | TSSFFIRDIIKPDPPKNLQLKPLKNSRQVEVSWEYPDTWSTPHSYFSLTFCVQ | |
| | VQGKSKREKKDRVFTDKTSATVICRKNASISVRAQDRYYSSSWSEWASVPC | |
| | SGGGGSGGGGSGGGGSRNLPVATPDPGMFPCLHHSQNLLRAVSNMLQK | |
| | ARQTLEFYPCTSEEIDHEDITKDKTSTVEACLPLELTKNESCLNSRETSFITNGS | |
| | CLASRKTSFMMALCLSSIYEDLKMYQVEFKTMNAKLLMDPKRQIFLDQNM | |
| | LAVIDELMQALNFNSETVPQKSSLEEPDFYKTKIKLCILLHAFRIRAVTIDRVM | |
| | SYLNAS | |
| linker between GPA | GGGGSGGGGSGGGGSGGGGSGGGG | 739 |
| and SMIM1 | | |
| SMIM1 | MQPQESHVHYSRWEDGSRDGVSLGAVSSTEEASRCRRISQRLCTGKLGIA | 729 |
| | MKVLGGVALFWIIFILGYLTGYYVHKCK | |
| linker between | GGGGSGGGGSGGGGS | 732 |
| SMIM1 and IL12p40 | | |
| IL12p40 (without | IWELKKDVYVVELDWYPDAPGEMVVLTCDTPEEDGITWTLDQSSEVLGSG | 861 |
| leader) | KTLTIQVKEFGDAGQYTCHKGGEVLSHSLLLLHKKEDGIWSTDILKDQKEPK | |
| | NKTFLRCEAKNYSGRFTCWWLTTISTDLTFSVKSSRGSSDPQGVTCGAATLS | |
| | AERVRGDNKEYEYSVECQEDSACPAAEESLPIEVMVDAVHKLKYENYTSSFF | |
| | IRDIIKPDPPKNLQLKPLKNSRQVEVSWEYPDTWSTPHSYFSLTFCVQVQGK | |
| | SKREKKDRVFTDKTSATVICRKNASISVRAQDRYYSSSWSEWASVPCS | |
| linker between | GGGGSGGGGSGGGGS | 732 |
| IL12p40 and | | |
| IL12p35 | | |
| IL12p35 (without | RNLPVATPDPGMFPCLHHSQNLLRAVSNMLQKARQTLEFYPCTSEEIDHED | 862 |
| leader) | ITKDKTSTVEACLPLELTKNESCLNSRETSFITNGSCLASRKTSFMMALCLSSIY | |
| | EDLKMYQVEFKTMNAKLLMDPKRQIFLDQNMLAVIDELMQALNFNSETV | |
| | PQKSSLEEPDFYKTKIKLCILLHAFRIRAVTIDRVMSYLNAS | |

Fusion polypeptide comprising beta 2 microglobulin leader (B2ML)-HPV16 E7₁₁₋₁₉
peptide-linker-beta 2 microglobulin (B2M)-linker-HLA-A*02:01 Y84A-linker-GPA-snorkel
linker-SMIM1-linker-IL12p40-linker-IL12p35

| | | |
|---|---|---|
| Beta 2 | MSRSVALAVLALLSLSGLEAYMLDLQPETGGGGSGGGGSGGGGSIQRTPKI | 863 |
| microglobulin | QVYSRHPAENGKSNFLNCYVSGFHPSDIEVDLLKNGERIEKVEHSDLSFSKD | |
| leader (B2ML)- | WSFYLLYYTEFTPTEKDEYACRVNHVTLSQPKIVKWDRDMGGGGSGGGGS | |
| HPV16 E7₁₁₋₁₉ | GGGGSGGGGSGSHSMRYFFTSVSRPGRGEPRFIAVGYVDDTQFVRFDSDA | |
| peptide-linker- | ASQRMEPRAPWIEQEGPEYWDGETRKVKAHSQTHRVDLGTLRGAYNQSE | |
| beta 2 | AGSHTVQRMYGCDVGSDWRFLRGYHQYAYDGKDYIALKEDLRSWTAAD | |
| microglobulin (B2M)- | MAAQTTKHKWEAAHVAEQLRAYLEGTCVEWLRRYLENGKETLQRTDAPK | |
| linker-HLA- | THMTHHAVSDHEATLRCWALSFYPAEITLTWQRDGEDQTQDTELVETRPA | |
| A*02:01 Y84A- | GDGTFQKWAAVVVPSGQEQRYTCHVQHEGLPKPLTLRWEPSSQPTIPIGS | |
| linker-GPA- | GSGSGSEDGSGSGSGSLSTTEVAMHTSTSSSVTKSYISSQTNDTHKRDTYAA | |
| snorkel linker- | TPRAHEVSEISVRTVYPPEEETGERVQLAHHFSEPEITLIIFGVMAGVIGTILLI | |
| SMIM1-linker- | SYGIRRLIKKSPSDVKPLPSPDTDVPLSSVEIENPETSDQSGRGASSGSSGSGS | |
| IL12p40-linker- | QKKPRYEIRWKVVVISAILALVVLTVISLIILIMLWGSGMQSPAMQPQESHV | |
| IL12p35 | HYSRWEDGSRDGVSLGAVSSTEEASRCRRISQRLCTGKLGIAMKVLGGVAL | |
| | FWIIFILGYLTGYYVHKCKGGGGSGGGGSGGGGSIWELKKDVYVVELDWYP | |
| | DAPGEMVVLTCDTPEEDGITWTLDQSSEVLGSGKTLTIQVKEFGDAGQYTC | |
| | HKGGEVLSHSLLLLHKKEDGIWSTDILKDQKEPKNKTFLRCEAKNYSGRFTC | |
| | WWLTTISTDLTFSVKSSRGSSDPQGVTCGAATLSAERVRGDNKEYEYSVEC | |
| | QEDSACPAAEESLPIEVMVDAVHKLKYENYTSSFFIRDIIKPDPPKNLQLKPLK | |
| | NSRQVEVSWEYPDTWSTPHSYFSLTFCVQVQGKSKREKKDRVFTDKTSATV | |
| | ICRKNASISVRAQDRYYSSSWSEWASVPCSGGGGSGGGGSGGGGSRNLPV | |
| | ATPDPGMFPCLHHSQNLLRAVSNMLQKARQTLEFYPCTSEEIDHEDITKDK | |
| | TSTVEACLPLELTKNESCLNSRETSFITNGSCLASRKTSFMMALCLSSIYEDLK | |
| | MYQVEFKTMNAKLLMDPKRQIFLDQNMLAVIDELMQALNFNSETVPQKS | |
| | SLEEPDFYKTKIKLCILLHAFRIRAVTIDRVMSYLNAS* | |

Fusion polypeptide comprising beta 2 microglobulin leader (B2ML)-HPV16 E7₁₁₋₁₉
peptide-linker-beta 2 microglobulin (B2M)-linker-HLA-A*02:01 Y84A-linker-GPA-
snorkel linker-linker-IL12p40-linker-IL12p35

| | | |
|---|---|---|
| Beta 2 | MSRSVALAVLALLSLSGLEAYMLDLQPETGGGGSGGGGSGGGGSIQRTPKI | 864 |
| microglobulin | QVYSRHPAENGKSNFLNCYVSGFHPSDIEVDLLKNGERIEKVEHSDLSFSKD | |
| leader (B2ML)- | WSFYLLYYTEFTPTEKDEYACRVNHVTLSQPKIVKWDRDMGGGGSGGGGS | |
| HPV16 E7₁₁₋₁₉ | GGGGSGGGGSGSHSMRYFFTSVSRPGRGEPRFIAVGYVDDTQFVRFDSDA | |
| peptide-linker- | ASQRMEPRAPWIEQEGPEYWDGETRKVKAHSQTHRVDLGTLRGAYNQSE | |
| beta 2 | AGSHTVQRMYGCDVGSDWRFLRGYHQYAYDGKDYIALKEDLRSWTAAD | |
| microglobulin (B2M)- | MAAQTTKHKWEAAHVAEQLRAYLEGTCVEWLRRYLENGKETLQRTDAPK | |
| linker-HLA- | THMTHHAVSDHEATLRCWALSFYPAEITLTWQRDGEDQTQDTELVETRPA | |
| A*02:01 Y84A- | GDGTFQKWAAVVVPSGQEQRYTCHVQHEGLPKPLTLRWEPSSQPTIPIGS | |
| linker-GPA- | GSGSGSEDGSGSGSGSLSTTEVAMHTSTSSSVTKSYISSQTNDTHKRDTYAA | |
| snorkel linker- | TPRAHEVSEISVRTVYPPEEETGERVQLAHHFSEPEITLIIFGVMAGVIGTILLI | |
| linker-IL12p40- | SYGIRRLIKKSPSDVKPLPSPDTDVPLSSVEIENPETSDQSGRGASSGSSGSGS | |
| linker-IL12p35 | QKKPRYEIRWKVVVISAILALVVLTVISLIILIMLWGSGMQSPAGGGGSGGG | |
| | GSGGGGSIWELKKDVYVVELDWYPDAPGEMVVLTCDTPEEDGITWTLDQS | |
| | SEVLGSGKTLTIQVKEFGDAGQYTCHKGGEVLSHSLLLLHKKEDGIWSTDILK | |
| | DQKEPKNKTFLRCEAKNYSGRFTCWWLTTISTDLTFSVKSSRGSSDPQGVTC | |
| | GAATLSAERVRGDNKEYEYSVECQEDSACPAAEESLPIEVMVDAVHKLKYE | |
| | NYTSSFFIRDIIKPDPPKNLQLKPLKNSRQVEVSWEYPDTWSTPHSYFSLTFC | |
| | VQVQGKSKREKKDRVFTDKTSATVICRKNASISVRAQDRYYSSSWSEWASV | |
| | PCSGGGGSGGGGSGGGGSRNLPVATPDPGMFPCLHHSQNLLRAVSNML | |
| | QKARQTLEFYPCTSEEIDHEDITKDKTSTVEACLPLELTKNESCLNSRETSFITN | |
| | GSCLASRKTSFMMALCLSSIYEDLKMYQVEFKTMNAKLLMDPKRQIFLDQN | |
| | MLAVIDELMQALNFNSETVPQKSSLEEPDFYKTKIKLCILLHAFRIRAVTIDRV | |
| | MSYLNAS* | |

Fusion polypeptide comprising beta 2 microglobulin leader (B2ML)-HPV16 E7₁₁₋₁₉ peptide-linker-
beta 2 microglobulin (B2M)-linker-HLA-A*02:01 Y84A-linker-GPA-T2A-GPA signal peptide-IL7-
linker v14-GPA

| | | |
|---|---|---|
| Beta 2 | MSRSVALAVLALLSLSGLEAYMLDLQPETGGGGSGGGGSGGGGSIQRTPKI | 865 |
| microglobulin | QVYSRHPAENGKSNFLNCYVSGFHPSDIEVDLLKNGERIEKVEHSDLSFSKD | |
| leader (B2ML)- | WSFYLLYYTEFTPTEKDEYACRVNHVTLSQPKIVKWDRDMGGGGSGGGGS | |
| HPV16 E7₁₁₋₁₉ | GGGGSGGGGSGSHSMRYFFTSVSRPGRGEPRFIAVGYVDDTQFVRFDSDA | |
| peptide-linker- | ASQRMEPRAPWIEQEGPEYWDGETRKVKAHSQTHRVDLGTLRGAYNQSE | |
| beta 2 | AGSHTVQRMYGCDVGSDWRFLRGYHQYAYDGKDYIALKEDLRSWTAAD | |
| microglobulin (B2M)- | MAAQTTKHKWEAAHVAEQLRAYLEGTCVEWLRRYLENGKETLQRTDAPK | |
| linker-HLA- | THMTHHAVSDHEATLRCWALSFYPAEITLTWQRDGEDQTQDTELVETRPA | |
| A*02:01 Y84A- | GDGTFQKWAAVVVPSGQEQRYTCHVQHEGLPKPLTLRWEPSSQPTIPIGS | |
| linker-GPA-T2A- | GSGSGSEDGSGSGSGSLSTTEVAMHTSTSSSVTKSYISSQTNDTHKRDTYAA | |
| GPA signal peptide- | TPRAHEVSEISVRTVYPPEEETGERVQLAHHFSEPEITLIIFGVMAGVIGTILLI | |
| IL7-linker v14- | SYGIRRLIKKSPSDVKPLPSPDTDVPLSSVEIENPETSDQGSGEGRGSLLTCGD | |
| GPA | VEENPGPMYGKIIFVLLLSEIVSISADCDIEGKDGKQYESVLMVSIDQLLDSM | |
| | KEIGSNCLNNEFNFFKRHICDANKEGMFLFRAARKLRQFLKMNSTGDFDLH | |
| | LLKVSEGTTILLNCTGQVKGRKPAALGEAQPTKSLEENKSLKEQKKLNDLCFL | |
| | KRLLQEIKTCWNKILMGTKEHGGSGGSGGGGGSGGGSGGGSGGGSLSTTE | |
| | VAMHTSTSSSVTKSYISSQTNDTHKRDTYAATPRAHEVSEISVRTVYPPEEET | |
| | GERVQLAHHFSEPEITLIIFGVMAGVIGTILLISYGIRRLIKKSPSDVKPLPSPDT | |
| | DVPLSSVEIENPETSDQ | |
| Beta 2 | MSRSVALAVLALLSLSGLEAYMLDLQPETGGGGSGGGGSGGGGSIQRTPKI | 849 |
| microglobulin | QVYSRHPAENGKSNFLNCYVSGFHPSDIEVDLLKNGERIEKVEHSDLSFSKD | |
| leader (B2ML)- | WSFYLLYYTEFTPTEKDEYACRVNHVTLSQPKIVKWDRDMGGGGSGGGGS | |
| HPV16 E7₁₁₋₁₉ | GGGGSGGGGSGSHSMRYFFTSVSRPGRGEPRFIAVGYVDDTQFVRFDSDA | |
| peptide-linker- | ASQRMEPRAPWIEQEGPEYWDGETRKVKAHSQTHRVDLGTLRGAYNQSE | |
| beta 2 | AGSHTVQRMYGCDVGSDWRFLRGYHQYAYDGKDYIALKEDLRSWTAAD | |
| microglobulin (B2M)- | MAAQTTKHKWEAAHVAEQLRAYLEGTCVEWLRRYLENGKETLQRTDAPK | |
| linker-HLA- | THMTHHAVSDHEATLRCWALSFYPAEITLTWQRDGEDQTQDTELVETRPA | |
| A*02:01 Y84A- | GDGTFQKWAAVVVPSGQEQRYTCHVQHEGLPKPLTLRWEPSSQPTIPIGS | |
| linker-GPA-T2A | GSGSGSEDGSGSGSGSLSTTEVAMHTSTSSSVTKSYISSQTNDTHKRDTYAA | |
| | TPRAHEVSEISVRTVYPPEEETGERVQLAHHFSEPEITLIIFGVMAGVIGTILLI | |
| | SYGIRRLIKKSPSDVKPLPSPDTDVPLSSVEIENPETSDQGSGEGRGSLLTCGD | |
| | VEENPG | |
| GPAsignal peptide- | PMYGKIIFVLLLSEIVSISADCDIEGKDGKQYESVLMVSIDQLLDSMKEIGSNC | 866 |
| IL7-linker v14- | LNNEFNFFKRHICDANKEGMFLFRAARKLRQFLKMNSTGDFDLHLLKVSEG | |
| GPA | TTILLNCTGQVKGRKPAALGEAQPTKSLEENKSLKEQKKLNDLCFLKRLLQEIK | |
| | TCWNKILMGTKEHGGSGGSGGGGGSGGGSGGGSGGGSLSTTEVAMHTS | |
| | TSSSVTKSYISSQTNDTHKRDTYAATPRAHEVSEISVRTVYPPEEETGERVQL | |
| | AHHFSEPEITLIIFGVMAGVIGTILLISYGIRRLIKKSPSDVKPLPSPDTDVPLSS | |
| | VEIENPETSDQ | |
| IL7 (without leader) | DCDIEGKDGKQYESVLMVSIDQLLDSMKEIGSNCLNNEFNFFKRHICDANKE | 867 |
| | GMFLFRAARKLRQFLKMNSTGDFDLHLLKVSEGTTILLNCTGQVKGRKPAA | |
| | LGEAQPTKSLEENKSLKEQKKLNDLCFLKRLLQEIKTCWNKILMGTKEH | |
| Linker v14 | GGSGGSGGGGGSGGGSGGGSGGGS | 737 |

Fusion polypeptide comprising beta 2 microglobulin leader (B2ML)-HPV16 E7₁₁₋₁₉ peptide-
linker-beta 2 microglobulin (B2M)-linker-HLA-A*02:01 Y84A-linker-GPA-T2A-GPA signal
peptide-IL15-linker-IL15Ra-linker v14-GPA

| | | |
|---|---|---|
| Beta 2 | MSRSVALAVLALLSLSGLEAYMLDLQPETGGGGSGGGGSGGGGSIQRTP KI | 868 |
| microglobulin | QVYSRHPAENGKSNFLNCYVSGFHPSDIEVDLLKNGERIEKVEHSDLSFSKD | |
| leader (B2ML)- | WSFYLLYYTEFTPTEKDEYACRVNHVTLSQPKIVKWDRDMGGGGSGGGGS | |
| HPV16 E7₁₁₋₁₉ | GGGGSGGGGSGSHSMRYFFTSVSRPGRGEPRFIAVGYVDDTQFVRFDSDA | |
| peptide-linker- | ASQRMEPRAPWIEQEGPEYWDGETRKVKAHSQTHRVDLGTLRGAYNQSE | |
| beta 2 | AGSHTVQRMYGCDVGSDWRFLRGYHQYAYDGKDYIALKEDLRSWTAAD | |
| microglobulin (B2M)- | MAAQTTKHKWEAAHVAEQLRAYLEGTCVEWLRRYLENGKETLQRTDAPK | |
| linker-HLA- | THMTHHAVSDHEATLRCWALSFYPAEITLTWQRDGEDQTQDTELVETRPA | |
| A*02:01 Y84A- | GDGTFQKWAAVVVPSGQEQRYTCHVQHEGLPKPLTLRWEPSSQPTIPIGS | |
| linker-GPA-T2A- | GSGSGSEDGSGSGSGSLSTTEVAMHTSTSSSVTKSYISSQTNDTHKRDTYAA | |
| GPA signal peptide- | TPRAHEVSEISVRTVYPPEEETGERVQLAHHFSEPEITLIIFGVMAGVIGTILLI | |
| IL15-linker- | SYGIRRLIKKSPSDVKPLPSPDTDVPLSSVEIENPETSDQGSGEGRGSLLTCGD | |
| IL15Ra-linker v14- | VEENPGPMYGKIIFVLLLSEIVSISANWVNVISDLKKIEDLIQSMHIDATLYTES | |
| GPA | DVHPSCKVTAMKCFLLELQVISLESGDASIHDTVENLIILANNSLSSNGNVTE | |
| | SGCKECEELEEKNIKEFLQSFVHIVQMFINTSGGGGSGGGGSGGGGSITCPP | |
| | PMSVEHADIWVKSYSLYSRERYICNSGFKRKAGTSSLTECVLNKATNVAHW | |
| | TTPSLKCIRDPALVHQRPAPPSTVTTAGVTPQPESLSPSGKEPAASSPSSNNT | |
| | AATTAAIVPGSQLMPSKSPSTGTTEISSHESSHGTPSQTTAKNWELTASASH | |
| | QPPGVYPQGHSDTTGGSGGSGGGGGSGGGSGGGSGGGSLSTTEVAMHT | |
| | STSSSVTKSYISSQTNDTHKRDTYAATPRAHEVSEISVRTVYPPEEETGERVQL | |
| | AHHFSEPEITLIIFGVMAGVIGTILLISYGIRRLIKKSPSDVKPLPSPDTDVPLSS | |
| | VEIENPETSDQ | |
| Beta 2 | MSRSVALAVLALLSLSGLEAYMLDLQPETGGGGSGGGGSGGGGSIQRTPKI | 849 |
| microglobulin | QVYSRHPAENGKSNFLNCYVSGFHPSDIEVDLLKNGERIEKVEHSDLSFSKD | |
| leader (B2ML)- | WSFYLLYYTEFTPTEKDEYACRVNHVTLSQPKIVKWDRDMGGGGSGGGGS | |
| HPV16 E7₁₁₋₁₉ | GGGGSGGGGSGSHSMRYFFTSVSRPGRGEPRFIAVGYVDDTQFVRFDSDA | |
| peptide-linker- | ASQRMEPRAPWIEQEGPEYWDGETRKVKAHSQTHRVDLGTLRGAYNQSE | |
| beta 2 | AGSHTVQRMYGCDVGSDWRFLRGYHQYAYDGKDYIALKEDLRSWTAAD | |
| microglobulin (B2M)- | MAAQTTKHKWEAAHVAEQLRAYLEGTCVEWLRRYLENGKETLQRTDAPK | |
| linker-HLA- | THMTHHAVSDHEATLRCWALSFYPAEITLTWQRDGEDQTQDTELVETRPA | |
| A*02:01 Y84A- | GDGTFQKWAAVVVPSGQEQRYTCHVQHEGLPKPLTLRWEPSSQPTIPIGS | |
| linker-GPA-T2A | GSGSGSEDGSGSGSGSLSTTEVAMHTSTSSSVTKSYISSQTNDTHKRDTYAA | |
| | TPRAHEVSEISVRTVYPPEEETGERVQLAHHFSEPEITLIIFGVMAGVIGTILLI | |
| | SYGIRRLIKKSPSDVKPLPSPDTDVPLSSVEIENPETSDQGSGEGRGSLLTCGD | |
| | VEENPG | |
| GPAsignal peptide- | PMYGKIIFVLLLSEIVSISANWVNVISDLKKIEDLIQSMHIDATLYTESDVHPSC | 869 |
| IL15-linker- | KVTAMKCFLLELQVISLESGDASIHDTVENLIILANNSLSSNGNVTESGCKECE | |
| IL15Ra-linker v14- | ELEEKNIKEFLQSFVHIVQMFINTSGGGGSGGGGSGGGGSITCPPPMSVEH | |
| GPA | ADIWVKSYSLYSRERYICNSGFKRKAGTSSLTECVLNKATNVAHWTTPSLKCI | |
| | RDPALVHQRPAPPSTVTTAGVTPQPESLSPSGKEPAASSPSSNNTAATTAA1 | |
| | VPGSQLMPSKSPSTGTTEISSHESSHGTPSQTTAKNWELTASASHQPPGVY | |
| | PQGHSDTTGGSGGSGGGGGSGGGSGGGSGGGSLSTTEVAMHTSTSSSVT | |
| | KSYISSQTNDTHKRDTYAATPRAHEVSEISVRTVYPPEEETGERVQLAHHFSE | |
| | PEITLIIFGVMAGVIGTILLISYGIRRLIKKSPSDVKPLPSPDTDVPLSSVEIENPE | |
| | TSDQ | |
| IL15 (without | NWVNVISDLKKIEDLIQSMHIDATLYTESDVHPSCKVTAMKCFLLELQVISLE | 870 |
| leader) | SGDASIHDTVENLIILANNSLSSNGNVTESGCKECEELEEKNIKEFLQSFVHIV | |
| | QMFINTS | |
| linker between IL15 | GGGGSGGGGSGGGGS | 732 |
| and IL15Ra | | |
| IL15Ra (without | ITCPPPMSVEHADIWVKSYSLYSRERYICNSGFKRKAGTSSLTECVLNKATNV | 871 |
| leader) | AHWTTPSLKCIRDPALVHQRPAPPSTVTTAGVTPQPESLSPSGKEPAASSPS | |
| | SNNTAATTAAIVPGSQLMPSKSPSTGTTEISSHESSHGTPSQTTAKNWELTA | |
| | SASHQPPGVYPQGHSDTT | |

Fusion polypeptide comprising beta 2 microglobulin leader (B2ML)-HPV16 E7₁₁₋₁₉
peptide-linker-beta 2 microglobulin (B2M)-linker-HLA-A*02:01 Y84A-linker-GPA-T2A-
SMIM1-linker-IL12p40-linker-IL12p35

| | | |
|---|---|---|
| Beta 2 | MSRSVALAVLALLSLSGLEAYMLDLQPETGGGGSGGGGSGGGGSIQRTPKI | 872 |
| microglobulin | QVYSRHPAENGKSNFLNCYVSGFHPSDIEVDLLKNGERIEKVEHSDLSFSKD | |
| leader (B2ML)- | WSFYLLYYTEFTPTEKDEYACRVNHVTLSQPKIVKWDRDMGGGGSGGGGS | |
| HPV16 E7₁₁₋₁₉ | GGGGSGGGGSGSHSMRYFFTSVSRPGRGEPRFIAVGYVDDTQFVRFDSDA | |
| peptide-linker- | ASQRMEPRAPWIEQEGPEYWDGETRKVKAHSQTHRVDLGTLRGAYNQSE | |
| beta 2 | AGSHTVQRMYGCDVGSDWRFLRGYHQYAYDGKDYIALKEDLRSWTAAD | |
| microglobulin (B2M)- | MAAQTTKHKWEAAHVAEQLRAYLEGTCVEWLRRYLENGKETLQRTDAPK | |
| linker-HLA- | THMTHHAVSDHEATLRCWALSFYPAEITLTWQRDGEDQTQDTELVETRPA | |
| A*02:01 Y84A- | GDGTFQKWAAVVVPSGQEQRYTCHVQHEGLPKPLTLRWEPSSQPTIPIGS | |
| linker-GPA-T2A- | GSGSGSEDGSGSGSGSLSTTEVAMHTSTSSSVTKSYISSQTNDTHKRDTYAA | |
| SMIM1-linker- | TPRAHEVSEISVRTVYPPEEETGERVQLAHHFSEPEITLIIFGVMAGVIGTILLI | |
| IL12p40-linker- | SYGIRRLIKKSPSDVKPLPSPDTDVPLSSVEIENPETSDQGSGEGRGSLLTCGD | |
| 1L12p35 | VEENPGPMQPQESHVHYSRWEDGSRDGVSLGAVSSTEEASRCRRISQRLC | |
| | TGKLGIAMKVLGGVALFWIIFILGYLTGYYVHKCKGGGGSGGGGSGGGGS1 | |
| | WELKKDVYVVELDWYPDAPGEMVVLTCDTPEEDGITWTLDQSSEVLGSGK | |
| | TLTIQVKEFGDAGQYTCHKGGEVLSHSLLLLHKKEDGIWSTDILKDQKEPKN | |
| | KTFLRCEAKNYSGRFTCWWLTTISTDLTFSVKSSRGSSDPQGVTCGAATLSA | |
| | ERVRGDNKEYEYSVECQEDSACPAAEESLPIEVMVDAVHKLKYENYTSSFFIR | |
| | DIIKPDPPKNLQLKPLKNSRQVEVSWEYPDTWSTPHSYFSLTFCVQVQGKSK | |
| | REKKDRVFTDKTSATVICRKNASISVRAQDRYYSSSWSEWASVPCSGGGGS | |
| | GGGGSGGGGSRNLPVATPDPGMFPCLHHSQNLLRAVSNMLQKARQTLEF | |
| | YPCTSEEIDHEDITKDKTSTVEACLPLELTKNESCLNSRETSFITNGSCLASRKT | |
| | SFMMALCLSSIYEDLKMYQVEFKTMNAKLLMDPKRQIFLDQNMLAVIDEL | |
| | MQALNFNSETVPQKSSLEEPDFYKTKIKLCILLHAFRIRAVTIDRVMSYLNAS | |
| Beta 2 | MSRSVALAVLALLSLSGLEAYMLDLQPETGGGGSGGGGSGGGGSIQRTPKI | 849 |
| microglobulin | QVYSRHPAENGKSNFLNCYVSGFHPSDIEVDLLKNGERIEKVEHSDLSFSKD | |
| leader (B2ML)- | WSFYLLYYTEFTPTEKDEYACRVNHVTLSQPKIVKWDRDMGGGGSGGGGS | |
| HPV16 E7₁₁₋₁₉ | GGGGSGGGGSGSHSMRYFFTSVSRPGRGEPRFIAVGYVDDTQFVRFDSDA | |
| peptide-linker- | ASQRMEPRAPWIEQEGPEYWDGETRKVKAHSQTHRVDLGTLRGAYNQSE | |
| beta 2 | AGSHTVQRMYGCDVGSDWRFLRGYHQYAYDGKDYIALKEDLRSWTAAD | |
| microglobulin (B2M)- | MAAQTTKHKWEAAHVAEQLRAYLEGTCVEWLRRYLENGKETLQRTDAPK | |
| linker-HLA- | THMTHHAVSDHEATLRCWALSFYPAEITLTWQRDGEDQTQDTELVETRPA | |
| A*02:01 Y84A- | GDGTFQKWAAVVVPSGQEQRYTCHVQHEGLPKPLTLRWEPSSQPTIPIGS | |
| linker-GPA-T2A | GSGSGSEDGSGSGSGSLSTTEVAMHTSTSSSVTKSYISSQTNDTHKRDTYAA | |
| | TPRAHEVSEISVRTVYPPEEETGERVQLAHHFSEPEITLIIFGVMAGVIGTILLI | |
| | SYGIRRLIKKSPSDVKPLPSPDTDVPLSSVEIENPETSDQGSGEGRGSLLTCGD | |
| | VEENPG | |
| SMIM1-linker- | PMQPQESHVHYSRWEDGSRDGVSLGAVSSTEEASRCRRISQRLCTGKLGIA | 873 |
| IL12p40-linker- | MKVLGGVALFWIIFILGYLTGYYVHKCKGGGGSGGGGSGGGGSIWELKKD | |
| 1L12p35 | VYVVELDWYPDAPGEMVVLTCDTPEEDGITWTLDQSSEVLGSGKTLTIQVK | |
| | EFGDAGQYTCHKGGEVLSHSLLLLHKKEDGIWSTDILKDQKEPKNKTFLRCE | |
| | AKNYSGRFTCWWLTTISTDLTFSVKSSRGSSDPQGVTCGAATLSAERVRGD | |
| | NKEYEYSVECQEDSACPAAEESLPIEVMVDAVHKLKYENYTSSFFIRDIIKPDP | |
| | PKNLQLKPLKNSRQVEVSWEYPDTWSTPHSYFSLTFCVQVQGKSKREKKDR | |
| | VFTDKTSATVICRKNASISVRAQDRYYSSSWSEWASVPCSGGGGSGGGGS | |
| | GGGGSRNLPVATPDPGMFPCLHHSQNLLRAVSNMLQKARQTLEFYPCTSE | |
| | EIDHEDITKDKTSTVEACLPLELTKNESCLNSRETSFITNGSCLASRKTSFMMA | |
| | LCLSSIYEDLKMYQVEFKTMNAKLLMDPKRQIFLDQNMLAVIDELMQALNF | |
| | NSETVPQKSSLEEPDFYKTKIKLCILLHAFRIRAVTIDRVMSYLNAS | |

In another aspect, the disclosure provides any one of the polypeptide sequences corresponding to a fusion protein listed in Table 9. In one embodiment, the fusion polypeptide comprises Beta 2 microglobulin leader sequence (B2ML)-HPV16 E7₁₁₋₁₉ peptide-Beta 2 microglobulin (B2M)-HLA-A*02:01, set forth in SEQ ID NO: 843. In one embodiment, the fusion polypeptide comprises B2ML-HPV16 E7₁₁₋₁₉ peptide-B2M-HLA-A*02:01 Y84A, set forth in SEQ ID NO: 844. In one embodiments, the fusion polypeptide comprises B2ML-HPV16 E711-19 peptide-B2M-HLA-A*02:01 Y84C, set forth in SEQ ID NO: 845. In one embodiment, the fusion polypeptide comprises B2ML-HPV16 E711-19 peptide-B2M-HLA-A*02:01-Glycophorin A (GPA), set forth in SEQ ID NO: 726. In one embodiment, the fusion polypeptide comprises B2ML-HPV16 E711-19 peptide-B2M-HLA-A*02:01 Y84A-GPA, set forth in SEQ ID NO: 846. In one embodiment, the fusion polypeptide comprises B2ML-HPV16 E7₁₁₋₁₉ peptide-B2M-HLA-A*02:01 Y84C-GPA, set forth in SEQ ID NO: 847. In one embodiment, the fusion polypeptide comprises Beta 2 microglobulin leader (B2ML)-HPV16 E7₁₁₋₁₉ peptide-linker-Beta 2 microglobulin (B2M)-linker-HLA-A*02:01 Y84A-linker-GPA-T2A-GPA signal peptide-N-terminal truncated 4-1BBL-linker v17-GPA, set forth in SEQ ID NO: 848. In one embodiment, the fusion polypeptide comprises Beta 2 Microglobulin leader (B2ML)-HPV16 E7₁₁₋₁₉ peptide-linker-Beta 2 microglobulin (B2M)-linker-HLA-A*02:01 Y84A-linker-GPA-T2A, set forth in SEQ ID NO:849. In one embodiment, the fusion polypeptide comprises SMIM1-linker-IL12p40-linker-IL12p35, set forth in SEQ ID NO: 873. In one embodiment, the fusion polypeptide comprises GPA signal peptide-4-1BBL-linker v17-GPA, set forth in SEQ ID NO: 850. In one embodiment, the fusion polypeptide comprises GPA signal peptide-N-terminal truncated 4-1BBL-linker-GPA-T2A-Beta 2 microglobulin leader (B2ML)-HPV16 E7₁₁₋₁₉ peptide-linker-beta 2 microglobulin-linker-HLA-A*02:01 Y84A-linker-GPA, set forth in SEQ ID NO: 854. In one embodiment, the fusion polypeptide comprises GPA signal peptide-N-terminal truncated 4-1BBL-linker-GPA-T2A, set forth in SEQ ID NO: 855. In one embodiment, the fusion polypeptide comprises T2A-Beta 2 microglobulin leader (B2ML)-HPV16 E7₁₁₋₁₉ peptide-linker-beta 2 microglobulin-linker-HLA-A*02:01 Y84A-linker-GPA, set forth in SEQ ID NO: 856. In one embodiment, the fusion polypeptide comprises Beta 2 microglobulin leader (B2ML)-HPV16 E7₁₁₋₁₉ peptide-linker-beta 2 microglobulin (B2M)-linker-HLA-A*02:01 Y84A-linker-GPA-linker-full length 4-1BBL, set forth in SEQ ID NO: 857. In one embodiment, the fusion polypeptide comprises Beta 2 microglobulin leader (B2ML)-HPV16 E7₁₁₋₁₉ peptide-linker-beta 2 microglobulin (B2M)-linker-HLA-A*02:01 Y84A-linker-GPA-snorkel linker-linker-N-terminal truncated 4-1BBL, set forth in SEQ ID NO: 859. In one embodiment, the fusion polypeptide comprises Beta 2 microglobulin leader (B2ML)-HPV16 E7₁₁₋₁₉ peptide-linker-beta 2 microglobulin (B2M)-linker-HLA-A*02:01 Y84A-linker-GPA-linker-SMIM1-linker-IL12p40-linker-IL12p35, set forth in SEQ ID NO: 860. In one embodiment, the fusion polypeptide comprises Beta 2 microglobulin leader (B2ML)-HPV16 E7₁₁₋₁₉ peptide-linker-beta 2 microglobulin (B2M)-linker-HLA-A*02:01 Y84A-linker-GPA-snorkel linker-SMIM1-linker-IL12p40-linker-IL12p35, set forth in SEQ ID NO: 863. In one embodiment, the fusion polypeptide comprises Beta 2 microglobulin leader (B2ML)-HPV16 E7₁₁₋₁₉ peptide-linker-beta 2 microglobulin (B2M)-linker-HLA-A*02:01 Y84A-linker-GPA-snorkel linker-linker-IL12p40-linker-IL12p35, set forth in SEQ ID NO: 864. In one embodiment, the fusion polypeptide comprises Beta 2 microglobulin leader (B2ML)-HPV16 E7₁₁₋₁₉ peptide-linker-beta 2 microglobulin (B2M)-linker-HLA-A*02:01 Y84A-linker-GPA-T2A-GPA signal peptide-IL7-linker v14-GPA, set forth in SEQ ID NO: 865. In one embodiment, the fusion polypeptide comprises Beta 2 microglobulin leader (B2ML)-HPV16 E7₁₁₋₁₉ peptide-linker-beta 2 microglobulin (B2M)-linker-HLA-A*02:01 Y84A-linker-GPA-T2A, set forth in SEQ ID NO: 849. In one embodiment, the fusion polypeptide comprises GPA signal peptide-IL7-linker v14-GPA, set forth in SEQ ID NO: 866. In one embodiment, the fusion polypeptide comprises Beta 2 microglobulin leader (B2ML)-HPV16 E7₁₁₋₁₉ peptide-linker-beta 2 microglobulin (B2M)-linker-HLA-A*02:01 Y84A-linker-GPA-T2A-GPA signal peptide-IL15-linker-IL15Rα-linker v14-GPA, set forth in SEQ ID NO: 868. In one embodiment, the fusion polypeptide comprises GPA signal peptide-IL15-linker-IL15Rα-linker v14-GPA, set forth in SEQ ID NO: 869. In one embodiment, the fusion polypeptide comprises Beta 2 microglobulin leader (B2ML)-HPV16 E7₁₁₋₁₉ peptide-linker-beta 2 microglobulin (B2M)-linker-HLA-A*02:01 Y84A-linker-GPA-T2A-SMIM1-linker-IL12p40-linker-IL12p35, set forth in SEQ ID NO: 872.

In some embodiments, the disclosure provides a nucleic acid encoding a fusion polypeptide comprising an amino acid sequence set forth in Table 9. In some embodiments, the nucleic acid encodes a fusion polypeptide, wherein the fusion polypeptide has at least 80%, 85%, 90%, 95%, 96%, 97%, 98%, or 99% identity to an amino acid sequence set forth in Table 9. In some embodiments, the nucleic acid encodes a fusion polypeptide, wherein the fusion polypeptide has at least 80%, 85%, 90%, 95%, 96%, 97%, 98%, or 99% identity to SEQ ID NO: 843. In some embodiments, the nucleic acid encodes a fusion polypeptide, wherein the fusion polypeptide has at least 80%, 85%, 90%, 95%, 96%, 97%, 98%, or 99% identity to SEQ ID NO: 844. In some embodiments, the nucleic acid encodes a fusion polypeptide, wherein the fusion polypeptide has at least 80%, 85%, 90%, 95%, 96%, 97%, 98%, or 99% identity to SEQ ID NO: 845. In some embodiments, the nucleic acid encodes a fusion polypeptide, wherein the fusion polypeptide has at least 80%, 85%, 90%, 95%, 96%, 97%, 98%, or 99% identity to SEQ ID NO: 726. In some embodiments, the nucleic acid encodes a fusion polypeptide, wherein the fusion polypeptide has at least 80%, 85%, 90%, 95%, 96%, 97%, 98%, or 99% identity to SEQ ID NO: 846. In some embodiments, the nucleic acid encodes a fusion polypeptide, wherein the fusion polypeptide has at least 80%, 85%, 90%, 95%, 96%, 97%, 98%, or 99% identity to SEQ ID NO: 847. In some embodiments, the nucleic acid encodes a fusion polypeptide, wherein the fusion polypeptide has at least 80%, 85%, 90%, 95%, 96%, 97%, 98%, or 99% identity to SEQ ID NO: 848. In some embodiments, the nucleic acid encodes a fusion polypeptide, wherein the fusion polypeptide has at least 80%, 85%, 90%, 95%, 96%, 97%, 98%, or 99% identity to SEQ ID NO:849. In some embodiments, the nucleic acid encodes a fusion polypeptide, wherein the fusion polypeptide has at least 80%, 85%, 90%, 95%, 96%, 97%, 98%, or 99% identity to SEQ ID NO: 850. In some embodiments, the nucleic acid encodes a fusion polypeptide, wherein the fusion polypeptide has at least 80%, 85%, 90%, 95%, 96%, 97%, 98%, or 99% identity to SEQ ID NO: 854. In some embodiments, the nucleic acid encodes a fusion polypeptide, wherein the fusion polypeptide has at least 80%, 85%, 90%, 95%, 96%, 97%, 98%, or 99% identity to SEQ ID NO: 855. In some embodiments, the nucleic acid encodes a fusion polypeptide, wherein the fusion polypeptide has at least 80%, 85%, 90%, 95%, 96%, 97%, 98%, or 99% identity to SEQ ID NO: 856. In some embodiments, the nucleic acid encodes a fusion polypeptide, wherein the fusion polypeptide has at least 80%, 85%, 90%, 95%, 96%, 97%, 98%, or 99% identity to SEQ ID NO: 857. In some embodiments, the nucleic acid encodes a fusion polypeptide, wherein the fusion polypeptide has at least 80%, 85%, 90%, 95%, 96%, 97%, 98%, or 99% identity to SEQ ID NO: 859. In some embodiments, the nucleic acid encodes a fusion polypeptide, wherein the fusion polypeptide has at least 80%, 85%, 90%, 95%, 96%, 97%, 98%, or 99% identity to SEQ ID NO: 860. In some embodiments, the nucleic acid encodes a fusion polypeptide, wherein the fusion polypeptide has at least 80%, 85%, 90%, 95%, 96%, 97%, 98%, or 99% identity to SEQ ID NO: 863. In some embodiments, the nucleic acid encodes a fusion polypeptide, wherein the fusion polypeptide has at least 80%, 85%, 90%, 95%, 96%, 97%, 98%, or 99% identity to SEQ ID NO: 864. In some embodiments, the nucleic acid encodes a fusion polypeptide, wherein the fusion polypeptide has at least 80%, 85%, 90%, 95%, 96%, 97%, 98%, or 99% identity to SEQ ID NO: 865. In some embodiments, the nucleic acid encodes a fusion polypeptide, wherein the fusion polypeptide has at least 80%, 85%, 90%, 95%, 96%, 97%, 98%, or 99% identity to SEQ ID NO: 849. In some embodiments, the nucleic acid encodes a fusion polypeptide, wherein the fusion polypeptide has at least 80%, 85%, 90%, 95%, 96%, 97%, 98%, or 99% identity to SEQ ID NO: 866. In some embodiments, the nucleic acid encodes a fusion polypeptide, wherein the fusion polypeptide has at least 80%, 85%, 90%, 95%, 96%, 97%, 98%, or 99% identity to SEQ ID NO: 868. In some embodiments, the nucleic acid encodes a fusion polypeptide, wherein the fusion polypeptide has at least 80%, 85%, 90%, 95%, 96%, 97%, 98%, or 99% identity to SEQ ID NO: 869. In some embodiments, the nucleic acid encodes a fusion polypeptide, wherein the fusion polypeptide has at least 80%, 85%, 90%, 95%, 96%, 97%, 98%, or 99% identity to SEQ ID NO: 872.

In some embodiments, the polypeptide comprising 4-1BBL is an N-terminal truncated 4-1BBL (SEQ ID NO: 851). In some embodiments, the polypeptide comprising 4-1BBL is full length 4-1BBL.

Also provided by the present disclosure is an aAPC comprising a first exogenous polypeptide and a second exogenous polypeptide, wherein the first exogenous polypeptide comprises a fusion protein comprising an exogenous antigenic peptide, an exogenous antigen presenting polypeptide and a membrane anchor polypeptide, wherein the second exogenous polypeptide comprises one or more polypeptides selected from the group consisting of: an exogenous co-stimulatory polypeptide, an exogenous co-inhibitory polypeptide, an exogenous Treg expansion polypeptide, and an exogenous cytokine polypeptide, and wherein the aAPC is produced by a process comprising introducing an exogenous nucleic acid encoding the first exogenous polypeptide into a nucleated cell (e.g., nucleated erythroid precursor cell); introducing an exogenous nucleic acid encoding the second exogenous polypeptide into the nucleated cell (e.g., nucleated erythroid precursor cell); and culturing the nucleated cell (e.g., nucleated erythroid precursor cell) under conditions suitable for enucleation and for production of both the first exogenous polypeptide and the second exogenous polypeptide. In some embodiments, the exogenous antigenic polypeptide is selected from an antigenic polypeptide disclosed in Table 1 or Tables 14-24. In some embodiments, the first exogenous polypeptide comprises a fusion protein comprising an exogenous antigenic peptide fused to an exogenous antigen presenting polypeptide fused to a membrane anchor polypeptide. In some embodiments, the exogenous antigenic polypeptide is selected from the group consisting of: melanoma antigen genes-A (MAGE-A) antigens, neutrophil granule protease antigens, NY-ESO-1/LAGE-2 antigens, telomerase antigens, myelin oligodendrocyte glycoprotein (MOG) antigens, glycoprotein 100 (gp100) antigens, epstein barr virus (EBV) antigens, human papilloma virus (HPV) antigens, and hepatitis B virus (HBV) antigens. In some embodiments, the exogenous antigenic polypeptide further comprises a leader sequence. In some embodiments, the leader sequence is a beta 2 microglobulin (B2M) leader sequence or a GPA signal peptide. In some embodiments, the membrane anchor is glycophorin A (GPA), or a fragment thereof, or small integral membrane protein 1 (SMIM1) In some embodiments, the exogenous antigen-presenting polypeptide is an MHC class I polypeptide, an MHC class I single chain fusion, an MHC class II polypeptide, or an MHC class II single chain fusion. In some embodiments, the MHC class I polypeptide is selected from the group consisting of: HLA-A, HLA-B, and HLA-C. In some embodiments, the MHC class II polypeptide is selected from the group consisting of: HLA-DPα, HLA-DPβ, HLA-DM, HLA-DOA, HLA-DOB, HLA-DQα, HLA-DQβ, HLA-DRα, and HLA-DRβ. In some embodiments, the MHC class I single chain fusion comprises an an α-chain, and a β2m chain, and optionally an anchor polypeptide. In some embodiments, the exogenous antigenic polypeptide is connected to the MHC I single chain fusion via a linker. In some embodiments, the linker is a cleavable linker. In some embodiments, the MHC class II single chain fusion comprises an anchor, an α-chain, and optionally a β chain. In some embodiments, the exogenous antigenic polypeptide is connected to the MHC II single chain fusion via a linker. In some embodiments, the linker is a cleavable linker. In some embodiments, the exogenous cytokine polypeptide is selected from the group consisting of: IL2, IL15, IL-15Rα fused to IL-15, IL7, IL12, IL18, IL21, IL4, IL6, IL23, IL27, IL17, IL10, TGF-beta, IFN-gamma, IL-1 beta, GM-CSF, and IL-25. In some embodiments, the exogenous costimulatory polypeptide is selected from the group consisting of 4-1BBL, LIGHT, anti CD28, CD80, CD86, CD70, OX40L, GITRL, TIM4, SLAM, CD48, CD58, CD83, CD155, CD112, IL-15Rα fused to IL-15, IL-21, ICAM-1, a ligand for LFA-1, anti CD3, and a combination thereof. In some embodiments, the exogenous co-inhibitory polypeptide is selected from the group consisting of: IL-35, IL-10, VSIG-3 and a LAG3 agonist. In some embodiments, the exogenous Treg expansion polypeptide is selected from the group consisting of: CD25-specific IL-2, TNFR2-specific TNFα, antiDR3 agonist (VEGI/TL1A specific), 4-1BBL, TGFβ, and a combination thereof. In some embodiments, the aAPC further comprises an exogenous polypeptide comprising an adhesion molecule. In some embodiments, the adhesion molecule is selected from the group consisting of: ICAM4/LW, CD36, CD58/LFA3, CD47, VLA4, BCAM/Lu, CD44, CD99/MIC2, ICAM1, and CD147. In some embodiments, the aAPC of claim 90, wherein the exogenous nucleic acid comprises DNA or RNA. In some embodiments, the introducing step comprises viral transduction or electroporation. In some embodiments, the introducing step comprises utilizing one or more of: liposome mediated transfer, adenovirus, adeno-associated virus, herpes virus, a retroviral based vector, lipofection, and a lentiviral vector. In some embodiments, the introducing step comprises introducing the first exogenous nucleic acid encoding the first exogenous polypeptide and the second exogenous nucleic acid encoding the second exogenous polypeptide by transduction with a lentiviral vector, wherein the first exogenous nucleic acid and the second exogenous nucleic acid are contained in the same lentiviral vector. In some embodiments, the introducing step comprises introducing the first exogenous nucleic acid encoding the first exogenous polypeptide by transduction with a first lentiviral vector, and introducing the second exogenous nucleic acid encoding the second exogenous polypeptide by transduction with a second lentiviral vector. In some embodiments, the first and/or second exogenous nucleic acid comprises a promoter selected from the group consisting of: beta-globin promoter, murine stem cell virus (MSCV) promoter, Gibbon ape leukemia virus (GALV) promoter, human elongation factor 1 alpha (EF1alpha) promoter, CAG CMV immediate early enhancer and the chicken beta-actin (CAG) promoter, and human phosphoglycerate kinase 1 (PGK) promoter.

### Immunological Synapse

As described herein, the engineered erythroid cells (i.e. the aAPCs) of the present disclosure provide numerous advantages over the use of spherical nanoparticles, such as rigid, bead-based aAPCs. Molecular mobility (e.g. movement of ligands in the cell membrane) and molecular clustering are important features of immunological synapse formation. The membrane of an aAPC described herein is much more dynamic and fluid than the outer surface of a nanoparticle, and thus allows a much more efficient molecular reorganization and MHC clustering during the formation of an immunological synapse, or in mediating trogocytosis. Further, in contrast to the small size of the nanoparticles, the aAPC's of the invention offer a greater surface area for the formation of functional micron-scaled clusters in an immunological synapse. In some embodiments, the aAPCs as described herein are engineered to form an immunological synapse, wherein the immunological synapse facilitates T cell activation.

An immunological synapse (or immune synapse, or IS) is the interface between an antigen-presenting cell and a lymphocyte such as a T/B cell or an NK cell. An immunological synapse can consist of molecules involved in T cell activation, which compose typical patterns, called activation clusters. According to the most well studied model, the immune synapse is also known as the supramolecular activation cluster (SMAC) (Monks et al., Nature 1998, 395 (6697): 82-86; incorporated in their entirety herein by reference), which is composed of concentric rings (central, peripheral or distal regions) each containing segregated clusters of proteins. Molecules in the immunological synapse include antigen presenting molecules (e.g. an MHC Class I or MHC Class II molecule), adhesion molecules, co-stimulatory molecules, and co-inhibitory molecules.

The immunological synapse is a dynamic structure formed after T cell receptors cluster together in microclusters that eventually move towards the immunological synapse center. The spatial and temporal changes of these molecules at the interface of T lymphocyte and APC regulate the structure of the immune synapse and T lymphocyte immune response. In general, efficient CD4+ and CD8+ T cell activation is associated with the formation of a functional immunological synapse (Y. Kaizuka, et al. Proc. Natl. Acad. Sci. U.S.A., 104 (2007), pp. 20296-20301, incorporated by reference in its entirety herein).

In some embodiments, the disclosure features an aAPC that can form an immunological synapse between the aAPC and an immune cell such as a T cell, B cell or an NK cell. In some embodiments, the aAPC of the invention has the ability to assemble more than one MHC molecule in the immunological synapse.

The initial interaction at the immunological synapse occurs between the lymphocyte function-associated antigen-1 (LFA-1) present in the peripheral-SMAC of a T-cell, and integrin adhesion molecules (such as ICAM-1 or ICAM-2) on an APC. When bound to an APC, the T-cell can then extend pseudopodia and scan the surface of target cell to find a specific peptide-MHC complex. The process of formation begins when the T-cell receptor (TCR) binds to the peptide-MHC complex on the antigen-presenting cell and initiates signaling activation through formation of microclusters/lipid rafts (Varma et al., Immunity. 2006 July; 25(1):117-27; incorporated in their entirety herein by reference).

It is a surprising discovery of the present disclosure that the engineered erythroid cells or enucleated cells (i.e. the aAPCs) of the invention are capable of initiating and forming an active immunological synapse despite the absence of endogenous ICAM1 on their surface. Without wishing to be bound by any particular theory, it is believed that other integrins such as JAM1 and/or ICAM-4, which are naturally present on the surface of erythroid cells, are capable of replacing the role of ICAM-1 in the formation of a functional immunological synapse.

Accordingly, in some embodiments, the aAPCs of the present disclosure comprise one or more exogenous cell adhesion polypeptides to mediate or facilitate the formation of the immunological synapse. In some embodiments, the one or more cell adhesion molecule is selected from the group consisting of ICAM4/LW, CD36, CD58/LFA3, CD47, VLA4, BCAM/Lu, CD44, CD99/MIC2, ICAM1, JAM1 and CD147, or any combination thereof.

It is an advantage of the present invention that the engineered erythroid cells (i.e. the aAPCs) described herein have a fluid cell membrane that provides dynamic molecular movement and thus allows efficient molecular reorganization and MHC clustering, which is required for T cell stimulation. Signaling is initiated and sustained in TCR microclusters that are formed continuously in the periphery of the immunological synapse and transported to the center to form the central SMAC. During the formation of the central SMAC the microclusters can move independently of each other, and can fuse to form larger clusters with continuous movements. A threshold MHCI cluster density is required to sustain active immune signaling (Anikeeva et al., PLoS One. 2012; 7(8):e41466; Bullock et al., J Immunol. 2000 Mar. 1; 164(5):2354-61; Bullock et al., J Immunol. 2003 Feb. 15; 170(4):1822-9; Jiang et al., Immunity. 2011 Jan. 28; 34(1):13-23; each of which is incorporated in its entirety herein by reference). Accordingly, in some embodiments, an aAPC provided herein can mediate the clustering of MHC molecules at a density that is effective to form a functional immunological synapse and to activate immune signaling.

Another consequence of the molecular reorganization in immune synapse formation is the intercellular transfer of APC membrane proteins to the T cell. T cells acquire MHC class I and class II glycoproteins from APCs, together with co-stimulatory molecules and membrane patches, by a mechanism referred to as trogocytosis. As described herein, the membrane of an aAPC provided herein allows efficient molecular reorganization and MHC clustering due to its fluidity. In some embodiments, an aAPC of the invention allows or mediates the molecular reorganization in immune synapse formation such that trogocytosis occurs.

The sizes of the immunological synapse can be determined by numerous methods known in the art, including microscopy, such as total internal reflection fluorescence microscopy (TIRFM) (Varma et al., 2006). Studies have shown that the immunological synapse is composed of micron-scale SMACs (Varma et al., 2006; Dustin et al., Science. 2002, 298(5594):785-9; incorporated in their entirety herein by reference). In some embodiments, an aAPC of the invention can form an immunological synapse of an average diameter between about 0.5 μm and 5.0 μm. In some embodiments, an aAPC of the invention can form an immunological synapse of an average diameter of at least about 0.5 μm. In some embodiments, an aAPC of the invention can form a functional immunological synapse of an average diameter between about 0.5 μm and 4.5 μm, between about 0.5 μm and 4.0 μm, between about 0.5 μm and 3.5 μm, between about 0.5 μm and 3.0 μm, between about 0.5 μm and 2.5 μm, between about 0.5 μm and 2.0 μm, between about 0.5 μm and 1.5 μm, between about 0.5 μm and 1.0 μm, between about 1.0 μm and 5.0 μm, between about 1.0 μm and 4.5 μm, between about 1.0 μm and 4.0 μm, between about 1.0 μm and 3.5 μm, between about 1.0 μm and 3.0 μm, between about 1.0 μm and 2.5 μm, between about 1.0 μm and 2.0 μm, between about 1.0 μm and 1.5 μm, between about 1.5 μm and 5.0 μm, between about 1.5 μm and 4.5 μm, between about 1.5 μm and 4.0 μm, between about 1.5 μm and 3.5 μm, between about 1.5 μm and 3.0 μm, between about 1.5 μm and 2.5 μm, between about 1.5 μm and 2.0 μm, between about 2.0 μm and 5.0 μm, between about 2.0 μm and 4.5 μm, between about 2.0 μm and 4.0 μm, between about 2.0 μm and 3.5 μm, between about 2.0 μm and 3.0 μm, between about 2.0 μm and 2.5 μm, between about 2.0 μm and 5.0 μm, between about 2.5 μm and 4.5 μm, between about 2.5 μm and 4.0 μm, between about 2.5 μm and 3.5 μm, between about 2.5 μm and 3.0 μm, between about 3.0 μm and 5.0 μm, between about 3.0 μm and 4.5 μm, between about 3.0 μm and 4.0 μm, between about 3.0 μm and 3.5 μm, between about 3.5 μm and 5.0 μm, between about 3.5 μm and 4.5 μm, between about 3.5 μm and 4.0 μm, between about 4.0 μm and 5.0 μm, between about 4.0 μm and 4.5 μm, between about 4.5 μm and 5.0 μm. In some embodiments, the aAPC of the invention can form a functional immunological synapse of an average diameter of at least 0.5 μm, 0.6 μm, 0.7 μm, 0.8 μm, 0.9 μm, 1.0 μm, 1.5 μm, 2.0 μm, 2.5 μm, 3 μm, 3.5 μm, 4.0 μm or 5 μm.

As described herein, an advantage of the aAPCs of the present disclosure is the fluidity of the aAPC cell membrane that allows efficient molecular reorganization. Specific signaling pathways lead to polarization of the T-cell by orienting its centrosome toward the site of the immunological synapse. The accumulation and polarization of actin is triggered by TCR/CD3 interactions with integrins and small GTPases. These interactions promote actin polymerization, and as actin is accumulated and reorganized, it promotes clustering of the TCRs and integrins. These highly dynamic contacts are characterized by continuous cytoskeletal remodeling events, which not only structure the interface but also exert a considerable amount of mechanical forces, which influence information transfer both into and out of the immune cell (Basu et al., Trends Cell Biol. 2017 April; 27(4): 241-254; Hivroz et al., Front Immunol. 2016; 7: 46; incorporated in their entirety herein by reference). The adhesive forces of tensile strengths between the TCRs and integrins at the site of immunological synapse can be determined by, e.g., atomic force microscopy, biomembrane force probe (BFP) technique, traction force microscopy etc. (Hivroz et al., Front Immunol. 2016; 7: 46; incorporated in its entirety herein by reference).

In some embodiments, tensile strength is a measure of the adhesive forces between the T cell receptor and the molecules of the immunological synapse, e.g., peptide-MHC complex, formed by the aAPC. In some embodiments, an aAPC is capable of forming an immunological synapse with a tensile strength sufficient to activate an immune cell. In some embodiments, an aAPC of the present disclosure can form a synapse with a tensile strength of between about 1 pN and 30,000 pN. In some embodiments, an aAPC of the present disclosure can form a synapse with a tensile strength of between about 1 pN and 20,000 pN, between about 1 pN and 10,000 pN, between about 1 pN and 9,000 pN, between about 1 pN and 8,000 pN, between about 1 pN and 7,000 pN, between about 1 pN and 6,000 pN, between about 1 pN and 5,000 pN, between about 1 pN and 4,000 pN, between about 1 pN and 3,000 pN, between about 1 pN and 2,000 pN, between about 1 pN and 1,000 pN, between about 1,000 pN and 30,000 pN, between about 1,000 pN and 20,000 pN, between about 1,000 pN and 10,000 pN, between about 1,000 pN and 9,000 pN, between about 1,000 pN and 8,000 pN, between about 1,000 pN and 7,000 pN, between about 1,000 pN and 6,000 pN, between about 1,000 pN and 5,000 pN, between about 1,000 pN and 4,000 pN, between about 1,000 pN and 3,000 pN, between about 1,000 pN and 2,000 pN. In some embodiments, the optimum mechanical force between the peptide-MHC complex and the TCR at the immunological synapse is at least 1 pN, 1.5 pN, 2.0 pN, 3.0 pN, 4.0 pN, 5.0 pN, 6.0 pN, 7.0 pN, 8.0 pN, 9.0 pN, 10 pN, 20 pN, 30 pN, 40 pN, 50 pN, 60 pN, 70 pN, 80 pN, 90 pN, 100 pN, 500 pN, 1,000 pN, 2,000 pN, 3,000 pN, 4,000 pN, 5,000 pN, 6,000 pN, 7,000 pN, 8,000 pN, 9,000 pN, 10,000 pN, 11,000 pN, 12,000 pN, 13,000 pN, 14,000 pN, 15,000 pN, or 20,000 pN. In some embodiments, an aAPC as described herein can trigger mechanical forces between the peptide-MHC complex and the TCR at the immunological synapse, to activate an immune cell.

### Treg Costimulatory and Coinhibitory Polypeptides

Regulatory T cells (“Treg”) are a specialized subpopulation of T cells which suppresses activation of the immune system and thereby maintains tolerance to self-antigens. Treg cells constitute 5-10% of CD4⁺ T cells in humans and rodents. Treg cells constitute 5-10% of CD4⁺ T cells in humans and rodents, and constitutively express CD4 and CD25, as well as the transcription factor FoxP3 (CD4+CD25+FoxP3+), which is involved in their development and function. IL-2 also appears to play an important role in Treg cell development and homeostasis because animals deficient for IL-2 or components of its receptor develop T cell hyperproliferation and autoimmune diseases that can be corrected by adoptive transfer of Treg cells from naive animals. Similarly, a lack of signaling through CD28/CD80 interaction is associated with reduced number and functionality of Treg cells, suggesting that this receptor/ligand system plays an important role in the development and function of Treg cells.

In certain embodiments, the present disclosure features Treg costimulatory polypeptides that are exogenous polypeptides that expand regulatory T-cells (Tregs) cells. In some embodiments, the Treg costimulatory polypeptides expand Treg cells by stimulating at least one of three signals involved in Treg cell development. Signal 1 involves TCR, and can be stimulated with antibodies, such as anti-CD3 antibodies, or with antigens that signals through TCR. Signal 2 can be mediated by several different molecules, including immune co-stimulatory molecules such as CD80 and 4-1BBL. Signal 3 is transduced via cytokines, such as IL-2, or TGFβ. In some embodiments, the Treg costimulatory polypeptides stimulate one of these signals. In another embodiment, the Treg costimulatory polypeptides stimulate two of these signals. In yet another embodiment, the Treg costimulatory polypeptides stimulate three of these signals.

Signal 1

Antigens useful as Treg costimulatory polypeptides for stimulating Signal 1 include antigens associated with a target disease or condition. For example, autoantigens and insulin (particularly suitable for treating type 1 diabetes), collagen (particularly suitable for treating rheumatoid arthritis), myelin basic protein (particularly suitable for treating multiple sclerosis) and MHC (for treating and preventing foreign graft rejection). The antigens may be administered as part of a conjugate. Optionally, the antigen is provided as part of an MHC/antigen complex. In this embodiment, the MHC and antigen can independently be foreign or syngeneic. For example donor MHC and an allogenic or syngeneic antigen can be used.

Signal 2

Exemplary Treg costimulatory polypeptides for stimulating Signal 2 include members of the B7 and TNF families, for example B7 and CD28 family members, shown below in Table 10, and TNF family members shown in Table 11.

TABLE 10
Treg Costimulatory Polypeptides: B7 and CD28 Family Members

| LIGAND | RECEPTOR |
|---|---|
| B7.1 (CD80) | CD28, CTLA-4 (CD 152) |
| B7.2 (CD86) | CD28, CTLA-4 |
| ICOSL (B7h, B7-H2, B7RP-1, | ICOS (AILIM) |
| GL5O, LICOS) | |
| PD-L1 (B7-H1) | PD-1 |
| PD-L2 (B7-DC) | PD-1 |
| B7-H3 | Unknown |
| B7-H4 (B7x; B7S1) | Unknown (BTLA?) |
| Unknown (HVEM*) | BTLA |
| ICOSL (B7h, B7-H2, B7RP-1, | ICOS (AILIM) |

TABLE 11
Treg Costimulatory Polypeptides: TNF Family Members

| LIGAND | RECEPTOR |
|---|---|
| OX40L | OX40 (CD134) |
| 4-1BBL | 4-1BB (CD137) |
| CD40L (CD154) | CD40 |
| CD27L (CD70) | CD27 |
| CD30L | CD30 |
| LIGHT | HVEM, LTβR, DcR3 |
| GITRL | GITR |
| BAFF (BLyS) ** | BAFF-R, TACI, BCMA |
| APRIL ** | TACI, BCMA |
| VEGI/TL1A | DR3 |
| TNF alpha (mutants) | TNFR2 |

Signal 3

Exemplary Treg costimulatory polypeptides for stimulating Signal 3 include cytokines and growth factors that stimulate Signal 3, such as IL-2, IL-4, and TGF-β (including TGF-β1, TGF-β2 and TGF-β3). IL-2 and IL-4 moieties useful in immunotherapeutic methods are known in the art. See, e.g., Earle et al., 2005, supra; Thorton et al., 2004, J. Immunol. 172: 6519-23; Thorton et al., 2004, Eur. J. Immunol. 34: 366-76. In accordance with one embodiment, the mature portion of the cytokine is used.

In some embodiments, the Treg costimulatory polypeptide is CD25-specific IL-2. In some embodiments, the Treg costimulatory polypeptide is TNFR2-specific TNF. In some embodiments, the Treg costimulatory polypeptide is an anti-DR3 agonist (VEGI/TL1A specific). In some embodiments, the Treg costimulatory peptide is 4-1BBL. In some embodiments, the Treg costimulatory peptide is TGFbeta.

In other embodiments, the present disclosure features Treg co-inhibitory polypeptides that are exogenous polypeptides that inhibit Treg cells. In certain embodiments, Treg inhibition is useful in the treatment of cancer, for example, by targeting chemokines that are involved in Treg trafficking. Other Treg inhibitors can target any of the receptors listed in Tables 10 or 11, for example, anti-OX40, anti-GITR or anti-CTLA4, or TLR ligands.

In some embodiments, the Treg costimulatory polypeptides, or an active fragment thereof, can be linked or expressed as a fusion protein with a binding pair member for use in accordance with the present invention. An exemplary binding pair is biotin and streptavidin (SA) or avidin.

In some embodiments, the Treg costimulatory polypeptides, or an active fragment thereof, is part of a fusion protein, comprising a Treg costimulatory polypeptide and a binding pair member, such as CSA. Fusion proteins can be made by any of a number of different methods known in the art. For example, one or more of the component polypeptides of the fusion proteins can be chemically synthesized or can be generated using well known recombinant nucleic acid technology. (As used herein, “nucleic acid” refers to RNA or DNA.) Nucleic acid sequences useful in the present invention can be obtained using, for example, the polymerase chain reaction (PCR). Various PCR methods are described, for example, in PCR Primer: A Laboratory Manual, Dieffenbach 7 Dveksler, Eds., Cold Spring Harbor Laboratory Press, 1995. Fusions are discussed in more detail herein below.

The conjugate may include a linker such as a peptide linker between the binding pair member and the costimulatory moiety. The linker length and composition may be chosen to enhance the activity of either functional end of the moiety. The linker may be greater than 20 amino acids long. In some embodiments, the linker is generally from about 3 to about 30 amino acids long, for example about 5 to about 20 amino acids long, about 5 to about 15 amino acids long, about a to about 10 amino acids long. However, longer or shorter linkers may be used or the linker may be dispensed with entirely. Flexible linkers (e.g. (Gly4Ser)3 (SEQ ID NO: 1)) such as have been used to connect heavy and light chains of a single chain antibody may be used in this regard. See, e.g., Huston et al., 1988, Proc. Nat. Acad. Sci. USA, 85: 5879-5883; U.S. Pat. Nos. 5,091,513, 5,132,405, 4,956,778; 5,258,498, and 5,482,858, the entireties of each of which is incorporated by reference herein. Other linkers are FENDAQAPKS (SEQ ID NO: 717) or LQNDAQAPKS (SEQ ID NO: 718). One or more domains of an immunoglobulin Fc region (e.g CH1, CH2 and/or CH3) also may be used as a linker.

In certain embodiments, the polypeptide is an exogenous Treg costimulatory polypeptide as described herein. An exemplary Treg costimulatory polypeptide includes:

a) a naturally occurring form of the human polypeptide;

b) the human polypeptide having a sequence appearing in a database, e.g., GenBank database, on Dec. 22, 2017;

c) a human polypeptide having a sequence that differs by no more than 1, 2, 3, 4, 5 or 10 amino acid residues from a sequence of a) or b);

d) a human polypeptide having a sequence that differs at no more than 1, 2, 3, 4, 5 or 10% its amino acids residues from a sequence of a) or b);

e) a human polypeptide having a sequence that does not differ substantially from a sequence of a) or b); or

f) a human polypeptide having a sequence of c), d), or e) that does not differ substantially in a biological activity, e.g., an enzymatic activity (e.g., specificity or turnover) or binding activity (e.g., binding specificity or affinity) from a human polypeptide having the sequence of a) or b). Candidate peptides under f) can be made and screened for similar activity as described herein and would be equivalent hereunder if expressed in engineered erythroid cells as described herein).

In embodiments, an exogenous Treg costimluatory polypeptide comprises a human polypeptide or fragment thereof, e.g., all or a fragment of a human polypeptide of a), b), c), d), e), or f) of the preceding paragraph. In an embodiment, the exogenous Treg costimulatory polypeptide comprises a fusion polypeptide comprising all or a fragment of a human polypeptide of a), b), c), d), e), or f) of the preceding paragraph and additional amino acid sequence. In an embodiment the additional amino acid sequence comprises all or a fragment of human polypeptide of a), b), c), d), e), or f) of the preceding paragraph for a different human Treg costimulatory polypeptide.

In some embodiments, the aAPC presents, e.g. comprises on the cell surface, at least two, at least 3, at least 4, or at least 5 exogenous Treg costimulatory polypeptides.

In some embodiments, the one or more Treg co-stimulatory or co-inhibitory polypeptides include or are fused to a membrane anchor. In some embodiments, the membrane anchor is selected from a sequence set forth in Table 3. In some embodiments, the one or more Treg co-stimluatory or co-inhibitory polypeptides include or are fused to a leader sequence. In some embodiments, the leader sequence is selected from a sequence set forth in Table 2.

### Exogenous Metabolite-Altering Polypeptides

In some embodiments of the present invention, an exogenous metabolite-altering polypeptide refers to any polypeptide involved in the catabolism or anabolism of a metabolite in a cell, wherein the metabolite-altering polypeptide can affect the metabolism of a T cell. Exemplary metabolite-depleting polypeptides as described herein alter the level of metabolites in the cell's local environment. For example, in some embodiments, a metabolite-depleting polypeptide promotes the oxidative catabolism of tryptophan.

Exemplary metabolite-altering polypeptides include CD39, CD73, arginase (Arg1) that can be used for the depletion of arginine, indoleamine 2,3-dioxygenase (IDO) which can be used for the depletion of tryptophan; tryptophan 2,3-dioxygenase (TDO-2) inhibitors that can be used for the depletion of tryptophan; tryptophan 5-hydroxylase (TPH) inhibitors that reduce 5-HT synthesis and can be used for the depletion of tryptophan; cyclooxyegnase-2 (COX-2) and prostaglandin (PGE) synthase (PGES), which can be used for the generation of prostaglandin E2 (PGE2); and inducible nitric oxide synthase (iNOS), that can be used for the generation of NO.

In certain embodiments, the polypeptide is an exogenous metabolite-altering polypeptide as described herein. An exemplary metabolite-altering polypeptide includes:

a) a naturally occurring form of the human polypeptide;

b) the human polypeptide having a sequence appearing in a database, e.g., GenBank database, on Dec. 22, 2017;

c) a human polypeptide having a sequence that differs by no more than 1, 2, 3, 4, 5 or 10 amino acid residues from a sequence of a) or b);

d) a human polypeptide having a sequence that differs at no more than 1, 2, 3, 4, 5 or 10% its amino acids residues from a sequence of a) or b);

e) a human polypeptide having a sequence that does not differ substantially from a sequence of a) or b); or

f) a human polypeptide having a sequence of c), d), or e) that does not differ substantially in a biological activity, e.g., an enzymatic activity (e.g., specificity or turnover) or binding activity (e.g., binding specificity or affinity) from a human polypeptide having the sequence of a) or b). Candidate peptides under f) can be made and screened for similar activity as described herein and would be equivalent hereunder if expressed in engineered erythroid cells as described herein).

In embodiments, an exogenous metabolite-altering polypeptide comprises a human polypeptide or fragment thereof, e.g., all or a fragment of a human polypeptide of a), b), c), d), e), or f) of the preceding paragraph. In an embodiment, the exogenous metabolite-altering polypeptide comprises a fusion polypeptide comprising all or a fragment of a human polypeptide of a), b), c), d), e), or f) of the preceding paragraph and additional amino acid sequence. In an embodiment the additional amino acid sequence comprises all or a fragment of human polypeptide of a), b), c), d), e), or f) of the preceding paragraph for a different human metabolite-altering polypeptide.

In some embodiments, the one or more exogenous metabolite-altering polypeptides include or are fused to a membrane anchor. In some embodiments, the membrane anchor is selected from a sequence set forth in Table 3. In some embodiments, the one or more exogenous metabolite-altering polypeptides include or are fused to a leader sequence. In some embodiments, the leader sequence is selected from a sequence set forth in Table 2.

### Cytokines/Chemokines

Additionally, the disclosure encompasses an aAPC transduced with a nucleic acid encoding at least one cytokine, at least one chemokine, or both. Thus, the disclosure encompasses a cytokine, including a full-length, fragment, homologue, variant or mutant of the cytokine. A cytokine includes a protein that is capable of affecting the biological function of another cell. A biological function affected by a cytokine can include, but is not limited to, cell growth, cell differentiation or cell death. Preferably, a cytokine of the present disclosure is capable of binding to a specific receptor on the surface of a cell, thereby affecting the biological function of a cell.

A preferred cytokine includes, among others, a hematopoietic growth factor, an interleukin, an interferon, an immunoglobulin superfamily molecule, a tumor necrosis factor family molecule and/or a chemokine. A more preferred cytokine of the disclosure includes a granulocyte macrophage colony stimulating factor (GM-CSF), tumor necrosis factor alpha (TNFα), tumor necrosis factor beta (TNFβ), macrophage colony stimulating factor (M-CSF), interleukin-1 (IL-1), interleukin-2 (IL-2), interleukin-4 (IL-4), interleukin-5 (IL-5), interleukin-6 (IL-6), interleukin-7 (IL-7), interleukin-10 (IL-10), interleukin-12 (IL-12), interleukin-15 (IL-15), interleukin-21 (IL-21), interleukin-35 (IL-35), interferon alpha (IFN-α), interferon beta (IFN-β), interferon gamma (IFN-γ), and IGIF, among many others.

A chemokine, including a homologue, variant, mutant or fragment thereof, encompasses an alpha-chemokine or a beta-chemokine, including, but not limited to, a C5a, interleukin-8 (IL-8), monocyte chemotactic protein 1alpha (MIP1a), monocyte chemotactic protein 1 beta (MIP1β), monocyte chemoattractant protein 1 (MCP-1), monocyte chemoattractant protein 3 (MCP-3), platelet activating factor (PAFR), N-formyl-methionyl-leucyl-[³H]phenylalanine (FMLPR), leukotriene B₄ (LTB₄R), gastrin releasing peptide (GRP), RANTES, eotaxin, lymphotactin, IP10, 1-309, ENA78, GCP-2, NAP-2 and/or MGSA/gro. One skilled in the art would appreciate, once armed with the teachings provided herein, that the disclosure encompasses a chemokine and a cytokine, such as are well-known in the art, as well as any discovered in the future.

In some embodiments, the cytokine on the aAPC serves as a polypeptide for stimulating Signal 3. It will be understood that in some embodiments, the aAPCs of the disclosure can expand and/or activate T cells by stimulating all three signals involved in T cell development. Signal 1 involves TCR, and can be stimulated with antigens that signal through TCR. Signal 2 can be mediated by several different molecules, including any immune co-stimulatory molecules described herein, such as 4-1BBL. Signal 3 can be transduced via cytokines, such as IL-15. Without being bound by theory, it is thought that the presence of signal 3, for example from a third exogenous polypeptide on the aAPC, in addition to signals 1 and 2, from a first and second exogenous polypeptide, respectively, e.g., an antigen and costimulatory polypeptide as described herein, increases the capacity of the aAPCs to boost the memory T cell population and thereby provide longer efficacy, e.g., efficacy against a relapse of a tumor or re-challenge with an infectious agent. In some embodiments, the polypeptide for stimulating Signal 3 is IL-15. In some embodiments, the aAPC comprises a third exogenous polypeptide that stimulates Signal 3. In one embodiment, the third exogenous polypeptide that stimulates Signal 3 is IL15.

In some embodiments, an engineered erythroid cell, e.g., enucleated cell, comprises one or more (e.g., 2, 3, 4, 5, or more) cytokine receptor subunits from Table 12 or cytokine-binding variants or fragments thereof. In some embodiments, an engineered erythroid cell comprises two or three (e.g., all) cytokine receptor subunits from a single row of Table 12 or cytokine-binding variants or functional fragments thereof. The cytokine receptors can be present on the surface of the erythroid cell. The expressed receptors typically have the wild type human receptor sequence or a variant or fragment thereof that is able to bind and sequester its target ligand. In embodiments, two or more cytokine receptor subunits are linked to each other, e.g., as a fusion protein.

In embodiments, one or more (e.g., 2 or all) of the cytokines are fused to transmembrane domains (e.g., a GPA transmembrane domain or other transmembrane domain described herein), e.g., such that the cytokine is on the surface of the erythroid cell. In embodiments, the erythroid cell further comprises a targeting moiety, e.g., an address moiety or targeting moiety described in WO2007030708, e.g., in pages 34-45 therein, which application is herein incorporated by reference in its entirety.

In some embodiments, the one or more cytokines include or are fused to a membrane anchor. In some embodiments, the membrane anchor is selected from a sequence set forth in Table 3. In some embodiments, the one or more cytokines include or are fused to a leader sequence. In some embodiments, the leader sequence is selected from a sequence set forth in Table 2.

TABLE 12
Cytokines and Receptors

| Name | Cytokine Receptor(s)(Da) and Form |
|---|---|
| Interleukins | |
| IL-1-like | |
| IL-1α | CD121a, CDw121b |
| IL-1β | CD121a, CDw121b |
| IL-1RA | CD121a |
| IL-18 | IL-18Rα, β |
| Common g chain (CD132) | |
| IL-2 | CD25, 122, 132 |
| IL-4 | CD124, 213a13, 132 |
| IL-7 | CD127, 132 |
| IL-9 | IL-9R, CD132 |
| IL-13 | CD213a1, 213a2, |
| IL-15 | IL-15Ra, CD122, 132 |
| IL-21 | IL21R |
| Common b chain (CD131) | |
| IL-3 | CD123, CDw131 |
| IL-5 | CDw125, 131 |
| Also related | |
| GM-CSF | CD116, CDw131 |
| IL-6-like | |
| IL-6 | CD126, 130 |
| IL-11 | IL-11Ra, CD130 |
| Also related | |
| G-CSF | CD114 |
| IL-12 | CD212 |
| IL-35 | IL35R |
| LIF | LIFR, CD130 |
| OSM | OSMR, CD130 |
| IL-10-like | |
| IL-10 | CDw210 |
| IL-20 | IL-20Rα, β |
| Others | |
| IL-14 | IL-14R |
| IL-16 | CD4 |
| IL-17 | CDw217 |
| Interferons | |
| IFN-α | CD118 |
| IFN-β | CD118 |
| IFN-γ | CDw119 |
| TNF | |
| CD154 | CD40 |
| LT-β | LTβR |
| TNF-α | CD120a, b |
| TNF-β (LT-α) | CD120a, b |
| 4-1BBL | CD137 (4-1BB) |
| APRIL | BCMA, TACI |
| CD70 | CD27 |
| CD153 | CD30 |
| CD178 | CD95 (Fas) |
| GITRL | GITR |
| LIGHT | LTbR, HVEM |
| OX40L | OX40 |
| TALL-1 | BCMA, TACI |
| TRAIL | TRAILR1-4 |
| TWEAK | Apo3 |
| TRANCE | RANK, OPG |
| TGF-β | |
| TGF-β1 | TGF-βR1 |
| TGF-β2 | TGF-βR2 |
| TGF-β3 | TGF-βR3 |
| Miscellaneous hematopoietins | |
| Epo | EpoR |
| Tpo | TpoR |
| Flt-3L | Flt-3 |
| SCF | CD117 |
| M-CSF | CD115 |
| MSP | CDw136 |

### Engineered Erythroid Cells

In some aspects, the present disclosure provides an artificial antigen presenting cell (aAPC) engineered to activate T cells, wherein the aAPC comprises an erythroid cell or an enucleated cell, wherein the erythroid cell or enucleated cell presents, e.g. comprises on the cell surface, an exogenous antigenic polypeptide disclosed in Table 1. Thus, the present disclosure encompasses an aAPC comprising a nucleic acid encoding an antigen of interest, as shown in Table 1. In certain embodiments, at least one exogenous antigenic polypeptide is a tumor antigen, an autoimmune disease antigen, a viral antigen, or a bacterial antigen. In some embodiments an enucleated cell is a erythroid cell, for example, that has lost its nucleus through differentiation from an erythrocyte precursor cell. It will be understood, however, that not all enucleated cells are erythroid cells and, accordingly, enucleated cells encompassed herein can also include, e.g., platelets. In some embodiments, enucleated cells are not platelets and are therefore platelet free enucleated cells. In certain aspects of the disclosure, the erythroid cell is a reticulocyte or an erythrocyte (red blood cell (RBC)). Erythrocytes offer a number of advantages over other cells, including being non-autologous (e.g., substantially lack major histocompatibility complex (MHC)), having longer circulation time in a subject (e.g. greater than 30 days), and being amenable to production in large numbers. In certain aspects of the disclosure, the engineered erythroid cells are nucleated.

The erythroid cell optionally further comprises a second, different, exogenous polypeptide. The erythroid cell optionally further comprises second and third, different, exogenous polypeptides. The erythroid cell optionally further comprises a second, third and fourth, different, exogenous polypeptides. The erythroid cell optionally further comprises a second, third, fourth and fifth, different, exogenous polypeptides. In some embodiments, the erythroid cell optionally further comprises 1, 2, 3, 4, 5, 6, 7, 8, 9, 10, 11, 12, 13, 14, 15, 16, 17, 18, 19, 20 or more different, exogenous polypeptides. In some embodiments, the erythroid cell optionally further comprises between 1-100, 1-200 different, exogenous polypeptides.

In certain embodiments, the erythroid cell (e.g. an engineered erythroid cell) comprising an antigen (e.g. an exogenous antigenic polypeptide), can process and present the antigen in the context of an exogenous antigen-presenting polypeptide, e.g. an MHC (where the cell is also transduced with a nucleic acid encoding a MHC class I or class II molecule), wherein the exogenous antigenic polypeptide is specifically bound to the exogenous antigen-presenting polypeptide (e.g. an MHC class I or class II molecule), thereby producing antigen-specific T cells and expanding a population thereof. Therefore, an antigen of interest can be introduced into an aAPC of the disclosure, wherein the aAPC then presents the antigen in the context of the MHC Class I or II complex (e.g., the antigenic polypeptide is specifically bound to the MHC Class I or II complex), i.e., the MHC molecule is “loaded” with the antigen, and the aAPC can be used to produce an antigen-specific T cell. Thus, in some aspects, the present disclosure provides an artificial antigen presenting cell (aAPC) engineered to activate T cells, wherein the aAPC comprises an engineered erythroid cell, wherein the engineered erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide (e.g. an MHC class I or class II molecule).

In other embodiments, the erythroid cell comprises one or more antigens that are not processed and presented by an MHC, that is, the present disclosure provides an artificial antigen presenting cell (aAPC) engineered to activate T cells without MHC restriction. In some embodiments, the present disclosure provides an aAPC including antibodies against CD3 (including single-chain antibodies). In some embodiments, antibodies against CD3 (including single-chain antibodies) are expressed on the aAPC surface. In some embodiments, the present disclosure provides an aAPC including antibodies against CD4 and CD8. In other embodiments, the antibodies against CD4 and CD8 are expressed on the aAPC surface, to activate their respective immune cell populations.

In other aspects, the present disclosure provides an artificial antigen presenting cell (aAPC) engineered to activate T cells, wherein the aAPC comprises an engineered erythroid cell, wherein the engineered erythroid cell presents, e.g. comprises on the cell surface, an exogenous antigenic polypeptide and an exogenous costimulatory polypeptide.

In some aspects, the present disclosure provides, an artificial antigen presenting cell (aAPC) engineered to activate T cells, wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide, wherein the exogenous antigen-presenting polypeptide is an MHC class I polypeptide or single chain fusion or an MHC class II polypeptide or single chain fusion. In some embodiments of the above aspects and embodiments, the engineered erythroid cell is an enucleated erythroid cell.

In other aspects, the present disclosure provides, an artificial antigen presenting cell (aAPC) engineered to activate and expand T cells, wherein the aAPC comprises an engineered erythroid cell, wherein the engineered erythroid cell presents, e.g. comprises on the cell surface, an exogenous antigen-presenting polypeptide, an exogenous antigenic polypeptide, an exogenous costimulatory polypeptide, and an exogenous T cell expansion polypeptide, wherein the exogenous antigenic polypeptide is specifically bound to the exogenous antigen-presenting polypeptide (e.g. an MHC class I or class II molecule).

In some embodiments, the aAPC is capable of activating a T cell contacted with the aAPC.

In another embodiment, stimulating comprises activation of CD8+ T cells, activation of CD4+ T cells, stimulation of cytotoxic activity of T cells, stimulation of cytokine secretion by T cells, and/or any combination thereof.

In some embodiments of the above aspects and embodiments, the engineered erythroid cell is an enucleated cell.

As another example, the exogenous polypeptides comprise a T cell activating ligand and an agent which inhibits an immune inhibitory molecule (e.g., an immune inhibitory receptor), e.g. CD80 and anti-PD1, in an immuno-oncology setting. In another embodiment, one agent is an activating 4-1BBL, or fragment or variant thereof, and a second agent an antibody molecule that blocks PD1 signaling (e.g., an antibody molecule to PD1 or PD-L1). Thus, in embodiments, a target T cell is both activated and prevented from being repressed.

In some embodiments the objective is to activate or to inhibit T cells. To ensure that T cells are preferentially targeted over other immune cells that may also express either activating or inhibitory receptors as described herein, one of the exogenous polypeptides on the erythroid cell may comprise a targeting moiety, e.g., an antibody molecule that binds the T cell receptor (TCR) or another T cell marker. Targeting moieties are described in more detail hereinbelow. In some embodiments, a specific T cell subtype or clone may be enhanced or inhibited. In some embodiments, one or more of the exogenous polypeptides on the erythroid cell is a peptide-MHC molecule that will selectively bind to a T cell receptor in an antigen-specific manner.

In some aspects, the present disclosure provides an artificial antigen presenting cell (aAPC) engineered to suppress T cell activity, wherein the aAPC comprises an erythroid cell, wherein the erythroid cell presents, e.g. comprises on the cell surface, an exogenous antigen-presenting polypeptide, an exogenous antigenic polypeptide and at least one exogenous co-inhibitory polypeptide disclosed in Table 7, wherein the exogenous antigenic polypeptide is specifically bound to the exogenous antigen-presenting polypeptide (e.g. an MHC class I or class II molecule).

In other aspects, the present disclosure provides an artificial antigen presenting cell (aAPC) engineered to suppress T cell activity, wherein the aAPC comprises an erythroid cell, wherein the erythroid cell presents, e.g. comprises on the cell surface, an exogenous antigen-presenting polypeptide, an exogenous antigenic polypeptide disclosed in Table 1, and at least one exogenous co-inhibitory polypeptide, wherein the exogenous antigenic polypeptide is specifically bound to the exogenous antigen-presenting polypeptide (e.g. an MHC class I or class II molecule).

In some aspects, the present disclosure provides an artificial antigen presenting cell (aAPC) engineered to suppress T cell activity, wherein the aAPC comprises an erythroid cell, wherein the erythroid cell presents, e.g. comprises on the cell surface, an exogenous antigen-presenting polypeptide, an exogenous antigenic polypeptide, and at least one metabolite-altering polypeptide, wherein the exogenous antigenic polypeptide is specifically bound to the exogenous antigen-presenting polypeptide (e.g. an MHC class I or class II molecule).

In other aspects, the present disclosure provides, an artificial antigen presenting cell (aAPC) engineered to suppress T effector cells, wherein the aAPC comprises an engineered erythroid cell, wherein the engineered erythroid cell presents, e.g. comprises on the cell surface, an exogenous antigen-presenting polypeptide, an exogenous antigen, an exogenous proliferation inhibitor, and an exogenous amino acid-depleting polypeptide, wherein the exogenous antigenic polypeptide is specifically bound to the exogenous antigen-presenting polypeptide (e.g. an MHC class I or class II molecule).

In some embodiments, the aAPC is capable of suppressing T cells contacted with the aAPC. In other embodiments, the aAPC is capable of suppressing a T cell that interacts with the aAPC. In further embodiments, the suppressing comprises inhibition of proliferation of a T cell, anergizing of a T cell, or induction of apoptosis of a T cell.

In some aspects, the present disclosure provides, an artificial antigen presenting cell (aAPC) engineered to activate a regulatory T cell (Treg cell), wherein the aAPC comprises an erythroid cell, wherein the erythroid cell presents, e.g. comprises on the cell surface, an exogenous antigen-presenting polypeptide and an exogenous antigenic polypeptide, wherein the exogenous antigenic polypeptide is specifically bound to the exogenous antigen-presenting polypeptide (e.g. an MHC class I or class II molecule). In some embodiments, the aAPC further presents, e.g. comprises on the cell surface, an exogenous Treg expansion polypeptide.

In certain embodiments, the T cell of any one of the aspects and embodiments presented herein is a CD4+ T cell or a CD8+ T cell.

In some embodiments, the erythroid cell comprises an exogenous polypeptide (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides), wherein the erythroid cell optionally further comprises a second exogenous polypeptide (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) is an exogenous polypeptide described herein.

In some embodiments of the above aspects and embodiments, the engineered erythroid cell is an enucleated cell.

The present disclosure should also be construed to encompass “mutants,” “derivatives,” and “variants” of the exogenous polypeptides described herein (or of the DNA encoding the same) which mutants, derivatives and variants are costimulatory ligands, cytokines, antigens (e.g., tumor cell, viral, and other antigens), which are altered in one or more amino acids (or, when referring to the nucleotide sequence encoding the same, are altered in one or more base pairs) such that the resulting peptide (or DNA) is not identical to the sequences recited herein, but has the same biological property as the peptides disclosed herein, in that the peptide has biological/biochemical properties of a costimulatory ligand, cytokine, antigen, and the like, of the present invention (e.g., expression by an aAPC where contacting the aAPC expressing the protein with a T cell, mediates proliferation of, or otherwise affects, the T cell). Any number of procedures may be used for the generation of mutant, derivative or variant forms of a protein of the invention using recombinant DNA methodology well known in the art such as, for example, that described in Sambrook and Russell (2001, Molecular Cloning, A Laboratory Approach, Cold Spring Harbor Press, Cold Spring Harbor, N.Y.), and Ausubel et al. (2002, Current Protocols in Molecular Biology, John Wiley & Sons, NY). Procedures for the introduction of amino acid changes in a protein or polypeptide by altering the DNA sequence encoding the polypeptide are well known in the art and are also described in these, and other, treatises.

The present disclosure contemplates that functional fragments or variants thereof of the proteins listed in Tables 1-24 can be made and screened for similar activity as described herein and would be equivalent hereunder if expressed in engineered erythroid cells as described herein.

The skilled artisan would appreciate, once armed with the teachings provided herein, that the aAPC of the disclosure is not limited in any way to any particular antigen, cytokine, costimulatory ligand, antibody that specifically binds a costimulatory molecule, and the like. Rather, the disclosure encompasses an aAPC comprising numerous molecules, either all expressed under the control of a single promoter/regulatory sequence or under the control of more than one such sequence. Moreover, the disclosure encompasses administration of one or more aAPC of the disclosure where the various aAPCs encode different molecules. That is, the various molecules (e.g., costimulatory ligands, antigens, cytokines, and the like) can work in cis (i.e., in the same aAPC and/or encoded by the same contiguous nucleic acid or on separate nucleic acid molecules within the same aAPC) or in trans (i.e., the various molecules are expressed by different aAPCs).

### Engineered Erythroid Cells Comprising Three or More Exogenous Polypeptide

In embodiments, an engineered erythroid cell described herein comprises three or more, e.g., at least 4, 5, 6, 7, 8, 9, 10, 11, 12, 13, 14, 15, 16, 17, 18, 19, 20, 50, 100, 200, 500, or 1000 exogenous polypeptides. In embodiments, a population of erythroid cells described herein comprises three or more, e.g., at least 4, 5, 6, 7, 8, 9, 10, 11, 12, 13, 14, 15, 16, 17, 18, 19, 20, 50, 100, 200, 500, 1000, 2000, or 5000 exogenous polypeptides, e.g., wherein different erythroid cells in the population comprise different exogenous polypeptides or wherein different erythroid cells in the population comprise different pluralities of exogenous polypeptides.

### Tiling

In some embodiments, the first exogenous antigenic polypeptide and the second exogenous antigenic polypeptide have amino acid sequences which overlap. In certain embodiments, an aAPC is engineered to activate T cells, wherein the aAPC comprises an erythroid cell, wherein the erythroid cell presents, e.g. comprises on the cell surface, a first exogenous antigenic polypeptide and a second exogenous antigenic polypeptide, and wherein the first exogenous antigenic polypeptide and the second exogenous antigenic polypeptide have amino acid sequences which overlap by at least 2 amino acids. In some embodiments, the overlap is between 2 amino acids and 23 amino acids, for example the overlap is 2, 3, 4, 5, 6, 7, 8, 9, 10, 11, 12, 13, 14, 15, 16, 17, 18, 19, 20, 21, 22 or 23 amino acids. In some embodiments, the exogenous antigenic polypeptide is between 8-10 amino acids in length, and the overlap is between 6-8 amino acids. In some embodiments, the exogenous antigenic polypeptide is between 14-20 amino acids in length, and the overlap is between 12-18 amino acids. Tiling polypeptides in this way provides broader recognition of antigen. In some embodiments of the above aspects and embodiments, the engineered erythroid cell is an enucleated cell.

Methods for tiling polypeptides are known in the art, and are described, for example in Harding et al., which describes the development and testing of 15 mer polypeptides, overlapping by 12 amino acids, that were tested in a human CD4+ T-cell-based proliferative assay (Molecular Cancer Therapeutics, November 2005, Volume 4, Issue 11, incorporated by reference in its entirety herein). Sticker, et al. describes a human cell-based method to identify functional CD4(+) T-cell epitopes in any protein (J Immunol Methods. 2003 Oct. 1; 281(1-2): 95-108, incorporated by reference in its entirety herein).

### Modifications

One or more of the exogenous proteins may have post-translational modifications characteristic of eukaryotic cells, e.g., mammalian cells, e.g., human cells. In some embodiments, one or more (e.g., 2, 3, 4, 5, 6, 7, 8, 9, 10, or more) of the exogenous proteins are glycosylated, phosphorylated, or both. In vitro detection of glycoproteins can be accomplished on SDS-PAGE gels and Western Blots using a modification of Periodic acid-Schiff (PAS) methods. Cellular localization of glycoproteins can be accomplished utilizing lectin fluorescent conjugates known in the art. Phosphorylation may be assessed by Western blot using phospho-specific antibodies.

Post-translation modifications also include conjugation to a hydrophobic group (e.g., myristoylation, palmitoylation, isoprenylation, prenylation, or glypiation), conjugation to a cofactor (e.g., lipoylation, flavin moiety (e.g., FMN or FAD), heme C attachment, phosphopantetheinylation, or retinylidene Schiff base formation), diphthamide formation, ethanolamine phosphoglycerol attachment, hypusine formation, acylation (e.g. O-acylation, N-acylation, or S-acylation), formylation, acetylation, alkylation (e.g., methylation or ethylation), amidation, butyrylation, gamma-carboxylation, malonylation, hydroxylation, iodination, nucleotide addition such as ADP-ribosylation, oxidation, phosphate ester (O-linked) or phosphoramidate (N-linked) formation, (e.g., phosphorylation or adenylylation), propionylation, pyroglutamate formation, S-glutathionylation, S-nitrosylation, succinylation, sulfation, ISGylation, SUMOylation, ubiquitination, Neddylation, or a chemical modification of an amino acid (e.g., citrullination, deamidation, eliminylation, or carbamylation), formation of a disulfide bridge, racemization (e.g., of proline, serine, alanine, or methionine). In embodiments, glycosylation includes the addition of a glycosyl group to arginine, asparagine, cysteine, hydroxylysine, serine, threonine, tyrosine, or tryptophan, resulting in a glycoprotein. In embodiments, the glycosylation comprises, e.g., O-linked glycosylation or N-linked glycosylation.

In some embodiments, one or more of the exogenous polypeptides is a fusion protein, e.g., is a fusion with an endogenous red blood cell protein or fragment thereof, e.g., a transmembrane protein, e.g., GPA or a transmembrane fragment thereof. In some embodiments, one or more of the exogenous polypeptides is fused with a domain that promotes dimerization or multimerization, e.g., with a second fusion exogenous polypeptide, which optionally comprises a dimerization domain. In some embodiments, the dimerization domain comprises a portion of an antibody molecule, e.g., an Fc domain or CH3 domain. In some embodiments, the first and second dimerization domains comprise knob-in-hole mutations (e.g., a T366Y knob and a Y407T hole) to promote heterodimerization.

### Copy Number

In some embodiments, the first exogenous polypeptide and the second exogenous polypeptide have an abundance ratio of about 1:1, from about 2:1 to 1:2, from about 5:1 to 1:5, from about 10:1 to 1:10, from about 20:1 to 1:20, from about 50:1 to 1:50, from about 100:1 to 1:100 by weight or by copy number.

In some embodiments, the engineered erythroid cell comprises at least at least 10 copies, 100 copies, 1,000 copies, 5,000 copies 10,000 copies, 25,000 copies, 50,000 copies, or 100,000 copies of each of the first exogenous polypeptide and the second exogenous polypeptide. In some embodiments, the copy number of the first exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the second exogenous polypeptide. In some embodiments, the copy number of the second exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the first exogenous polypeptide. In some embodiments, the engineered erythroid cell is an enucleated cell. In some embodiments, the engineered erythroid cell is a nucleated cell.

In some embodiments, the first exogenous polypeptide comprises between about 50,000 to about 600,000 copies of the first exogenous polypeptide, for example about 50,000, 60,000, 60,000, 80,000, 90,000, 100,000, 110,000, 120,000, 130,000, 140,000, 150,000, 155,000, 160,000, 165,000, 170,000, 175,000, 180,000, 185,000, 190,000, 195,000, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000, 300,000, 305,000, 310,000, 315,000, 320,000, 325,000, 330,000, 335,000, 340,000, 345,000, 350,000, 355,000, 360,000, 365,000, 370,000, 375,000, 380,000, 385,000, 390,000, 395,000, 400,000, 450,000, 500,000, 550,000, 600,000 copies of the first polypeptide. In some embodiments, the engineered erythroid cell comprises between about 50,000-600,000, between about 100,000-600,000, between about 100,000-500,000, between about 100,000-400,000, between about 100,000-150,000, between about 150,000-300,000, or between 150,000-200,000 copies of the first exogenous polypeptide. In some embodiments, the engineered erythroid cell comprises at least about 75,000 copies of the first exogenous polypeptide. In some embodiments, the engineered erythroid cell comprises at least about 100,000 copies of the first exogenous polypeptide. In some embodiments, the engineered erythroid cell comprises at least about 125,000 copies of the first exogenous polypeptide. In some embodiments, the engineered erythroid cell comprises at least about 150,000 copies of the first exogenous polypeptide. In some embodiments, the engineered erythroid cell comprises at least about 175,000 copies of the first exogenous polypeptide. In some embodiments, the engineered erythroid cell comprises at least about 200,000 copies of the first exogenous polypeptide. In some embodiments, the engineered erythroid cell comprises at least about 250,000 copies of the first exogenous polypeptide. In some embodiments, the engineered erythroid cell comprises at least about 300,000 copies of the first exogenous polypeptide. In some embodiments, the engineered erythroid cell comprises at least about 400,000 copies of the first exogenous polypeptide. In some embodiments, the engineered erythroid cell comprises at least about 500,000 copies of the first exogenous polypeptide. In some embodiments, the second exogenous polypeptide comprises between about 50,000 to about 600,000 copies of the second exogenous polypeptide, for example about 50,000, 60,000, 60,000, 80,000, 90,000, 100,000, 110,000, 120,000, 130,000, 140,000, 150,000, 155,000, 160,000, 165,000, 170,000, 175,000, 180,000, 185,000, 190,000, 195,000, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000, 300,000, 305,000, 310,000, 315,000, 320,000, 325,000, 330,000, 335,000, 340,000, 345,000, 350,000, 355,000, 360,000, 365,000, 370,000, 375,000, 380,000, 385,000, 390,000, 395,000, 400,000, 450,000, 500,000, 550,000, 600,000 copies of the second polypeptide. In some embodiments, the engineered erythroid cell comprises between about 50,000-600,000, between about 100,000-600,000, between about 100,000-500,000, between about 100,000-400,000, between about 100,000-150,000, between about 150,000-300,000, or between 150,000-200,000 copies of the second exogenous polypeptide. In some embodiments, the engineered erythroid cell comprises at least about 75,000 copies of the second exogenous polypeptide. In some embodiments, the engineered erythroid cell comprises at least about 100,000 copies of the second exogenous polypeptide. In some embodiments, the engineered erythroid cell comprises at least about 125,000 copies of the second exogenous polypeptide. In some embodiments, the engineered erythroid cell comprises at least about 150,000 copies of the second exogenous polypeptide. In some embodiments, the engineered erythroid cell comprises at least about 175,000 copies of the second exogenous polypeptide. In some embodiments, the engineered erythroid cell comprises at least about 200,000 copies of the second exogenous polypeptide. In some embodiments, the engineered erythroid cell comprises at least about 250,000 copies of the second exogenous polypeptide. In some embodiments, the engineered erythroid cell comprises at least about 300,000 copies of the second exogenous polypeptide. In some embodiments, the engineered erythroid cell comprises at least about 400,000 copies of the second exogenous polypeptide. In some embodiments, the engineered erythroid cell comprises at least about 500,000 copies of the second exogenous polypeptide.

In some embodiments of the above aspects and embodiments, the engineered erythroid cell is an enucleated cell. In some embodiments of the above aspects and embodiments, the engineered erythroid cell is a nucleated cell.

### In Vivo Half-Life

In some embodiments, an exogenous polypeptide described herein, when included in or on an engineered erythroid cell or an enucleated cell and administered to a subject, exhibits a prolonged in vivo half-life as compared to a corresponding exogenous polypeptide that is administered by itself (i.e., not on or in a cell described herein). In some embodiments, the exogenous polypeptide has an in vivo half-life that is longer than the in vivo half-life of a corresponding exogenous polypeptide that is administered by itself, or the in vivo half-life of a corresponding pegylated version of the exogenous polypeptide that is administered by itself. In some embodiments, the exogenous polypeptide has an in vivo half-life of between about 24 hours and 240 days (e.g., 24 hours, 36 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132, days, 133 days, 134 days, 135 days, 136 days, 137 days, 138 days, 139 days, 140 days, 141 days, 142 days, 143 days, 144 days, 145 days, 146 days, 147 days, 148 days, 149 days, 150 days, 151 days, 152 days, 153 days, 154 days, 155 days, 156 days, 157 days, 158 days, 159 days, 160 days, 161 days, 162 days, 163 days, 164 days, 165 days, 166 days, 167 days, 168 days, 169 days, 170 days, 171 days, 172 days, 173 days, 174 days, 175 days, 176 days, 177 days, 178 days, 179 days, 180 days, 181 days, 182 days, 183 days, 184 days, 185 days, 186 days, 187 days, 188 days, 189 days, 190 days, 191 days, 192 days, 193 days, 194 days, 195 days, 196 days, 197 days, 198 days, 919 days, 200 days, 201 days, 202 days, 203 days, 204 days, 205 days, 206 days, 207 days, 208 days, 209 days, 210 days, 211 days, 212 days, 213 days, 214 days, 215 days, 216 days, 217 days, 218 days, 219 days, 220 days, 221 days, 222 days, 223 days, 224 days, 225 days, 226 days, 227 days, 228 days, 229 days, 230 days, 231 days, 232, days, 233 days, 234 days, 235 days, 236 days, 237 days, 238 days, 239 days, or 240 days. In some embodiments, the exogenous polypeptide has an in vivo half-life of greater than 1 day, 2 days, 3 days, 5 days, 10 days, 25 days, 50 days, 75 days, 100 days, 125 days, 150 days, 175 days, 200 days, 225 days, 235 days, or 250 days. In some embodiments, the exogenous polypeptide has an in vivo half-life of 1 week, 2 weeks, 3 weeks, 1 month, 2 months, 3 months, 4 months, 5 months, 6 months, 7 months, 8 months, 9 months, 10 months, 11 months, a year, or more.

In some embodiments, the aAPC of the present disclosure resides in circulation after administration to a subject for at least about 1 day to about 240 days (e.g., for at least about 1 day, 2 days, 3 days, 4 day, 5 days, 6 days, 7 days, 8 days, 9 days, 10 days, 11 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132 days, 133 days, 134 days, 135 days, 136 days, 137 days, 138 days, 139 days 140 days, 141 days, 142 days, 143 days, 144 days, 145 days, 146 days, 147 days, 148 days, 149 days, 150 days, 151 days, 152 days, 153 days, 154 days, 155 days, 156 days, 157 days, 158 days, 159 days, 160 days, 161 days, 162 days, 163 days, 164 days, 165 days, 166 days, 167 days, 168 days, 169 days, 170 days, 171 days, 172 days, 173 days, 174 days, 175 days, 176 days, 177 days, 178 days, 179 days, 180 days, 181 days, 182 days, 183 days, 184 days, 185 days, 186 days, 187 days, 188 days, 189 days, 190 days, 191 days, 192 days, 193 days, 194 days, 195 days, 196 days, 197 days, 198 days, 199 days, 200 days, 201 days, 202 days, 203 days, 204 days, 205 days, 206 days, 207 days, 208 days, 209 days, 210 days, 211 days, 212 days, 213 days, 214 days, 215 days, 216 days, 217 days, 218 days, 219 days, 220 days, 221 days, 222 days, 223 days, 224 days, 225 days, 226 days, 227 days, 228 days, 229 days, 230 days, 231 days, 232 days, 233 days, 234 days, 235 days, 236 days, 237 days, 238 days, 239 days, or 240 days.

In some embodiments, the aAPC of the present disclosure presents the antigenic polypeptide during circulation of aAPCs through the vasculature. In some embodiments, the aAPC of the present disclosure presents the antigenic polypeptide in the spleen.

### Gene Editing

In some aspects, the disclosure features a method of making an immunologically compatible artificial antigen presenting cell (aAPC), wherein the aAPC comprises an engineered erythroid cell that expresses an exogenous antigenic polypeptide, the method comprising contacting the aAPC with a nuclease and at least one gRNA which cleave an endogenous MHC nucleic acid, wherein the endogenous MHC nucleic acid is repaired by a gene editing pathway and results in a decrease in the level of expression of the endogenous MHC nucleic acid, thereby making the immunologically compatible aAPC. In some embodiments, the engineered erythroid cell is an enucleated cell. In some embodiments, the engineered erythroid cell is a nucleated cell.

In some embodiments, a cell is genetically modified using a nuclease that is targeted to one or more selected DNA sequences. Such methods may be used to induce precise cleavage at selected sites in endogenous genomic loci. Genetic engineering in which DNA is inserted, replaced, or removed from a genome, e.g., at a defined location of interest, using targetable nucleases, may be referred to as “genome editing”. Examples of such nucleases include zinc-finger nucleases (ZFNs), Transcription activator-like effector nuclease (TALENs), engineered meganuclease homing endonucleases, and RNA directed nucleases such as CRISPR (clustered regularly interspaced short palindromic repeats)-associated (Cas) nucleases, e.g., derived from type II bacterial CRISPR/Cas systems (e.g., Cas9).

In some embodiments, an alteration is first introduced using CRISPR (i.e. increasing endogenous expression of MHCI). Then, the antigen for presentation is also introduced via CRISPR and processed internally.

In some embodiments the nuclease comprises a DNA cleavage domain and a DNA binding domain (DBD) that targets the nuclease to a particular DNA sequence, thereby allowing the nuclease to be used to engineer genomic alterations in a sequence-specific manner. The DNA cleavage domain may create a double-stranded break (DSB) or nick at or near the sequence to which it is targeted. ZFNs comprise DBDs selected or designed based on DBDs of zinc finger (ZF) proteins. DBDs of ZF proteins bind DNA in a sequence-specific manner through one or more zinc fingers, which are regions of amino acid sequence whose structure is stabilized through coordination of a zinc ion. TALENs comprise DBDs selected or designed based on DBDs of transcription activator-like (TAL) effectors (TALEs) of Xanthomonas spp. ZFN or TALEN dimers induce targeted DNA DSBs that stimulate DNA damage response pathways. The binding specificity of the designed zinc-finger domain directs the ZFN to a specific genomic site. TALEs contain multiple 33-35-amino-acid repeat domains, each of which recognizes a single base pair. Like ZFNs, TALENs induce targeted DSBs that activate DNA damage response pathways and enable custom alterations. The DNA cleavage domain of an engineered site-specific nuclease may comprise a catalytic domain from a naturally occurring endonuclease such as the Fok1 endonuclease or a variant thereof. In some embodiments Fok1 cleavage domain variants with mutations designed to improve cleavage specificity and/or cleavage activity may be used (see, e.g., Guo, J., et al. (2010) Journal of Molecular Biology 400 (1): 96-107; Doyon, Y., et al., (2011) Nature Methods 8: 74-79. Meganucleases are sequence-specific endonucleases characterized by a large recognition site (double-stranded DNA sequences of 12 to about 40 base pairs). The site generally occurs no more than once in a given genome. The specificity of a meganuclease can be changed by introducing changes sequence of the nuclease (e.g., in the DNA binding domain) and then selecting functional enzymes capable of cleaving variants of the natural recognition site or by associating or fusing protein domains from different nucleases.

In some embodiments, an RNA directed nuclease may be used to perform genome editing. For example, the use of CRISPR/Cas-based systems is contemplated. In some embodiments a Cas nuclease, such as Cas9 (e.g., Cas9 of Streptococcus pyogenes, Streptococcus thermophiles, or Neisseria meningitides, or a variant thereof), is introduced into cells along with a guide RNA comprising a sequence complementary to a sequence of interest (the RNA is sometimes termed a single guide RNA). The region of complementarity may be, e.g., about 20 nucleotides long. The Cas nuclease, e.g., Cas9, is guided to a particular DNA sequence of interest by the guide RNA. The guide RNA may be engineered to have complementarity to a target sequence of interest in the genome, e.g., a sequence in any gene or intergenic region of interest. The nuclease activity of the Cas protein, e.g., Cas9, cleaves the DNA, which can disable the gene, or cut it apart, allowing a different DNA sequence to be inserted. In some embodiments multiple sgRNAs comprising sequences complementary to different genes, e.g., 2, 3, 4, 5, or more genes, are introduced into the same cell sequentially or together. In some embodiments alterations in multiple genes may thereby be generated in the same step.

In general, use of nuclease-based systems for genetic engineering, e.g., genome editing, entails introducing a nuclease into cells and maintaining the cells under conditions and for a time appropriate for the nuclease to cleave the cell's DNA. In the case of

CRISP/Cas systems, a guide RNA is also introduced. The nuclease is typically introduced into the cell by introducing a nucleic acid encoding the nuclease. The nucleic acid may be operably linked to a promoter capable of directing expression in the cell and may be introduced into the cell in a plasmid or other vector. In some embodiments mRNA encoding the nuclease may be introduced. In some embodiments the nuclease itself may be introduced. sgRNA may be introduced directly (by methods such as transfection) or by expressing it from a nucleic acid construct such as an expression vector. In some embodiments a sgRNA and Cas protein are expressed from a single expression vector that has been introduced into the cell or, in some embodiments, from different expression vectors. In some embodiments multiple sgRNAs comprising sequences complementary to different genes, e.g., 2, 3, 4, 5, or more genes, are introduced into the same cell individually or together as RNA or by introducing one or more nucleic acid constructs encoding the sgRNAs into the cell for intracellular transcription.

Upon cleavage by a nuclease, a target locus (e.g., in the genome of a cell) may undergo one of two major pathways for DNA damage repair, namely non-homologous end joining (NHEJ) or homology-directed repair (HDR). In the absence of a suitable repair template comprising sufficient homology to the sequences flanking the cleavage site to stimulate HDR (see discussion below), DSBs are re-ligated through NHEJ, which can result in an insertion or deletion. NHEJ can be used, for example, to engineer gene knockouts or generate proteins with altered activity. For example, an insertion or deletion in an exon can lead to a frameshift mutation or premature stop codon. Two or more DSBs can be generated in order to produce larger deletions in the genome.

In some embodiments a nucleic acid (e.g., a plasmid or linear DNA) comprising a sequence of interest to be inserted into the genome at the location of cleavage is introduced into a cell in addition to a nuclease. In some embodiments a sequence of interest is inserted into a gene. The sequence of interest may at least in part replace the gene. In some embodiments the nucleic acid comprises sequences that are homologous to the sequences flanking the cleavage site, so that homology-directed repair is stimulated. In some embodiments the nucleic acid contains a desired alteration as compared to a sequence present in the cell's genome at or near the site of cleavage. A nucleic acid comprising a sequence to be at least in part introduced into the genome, e.g., a nucleic acid sequence comprising homologous sequence(s) and a desired alteration may be referred to as a “donor sequence”. The donor sequence may become at least in part physically into integrated the genome at the site of a break or may be used as a template for repair of the break, resulting in the introduction of all or part of the nucleotide sequence present in the donor into the genome of the cell. Thus, a sequence in a cell's genome can be altered and, in certain embodiments, can be converted into a sequence present in a donor nucleic acid. In some embodiments the donor sequence may be contained in a circular DNA (e.g. a plasmid), a linear double-stranded DNA (e.g., a linearized plasmid or a PCR product), or single-stranded DNA, e.g., a single-stranded oligonucleotide. In some embodiments the donor sequence has between about 10-25 bp and about 50-100 bp of homology to either side or each side of the target site in the genome. In some embodiments a longer homologous sequence may be used, e.g., between about 100-500 bp up to about 1-2 kB, or more. In some embodiments an alteration is introduced into one allele of a gene. In some embodiments a first alteration is introduced into one allele of a gene, and a different alteration is introduced into the other allele. In some embodiments the same alteration is introduced into both alleles. In some embodiments two alleles or target sites (or more) may be genetically modified in a single step. In some embodiments two alleles or target sites (or more) may be genetically modified in separate steps.

Methods of designing, generating and using ZFNs and/or TALENs are described in, e.g., WO2011097036; Urnov, F D, et al., Nature Reviews Genetics (2010), 11: 636-646; Miller J C, et al., Nat Biotechnol. (2011) 29(2): 143-8; Cermak, T., et al. Nucleic Acids Research (2011) 39 (12): e82, Sanjana, N. E. et al. A transcription activator-like effector toolbox for genome engineering. Nat Protoc 7, 171-192 (2012) and references in any of the foregoing. ZFN, TALEN, and CRISPR/Cas-based methods for genome engineering are reviewed in Gaj, T., et al., Trends Biotechnol. 2013 July; 31(7):397-405. Epub 2013 May 9. Use of CRISPR/Cas systems in genome engineering is described in, e.g., Cong L, et al. Multiplex genome engineering using CRISPR/Cas systems. Science. 2013; 339(6121):819-23; Mali P, et al., RNA-guided human genome engineering via Cas9. Science. 2013; 339(6121):823-6; Wang, H. et al. One-step generation of mice carrying mutations in multiple genes by CRISPR/Cas-mediated genome engineering. Cell 153, 910-918 (2013); Ran, F. A. et al. Double Nicking by RNA-Guided CRISPR Cas9 for Enhanced Genome Editing Specificity. Cell 154, 1380-1389 (2013); Mali, P., et al., Nat Methods. 2013; 10(10):957-63; Ran, F A, Nat Protoc. 2013; 8(11):2281-308). In some embodiments a nuclease that cleaves only one strand of dsDNA (a nickase) may be used to stimulate HDR without activating the NHEJ repair pathway. Nickases may be created by inactivating the catalytic activity of one nuclease monomer in the ZFN or TALEN dimer required for double stranded cleavage or inactivating a catalytic domain of a Cas protein. For example, mutations of one of the catalytic residues (D10 in the RuvC nuclease domain and H840 in the HNH nuclease domain), e.g., to alanines (D10A, H840A) convert Cas9 into DNA nickases.

In some embodiments, a CRISP/Cas based system may be used to modulate gene expression. For example, coexpression of a guide RNA with a catalytically inactive Cas9 lacking endonuclease activity generates a DNA recognition complex that can specifically interfere with transcriptional elongation, RNA polymerase binding, or transcription factor binding. This system, sometimes referred to CRISPR interference (CRISPRi), can efficiently repress expression of targeted genes in mammalian cells (Qi, S., et al., Cell, 2013; 152(5): 1173-83; Larson, M H, et al, Nat Protoc. 2013; 8(11):2180-96). By attaching any of a variety of effector domains to a catalytically inactive Cas9 one can create a chimeric Cas9 protein that can be used to achieve sequence-specific control over gene expression and/or DNA modification. Suitable effector domains include, e.g., a transcriptional activation domain (such as those comprising the VP16 transactivation domain, e.g., VP64), a transcriptional coactivation domain, a transcriptional inhibitory or co-inhibitory domain, a protein-protein interaction domain, an enzymatic domain, etc. A guide RNA guides the chimeric Cas9 protein to a site of interest in the genome (e.g., in or near an expression control element such as a promoter), whereby the effector domain exerts an effect such as activating or inhibiting transcriptional activity (see, e.g., Gilbert L A, et al. Cell. 2013; 154(2):442-51; Maeder M L, et al., Nat Methods, 2013; 10(10):977-9), Appropriate effector domains may be any of those present in naturally occurring proteins that are capable of performing the function of interest (e.g., inhibiting or activating transcription).

Cells that have been subjected to a genetic engineering process may be selected or analyzed to identify or isolate those that express a desired recombinant gene product or lack expression of an endogenous gene that has been disabled via genetic engineering or have any desired genetic alteration. For example, in some embodiments the donor sequence or vector used to deliver the donor sequence may comprise a selectable marker, which may be used to select cells that have incorporated at least a portion of the donor sequence comprising the selectable marker into their genome. In some embodiments selection is not used. In some embodiments cells may be screened, e.g., by Southern blot to identify those cells or clones that have a desired genetic alteration. If desired, cells may be tested for expression level or activity of a recombinant gene product or endogenous gene product or for one or more functional properties associated with or conferred by a recombinant or endogenous gene product, or any other criteria of interest. Suitable methods of analysis are known to those of ordinary skill in the art and include, e.g., Western blot, flow cytometry, FAGS, immunofluorescence microscopy, ELISA assays, affinity-based methods in which cells are contacted with an agent capable of binding to a protein of interest that labels or retains cells that express the protein, etc. Functional assays may be selected based on the identity of the recombinant gene product, endogenous gene product, and/or function or property of interest. For example, a functional property may be ability to bind to an antigen of interest or ability to exert cytotoxicity towards target cells that express an antigen of interest. Cells may be analyzed, e.g., by PGR, Southern blotting, or sequencing, to determine the number of inserted DNA sequences, their location, and/or to determine whether desired genomic alterations have occurred. One or more cells that have desired alteration(s), expression level, and/or functional properties may be identified, propagated, expanded. The cells or their descendants may be used to generate a cell line, subjected to sortagging, and/or stored for future use.

### Populations of Engineered Erythroid Cells

In one aspect, the invention features cell populations comprising the engineered erythroid cells of the invention, e.g., a plurality or population of the engineered erythroid cells. In various embodiments, the engineered erythroid cell population comprises predominantly enucleated cells, predominantly nucleated cells, or a mixture of enucleated and nucleated cells. In such cell populations, the enucleated cells can comprise reticulocytes, erythrocytes, or a mixture of reticulocytes and erythrocytes. In some embodiments, the enucleated cells are reticulocytes. In some embodiments, the enucleated cells are erythrocytes.

In some embodiments, the engineered erythroid cell population consists essentially of enucleated cells. In some embodiments, the engineered erythroid cell population comprises predominantly or substantially enucleated cells. For example, In some embodiments, the population of engineered erythroid cells comprises at least about 80% or more enucleated cells. In some embodiments, the population provided herein comprises at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99, or about 100% enucleated cells. In some embodiments, the population provided herein comprises greater than about 80% enucleated cells. In some embodiments, the population of engineered erythroid cells comprises greater than about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% enucleated cells. In some embodiments, the population of engineered erythroid cells comprises between about 80% and about 100% enucleated cells, for example between about 80% and about 95%, about 80% and about 90%, about 80% and about 85%, about 85% and about 100%, about 85% and about 95%, about 85% and about 90%, about 90% and about 100%, about 90% and about 95%, or about 95% and about 100% of enucleated cells.

In some embodiments, the population of engineered erythroid cells comprises less than about 20% nucleated cells. For example, in embodiments, the population of engineered erythroid cells comprises less than about 1%, about 2%, about 3%, about 5%, about 6%, about 7%, about 8%, about 9%, about 10%, about 11%, about 12%, about 13%, about 14%, about 15%, about 16%, about 17%, about 18%, about 19%, or less than about 20% nucleated cells. In some embodiments, the population of engineered erythroid cells comprises less than about 1% nucleated cells. In some embodiments, the population of engineered erythroid cells comprises less than about 2% nucleated cells. In some embodiments, the population of engineered erythroid cells comprises less than about 3% nucleated cells. In some embodiments, the population of engineered erythroid cells comprises less than about 4% nucleated cells. In some embodiments, the population of engineered erythroid cells comprises less than about 5% nucleated cells. In some embodiments, the population of engineered erythroid cells comprises less than about 10% nucleated cells. In some embodiments, the population of engineered erythroid cells comprises less than about 15% nucleated cells. In some embodiments, the population of engineered erythroid cells comprises between 0% and 20% nucleated cells. In some embodiments, the populations of engineered erythroid cells comprise between about 0% and 20% nucleated cells, for example between about 0% and 19%, between about 0% and 15%, between about 0% and 10%, between about 0% and 5%, between about 0% and 4%, between about 0% and 3%, between about 0% and 2% nucleated cells, or between about 5% and 20%, between about 10% and 20%, or between about 15% and 20% nucleated cells.

In some embodiments, the disclosure features a population of the engineered erythroid cells of the invention, wherein the population of engineered erythroid cells comprises less than 20% nucleated cells and at least 80% enucleated cells, or comprises less than 15% nucleated cells and at least 85% nucleated cells, or comprises less than 10% nucleated cells and at least 90% enucleated cells, or comprises less than 5% nucleated cells and at least 95% enucleated cells. In some embodiments, the disclosure features populations of the engineered erythroid cells of the invention, wherein the population of engineered erythroid cells comprises about 0% nucleated cells and about 100% enucleated cells, about 1% nucleated cells and about 99% enucleated cells, about 2% nucleated cells and about 98% enucleated cells, about 3% nucleated cells and about 97% enucleated cells, about 4% nucleated cells and about 96% enucleated cells, about 5% nucleated cells and about 95% enucleated cells, about 6% nucleated cells and about 94% enucleated cells, about 7% nucleated cells and about 93% enucleated cells, about 8% nucleated cells and about 92% enucleated cells, about 9% nucleated cells and about 91% enucleated cells, about 10% nucleated cells and about 90% enucleated cells, about 11% nucleated cells and about 89% enucleated cells, about 12% nucleated cells and about 88% enucleated cells, about 13% nucleated cells and about 87% enucleated cells, about 14% nucleated cells and about 86% enucleated cells, about 85% nucleated cells and about 85% enucleated cells, about 16% nucleated cells and about 84% enucleated cells, about 17% nucleated cells and about 83% enucleated cells, about 18% nucleated cells and about 82% enucleated cells, about 19% nucleated cells and about 81% enucleated cells, or about 20% nucleated cells and about 80% enucleated cells.

In another embodiment, the engineered erythroid cell population comprises predominantly or substantially nucleated cells. In some embodiments, the engineered erythroid cell population consists essentially of nucleated cells. In various embodiments, the nucleated cells in the engineered erythroid cell population are erythrocyte (or fully mature red blood cell) precursor cells. In embodiments, the erythrocyte precursor cells are selected from the group consisting of pluripotent hematopoietic stem cells (HSCs), multipotent myeloid progenitor cells, CFU-S cells, BFU-E cells, CFU-E cells, pronormoblasts, basophilic normoblasts, polychromatophilic normoblasts and orthochromatophilic normoblasts.

In some embodiments, the erythrocyte precursor cells, e.g., hematopoietic stem cells, are from an O-negative donor.

In certain embodiments, the population of engineered erythroid cells comprises at least about 10%, at least about 20%, at least about 30%, at least about 40%, at least about 50%, at least about 60%, at least about 70%, at least about 75%, at least about 80%, at least about 85%, at least about 90%, at least about 95%, at least about 98%, at least about 99% or 100% nucleated cells.

It will be understood that during the preparation of the engineered erythroid cells of the invention, some fraction of cells may not become conjugated with an exogenous polypeptide or transduced to express an exogenous polypeptide. Accordingly, in some embodiments, a population of engineered erythroid cells provided herein comprises a mixture of engineered erythroid cells and unmodified erythroid cells, i.e., some fraction of cells in the population will not comprise, present, or express an exogenous polypeptide. For example, a population of engineered erythroid cells can comprise, in various embodiments, at least about 10%, 20%, 30%, 40%, 50%, 60%, 70%, 80%, 90%, 91%, 92%, 93%, 94%, 95%, 96%, 97%, 98% or 99% engineered erythroid cells, wherein the remaining erythroid cells in the population are not engineered. In embodiments, a single unit dose of engineered erythroid cells can comprise, in various embodiments, at least about 10%, 20%, 30%, 40%, 50%, 60%, 70%, 80%, 90%, 91%, 92%, 93%, 94%, 95%, 96%, 97%, 98% or 99% engineered erythroid cells, wherein the remaining erythroid cells in the dose are not engineered.

### III. Methods of Making Artificial Antigen Presenting Cells

Various methods of making aAPCs are contemplated by the present disclosure.

Methods of manufacturing enucleated erythroid cells comprising an exogenous agent (e.g., a polypeptide) are described, e.g., in International Application Publication Nos. WO2015/073587 and WO2015/153102, each of which is incorporated by reference in its entirety.

In some embodiments, hematopoietic progenitor cells, e.g., CD34⁺ hematopoietic progenitor cells (e.g., human (e.g., adult human) or mouse cells), are contacted with a nucleic acid or nucleic acids encoding one or more exogenous polypeptides, and the cells are allowed to expand and differentiate in culture. In some embodiments, the CD34⁺ cells are immortalized, e.g., comprise a human papilloma virus (HPV; e.g., HPV type 16) E6 and/or E7 genes. In some embodiments, the immortalized CD34⁺ hematopoietic progenitor cell is a BEL-A cell line cell (see Trakarnasanga et al. (2017) Nat. Commun. 8: 14750). Additional immortalized CD34⁺ hematopoietic progenitor cells are described in U.S. Pat. Nos. 9,951,350, and 8,975,072. In some embodiments, an immortalized CD34⁺ hematopoietic progenitor cell is contacted with a nucleic acid or nucleic acids encoding one or more exogenous polypeptides, and the cells are allowed to expand and differentiate in culture.

In one aspect, the present disclosure features a method of making an immunologically compatible artificial antigen presenting cell (aAPC), wherein the aAPC comprises an erythroid cell or enucleated cell that presents, e.g. comprises on the cell surface, an exogenous antigenic polypeptide, the method comprising contacting a nucleated cell with a nuclease and at least one gRNA which cleave an endogenous nucleic acid to result in expression of an endogenous antigen-presenting polypeptide, an endogenous anchor polypeptide, or an endogenous costimulatory polypeptide; or to result in inhibition of expression of an endogenous microRNA; introducing an exogenous nucleic acid encoding the exogenous antigenic polypeptide into the nucleated cell; and culturing the nucleated cell under conditions suitable for expression and presentation of the exogenous antigenic polypeptide by the endogenous antigen-presenting polypeptide, and enucleation, thereby making an enucleated cell, thereby making the immunologically compatible aAPC. Methods of making an aAPC are described herein, however it is to be understood that these methods are non-limiting.

### Physical Characteristics of Engineered Erythroid Cells

In some embodiments, the erythroid cells described herein have one or more (e.g., 2, 3, 4, or more) physical characteristics described herein, e.g., osmotic fragility, cell size, hemoglobin concentration, or phosphatidylserine content. While not wishing to be bound by theory, in some embodiments an engineered erythroid cell that expresses an exogenous protein has physical characteristics that resemble a wild-type, untreated erythroid cell. In contrast, a hypotonically loaded erythroid cell sometimes displays aberrant physical characteristics such as increased osmotic fragility, altered cell size, reduced hemoglobin concentration, or increased phosphatidylserine levels on the outer leaflet of the cell membrane.

In some embodiments, the engineered erythroid cell, e.g. enucleated cell, comprises an exogenous protein that was encoded by an exogenous nucleic acid that was not retained by the cell, has not been purified, or has not existed fully outside an erythroid cell. In some embodiments, the erythroid cell is in a composition that lacks a stabilizer.

### Osmotic Fragility

In some embodiments, the engineered erythroid cell, e.g. enucleated cell, exhibits substantially the same osmotic membrane fragility as an isolated, uncultured erythroid cell that does not comprise an exogenous polypeptide. In some embodiments, the population of engineered erythroid cells has an osmotic fragility of less than 50% cell lysis at 0.3%, 0.35%, 0.4%, 0.45%, or 0.5% NaCl. Osmotic fragility can be assayed using the method of Example 59 of WO2015/073587, which is herein incorporated by reference in its entirety.

### Cell Size

In some embodiments, the engineered erythroid cell, e.g. enucleated cell, has approximately the diameter or volume as a wild-type, untreated erythroid cell. In some embodiments, the population of erythroid cells has an average diameter of about 4, 5, 6, 7, or 8 microns, and optionally the standard deviation of the population is less than 1, 2, or 3 microns. In some embodiments, the one or more erythroid cell has a diameter of about 4-8, 5-7, or about 6 microns. In some embodiments, the diameter of the erythroid cell is less than about 1 micron, larger than about 20 microns, between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns or between about 10 microns and about 30 microns. Cell diameter is measured, in some embodiments, using an Advia 120 hematology system.

In some embodiment the volume of the mean corpuscular volume of the erythroid cells is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In some embodiments the mean corpuscular volume of the erythroid cells is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In some embodiments the mean corpuscular volume of the erythroid cells is between 80-100, 100-200, 200-300, 300-400, or 400-500 femtoliters (fL). In some embodiments, a population of erythroid cells has a mean corpuscular volume set out in this paragraph and the standard deviation of the population is less than 50, 40, 30, 20, 10, 5, or 2 fL. The mean corpuscular volume is measured, in some embodiments, using a hematological analysis instrument, e.g., a Coulter counter.

Hemoglobin Concentration

In some embodiments, the engineered erythroid cell, e.g. enucleated cell, has a hemoglobin content similar to a wild-type, untreated erythroid cell. In some embodiments, the erythroid cells comprise greater than 1%, 2%, 3%, 4%, 5%, 6%, 7%, 8%, 9% or greater than 10% fetal hemoglobin. In some embodiments, the erythroid cells comprise at least about 20, 22, 24, 26, 28, or 30 pg, and optionally up to about 30 pg, of total hemoglobin. Hemoglobin levels are determined, in some embodiments, using the Drabkin's reagent method of Example 33 of WO2015/073587, which is herein incorporated by reference in its entirety.

Phosphatidylserine Content

In some embodiments, the engineered erythroid cell, e.g. artificial antigen presenting cells as described herein or the enucleated cell, has approximately the same phosphatidylserine content on the outer leaflet of its cell membrane as a wild-type, untreated erythroid cell. Phosphatidylserine is predominantly on the inner leaflet of the cell membrane of wild-type, untreated erythroid cells, and hypotonic loading can cause the phosphatidylserine to distribute to the outer leaflet where it can trigger an immune response. In some embodiments, the population of engineered erythroid cells (e.g. artificial antigen presenting cells as described herein) or enucleated cells comprises less than about 30, 25, 20, 15, 10, 9, 8, 6, 5, 4, 3, 2, or 1% of cells that are positive for Annexin V staining. Phosphatidylserine exposure is assessed, in some embodiments, by staining for Annexin-V-FITC, which binds preferentially to PS, and measuring FITC fluorescence by flow cytometry, e.g., using the method of Example 54 of WO2015/073587, which is herein incorporated by reference in its entirety.

Other Characteristics

In some embodiments, an engineered erythroid cell (e.g., engineered enucleated erythroid cell) or an engineered enucleated cell, or a population of engineered erythroid cells or engineered enucleated cells comprises one or more of (e.g., all of) endogenous GPA (C235a), transferrin receptor (CD71), Band 3 (CD233), or integrin alpha4 (C49d). These proteins can be measured, e.g., as described in Example 10 of International Application Publication No. WO2018/009838, which is herein incorporated by reference in its entirety. The percentage of GPA-positive cells and Band 3-positive cells typically increases during maturation of an erythroid cell, and the percentage of integrin alpha4-positive typically remains high throughout maturation.

In some embodiments, the population of erythroid cells comprises at least about 50%, 60%, 70%, 80%, 90%, or 95% (and optionally up to 90 or 100%) of cells that are positive for GPA. The presence of GPA is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% GPA⁺ (i.e., CD235a⁺) cells. In some embodiments, the population of engineered erythroid cells (engineered enucleated erythroid cells) or engineered enucleated cells comprises between about 50% and about 100% (e.g., from about 60% and about 100%, from about 65% and about 100%, from about 70% and about 100%, from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) GPA⁺ cells. The presence of GPA is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD71⁺ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD71⁺ cells. The presence of CD71 (transferrin receptor) is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD233⁺ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD233⁺ cells. The presence of CD233 (Band 3) is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD47⁺ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD47⁺ cells. The presence of CD47 (integrin associate protein) is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD36⁻ (CD36-negative) cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD36⁻ (CD36-negative) cells. The presence of CD36 is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD34⁻ (CD34-negative) cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD34⁻ (CD34-negative) cells. The presence of CD34 is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺ cells.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺/CD347CD36⁻ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺/CD347CD36⁻ cells.

In some embodiments, a population of engineered erythroid cells (engineered enucleated erythroid cells) or engineered enucleated cells comprising erythroid cells comprises less than about 10%, 9%, 8%, 7%, 6%, 5%, 4%, 3%, 2%, or 1% echinocytes.

In some embodiments, a population of engineered erythroid cells (e.g. artificial antigen presenting cells as described herein) comprising erythroid cells comprises less than about 10%, 9%, 8%, 7%, 6%, 5%, 4%, 3%, 2%, or 1% echinocytes.

In some embodiments, a population of engineered erythroid cells (engineered enucleated erythroid cells) or engineered enucleated cells comprises less than about 10%, 9%, 8%, 7%, 6%, 5%, 4%, 3%, 2%, or 1% pyrenocytes.

In some embodiments, an erythroid cell is enucleated, e.g., a population of cells comprising erythroid cells used as a therapeutic preparation described herein is greater than 50%, 60%, 70%, 80%, 90% enucleated. In some embodiments, a cell, e.g., an erythroid cell, contains a nucleus that is non-functional, e.g., has been inactivated. In some embodiments, the engineered erythroid cell is an enucleated cell. In some embodiments, the engineered erythroid cell is a nucleated cell.

### Isolating Erythrocytes

Mature erythrocytes may be isolated using various methods such as, for example, a cell washer, a continuous flow cell separator, density gradient separation, fluorescence-activated cell sorting (FACS), Miltenyi immunomagnetic depletion (MACS), or a combination of these methods (See, e.g., van der Berg et al., Clin. Chem. 33:1081-1082 (1987); Bar-Zvi et al., J. Biol. Chem. 262:17719-17723 (1987); Goodman et al., Exp. Biol. Med. 232:1470-1476 (2007)).

Erythrocytes may be isolated from whole blood by simple centrifugation (See, e.g., van der Berg et al., Clin. Chem. 33:1081-1082 (1987)). For example, EDTA-anticoagulated whole blood may be centrifuged at 800×g for 10 min at 4° C. The platelet-rich plasma and buffy coat are removed and the red blood cells are washed three times with isotonic saline solution (NaCl, 9 g/L).

Alternatively, erythrocytes may be isolated using density gradient centrifugation with various separation mediums such as, for example, Ficoll, Hypaque, Histopaque, Percoll, Sigmacell, or combinations thereof. For example, a volume of Histopaque-1077 is layered on top of an equal volume of Histopaque-1119. EDTA-anticoagulated whole blood diluted 1:1 in an equal volume of isotonic saline solution (NaCl, 9 g/L) is layered on top of the Histopaque and the sample is centrifuged at 700×g for 30 min at room temperature. Under these conditions, granulocytes migrate to the 1077/1119 interface, lymphocytes, other mononuclear cells and platelets remain at the plasma/1077 interface, and the red blood cells are pelleted. The red blood cells are washed twice with isotonic saline solution.

Alternatively, erythrocytes may be isolated by centrifugation using a Percoll step gradient (See, e.g., Bar-Zvi et al., J. Biol. Chem. 262:17719-17723 (1987)). For example, fresh blood is mixed with an anticoagulant solution containing 75 mM sodium citrate and 38 mM citric acid and the cells washed briefly in Hepes-buffered saline. Leukocytes and platelets are removed by adsorption with a mixture of α-cellulose and Sigmacell (1:1). The erythrocytes are further isolated from reticulocytes and residual white blood cells by centrifugation through a 45/75% Percoll step gradient for 10 min at 2500 rpm in a Sorvall SS34 rotor. The erythrocytes are recovered in the pellet while reticulocytes band at the 45/75% interface and the remaining white blood cells band at the 0/45% interface. The Percoll is removed from the erythrocytes by several washes in Hepes-buffered saline. Other materials that may be used to generate density gradients for isolation of erythrocytes include OPTIPREP, a 60% solution of iodixanol in water (from Axis-Shield, Dundee, Scotland).

Erythrocytes may be separated from reticulocytes, for example, using flow cytometry (See, e.g., Goodman et al., Exp. Biol. Med. 232:1470-1476 (2007)). In this instance, whole blood is centrifuged (550×g, 20 min, 25° C.) to separate cells from plasma. The cell pellet is resuspended in phosphate buffered saline solution and further fractionated on Ficoll-Paque (1.077 density), for example, by centrifugation (400×g, 30 min, 25° C.) to separate the erythrocytes from the white blood cells. The resulting cell pellet is resuspended in RPMI supplemented with 10% fetal bovine serum and sorted on a FACS instrument such as, for example, a Becton Dickinson FACSCalibur (BD Biosciences, Franklin Lakes, N.J., USA) based on size and granularity.

Erythrocytes may be isolated by immunomagnetic depletion (See, e.g., Goodman, et al., (2007) Exp. Biol. Med. 232:1470-1476). In this instance, magnetic beads with cell-type specific antibodies are used to eliminate non-erythrocytes. For example, erythrocytes are isolated from the majority of other blood components using a density gradient as described herein followed by immunomagnetic depletion of any residual reticulocytes. The cells are pre-treated with human antibody serum for 20 min at 25° C. and then treated with antibodies against reticulocyte specific antigens such as, for example, CD71 and CD36. The antibodies may be directly attached to magnetic beads or conjugated to PE, for example, to which magnetic beads with anti-PE antibody will react. The antibody-magnetic bead complex is able to selectively extract residual reticulocytes, for example, from the erythrocyte population.

Erythrocytes may also be isolated using apheresis. The process of apheresis involves removal of whole blood from a patient or donor, separation of blood components using centrifugation or cell sorting, withdrawal of one or more of the separated portions, and transfusion of remaining components back into the patient or donor. A number of instruments are currently in use for this purpose such as for example the Amicus and Alyx instruments from Baxter (Deerfield, Ill., USA), the Trima Accel instrument from Gambro BCT (Lakewood, Colo., USA), and the MCS+9000 instrument from Haemonetics (Braintree, Mass., USA). Additional purification methods may be necessary to achieve the appropriate degree of cell purity.

Reticulocytes are immature red blood cells and compose approximately 1% of the red blood cells in the human body. Reticulocytes develop and mature in the bone marrow. Once released into circulation, reticulocytes rapidly undergo terminal differentiation to mature erythrocytes. Like mature erythrocytes, reticulocytes do not have a cell nucleus. Unlike mature erythrocytes, reticulocytes maintain the ability to perform protein synthesis. In some embodiments, the aAPC comprises an enucleated erythrocyte.

Reticulocytes of varying age may be isolated from peripheral blood based on the differences in cell density as the reticulocytes mature. Reticulocytes may be isolated from peripheral blood using differential centrifugation through various density gradients. For example, Percoll gradients may be used to isolate reticulocytes (See, e.g., Noble et al., Blood 74:475-481 (1989)). Sterile isotonic Percoll solutions of density 1.096 and 1.058 g/ml are made by diluting Percoll (Sigma-Aldrich, Saint Louis, Mo., USA) to a final concentration of 10 mM triethanolamine, 117 mM NaCl, 5 mM glucose, and 1.5 mg/ml bovine serum albumin (BSA). These solutions have an osmolarity between 295 and 310 mOsm. Five milliliters, for example, of the first Percoll solution (density 1.096) is added to a sterile 15 ml conical centrifuge tube. Two milliliters, for example, of the second Percoll solution (density 1.058) is layered over the higher density first Percoll solution. Two to four milliliters of whole blood are layered on top of the tube. The tube is centrifuged at 250×g for 30 min in a refrigerated centrifuge with swing-out tube holders. Reticulocytes and some white cells migrate to the interface between the two Percoll layers. The cells at the interface are transferred to a new tube and washed twice with phosphate buffered saline (PBS) with 5 mM glucose, 0.03 mM sodium azide and 1 mg/ml BSA. Residual white blood cells are removed by chromatography in PBS over a size exclusion column.

Alternatively, reticulocytes may be isolated by positive selection using an immunomagnetic separation approach (See, e.g., Brun et al., Blood 76:2397-2403 (1990)). This approach takes advantage of the large number of transferrin receptors that are expressed on the surface of reticulocytes relative to erythrocytes prior to maturation. Magnetic beads coated with an antibody to the transferrin receptor may be used to selectively isolate reticulocytes from a mixed blood cell population. Antibodies to the transferrin receptor of a variety of mammalian species, including human, are available from commercial sources (e.g., Affinity BioReagents, Golden, Colo., USA; Sigma-Aldrich, Saint Louis, Mo., USA). The transferrin antibody may be directly linked to the magnetic beads. Alternatively, the transferrin antibody may be indirectly linked to the magnetic beads via a secondary antibody. For example, mouse monoclonal antibody 10D2 (Affinity BioReagents, Golden, Colo., USA) against human transferrin may be mixed with immunomagnetic beads coated with a sheep anti-mouse immunoglobulin G (Dynal/Invitrogen, Carlsbad, Calif., USA). The immunomagnetic beads are then incubated with a leukocyte-depleted red blood cell fraction. The beads and red blood cells are incubated at 22° C. with gentle mixing for 60-90 min followed by isolation of the beads with attached reticulocytes using a magnetic field. The isolated reticulocytes may be removed from the magnetic beads using, for example, DETACHaBEAD solution (from Invitrogen, Carlsbad, Calif., USA). Alternatively, reticulocytes may be isolated from in vitro growth and maturation of CD34+ hematopoietic stem cells using the methods described herein.

Terminally-differentiated, enucleated erythrocytes can be separated from other cells based on their DNA content. In a non-limiting example, cells are first labeled with a vital DNA dye, such as Hoechst 33342 (Invitrogen Corp.). Hoechst 33342 is a cell-permeant nuclear counterstain that emits blue fluorescence when bound to double-stranded DNA. Undifferentiated precursor cells, macrophages or other nucleated cells in the culture are stained by Hoechst 33342, while enucleated erythrocytes are Hoechst-negative. The Hoechst-positive cells can be separated from enucleated erythrocytes by using fluorescence activated cell sorters or other cell sorting techniques. The Hoechst dye can be removed from the isolated erythrocytes by dialysis or other suitable methods.

### Vehicles for Polypeptides Described Herein

While in many embodiments herein, the one or more (e.g., two or more) exogenous polypeptides are situated on or in an enucleated erythroid cell, it is understood that any polypeptide or combination of exogenous polypeptides described herein can also be situated on or in another vehicle. The vehicle can comprise, e.g., a cell, an erythroid cell, a corpuscle, a nanoparticle, a micelle, a liposome, or an exosome. For instance, in some aspects, the present disclosure provides a vehicle (e.g., a cell, an erythroid cell, a corpuscle, a nanoparticle, a micelle, a liposome, or an exosome) comprising, e.g., on its surface, one or more agents described herein. In some embodiments, the one or more agents comprise an agent selected from a polypeptide of any of Tables 1 or 14-24, or a fragment or variant thereof, or an antibody molecule thereto. In some embodiments, the vehicle comprises two or more agents described herein, e.g., any pair of agents described herein.

### Heterogeneous Populations of Cells

While in many embodiments herein, the one or more (e.g., two or more) exogenous polypeptides are situated on or in a single cell, it is understood that any polypeptide or combination of polypeptides described herein can also be situated on a plurality of cells. For instance, in some aspects, the disclosure provides a plurality of erythroid cells, wherein a first cell of the plurality comprises a first exogenous polypeptide and a second cell of the plurality comprises a second exogenous polypeptide. In some embodiments, the plurality of cells comprises two or more polypeptides described herein, e.g., any pair of polypeptides described herein. In some embodiments, less than 90%, 80%, 70%, 60%, 50%, 40%, 30%, 20%, 10%, 5%, 2%, or 1% of the cells in the population comprise both the first exogenous polypeptide and the second exogenous polypeptide.

### Cells Encapsulated in a Membrane

In some embodiments, enucleated erythroid cells or other vehicles described herein are encapsulated in a membrane, e.g., semi-permeable membrane. In some embodiments, the membrane comprises a polysaccharide, e.g., an anionic polysaccharide alginate. In some embodiments, the semipermeable membrane does not allow cells to pass through, but allows passage of small molecules or macromolecules, e.g., metabolites, proteins, or DNA. In some embodiments, the membrane is one described in Lienert et al., “Synthetic biology in mammalian cells: next generation research tools and therapeutics” Nature Reviews Molecular Cell Biology 15, 95-107 (2014), incorporated herein by reference in its entirety. While not wishing to be bound by theory, in some embodiments, the membrane shields the cells from the immune system and/or keeps a plurality of cells in proximity, facilitating interaction with each other or each other's products.

### Erythrocyte Precursor Cells

Provided herein are engineered erythrocyte precursor cells, and methods of making the engineered erythrocyte precursor cells, reticulocytes and erythrocytes.

Pluripotent stem cells give rise to erythrocytes by the process of erythropoiesis. The stem cell looks like a small lymphocyte and lacks the functional capabilities of the erythrocyte. The stem cells have the capacity of infinite division, something the mature cells lack. Some of the daughter cells arising from the stem cell acquire erythroid characters over generations and time. Most of the erythroid cells in the bone marrow have a distinct morphology but commitment to erythroid maturation is seen even in cells that have not acquired morphological features distinctive of the erythroid lineage. These cells are recognized by the type of colonies they form in vitro. Two such cells are recognized. Burst-forming unit erythroid (BFU-E) arise from the stem cell and gives rise to colony-forming unit erythroid (CFU-E). CFU-E gives rise to pronormoblast, the most immature of erythroid cells with a distinct morphology. BFU-E and CFU-E form a very small fraction of bone marrow cells. Morphologically five erythroid precursors are identifiable in the bone marrow stained with Romanovsky stains. The five stages from the most immature to the most mature are the proerythroblast, the basophilic normoblast (early erythroblast), polychromatophilic normoblast (intermediate erythroblast), orthochromatophilic normoblast (late erythroblast) and reticulocyte. BFU-E (burst forming unit-erythroid), CFU-E (erythroid colony-forming unit), pronormoblast (proerythroblast), basophilic normoblast, polychromatophilic normoblast and orthochromatophilic normoblast are lineage restricted.

Table 13 below summarizes the morphological features of erythrocyte precursor and erythrocyte cells.

TABLE 13
Morphological features of erythrocyte precursor and erythrocyte cells

| Cell | Nucleus |
|---|---|
| Hematopoietic stem cell (HSC) | Yes |
| CMP (Common myeloid progenitor) | Yes |
| CFU-S (spleen colony forming cell; | Yes; Can differentiate into |
| myeloid precursor cell) | erythrocytes, platelets, |
| | macrophages. |
| BFU-E (burst forming unit-erythroid) | Yes |
| CFU-E (erythroid colony-forming | Yes |
| unit) | |
| Pronormoblast (proerythroblast) | Yes; fine chromatin, many nucleoli |
| Basophilic Normoblast | Yes; granular chromatin, no |
| | nucleoli |
| Polychromatophilic Normoblast | Yes; chromatin is visibly clumped |
| | with dark staining areas |
| Orthochromatophilic normoblast | Yes; featureless nucleus with dense |
| | chromatin |
| Reticulocyte | No Nucleus |
| Erythrocyte (fully matured RBC) | No Nucleus |

Normal human erythrocytes express CD36, an adhesion molecule of monocytes, platelets, and endothelial cells (van Schravendijk M R et al., Blood. 1992 Oct. 15; 80(8):2105-14). Accordingly, in some embodiments, an anti-CD36 antibody can be used to identify human erythrocytes.

Any type of cell known in the art that is capable of differentiating into an erythrocyte, i.e., any erythrocyte precursor cell, can be modified in accordance with the methods described herein to produce engineered erythrocyte precursor cells. In certain embodiments, the erythrocyte precursor cells modified in accordance with the methods described herein are cells that are in the process of differentiating into an erythrocyte, i.e., the cells are of a type known to exist during mammalian erythropoiesis. For example, the cells may be pluripotent hematopoietic stem cells (HSCs) or CD34+ cells, multipotent myeloid progenitor cells, CFU-S cells, BFU-E cells, CFU-E cells, pronormoblasts (proerythroblast), basophilic normoblasts, polychromatophilic normoblasts and orthochromatophilic normoblasts. The modified erythrocyte precursor cells provided herein can be differentiated into engineered reticulocytes or erythrocytes in vitro using methods known in the art, i.e., using molecules known to promote erythropoiesis, e.g., SCF, Erythropoietin, IL-3, and/or GM-CSF, described herein below. Alternatively, the modified erythrocyte precursor cells are provided in a composition of the invention, and are capable of differentiating into erythrocytes upon administration to a subject in vivo.

In some embodiments, the erythroid precursor cell has not been genetically modified to delete and/or alter expression of an endogenous antigen presenting polypeptide (e.g. a MHC class I or MHC class II molecule).

In some embodiments, the erythrocyte precursor cells, e.g., hematopoietic stem cells, are from an O-negative donor.

### Culturing

Sources for generating aAPCs described herein include circulating cells such as erythroid cells. A suitable cell source may be isolated from a subject as described herein from patient-derived hematopoietic or erythroid progenitor cells, derived from immortalized erythroid cell lines, or derived from induced pluripotent stem cells, optionally cultured and differentiated. Methods for generating erythrocytes using cell culture techniques are well known in the art, e.g., Giarratana et al., Blood 2011, 118:5071, Huang et al., Mol Ther 2013, epub ahead of print September 3, or Kurita et al., PLOS One 2013, 8:e59890, the contents of each of which is incorporated by reference in its entirety herein. Protocols vary according to growth factors, starting cell lines, culture period, and morphological traits by which the resulting cells are characterized. Culture systems have also been established for blood production that may substitute for donor transfusions (Fibach et al. 1989 Blood 73:100). Recently, CD34+ cells were differentiated to the reticulocyte stage, followed by successful transfusion into a human subject (Giarratana et al., Blood 2011, 118:5071). It will be understood that in some embodiments, the patient-derived hematopoietic or erythroid progenitor cells, e.g., hematopoetic stem cells, are from an O-negative donor.

Provided herein are culturing methods for erythroid cells and aAPCs derived from erythroid cells. Erythroid cells can be cultured from hematopoietic progenitor cells, including, for example, CD34+ hematopoietic progenitor cells (Giarratana et al., Blood 2011, 118:5071), induced pluripotent stem cells (Kurita et al., PLOS One 2013, 8:e59890), and embryonic stem cells (Hirose et al. 2013 Stem Cell Reports 1:499). Cocktails of growth and differentiation factors that are suitable to expand and differentiate progenitor cells are known in the art. Examples of suitable expansion and differentiation factors include, but are not limited to, stem cell factor (SCF), an interleukin (IL) such as IL-1, IL-2, IL-3, IL-4, IL-5, IL-6, IL-7, IL-8, IL-9, IL-11, IL-12, CSF, G-CSF, thrombopoietin (TPO), GM-CSF, erythropoietin (EPO), Flt3, Flt2, PIXY 321, and leukemia inhibitory factor (LIF).

Erythroid cells can be cultured from hematopoietic progenitors, such as CD34+ cells, by contacting the progenitor cells with defined factors in a multi-step culture process. For example, erythroid cells can be cultured from hematopoietic progenitors in a three-step process.

The first step may comprise contacting the cells in culture with stem cell factor (SCF) at 1-1000 ng/mL, erythropoietin (EPO) at 1-100 U/mL, and interleukin-3 (IL-3) at 0.1-100 ng/mL. The first step optionally comprises contacting the cells in culture with a ligand that binds and activates a nuclear hormone receptor, such as e.g., the glucocorticoid receptor, the estrogen receptor, the progesterone receptor, the androgen receptor, or the pregnane x receptor. The ligands for these receptors include, for example, a corticosteroid, such as, e.g., dexamethasone at 10 nM-100 μM or hydrocortisone at 10 nM-100 μM; an estrogen, such as, e.g., beta-estradiol at 10 nM-100 μM; a progestogen, such as, e.g., progesterone at 10 nM-100 μM, hydroxyprogesterone at 10 nM-100 μM, 5a-dihydroprogesterone at 10 nM-100 μM, 11-deoxycorticosterone at 10 nM-100 μM, or a synthetic progestin, such as, e.g., chlormadinone acetate at 10 nM-100 μM; an androgen, such as, e.g., testosterone at 10 nM-100 μM, dihydrotestosterone at 10 nM-100 μM or androstenedione at 10 nM-100 μM; or a pregnane x receptor ligand, such as, e.g., rifampicin at 10 nM-100 μM, hyperforin at 10 nM-100 St. John's Wort (hypericin) at 10 nM-100 μM, or vitamin E-like molecules, such as, e.g., tocopherol at 10 nM-100 The first step may also optionally comprise contacting the cells in culture with an insulin-like molecule, such as, e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The first step further may optionally comprise contacting the cells in culture with transferrin at 0.1-5 mg/mL.

The first step may optionally comprise contacting the cells in culture with one or more interleukins (IL) or growth factors such as, e.g., IL-1, IL-2, IL-4, IL-5, IL-6, IL-7, IL-8, IL-9, IL-11, IL-12, granulocyte colony-stimulating factor (G-CSF), macrophage colony-stimulating factor (M-CSF), granulocyte-macrophage colony-stimulating factor (GM-CSF), thrombopoietin, fibroblast growth factor (FGF), platelet-derived growth factor (PDGF), transforming growth factor beta (TGF-B), tumor necrosis factor alpha (TNF-A), megakaryocyte growth and development factor (MGDF), leukemia inhibitory factor (LIF), and Flt3 ligand. Each interleukin or growth factor may typically be supplied at a concentration of 0.1-100 ng/mL. The first step may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

The second step may comprise contacting the cells in culture with stem cell factor (SCF) at 1-1000 ng/mL and erythropoietin (EPO) at 1-100 U/mL. The second step may also optionally comprise contacting the cells in culture with an insulin-like molecule, such as e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The second step may further optionally comprise contacting the cells in culture with transferrin at 0.1-5 mg/mL. The second may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

The third step may comprise contacting the cells in culture with erythropoietin (EPO) at 1-100 U/mL. The third step may optionally comprise contacting the cells in culture with stem cell factor (SCF) at 1-1000 ng/mL. The third step may further optionally comprise contacting the cells in culture with an insulin-like molecule, such as e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The third step may also optionally comprise contacting the cells in culture with transferrin at 0.1-5 mg/mL. The third step may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

In some embodiments, methods of expansion and differentiation of the aAPCs comprising an erythroid cell, e.g. an enucleated cell, presenting (e.g. comprising on the cell surface) one or more exogenous polypeptides, do not include culturing the aAPCs in a medium comprising a myeloproliferative receptor (mpl) ligand.

The culture process may optionally comprise contacting cells by a method known in the art with a molecule, e.g., a DNA molecule, an RNA molecule, a mRNA, an siRNA, a microRNA, a lncRNA, a shRNA, a hormone, or a small molecule, that activates or knocks down one or more genes. Target genes can include, for example, genes that encode a transcription factor, a growth factor, or a growth factor receptor, including but not limited to, e.g., GATA1, GATA2, CMyc, hTERT, p53, EPO, SCF, insulin, EPO-R, SCF-R, transferrin-R, insulin-R.

In some embodiments, CD34+ cells are placed in a culture containing varying amounts of IMDM, FBS, glutamine, BSA, holotransferrin, insulin, dexamethasone, .beta.-estradiol, IL-3, SCF, and erythropoietin, in three separate differentiation stages for a total of 22 days.

In some embodiments, CD34+ cells are placed in a culture containing varying amounts of IMDM, FBS, glutamine, BSA, holotransferrin, insulin, dexamethasone, .beta.-estradiol, IL-3, SCF, and thrombopoietin, in three separate differentiation stages for a total of 14 days.

In some embodiments, CD34+ cells are placed in a culture containing varying amounts of IMDM, FBS, glutamine, BSA, holotransferrin, insulin, dexamethasone, .beta.-estradiol, IL-3, SCF, and GCSF, in three separate differentiation stages for a total of 15 days.

In some embodiments, the erythroid cells are expanded at least 100, 1000, 2000, 5000, 10,000, 20,000, 50,000, or 100,000 fold (and optionally up to 100,000, 200,000, or 500,000 fold). Number of cells is measured, in some embodiments, using an automated cell counter. In some embodiments, the population of erythroid cells comprises at least 30, 35, 40, 45, 50, 55, 60, 65, 70, 75, 80, 85, 90, 95, or 98% (and optionally up to about 80, 90, or 100%) engineered erythroid cells. Enucleation is measured, in some embodiments, by FACS using a nuclear stain. In some embodiments, at least 30, 35, 40, 45, 50, 55, 60, 65, 70, 75, or 80% (and optionally up to about 70, 80, 90, or 100%) of erythroid cells in the population comprise one or more (e.g., 2, 3, 4 or more) of the exogenous polypeptides. Expression of the polypeptides is measured, in some embodiments, by erythroid cells using labeled antibodies against the polypeptides. In some embodiments, at least 30, 35, 40, 45, 50, 55, 60, 65, 70, 75, or 80% (and optionally up to about 70, 80, 90, or 100%) of erythroid cells in the population are enucleated and comprise one or more (e.g., 2, 3, 4, or more) of the exogenous polypeptides. In some embodiments, the population of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10″-5×10¹¹, 5×10¹¹-1×10¹², 1×10¹²-2×10¹², 2×10¹²-5×10¹², or 5×10¹²-1×10¹³ cells.

In some embodiments, it may be desirable during culturing to only partially differentiate the erythroid progenitor cells, e.g., hematopoietic stem cells, in vitro, allowing further differentiation, e.g., differentiation into reticulocytes or fully mature erythrocytes, to occur upon introduction to a subject in vivo (See, e.g., Neildez-Nguyen et al., Nature Biotech. 20:467-472 (2002)). It will be understood that, in various embodiments of the invention, maturation and/or differentiation in vitro may be arrested at any stage desired. For example, isolated CD34+ hematopoietic stem cells may be expanded in vitro as described elsewhere herein, e.g., in medium containing various factors, including, for example, interleukin 3, Flt3 ligand, stem cell factor, thrombopoietin, erythropoietin, transferrin, and insulin growth factor, to reach a desired stage of differentiation. The resulting engineered erythroid cells may be characterized by the surface expression of CD36 and GPA, and other characteristics specific to the particular desired cell type, and may be transfused into a subject where terminal differentiation to mature erythrocytes is allowed to occur.

In some embodiments, engineered erythroid cells are partially expanded from erythroid progenitor cells to any stage of maturation prior to but not including enucleation, and thus remain nucleated cells, e.g., erythrocyte precursor cells. In certain embodiments, the resulting cells are nucleated and erythroid lineage restricted. In certain embodiments, the resulting cells are selected from multipotent myeloid progenitor cells, CFU-S cells, BFU-E cells, CFU-E cells, pronormoblasts (proerythroblast), basophilic normoblasts, polychromatophilic normoblasts and orthochromatophilic normoblasts. The final differentiation steps, including enucleation, occur only after administration of the engineered erythroid cell to a subject, that is, in such embodiments, the enucleation step occurs in vivo. In another embodiment, engineered erythroid cells are expanded and differentiated in vitro through the stage of enucleation to become, e.g., reticulocytes. In such embodiments where the engineered erythroid cells are differentiated to the stage of reticulocytes, the final differentiation step to become erythrocytes occurs only after administration of the engineered erythroid cell to a subject, that is, the terminal differentiation step occurs in vivo. In another embodiment, engineered erythroid cells are expanded and differentiated in vitro through the terminal differentiation stage to become erythrocytes.

It will be further recognized that in some embodiments, the engineered erythroid cells may be expanded and differentiated from erythroid progenitor cells, e.g., hematopoietic stem cells, to become hematopoietic cells of different lineage, such as, for example, to become platelets. Methods for maturing and differentiating hematopoietic cells of various lineages, such as platelets, are well known in the art to the skilled artisan. In some embodiments, such engineered platelets expressing exogenous polypeptides as described herein are considered to be encompassed by the present invention.

In some embodiments of the above aspects and embodiments, the engineered erythroid cell is an enucleated cell. In some embodiments of the above aspects and embodiments, the engineered erythroid cell is a nucleated cell.

In some embodiments, an enucleated cell provided herein is a platelet. Methods of manufacturing platelets in vitro are known in the art (see, e.g., Wang and Zheng (2016) Springerplus 5(1): 787, and U.S. Pat. No. 9,574,178). Methods of manufacturing platelets including an exogenous polypeptide are described, e.g., in International Patent Application Publication Nos. WO2015/073587 and WO2015/153102, each of which is incorporated by reference in its entirety. Platelet production is in part regulated by signaling mechanisms induced by interaction between thrombopoietin (TPO) and its cellular receptor TPOR/MPUc-MPL. In addition, multiple cytokines (e.g., stem cell factor (SCF), IL-1, IL-3, IL-6, IL-11, leukemia inhibiting factor (LIF), G-CSF, GM-CSF, M-CSF, erythropoietin (EPO), kit ligand, and interferon) have been shown to possess thrombocytopoietic activity.

In some embodiments, platelets are generated from hematopoietic progenitor cells, such as CD34⁺ hematopoietic stem cells, induced pluripotent stem cells or embryonic stem cells. In some embodiments, platelets are produced by contacting the progenitor cells with defined factors in a multi-step culture process. In some embodiments, the multi-step culture process comprises: culturing a population of hematopoietic progenitor cells under conditions suitable to produce a population of megakaryocyte progenitor cells, and culturing the population of megakaryocyte progenitor cells under conditions suitable to produce platelets. Cocktails of growth and differentiation factors that are suitable to expand and differentiate progenitor cells and produce platelets are known in the art. Examples of suitable expansion and differentiation factors include, but are not limited to, stem cell factor (SCF), Flt-3/Flk-2 ligand (FL), TPO, IL-11, IL-3, IL-6, and IL-9. For instance, in some embodiments, platelets may be produced by seeding CD34⁺ HSCs in a serum-free medium at 2-4×10⁴ cells/mL, and refreshing the medium on culture day 4 by adding an equal volume of media. On culture day 6, cells are counted and analyzed: 1.5×10⁵ cells are washed and placed in 1 mL of the same medium supplemented with a cytokine cocktail comprising TPO (30 ng/mL), SCF (1 ng/mL), IL-6 (7.5 ng/mL), and IL-9 (13.5 ng/mL) to induce megakaryocyte differentiation. At culture day 10, from about one quarter to about half of the suspension culture is replaced with fresh media. The cells are cultured in a humidified atmosphere (10% CO₂) at 39° C. for the first 6 culture days, and at 37° C. for the last 8 culture days. Viable nucleated cells are counted with a hemocytometer following trypan blue staining. The differentiation state of platelets in culture can be assessed by flow cytometry or quantitative PCR as described in Examples 44 and 45 of in International Patent Application Publication No. WO2015/073587, incorporated herein by reference.

### Expression of Exogenous Polypeptides

In some embodiments, the aAPC comprising an engineered erythroid cell or enucleated cell presenting one or more exogenous polypeptides is generated by contacting a suitable isolated cell, e.g., an erythroid cell, a reticulocyte, an erythroid cell precursor, a platelet, or a platelet precursor, with an exogenous nucleic acid encoding a polypeptide of the disclosure (e.g. exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides).

In some embodiments, the exogenous polypeptide is encoded by a DNA, which is contacted with a nucleated erythroid precursor cell or a nucleated platelet precursor cell. In some embodiments, the exogenous polypeptide is encoded by an RNA, which is contacted with a platelet, a nucleate erythroid cell, a nucleated platelet precursor cell, or a reticulocyte. In some embodiments, the exogenous polypeptide is contacted with a primary platelet, a nucleated erythroid cell, a nucleated platelet precursor cell, a reticulocyte, or an erythrocyte.

An exogenous polypeptide may be expressed from a transgene introduced into an erythroid cell by electroporation, chemical or polymeric transfection, viral transduction, mechanical membrane disruption, or other method; an exogenous polypeptide that is expressed from mRNA that is introduced into a cell by electroporation, chemical or polymeric transfection, viral transduction, mechanical membrane disruption, or other method; an exogenous polypeptide that is over-expressed from the native locus by the introduction of an external factor, e.g., a transcriptional activator, transcriptional repressor, or secretory pathway enhancer; and/or a polypeptide that is synthesized, extracted, or produced from a production cell or other external system and incorporated into the erythroid cell.

Exogenous polypeptides (e.g. exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) can be introduced by transfection of single or multiple copies of genes, transduction with a virus, or electroporation in the presence of DNA or RNA. Methods for expression of exogenous proteins in mammalian cells are well known in the art. For example, expression of exogenous factor IX in hematopoietic cells is induced by viral transduction of CD34+ progenitor cells, see Chang et al., Nat Biotechnol 2006, 24:1017.

In some embodiments, the two or more polypeptides are encoded in a single nucleic acid, e.g. a single vector. In embodiments, the single vector has a separate promoter for each gene, has two proteins that are initially transcribed into a single polypeptide having a protease cleavage site in the middle, so that subsequent proteolytic processing yields two proteins, or any other suitable configuration. In some embodiments, the two or more polypeptides are encoded in two or more nucleic acids, e.g., each vector encodes one of the polypeptides.

Nucleic acids such as DNA expression vectors or mRNA for producing the exogenous polypeptides may be introduced into progenitor cells (e.g., an erythroid cell progenitor or a platelet progenitor and the like) that are suitable to produce the exogenous polypeptides described herein. The progenitor cells can be isolated from an original source or obtained from expanded progenitor cell population via routine recombinant technology as provided herein. In some instances, the expression vectors can be designed such that they can incorporate into the genome of cells by homologous or non-homologous recombination by methods known in the art.

In some embodiments, hematopoietic progenitor cells, e.g., CD34+ hematopoietic progenitor cells, are contacted with a nucleic acid or nucleic acids encoding one or more exogenous polypeptides, and the cells are allowed to expand and differentiate in culture.

In some instances, e.g., for an aAPC that is an erythroid cell comprising one or more exogenous polypeptides (e.g. exogenous antigenic polypeptides, exogenous antigen-presenting polypeptides, exogenous costimulatory polypeptides, exogenous T cell expansion polypeptides, exogenous co-inhibitory polypeptides, exogenous proliferation inhibitors, exogenous amino acid-depleting polypeptides, exogenous regulatory T cell expansion polypeptides, exogenous placeholder polypeptides), a nucleic acid encoding a polypeptide that can selectively target and cut the genome, for example a CRISPR/Cas9, transcriptional activator-like effector nuclease (TALEN), or zinc finger nuclease, is used to direct the insertion of the exogenous nucleic acid of the expression vector encoding the exogenous polypeptide to a particular genomic location, for example the CR1 locus (1q32.2), the hemoglobin locus (11p15.4). Thus, in one aspect, the present disclosure features a method of making an immunologically compatible artificial antigen presenting cell (aAPC), wherein the aAPC comprises an engineered erythroid cell that expresses an exogenous antigenic polypeptide, the method comprising contacting the aAPC with a nuclease and at least one gRNA which cleaves an endogenous MHC nucleic acid, wherein the endogenous MHC nucleic acid is repaired by a gene editing pathway and results in a decrease in the level of expression of the endogenous MHC nucleic acid, thereby making the immunologically compatible aAPC.

In some embodiments, one or more exogenous polypeptides (e.g. exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) may be cloned into plasmid constructs for transfection. Methods for transferring expression vectors into cells that are suitable to produce the aAPCs described herein include, but are not limited to, viral mediated gene transfer, liposome mediated transfer, transformation, gene guns, transfection and transduction, e.g., viral mediated gene transfer such as the use of vectors based on DNA viruses such as adenovirus, adenoassociated virus and herpes virus, as well as retroviral based vectors. Examples of modes of gene transfer include e.g., naked DNA, CaPO₄ precipitation, DEAE dextran, electroporation, protoplast fusion, lipofection, and cell microinjection.

In some embodiments, recombinant DNA encoding each exogenous polypeptide may be cloned into a lentiviral vector plasmid for integration into erythroid cells. In some embodiments, the lentiviral vector comprises DNA encoding a single exogenous polypeptide for integration into erythroid cells. In other embodiments, the lentiviral vector comprises two, three, four or more exogenous polypeptides as described herein for integration into erythroid cells. In some embodiments, recombinant DNA encoding the one or more exogenous polypeptides may be cloned into a plasmid DNA construct encoding a selectable trait, such as an antibiotic resistance gene. In some embodiments, recombinant DNA encoding the exogenous polypeptides may be cloned into a plasmid construct that is adapted to stably express each recombinant protein in the erythroid cells.

In some embodiments, the lentiviral system may be employed where the transfer vector with exogenous polypeptides sequences (e.g., one, two, three, four or more exogenous polypeptide sequences), an envelope vector, and a packaging vector are each transfected into host cells for virus production. In some embodiments, the lentiviral vectors may be transfected into host cells by any of calcium phosphate precipitation transfection, lipid based transfection, or electroporation, and incubated overnight. For embodiments where the exogenous polypeptide sequence may be accompanied by a fluorescence reporter, inspection of the host cells for florescence may be checked after overnight incubation. The culture medium of the host cells comprising virus particles may be harvested 2 or 3 times every 8-12 hours and centrifuged to sediment detached cells and debris. The culture medium may then be used directly, frozen or concentrated as needed.

A progenitor cell subject to transfer of an exogenous nucleic acid that encodes an exogenous polypeptide can be cultured under suitable conditions allowing for differentiation into mature enucleated red blood cells, e.g., the in vitro culturing process described herein. The resulting enucleated red blood cells display proteins associated with mature erythrocytes, e.g., hemoglobin, glycophorin A, and exogenous polypeptides which can be validated and quantified by standard methods (e.g., Western blotting or FACS analysis). Isolated mature enucleated red blood cells comprising an exogenous antigen-presenting polypeptide and an exogenous antigenic polypeptide and platelets comprising an exogenous antigen-presenting polypeptide and an exogenous antigenic polypeptide are two non-limiting examples of aAPCs of the disclosure.

In some embodiments, the aAPC is generated by contacting a reticulocyte with an exogenous nucleic acid encoding an antigenic polypeptide. In some embodiments, the antigenic polypeptide is encoded by an RNA which is contacted with a reticulocyte. In some embodiments, the antigenic polypeptide is a polypeptide which is contacted with a reticulocyte.

Isolated reticulocytes may be transfected with mRNA encoding one or more exogenous polypeptides (e.g. exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) to generate an aAPC. Messenger RNA may be derived from in vitro transcription of a cDNA plasmid construct containing the coding sequence corresponding to the one or more exogenous polypeptides. For example, the cDNA sequence corresponding to the exogenous polypeptide may be inserted into a cloning vector containing a promoter sequence compatible with specific RNA polymerases. For example, the cloning vector ZAP EXPRESS pBK-CMV (Stratagene, La Jolla, Calif., USA) contains T3 and T7 promoter sequence compatible with T3 and T7 RNA polymerase, respectively. For in vitro transcription of sense mRNA, the plasmid is linearized at a restriction site downstream of the stop codon(s) corresponding to the end of the coding sequence of the exogenous polypeptide. The mRNA is transcribed from the linear DNA template using a commercially available kit such as, for example, the RNAMAXX High Yield Transcription Kit (from Stratagene, La Jolla, Calif., USA). In some instances, it may be desirable to generate 5′-m7GpppG-capped mRNA. As such, transcription of a linearized cDNA template may be carried out using, for example, the mMESSAGE mMACHINE High Yield Capped RNA Transcription Kit from Ambion (Austin, Tex., USA). Transcription may be carried out in a reaction volume of 20-100 μl at 37° C. for 30 min to 4 h. The transcribed mRNA is purified from the reaction mix by a brief treatment with DNase I to eliminate the linearized DNA template followed by precipitation in 70% ethanol in the presence of lithium chloride, sodium acetate or ammonium acetate. The integrity of the transcribed mRNA may be assessed using electrophoresis with an agarose-formaldehyde gel or commercially available Novex pre-cast TBE gels (e.g., Novex, Invitrogen, Carlsbad, Calif., USA).

Messenger RNA encoding the one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) may be introduced into reticulocytes using a variety of approaches including, for example, lipofection and electroporation (van Tandeloo et al., Blood 98:49-56 (2001)). For lipofection, for example, 5 μg of in vitro transcribed mRNA in Opti-MEM (Invitrogen, Carlsbad, Calif., USA) is incubated for 5-15 min at a 1:4 ratio with the cationic lipid DMRIE-C(Invitrogen). Alternatively, a variety of other cationic lipids or cationic polymers may be used to transfect cells with mRNA including, for example, DOTAP, various forms of polyethylenimine, and polyL-lysine (Sigma-Aldrich, Saint Louis, Mo., USA), and Superfect (Qiagen, Inc., Valencia, Calif., USA; See, e.g., Bettinger et al., Nucleic Acids Res. 29:3882-3891 (2001)). The resulting mRNA/lipid complexes are incubated with cells (1-2×10⁶ cells/ml) for 2 h at 37° C., washed and returned to culture. For electroporation, for example, about 5 to 20×10⁶ cells in 500 μl of Opti-MEM (Invitrogen, Carlsbad, Calif., USA) are mixed with about 20 μg of in vitro transcribed mRNA and electroporated in a 0.4-cm cuvette using, for example, and Easyject Plus device (EquiBio, Kent, United Kingdom). In some instances, it may be necessary to test various voltages, capacitances and electroporation volumes to determine the useful conditions for transfection of a particular mRNA into a reticulocyte. In general, the electroporation parameters required to efficiently transfect cells with mRNA appear to be less detrimental to cells than those required for electroporation of DNA (van Tandeloo et al., Blood 98:49-56 (2001)).

Alternatively, mRNA may be transfected into a reticulocyte using a peptide-mediated RNA delivery strategy (see, e.g., Bettinger et al., Nucleic Acids Res. 29:3882-3891 (2001)). For example, the cationic lipid polyethylenimine 2 kDA (Sigma-Aldrich, Saint Louis, Mo., USA) may be combined with the melittin peptide (Alta Biosciences, Birmingham, UK) to increase the efficiency of mRNA transfection, particularly in post-mitotic primary cells. The mellitin peptide may be conjugated to the PEI using a disulfide cross-linker such as, for example, the heterobifunctional cross-linker succinimidyl 3-(2-pyridyldithio) propionate. In vitro transcribed mRNA is preincubated for 5 to 15 min with the mellitin-PEI to form an RNA/peptide/lipid complex. This complex is then added to cells in serum-free culture medium for 2 to 4 h at 37° C. in a 5% CO₂ humidified environment and then removed and the transfected cells allowed to continue growing in culture.

In some embodiments, the aAPC is generated by contacting a suitable isolated erythroid cell precursor or a platelet precursor with an exogenous nucleic acid encoding one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides). In some embodiments, the exogenous polypeptide is encoded by a DNA, which is contacted with a nucleated erythroid precursor cell or a nucleated platelet precursor cell. In some embodiments, the exogenous polypeptide is encoded by an RNA, which is contacted with a platelet, a nucleate erythroid cell, or a nucleated platelet precursor cell.

The one or more exogenous polypeptides (e.g. exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) may be genetically introduced into erythroid cell precursors, platelet precursor, or nucleated erythroid cells prior to terminal differentiation using a variety of DNA techniques, including transient or stable transfections and gene therapy approaches. The exogenous polypeptides may be expressed on the surface and/or in the cytoplasm of mature red blood cell or platelet.

Viral gene transfer may be used to transfect the cells with DNA encoding one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides). A number of viruses may be used as gene transfer vehicles including Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example (See, e.g., Osten et al., HEP 178:177-202 (2007)). Retroviruses, for example, efficiently transduce mammalian cells including human cells and integrate into chromosomes, conferring stable gene transfer.

One or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) may be transfected into an erythroid cell precursor, a platelet precursor, or a nucleated erythroid cell, expressed and subsequently retained and exhibited in a mature red blood cell or platelet. A suitable vector is the Moloney murine leukemia virus (MMLV) vector backbone (Malik et al., Blood 91:2664-2671 (1998)). Vectors based on MMLV, an oncogenic retrovirus, are currently used in gene therapy clinical trials (Hossle et al., News Physiol. Sci. 17:87-92 (2002)). For example, a DNA construct containing the cDNA encoding an exogenous polypeptide can be generated in the MMLV vector backbone using standard molecular biology techniques. The construct is transfected into a packaging cell line such as, for example, PA317 cells and the viral supernatant is used to transfect producer cells such as, for example, PG13 cells. The PG13 viral supernatant is incubated with an erythroid cell precursor, a platelet precursor, or a nucleated erythroid cell that has been isolated and cultured or has been freshly isolated as described herein. The expression of the exogenous polypeptide may be monitored using FACS analysis (fluorescence-activated cell sorting), for example, with a fluorescently labeled antibody directed against the exogenous polypeptide, if it is located on the surface of the aAPC. Similar methods may be used to express an exogenous polypeptide that is located in the inside of the aAPC.

Optionally, a fluorescent tracking molecule such as, for example, green fluorescent protein (GFP) may be transfected using a viral-based approach (Tao et al., Stem Cells 25:670-678 (2007)). Ecotopic retroviral vectors containing DNA encoding the enhanced green fluorescent protein (EGFP) or a red fluorescent protein (e.g., DsRed-Express) are packaged using a packaging cell such as, for example, the Phoenix-Eco cell line (distributed by Orbigen, San Diego, Calif.). Packaging cell lines stably express viral proteins needed for proper viral packaging including, for example, gag, pol, and env. Supernatants from the Phoenix-Eco cells into which viral particles have been shed are used to transduce e.g., erythroid cell precursors, platelet precursors, or a nucleated erythroid cells. In some instances, transduction may be performed on a specially coated surface such as, for example, fragments of recombinant fibronectin to improve the efficiency of retroviral mediated gene transfer (e.g., RetroNectin, Takara Bio USA, Madison, Wis.). Cells are incubated in RetroNectin-coated plates with retroviral Phoenix-Eco supernatants plus suitable co-factors. Transduction may be repeated the next day. In this instance, the percentage of cells expressing EGFP or DsRed-Express may be assessed by FACS. Other reporter genes that may be used to assess transduction efficiency include, for example, beta-galactosidase, chloramphenicol acetyltransferase, and luciferase as well as low-affinity nerve growth factor receptor (LNGFR), and the human cell surface CD24 antigen (Bierhuizen et al., Leukemia 13:605-613 (1999)).

Nonviral vectors may be used to introduce genetic material into suitable erythroid cells, platelets or precursors thereof to generate aAPCs. Nonviral-mediated gene transfer differs from viral-mediated gene transfer in that the plasmid vectors contain no proteins, are less toxic and easier to scale up, and have no host cell preferences. The “naked DNA” of plasmid vectors is by itself inefficient in delivering genetic material encoding a polypeptide to a cell and therefore is combined with a gene delivery method that enables entry into cells. A number of delivery methods may be used to transfer nonviral vectors into suitable erythroid cells, platelets or precursors thereof including chemical and physical methods.

A nonviral vector encoding one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) may be introduced into suitable erythroid cells, platelets or precursors thereof using synthetic macromolecules such as cationic lipids and polymers (Papapetrou et al., Gene Therapy 12:S118-S130 (2005)). Cationic liposomes, for example form complexes with DNA through charge interactions. The positively charged DNA/lipid complexes bind to the negative cell surface and are taken up by the cell by endocytosis. This approach may be used, for example, to transfect hematopoietic cells (See, e.g., Keller et al., Gene Therapy 6:931-938 (1999)). For erythroid cells, platelets or precursors thereof the plasmid DNA (approximately 0.5 μg in 25-100 μL of a serum free medium, such as, for example, OptiMEM (Invitrogen, Carlsbad, Calif.)) is mixed with a cationic liposome (approximately 4μg in 25 μL of serum free medium) such as the commercially available transfection reagent Lipofectamine™ (Invitrogen, Carlsbad, Calif.) and allowed to incubate for at least 20 min to form complexes. The DNA/liposome complex is added to suitable erythroid cells, platelets or precursors thereof and allowed to incubate for 5-24 h, after which time transgene expression of the polypeptide may be assayed. Alternatively, other commercially available liposome transfection agents may be used (e.g., In vivo GeneSHUTTLE., Qbiogene, Carlsbad, Calif.).

Optionally, a cationic polymer such as, for example, polyethylenimine (PEI) may be used to efficiently transfect erythroid cell progenitor cells, for example hematopoietic and umbilical cord blood-derived CD34+ cells (See, e.g., Shin et al., Biochim. Biophys. Acta 1725:377-384 (2005)). Human CD34+ cells are isolated from human umbilical cord blood and cultured in Iscove's modified Dulbecco's medium supplemented with 200 ng/ml stem cell factor and 20% heat-inactivated fetal bovine serum. Plasmid DNA encoding the exogenous polypeptide is incubated with branched or linear PEIs varying in size from 0.8 K to 750 K (Sigma Aldrich, Saint Louis, Mo., USA; Fermetas, Hanover, Md., USA). PEI is prepared as a stock solution at 4.2 mg/ml distilled water and slightly acidified to pH 5.0 using HCl. The DNA may be combined with the PEI for 30 min at room temperature at various nitrogen/phosphate ratios based on the calculation that 1 μg of DNA contains 3 nmol phosphate and 1 μl of PEI stock solution contains 10 nmol amine nitrogen. The isolated CD34+ cells are seeded with the DNA/cationic complex, centrifuged at 280×g for 5 min and incubated in culture medium for 4 or more h until gene expression of the polypeptide is assessed.

A plasmid vector may be introduced into suitable erythroid cells, platelets or precursors thereof using a physical method such as particle-mediated transfection, “gene gun”, biolistics, or particle bombardment technology (Papapetrou, et al., (2005) Gene Therapy 12:S118-S130). In this instance, DNA encoding the polypeptide is absorbed onto gold particles and administered to cells by a particle gun. This approach may be used, for example, to transfect erythroid progenitor cells, e.g., hematopoietic stem cells derived from umbilical cord blood (See, e.g., Verma et al., Gene Therapy 5:692-699 (1998)). As such, umbilical cord blood is isolated and diluted three fold in phosphate buffered saline. CD34+ cells are purified using an anti-CD34 monoclonal antibody in combination with magnetic microbeads coated with a secondary antibody and a magnetic isolation system (e.g., Miltenyi MiniMac System, Auburn, Calif., USA). The CD34+ enriched cells may be cultured as described herein. For transfection, plasmid DNA encoding the polypeptide is precipitated onto a particle, for example gold beads, by treatment with calcium chloride and spermidine. Following washing of the DNA-coated beads with ethanol, the beads may be delivered into the cultured cells using, for example, a Biolistic PDS-1000/He System (Bio-Rad, Hercules, Calif., USA). A reporter gene such as, for example, beta-galactosidase, chloramphenicol acetyltransferase, luciferase, or green fluorescent protein may be used to assess efficiency of transfection.

Optionally, electroporation methods may be used to introduce a plasmid vector into suitable erythroid cells, platelets or precursors thereof. Electroporation creates transient pores in the cell membrane, allowing for the introduction of various molecules into the cells including, for example, DNA and RNA as well as antibodies and drugs. As such, CD34+ cells are isolated and cultured as described herein. Immediately prior to electroporation, the cells are isolated by centrifugation for 10 min at 250×g at room temperature and resuspended at 0.2-10×10⁶ viable cells/ml in an electroporation buffer such as, for example, X-VIVO 10 supplemented with 1.0% human serum albumin (HSA). The plasmid DNA (1-50 μg) is added to an appropriate electroporation cuvette along with 500 μl of cell suspension. Electroporation may be done using, for example, an ECM 600 electroporator (Genetronics, San Diego, Calif., USA) with voltages ranging from 200 V to 280 V and pulse lengths ranging from 25 to 70 milliseconds. A number of alternative electroporation instruments are commercially available and may be used for this purpose (e.g., Gene Pulser XCELL, BioRad, Hercules, Calif.; Cellject Duo, Thermo Science, Milford, Mass.). Alternatively, efficient electroporation of isolated CD34+ cells may be performed using the following parameters: 4 mm cuvette, 1600 μF, 550 V/cm, and 10 μg of DNA per 500 μl of cells at 1×10⁵ cells/ml (Oldak et al., Acta Biochimica Polonica 49:625-632 (2002)).

Nucleofection, a form of electroporation, may also be used to transfect suitable erythroid cells, platelets or precursors thereof. In this instance, transfection is performed using electrical parameters in cell-type specific solutions that enable DNA (or other reagents) to be directly transported to the nucleus thus reducing the risk of possible degradation in the cytoplasm. For example, a Human CD34 CELL NYCLEOFECTOR Kit (from Amaxa Inc.) may be used to transfect suitable erythroid cells, platelets or precursors thereof. In this instance, 1-5×10⁶ cells in Human CD34 Cell NUCLEOFECTOR Solution are mixed with 1-5 μg of DNA and transfected in the NUCLEOFECTOR instrument using preprogrammed settings as determined by the manufacturer.

Erythroid cells, platelets or precursors thereof may be non-virally transfected with a conventional expression vector which is unable to self-replicate in mammalian cells unless it is integrated in the genome. Alternatively, erythroid cells, platelets or precursors thereof may be transfected with an episomal vector which may persist in the host nucleus as autonomously replicating genetic units without integration into chromosomes (Papapetrou et al., Gene Therapy 12:S118-S130 (2005)). These vectors exploit genetic elements derived from viruses that are normally extrachromosomally replicating in cells upon latent infection such as, for example, EBV, human polyomavirus BK, bovine papilloma virus-1 (BPV-1), herpes simplex virus-1 (HSV) and Simian virus 40 (SV40). Mammalian artificial chromosomes may also be used for nonviral gene transfer (Vanderbyl et al., Exp. Hematol. 33:1470-1476 (2005)).

Exogenous nucleic acids encoding one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) may be assembled into expression vectors by standard molecular biology methods known in the art, e.g., restriction digestion, overlap-extension PCR, and Gibson assembly.

Exogenous nucleic acids may comprise a gene encoding one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) that are not normally expressed on the cell surface, e.g., of an erythroid cell, fused to a gene that encodes an endogenous or native membrane protein, such that the exogenous polypeptide is expressed on the cell surface. For example, a exogenous gene encoding an exogenous antigenic polypeptide can be cloned at the N terminus following the leader sequence of a type 1 membrane protein, at the C terminus of a type 2 membrane protein, or upstream of the GPI attachment site of a GPI-linked membrane protein.

Standard cloning methods can be used to introduce flexible amino acid linkers between two fused genes. For example, the flexible linker is a poly-glycine poly-serine linker such as [Gly4Ser]3_(SEQ ID NO: 1) commonly used in generating single-chain antibody fragments from full-length antibodies (Antibody Engineering: Methods & Protocols, Lo 2004), or ala-gly-ser-thr polypeptides such as those used to generate single-chain Arc repressors (Robinson & Sauer, PNAS 1998). In some embodiments, the flexible linker provides the polypeptide with more flexibility and steric freedom than the equivalent construct without the flexible linker.

An epitope tag may be placed between two fused genes, such as, e.g., a nucleic acid sequence encoding an HA epitope tag—amino acids YPYDVPDYA (SEQ ID NO: 2), a CMyc tag—amino acids EQKLISEEDL (SEQ ID NO: 3), or a Flag tag—amino acids DYKDDDDK (SEQ ID NO: 4). The epitope tag may be used for the facile detection and quantification of expression using antibodies against the epitope tag by flow cytometry, western blot, or immunoprecipitation.

In some embodiments, the aAPC comprises one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) and at least one other heterologous polypeptide. The at least one other heterologous polypeptide can be a fluorescent protein. The fluorescent protein can be used as a reporter to assess transduction efficiency. In some embodiments, the fluorescent protein is used as a reporter to assess expression levels of the exogenous polypeptide if both are made from the same transcript. In some embodiments, the at least one other polypeptide is heterologous and provides a function, such as, e.g., multiple antigens, multiple capture targets, enzyme cascade. In some embodiments, the recombinant nucleic acid comprises a gene encoding an antigenic polypeptide and a second gene, wherein the second gene is separated from the gene encoding the antigenic polypeptide by a viral-derived T2A sequence (gagggcagaggaagtcttctaacatgcggtgacgtggaggsgsstcccggccct (SEQ ID NO: 5)) that is post-translationally cleaved into two mature proteins.

In some embodiments, the exogenous nucleic acid encoding an exogenous antigen-presenting polypeptide comprises a gene sequence for a MHC cell surface protein that is fused to the 3′ end of the sequence for Kell and amplified using PCR. In some embodiments, the exogenous nucleic acid encoding an exogenous antigen-presenting polypeptide comprises a gene sequence for a MHC cell surface protein that is fused to a poly-glycine/serine linker, followed by the 3′ end of the sequence for Kell, and amplified using PCR. In some embodiments, the exogenous nucleic acid encoding an exogenous antigen-presenting polypeptide comprises the 3′ end of a gene sequence for a MHC cell surface protein that is fused to an epitope tag sequence, of which may be one, or a combination of, an; HA-tag, Green fluorescent protein tag, Myc-tag, chitin binding protein, maltose binding protein, glutathione-S-transferase, poly(His)tag, thioredoxin, poly(NANP), FLAG-tag, V5-tag, AviTag, Calmodulin-tag, polyglutamate-tag, E-tag, S-tag, SBP-tag, Softag-1, Softag-3, Strep-tag, TC-tag, VSV-tag, Xpress-tag, Isopeptag, SpyTag, biotin carboxyl carrier protein, Nus-tag, Fc-tag, or Ty-tag. The entire construct is fused to the 3′ end of the sequence for Kell and then amplified using PCR. The exogenous gene constructs encoding the various exogenous antigen presenting polypeptides are, for example, subsequently loaded into a lentiviral vector and used to transduce a cell population.

In some embodiments, a population of erythroid cells is incubated with lentiviral vectors comprising exogenous nucleic acid encoding one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides), specific plasmids of which may include; pLKO.1 puro, PLKO.1-TRC cloning vector, pSico, FUGW, pLVTHM, pLJM1, pLion11, pMD2.G, pCMV-VSV-G, pCI-VSVG, pCMV-dR8.2 dvpr, psPAX2, pRSV-Rev, and pMDLg/pRRE to generate an aAPC. The vectors may be administered at 10, 100, 1,000, 10,000 pfu and incubated for 12 hrs.

In certain embodiments, the aAPC is an erythroid cell that presents, e.g. comprises on the cell surface, an exogenous antigen-presenting polypeptide that is conjugated to one or more exogenous antigenic polypeptides. In other embodiments, the aAPC is an erythroid cell that presents, e.g. comprises on the cell surface, an exogenous antigen-presenting polypeptide, an exogenous antigenic polypeptide and at least one exogenous costimulatory polypeptide that is part of a conjugate pair. In other embodiments, the aAPC is an erythroid cell that presents, e.g. comprises on the cell surface, an exogenous antigen-presenting polypeptide, an exogenous antigenic polypeptide and at least one exogenous co-inhibitory polypeptide that is part of a conjugate pair. In some embodiments, the erythroid cell is an enucleated cell. In some embodiments, the erythroid cell is a nucleated cell.

Conjugation may be achieved chemically or enzymatically. Chemical conjugation may be accomplished by covalent bonding of the exogenous antigen-presenting polypeptide to one or more exogenous antigenic polypeptides, with or without the use of a linker. Chemical conjugation may be accomplished by the covalent bonding of a costimulatory polypeptide and a binding pair member, with or without the use of a linker. Chemical conjugation may be accomplished by the covalent bonding of a coinhibitory polypeptide and a binding pair member, with or without the use of a linker. The formation of such conjugates is within the skill of artisans and various techniques are known for accomplishing the conjugation, with the choice of the particular technique being guided by the materials to be conjugated. The addition of amino acids to the polypeptide (C- or N-terminal) which contain ionizable side chains, e.g., aspartic acid, glutamic acid, lysine, arginine, cysteine, histidine, or tyrosine, and are not contained in the active portion of the polypeptide sequence, serve in their unprotonated state as a potent nucleophile to engage in various bioconjugation reactions with reactive groups attached to polymers, e.g., homo- or hetero-bi-functional PEG (e.g., Lutolf and Hubbell, Biomacromolecules 2003; 4:713-22, Hermanson, Bioconjugate Techniques, London. Academic Press Ltd; 1996).

In an embodiment, the exogenous antigen-presenting polypeptide may be bound to one or more exogenous antigenic polypeptides through a biotin-streptavidin bridge. In other embodiments, the costimulatory polypeptide and a binding pair member are bound through a biotin-streptavidin bridge. In other embodiments, the coinhibitory polypeptide and a binding pair member are bound through a biotin-streptavidin bridge.

For example, a biotinylated antigenic polypeptide may be linked to a non-specifically biotinylated surface of the exogenous antigen-presenting polypeptide through a streptavidin bridge. Biotin conjugation can occur by a number of chemical means (See, e.g., Hirsch et al., Methods Mol. Biol. 295: 135-154 (2004)). The exogenous antigen-presenting polypeptide may be biotinylated using an amine reactive biotinylation reagent such as, for example, EZ-Link Sulfo-NHS-SS-Biotin (sulfosuccinimidyl 2-(biotinamido)-ethyl-1,3-dithiopropionate; Pierce-Thermo Scientific, Rockford, Ill., USA; See, e.g., Jaiswal et al., Nature Biotech. 21:47-51 (2003)). For example, isolated erythroid cells may be incubated for 30 min at 4° C. in 1 mg/ml solution of sulfo-NHS-SS in phosphate-buffered saline. Excess biotin reagent is removed by washing the cells with Tris-buffered saline. The biotinylated cells are then reacted with the biotinylated exogenous antigenic polypeptide in the presence of streptavidin to form an aAPC presenting an exogenous antigen-presenting polypeptide that is bound to one or more exogenous antigenic polypeptides through a biotin-streptavidin bridge.

Erythroid cells described herein can also be produced using coupling reagents to link an exogenous polypeptide to a cell. For instance, click chemistry can be used. Coupling reagents can be used to couple an exogenous polypeptide to a cell, for example, when the exogenous polypeptide is a complex or difficult to express polypeptide, e.g., a polypeptide, e.g., a multimeric polypeptide; large polypeptide; polypeptide derivatized in vitro; an exogenous polypeptide that may have toxicity to, or which is not expressed efficiently in, the erythroid cells. Click chemistry and other conjugation methods for functionalizing erythroid cells is described in International Application No. PCT/US2018/000042, which claims priority to U.S. Provisional Application No. 62/460,589, filed Feb. 17, 2017 and U.S. Provisional Application No. 62/542,142, filed Jul. 8, 2017.

Thus, in some embodiments, an erythroid cell described herein comprises many as, at least, more than, or about 5,000, 10,000, 50,000, 100,000, 200,000, 300,000, 400,000, 500,000 coupling reagents per cell. In some embodiments, the erythroid cells are made by a method comprising a) coupling a first coupling reagent to an erythroid cell, thereby making a pharmaceutical preparation, product, or intermediate. In an embodiment, the method further comprises: b) contacting the cell with an exogenous polypeptide coupled to a second coupling reagent e.g., under conditions suitable for reaction of the first coupling reagent with the second coupling reagent. In embodiments, two or more exogenous polypeptides are coupled to the cell (e.g., using click chemistry). In embodiments, a first exogenous polypeptide is coupled to the cell (e.g., using click chemistry) and a second exogenous polypeptide comprises a polypeptide expressed from an exogenous nucleic acid.

In some embodiments, one or more coupling reagents are used to couple the one or more exogenous polypeptides to the erythroid cell described herein. In some embodiments, two or more coupling reagents are used to couple the two or more exogenous polypeptides to the erythroid cell described herein. In some embodiments, two or more exogenous polypeptides are coupled to the cell (e.g., using click chemistry) using the same coupling reagent. In some embodiments, two or more exogenous polypeptides are coupled to the cell (e.g., using click chemistry) using different coupling reagents. In some embodiments, a first exogenous polypeptide is coupled to the cell (e.g., using click chemistry) using a first coupling reagent, and a second exogenous polypeptide is coupled to the cell (e.g., using click chemistry) using a second coupling reagent. In some embodiments, three or more exogenous polypeptides are coupled to the cell (e.g., using click chemistry) using the same coupling reagent. In some embodiments, three or more exogenous polypeptides are coupled to the cell (e.g., using click chemistry) using different coupling reagents. In some embodiments, a first exogenous polypeptide is coupled to the cell (e.g., using click chemistry) using a first coupling reagent, a second exogenous polypeptide is coupled to the cell (e.g., using click chemistry) using the first coupling reagent, and a third exogenous polypeptide is coupled to the cell (e.g., using click chemistry) using a second coupling reagent. In some embodiments, a first exogenous polypeptide is coupled to the cell (e.g., using click chemistry) using a first coupling reagent, a second exogenous polypeptide is coupled to the cell (e.g., using click chemistry) using a second coupling reagent, and a third exogenous polypeptide is coupled to the cell (e.g., using click chemistry) using the first coupling reagent. In some embodiments, a first exogenous polypeptide is coupled to the cell (e.g., using click chemistry) using a first coupling reagent, a second exogenous polypeptide is coupled to the cell (e.g., using click chemistry) using the a second coupling reagent, and a third exogenous polypeptide is coupled to the cell (e.g., using click chemistry) using the second coupling reagent.

In an embodiment, one or more coupling reagents are used to couple the one or more exogenous polypeptides to the erythroid cell described herein, wherein the erythroid cell is a human erythroid cell. In some embodiments, the erythroid cell is a human cell, and two or more coupling agents are used to couple the two or more exogenous polypeptides to the erythroid cell. In some embodiments, the erythroid cell is a human cell, and two or more exogenous polypeptides are coupled to the cell (e.g., using click chemistry) using the same coupling reagent. In some embodiments, the erythroid cell is a human cell, and two or more exogenous polypeptides are coupled to the cell (e.g., using click chemistry) using different coupling reagents. In some embodiments, the erythroid cell is a human cell, and a first exogenous polypeptide is coupled to the cell (e.g., using click chemistry) using a first coupling reagent, and a second exogenous polypeptide is coupled to the cell (e.g., using click chemistry) using a second coupling reagent. In some embodiments, the erythroid cell is a human cell, and three or more exogenous polypeptides are coupled to the cell (e.g., using click chemistry) using the same coupling reagent. In some embodiments, the erythroid cell is a human cell, and three or more exogenous polypeptides are coupled to the cell (e.g., using click chemistry) using the different coupling reagents. In some embodiments, the erythroid cell is a human cell, and a first exogenous polypeptide is coupled to the cell (e.g., using click chemistry) using a first coupling reagent, a second exogenous polypeptide is coupled to the cell (e.g., using click chemistry) using the first coupling reagent, and a third exogenous polypeptide is coupled to the cell (e.g., using click chemistry) using a second coupling reagent. In some embodiments, the erythroid cell is a human cell, and a first exogenous polypeptide is coupled to the cell (e.g., using click chemistry) using a first coupling reagent, a second exogenous polypeptide is coupled to the cell (e.g., using click chemistry) using a second coupling reagent, and a third exogenous polypeptide is coupled to the cell (e.g., using click chemistry) using the first coupling reagent. In some embodiments, the erythroid cell is a human cell, and a first exogenous polypeptide is coupled to the cell (e.g., using click chemistry) using a first coupling reagent, a second exogenous polypeptide is coupled to the cell (e.g., using click chemistry) using a second coupling reagent, and a third exogenous polypeptide is coupled to the cell (e.g., using click chemistry) using the second coupling reagent.

In embodiments, it is an advantage of the present disclosure to employ two different coupling reagents to click three or more exogenous polypeptides to an erythroid cell, and in particular to a human erythroid cell. Without being bound by theory, the present disclosure contemplates that in erythroid cells comprising three or more exogenous polypeptides coupled to the cell (e.g. using click chemistry) using different coupling reagents, that competition between the exogenous polypeptides for the coupling reagents is reduced, and as a result the copy numbers of the exogenous polypeptides are maximized.

In some embodiments, the coupling reagent comprises an azide coupling reagent. In some embodiments, the azide coupling reagent comprises an azidoalkyl moiety, azidoaryl moiety, or an azidoheteroaryl moiety. Exemplary azide coupling reagents include 3-azidopropionic acid sulfo-NHS ester, azidoacetic acid NHS ester, azido-PEG-NHS ester, azidopropylamine, azido-PEG-amine, azido-PEG-maleimide, bis-sulfone-PEG-azide, or a derivative thereof. Coupling reagents may also comprise an alkene moiety, e.g., a transcycloalkene moiety, an oxanorbornadiene moiety, or a tetrazine moiety. Additional coupling reagents can be found in Click Chemistry Tools (https://clickchemistrytools.com/) or Lahann, J (ed) (2009) Click Chemistry for Biotechnology and Materials Science, each of which is incorporated herein by reference in its entirety.

In another embodiment, the antigenic polypeptide is attached to the cell, e.g., an erythroid cell, via a covalent attachment to generate an aAPC comprising an erythroid cell presenting one or more exogenous antigenic polypeptides (e.g. a first exogenous antigenic polypeptide, or a first antigenic polypeptide and a second exogenous antigenic polypeptide). For example, the antigenic polypeptide may be derivatized and bound to the erythroid cell or platelet using a coupling compound containing an electrophilic group that will react with nucleophiles on the erythroid cell or platelet to form the interbonded relationship. Representative of these electrophilic groups are αβ unsaturated carbonyls, alkyl halides and thiol reagents such as substituted maleimides. In addition, the coupling compound can be coupled to an antigenic polypeptide via one or more of the functional groups in the polypeptide such as amino, carboxyl and tryosine groups. For this purpose, coupling compounds should contain free carboxyl groups, free amino groups, aromatic amino groups, and other groups capable of reaction with enzyme functional groups. Highly charged antigenic polypeptides can also be prepared for immobilization on, e.g., erythroid cells or platelets through electrostatic bonding to generate aAPCs. Examples of these derivatives would include polylysyl and polyglutamyl enzymes.

The choice of the reactive group embodied in the derivative depends on the reactive conditions employed to couple the electrophile with the nucleophilic groups on the erythroid cell or platelet for immobilization. A controlling factor is the desire not to inactivate the coupling agent prior to coupling of the exogenous polypeptide immobilized by the attachment to the erythroid cell or platelet. Such coupling immobilization reactions can proceed in a number of ways. Typically, a coupling agent can be used to form a bridge between the exogenous polypeptide and the erythroid cell or platelet. In this case, the coupling agent should possess a functional group such as a carboxyl group which can be caused to react with the exogenous polypeptide. One way of preparing the exogenous polypeptide for conjugation includes the utilization of carboxyl groups in the coupling agent to form mixed anhydrides which react with the exogenous polypeptide, in which use is made of an activator which is capable of forming the mixed anhydride. Representative of such activators are isobutylchloroformate or other chloroformates which give a mixed anhydride with coupling agents such as 5,5′-(dithiobis(2-nitrobenzoic acid) (DTNB), p-chloromercuribenzoate (CMB), or m-maleimidobenzoic acid (MBA). The mixed anhydride of the coupling agent reacts with the exogenous polypeptide to yield the reactive derivative which in turn can react with nucleophilic groups on the erythroid cell or platelet to immobilize the exogenous polypeptide.

Functional groups on an antigenic polypeptide, such as carboxyl groups can be activated with carbodiimides and the like activators. Subsequently, functional groups on the bridging reagent, such as amino groups, will react with the activated group on the exogenous polypeptide to form the reactive derivative. In addition, the coupling agent should possess a second reactive group which will react with appropriate nucleophilic groups on the erythroid cell or platelet to form the bridge. Typical of such reactive groups are alkylating agents such as iodoacetic acid, αβ unsaturated carbonyl compounds, such as acrylic acid and the like, thiol reagents, such as mercurials, substituted maleimides and the like.

Alternatively, functional groups on the antigenic polypeptide can be activated so as to react directly with nucleophiles on, e.g., erythroid cells or platelets to obviate the need for a bridge-forming compound. For this purpose, use is made of an activator such as Woodward's Reagent K or the like reagent which brings about the formation of carboxyl groups in the exogenous polypeptide into enol esters, as distinguished from mixed anhydrides. The enol ester derivatives of exogenous polypeptides subsequently react with nucleophilic groups on, e.g., an erythroid cell or platelet to effect immobilization of the antigenic polypeptide, thereby creating an aAPC.

In some embodiments, the aAPC comprising an erythroid cell presenting (e.g. comprising on the cell surface) one or more exogenous antigenic polypeptides is generated by contacting an erythroid cell with an antigenic polypeptide and optionally a payload, wherein contacting does not include conjugating the antigenic polypeptide to the erythroid cell using an attachment site comprising Band 3 (CD233), aquaporin-1, Glut-1, Kidd antigen, RhAg/Rli50 (CD241), Rli (CD240), Rh30CE (CD240CE), Rh30D (CD240D), Kx, glycophorin B (CD235b), glycophorin C (CD235c), glycophorin D (CD235d), Kell (CD238), Duffy/DARCi (CD234), CR1 (CD35), DAF (CD55), Globoside, CD44, ICAM-4 (CD242), Lu/B-CAM (CD239), XG1/XG2 (CD99), EMMPRIN/neurothelin (CD147), JMH, Glycosyltransferase, Cartwright, Dombrock, C4A/CAB, Scianna, MER2, stomatin, BA-1 (CD24), GPIV (CD36), CD108, CD139, or H antigen (CD173). In some embodiments, the erythroid cell is an enucleated cell. In some embodiments, the erythroid cell is a nucleated cell.

In some embodiments, the aAPC comprises an erythroid cell presenting (e.g. comprising on the cell surface) one or more exogenous antigenic polypeptides, wherein the one or more exogenous antigenic polypeptides are enzymatically conjugated.

In specific embodiments, the antigenic polypeptide can be conjugated to the surface of, e.g., an erythroid cell or platelet by various chemical and enzymatic means, including but not limited to chemical conjugation with bifunctional cross-linking agents such as, e.g., an NHS ester-maleimide heterobifunctional crosslinker to connect a primary amine group with a reduced thiol group. These methods also include enzymatic strategies such as, e.g., transpeptidase reaction mediated by a sortase enzyme to connect one polypeptide containing the acceptor sequence LPXTG (SEQ ID NO: 6) or LPXTA (SEQ ID NO: 7) with a polypeptide containing the N-terminal donor sequence GGG, see e.g., Swee et al., PNAS 2013. The methods also include combination methods, such as e.g., sortase-mediated conjugation of Click Chemistry handles (an azide and an alkyne) on the antigen and the cell, respectively, followed by a cyclo-addition reaction to chemically bond the antigen to the cell, see e.g., Neves et al., Bioconjugate Chemistry, 2013. Sortase-mediated modification of proteins is described in International Application No. PCT/US2014/037545 and International Application No. PCT/US2014/037554, both of which are incorporated by reference in their entireties herein.

In some embodiments, a protein is modified by the conjugation of a sortase substrate comprising an amino acid, a peptide, a protein, a polynucleotide, a carbohydrate, a tag, a metal atom, a contrast agent, a catalyst, a non-polypeptide polymer, a recognition element, a small molecule, a lipid, a linker, a label, an epitope, an antigen, a therapeutic agent, a toxin, a radioisotope, a particle, or moiety comprising a reactive chemical group, e.g., a click chemistry handle.

If desired, a catalytic bond-forming polypeptide domain can be expressed on or in e.g., an erythroid cell or platelet, either intracellularly or extracellularly. Many catalytic bond-forming polypeptides exist, including transpeptidases, sortases, and isopeptidases, including those derived from Spy0128, a protein isolated from Streptococcus pyogenes.

In some embodiments, any of the polypeptides described herein are not conjugated to the cell using a sortase

It has been demonstrated that splitting the autocatalytic isopeptide bond-forming subunit (CnaB2 domain) of Spy0128 results in two distinct polypeptides that retain catalytic activity with specificity for each other. The polypeptides in this system are termed SpyTag and SpyCatcher. Upon mixing, SpyTag and SpyCatcher undergo isopeptide bond formation between Asp117 on SpyTag and Lys31 on SpyCatcher (Zakeri and Howarth, JACS 2010, 132:4526). The reaction is compatible with the cellular environment and highly specific for protein/peptide conjugation (Zakeri, B.; Fierer, J. O.; Celik, E.; Chittock, E. C.; Schwarz-Linek, U.; Moy, V. T.; Howarth, M. Proc. Natl. Acad. Sci. U.S.A. 2012, 109, E690-E697). SpyTag and SpyCatcher has been shown to direct post-translational topological modification in elastin-like protein. For example, placement of SpyTag at the N-terminus and SpyCatcher at the C-terminus directs formation of circular elastin-like proteins (Zhang et al, Journal of the American Chemical Society, 2013).

The components SpyTag and SpyCatcher can be interchanged such that a system in which molecule A is fused to SpyTag and molecule B is fused to SpyCatcher is functionally equivalent to a system in which molecule A is fused to SpyCatcher and molecule B is fused to SpyTag. For the purposes of this document, when SpyTag and SpyCatcher are used, it is to be understood that the complementary molecule could be substituted in its place.

A catalytic bond-forming polypeptide, such as a SpyTag/SpyCatcher system, can be used to attach the antigenic polypeptide to the surface of, e.g., an erythroid cell, such as an engineered erythroid cell, to generate an aAPC. The SpyTag polypeptide sequence can be expressed on the extracellular surface of the erythroid cell. The SpyTag polypeptide can be, for example, fused to the N terminus of a type-1 or type-3 transmembrane protein, e.g., glycophorin A, fused to the C terminus of a type-2 transmembrane protein, e.g., Kell, inserted in-frame at the extracellular terminus or in an extracellular loop of a multi-pass transmembrane protein, e.g., Band 3, fused to a GPI-acceptor polypeptide, e.g., CD55 or CD59, fused to a lipid-chain-anchored polypeptide, or fused to a peripheral membrane protein. The nucleic acid sequence encoding the SpyTag fusion can be expressed within an aAPC. An antigenic polypeptide can be fused to SpyCatcher. The nucleic acid sequence encoding the SpyCatcher fusion can be expressed and secreted from the same erythroid cell that expresses the SpyTag fusion. Alternatively, the nucleic acid sequence encoding the SpyCatcher fusion can be produced exogenously, for example in a bacterial, fungal, insect, mammalian, or cell-free production system. Upon reaction of the SpyTag and SpyCatcher polypeptides, a covalent bond will be formed that attaches the antigenic polypeptide to the surface of the erythroid cell to form an aAPC. An erythroid cell comprising the antigenic polypeptide fusion is an example of an aAPC that comprises a conjugated antigenic polypeptide.

In some embodiments, the SpyTag polypeptide may be expressed as a fusion to the N terminus of glycophorin A under the control of the Gatal promoter in an erythroid cell. An exogenous polypeptide (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides), fused to the SpyCatcher polypeptide sequence can be expressed under the control of the Gatal promoter in the same erythroid cell. Upon expression of both fusion polypeptides, an isopeptide bond will be formed between the SpyTag and SpyCatcher polypeptides, forming a covalent bond between the erythroid cell surface and the exogenous polypeptide.

In another embodiment, the SpyTag polypeptide may be expressed as a fusion to the N terminus of glycophorin A under the control of the Gatal promoter in an erythroid cell. An exogenous polypeptide (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) fused to the SpyCatcher polypeptide sequence can be expressed in a suitable mammalian cell expression system, for example HEK293 cells. Upon expression of the SpyTag fusion polypeptide on the erythroid cell, the SpyCatcher fusion polypeptide can be brought in contact with the cell. Under suitable reaction conditions, an isopeptide bond will be formed between the SpyTag and SpyCatcher polypeptides, forming a covalent bond between the erythroid cell surface and the antigenic polypeptide. An erythroid cell comprising the antigenic polypeptide fusion is an example of an aAPC that comprises a conjugated antigenic polypeptide.

Other molecular fusions may be formed between exogenous polypeptides and include direct or indirect conjugation. The exogenous polypeptides may be directly conjugated to each other or indirectly through a linker. The linker may be a peptide, a polymer, an aptamer, or a nucleic acid. The polymer may be, e.g., natural, synthetic, linear, or branched. Antigenic polypeptides can comprise a heterologous fusion protein that comprises a first polypeptide and a second polypeptide with the fusion protein comprising the polypeptides directly joined to each other or with intervening linker sequences and/or further sequences at one or both ends. The conjugation to the linker may be through covalent bonds or ionic bonds.

In certain embodiments, the polypeptide is loaded into the aAPC comprising an erythroid cell. In some embodiments, the polypeptide is loaded into the aAPC comprising an enucleated cell. In some embodiments, the polypeptide is loaded into the aAPC comprising a nucleated cell. In some embodiments, aAPCs comprising erythroid or enucleated cells are generated by loading, e.g., erythroid cells or platelets with one or more exogenous polypeptides, such that the one or more exogenous polypeptides are internalized within the erythroid cells or platelets. Optionally, the erythroid cells or platelets may additionally be loaded with a payload, such as, e.g., a therapeutic agent.

A number of methods may be used to load, e.g., erythroid cells or platelets with an exogenous polypeptide. Suitable methods include, for example, hypotonic lysis, hypotonic dialysis, osmosis, osmotic pulsing, osmotic shock, ionophoresis, electroporation, sonication, microinjection, calcium precipitation, membrane intercalation, lipid mediated transfection, detergent treatment, viral infection, diffusion, receptor mediated endocytosis, use of protein transduction domains, particle firing, membrane fusion, freeze-thawing, mechanical disruption, and filtration. Any one such method or a combination thereof may be used to generate the aAPCs comprising an engineered erythroid cell presenting (e.g. comprising on the cell surface) one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) described herein.

For hypotonic lysis, e.g., erythroid cell are exposed to low ionic strength buffer causing them to burst. The exogenous polypeptide distributes within the cells. Erythroid cell, specifically red blood cells may be hypotonically lysed by adding 30-50 fold volume excess of 5 mM phosphate buffer (pH 8) to a pellet of isolated red blood cells. The resulting lysed cell membranes are isolated by centrifugation. The pellet of lysed red blood cell membranes is resuspended and incubated in the presence of the exogenous polypeptide in a low ionic strength buffer, e.g., for 30 min. Alternatively, the lysed red blood cell membranes may be incubated with the exogenous polypeptide for as little as one minute or as long as several days, depending upon the best conditions determined to efficiently load the erythroid cells.

Alternatively, erythroid cells, specifically red blood cells (e.g. erythrocytes) may be loaded with an exogenous polypeptide using controlled dialysis against a hypotonic solution to swell the cells and create pores in the cell membrane (See, e.g., U.S. Pat. Nos. 4,327,710; 5,753,221; and 6,495,351). For example, a pellet of isolated red blood cells is resuspended in 10 mM HEPES, 140 mM NaCl, 5 mM glucose pH 7.4 and dialyzed against a low ionic strength buffer containing 10 mM NaH₂PO₄, 10 mM NaHCO₃, 20 mM glucose, and 4 mM MgCl₂, pH 7.4. After 30-60 min, the red blood cells are further dialyzed against 16 mM NaH₂PO₄, pH 7.4 solution containing the exogenous polypeptide for an additional 30-60 min. All of these procedures may be advantageously performed at a temperature of 4° C. In some instances, it may be beneficial to load a large quantity of erythroid cells, specifically red blood cells by a dialysis approach and a specific apparatus designed for this purpose may be used (See, e.g., U.S. Pat. Nos. 4,327,710, 6,139,836 and 6,495,351 B2).

The loaded erythroid cells, specifically red blood cells can be resealed by gentle heating in the presence of a physiological solution such as, for example, 0.9% saline, phosphate buffered saline, Ringer's solution, cell culture medium, blood plasma or lymphatic fluid. For example, well-sealed membranes may be generated by treating the disrupted erythroid cells, specifically red blood cells for 1-2 min in 150 mM salt solution of, for example, 100 mM phosphate (pH 8.0) and 150 mM sodium chloride at a temperature of 60° C. Alternatively, the cells may be incubated at a temperature of 25-50° C. for 30 min to 4 h (See, e.g., U.S. Patent Application 2007/0243137 A1). Alternatively, the disrupted red blood cells may be resealed by incubation in 5 mM adenine, 100 mM inosine, 2 mM ATP, 100 mM glucose, 100 mM Na-pyruvate, 4 mM MgCl2, 194 mM NaCl, 1.6 M KCl, and 35 mM NaH₂PO₄, pH 7.4 at a temperature of 37° C. for 20-30 min (See, e.g., U.S. Pat. No. 5,753,221).

For electroporation, e.g., erythroid cells or platelets are exposed to an electrical field which causes transient holes in the cell membrane, allowing the one or more exogenous polypeptides (e.g. exogenous antigenic polypeptides, exogenous antigen-presenting polypeptides, exogenous costimulatory polypeptides, exogenous T cell expansion polypeptides, exogenous co-inhibitory polypeptides, exogenous proliferation inhibitors, exogenous amino acid-depleting polypeptides, exogenous regulatory T cell expansion polypeptides, exogenous placeholder polypeptides) to diffuse into the cell (See, e.g., U.S. Pat. No. 4,935,223). Erythroid cells, specifically red blood cells, for example, are suspended in a physiological and electrically conductive media such as platelet-free plasma to which the one or more exogenous polypeptides are added. The mixture in a volume ranging from 0.2 to 1.0 ml is placed in an electroporation cuvette and cooled on ice for 10 min. The cuvette is placed in an electroporation apparatus such as, for example, an ECM 830 (from BTX Instrument Division, Harvard Apparatus, Holliston, Mass.). The cells are electroporated with a single pulse of approximately 2.4 milliseconds in length and a field strength of approximately 2.0 kV/cm. Alternatively, electroporation of erythroid cells, specifically red blood cells may be carried out using double pulses of 2.2 kV delivered at 0.25.mu.F using a Bio-Rad Gene Pulsar apparatus (Bio-Rad, Hercules, Calif., USA) to achieve a loading capacity of over 60% (Flynn et al., Cancer Lett. 82:225-229 (1994)). The cuvette is returned to the ice bath for 10-60 min and then placed in a 37° C. water bath to induce resealing of the cell membrane. Any suitable electroporation method may be used to generate the aAPCs described herein.

For sonication, erythroid cells are, for example, exposed to high intensity sound waves, causing transient disruption of the cell membrane allowing the one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) to diffuse into the cell. Any suitable sonication method may be used to generate the aAPCs described herein.

For detergent treatment, erythroid cells, for example, are treated with a mild detergent which transiently compromises the cell membrane by creating holes through which the one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) may diffuse. After cells are loaded, the detergent is washed from the cells. For example, the detergent may be saponin. Any suitable detergent treatment method may be used to generate the aAPCs described herein.

For receptor mediated endocytosis, erythroid cells, for example, may have a surface receptor which upon binding of the one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) induces internalization of the receptor and the associated exogenous polypeptides. Any suitable endocytosis method may be used to generate an aAPC comprising an erythroid cell presenting (e.g. comprising on the cell surface) one or more exogenous polypeptides, as described herein.

For mechanical firing, erythroid cells, for example, may be bombarded with the one or more exogenous polypeptides (e.g., exogenous antigenic polypeptides, exogenous antigen-presenting polypeptides, exogenous costimulatory polypeptides, exogenous coinhibitory polypeptides, exogenous amino acid-depleting polypeptides, and exogenous Treg costimulatory polypeptides) attached to a heavy or charged particle such as, for example, gold microcarriers and are mechanically or electrically accelerated such that they traverse the cell membrane. Microparticle bombardment may be achieved using, for example, the Helios Gene Gun (from, e.g., Bio-Rad, Hercules, Calif., USA). Any suitable microparticle bombardment method may be used to generate the aAPCs described herein.

For filtration, erythroid cells or platelets and the exogenous polypeptides may be forced through a filter of pore size smaller than the cell causing transient disruption of the cell membrane and allowing the exogenous polypeptides to enter the cell. Any suitable filtration method may be used to generate the aAPCs comprising engineered erythroid cells presenting (e.g. comprising on the cell surface) exogenous polypeptides as described herein.

For freeze thawing, erythroid cells are subjected to several freeze thaw cycles, resulting in cell membrane disruption (See, e.g., U.S. Patent Application 2007/0243137 A1). In this instance, a pellet of packed red blood cells (0.1-1.0 ml) is mixed with an equal volume (0.1-1.0 ml) of an isotonic solution (e.g., phosphate buffered saline) containing the one or more exogenous polypeptides. The red blood cells are frozen by immersing the tube containing the cells and one or more exogenous polypeptides into liquid nitrogen. Alternatively, the cells may be frozen by placing the tube in a freezer at −20° C. or −80° C. The cells are then thawed in, e.g., a 23° C. water bath and the cycle repeated if necessary to increase loading. Any suitable freeze-thaw method may be used to generate the aAPCs comprising engineered erythroid cells presenting (e.g. comprising on the cell surface) exogenous polypeptides as described herein.

Exogenous polypeptides can be detected on the aAPC. The presence of the exogenous polypeptide can be validated and quantified using standard molecular biology methods, e.g., Western blotting or FACS analysis. Exogenous polypeptides present in the intracellular environment may be quantified upon cell lysis or using fluorescent detection.

In some embodiments of the above aspects and embodiments, the erythroid cell is an enucleated cell. In some embodiments of the above aspects and embodiments, the erythroid cell is a nucleated cell.

Vehicles for Polypeptides Described Herein

In one aspect, one or more polypeptides described herein are loaded onto, attached (e.g., immobilized or conjugated) to the surface of, and/or enclosed in a non-cellular delivery vehicle. The non-cellular delivery vehicle can be, for example, a nanolipidgel, a polymeric particle, an agarose particle, a latex particle, a silica particle, a liposome, or a multilamellar vesicles. In some embodiments, the non-cellular delivery vehicle comprises or consists of a nanoparticle of from about 1 nm to about 900 nm in diameter. In some embodiments, the non-cellular delivery vehicle comprises an average diameter of from about 0.1 to about 20 microns (such as from about 0.5 microns to about 10 microns, e.g., about 5 microns or less (e.g., about 2.5 to about 5 microns)). In some embodiments, the non-cellular delivery vehicle comprises an average diameter of from about 1 μm to about 10 μm. In some embodiments, the non-cellular delivery vehicle comprises a biodegradable polymer. In some embodiments, the non-cellular delivery vehicle comprises a natural polymer. In some embodiments, the non-cellular delivery vehicle comprises a synthetic polymer. Representative polymers include, but are not limited to, a poly(hydroxy acid), a polyhydroxyalkanoate, a polycaprolactone, a polycarbonate, a polyamide, a polyesteramide, poly(acrylamide), poly(ester), poly(alkylcyanoacrylates), poly(lactic acid) (PLA), poly(glycolic acids) (PGA), and poly(D,L-lactic-co-glycolic acid) (PLGA), and combinations thereof. In some embodiments, the non-cellular delivery vehicle comprises agarose, latex, or polystyrene. One or more of the polypeptides described herein can be conjugated to a non-cellular delivery vehicle using standard methods known in the art (see, e.g., Ulbrich et al. (2016) Chem Rev. 116(9): 5338-431). Conjugation can be either covalent or non-covalent. For example, in embodiments in which the non-cellular delivery vehicle is a liposome, a polypeptide described herein may be attached to the liposome via a polyethylene glycol (PEG) chain. Conjugation of a polypeptide to a liposome can also involve thioester bonds, for example by reaction of thiols and maleimide groups. Cross-linking agents can be used to create sulfhydryl groups for attachment of polypeptides to non-cellular delivery vehicles (see, e.g., Paszko and Senge (2012) Curr. Med. Chem. 19(31): 5239-77). In some embodiments, the non-cellular delivery vehicles comprising one or more of the polypeptides described herein may be used in any of therapeutic methods provided herein.

### IV. Methods of Using Artificial Antigen Presenting Cells

The present disclosure contemplates various methods of using the aAPCs described herein. As would be understood by one skilled in the art, based upon the disclosure provided herein, the dose and timing of administration of the aAPCs can be specifically tailored for each application described herein. More specifically, where it is desirable to provide stimulation to a T cell using certain molecules expressed by an aAPC, or several aAPCs, followed by stimulation using another aAPC, or several aAPCs, expressing a different, even if overlapping, set of molecules, then a combination of cis and trans approaches can be utilized. In essence, the aAPCs of the disclosure, and the methods disclosed herein, provide an almost limitless number of variations and the disclosure is not limited in any way to any particular combination or approach. The skilled artisan, armed with the teachings provided herein and the knowledge available in the art, can readily determine the desired approach for each particular T cell.

In one aspect, the disclosure features a method of activating an antigen-specific T cell, the method comprising contacting the T cell with an aAPC as described herein, thereby activating the antigen-specific T cell. In some embodiments, activating the antigen-specific T cell includes activating an antigen-specific T cell with a central memory phenotype. In certain embodiments, activating an antigen-specific T cell includes promoting the expansion of memory T cells. In other embodiments, activating an antigen-specific T cell includes differentiating or de-differentiating T cells. For example, long-lived memory CD8 T cells are derived from a subset of effector T cells through a process of de-differentiation.

In another aspect, the disclosure features a method of suppressing activity of a T cell, the method comprising contacting the T cell with an aAPC engineered to suppress T cell activity, thereby suppressing activity of the T cell.

In another aspect, the invention features a method for activating a Treg cell, the method comprising contacting the Treg cell with an aAPC engineered to activate a regulatory T cell (Treg), thereby activating the Treg cell.

In another aspect, the disclosure features a method for inducing proliferation of a T cell expressing a receptor molecule, the method comprising contacting the T cell with an aAPC as described herein, wherein the costimulatory polypeptide specifically binds with the receptor molecule, thereby inducing proliferation of said T cell.

The disclosure also encompasses a method for specifically inducing proliferation of a T cell expressing a known co-stimulatory molecule. The method comprises contacting a population of T cells comprising at least one T cell expressing the known co-stimulatory molecule with an aAPC engineered to present a ligand of the co-stimulatory molecule. As disclosed elsewhere herein, where an aAPC expresses at least one co-stimulatory ligand that specifically binds with a co-stimulatory molecule on a T cell, binding of the co-stimulatory molecule with its cognate co-stimulatory ligand induces proliferation of the T cell. Thus, the T cell of interest is induced to proliferate without having to first purify the cell from the population of cells. Also, this method provides a rapid assay for determining whether any cells in the population are expressing a particular costimulatory molecule of interest, since contacting the cells with the aAPC will induce proliferation and detection of the growing cells thereby identifying that a T cell expressing a costimulatory molecule of interest was present in the sample. In this way, any T cell of interest where at least one costimulatory molecule on the surface of the cell is known, can be expanded and isolated.

The disclosure also includes a method for specifically expanding a T cell population subset. More particularly, the method comprises contacting a population of T cells comprising at least one T cell of a subset of interest with an aAPC capable of expanding that T cell, or at least an aAPC expressing at least one costimulatory ligand that specifically binds with a cognate costimulatory molecule on the surface of the T cell. Binding of the co-stimulatory molecule with its binding partner co-stimulatory ligand induces proliferation of the T cell, thereby specifically expanding a T cell population subset. One skilled in the art would understand, based upon the disclosure provided herein, that T cell subsets include T helper (T_{H1} and T_{H2}) CD4 expressing, cytotoxic T lymphocyte (CTL) (Tc1 or Tc2) T regulatory (TR_{EG}), T_{C/S}, naive, memory, central memory, effector memory, and γΔT cells. Therefore, cell populations enriched for a particular T cell subset can be readily produced using the method of the disclosure.

In certain embodiments, T cells are expanded to between about 100 and about 1,000,000 fold, or between about 1,000 and about 1,000,000 fold, e.g., between 1,000 and about 100,000 fold.

The fitness of T cells after ex vivo expansion is an excellent predicator of their ability to function in vivo. Thus, in certain embodiments, the ability of the aAPCs to induce the key cell survival gene Bcl-xL is also measured. The percentage of apoptotic cells in a culture during the expansion process is used to determine whether any of the cell based aAPCs confer a particular survival advantage to the expanded T cells. Additionally, the telomere length of cells after ex vivo expansion can be measured to determine if a particular aAPC is more effective in preserving the replicative potential of the cells it expands.

The disclosure also includes a method for identifying a co-stimulatory ligand, or combination thereof, which specifically induces activation of a T cell subset. Briefly, the method comprises contacting a population of T cells with an aAPC presenting (e.g. comprising on the cell surface) at least one co-stimulatory ligand, and comparing the level of proliferation of the T cell subset contacted with the aAPC with the level of proliferation of an otherwise identical T cell subset not contacted with the aAPC. A greater level of proliferation of the T cell subset contacted with the aAPC compared with the level of proliferation of the otherwise identical T cell subset which was not contacted with the aAPC is an indication that at the co-stimulatory ligand specifically induces activation of the T cell subset to which that T cell belongs.

The method permits the identification of a costimulatory ligand that specifically expands a T cell subset where it is not previously known which factor(s) expand that T cell subset. The skilled artisan would appreciate that in order to minimize the number of screenings, it is preferable to transduce as many nucleic acids encoding costimulatory ligands such that the number of assays can be reduced. Further, the method allows, by combining the various proteins (e.g., stimulatory ligand, costimulatory ligand, antigen, cytokine, and the like), to assess which combination(s) of factors will make the most effective aAPC, or combination of aAPCs, to expand the T cell subset. In this way, the various requirements for growth and activation for each T cell subset can be examined. Further, to evaluate T cell expansion, CFSE staining can be used. aAPCs are mixed with CD8 T cells (e.g. from a subject suffering from a disease or disorder, such an autoimmune disease, an infectious disease or cancer). To compare the initial rate of cell expansion, the cells are subject to CFSE staining to determine how well each aAPC induced the proliferation of all T cells. CFSE staining provides a much more quantitative endpoint and allows simultaneous phenotyping of the expanded cells. Every day after stimulation, an aliquot of cells is removed from each culture and analyzed by flow cytometry. CFSE staining makes cells highly fluorescent. Upon cell division, the fluorescence is halved and thus the more times a cell divides the less fluorescent it becomes. The ability of each aAPC to induce T cell proliferation is quantitated by measuring the number of cells that divided once, twice, three times and so on. The aAPC that induces the most number of cell divisions at a particular time point is deemed as the most potent expander.

To determine how well these aAPCs promote long-term growth of T cells, cell growth curves are generated. These experiments are set up exactly as the CFSE experiments, but no CFSE is used. Every 2-3 days of culture, T cells are removed from the respective cultures and counted using a Coulter counter which measures how many cells are present and the mean volume of the cells. The mean cell volume is the best predicator of when to restimulate the cells. In general, when T cells are properly stimulated they triple their cell volume. When this volume is reduced to more than about half of the initial blast, it may be necessary to restimulate the T cells to maintain a log linear expansion (Levine et al., 1996, Science 272:1939-1943; Levine et al., 1997, J. Immunol. 159:5921-5930). The time it takes each aAPC to induce 20 population doublings is calculated. The relative differences of each aAPC to induce this level of T cell expansion is an important criteria on which a particular aAPC is used to move forward to clinical trials.

The phenotypes of the cells expanded by each aAPC are characterized to determine whether a particular subset is preferentially expanded. Prior to each restimulation, a phenotype analysis of the expanding T cell populations is performed to define the differentiation state of the expanded T cells using the CD27 and CD28 definitions proposed by Appay et al. (2002, Nature Med. 8, 379-385) and CCR7 definitions proposed by Sallusto et al. (1999, Nature 401:708-712). Perforin and Granzyme B intracellular staining are used to perform a gross measure to estimate cytolytic potential.

In one aspect, the method comprises contacting various aAPCs with the T cell subset without first characterizing the costimulatory molecules on the surface of the T cell subset. Also, the disclosure encompasses a method where the costimulatory molecule(s) present on the surface of the T cell subset are examined prior to contacting the aAPCs with the cell. Thus, the present disclosure provides a novel assay for determining the growth requirements for various T cell subsets.

In another aspect, the disclosure features a method of treating a subject in need of an altered immune response, the method comprising contacting T cells of the subject with an APC as described herein, thereby treating the subject in need of an altered immune response. In some embodiments, the contacting is in vitro or in vivo. In some embodiments, the contacting is in vitro. In some embodiments, the contacting is in vivo.

In another aspect, the disclosure features a method of treating a subject in need of an altered immune response, the method comprising a) determining an HLA status of the subject, b) selecting an artificial antigen presenting cell (aAPC) that is immunologically compatible with the subject, wherein the aAPC is an engineered erythroid cell expressing a first exogenous antigenic polypeptide, and c) administering the aAPC to the subject, thereby treating the subject in need of the altered immune response.

In another aspect, the disclosure features a method of treating a subject in need of an altered immune response, the method comprising a) determining an expression profile of an antigen in the subject, b) selecting an artificial antigen presenting cell (aAPC), wherein the aAPC is an engineered erythroid cell expressing a first exogenous antigenic polypeptide, and c) administering the aAPC to the subject, thereby treating the subject in need of the altered immune response.

The immune response induced in the subject by administering the aAPCs of the present disclosure may include cellular immune responses mediated by CD8⁺ T cells, capable of killing tumor and infected cells, and CD4⁺ T cell responses. Humoral immune responses, mediated primarily by B cells that produce antibodies following activation by CD4⁺ T cells, may also be induced. A variety of techniques may be used for analyzing the type of immune responses induced by the compositions of the present disclosure, which are well described in the art; e.g., Coligan et al., Current Protocols in Immunology, John Wiley & Sons Inc., 1994.

CD8+ cytotoxic T cells respond to antigens in association with MHC I molecules. CD4+ helper T cells respond to antigens in association with MHC II molecules. Activated CD4+ T cells in turn activate new and existing CD8+ T cells (both antigen specific and naïve CD8+ T cells). Accordingly, it is contemplated in some embodiments of the invention that an aAPC that expands and activates antigen specific CD8+ T cells (an aAPC comprising an antigen in association with MHC I) can be administered with an aAPC that activates CD4+ T cells (an aAPC comprising an antigen in association with MHC II), thereby potentiating the immune response. In some embodiments, an aAPC that expands and activates antigen specific CD8+ T cells can be administered with an aAPC that activates CD4+ T cells, thereby synergistically increasing the robustness of the immune response. In some embodiments, an aAPC comprising an antigen in association with MHC I and a costimulatory polypeptide can be administered with an aAPC comprising an antigen in association with MHC II. In other embodiments, an aAPC comprising an antigen in association with MHC I can be administered with an aAPC comprising an antigen in association with MHC II and a costimulatory polypeptide. In some embodiments, an aAPC comprising an antigen in association with MHC I and a costimulatory polypeptide can be administered with an aAPC comprising an antigen in association with MHC II and a costimulatory polypeptide.

In some embodiments, an aAPC that comprises an exogenous antigen-presenting polypeptide and an exogenous antigenic polypeptide, wherein the exogenous antigen-presenting polypeptide is an MHC class I polypeptide or an MHC class I single chain fusion, together with an aAPC that comprises an exogenous antigen-presenting polypeptide and an exogenous antigenic polypeptide, wherein the exogenous antigen-presenting polypeptide is an MHC class II polypeptide or an MHC class II single chain fusion, is administered to a subject. Administering an antigen in association with MHC I, in combination with an antigen in association with MHC II, provides complementary roles of CD8+ and CD4+ T cells.

In further aspects the present disclosure also provides methods for expanding a population of T cells by cell surface moiety ligation. In addition, it is an object to provide a method of inducing a population of T cells from a subject to rapidly proliferate exponentially for a long term to sufficient numbers for research purposes, comprising isolating a population of T cells from a subject, activating the population of T cells by contacting the T cells ex vivo with at least one exogenous polypeptide that provides a primary activation signal to the T cells; and stimulating the activated T cells with at least one second exogenous polypeptide that provides a co-stimulatory signal, such that T cells that have received a primary activation signal are stimulated to rapidly proliferate. In particular, it is an object to provide such a method when the subject is human, and wherein the method further comprises using the activated T cells to identify antigens in the subject. Moreover, when the subject is infected with a disease or condition, having at least one antigen related thereto, the provided method further comprises using the activated T cells to identify the at least one antigen. The antigen may comprise, e.g., and without limitation, a tumor antigen, an antigen relating to an autoimmune disorder or condition, or an infectious disease or pathogen. The method further comprises screening the at least one antigen as a target molecule for research purposes, or for developing a vaccine based upon the at least one antigen.

Treatment of Conditions that would Benefit from Modulation of T Cell Response

Methods of administering engineered erythroid cells comprising (e.g., presenting) exogenous agent (e.g., polypeptides) are described, e.g., in WO2015/073587 and WO2015/153102, each of which is incorporated by reference in its entirety.

In embodiments, the aAPCs comprising engineered erythroid cells described herein are administered to a subject, e.g., a mammal, e.g., a human. Exemplary mammals that can be treated include without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like). The methods described herein are applicable to both human therapy and veterinary applications. In some embodiments, the erythroid cell is an enucleated cell. In some embodiments, the erythroid cell is a nucleated cell.

In some embodiments, the erythroid cells are administered to a patient every 1, 2, 3, 4, 5, or 6 months.

In some embodiments, a dose of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some embodiments, the erythroid cells are administered to a patient in a dosing regimen (dose and periodicity of administration) sufficient to maintain function of the administered erythroid cells in the bloodstream of the patient over a period of 2 weeks to a year, e.g., one month to one year or longer, e.g., at least 2 weeks, 4 weeks, 6 weeks, 8 weeks, 3 months, 6 months, a year, 2 years.

In some embodiments, the erythroid cells (e.g. aAPCs as described herein) are administered to a patient in two doses or more (e.g. 2, 3, 4 or more doses). In further embodiments, the erythroid cells (e.g. aAPCs as described herein) are administered to a patient in two doses or more, wherein the second dose is administered at a time after the first dose when T-cell proliferation is determined to be at a peak. In some embodiments, the aAPCs of the disclosure are administered in a first dose, wherein the first dose stimulates T cell proliferation and activation. Following the first dose, the aAPCs of the disclosure are administered in a second dose, when T cells are activated and proliferation is at its peak, to stimulate T cell expansion. Without being bound by theory, administering the aAPCs of the disclosure in two doses or more, increases the capacity of the aAPCs to boost the memory T cell population and thereby provide longer efficacy, e.g., efficacy against a relapse of a tumor or re-challenge with an infectious agent.

Peak T-cell proliferation can be determined using methods known to the skilled artisan. For example, peak T-cell proliferation can be determined by 3H-thymidine incorporation by proliferating T-cells, or by labelling proliferating T-cells with the fluorescent dye 5,6-carboxyfluorescein diacetate succinimidyl ester (CFSE).

In some aspects, the present disclosure provides a method of treating a disease or condition described herein, comprising administering to a subject in need thereof a composition described herein, e.g., an aAPC described herein. In some embodiments, the disease or condition is a cancer. In some embodiments, the disease or condition is an autoimmune disease. In some embodiments, the disease or condition is an autoimmune disease associated with or triggered by an infectious agent. In some embodiments, the disease or condition is an infectious disease.

In some aspects, the disclosure provides a use of an aAPC described herein for treating a disease or condition described herein, e.g., cancer, an autoimmune disease, such as an autoimmune disease triggered by an infectious agent, or an infectious disease. In some aspects, the disclosure provides a use of an aAPC described herein for manufacture of a medicament for treating a disease or condition described herein, e.g., cancer, an autoimmune disease, such as an autoimmune disease triggered by an infectious agent, or an infectious disease.

In some embodiments, the aAPC comprises a first exogenous polypeptide comprising a first therapeutic (e.g. an anti-cancer therapeutic, an autoimmune therapeutic, an infectious disease therapeutic). In certain preferred embodiments, the first therapeutic is an antigen, e.g., a tumor antigen, an antigen relating to an autoimmune disorder or condition, such as an autoimmune disease triggered by an infectious agent, or to an infectious disease or pathogen. In embodiments, the aAPC further comprises a second exogenous polypeptide wherein the second exogenous polypeptide comprises an antigen-presenting polypeptide, e.g., MHCI or MHCII. In embodiments, the aAPC further comprises a third exogenous polypeptide, wherein the third exogenous polypeptide comprises at least one costimulatory polypeptide, coinhibitory polypeptide, or Treg expansion polypeptide as disclosed herein.

In some embodiments, the aAPC comprises a first exogenous polypeptide comprising a first therapeutic (e.g. an anti-cancer therapeutic, an autoimmune therapeutic, an infectious disease therapeutic) and a second exogenous polypeptide, comprising a second therapeutic (e.g. an anti-cancer therapeutic, an autoimmune therapeutic, an infectious disease therapeutic). In certain preferred embodiments, therapeutic is an antigenic polypeptide, e.g., a tumor antigen, an antigen relating to an autoimmune disorder or condition, such as an autoimmune disease triggered by an infectious agent, or to an infectious disease or pathogen. In embodiments, the aAPC further comprises a third exogenous polypeptide wherein the third exogenous polypeptide comprises an antigen-presenting polypeptide, e.g., MHCI or MHCII. In embodiments, the aAPC further comprises a fourth exogenous polypeptide, wherein the fourth exogenous polypeptide comprises at least one costimulatory polypeptide, coinhibitory polypeptide, or Treg expansion polypeptide as disclosed herein.

In some embodiments, the aAPC comprises a first exogenous polypeptide comprising a first therapeutic (e.g. an anti-cancer therapeutic, an autoimmune therapeutic, an infectious disease therapeutic), a second exogenous polypeptide, comprising a second therapeutic (e.g. an anti-cancer therapeutic, an autoimmune therapeutic, an infectious disease therapeutic), and a third exogenous polypeptide, comprising a third therapeutic (e.g. an anti-cancer therapeutic, an autoimmune therapeutic, an infectious disease therapeutic). In certain preferred embodiments, therapeutic is an antigenic polypeptide, e.g., a tumor antigen, an antigen relating to an autoimmune disorder or condition, or to an infectious disease or pathogen. In embodiments, the aAPC further comprises a fourth exogenous polypeptide wherein the fourth exogenous polypeptide comprises an antigen-presenting polypeptide, e.g., MHCI or MHCII. In embodiments, the aAPC further comprises a fifth exogenous polypeptide, wherein the third exogenous polypeptide comprises at least one costimulatory polypeptide, coinhibitory polypeptide, or Treg expansion polypeptide as disclosed herein.

In embodiments, any two or more of the first, second and third therapeutic can recognize, bind and/or act on the same target, for example a cell surface receptor and/or an endogenous human protein. Alternatively, the first, second and third therapeutic can act on different targets.

The first, second or third targets may be members of the same biological pathway, wherein optionally the targets are cell surface receptors, endogenous human proteins. As used herein, the term “pathway” or “biological pathway” refers to a plurality of biological molecules, e.g., polypeptides, that act together in a sequential manner. Examples of pathways include signal transduction cascades and complement cascades. In some embodiments, a pathway begins with detection of an extracellular signal and ends with a change in transcription of a target gene. In some embodiments, a pathway begins with detection of a cytoplasmic signal and ends with a change in transcription of a target gene. A pathway can be linear or branched. If branched, it can have a plurality of inputs (converging), or a plurality of outputs (diverging).

The first, second or third targets may be on the same cell types or on different cell types. In some embodiments, the first exogenous polypeptide binds a first cell, e.g., a first cell type, and the second exogenous polypeptide binds a second cell, e.g., a second cell type. In some embodiments, the first and second cell types are the same. In some embodiments, the first and second cell types are different. In some embodiments, the first exogenous polypeptide localizes the engineered erythroid cell to a desired site, e.g., a human cell, and the second exogenous polypeptide has a therapeutic activity, e.g., antigen presenting activity.

In certain embodiments, therapeutic is a suitable exogenous antigen that is chosen to interact with a specific target. Suitable targets include entities that are associated with a specific disease, disorder, or condition (e.g. cancer, autoimmune disease, infectious disease). However, targets may also be chosen independent of a specific disease, disorder, or condition.

In some embodiments, the effect of the first and second, or first and second and third exogenous polypeptide is synergistic. The term “synergistic” or “synergy” means a more than additive effect of a combination of two or more agents (e.g., polypeptides that are part of an engineered erythroid cell) compared to their individual effects. In certain embodiments, synergistic activity is a more-than-additive effect of an engineered erythroid cell comprising a first polypeptide and a second polypeptide, compared to the effect of an engineered erythroid cell comprising the first polypeptide and an engineered erythroid cell comprising the second polypeptide. In some embodiments, synergistic activity is present when a first agent produces a detectable level of an output X, a second agent produces a detectable level of the output X, and the first and second agents together produce a more-than-additive level of the output X.

Cancer

In certain embodiments, the present disclosure provides an aAPC comprising an erythroid cell (e.g. an enucleated erythroid cell) or an enucleated cell, comprising one or more exogenous polypeptides, wherein one of the one or more exogenous polypeptides comprises a therapeutic for the treatment of cancer (an anti-cancer therapeutic). In some embodiments, the aAPC comprises a first exogenous polypeptide comprising a first anti-cancer therapeutic and a second exogenous polypeptide, comprising a second anti-cancer therapeutic. In some embodiments, the aAPC comprises a first exogenous polypeptide comprising a first anti-cancer therapeutic, a second exogenous polypeptide, comprising a second anti-cancer therapeutic, and a third exogenous polypeptide, comprising a third anti-cancer therapeutic. Any two or more of the first, second and third anti-cancer therapeutic can act on the same target, for example a cell surface receptor and/or an endogenous human protein. Alternatively, the first, second and third anti-cancer therapeutic can act on different targets. Any two or more of the first, second and third targets may be members of the same biological pathway, wherein optionally the targets are cell surface receptors, endogenous human proteins. The first, second or third targets may be on different cell types. In some embodiments, the first exogenous polypeptide localizes the engineered erythroid cell to a desired site, e.g., a human cell, and the second exogenous polypeptide has a therapeutic activity, e.g., antigen presenting activity.

In certain preferred embodiments, the first therapeutic is an antigen, e.g., a tumor antigen. In certain embodiments, the first therapeutic and second therapeutic is an antigen, e.g., a tumor antigen. In certain embodiments, the first, second and third therapeutic is an antigen, e.g., a tumor antigen.

In embodiments, the aAPC further comprises an additional exogenous polypeptide wherein the additional exogenous polypeptide comprises an antigen-presenting polypeptide, e.g., an MHC molecule (e.g. MHCI or MHCII). In embodiments, the aAPC further comprises an additional exogenous polypeptide, wherein the additional exogenous polypeptide comprises at least one costimulatory polypeptide. In some embodiments, the aAPC further comprises an additional exogenous polypeptide comprising an antigen-presenting polypeptide and an additional exogenous polypeptide comprising at least one costimulatory polypeptide.

In some embodiments, the aAPC comprises a first exogenous polypeptide comprising an antigen-presenting polypeptide, e.g., an MHC molecule (e.g. MHCI or MHCII), and the second exogenous polypeptide comprises an antigen (e.g., tumor associated antigen).

It is contemplated in some embodiments of the invention that an aAPC that expands and activates antigen specific CD8+ T cells in the tumor (an aAPC comprising an antigen in association with MHC I) can be administered with an aAPC that activates CD4+ T cells (an aAPC comprising an antigen in association with MHC II), which activates both antigen-specific and naïve CD8+ T-cells in the tumor and lymph nodes, thereby potentiating a robust anti-tumor response. In some embodiments, an aAPC that expands and activates antigen specific CD8+ T cells in the tumor can be administered with an aAPC that activates CD4+ T cells, which activates both antigen-specific and naïve CD8+ T-cells in the tumor and lymph nodes, thereby synergistically increasing the robustness of the immune response.

In some embodiments, an aAPC that comprises a first exogenous antigen-presenting polypeptide and a second exogenous polypeptide comprising an antigen (e.g. a first tumor associated antigen), wherein the exogenous antigen-presenting polypeptide is an MHC class I polypeptide or an MHC class I single chain fusion is administered to a subject together with an aAPC that comprises a first exogenous antigen-presenting polypeptide and a second exogenous polypeptide comprising an antigen (e.g. a second tumor associated antigen), wherein the exogenous antigen-presenting polypeptide is an MHC class II polypeptide or an MHC class II single chain fusion, is administered to a subject. In some embodiments, an aAPC comprising an antigen in association with MHC I and a costimulatory polypeptide can be administered with an aAPC comprising an antigen in association with MHC II. In other embodiments, an aAPC comprising an antigen in association with MHC I can be administered with an aAPC comprising an antigen in association with MHC II and a costimulatory polypeptide. In some embodiments, an aAPC comprising an antigen in association with MHC I and a costimulatory polypeptide can be administered with an aAPC comprising an antigen in association with MHC II and a costimulatory polypeptide.

Without being bound by theory, it is thought that MHC I and MHC II tumor antigen presentation, combined with potent co-stimulation, has the potential to generate sustained tumor-specific killing.

It is encompassed by the present invention that the antigen may be any tumor antigen, or antigenic-portion thereof, known in the art, including, without limitation, any one or more of the antigens, or antigenic-portions thereof, presented in Table 1. It will be recognized by the skilled artisan that an aAPC comprising an antigen presented in Table 1 can be used to treat a cancer, for example, the specific cancer corresponding to the particular antigen or antigenic peptide as presented in Table 1.

Accordingly, in some aspects, the disclosure provides a method of treating a subject having cancer, comprising administering to the subject an effective number of the aAPCs described herein to the subject, thereby treating the cancer.

In another aspect, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a tumor associated antigen, thereby treating the cancer. Any combination of particular cancer, as the cancer to be treated in the method, and tumor associated antigen, as antigen to be present on the aAPC, as presented together in Table 1, is contemplated by the present invention.

In another aspect, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A antigen, thereby treating the cancer. In some embodiments, the erythroid cell is an enucleated cell. In some embodiments, the erythroid cell is a nucleated cell. In some embodiments, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A1 antigen, thereby treating the cancer. In some embodiments, the MAGE-A1 antigen is a MAGE-A1 antigen presented in Table 1. In some embodiments, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A2 antigen, thereby treating the cancer. In some embodiments, the MAGE-A2 antigen is a MAGE-A2 antigen presented in Table 1. In some embodiments, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A3 antigen, thereby treating the cancer. In some embodiments, the MAGE-A3 antigen is a MAGE-A3 antigen presented in Table 1. In some embodiments, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A4 antigen, thereby treating the cancer. In some embodiments, the MAGE-A4 antigen is a MAGE-A4 antigen presented in Table 1. In some embodiments, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A5 antigen, thereby treating the cancer. In some embodiments, the MAGE-A5 antigen is a MAGE-A5 antigen presented in Table 1. In some embodiments, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A6 antigen, thereby treating the cancer. In some embodiments, the MAGE-A6 antigen is a MAGE-A6 antigen presented in Table 1. In some embodiments, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A7 antigen, thereby treating the cancer. In some embodiments, the MAGE-A7 antigen is a MAGE-A7 antigen presented in Table 1. In some embodiments, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A8 antigen, thereby treating the cancer. In some embodiments, the MAGE-A8 antigen is a MAGE-A8 antigen presented in Table 1. In some embodiments, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A9 antigen, thereby treating the cancer. In some embodiments, the MAGE-A9 antigen is a MAGE-A9 antigen presented in Table 1. In some embodiments, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A10 antigen, thereby treating the cancer. In some embodiments, the MAGE-A10 antigen is a MAGE-A10 antigen presented in Table 1. In some embodiments, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A11 antigen, thereby treating the cancer. In some embodiments, the MAGE-A11 antigen is a MAGE-A11 antigen presented in Table 1. In some embodiments, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises a MAGE-A12 antigen, thereby treating the cancer. In some embodiments, the MAGE-A12 antigen is a MAGE-A1 antigen presented in Table 1. In some embodiments, the MAGE-A2 antigen is a MAGE-A12 antigen presented in Table 1.

In some embodiments, the cancer is acute myelogenous leukemia (AML). In some embodiments, the cancer is melanoma. In some embodiments, the cancer is a solid tumor.

In another aspect, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises an immunogenic peptide of MAGE-A antigen. In some embodiments, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises an epitope common to several tumor antigens of the MAGE-A family. In some embodiments, the exogenous antigenic polypeptide is p248v9, an immunogenic peptide presented by HLA-A*0201 and capable of inducing cytotoxic T lymphocytes which recognize all the MAGE-A antigens. In some embodiments, the exogenous antigenic polypeptide is the immunogenic peptide p248g9 (YLEYRQVPG (SEQ ID NO: 156)). In some embodiments, the exogenous antigenic polypeptide is the immunogenic peptide p248d9 (YLEYRQVPD (SEQ ID NO: 125)).

In some embodiments, the cancer is acute myelogenous leukemia (AML). In some embodiments, the cancer is melanoma. In some embodiments, the cancer is a solid tumor.

In another aspect, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous antigenic polypeptides comprises a neutrophil granule protease antigen, thereby treating the cancer. In some embodiments, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises a neutrophil elastase antigen, thereby treating the cancer. In some embodiments, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises a proteinase 3 antigen, thereby treating the cancer. In some embodiments, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises a cathepsin G antigen, thereby treating the cancer.

In some embodiments, the cancer is acute myelogenous leukemia (AML). In some embodiments, the cancer is chronic myelogenous leukemia (CML). In some embodiments, the cancer is breast cancer. In some embodiments, the cancer is lung cancer. In some embodiments, the cancer is melanoma. In some embodiments, the cancer is a solid tumor.

In another aspect, the present disclosure provides a method of treating a subject having cancer, comprising administering to the subject an effective number of aAPCs comprising an erythroid cell (e.g. an enucleated erythroid cell), comprising one or more exogenous polypeptides, wherein one of the one or more exogenous polypeptides comprises PR1 (VLQELNVTV (SEQ ID NO: 225)).

In some embodiments, the cancer is acute myelogenous leukemia (AML). In some embodiments, the cancer is chronic myelogenous leukemia (CML). In some embodiments, the cancer is breast cancer. In some embodiments, the cancer is lung cancer. In some embodiments, the cancer is melanoma. In some embodiments, the cancer is a solid tumor.

In another aspect, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises a NY-ESO-1/LAGE-2 antigen, thereby treating the cancer.

In some embodiments, the cancer is multiple myeloma. In some embodiments, the cancer is non-small cell lung cancer. In some embodiments, the cancer is ovarian cancer. In some embodiments, the cancer is melanoma. In some embodiments, the cancer is a solid tumor.

In another aspect, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises at least one NY-ESO-1/LAGE-2 derived peptide. In some embodiments, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprise at least one exogenous HLA class I-binding polypeptide derived from NY-ESO-1/LAGE-2. In some embodiments, the polypeptide is SLLMWITQC (SEQ ID NO: 110). In some embodiments, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprise at least one exogenous HLA class II-binding polypeptide derived from NY-ESO-1/LAGE-2. In some embodiments, the polypeptide is SLLMWITQCFLPVF (SEQ ID NO: 114).

In some embodiments, the exogenous antigenic polypeptide comprises a NY-ESO-1/LAGE-2 antigen selected from SLLMWITQC (SEQ ID NO: 110), MLMAQEALAFL (SEQ ID NO: 109), YLAMPFATPME (SEQ ID NO: 204), ASGPGGGAPR (SEQ ID NO: 205), LAAQERRVPR (SEQ ID NO: 111), TVSGNILTIR (SEQ ID NO: 206), APRGPHGGAASGL (SEQ ID NO: 207), MPFATPMEAEL (SEQ ID NO: 208), KEFTVSGNILTI (SEQ ID NO: 209), MPFATPMEA (SEQ ID NO: 210), FATPMEAEL (SEQ ID NO: 211), FATPMEAELAR (SEQ ID NO: 212), LAMPFATPM (SEQ ID NO: 213), ARGPESRLL (SEQ ID NO: 214), SLLMWITQCFLPVF (SEQ ID NO: 114), LLEFYLAMPFATPMEAELARRSLAQ (SEQ ID NO: 215), LLEFYLAMPFATPMEAELARRSLAQ (SEQ ID NO: 215), EFYLAMPFATPM (SEQ ID NO: 216), PGVLLKEFTVSGNILTIRLTAADHR (SEQ ID NO: 217), RLLEFYLAMPFA (SEQ ID NO: 218), QGAMLAAQERRVPRAAEVPR (SEQ ID NO: 115), PFATPMEAELARR (SEQ ID NO: 219), PGVLLKEFTVSGNILTIRLT (SEQ ID NO: 220), VLLKEFTVSG (SEQ ID NO: 221), AADHRQLQLSISSCLQQL (SEQ ID NO: 116), LLEFYLAMPFATPMEAELARRSLAQ (SEQ ID NO: 215), LKEFTVSGNILTIRL (SEQ ID NO: 222), PGVLLKEFTVSGNILTIRLTAADHR (SEQ ID NO: 217), LLEFYLAMPFATPMEAELARRSLAQ (SEQ ID NO: 215), KEFTVSGNILT (SEQ ID NO: 223), LLEFYLAMPFATPM (SEQ ID NO: 224), and AGATGGRGPRGAGA (SEQ ID NO: 119). In some embodiments, the NY-ESO-1/LAGE-2 antigen is one or more NY-ESO-1/LAGE-2 antigens presented in Table 1.

In some embodiments, the cancer is multiple myeloma. In some embodiments, the cancer is non-small cell lung cancer. In some embodiments, the cancer is ovarian cancer. In some embodiments, the cancer is melanoma. In some embodiments, the cancer is a solid tumor.

In another aspect, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides is a gp100 polypeptide. In some embodiments, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides is a gp100 polypeptide, and wherein the erythroid cell further presents, e.g. comprises on the cell surface, an exogenous polypeptide comprising a costimulatory polypeptide. In some embodiments, the costimulatory polypeptide is 4-1BBL. In some embodiments, the present disclosure provides a method of treating a subject having cancer, comprising administering to the subject an effective number of aAPCs comprising an erythroid cell (e.g. an enucleated erythroid cell), comprising an exogenous antigen-presenting polypeptide and an exogenous antigenic polypeptide, wherein the exogenous antigenic polypeptide is specifically bound to the exogenous antigen-presenting polypeptide (e.g. an MHC class I or class II molecule), wherein the exogenous antigen-presenting polypeptide is an MHC class I polypeptide or single chain fusion or an MHC class II polypeptide or single chain fusion, wherein the exogenous antigenic polypeptide is a gp100 antigen. In some embodiments, the present disclosure provides a method of treating a subject having cancer, comprising administering to the subject an effective number of aAPCs comprising an erythroid cell (e.g. an enucleated erythroid cell), comprising an exogenous antigen-presenting polypeptide and an exogenous antigenic polypeptide, wherein the exogenous antigenic polypeptide is specifically bound to the exogenous antigen-presenting polypeptide (e.g. an MHC class I or class II molecule), wherein the exogenous antigen-presenting polypeptide is an MHC class I polypeptide or single chain fusion or an MHC class II polypeptide or single chain fusion, wherein the exogenous antigenic polypeptide is a gp100 antigen, and wherein the erythroid cell further presents, e.g. comprises on the cell surface, an exogenous polypeptide comprising a costimulatory polypeptide. In some embodiments, the costimulatory polypeptide is 4-1BBL. In some embodiments, the present disclosure provides a method of treating a subject having cancer, comprising administering to the subject an effective number of aAPCs comprising an erythroid cell (e.g. an enucleated erythroid cell), comprising an exogenous antigen-presenting polypeptide and an exogenous antigenic polypeptide, wherein the exogenous antigenic polypeptide is specifically bound to the exogenous antigen-presenting polypeptide (e.g. an MHC class I or class II molecule), wherein the exogenous antigen-presenting polypeptide is an MHC class I polypeptide or single chain fusion, wherein the exogenous antigenic polypeptide is a gp100 antigen. In some embodiments, the exogenous antigen-presenting polypeptide is MHCI-2Db. In some embodiments, the present disclosure provides a method of treating a subject having cancer, comprising administering to the subject an effective number of aAPCs comprising an erythroid cell (e.g. an enucleated erythroid cell), comprising an exogenous antigen-presenting polypeptide and an exogenous antigenic polypeptide, wherein the exogenous antigen-presenting polypeptide is an MHC class I polypeptide or single chain fusion, wherein the exogenous antigenic polypeptide is a gp100 antigen, and wherein the erythroid cell further presents, e.g. comprises on the cell surface, an exogenous polypeptide comprising a costimulatory polypeptide. In some embodiments, the costimulatory polypeptide is 4-1BBL. In some embodiments, the exogenous antigen-presenting polypeptide is MHCI-2Db.

In some embodiments, the exogenous antigenic polypeptide comprises a gp100 antigen selected from KTWGQYWQV (SEQ ID NO: 306), (A)MLGTHTMEV (SEQ ID NO: 307), IMDQVPFSV (SEQ ID NO: 308), ITDQVPFSV (SEQ ID NO: 309), YLEPGPVTA (SEQ ID NO: 310), LLDGTATLRL (SEQ ID NO: 311), VLYRYGSFSV (SEQ ID NO: 312), SLADTNSLAV (SEQ ID NO: 313), RLMKQDFSV (SEQ ID NO: 314), RLPRIFCSC (SEQ ID NO: 315), LIYRRRLMK (SEQ ID NO: 316), ALLAVGATK (SEQ ID NO: 317), IALNFPGSQK (SEQ ID NO: 318), RSYVPLAHR (SEQ ID NO: 319), ALNFPGSQK (SEQ ID NO: 320), ALNFPGSQK (SEQ ID NO: 320), VYFFLPDHL (SEQ ID NO: 321), RTKQLYPEW (SEQ ID NO: 322), HTMEVTVYHR (SEQ ID NO: 323), SSPGCQPPA (SEQ ID NO: 324), VPLDCVLYRY (SEQ ID NO: 325), LPHSSSHWL (SEQ ID NO: 326), SNDGPTLI (SEQ ID NO: 327), GRAMLGTHTMEVTVY (SEQ ID NO: 328), WNRQLYPEWTEAQRLD (SEQ ID NO: 329), TTEWVETTARELPIPEPE (SEQ ID NO: 330), TGRAMLGTHTMEVTVYH (SEQ ID NO: 331), GRAMLGTHTMEVTVY (SEQ ID NO: 328). In some embodiments, the gp100 antigen is one or more gp100 antigens presented in Table 1.

In some embodiments, the cancer is multiple myeloma. In some embodiments, the cancer is non-small cell lung cancer. In some embodiments, the cancer is ovarian cancer. In some embodiments, the cancer is melanoma. In some embodiments, the cancer is a solid tumor.

In another aspect, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises a telomerase antigen, thereby treating the cancer. In some embodiments, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises a human telomerase, thereby treating the cancer. In some embodiments, the cancer is acute myelogenous leukemia (AML).

In another aspect, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises ILAKFLHWL (SEQ ID NO: 658).

In another aspect, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises RLVDDFLLV (SEQ ID NO: 659).

In another aspect, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises RPGLLGASVLGLDDI (SEQ ID NO: 663).

In another aspect, the present disclosure provides a method of treating a subject having cancer, comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises LTDLQPYMRQFVAHL (SEQ ID NO: 664).

In some embodiments, the cancer is acute myelogenous leukemia (AML).

In another aspect, the present disclosure provides a method of treating a subject having acute myelogenous leukemia (AML), comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises PR1 (VLQELNVTV (SEQ ID NO: 225)). In another aspect, the present disclosure provides a method of treating a subject having acute myelogenous leukemia (AML), comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises PR1 (VLQELNVTV (SEQ ID NO: 225)), and wherein the erythroid cell further presents, e.g. comprises on the cell surface, at least one exogenous polypeptide comprising a costimulatory polypeptide. In another aspect, the present disclosure provides a method of treating a subject having acute myelogenous leukemia (AML), comprising administering to the subject an effective number of aAPCs comprising an erythroid cell (e.g. an enucleated erythroid cell), comprising one or more exogenous antigenic polypeptides, wherein one of the one or more exogenous antigenic polypeptides comprises PR1 (VLQELNVTV (SEQ ID NO: 225)), and wherein the erythroid cell further presents, e.g. comprises on the cell surface, at least one exogenous polypeptide comprising 4-1BBL. In another aspect, the present disclosure provides a method of treating a subject having acute myelogenous leukemia (AML), comprising administering to the subject an effective number of aAPCs comprising at least one exogenous antigenic polypeptide, PR1 (VLQELNVTV (SEQ ID NO: 225)), fused to an exogenous antigen presenting polypeptide, MHCI HLA-A2, fused to the GPA transmembrane domain (GPA), and wherein the erythroid cell further presents, e.g. comprises on the cell surface, at least one exogenous polypeptide comprising a costimulatory polypeptide. In another aspect, the present disclosure provides a method of treating a subject having acute myelogenous leukemia (AML), comprising administering to the subject an effective number of aAPCs comprising at least one exogenous antigenic polypeptide, PR1 (VLQELNVTV (SEQ ID NO: 225)), fused to an exogenous antigen presenting polypeptide, MHCI HLA-A2, fused to the GPA transmembrane domain (GPA), and wherein the erythroid cell further presents, e.g. comprises on the cell surface, at least one exogenous polypeptide comprising 4-1BBL.

The present disclosure is not limited to a certain type of cancer, but rather any cancer is contemplated as being treated by the aAPCs described herein. In certain embodiments, the cancer includes, but is not limited to, a cancer selected from acute lymphoblastic leukemia (ALL), ACUTE myeloid leukemia (AML), anal cancer, bile duct cancer, bladder cancer, bone cancer, bowel cancer, brain tumors, breast cancer, cancer of unknown primary, cancer spread to bone, cancer spread to brain, cancer spread to liver, cancer spread to lung, carcinoid, cervical cancer, choriocarcinoma, chronic lymphocytic leukemia (CLL), chronic myeloid leukemia (CML), colon cancer, colorectal cancer, endometrial cancer, eye cancer, gallbladder cancer, gastric cancer, gestational trophoblastic tumors (GTT), hairy cell leukaemia, head and neck cancer, hodgkin lymphoma, kidney cancer, laryngeal cancer, leukaemia, liver cancer, lung cancer, lymphoma, melanoma skin cancer, mesothelioma, men's cancer, molar pregnancy, mouth and oropharyngeal cancer, myeloma, nasal and sinus cancers, nasopharyngeal cancer, non hodgkin lymphoma (NHL), oesophageal cancer, ovarian cancer, pancreatic cancer, penile cancer, prostate cancer, rare cancers, rectal cancer, salivary gland cancer, secondary cancers, skin cancer (non melanoma), soft tissue sarcoma, stomach cancer, testicular cancer, thyroid cancer, unknown primary cancer, uterine cancer, vaginal cancer, and vulval cancer.

In certain embodiments, the cancer is a leukemia, e.g. AML or ALL. In other embodiments, the cancer is a hepatic cell carcinoma. In still other embodiments, the cancer is selected from a cervical cancer, head and neck cancer, lymphomas, and kidney clear cell carcinoma.

A neoantigen is a class of tumor antigens that arises from a tumor-specific mutation(s) which alters the amino acid sequence of genome encoded proteins. A neoantigen can include a polypeptide sequence or a nucleotide sequence. A mutation can include a frameshift or non-frameshift indel (insertion or deletion), missense or nonsense substitution, splice site alteration, genomic rearrangement or gene fusion, or any genomic or expression alteration giving rise to a neoORF. A mutation can also include a splice variant. Post-translational modifications specific to a tumor cell can include aberrant phosphorylation. Post-translational modifications specific to a tumor cell can also include a proteasome-generated spliced antigen. See Liepe et al., A large fraction of HLA class I ligands are proteasome-generated spliced peptides; Science. 2016 Oct. 21; 354 (6310):354-358. A tumor neoantigen is a neoantigen present in a subject's tumor cell or tissue but not in the subject's corresponding normal cell or tissue.

Recent analyses of The Cancer Genome Atlas (TCGA) datasets have linked the genomic landscape of tumors with tumor immunity, implicating neoantigen load in driving T cell responses (Brown et al., Genome Res. 2014 May; 24(5):743-50, 2014) and identifying somatic mutations associated with immune infiltrates (Rutledge et al., Clin Cancer Res. 2013 Sep. 15; 19(18):4951-60, 2013). Rooney et al. (2015 Jan. 15; 160(1-2):48-61) suggest that neoantigens and viruses are likely to drive cytolytic activity, and reveal known and novel mutations that enable tumors to resist immune attack.

In some embodiments, the antigen is a neoantigen identified from a cancer cell in a subject. In some embodiments, the neoantigen is a shared neoantigen. Methods of identifying neoantigens are known in the art and described, e.g., in U.S. Pat. No. 10,055,540, incorporated by reference in its entirety herein. Neoantigenic polypeptides and shared neoantigenic polypeptides are described, for example, in PCT/US2016/033452, U.S. Publication No. 20180055922, Schumacher and Hacohen et al. (Curr Opin Immunol. 2016 August; 41:98-103), Gubin, M M et al. (Nature. 2014 Nov. 27; 515(7528):577-81), Schumacher and Schreiber, Science. 2015 Apr. 3; 348(6230):69-74), Ott P A., et al., Nature. 2017 Jul. 13; 547(7662):217-221, all of which are incorporated by reference in their entireties herein.

Accordingly, in some embodiments, the exogenous antigenic polypeptide is a neoantigen polypeptide. In some embodiments, the exogenous antigenic polypeptide is a neoantigen polypeptide set forth in The Comprehensive Tumor-Specific Neoantigen Database (TSNAdb v1.0); available at biopharm.zju.edu.cn/tsnadb and described in Wu et al., Genomics Proteomics Bioinformatics 16 (2018) 276-282. In some embodiments, the exogenous antigenic polypeptide is a neoantigen polypeptide set forth in U.S. Pat. No. 10,055,540, incorporated by reference in its entirety herein. In some embodiments, the neoantigen polypeptide is selected from a polypeptide listed in Table 14.

TABLE 14
Neoantigen Polypeptides

| HLA class I | |
|---|---|
| allele | Neoantigen Peptide |
| HLA-A*01:01 | YEMFNDKSF (SEQ ID NO: 874) |
| HLA-A*02:01 | HRQEIFSHDFJ (SEQ ID NO: 875) |
| HLA-B*07:01 | FJIEJFOES (SEQ ID NO: 876) |
| HLA-B*08:01 | NEIOREIREI (SEQ ID NO: 877) |
| HLA-C*01:03 | JFKSIFEMMSJDSSU (SEQ ID NO: 878) |
| HLA-C*01:04 | KNFLENFIESOFI (SEQ ID NO: 879) |

In some embodiments, the neoantigen polypeptide is selected from a polypeptide listed in Table 15.

TABLE 15
Neoantigen Polypeptides

| Gene | HLA class I | |
|---|---|---|
| (Mutation) | allele | Neoantigen Peptide |
| BRAF | HLA-A*03:01 | KIGDFGLATEK (SEQ ID NO: 880) |
| (V600E) | | |
| KRAS (G12D) | HLA-A*02:01 | KLVVVGADGV (SEQ ID NO: 881) |
| KRAS (G12V) | HLA-A*02:01 | KLVVVGAVGV (SEQ ID NO: 882) |
| KRAS (G12V) | HLA-A*02:01 | KLVVVGAV (SEQ ID NO: 883) |
| BRAF | HLA-A*11:01 | KIGDFGLATEK (SEQ ID NO: 884) |
| (V600E) | | |
| PIK3CA | HLA-C*07:01 | ARHGGWTTKM (SEQ ID NO: 885) |
| (H1047R) | | |
| PIK3CA | HLA-C*07:02 | ARHGGWTTKM (SEQ ID NO: 886) |
| (H1047R) | | |
| PIK3CA | HLA-A*03:01 | STRDPLSEITK (SEQ ID NO: 887) |
| (E545K) | | |
| BRAF | HLA-B*57:01 | FGLATEKSRW (SEQ ID NO: 888) |
| (V600E) | | |
| BRAF | HLA-B*57:01 | LATEKSRW (SEQ ID NO: 889) |
| (V600E) | | |
| H3F3A | HLA-A*0201 | RMSAPSTGGV (SEQ ID NO: 890) |
| (K27M) | | |

For highly aggressive midline gliomas, a recurrent point mutation in the histone-3 gene (H3F3A) causes an amino acid change from lysine to methionine at position 27 (K27M). Ochs et al. (Oncoimmunology. 2017; 6(7):e1328340, incorporated by reference in its entirety herein) have shown that a peptide vaccine against K27M-mutant histone-3 is capable of inducing effective, mutation-specific, cytotoxic T-cell- and T-helper-1-cell-mediated immune responses in a major histocompatibility complex (MHC)-humanized mouse model. Accordingly, in some embodiments, the neoantigen polypeptide is H3F3A (K27M) (SEQ ID NO: 890).

In some embodiments, the cancer is a cancer associated with an oncogenic virus, for example Epstein Barr virus (EBV), hepatitis B and C (HBV and HCV), human papilloma virus (HPV), Kaposi sarcoma virus (KSV), and polyoma viruses. In other certain embodiments, the cancer is a cancer where retrovirus epitopes are identified. Cancers which are associated with a virus and which may be treated using the methods of the invention include, but are not limited to, cervical cancer, head and neck cancer, lymphomas, and kidney clear cell carcinoma.

Human leukocyte antigen (HLA) molecules are required for the immune recognition and subsequent killing of neoplastic cells by the immune system, as tumor antigens must be presented in an HLA-restricted manner to be recognized by T-cell receptors. Some tumor cells use aberrant expression of non-classical HLA-I molecules (HLA-E and HLA-G), which function as inhibitor ligands for immune-competent cells, to escape immune recognition and facilitate tumor immune escape (Moreau et al., Cell Mol Life Sci. 2002 September; 59(9):1460-6, incorporated by reference in its entirety herein). HLA class I histocompatibility antigen, alpha chain G (HLA-G) is a non-classical MHC class I molecule comprising a heavy a chain comprising one of more of alpha1, alpha2, and alpha3 domains.

In some embodiments, an artificial antigen presenting cell comprises on the cell surface an exogenous antigenic polypeptide, wherein the exogenous antigenic polypeptide is an HLA-G-derived polypeptide. In some embodiments, the HLA-G derived polypeptides are selected from the polypeptides shown below:

(SEQ ID NO: 891)
HLA-G146-154-DYLALNEDL
(SEQ ID NO: 892)
HLA-G194-202-RYLENGKEM
(SEQ ID NO: 893)
HLA-G139-148-RYAYDGKDYL
(SEQ ID NO: 894)
HLA-G141-150-AYDGKDYLAL

In some embodiments, the peptides shown above (SEQ ID NOs 891-894) bind to the a specific HLA-A allele: HLA-A*24:02 (e.g., HLA-A*24:02:01:01). In some embodiments, the peptides shown above (SEQ ID NOs 891-894) bind to other HLA alleles described herein.

In one particular embodiment, an artificial antigen presenting cell comprises an erythroid cell, wherein the erythroid cell presents, e.g. comprises on the cell surface, at least one exogenous antigenic polypeptide, PR1, a human leukocyte antigen (HLA)-A2 restricted peptide, fused to an exogenous antigen presenting polypeptide, MHCI HLA-A2, fused to the GPA transmembrane domain (GPA) (PR1-HLA-A2-GPA). In some embodiments, the erythroid cell is an enucleated cell. In some embodiments, the erythroid cell is a nucleated cell.

In certain embodiments, the aAPCs of the disclosure are used to treat highly vascularized tumors. Without being bound by theory, greater vascularization renders the tumors more accessible to the aAPCs comprising enucleated cells (e.g. erythroid cells) of the present disclosure. Tumor vascularity can be measured, for example, by intercapillary distance (thought to reflect tumor oxygenation) and microvessel density (provides a histological assessment of tumor angiogenesis). A highly vascular tumor can be any tumor of vascular origin, for example a hemangioma, a lymphangioma, a hemangioendothelioma, Kaposi sarcoma, an angiosarcoma, a hemangioblastoma.

In other embodiments, the aAPCs of the disclosure are used to treat tumors with leaky vasculature. There is general agreement that blood vessels in tumors are abnormal. One manifestation of this abnormality is a defective and leaky endothelium. Blood vessel leakiness not only influences the internal environment of tumors and perhaps the rate of angiogenesis, but it also governs access of therapeutics. Without being bound by theory, a more leaky blood vessel would provide more access to the aAPCs comprising enucleated cells (e.g. erythroid cells) of the present disclosure.

Autoimmune Diseases

Over the past two decades, considerable progress has been made in the treatment of a range of autoimmune disorders with many patients enjoying an improvement in quality of life as a result. Despite their success, current therapeutic approaches to autoimmune diseases are either generally or specifically immunosuppressive, and expose patients to an increased risk of opportunistic infection and hematological cancers, as is the case with JAK inhibitors, anti-TNF antibodies and anti-CD20 targeted antibodies. In up to one-third of cases, patients with autoimmune disorders fail to respond to treatment, and most responding patients ultimately lose response over time.

While the triggers of most autoimmune diseases remain unknown, it is generally understood that clinical disease is the result of a loss of tolerance to one's own cells. The accepted model of disease assumes a genetic susceptibility triggered by an environmental event, which leads to a breakdown of T-cell-mediated immune suppression. In principle, restoration of peripheral tolerance should provide patients with a complete or partial cure.

A range of competitive approaches to peripheral tolerance restoration have been investigated over the years. These include the oral administration and direct injection of a protein or peptide with or without immunosuppression, the creation of peptide bearing nanoparticles and the adoptive transfer of engineered regulatory T cells. Thus far, these approaches have not proven to be successful in late stage clinical trials, but the field continues to progress. Direct administration of peptides and nanoparticles suffer biodistribution, stability, presentation and orientation challenges which limit the effectiveness of cell-to-cell signaling. To date, adoptive transfer approaches are all autologous and are hampered by some of the same handling and scalability issues that limit the application of other cellular therapies.

Thus, in certain embodiments, the aAPCs of the present disclosure provide a novel and improved method of treating autoimmune diseases. The methods provided for treating autoimmune diseases have numerous advantages, including, for example, effective presentation of antigen on the surface of a cell (erythroid cell) together with an antigen-presenting polypeptide, e.g., MHC I or II and the extremely long half life of aAPCs in the circulation, thus providing extended exposure to the properly presented antigen.

Accordingly, in certain embodiments, the present disclosure provides an aAPC comprising an erythroid cell (e.g. an enucleated erythroid cell), comprising one or more exogenous polypeptides, wherein one of the one or more exogenous polypeptides comprises an autoimmune disease therapeutic. In some embodiments, the aAPC comprises a first exogenous polypeptide comprising a first autoimmune therapeutic and a second exogenous polypeptide, comprising a second autoimmune therapeutic. In some embodiments, the aAPC comprises a first exogenous polypeptide comprising a first autoimmune therapeutic, a second exogenous polypeptide, comprising a second autoimmune therapeutic, and a third exogenous polypeptide, comprising a third autoimmune therapeutic. In some embodiments, the erythroid cell is an enucleated cell. In some embodiments, the erythroid cell is a nucleated cell.

The first, second and third autoimmune therapeutic can act on the same target, for example a cell surface receptor and/or an endogenous human protein. Alternatively, the first, second and third anti-cancer therapeutic can act on different targets. The first, second or third targets may be members of the same biological pathway, wherein optionally the targets are cell surface receptors and/or endogenous human proteins. The first, second or third targets may be on different cell types. In embodiments, the first, second and/or third autoimmune therapeutic is a first, second and/or third exogenous polypeptide as described herein. For example, in some embodiments, the first exogenous polypeptide localizes the engineered erythroid cell to a desired site, e.g., a human cell, and the second exogenous polypeptide has a therapeutic activity, e.g., antigen presenting activity.

In some embodiments, the first exogenous polypeptide comprises an antigen-presenting polypeptide, e.g., an MHC molecule (e.g. MHCI or MHCII), and the second exogenous polypeptide comprises an antigen. In some embodiments, the MHCI is selected from HLA A, HLA B, and HLA C. In some embodiments, the MHCII is selected from HLA-DPα, HLA-DPβ, HLA-DM, HLA DOA, HLA DOB, HLA DQα, HLA DQβ, HLA DRα, and HLA DRβ. It will be recognized by one of skill in the art that, in preferred embodiments, the antigen is associated with or is the cause or trigger of the autoimmune disorder. For example, the antigen may be a self-antigen to which the autoimmune response is directed. In some embodiments, the antigen is selected from the antigens listed in Table 16, or an antigenic-portion thereof. In some embodiments, the antigen is selected from the antigens listed in Table 17, or an antigenic-portion thereof. In some embodiments, the antigen is selected from the antigens listed in Table 18, or an antigenic-portion thereof. In the provided methods for treating an autoimmune disorder, where the aAPC comprises an antigen-presenting polypeptide and an antigen listed in Tables 16, 17 or 18, or antigenic-portion thereof, the aAPC may be administered to a subject to treat the autoimmune disorder corresponding to the antigen as provided in Tables 16, 17 or 18.

In one aspect, the aAPCs are designed to suppress undesired T cell activity associated with or driving the autoimmune disorder. Thus, in some embodiments, the aAPC further comprises at least one exogenous co-inhibitory polypeptide as described herein. In some embodiments, the exogenous co-inhibitory polypeptide is selected from the co-inhibitory polypeptides listed in Table 7. In some embodiments, the exogenous co-inhibitory polypeptide is selected from IL-35, IL-10, VSIG-3 and a LAG3 agonist. In some embodiments, the co-inhibitory polypeptide suppresses an autoreactive T cell.

In another aspect, the aAPCs are designed to stimulate T regulatory cells, thereby biasing the immune system back to a more tolerogenic state. Thus, in some embodiments, the aAPC further comprises at least one costimulatory polypeptide as described herein. In this aspect, in preferred embodiments the at least one exogenous costimulatory polypeptide expands regulatory T-cells (Tregs) and is, e.g., an exogenous Treg costimulatory polypeptide as described herein. In some embodiments, the exogenous costimulatory polypeptide is selected from the costimulatory polypeptides listed in Table 10. In some embodiments, the costimulatory polypeptide is selected from the costimulatory polypeptides listed in Table 11.

In yet another aspect, the aAPCs are designed to expand and stimulate T cells, e.g., cytotoxic CD8+ T cells. In this aspect, the autoimmune disorder is preferably an autoimmune disorder caused or triggered by an infectious agent. In embodiments, the aAPC further comprises at least one exogenous costimulatory polypeptide as described herein. In embodiments, the at least one exogenous costimulatory polypeptide expands cytotoxic CD8+ T cells. In some embodiments, the exogenous costimulatory polypeptide is selected from the costimulatory polypeptides listed in Table 6. In some embodiments, the costimulatory polypeptides is selected from the group consisting of 4-1BBL, LIGHT, anti CD28, CD80, CD86, CD70, OX40L, IL-7, IL-12, GITRL, TIM4, SLAM, CD48, CD58, CD83, CD155, CD112, IL-15Rα fused to IL-15, IL-21, ICAM-1, a ligand for LFA-1, anti CD3, and a combination thereof. This aspect is described in greater detail below.

In some aspects, the disclosure provides a method of treating a subject having an autoimmune disease, comprising administering to the subject an effective number of the erythroid cells described herein to the subject, thereby treating the autoimmune disease. In various embodiments, the autoimmune disease may be an autoimmune disease provided in Tables 16, 17 or 18. In such methods, the aAPC useful for treating the autoimmune disorder comprises an exogenous antigen-presenting polypeptide and an antigenic polypeptide, wherein the antigenic polypeptide may be an antigen listed in Tables 16, 17 or 18, or antigenic-portion thereof, wherein the aAPC comprising the antigen as provided in Tables 16, 17 or 18 is used to treat the corresponding autoimmune disorder listed in Tables 16, 17 or 18.

For example, in some embodiments, the disclosure provides a method of treating a subject having the autoimmune disease Multiple Sclerosis (MS). In some embodiments, an artificial antigen presenting cell comprises an erythroid cell, wherein the erythroid cell presents, e.g. comprises on the cell surface, at least one exogenous antigenic polypeptide, wherein the antigenic peptide is MOG. In one particular embodiment, an artificial antigen presenting cell comprises an erythroid cell, wherein the erythroid cell presents, e.g. comprises on the cell surface, at least one exogenous antigenic polypeptide, wherein the antigenic peptide is MOG, and an exogenous antigen-presenting polypeptide, which is a MHCII single chain fusion, wherein the antigen-presenting polypeptide is fused to GPA (MOG-MHCII-GPA). In some embodiments, an artificial antigen presenting cell comprises an erythroid cell, wherein the erythroid cell presents, e.g. comprises on the cell surface, at least one exogenous antigenic polypeptide, wherein the antigenic peptide is MOG, an exogenous antigen-presenting polypeptide, wherein the exogenous antigenic polypeptide is specifically bound to the exogenous antigen-presenting polypeptide, which is a MHCII single chain fusion, and at least one Treg expansion/costimulatory polypeptide. In some embodiments, the Treg expansion/costimulatory peptide is CD25-specific IL-2. In a further embodiment, an artificial antigen presenting cell comprises an erythroid cell, wherein the erythroid cell presents, e.g. comprises on the cell surface, at least one exogenous antigenic polypeptide, wherein the antigenic peptide is MOG, an exogenous antigen-presenting polypeptide, wherein the exogenous antigenic polypeptide is specifically bound to the exogenous antigen-presenting polypeptide, which is a MHCII single chain fusion, and at least one coinhibitory polypeptide. In some embodiments, the coinhibitory polypeptide is PD-L1.

In some embodiments, the erythroid cell is an enucleated cell. In some embodiments, the erythroid cell is a nucleated cell.

In some embodiments, the autoimmune disease is a single antigen disease. Examples of single antigen diseases and their related antigens are shown in Table 16, below.

TABLE 16
Autoimmune diseases and associated single antigens

| Disease | Antigen |
|---|---|
| Neuromyelitis Optica (NMO) | Aquaporin 4 (AQP4) |
| Myasthenia Gravis (MG) | Acetylcholine receptor (AchR) |
| Membranous glomerulonephritis | Phospholipase A2 receptor (PLA2R) |
| Pemphigus Vulgaris (PV) | desmoglein 3 (DSG3) |
| Pemphigus Foliaceus (PF) | desmoglein 1 (DSG1) |

In another embodiments, the autoimmune disease is a multi-antigen disease. In embodiments wherein the autoimmune disease is a multi-antigen disease, the subject may be treated with aAPCs targeting more than one, e.g., two, three, four or more, of the antigens. It will be recognized by the skilled artisan that the multiple antigens may be present on the same aAPC, or they may be present on distinct aAPCs wherein a combination of the two, three, four or more distinct aAPCs comprising a single antigen are administered to the subject to treat the disorder. Examples of multi-antigen diseases and their related antigens are shown in Table 17, below.

TABLE 17
Autoimmune diseases and associated multi-antigens

| Disease | Antigen |
|---|---|
| Type I diabetes mellitus (T1DM) | Insulin/proinsulin/preproinsulin |
| Type I diabetes mellitus (T1DM) | glutamate decarboxylase (GAD65) |
| Type I diabetes mellitus (T1DM) | insulinoma antigen-2 (IA-2) |
| Multiple Sclerosis (MS) | myelin oligodendrocyte glycoprotein |
| | (MOG) |
| Multiple Sclerosis (MS) | myelin basic protein (MBP) |
| Multiple Sclerosis (MS) | proteolipid protein (PLP) |
| anti-phospholipid syndrome | beta-2 glycoprotein 1 (b2GP1) |
| (APS)/CAPS | |
| celiac disease | A-gliadin |

In certain embodiments, the autoimmune disease is selected from the group consisting of pemphigus vulgaris, myasthenia gravis, neuromyelitis optica, bullous pemphigoid, celiac disease multiple sclerosis, type 1 diabetes, rheumatoid arthritis, and membranous glomerulonephritis. In some embodiments, the autoimmune disease is selected from those listed in Table 18, below.

TABLE 18
Antigens for MS, Type 1 Diabetes, RA, and Membranous Nephritis

| Disease | Known antigen |
|---|---|
| Acute rheumatic fever | cross reactive antibodies to cardiac muscle |
| alopecia areata | Trychohyalin, keratin 16 |
| ANCA-associated vasculitis | Neutrophil cytoplasmic antigen, proteinase 3, |
| | myeloperodixase, bacterial permiability increasing factor |
| autoimmune gastritis | H,K adenosine triphosphatase |
| Autoimmune hemolytic | Rh blood group antigens, 1 antigen |
| anemia | |
| autoimmune hepatitis | nuclear protein, liver-kidney microsome type 1, liver cytosol |
| | type 1 |
| autoimmune myocarditis | cardiac myosin |
| Autoimmune thyroiditis | Thyroid peroxidase, thyroglobulin, thyroid-stimulating |
| | hormone receptor |
| Autoimmune uveitis | Retinal arrestin (S-antigen) |
| dermatomyositis | Mi2 ATPase |
| diabetes (type 1) | Pancreatic beta cell antigen |
| good pasture's syndrome | Noncollagenous domain of basement membrane collagen |
| | type IV |
| Graves' disease | Thyroid stimulating hormone receptor |
| Guillain-Barré syndrome | Neurofascin-18G, gliomedin, nodal adhesion molecueles |
| Hypoglycemia | Insulin receptor |
| Idiopathic thrombocytopenic | Platelet integrin Gpllb, Gpllla |
| purpura | |
| Insulin resistant diabetes | Insulin receptor |
| Membranous nephritis | Phospholipase AZ |
| mixed essential | rheumatoid factor IgG complexes |
| cryoglobulinemia | |
| multiple sclerosis | Myelin basic protein, proteolipid protein, myelin |
| | oligodendrocyte glycoprotein |
| myasthenia gravis | Acetylcholine receptor |
| Myasthenia gravis-MUSC | Muscarinic receptor |
| pemphigus/pemphigoid | Epidermal cadherin |
| pernicious anemia | intrinsic factor (Gastric) |
| polymyositis | nuclear and nucleolar antigen |
| primary biliary cirrhosis | neutrophil nuclear antigen, mitochondrial multienzyme |
| | complex |
| psoriasis | PSO p27 |
| rheumatoid arthritis | rheumatoid factor IgG complexes, synovial joint antigen, |
| | citrullinated protein, carbamylated protein |
| scleroderma/systemic | Sc1-86, nucleolar scleroderma antigen |
| sclerosis | |
| Sjogren's syndrome | SS-B, tupus La protein |
| systemic lupus erythematosus | DNAr histones, ribosomes, snRNP, scRNP |
| vitiligo | VIT-90, VIT-75, VIT-40 |
| Wegener's granulomatosis | neutrophil nuclear antigen |
| Antiphospholipid syndrome | Beta-2 glycoprotein 1 |
| (APS) & catastrophic APS | |
| Chemotherapy induced | Neuronal antigens |
| peripheral neuropathy | |
| Atypical hemolytic uremic | Complement factor H |
| syndrome | |
| Thrombotic | ADAMTS13 |
| thrombocytopenic purpura | |

In certain embodiments, an aAPC of the invention is used to drive tolerance induction in subjects with Type I diabetes. In one particular embodiment, an artificial antigen presenting cell comprises an erythroid cell, wherein the erythroid cell presents, e.g. comprises on the cell surface, at least one exogenous antigenic polypeptide, wherein the antigenic peptide is insulin B-chain, and in particular a portion of the insulin B-chain. In certain embodiments, the portion of the insulin B-chain is amino acids 9-23 of insulin B-chain. In some embodiments, the erythroid cell is an enucleated cell. In some embodiments, the erythroid cell is a nucleated cell.

Diseases of immune activation also include inflammatory diseases, such as, e.g. Crohn's disease, ulcerative colitis, celiac disease, or other idiopathic inflammatory bowel disease. Diseases of immune activation also include allergic diseases, such as, e.g. asthma, peanut allergy, shellfish allergy, pollen allergy, milk protein allergy, insect sting allergy, and latex allergy, animal dander allergy, black and English walnut allergy, brazil nut allergy, cashew nut allergy, chestnut allergy, dust mite allergy, egg allergy, fish allergy, hazelnut allergy, mold allergy, pollen allergy, grass allergy, shellfish allergy, soy allergy, tree nut allergy and wheat allergy.

Diseases of immune activation also include immune activation in response to a therapeutic protein administered to treat a primary condition, that lessens the efficacy of therapeutic protein, such as, e.g., clotting factor VIII in hemophilia A, clotting factor IX in hemophilia B, antitumor necrosis factor alpha (TNFα) antibodies in rheumatoid arthritis and other inflammatory diseases, glucocerebrosidase in Gaucher's disease, any recombinant protein used for enzyme replacement therapy, or asparaginase in acute lymphoblastic leukemia (ALL).

In some embodiments a patient is suffering from an autoimmune disease or condition or a self-antibody mediated disease or condition, in which the patient's immune system is active against an endogenous (self) molecule, for example a protein antigen, such that the immune system attacks the endogenous molecule, induces inflammation, damages tissue, and otherwise causes the symptoms of the autoimmune or self-antibody disease or conditions. The immune response might be driven by antibodies that bind to the endogenous molecule, or it may be driven by overactive T cells that attack cells expressing the endogenous molecule, or it may be driven by other immune cells such as regulatory T cells, NK cells, NKT cells, or B cells. In these embodiments, an antigenic protein or a fragment thereof corresponding to the endogenous (self) molecule may be expressed on an aAPC comprising an erythroid cell, presenting (e.g. comprising on the cell surface) one or more exogenous polypeptides, as described herein. The aAPCs, when administered once or more to the patient suffering from the disease or condition, would be sufficient to induce tolerance to the antigenic protein such that it no longer induced activation of the immune system, and thus would treat or ameliorate the symptoms of the underlying disease or condition. In certain embodiments, the aAPCs are used to stimulate T regulatory cells, thereby biasing the immune system back to a more tolerogenic state for the endogenous (self) molecule.

In some embodiments, a patient is suffering from an allergic disease, for example an allergy to animal dander, black walnut, brazil nut, cashew nut, chestnut, dust mites, egg, english walnut, fish, hazelnut, insect venom, latex, milk, mold, peanuts, pollen, grass, shellfish, soy, tree nuts, or wheat. A patient suffering from an allergy may mount an immune response upon contact with the antigenic fragment of the allergen, for example through diet, skin contact, injection, or environmental exposure. The immune response may involve IgE antibody, sensitized mast cells, degranulation, histamine release, and anaphylaxis, as well as canonical immune cells like T cells, B cells, dendritic cells, T regulatory cells, NK cells, neutrophils, and NKT cells. The allergic reaction may cause discomfort or it may be severe enough to cause death, and thus requires constant vigilance on the part of the sufferer as well as his or her family and caretakers. In these embodiments, the antigenic protein or a fragment thereof may be presented on an erythroid cell of the aAPC of the present disclosure. A population of these cells, when administered once or more to the patient suffering from the allergic disease or condition, would be sufficient to induce tolerance to the antigenic protein such that it no longer induced activation of the immune system upon exposure, and thus would treat or ameliorate the symptoms of the underlying allergic disease or condition.

Autoimmune Diseases Associated with an Infectious Agent

In some embodiments, the disclosure provides methods for treating an autoimmune disease or disorder associated with or triggered by an infectious agent. Exemplary autoimmune diseases or disorders associated with or triggered by infectious agents are provided in Table 19.

TABLE 19
Autoimmune diseases and associated infectious agents.

| Autoimmune Disease | Infectious Agent(s) |
|---|---|
| Allergic encephalitis | Measles virus |
| Autoimmune kidney disease | Streptococcal infections |
| Chagas disease | Trypanosoma cruzi |
| Chronic autoimmune hepatitis | Hepatitis C virus |
| Guillain-Barré syndrome | Campylobacter jejuni, Cytomegalovirus, |
| | Zika virus |
| Herpetic stromal keratitis | Herpes simplex virus |
| HTLV-associated myelopathy | Human T-cell leukemia virus |
| Lyme arthritis | Borrelia burgdorferi |
| Mixed cryoglobulinemia | Hepatitis C virus |
| Myocarditis | Coxsackie virus B3 |
| Pediatric autoimmune | Streptococcal infections |
| neuropsychiatric disorders | |
| Polyarteritis nodosa | Hepatitis B virus |
| Primary biliary cirrhosis | Escherichia coli |
| Reactive arthritis | Yersinia enterocolitica |
| Reiter's syndrome | Chlamydia trachomatis, Shigella species |
| Rheumatic fever | Streptococcus pyogenes |
| Rheumatic heart disease | Sreptococci |
| Rheumatoid arthritis | Normal gut flora |
| Scleroderma | Cytomegalovirus |
| Tourette syndrome | Streptococcal infections |
| Type 1 diabetes | Enterovirus, Rotavirus |
| Type 1 diabetes mellitus | Coxsackie virus B4 |

Any autoimmune disorder associated with an infectious agent, including without limitation those disorders presented in Table 19, is contemplated to be treated with an aAPC of the invention.

As provided by the present disclosure, an autoimmune disease associated with an infectious agent may be treated by administering to the subject an aAPC comprising a first exogenous polypeptide comprising an antigen-presenting polypeptide (e.g., an MHC molecule such as MHCI or MHCII), and a second exogenous polypeptide comprising an antigen or fragment thereof from the infectious agent (i.e., targeting the specific infectious agent triggering the disease).

In a particular embodiment, the aAPC useful for treating an autoimmune disorder associated with an infectious agent further comprises a third exogenous polypeptide comprising at least one costimulatory polypeptide. In an embodiment, the costimulatory polypeptide activates cytotoxic CD8+ T cells in order to target and eliminate cells infected with the infectious agent. For example, the cytotoxic CD8+ T cells may target, suppress and/or eliminate autoreactive B cells infected with the infectious agent. The costimulatory polypeptide may be any costimulatory polypeptide described herein. In embodiments, the at least one exogenous costimulatory polypeptide expands cytotoxic CD8+ T cells. In some embodiments, the exogenous costimulatory polypeptide is selected from the costimulatory polypeptides listed in Table 6. In some embodiments, the costimulatory polypeptides is selected from the group consisting of 4-1BBL, LIGHT, anti CD28, CD80, CD86, CD70, OX40L, IL-7, IL-12, GITRL, TIM4, SLAM, CD48, CD58, CD83, CD155, CD112, IL-15Rα fused to IL-15, IL-21, ICAM-1, a ligand for LFA-1, anti CD3, and a combination thereof.

In some embodiments, the autoimmune disease associated with an infectious agent is multiple sclerosis (MS). Several infectious agents have been associated with MS. Exemplary infectious agents associated with MS are provided in Table 20. In embodiments, the infectious agent associated with the autoimmune disorder is a virus.

TABLE 20
Infectious agents associated with
multiple sclerosis (MS)

| Infectious Agent | Antigenic Peptides |
|---|---|
| Borna virus | |
| Coronavirus | |
| Cytomegalovirus | |
| EBV | VLQWASLAV (SEQ ID NO: 698), |
| | FMVFLQTHI (SEQ ID NO: 699), |
| | FLQTHIFAEV (SEQ ID NO: 700), |
| | or SIVCYFMVFL |
| | (SEQ ID NO: 701) |
| Hepatitis B virus | |
| Herpes simplex virus, | |
| type 1 | |
| Herpes simplex virus, | |
| type 2 | |
| HHV-6 | PRTPPPS (SEQ ID NO: 719) |
| HTLV-1 | |
| Influenza A virus | |
| LM7 virus | |
| Measles virus | |
| MS1533 | |
| MSRV (HERV-W) | |
| Parainfluenza virus 1 | |
| Rabies virus | |
| Rubella virus | |
| Scrapie agent | |
| Simian virus 5 | |
| SMON-like virus | |
| Tick borne | |
| encephalitis virus | |
| Torque Teno virus | |
| VZV | |

In some embodiments, the present disclosure provides a method of treating a subject having MS, comprising administering to the subject an effective number of aAPCs comprising an erythroid cell (e.g. an enucleated erythroid cell), comprising one or more exogenous polypeptides, wherein one of the one or more exogenous polypeptides comprises an antigen from an infectious agent listed in Table 20, or an immunogenic peptide thereof, thereby treating the MS. In embodiments, the aAPCs further comprise an MHC molecule, and optionally a costimulatory polypeptide.

In a particular embodiment, the infectious agent associated with MS is Epstein-Barr virus (EBV). While not wishing to be bound by any particular theory, it is believed that during primary infection, EBV infects autoreactive naive B cells in tonsils driving them to enter germinal centers where they proliferate intensely and differentiate into latently infected autoreactive memory B cells, which then exit from the tonsils and circulate in the blood. The number of EBV-infected B cells is normally controlled by EBV-specific cytotoxic CD8+ T cells, which kill proliferating and lytically infected B cells, but not if there is a defect in this defense mechanism. Surviving EBV-infected autoreactive memory B cells enter the CNS where they take up residence and produce oligoclonal IgG and pathogenic autoantibodies, which attack myelin and other components of the CNS. Autoreactive T cells that have been activated in peripheral lymphoid organs by common systemic infections circulate in the blood and enter the CNS where they are reactivated by EBV-infected autoreactive B cells presenting CNS peptides (Cp) bound to major histocompatibility complex (MHC) molecules. These EBV-infected B cells provide costimulatory survival signals (B7) to the CD28 receptor on the autoreactive T cells and thereby inhibit activation-induced T-cell apoptosis, which normally occurs when autoreactive T cells enter the CNS and interact with nonprofessional antigen-presenting cells such as astrocytes and microglia which do not express B7 costimulatory molecules. After the autoreactive T cells have been reactivated by EBV-infected autoreactive B cells, they produce cytokines such as interleukin-2 (IL-2) interferon-γ (IFNγ) and tumor necrosis factor (TNF) and orchestrate an autoimmune attack on the CNS with resultant oligodendrocyte and myelin destruction.

Accordingly, in some embodiments, the present disclosure provides a method of treating a subject having MS, comprising administering to the subject an effective number of aAPCs comprising an erythroid cell (e.g. an enucleated erythroid cell), comprising one or more exogenous polypeptides, wherein one of the one or more exogenous polypeptides comprises an EBV antigen. In some embodiments, the present disclosure provides a method of treating a subject having MS, comprising administering to the subject an effective number of aAPCs comprising an erythroid cell (e.g. an enucleated erythroid cell), comprising one or more exogenous polypeptides, wherein one of the one or more exogenous polypeptides comprises an EBV antigen from Table 1. In some embodiments, the present disclosure provides a method of treating a subject having MS, comprising administering to the subject an effective number of aAPCs comprising an erythroid cell (e.g. an enucleated erythroid cell), comprising one or more exogenous polypeptides, wherein one of the one or more exogenous polypeptides comprises an EBV antigen selected from a gp350 or EBNA1 antigen. In some embodiments, the present disclosure provides a method of treating a subject having MS, comprising administering to the subject an effective number of aAPCs comprising an erythroid cell (e.g. an enucleated erythroid cell), comprising one or more exogenous polypeptides, wherein one of the one or more exogenous polypeptides comprises an EBV antigen selected from: VLQWASLAV (SEQ ID NO: 698), FMVFLQTHI (SEQ ID NO: 699), FLQTHIFAEV (SEQ ID NO: 700), SIVCYFMVFL (SEQ ID NO: 701), CLGGLLTMV (SEQ ID NO: 691), GLCTLVAML (SEQ ID NO: 692), FLYALALLL (SEQ ID NO: 693), YVLDHLIVV (SEQ ID NO: 694), RLRAEAQVK (SEQ ID NO: 695), AVFDRKSDAK (SEQ ID NO: 696), RPPIFIRLL (SEQ ID NO: 697). Such aAPCs are expected to eliminate the EBV-infected autoreactive B cells, to thereby treat the MS. In embodiments, the aAPCs further comprise an MHC molecule, and optionally a costimulatory polypeptide. In some embodiments, the antigen from EBV is gp350 or an immunogenic peptide thereof (e.g. HLA A2 peptide VLQWASLAV (SEQ ID NO: 698)). In some embodiments, the antigen from EBV is EBNA1 or an immunogenic peptide thereof (e.g. FMVFLQTHI (SEQ ID NO: 699), FLQTHIFAEV (SEQ ID NO: 700), or SIVCYFMVFL (SEQ ID NO: 701)). In some embodiments, the antigen from EBV is FMVFLQTHI (SEQ ID NO: 699). In some embodiments, the antigen from EBV is FLQTHIFAEV (SEQ ID NO: 700). In some embodiments, the antigen from EBV is SIVCYFMVFL (SEQ ID NO: 701).

In some embodiments, the present disclosure provides a method of treating a subject having MS, comprising administering to the subject an effective number of aAPCs comprising an erythroid cell (e.g. an enucleated erythroid cell), comprising one or more exogenous polypeptides, wherein one of the one or more exogenous polypeptides comprises gp350 or an immunogenic peptide thereof, thereby treating the MS. In some embodiments, the immunogenic peptide of gp350 comprises or consists of the HLA A2 peptide VLQWASLAV (SEQ ID NO: 698). In some embodiments, the immunogenic peptide of gp350 is capable of inducing cytotoxic CD8+ T cells that recognize the gp350 antigen.

In some embodiments, the present disclosure provides a method of treating a subject having MS, comprising administering to the subject an effective number of aAPCs comprising an erythroid cell (e.g. an enucleated erythroid cell), comprising one or more exogenous polypeptides, wherein one of the one or more exogenous polypeptides comprises EBNA1 or an immunogenic peptide thereof, thereby treating the MS. In some embodiments the immunogenic peptide of EBNA1 comprises or consists of a peptide selected from FMVFLQTHI (SEQ ID NO: 699), FLQTHIFAEV (SEQ ID NO: 700), and SIVCYFMVFL (SEQ ID NO: 701). In some embodiments, the immunogenic peptide of EBNA1 is capable of inducing cytotoxic CD8+ T cells that recognize the EBNA1 antigen.

As provided herein, there are several alternative ways in which an aAPC may be designed and used to treat an autoimmune disease associated with or triggered by an infectious agent in a subject. For example, an autoimmune disease associated with an infectious agent may alternatively be treated by administering to the subject an aAPC comprising a first exogenous polypeptide comprising an antigen-presenting polypeptide (e.g., an MHC molecule such as MHCI or MHCII), a second exogenous polypeptide comprising an antigen or fragment thereof, and optionally a third exogenous polypeptide comprising at least one co-inhibitory polypeptide, or at least one Treg costimulatory polypeptide (also referred to herein as Treg expansion polypeptide). In some embodiments, the antigen or antigenic fragment thereof is from the infectious agent (i.e., targeting the infectious agent triggering the disease). In some embodiments, the antigen or antigenic fragment thereof is an antigen associated with the infection-induced autoimmune disorder (e.g., a self-polypeptide).

In some embodiments, as described above more generally for any autoimmune disorder, the aAPC useful for treating an autoimmune disorder associated with an infectious agent comprises a Treg co stimulatory/expansion polypeptide. The Treg co stimulatory/expansion polypeptide may be any Treg expansion polypeptide described herein. In an embodiment, the Treg costimulatory/expansion polypeptide expands Tregs that, in turn, suppress T cells generated in the subject in response to the infectious agent.

In some embodiments, as described above more generally for any autoimmune disorder, the aAPC useful for treating an autoimmune disorder associated with an infectious agent comprises a co-inhibitory polypeptide. The co-inhibitory polypeptide may be any co-inhibitory polypeptide described herein. In an embodiment, the co-inhibitory polypeptide suppresses T cells generated in the subject in response to the infectious agent.

In another embodiment, the autoimmune disease is not associated with an infectious agent. For an autoimmune disease that is not associated with an infectious agent, one of skill in the art will recognize, based on the disclosure elsewhere herein, that the autoimmune disease may be treated by administering to the subject an aAPC comprising a first exogenous polypeptide comprising an antigen-presenting polypeptide (e.g., an MHC molecule such as MHCI or MHCII), a second exogenous polypeptide comprising an antigen or fragment thereof (e.g., an antigenic polypeptide from the endogenous (self) polypeptide to which the autoimmune activity is directed) and a third exogenous polypeptide comprising at least one coinhibitory polypeptide or at least one Treg expansion polypeptide. In these embodiments, the aAPCs are administered to the subject in order to induce peripheral tolerance to the antigen triggering the autoimmune response.

Infectious Diseases

In certain embodiments, the present disclosure provides an aAPC comprising an erythroid cell (e.g. an enucleated erythroid cell) or an enucleated cell, comprising one or more exogenous polypeptides, wherein one of the one or more exogenous polypeptides comprises an infectious disease therapeutic. In some embodiments, the erythroid cell is an enucleated erythroid cell. In some embodiments, the erythroid cell is a nucleated erythroid cell.

“Infectious disease therapeutic” as used herein, refers to an exogenous polypeptide which inhibits an infectious disease, e.g., reduces or alleviates a cause or symptom of an infectious disease, or improves a value for a parameter associated with the infectious disease, e.g., viral load or bacterial load. In embodiments, the infectious disease therapeutic is a first or second exogenous polypeptide, which when present or expressed with the other exogenous polypeptide, inhibits an infectious disease. In an embodiment, a first or second infectious disease therapeutic has activity in the absence of the other. In embodiments, the infectious disease therapeutic inhibits the infectious disease directly, e.g., by killing pathogens. In embodiments, the infectious disease therapeutic inhibits the infectious disease by stimulating a subject's immune response, e.g., as a vaccine.

In some embodiments, the aAPC comprises a first exogenous polypeptide comprising a first infectious disease therapeutic and a second exogenous polypeptide, comprising a second infectious disease therapeutic. In some embodiments, the aAPC comprises a first exogenous polypeptide comprising a first infectious disease therapeutic, a second exogenous polypeptide, comprising a second infectious disease therapeutic, and a third exogenous polypeptide, comprising a third infectious disease therapeutic.

The first, second and third infectious disease therapeutic can act on the same target, for example a cell surface receptor and/or an endogenous human protein. Alternatively, the first, second and third anti-cancer therapeutic can act on different targets. The first, second or third targets may be members of the same biological pathway, wherein optionally the targets are cell surface receptors, endogenous human proteins. The first, second or third targets may be on different cell types. In some embodiments, the first exogenous polypeptide localizes the engineered erythroid cell to a desired site, e.g., a human cell, and the second exogenous polypeptide has a therapeutic activity, e.g., antigen presenting activity.

In certain preferred embodiments, the infectious disease therapeutic is an antigen, e.g., an antigen from a pathogen or infectious agent (where “pathogen” and “infectious agent” are used interchangeably herein). In some embodiments, the first therapeutic is an antigen, e.g., an antigen from a pathogen. In some embodiments, the first therapeutic and second therapeutic is an antigen, e.g., an antigen from a pathogen. In certain embodiments, the first, second and third therapeutic is an antigen, e.g., an antigen from a pathogen.

In embodiments, the aAPC further comprises an additional exogenous polypeptide wherein the additional exogenous polypeptide comprises an antigen-presenting polypeptide, e.g., an MHC molecule (e.g. MHCI or MHCII). In embodiments, the aAPC further comprises an additional exogenous polypeptide, wherein the additional exogenous polypeptide comprises at least one costimulatory polypeptide. In some embodiments, the aAPC further comprises an additional exogenous polypeptide comprising an antigen-presenting polypeptide and an additional exogenous polypeptide comprising at least one costimulatory polypeptide.

In some embodiments, the aAPC comprises a first exogenous polypeptide comprises an antigen-presenting polypeptide, e.g., an MHC molecule (e.g. MHCI or MHCII), and the second exogenous polypeptide comprises an antigen. In various embodiments, the antigen is an antigen of a pathogen, e.g., a viral pathogen, a bacterial pathogen, a fungal pathogen, or a parasitic pathogen.

It is encompassed by the present invention that the antigen may be any pathogenic antigen, or antigenic-portion thereof, known in the art. Exemplary pathogens for which one or more antigenic peptides can be presented on an erythroid cell of the invention are described in detail below, but are not intended to be limiting. In various embodiments, the antigenic peptide is from a pathogen provided in any one of Tables 21, 22, 23 and 24 below. In some embodiments, the antigenic peptide is from a pathogen provided in Table 21. In some embodiments, the antigenic peptide is from a pathogen provided in Table 22. In some embodiments, the antigenic peptide is from a pathogen provided in Table 23. In some embodiments, the antigenic peptide is from a pathogen provided in Table 24. It will be recognized by the skilled artisan that an aAPC as provided herein, comprising an antigen from a particular pathogen or infectious agent, can be administered to a subject to treat an infection in the subject caused by that pathogen or infectious agent. It will be further recognized by the skilled artisan that an aAPC as provided herein, comprising an antigen from a particular pathogen or infectious agent, can be administered to a subject to treat a disease or disorder in the subject, wherein the disease, disorder is caused, directly or indirectly, by infection with that pathogen or infectious agent.

In some aspects, the disclosure provides an method of treating a subject having an infectious disease, comprising administering to the subject an effective number of the erythroid cells described herein to the subject, thereby treating the infectious disease.

In another aspect, the present disclosure provides a method of treating a subject having an infectious disease, comprising administering to the subject an effective number of aAPCs comprising an erythroid cell (e.g., an enucleated erythroid cell) or an enucleated cell, comprising one or more exogenous polypeptides, wherein one of the one or more exogenous polypeptides comprises a pathogenic antigen, thereby treating the infectious disease.

In certain embodiments, the infectious disease therapeutic is selected from an antimicrobial polypeptide listed on a publicly available bioinformatic database, such as CAMP, CAMP release 2 (Collection of sequences and structures of antimicrobial peptides), the Antimicrobial Peptide Database (found at aps.unmc.edu/AP/main.php), LAMP, BioPD, and ADAM (A Database of Anti-Microbial peptides) (found at bioinformatics.cs.ntou.edu.tw/adam/). The Antimicrobial peptide databases may be divided into two categories on the basis of the source of peptides it contains, as specific databases and general databases. These databases have various tools for antimicrobial peptides analysis and prediction. For example, CAMP contains AMP prediction, feature calculator, BLAST search, clustalW, VAST, PRATT, Helical wheel etc. In addition, ADAM allows users to search or browse through AMP sequence-structure relationships.

In certain embodiments, the infectious disease therapeutic is selected from a viral polypeptide. In these embodiments, an aAPC comprises an erythroid cell (e.g., an enucleated erythroid cell) or an enucleated cell, comprising one or more exogenous polypeptides, wherein one of the one or more exogenous polypeptides comprises an antigenic viral polypeptide, and the aAPC is administered to a subject to treat an infection with the virus.

Viral infections include adenovirus, coxsackievirus, hepatitis A virus, poliovirus, Epstein-Barr virus, herpes simplex type 1, herpes simplex type 2, human cytomegalovirus, human herpesvirus type 8, varicella-zoster virus, hepatitis B virus, hepatitis C viruses, human immunodeficiency virus (HIV), influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). Specific examples of these viruses include human respiratory coronavirus, influenza viruses A-C, hepatitis viruses A to G, and herpes simplex viruses 1-9.

Exemplary viral pathogens are shown below in Table 21. In certain embodiments, the viral pathogen is selected from Hepatitis B virus, Hepatitis C virus, Epstein Barr virus, Cytomegalovirus (CMV).

TABLE 21
Viral Pathogens

| | Examples, by | |
|---|---|---|
| Baltimore | Family | |
| classification | (Human Host) | Species/Pathology example |
| dsDNA viruses | Adenoviridae | Example Respiratory infection |
| dsDNA viruses | Polyomaviridae | Example - progressive multifocal |
| | | leukoencephalopathy |
| dsDNA viruses | Papiliomaviradae | Example - betapapilloma virus (warts, malignant |
| | | tumors) |
| dsDNA viruses | Poxviridae | Example - Molluscum contagiosum (skin lesions) |
| dsDNA viruses | Herpesvirales | Example - Varicellovirus (Chickenpox) |
| ssDNA viruses | Anelloviridae | Asymptomatic, may be associate with hepatitis, |
| (+strand or “sense”) | | pulmonary disease, hematological disorders, |
| DNA | | myopathy, lupus |
| ssDNA viruses | Parvoviridae | Example - Fifth disease |
| (+strand or “sense”) | | |
| DNA | | |
| dsRNA | Reoviridae | Example - Colorado tick fever |
| viruses (e.g. Reoviruses) | | |
| (+)ssRNA viruses | Coronaviridae | Example - Pneumonia, gastroenteritis |
| (+strand or sense) RNA | | |
| (+)ssRNA viruses | Picornaviridae | Example - Myocarditis |
| (+strand or sense) RNA | | |
| (+)ssRNA viruses | Astoviridae | Example - infantile gastroenteritis |
| (+strand or sense) RNA | | |
| (+)ssRNA viruses | Caliciviridae | Example - Norovirus - gastroenteritis |
| (+strand or sense) RNA | | |
| (+)ssRNA viruses | Flaviviridae | Example - Dengue, Zika |
| (+strand or sense) RNA | | |
| (+)ssRNA viruses | Hepeviridae | Example - Hepatits |
| (+strand or sense) RNA | | |
| (+)ssRNA viruses | Togaviridae | Example - Rubella |
| (+strand or sense) RNA | | |
| (−)ssRNA viruses | Rhabdoviridae | Example - Rabies |
| (−strand or antisense) | | |
| RNA | | |
| (−)ssRNA viruses | Filoviridae | Example - Ebola, Marburg |
| (−strand or antisense) | | |
| RNA | | |
| (−)ssRNA viruses | Paramyxoviridae | Example - Mumps |
| (−strand or antisense) | | |
| RNA | | |
| (−)ssRNA viruses | Pneumovirinae | Example - Respiratory tract infection |
| (−strand or antisense) | | |
| RNA | | |
| (−)ssRNA viruses | Arenaviridae | Example - Enchephalitis, Hemorrhagic fever |
| (−strand or antisense) | | |
| RNA | | |
| (−)ssRNA viruses | Bunyaviridae | Example - Hantavirus pulmonary syndrome/ |
| (−strand or antisense) | | hemorrhagic fever |
| RNA | | |
| (−)ssRNA viruses | Deltavirus | Example - hepatitis, cirrhosis |
| (−strand or antisense) | | |
| RNA | | |
| (−)ssRNA viruses | Orthomyxoviridae | Example - Influenza A, Influenza B |
| (−strand or antisense) | | |
| RNA | | |
| ssRNA-RT viruses | Retroviridae | Example - lentivirus - HIV |
| (+strand or sense) RNA | | |
| with DNA | | |
| intermediate in life- | | |
| cycle | | |
| dsDNA-RT viruses | Hepadnaviridae | Example disease - hepatitis, cirrhosis, hepatocellular |
| | | carcinoma |

In certain embodiments, the antigens are selected from viral, retroviral, and testes antigens. For example, in certain embodiments, the virus is selected from Epstein Barr virus (EBV), hepatitis B (HBV), hepatitis C (HCV), human papilloma virus (HPV), Kaposi sarcoma virus (KSV), and polyoma viruses. In some embodiments, the virus is hepatitis B (HBV). In some embodiments, the present disclosure provides a method of treating a subject having a cancer associated with an oncogenic virus (e.g. HPV), comprising administering to the subject an effective number of aAPCs comprising an erythroid cell (e.g. an enucleated erythroid cell), comprising one or more exogenous polypeptides, wherein one of the one or more exogenous polypeptides comprises an HPV antigen from Table 1. In some embodiments, the present disclosure provides a method of treating a subject having a cancer associated with an oncogenic virus (e.g. HPV), comprising administering to the subject an effective number of aAPCs comprising an erythroid cell (e.g. an enucleated erythroid cell), comprising one or more exogenous polypeptides, wherein one of the one or more exogenous polypeptides comprises an HPV-E7 antigen. In some embodiments, the present disclosure provides a method of treating a subject having a cancer associated with an oncogenic virus (e.g. HPV), comprising administering to the subject an effective number of aAPCs comprising an erythroid cell (e.g. an enucleated erythroid cell), comprising an exogenous polypeptide, wherein the exogenous polypeptide comprises YMLDLQPET (SEQ ID NO: 713), YMLDLQPETT (SEQ ID NO: 714), or TIHDIILECV (SEQ ID NO: 712). In some embodiments, the exogenous polypeptide comprises YMLDLQPET (SEQ ID NO: 713). In some embodiments, the exogenous polypeptide is YMLDLQPET (SEQ ID NO: 713).

In some embodiments, the present disclosure provides a method of treating a subject having a viral infection, comprising administering to the subject an effective number of aAPCs comprising an erythroid cell (e.g. an enucleated erythroid cell), comprising one or more exogenous polypeptides, wherein one of the one or more exogenous polypeptides comprises an antigen from an infectious agent listed in Table 21, or an immunogenic peptide thereof, thereby treating the viral infection. In embodiments, the aAPCs further comprise an MHC molecule, and optionally a costimulatory polypeptide.

In some embodiments, the present disclosure provides methods for treating hepatitis B infection, e.g., chronic HBV infection. It is estimated that up to 250 million people worldwide have chronic hepatitis B, which is primarily transmitted in utero or during childbirth. Chronic HBV causes cirrhosis and hepatocellular carcinoma. The pathology of HBV may be due to hepatitis B antigen-specific T cell recruitment of nonantigen-specific T cells, which secrete cytokines causing liver damage. It has been proposed that exhausted T cells are key to the etiology of chronic hepatitis. HBV-specific T cells have been reported to be reactivated by PD1 blockade, e.g., by anti-PD1, and/or IL-12 (Schirdewahn et al. J. Infect. Dis. 2017; Fisicaro et al Gastroenterology 2010; and Schurich PLOS Pathogens, 2013, the entire contents of each of which is incorporated herein by reference). Thus, it is envisioned by the present invention that aAPCs provided herein may be used to treat chronic HBV by reactivating HBV-specific T cells, e.g., for cytolytic or non-cytolytic anti-viral activity.

Accordingly, In some embodiments, the present disclosure provides a method of treating a subject having a Hepatitis B viral (HBV) infection, e.g., chronic hepatitis B, comprising administering to the subject an effective number of aAPCs comprising an erythroid cell (e.g. an enucleated erythroid cell), comprising one or more exogenous polypeptides, wherein one of the one or more exogenous polypeptides comprises an HBV-specific antigen, or an immunogenic peptide thereof, thereby treating the HBV infection. In some embodiments, the HBV infection is a chronic HBV infection. In embodiments, the aAPCs further comprise an exogenous antigen-presenting polypeptide, i.e., an MHC molecule, e.g., MHC class I or MHC class I single chain fusion. In some embodiments, the aAPC further comprises at least one exogenous costimulatory polypeptide. In embodiments, the at least one costimulatory polypeptide is selected from the group consisting of 4-1BBL, IL-2, IL-12, IL-15, IL-18, IL-21, and any combination thereof, e.g., IL-12 and IL-15, or 4-1BBL and IL-15. In some embodiments, the aAPC further comprises an additional exogenous polypeptide, wherein the additional exogenous polypeptide comprises a checkpoint inhibitor. In some embodiments, the checkpoint inhibitor is an antibody molecule to PD1. In a particular embodiment, the aAPC comprises an erythroid cell comprising one or more exogenous polypeptides, wherein the one or more exogenous polypeptides comprise: an exogenous antigenic polypeptide comprising an HBV-specific antigen, or an immunogenic peptide thereof, an exogenous antigen-presenting polypeptide, e.g., MHC class I or MHC class I single chain fusion, an exogenous costimulatory polypeptide, e.g., IL-12 or 4-1BBL, and a checkpoint inhibitor, e.g., antibody to PD1.

In certain embodiments, the infectious disease therapeutic is selected from a bacterial polypeptide. In these embodiments, an aAPC comprises an erythroid cell (e.g., an enucleated erythroid cell) or an enucleated cell, comprising one or more exogenous polypeptides, wherein one of the one or more exogenous polypeptides comprises an antigenic bacterial polypeptide, and the aAPC is administered to a subject to treat an infection with the bacteria.

Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella burnetii, chlamydia, Mycobacterium leprae, Salmonella; shigella; Yersinia enterocolitica; Yersinia pseudotuberculosis; Legionella pneumophila; Mycobacterium tuberculosis; Listeria monocytogenes; Mycoplasma spp.; Pseudomonas fluorescens; Vibrio cholerae; Haemophilus influenzae; Bacillus anthracis; Treponema pallidum; Leptospira; Borrelia; Corynebacterium diphtheriae; Francisella; Brucella melitensis; Campylobacter jejuni; Enterobacter; Proteus mirabilis; Proteus; and Klebsiella pneumoniae.

Exemplary bacterial pathogens are shown below in Table 22.

TABLE 22
Bacterial Pathogens

| Phylum | By Genus | Species/Pathology Example |
|---|---|---|
| Proteobacteria | Erlichia | Erlichosis (Pain, malaise, fever gastrointestinal disorder, |
| | | confusion rash. Can be fatal) |
| Proteobacteria | Rickettsia | Two groups of diseases from different subspecies - Spotted |
| | | fever; Typhus |
| Proteobacteria | Brucella | Brucellosis. 4 known species cause disease in humans. |
| | | Can be sporadic or chronic. Fever, malaise, lesions |
| Proteobacteria | Bartonella | Multiple species. Pathophysiology includes endocarditis, |
| | | neuroretinitis, cat scratch disease, peliosis hepititus, |
| | | general bactermia angiomatosis, Carrion's disease, Trench |
| | | fever |
| Proteobacteria | Bordetella | Multiple species. Adherence to ciliated ephilium drives |
| | | respiratory tract infections. B. pertussis = subspecies |
| | | example |
| Proteobacteria | Neisseria | Includes N meningitidis (bacterial meningitis and |
| | | septicemia) and N gonorrheae (Gonorrhea) - colonize |
| | | mucosal surfaces |
| Proteobacteria | Francisella | Examples include f. tularensis (tularemia), F novicida, F |
| | | philomiragia (septicemia) |
| Proteobacteria | Legionella | Examples include L pneumophila - legionnaires disease |
| | | and pontiac fever |
| Proteobacteria | Coxiella | Coxiella burnetii - Q Fever |
| Proteobacteria | Moraxella | Examples include M. catarrhalis (lower respiratory tract |
| | | infection) and Moraxella lacunata (blepharoconjunctivitis) |
| Proteobacteria | Pseudomonas | Multiple groups and subspecies therein. Examples include |
| | | P. aeruginosa - opportunistic infection in cystic fobrosis, |
| | | burn and immunocompromised patients. Colonizes skin, |
| | | lungs, kidney, urinary tract. Found on most medical |
| | | equipment - develops enduring biofilms |
| Proteobacteria | Vibrio | Multiple species - foodborne disease generally implicated |
| | | with gastroenteritis - example - V. cholerae. Can also |
| | | cause septicemia in open wounds |
| Proteobacteria | Plesimonas | Example P. Shigelloides - causes gastrointestinal disease |
| Proteobacteria | Aeromonas | Cornucopia of human disease including and not limited to |
| | | gastrointestinal disease, wound and soft tissue infection, |
| | | blood borne dyscrasias |
| Proteobacteria | Citrobacter | Examples: C. freundii, C. koseri, C. amalonaticus. Infect |
| | | urinary tract, can cause infant meningitis and sepsis |
| Proteobacteria | Enterobacter | Multiple species - Urinary and respiratory tract infections |
| | | are most common. Example E. aerogenes are a common |
| | | source of opportunistic and/or nosocomial infections |
| Proteobacteria | Escerichia | Common cause of gastrointestinal infections. Example E. coli |
| Proteobacteria | Klebsiella | Multiple species and sub-species. Cause a variety of |
| | | opporuntistic infections: Pneumonia, UTI, septicemia, |
| | | meningitis, diarrhea, soft tissue infection |
| Proteobacteria | Proteus | Urinary tract infections (including kidney) - P. vulgaris, P. mirabilis, |
| | | P. penneri |
| Proteobacteria | Providencia | Multiple species cause. Urinary tract infections. Example: |
| | | P. stuartii |
| Proteobacteria | Morganella | Single species with 2 subspecies. M. morganii. Cause |
| | | opportunistic infections (wound, UTI) |
| Proteobacteria | Salmonella | Two species S. bongori and S. enterica, along with |
| | | multiple subspecies. Typical non-typhoidal salmonella |
| | | causes gastrointestinal disease. In developing countries it |
| | | also causes blood infections. Typhiidal disease causes |
| | | typhoid fever, hypovolemic shock, septic shock |
| Proteobacteria | Serratia | Multiple species: Opportunisitc infection that often |
| | | develops biofilms. Colonizes respiratory and urinary tract. |
| | | Responsible for 2% of nosocomial infections of blood, |
| | | LRT, UT, surgical wounds, skin and soft tissue infection. |
| | | Example s. marcescens |
| Proteobacteria | Shigella | 4 species. Causes shigellosis (leading bacterial cause of |
| | | diarrhea). Example: S. dysenteriae |
| Proteobacteria | Yersinia | Multiple species and sub-species. Example: Y. pestis |
| | | causes the plague |
| Proteobacteria | Pasteurella | Multiple species. P. multocida species is the most frequent |
| | | example of human infection (-symptoms include |
| | | swelling, cellulitis, wound drainage and arthritis) |
| Proteobacteria | Hemophilus | Multiple species. Example H influenzae (Hib) causes |
| | | penumonia, sepsis and bacterial meningitis in young |
| | | children |
| Proteobacteria | Campylobacter | Multiple species. Cause campylobacteriosis |
| | | (gastrointestinal disease - inflammatory diarrhea/ |
| | | dysentary). Example C. rectus |
| Proteobacteria | Heliobacter | Example H pylori. Causes gastritis and gastric ulcers |
| Frimicutes | Clostridia | Multiple Species. Examples: C. Difficile, C. botulinum, |
| | | C. tetani. Cause a variety of serious conditions, from |
| | | colitis to paralysis |
| Frimicutes | Mycoplasmas | Multiple species. Examples: M. genitalium, M |
| | | pneumoniae. Ureaplasma species. Associated with |
| | | sexually transmited disease, infertility and infant |
| | | respiratory distress and brain hemorrhage. P1 antigen is |
| | | primary virulence factor, which is also expressed on |
| | | erthyrocytes. This can lead to autoantibody agglutination |
| Frimicutes | Bacillus | One of the most diverse genus from a speciation |
| | | perspective. Examples B. anthracis (anthrax), B. cereus |
| | | (food poisoning) |
| Frimicutes | Listeria | 15 identified species. Example: L. monocytogenes food |
| | | poisining. Less frequently seen disease manifestation = |
| | | listeriosis (sepsis and meningitis with a 20% fatality rate) |
| Frimicutes | Staphylococcus | Multiple species and sub. Example: S. aureus. Disease |
| | | can range form folliculitis to necrotizing pneumonia and |
| | | endocarditis. Commonly drug resistant (MRSA) |
| Frimicutes | Enterococcus | Multiple species. Cause UTI, Bacteremia, endocarditis, |
| | | diverticulitis, meningitis, prostatitis. Example: E. faecium. |
| | | Increasingly drug resistant (VRE) |
| Frimicutes | Lactobacillus | Often considered beneficial, but can cause septicemiva, |
| | | endocarditis, rheumatic vascular disease and dental |
| | | caries (particularly in immuno-compromised patients). |
| | | Example L. rhamnosus |
| Frimicutes | Streptococcus | Multiple species. Cause strep throat, pink eye, meningitis, |
| | | bacterial pneumonia, sepsis, endocarditis, erysipelas, |
| | | necrotizing fasciitis. Examples: S. pyogenes, S. pneumoniae, |
| | | S. sanguinis |
| Actinobacteria | Nocardia | Multiple species. Low virulence and generally infect the |
| | | immunocompromised only. Cause penumonia, |
| | | endocarditis, encephalitis, cellulitis. Example N. asteroids |
| Actinobacteria | Mycobacterium | Multiple species. Cause tuberculosis, leprosy. Examples |
| | | M. tuberculosis, M. leprae |
| Actinobacteria | Corynebacterium | Multiple species. Cause diptheria, colonizes prosthetics, |
| | | and can cause skin infections, endocarditis, pheumonitis. |
| | | Nosocomial |
| Actinobacteria | Actinomyces | Multiple species. Cause periodontal abscesses, |
| | | lympadenopathy, thoracic disease and abdominal abscess. |
| | | Example: Aggregatibacter actinomycetemcomitans |
| Chlamydia | Chlamydia | 4 species. Most common bacterial STD and can cause |
| | | blindness. Example Chlamydia trachomatis. |
| Spirochetes | Borrelia | 52 species. Cause Lyme disease and relapsing fever |
| | | (severe bacterimia). Example: Borrelia burgdorferi |
| Spirochetes | Leptospira | Multiple species (13 cause disease in humans). Cause |
| | | leptospirosis - symtpoms range from hedaches and fatigue |
| | | to meningitis, kidney failure and pulmonary hemorrhage. |
| Spirochetes | Treponema | Multiple species. Cuases syphilis Example: Treponema |
| | | pallidum. |
| Bacteroids | Bacteroides | Multiple species. Colonize the gut and cause infections |
| | | associated with surgery, appendicitis etc. Example: B. fragilis |

In certain embodiments, the infectious disease therapeutic is selected from a fungal polypeptide. In these embodiments, an aAPC comprises an erythroid cell (e.g., an enucleated erythroid cell) or an enucleated cell, comprising one or more exogenous polypeptides, wherein one of the one or more exogenous polypeptides comprises an antigenic fungal polypeptide, and the aAPC is administered to a subject to treat an infection with the fungus.

Exemplary fungal pathogens are shown below in Table 23.

TABLE 23
Fungal Pathogens

| Fungal | | |
|---|---|---|
| Group | Pathogen Species | Pathology example |
| Yeast | Candida albicans | Oral thrush, onychomycosis |
| Yeast | Candida glabrata | Vaginitis, esophageal candidisis |
| Yeast | Candida krusei | Invasive candidiasis, neutropenia |
| Yeast | Candida | Catheter and central line infections, biofims, UTI, |
| | parapsilosis | endocarditis, meningitis |
| Yeast | Candida tropicalis | Candidemia, oral thrush |
| Yeast | Rhodotorula | Sepsis, catheter infections, fungemia, meningitis, peritonitis |
| | mucilaginosa | |
| Yeast | Sporothrix | Sporotrichosis (lympocutaneous tissue, skin ulcerations) |
| | schenckii complex | |
| Yeast | Cryptococcus | Cryptococcal meningitis, pulmonary infection, osteomyelitis |
| | neoformans | |
| Yeast | Cryptococcus | Cryptococcal meningitis, pulmonary infection, osteomyelitis |
| | gattii | (more common in immunocompromised patients thant C. neoformans, |
| | | which is more prevelant in immunocompetent |
| | | patients) |
| Moulds | Alternaria | Allergic fungal rhinosinusitis, Keratitis, peritonitis, allergic |
| | alternata | respiratory disease |
| Moulds | Apophysomyces | Cutaneous and subcutaneous infection |
| | variabilis | |
| Moulds | Aspergillus | Chronic pulmonary aspergillosis, Asthma exacerbation with |
| | fumigatus | fingal sensitization, chronic invasive sinusitis, invasive and |
| | | disseminated aspergillosis |
| Moulds | Aspergillus flavus | Chronic cavity pulmonary aspergillosis, Cutaneous and |
| | | wound infection, endocarditis, pericarditis and CNS |
| | | infection, UTI |
| Moulds | Aspergillus niger | Otomycosis, SAFS, Allergic bronchopulmonary |
| | | aspergillosis, invasive pulmonary aspergillosis, disseminated |
| | | aspergillosis |
| Moulds | Aspergillus terreus | ABPA, aspergillus bronchitis, invasive aspergillosis, |
| | | disseminated aspergillosis |
| Moulds | Cladosphialophora | Chromoblastomycosis, mycetoma, phaehyphomycosis |
| | spp. | |
| Moulds | Exserohilum | Skin, corneal infection, invasive infection of sinus heart and |
| | | lung |
| Moulds | Fonsecaea | Chromoblastomycosis |
| | pedrosoi | |
| Moulds | Fusarium | Keratitis, Onychomycosis, endophthalmitis, skin infection, |
| | oxysporum | sinusitis, disseminated infection |
| Moulds | Fusarium solani | Keratitis, Onychomycosis, endophthalmitis, skin infection, |
| | | sinusitis, disseminated infection |
| Moulds | Lichtheimia | cutaneous, pulmonary, rhinocerebral CNS and disseminated |
| | corymbifera | infection (rare) |
| Moulds | Lichtheimia | cutaneous, pulmonary, rhinocerebral CNS and disseminated |
| | ramosa | infection (rare) |
| Moulds | Rhizopus | Mucormycosis |
| | microsporus | |
| Moulds | Stachybotrys | idiopathic pulmonary hemorrhage (infants) |
| Moulds | Trichophyton | Tiñea pedis, corporis, cruris, onychomycosis and |
| | interdigitale | occasionally Tinea capitis |
| Moulds | Trichophyton | Tinea of the groin, glabrous skin, feet, hands, and the nails. |
| | rubrum | Tinea cruris, tinea corporis, tinea pedis, tinea manuum, and |
| | | onychomycosis |
| Dimorphic | Histoplasma | Pneumonia, multi-organ failure |
| Fungi | capsulatum | |
| Dimorphic | Pneumocystis | Pneumonia (mostly in immune supressed patients) |
| Fungi | jirovecii | |
| Dimorphic | Paracoccidioides | Disseminated systemic infection |
| Fungi | brasiliensis | |
| Dimorphic | Penicillium | Disseminated infection with prominent skin lesions |
| Fungi | marneffei | (immunocompromised patients) |
| Dimorphic | Blastomyces | Skin infections, rearely disseminated |
| Fungi | | |
| Dimorphic | Coccidioides | Meningitis, disseminated disease, lung nodule, chronic |
| Fungi | | cavitary pulmonary coccidioidmycosis |

In certain embodiments, the infectious disease therapeutic is selected from a parasitic polypeptide. In these embodiments, an aAPC comprises an erythroid cell (e.g., an enucleated erythroid cell) or an enucleated cell, comprising one or more exogenous polypeptides, wherein one of the one or more exogenous polypeptides comprises an antigenic parasitic polypeptide, and the aAPC is administered to a subject to treat an infection with the parasite.

Exemplary parasitic pathogens are shown below in Table 24.

TABLE 24
Parasitic Pathogens

| Parasitic | | |
|---|---|---|
| Group | Pathogen Species | Disease/Pathology Example |
| Amoeba/ | Acanthamoeba | Acanthamoeba Infection |
| Protozoa | | |
| Amoeba/ | Acanthamoeba | Acanthamoeba Keratitis Infection |
| Protozoa | | |
| Amoeba/ | Trypanosoma brucei | African Sleeping Sickness (African trypanosomiasis) |
| Protozoa | | |
| Amoeba/ | Entamoeba histolytica | Amebiasis (Entamoeba histolytica Infection) |
| Protozoa | | |
| Amoeba/ | Trypanosoma cruzi | American Trypanosomiasis (Chagas Disease) |
| Protozoa | | |
| Amoeba/ | Balantidium coli | Balantidiasis (Balantidium Infection) |
| Protozoa | | |
| Amoeba/ | Balamuthia | Balamuthia (Granulomatous Amebic Encephalitis |
| Protozoa | mandrillaris | (GAE)) |
| Amoeba/ | Cryptosporidium | Cryptosporidiosis (Cryptosporidium Infection) |
| Protozoa | | |
| Amoeba/ | Cyclospora | Cyclosporiasis (Cyclospora Infection) |
| Protozoa | | |
| Amoeba/ | Taenia solium | Cysticercosis (Neurocysticercosis) |
| Protozoa | | |
| Amoeba/ | Cystoisospora belli | Cystoisospora Infection (Cystoisosporiasis) formerly |
| Protozoa | | Isospora Infection |
| Amoeba/ | | |
| Protozoa | Dientamoeba fragilis | Dientamoeba fragilis Infection |
| Amoeba/ | Entamoeba histolytica | Entamoeba histolytica Infection (Amebiasis) |
| Protozoa | | |
| Amoeba/ | Giardia | Giardiasis (Giardia Infection) |
| Protozoa | intestinalis, Giardia | |
| | lamblia, or Giardia | |
| | duodenalis | |
| Amoeba/ | Leishmania | Kala-azar (Leishmaniasis, Leishmania Infection) |
| Protozoa | | |
| Amoeba/ | Acanthamoeba | Keratitis (Acanthamoeba Infection) |
| Protozoa | | |
| Amoeba/ | Multiple species. | Malaria (Plasmodium Infection) |
| Protozoa | Examples: P. vivax and | |
| | P. ovale | |
| Amoeba/ | Naegleria fowleri | Naegleria Infection |
| Protozoa | “brain-eating amoeba” | |
| Amoeba/ | Sappinia ameoba | Sappinia (amebic encephalitis) |
| Protozoa | | |
| Amoeba/ | Sarcocystis | Sarcocystosis (Sarcocystosis Infection) |
| Protozoa | | |
| Amoeba/ | Toxoplasma gondii | Toxoplasmosis (Toxoplasma Infection) - protazoa |
| Protozoa | | |
| Amoeba/ | Trichomonas vaginalis | Trichomoniasis (Trichomonas Infection) - protozoa |
| Protozoa | | |
| Amoeba/ | Trypanosoma brucei | Trypanosomiasis, African (African Sleeping Sickness, |
| Protozoa | | Sleeping Sickness) - protozoa |
| Amoeba/ | Babesia (various | Babesiosis (Babesia Infection) - similar lifecycle to |
| Protozoa | species) | malaria, taken up by RBCs |
| Arthropod | Cimex | Bed Bugs |
| | lectularius and C. hemipterus | |
| Arthropod | Pediculus humanus | Body Lice Infestation (Pediculosis) |
| | humanus | |
| Arthropod | Phthirus pubis | Crabs (Pubic Lice) |
| Arthropod | Pediculus humanus | Head Lice Infestation (Pediculosis) |
| | capitis | |
| Arthropod | Sarcoptes | Mite Infestation (Scabies) |
| | scabiei var. hominis | |
| Arthropod | infection of a fly larva. | Myiasis |
| | Examples: Dermatobia | |
| | hominis | |
| Other | group of obligate | Microsporidiosis (Microsporidia Infection) |
| | intracellular parasitic | |
| | fungi. Examples: M. africanum, | |
| | Nosema | |
| | ocularum | |
| Other | Pneumocystis jirovecii | Pneumocystis jirovecii Pneumonia |
| | (considered a parasitic | |
| | fungi) | |
| Protist | Blastocystis hominis | Blastocystis hominis Infection |
| Worm | Echinococcus | Alveolar Echinococcosis (Echinococcosis, Hydatid |
| | granulosus | Disease) |
| Worm | Hookworm (various | Ancylostomiasis (Hookworm) |
| | species) | |
| Worm | Angiostrongylus | Angio strongyliasis (Angiostrongylus Infection) |
| | (various species) | |
| | nematode | |
| Worm | anisakid nematodes | Anisakiasis (Anisakis Infection, Pseudoterranova |
| | | Infection) |
| Worm | Ascaris lumbricoides | Ascariasis (Ascaris Infection, Intestinal Roundworms) |
| Worm | B. procyonis | Baylisascariasis (Baylisascaris Infection, Raccoon |
| | | Roundworm) |
| Worm | S. mansoni | Bilharzia (Schistosomiasis) |
| Worm | Capillaria | Capillariasis (Capillaria Infection) |
| | hepatica and Capillaria | |
| | philippinensis | |
| Worm | Various species, | Cercarial Dermatitis (Swimmer's Itch) |
| | example: | |
| | Austrobilharzia | |
| | variglandis | |
| Worm | Clonorchis liver fluke | Clonorchiasis (Clonorchis Infection) |
| Worm | Diphyllobothrium | Diphyllobothriasis (Diphyllobothrium Infection) |
| | latum (tapeworm) | |
| Worm | Dipylidiu tapeworm | Dipylidium caninum Infection (dog or cat tapeworm |
| | | infection) |
| Worm | Dirofilaria roundworms | Dirofilariasis (Dirofilaria Infection) |
| Worm | Guinea worm | Dracunculiasis (Guinea Worm Disease) |
| Worm | Echinococcus | Echinococcosis (Cystic, Alveolar Hydatid Disease) |
| | granulosus & | |
| | Echinococcus | |
| | multilocularis | |
| Worm | Wuchereria | Elephantiasis (Filariasis, Lymphatic Filariasis) |
| | bancrofti, Brugia malayi | |
| | and Brugia timori | |
| Worm | Enterobius | Enterobiasis (Pinworm Infection) |
| | vermicularis | |
| Worm | Fasciola hepatica | Fascioliasis (Fasciola (liver fluke) Infection) |
| Worm | Fasciolopsis buski | Fasciolopsiasis (Fasciolopsis Infection) |
| Worm | Gnathostoma: several | Gnathostomiasis (Gnathostoma Infection) |
| | species of parasitic | |
| | nematodes | |
| Worm | trematode Heterophyes | Heterophyiasis (Heterophyes Infection) |
| | heterophyes | |
| Worm | Multiple species. | Hookworm Infection, Human |
| | Example: L: Filariform | |
| Worm | Multiple species. | Hookworm Infection, Zoonotic (Ancylostomiasis, |
| | Example: Ancylostoma | Cutaneous Larva Migrans [CLM]) |
| | brazilense, A. caninum, | |
| | A. ceylanicum | |
| Worm | H. nana (dwarf | Hymenolepiasis (Hymenolepis Infection) |
| | tapeworm) | |
| Worm | Various species, | Intestinal Roundworms (Ascariasis, Ascaris Infection) |
| | example: A. lumbricoides | |
| Worm | Loa boa: parasitic | Loiasis (Loa boa Infection) |
| | worm | |
| Worm | Taenia solium | Neurocysticercosis (Cysticercosis) |
| Worm | Toxocara canis and | Ocular Larva Migrans (Toxocariasis, Toxocara Infection, |
| | Toxocara cati | Visceral Larva Migrans) |
| Worm | Onchocerca volvulus | Onchocerciasis (River Blindness) |
| Worm | Opisthorchis (liver | Opisthorchiasis (Opisthorchis Infection) |
| | fluke). Example: O. felineus | |
| Worm | Paragonimus (lung | Paragonimiasis (Paragonimus Infection) |
| | fluke) | |
| Worm | anisakid nematodes | Pseudoterranova Infection (Anisakiasis, Anisakis |
| | | Infection) |
| Worm | Strongyloides | Strongyloidiasis (Strongyloides Infection) |
| | nematodes. Example: | |
| | S. stercoralis | |
| Worm | Taenia saginata | Taeniasis (Taenia Infection, Tapeworm Infection) |
| Worm | Trichinella | Trichinellosis (Trichinosis) |
| Worm | T. trichiura | Trichuriasis (Whipworm Infection, Trichuris Infection) |

In some embodiments, the infectious diseases is multi-drug resistant staph. In another embodiments, the infectious diseases is pseudomonas. In another embodiment, the infectious disease is a nosocomial infections (i.e. any systemic or localized conditions that result from the reaction by an infectious agent or toxin) that are difficult to treat, for example infections caused by Clostridium difficile.

Other

Other diseases and disorders are contemplated for treatment by the aAPCs of the present disclosure. Examples include, but are not limited to cardiovascular diseases and immune diseases.

### Subjects

The methods described herein are intended for use with any subject that may experience the benefits of these methods. Thus, “subjects,” “patients,” and “individuals” (used interchangeably) include humans as well as non-human subjects, particularly domesticated animals.

In some embodiments, the subject and/or animal is a mammal, e g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal. In some embodiments, the subject and/or animal is a human. In some embodiments, the human is a pediatric human In other embodiments, the human is an adult human. In other embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the disclosure pertains to veterinary use. In a specific embodiment, the non-human animal is a household pet. In another specific embodiment, the non-human animal is a livestock animal In certain embodiments, the subject is a human cancer patient that cannot receive chemotherapy, e.g. the patient is unresponsive to chemotherapy or too ill to have a suitable therapeutic window for chemotherapy (e.g. experiencing too many dose- or regimen-limiting side effects). In certain embodiments, the subject is a human cancer patient having advanced and/or metastatic disease.

In some embodiments, the subject is selected for treatment with an aAPC comprising an erythroid cell presenting (e.g. comprising on the cell surface) one or more exogenous polypeptides of the present disclosure. In some embodiments, the subject is selected for treatment of cancer with an aAPC comprising an erythroid cell presenting (e.g. comprising on the cell surface) one or more exogenous polypeptides of the present disclosure. In some embodiments, the subject is selected for treatment of an autoimmune disease with an aAPC comprising an erythroid cell presenting (e.g. comprising on the cell surface) one or more exogenous polypeptides of the present disclosure. In some embodiments, the subject is selected for treatment of an infectious disease with an aAPC comprising an erythroid cell presenting (e.g. comprising on the cell surface) one or more exogenous polypeptides of the present disclosure.

In some embodiments of the above aspects and embodiments, the erythroid cell is an enucleated erythroid cell. In some embodiments of the above aspects and embodiments, the erythroid cell is a nucleated erythroid cell.

### V. Pharmaceutical Compositions

The present disclosure encompasses the preparation and use of pharmaceutical compositions comprising an aAPC of the disclosure as an active ingredient. Such a pharmaceutical composition may consist of the active ingredient alone, as a combination of at least one active ingredient (e.g., an effective dose of an aAPC) in a form suitable for administration to a subject, or the pharmaceutical composition may comprise the active ingredient and one or more pharmaceutically acceptable carriers, one or more additional (active and/or inactive) ingredients, or some combination of these.

In some embodiments, the disclosure features a pharmaceutical composition comprising a plurality of the aAPCs described herein, and a pharmaceutical carrier. In other embodiments, the disclosure features a pharmaceutical composition comprising a population of aAPCs as described herein, and a pharmaceutical carrier. It will be understood that any single aAPCs, plurality of aAPCs, or population of aAPCs as described elsewhere herein may be present in a pharmaceutical composition of the invention.

In some embodiments, the pharmaceutical compositions provided herein comprise engineered (i.e. modified) erythroid cells and unmodified erythroid cells. For example, a single unit dose of aAPCs can comprise, in various embodiments, about, at least, or no more than 10%, 20%, 30%, 40%, 50%, 60%, 70%, 75%, 80%), 85%, 90%, 95%, or 99% engineered erythroid cells, wherein the remaining erythroid cells in the composition are not engineered.

In some embodiments, the pharmaceutical compositions provided herein comprise aAPCs comprising engineered enucleated erythroid cells and nucleated erythroid cells. For example, a single unit dose of engineered erythroid cells (e.g., enucleated and nucleated erythroid cells) can comprise, in various embodiments, about, or at least 10%, 20%, 30%, 40%, 50%, 60%, 70%, 75%, 80%, 85%, 90%, 91%, 92%, 93%, 94%, 95%, 96%, 97%, 98%, or 99% enucleated erythroid cells, wherein the remaining erythroid cells in the composition are nucleated.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The administration of the pharmaceutical compositions may be carried out in any convenient manner, including by aerosol inhalation, injection, ingestion, transfusion, implantation or transplantation. The compositions of the present disclosure may be administered to a patient subcutaneously, intradermally, intramuscularly, by intravenous (i.v.) injection, or intraperitoneally. The pharmaceutical compositions may be injected directly into a tumor or lymph node.

As used herein, the term “pharmaceutically acceptable carrier” means a chemical composition with which the active ingredient may be combined and which, following the combination, can be used to administer the active ingredient to a subject.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs, birds including commercially relevant birds such as chickens, ducks, geese, and turkeys, fish including farm-raised fish and aquarium fish, and crustaceans such as farm-raised shellfish.

Pharmaceutical compositions that are useful in the methods of the disclosure may be prepared, packaged, or sold in formulations suitable for oral, rectal, vaginal, parenteral, topical, pulmonary, intranasal, intra-lesional, buccal, ophthalmic, intravenous, intra-organ or another route of administration. Other contemplated formulations include projected nanoparticles, liposomal preparations, resealed erythrocytes containing the active ingredient, and immunologically-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents. Particularly contemplated additional agents include anti-emetics and scavengers such as cyanide and cyanate scavengers and AZT, protease inhibitors, reverse transcriptase inhibitors, interleukin-2, interferons, cytokines, and the like.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The aAPC of the disclosure and/or T cells expanded using the aAPC, can be administered to an animal, preferably a human. When the T cells expanded using an aAPC of the disclosure are administered, the amount of cells administered can range from about 1 million cells to about 300 billion. Where the aAPCs themselves are administered, either with or without T cells expanded thereby, they can be administered in an amount ranging from about 100,000 to about one billion cells wherein the cells are infused into the animal, preferably, a human patient in need thereof. While the precise dosage administered will vary depending upon any number of factors, including but not limited to, the type of animal and type of disease state being treated, the age of the animal and the route of administration.

The aAPC may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

An aAPC (or cells expanded thereby) may be co-administered with the various other compounds (cytokines, chemotherapeutic and/or antiviral drugs, among many others). Alternatively, the compound(s) may be administered an hour, a day, a week, a month, or even more, in advance of the aAPC (or cells expanded thereby), or any permutation thereof. Further, the compound(s) may be administered an hour, a day, a week, or even more, after administration of aAPC (or cells expanded thereby), or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds and the aAPC (or cells expanded thereby), and the like.

Further, it would be appreciated by one skilled in the art, based upon the disclosure provided herein, that where the aAPC is to be administered to a mammal, the cells are treated so that they are in a “state of no growth”; that is, the cells are incapable of dividing when administered to a mammal. As disclosed elsewhere herein, the cells can be irradiated to render them incapable of growth or division once administered into a mammal. Other methods, including haptenization (e.g., using dinitrophenyl and other compounds), are known in the art for rendering cells to be administered, especially to a human, incapable of growth, and these methods are not discussed further herein. Moreover, the safety of administration of aAPC that have been rendered incapable of dividing in vivo has been established in Phase I clinical trials using aAPC transfected with plasmid vectors encoding some of the molecules discussed herein.

### Combination Therapies

In some embodiments, the disclosure provides methods that further comprise administering an additional agent to a subject. In some embodiments, the disclosure pertains to co-administration and/or co-formulation.

In some embodiments, administration of the aAPC acts synergistically when co-administered with another agent and is administered at doses that are lower than the doses commonly employed when such agents are used as monotherapy.

In some embodiments, inclusive of, without limitation, cancer applications, the present disclosure pertains to chemotherapeutic agents as additional agents. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores), aclacinomy sins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel, and TAXOTERE doxetaxel; chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB); inhibitors of PKC-α., Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation.

Some human tumors can be eliminated by a patient's immune system. For example, administration of a monoclonal antibody targeted to an immune “checkpoint” molecule can lead to complete response and tumor remission. A mode of action of such antibodies is through inhibition of an immune regulatory molecule that the tumors have co-opted as protection from an anti-tumor immune response. By inhibiting these “checkpoint” molecules (e.g., with an antagonistic antibody), a patient's CD8+ T cells may be allowed to proliferate and destroy tumor cells.

For example, administration of a monoclonal antibody targeted to by way of example, without limitation, CTLA-4 or PD-1 can lead to complete response and tumor remission. The mode of action of such antibodies is through inhibition of CTLA-4 or PD-1 that the tumors have co-opted as protection from an anti-tumor immune response. By inhibiting these “checkpoint” molecules (e.g., with an antagonistic antibody), a patient's CD8+ T cells may be allowed to proliferate and destroy tumor cells.

Thus, the aAPCs comprising an enucleated cell or erythroid cell presenting (e.g. comprising on the cell surface) one or more exogenous polypeptides provided herein can be used in combination with one or more blocking antibodies targeted to an immune “checkpoint” molecule. For instance, in some embodiments, the compositions provided herein can be used in combination with one or more blocking antibodies targeted to a molecule such as CTLA-4 or PD-1. For example, the compositions provided herein may be used in combination with an agent that blocks, reduces and/or inhibits PD-1 and PD-L1 or PD-L2 and/or the binding of PD-1 with PD-L1 or PD-L2 (by way of non-limiting example, one or more of nivolumab (ONO-4538/BMS-936558, MDX1106, OPDIVO, BRISTOL MYERS SQUIBB), pembrolizumab (KEYTRUDA, Merck), pidilizumab (CT-011, CURE TECH), MK-3475 (MERCK), BMS 936559 (BRISTOL MYERS SQUIBB), MPDL328OA (ROCHE)). In an embodiment, the compositions provided herein may be used in combination with an agent that blocks, reduces and/or inhibits the activity of CTLA-4 and/or the binding of CTLA-4 with one or more receptors (e.g. CD80, CD86, AP2M1, SHP-2, and PPP2R5A). For instance, in some embodiments, the immune-modulating agent is an antibody such as, by way of non-limitation, ipilimumab (MDX-010, MDX-101, Yervoy, BMS) and/or tremelimumab (Pfizer). Blocking antibodies against these molecules can be obtained from, for example, Bristol Myers Squibb (New York, N.Y.), Merck (Kenilworth, N.J.), MedImmune (Gaithersburg, Md.), and Pfizer (New York, N.Y.).

Further, the aAPC compositions provided herein can be used in combination with one or more blocking antibodies targeted to an immune “checkpoint” molecule such as for example, BTLA, HVEM, TIM3, GALS, LAG3, VISTA, KIR, 2B4, CD160 (also referred to as BY55), CGEN-15049, CHK 1 and CHK2 kinases, A2aR, CEACAM (e.g., CEACAM-1, CEACAM-3 and/or CEACAM-5), GITR, GITRL, galectin-9, CD244, CD160, TIGIT, SIRPα, ICOS, CD172a, and TMIGD2 and various B-7 family ligands (including, but are not limited to, B7-1, B7-2, B7-DC, B7-H1, B7-H2, B7-H3, B7-H4, B7-H5, B7-H6 and B7-H7).

In some embodiments of the above aspects and embodiments, the erythroid cell is an enucleated erythroid cell. In some embodiments of the above aspects and embodiments, the erythroid cell is a nucleated erythroid cell.

In some embodiments, the disclosure features a pharmaceutical composition comprising a plurality of the engineered erythroid cells described herein, and a pharmaceutical carrier. In other embodiments, the disclosure features a pharmaceutical composition comprising a population of engineered erythroid cells as described herein, and a pharmaceutical carrier. It will be understood that any single engineered erythroid cell, plurality of engineered erythroid cells, or population of engineered erythroid cells as described elsewhere herein may be present in a pharmaceutical composition of the invention.

In some embodiments, the pharmaceutical compositions provided herein comprise engineered (i.e. modified) erythroid cells and unmodified erythroid cells. For example, a single unit dose of erythroid cells (e.g., modified and unmodified erythroid cells) can comprise, in various embodiments, about, at least, or no more than 10%, 20%, 30%, 40%, 50%, 60%, 70%, 75%, 80%), 85%, 90%, 95%, or 99% engineered erythroid cells, wherein the remaining erythroid cells in the composition are not engineered.

In some embodiments, the pharmaceutical compositions provided herein comprise engineered enucleated erythroid cells and nucleated erythroid cells. For example, a single unit dose of engineered erythroid cells (e.g., enucleated and nucleated erythroid cells) can comprise, in various embodiments, about, or at least 10%, 20%, 30%, 40%, 50%, 60%, 70%, 75%, 80%, 85%, 90%, 91%, 92%, 93%, 94%, 95%, 96%, 97%, 98%, or 99% enucleated erythroid cells, wherein the remaining erythroid cells in the composition are nucleated.

### VI. Kits

The disclosure includes various kits which comprise an aAPC of the disclosure, a nucleic acid encoding various proteins, an antibody that specifically binds to a costimulatory molecule on the surface of a T cell, and/or a nucleic acid encoding the antibody of the disclosure, an antigen, or an cytokine, an applicator, and instructional materials which describe use of the kit to perform the methods of the disclosure. Although exemplary kits are described below, the contents of other useful kits will be apparent to the skilled artisan in light of the present disclosure. Each of these kits is included within the disclosure.

The disclosure includes a kit for specifically inducing proliferation of a T cell expressing a known co-stimulatory molecule. This is because contacting the T cell with an aAPC, specifically induces proliferation of the T cell. The kit is used pursuant to the methods disclosed in the disclosure. Briefly, the kit may be used to administer an aAPC of the disclosure to a T cell expressing at least one costimulatory molecule. This is because, as more fully disclosed elsewhere herein, the data disclosed herein demonstrate that contacting a T cell with an aAPC comprising a costimulatory ligand that specifically binds with the cognate costimulatory molecule present on the T cell, mediates stimulation and activation of the T cell. Further, the T cells produced using this kit can be administered to an animal to achieve therapeutic results.

The kit further comprises an applicator useful for administering the aAPC to the T cells. The particular applicator included in the kit will depend on, e.g., the method used to administer the aAPC, as well as the T cells expanded by the aAPC, and such applicators are well-known in the art and may include, among other things, a pipette, a syringe, a dropper, and the like. Moreover, the kit comprises an instructional material for the use of the kit. These instructions simply embody the disclosure provided herein.

The kit includes a pharmaceutically-acceptable carrier. The composition is provided in an appropriate amount as set forth elsewhere herein. Further, the route of administration and the frequency of administration are as previously set forth elsewhere herein.

The kit encompasses an aAPC comprising a wide plethora of molecules, such as, but not limited to, those set forth herein. However, the skilled artisan armed with the teachings provided herein, would readily appreciate that the disclosure is in no way limited to these, or any other, combination of molecules. Rather, the combinations set forth herein are for illustrative purposes and they in no way limit the combinations encompassed by the present disclosure. Further, the kit comprises a kit where each molecule to be transduced into the aAPC is provided as an isolated nucleic acid encoding a molecule, a vector comprising a nucleic acid encoding a molecule, and any combination thereof, including where at least two molecules are encoded by a contiguous nucleic acid and/or are encoded by the same vector.

All publications and patent applications cited in this specification are herein incorporated by reference in their entirety for all purposes as if each individual publication or patent application were specifically and individually indicated to be incorporated by reference for all purposes. The publications discussed herein are provided solely for their disclosure prior to the filing date of the present application. Nothing herein is to be construed as an admission that the inventors described herein are not entitled to antedate such disclosure by virtue of prior disclosure or for any other reason.

### EXAMPLES

### Example 1. Generation of Erythroid Cells Genetically Engineered to Express a MOG-MHCII-GPA Fusion Protein

### Results

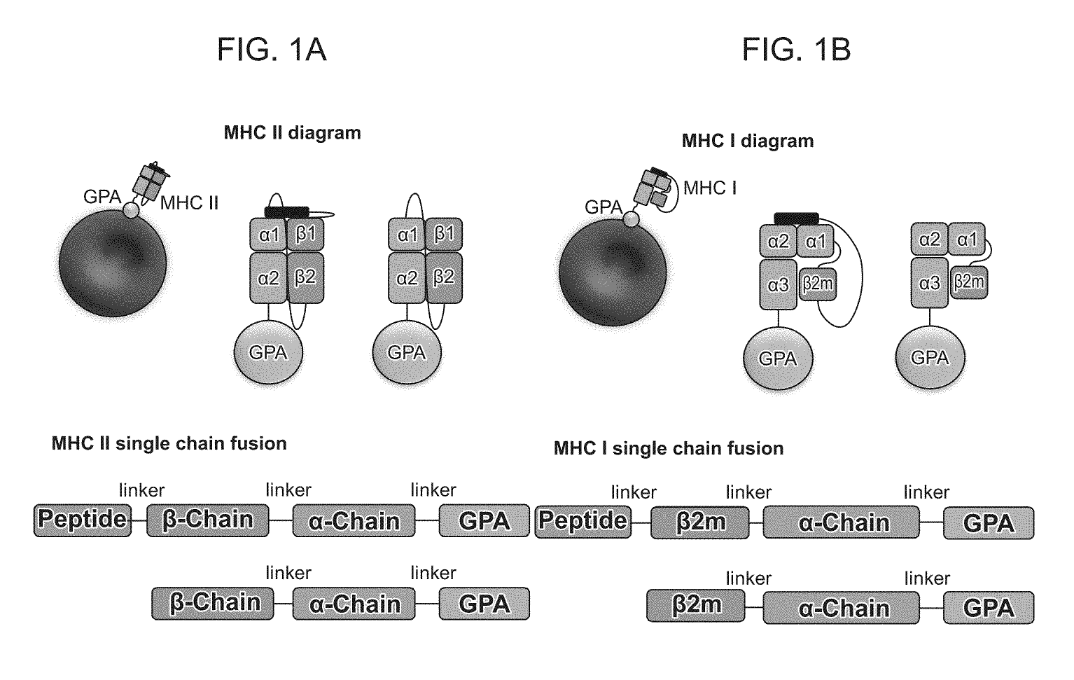

Erythroid cells are transduced to express a fusion protein comprising an exogenous antigenic peptide, myelin oligodendrocyte glycoprotein (MOG), fused to an exogenous antigen presenting polypeptide, specifically MHCII, fused to the GPA transmembrane domain (GPA), which serves as a membrane anchor (MOG-MHCII-GPA). FIG. 1A shows a schematic of the design for expressing MOG peptide and MHCII as a single chain fusion, where the exogenous peptide (MOG) is linked to the MHCII β-chain, which is linked to the MHCII α-chain, which is linked to the GPA membrane anchor. Cell culture and transduction is performed as described in the “Methods” section below to yield erythroid cells expressing MOG presented by MHCII on the cell surface, anchored with a GPA transmembrane domain.

Binding of an allophycocyanin (APC)-labelled or Phycoerythrin (PE)-labelled anti-MOG antibody is used to validate expression of the antigenic peptides in the engineered erythroid cells. Binding of an APC-labelled or PE-labelled anti-MHCII antibody is used to validate expression of the MHCII antigen presenting peptide in the engineered erythroid cells.

### Methods

Production of Lentiviral Vector

The gene encoding the MOG-MHCII-GPA fusion protein is cloned into the multiple cloning site of lentivirus vector pCDH with the MSCV promoter sequence from System Biosciences. Lentivirus is produced in 293T cells by transfecting the cells with pPACKH1 (System Biosciences) and pCDH lentivirus vector containing the MOG-MHCII-GPA gene. Cells are then placed in fresh culturing medium. The virus supernatant is collected 48 hours post-medium change by centrifugation at 1,500 rpm for 5 minutes. The supernatant is collected and frozen in aliquots at −80° C.

Expansion and Differentiation of Erythroid Cells

Human CD34+ cells derived from mobilized peripheral blood cells from normal human donors are purchased frozen from AllCells Inc. The expansion/differentiation procedure comprises 3 stages. In the first stage, thawed CD34+ erythroid precursors are cultured in Iscove's MDM medium comprising recombinant human insulin, human transferrin, recombinant human recombinant human stem cell factor, and recombinant human interleukin 3. In the second stage, erythroid cells are cultured in Iscove's MDM medium supplemented with bovine serum albumin, recombinant human insulin, human transferrin, human recombinant stem cell factor, human recombinant erythropoietin, and L-glutamine. In the third stage, erythroid cells are cultured in Iscove's MDM medium supplemented with human transferrin, recombinant human insulin, human recombinant erythropoietin, and heparin. The cultures are maintained at 37° C. in 5% CO2 incubator.

Transduction of Erythroid Precursor Cells

Erythroid precursor cells are transduced during step 1 of the culture process described above. Erythroid cells in culturing medium are combined with lentiviral supernatant and polybrene. Infection is achieved by spinoculation, spinning the plate at 2000 rpm for 90 minutes at room temperature. After spinoculation, the cells are incubated at 37° C. overnight.

Antibody Binding

Binding of an APC-labelled or PE-labelled anti-MHCII antibody (e.g. Anti-Human HLA-DR (HLA Class II) Monoclonal Antibody, Allophycocyanin Conjugated, Clone Immu-357 from Beckman Coulter Life Sciences) is used to validate expression of the MHCII antigen presenting peptide in the engineered erythroid cells. Binding of the antibody is measured by flow cytometry for APC fluorescence or PE fluorescence. A gate is set based on stained untransduced cells.

### Example 2. Generation and In Vitro Validation of Erythroid Cells Genetically Engineered to Co-Express a MOG-MHCII-GPA Fusion Protein and a Coinhibitor Polypeptide

Results

Erythroid cells are transduced to express a fusion protein comprising an exogenous antigenic peptide, MOG, fused to an exogenous antigen presenting polypeptide, MHCII, fused to the GPA transmembrane domain (GPA) (MOG-MHCII-GPA), as described in Example 1. The erythroid cells are co-transduced to additionally express an exogenous coinhibitory peptide, PD-L1. Cell culture and transduction is performed as described in the “Methods” section below to yield erythroid cells expressing MOG presented by MHCII on the surface, anchored with a GPA transmembrane domain, and co-expressing PD-L1.

Binding of an APC-labelled or PE-labelled anti-MHCII antibody is used to validate expression of the MHCII antigen presenting peptide in the engineered erythroid cells, also as described in Example 1. Binding of an APC-labelled or PE-labelled anti-PD-L1 antibody is used to validate expression of PD-L1 in the engineered erythroid cells.

The effect of the engineered erythroid cells (MOG-MHCII-GPA and PD-L1) is assessed by determining one or more effects on suppressing a T cell, including (1) inhibition of T cell activity, (2) inhibition of T cell proliferation, and/or (3) induction of apoptosis of a T cell.

### Methods

Production of Lentiviral Vector

The genes for MOG-MHCII-GPA fusion protein and PD-L1 are constructed. Genes encoding the proteins are cloned into the multiple cloning site of lentivirus vector pCDH with the MSCV promoter sequence from System Biosciences, such that one vector comprises the genes for both exogenous proteins. Lentivirus is produced in 293T cells by transfecting the cells with pPACKH1 (System Biosciences) and pCDH lentivirus vector containing genes for MOG-MHCII-GPA and PD-L1. Cells are placed in fresh culturing medium. The virus supernatant is collected 48 hours post-medium change by centrifugation at 1,500 rpm for 5 minutes. The supernatant is collected and frozen in aliquots at −80° C.

Transduction of Erythroid Precursor Cells

Expansion and differentiation of erythroid cells is performed according to Example 1, with the lentiviral vectors described above. Erythroid precursor cells are transduced during step 1 of the culture process. Erythroid cells in culturing medium are combined with lentiviral supernatant and polybrene. Infection is achieved by spinoculation, spinning the plate at 2000 rpm for 90 minutes at room temperature. After spinoculation, the cells are incubated at 37° C. overnight.

Antibody Binding

Binding of an APC-labelled or PE-labelled anti-MHCII antibody to validate expression of the MHCII antigen presenting peptide in the engineered erythroid cells is carried out as described in Example 1. Binding of APC-labelled or PE-labelled anti-PD-L1 antibody (e.g. APC anti-human CD274 (B7-H1, PD-L1) Antibody (Biolegend)) is used to validate expression of PD-L1 in the engineered erythroid cells. Binding of the antibodies is measured by flow cytometry for APC fluorescence or PE fluorescence. A gate is set based on stained untransduced cells.

Functional Validation Assays

Effects of the engineered erythroid cells coexpressing MOG-MHCII-GPA and PD-L1 on T cell suppression are assessed by determining one or more of (1) inhibition of T cell activity, (2) inhibition of T cell proliferation, and (3) induction of apoptosis of a T cell. Inhibition of T cell activity is determined, for example, by cytokine analysis of supernatants with commercially available ELISA kits for human IL-2, IFN-γ, and IL-10 (R&D Systems). For example, after treatment with the engineered erythroid cells, detection of inhibition of IL-2 secretion by activated T cells, would indicate an antiproliferative effect of engineered erythroid cells coexpressing MOG-MHCII-GPA and PD-L1. Inhibition of T cell proliferation is assayed, for example, by labelling cells with the fluorescent dye 5,6-carboxyfluorescein diacetate succinimidyl ester (CFSE). Those cells that proliferate in response to the engineered erythroid cell show a reduction in CFSE fluorescence intensity, which is measured directly by flow cytometry. Alternatively, radioactive thymidine incorporation can be used to assess the rate of growth of the T cells stimulated with erythroid cells engineered to co-express a MOG-MHCII-GPA fusion protein and a PD-L1 coinhibitor polypeptide. Induction of apoptosis of a T cell by the engineered erythroid cells coexpressing MOG-MHCII-GPA and PD-L1 is assayed using, for example, fluorochrome-conjugated annexin V staining. BATF is a bZIP transcription factor that plays an important role in regulating differentiation and function in many lymphocyte lineages. In the CD8+ T cell lineage, increased expression of BATF in exhausted CD8+ T cells suppresses their effector function. BAFT has been shown to be a central regulator of early effector CD8+ T cell differentiation. Accordingly, assessment of the expression of transcription factors, such as Basic leucine zipper transcription factor ATF-like (BATF), can be used to determine the effect of MOG-MHCII-GPA and PD-L1 on T cell suppression.

### Example 3. Generation and In Vitro Validation of Erythroid Cells Genetically Engineered to Co-Express a MOG-MHCII-GPA Fusion Protein and a Treg Expansion Polypeptide

### Results

Erythroid cells are transduced to express a fusion protein comprising an exogenous antigenic peptide, MOG, fused to an exogenous antigen presenting polypeptide, MHCII, fused to the GPA transmembrane domain (GPA) (MOG-MHCII-GPA), as described in Example 1. The erythroid cells are also transduced to additionally express an exogenous Treg expansion polypeptide, a TNFR2-specific TNFα polypeptide. Cell culture and transduction is performed as described in the “Methods” section below to yield erythroid cells expressing MOG presented by MHCII on the surface, anchored with a GPA transmembrane domain and co-expressing a TNFR2-specific TNFα polypeptide.

Binding of an APC-labelled or PE-labelled anti-TNFα is used to validate expression of the antigenic peptides in the engineered erythroid cells. Binding of an APC-labelled or PE-labelled anti-MHCII antibody is used to validate expression of the MHCII antigen presenting peptide in the engineered erythroid cells. Functional activity of the effect of the engineered erythroid cells (MOG-MHCII-GPA and TNFR2 specific TNFα) is assessed using a human CD4+ T-cell-based proliferative assay, as described in Example 2.

### Methods

Production of Lentiviral Vector

The genes for MOG-MHCII-GPA fusion protein and TNFR2 specific TNFα are constructed. Genes encoding the proteins are cloned into the multiple cloning site of lentivirus vector pCDH with the MSCV promoter sequence from System Biosciences, such that one vector comprises the genes for both exogenous proteins. Lentivirus is produced in 293T cells by transfecting the cells with pPACKH1 (System Biosciences) and pCDH lentivirus vector containing genes for MOG-MHCII-GPA and TNFR2-specific TNFα. Cells are placed in fresh culturing medium. The virus supernatant is collected 48 hours post-medium change by centrifugation at 1,500 rpm for 5 minutes. The supernatant is collected and frozen in aliquots at −80° C.

Transduction of Erythroid Precursor Cells

Expansion and differentiation of erythroid cells is performed according to Example 1, with the lentiviral vector described above. Erythroid precursor cells are transduced during step 1 of the culture process. Erythroid cells in culturing medium are combined with lentiviral supernatant and polybrene. Infection is achieved by spinoculation, spinning the plate at 2000 rpm for 90 minutes at room temperature. After spinoculation, the cells are incubated at 37° C. overnight.

Antibody Binding

Binding of an APC-labelled or PE-labelled anti-MHCII antibody to validate expression of the MHCII antigen presenting peptide in the engineered erythroid cells is carried out, also as described in Example 1. Binding of an APC-labelled or PE-labelled TNFα antibody is used to validate expression of TNFR2-specific TNFα mutant in the engineered erythroid cells. Binding of any one of the antibodies is measured by flow cytometry for APC fluorescence or PE fluorescence. A gate is set based on stained untransduced cells.

Functional Validation Assays

Functional activity is assessed using a human CD4+ T-cell-based proliferative assay. Cells are labeled with the fluorescent dye 5,6-carboxyfluorescein diacetate succinimidyl ester (CFSE). Those cells that proliferate in response to the engineered erythrocytes show a reduction in CFSE fluorescence intensity, which is measured directly by flow cytometry. Alternatively radioactive thymidine incorporation can be used to assess the rate of growth of the T cells stimulated with erythroid cells engineered to co-express a MOG-MHCII-GPA fusion protein and a CD25 specific-IL2 Treg expansion polypeptide.

Functional activity is also assessed using an in vitro Treg suppression assay. Such an assay is described in Collinson and Vignali (Methods Mol Biol. 2011; 707: 21-37, incorporated by reference in its entirety herein).

### Example 4. Activity of Enucleated Erythroid Cells Co-Expressing a MOG-MHCII-GPA Fusion Protein and a Coinhibitor Polypeptide or a Treg Expansion Polypeptide in a Murine EAE Model

Experimental autoimmune encephalomyelitis (EAE) is the model most commonly used to study efficacy of potential drugs for treatment of multiple sclerosis (MS). Because of its many similarities to MS, EAE is used to study pathogenesis of autoimmunity, CNS inflammation, demyelination, cell trafficking and tolerance induction. EAE is characterized by paralysis (in some models the paralysis is relapsing-remitting), CNS inflammation and demyelination. Hooke Kits™ for EAE Induction in C57BL/6 Mice (Hooke Laboratories) are used to induce EAE in female C57BL/6 mice. Using this method, EAE is induced in C57BL/6 mice by immunization with an emulsion of MOG35-55 or MOG1-125 in complete Freund's adjuvant (CFA), followed by administration of pertussis toxin in PBS, first on the day of immunization and then again the following day. Typical EAE onset is 9 to 14 days after immunization, with peak of disease 3 to 5 days after onset for each mouse. The peak lasts 1 to 3 days, followed by partial recovery. Around 25% of mice will show an increase in EAE severity (relapse) after initial partial recovery. This usually occurs 20-27 days after immunization. Groups of 10 to 12 mice, with 4 to 6 mice per cage are used.

Engineered enucleated erythroid cells comprising MOG-MHCII-GPA and PD-L1 or MOG-MHCII-GPA and CD25-specific IL2 are formulated in a buffer appropriate for enucleated erythroid cells. Treatment with the engineered enucleated erythroid cells begins at the time of EAE onset. If the engineered enucleated erythroid cells are being tested for their ability to reverse the course of chronic EAE, treatment is initiated 7-14 days after disease onset. Mice are assigned to treatment groups as they develop EAE (rolling enrollment) or at a fixed time after immunization, but always in a balanced manner to achieve groups with similar time of EAE onset and similar EAE onset scores. If enrollment is after EAE onset, mice are also balanced for maximum score before enrollment.

Dosing occurs at a clinical score=1, as set out in Table 25, below.

Dosing of the animals may be carried out 1-3 times per day. The frequency of dosing is every 2-14 days, for example every 2, 3, 4, 5, 6, 7, 8, 9, 10, 11, 12, 13 or 14 days.

Assessment of the effects of engineered enucleated erythroid cells expressing MOG-MHCII-GPA and PD-L1 or erythroid cells comprising MOG-MHCII-GPA and CD25-specific IL2 are determined using one or more of (1) EAE scoring; (2) change in body weight and (3) histological analysis, as described in detail below.

EAE Scoring

Typically, EAE is scored on scale of 0 to 5, including “in-between” scores (i.e. 0.5, 1.5, 2.5, 3.5) when the clinical picture lies between two defined scores. The scoring method differs slightly depending on the stage of disease (onset/peak vs. recovery), for each individual mouse. To avoid unconscious bias in scoring, mice are scored blind, by a person unaware of which mice have received which treatment.

TABLE 25
Mouse EAE scoring - onset and peak

| Score | Clinical observations |
|---|---|
| 0.0 | No obvious changes in motor function compared to non-immunized mice. |
| | When picked up by base of tail, the tail has tension and is erect. Hind legs are usually |
| | spread apart. When the mouse is walking, there is no gait or head tilting. |
| 0.5 | Tip of tail is limp. |
| | When picked up by base of tail, the tail has tension except for the tip. Muscle straining |
| | is felt in the tail, while the tail continues to move. |
| 1.0 | Limp tail. |
| | When picked up by base of tail, instead of being erect, the whole tail drapes over |
| | finger. Hind legs are usually spread apart. No signs of tail movement are observed. |
| 1.5 | Limp tail and hind leg inhibition. |
| | When picked up by base of tail, the whole tail drapes over finger. When the mouse is |
| | dropped on a wire rack, at least one hind leg falls through consistently. Walking is very |
| | slightly wobbly. |
| 2.0 | Limp tail and weakness of hind legs. |
| | When picked up by base of tail, the legs are not spread apart, but held closer together. |
| | When the mouse is observed walking, it has a clearly apparent wobbly walk. One foot |
| | may have toes dragging, but the other leg has no apparent inhibitions of movement. |
| | -OR- |
| | Mouse appears to be at score 0.0, but there are obvious signs of head tilting when the |
| | walk is observed. The balance is poor. |
| 2.5 | Limp tail and dragging of hind legs. |
| | Both hind legs have some movement, but both are dragging at the feet (mouse trips on |
| | hind feet). |
| | -OR- |
| | No movement in one leg/completely dragging one leg, but movement in the other leg. |
| | -OR- |
| | EAE severity appears mild when picked up (as score 0.0-1.5), but there is a strong |
| | head tilt that causes the mouse to occasionally fall over. |
| 3.0 | Limp tail and complete paralysis of hind legs (most common). |
| | -OR- |
| | Limp tail and almost complete paralysis of hind legs. One or both hind legs are able to |
| | paddle, but neither hind leg is able to move forward of the hind hip. |
| | -OR- |
| | Limp tail with paralysis of one front and one hind leg. |
| | -OR- |
| | ALL of: |
| | Severe head tilting, |
| | Walking only along the edges of the cage, |
| | Pushing against the cage wall, |
| | Spinning when picked up by base of tail. |
| 3.5 | Limp tail and complete paralysis of hind legs. In addition to: |
| | Mouse is moving around the cage, but when placed on its side, is unable to right itself. |
| | Hind legs are together on one side of body. |
| | -OR- |
| | Mouse is moving around the cage, but the hind quarters are flat like a pancake, giving |
| | the appearance of a hump in the front quarters of the mouse. |
| 4.0 | Limp tail, complete hind leg and partial front leg paralysis. |
| | Mouse is minimally moving around the cage but appears alert and feeding. |
| | Often euthanasia is recommended after the mouse scores 4.0 for 2 days. However, with |
| | daily s.c. fluids most C57BL/6 mice may recover to 3.5 or 3.0, while SJL mice may |
| | fully recover even if they reach score 4.0 at the peak of disease. When the mouse is |
| | euthanized because of severe paralysis, a score of 5.0 is entered for that mouse for the |
| | rest of the experiment. |
| 4.5 | Complete hind and partial front leg paralysis, no movement around the cage. Mouse is |
| | not alert. |
| | Mouse has minimal movement in the front legs. The mouse barely responds to contact. |
| | Euthanasia is recommended. When the mouse is euthanized because of severe |
| | paralysis, a score of 5.0 is entered for that mouse for the rest of the experiment. |
| 5.0 | Mouse is spontaneously rolling in the cage (euthanasia is recommended). |
| | -OR- |
| | Mouse is found dead due to paralysis. |
| | -OR- |
| | Mouse is euthanized due to severe paralysis. |

In the recovery stage of EAE, most mice will have a tail that is no longer limp but is not normal either; it feels rigid and is “hooked”. The hind legs may start moving (pedaling), but the mouse cannot walk. Either change makes scoring difficult. Therefore, if this is the case, the following modifications to the above scoring criteria are used for these mice, shown below in Table 26:

TABLE 26
Mouse EAE scoring - modified

| Score | Clinical observations |
|---|---|
| 0.0 | When held by the base of tail, tail is somewhat “hooked” and rigid, but tail makes |
| | complete rotations around the body axis (“helicopter”). Mouse is healthy. No signs |
| | of wobbling. |
| 0.5 | Mouse appears normal but tail is “hooked” and rigid. Tail does not make complete |
| | rotations around the body axis (“helicopter”). Mouse is healthy. No signs of |
| | wobbling. |
| 3.0 | Mouse is found on its side (as described for score 3.5 above), but there is excessive |
| | hind leg movement. Mouse cannot walk. |
| | -OR- |
| | Mouse has a wobbly walk (as described for score 2.5 above), and is unable to take |
| | more than two steps without falling on its side. The mouse is unable to right itself. |
| | -OR- |
| | Mouse has poor movement in the hind legs (as described for score 2.5 above), and |
| | has partial front leg paralysis evidenced by head held lower than normal and |
| | mouse's inability to right itself when placed on its side. |
| All other | Subtract 0.5 from the score of all mice with either a rigid, “hooked” tail or pedaling |
| scores | of hind legs. |

Body Weight

During the course of EAE, changes in body weight reflect disease severity. Mice often lose a small amount of weight on the day following immunization. This appears to be due to effects of the administered adjuvant and pertussis toxin. Mice then steadily increase their body weight until disease onset. On the day of EAE onset, mice consistently lose 1-2 g of their body weight (5-10% of body weight). The weight loss continues with the progression of EAE severity, with the loss reaching around 20% of their pre-onset body weight at the peak of disease. The weight loss is most likely due to both paralysis and reduced food intake as well as high production of pro-inflammatory cytokines such as TNF during the acute phase of inflammation. After the peak of disease is reached, mice slowly gain weight, even if their clinical score does not improve. This increase in weight may be due to down regulation of inflammation which results in lower levels of pro-inflammatory cytokines in blood. Untreated or vehicle-treated mice usually have around 90% of their pre-immunization body weight 28 days after immunization.

Histology

Histological analysis is performed either at the end of the study (usually around 28 days after immunization) or at the time when the vehicle group reaches peak of disease (usually 14-18 days after immunization). Inflammation in EAE normally starts in the lumbar region of the spinal cord, spreading to the entire spinal cord by the peak of disease.

At onset of disease, the number of inflammatory foci correlates strongly with disease severity. The number of foci increases somewhat until the peak of disease, when 6-15 inflammatory foci/section are typically found throughout the spinal cord. In the chronic stage of EAE (starting several days after the peak of disease), many inflammatory foci resolve, typically resulting in 3-4 inflammatory foci in each spinal cord section by approximately 28 days after immunization.

Because the largest numbers of inflammatory foci are present early in the course of disease, if histological analysis is performed at the end of the study, mice which have late EAE onset often have more inflammatory foci in their spinal cords than might be expected from their clinical score. For example, in a 28 day study a mouse with EAE onset on 27 days after immunization and an end clinical score of 2 will likely have more inflammatory foci than a mouse with EAE onset 9 days after immunization and an end score of 3 5 Similarly, a mouse which relapses shortly before the end of the study (relapse is defined as 1 or more points of increase in clinical score) will usually have more inflammatory foci at the end of the study than a mouse with stable chronic disease, even if the two have the same clinical score at the end of the study.

Demyelination is usually not found during the first two days after disease onset, but is found at the peak of disease (4-5 days after EAE onset) and continues during the chronic phase of EAE. Demyelination scores do not change much between the peak and 28 days after immunization and usually average between 1.2 and 2.5.

Demyelination is scored in both Luxol fast blue stained sections (LFB) and in H&E sections. In LFB sections, spinal cord white matter stains dark blue and demyelinated areas are a lighter blue color, and are associated with large vacuoles. In H&E stained sections disruption of normal structure with large vacuoles is indicative of demyelination.

Apoptotic cells are identified in H&E sections, and are usually not found during the first two days of disease development. They are found at the peak and during the chronic stage of EAE. The average number of apoptotic cells is usually between 2 and 4 per section.

In embodiments, administration of the enucleated erythroid cells expressing MOG-MHCII-GPA and PD-L1 or erythroid cells expressing MOG-MHCII-GPA and CD25-specific IL2 will lead to one or more of an improvement in EAE scoring or a reduction in the number of inflammatory foci compared to vehicle treated control mice.

### Example 5. Generation of Erythroid Cells Genetically Engineered to Express a PR1-HLA-A2-GPA Fusion Protein

### Results

Erythroid cells are transduced to express a fusion protein comprising PR1, a human leukocyte antigen (HLA)-A2 restricted peptide, fused to an exogenous antigen presenting polypeptide, MHCI HLA-A2, fused to the GPA transmembrane domain (GPA) (PR1-HLA-A2-GPA). FIG. 1B shows a schematic of the design for expressing PR1 peptide and MHCI as a single chain fusion. Cell culture and transduction is performed as described in the “Methods” section below to yield erythroid cells expressing PR1 presented by MHCI on the surface, anchored with a GPA transmembrane domain.

Binding of an APC-labelled or PE-labelled anti-PR1 antibody is used to validate expression of the antigenic peptide in the engineered erythroid cells. Binding of an APC-labelled or PE-labelled anti-MHCI antibody is used to validate expression of the MHCI antigen presenting peptide in the engineered erythroid cells.

### Methods

Production of Lentiviral Vector

The gene encoding the PR1-HLA-A2-GPA fusion protein is cloned into the multiple cloning site of lentivirus vector pCDH with the MSCV promoter sequence from System Biosciences. Lentivirus is produced in 293T cells by transfecting the cells with pPACKH1 (System Biosciences) and pCDH lentivirus vector containing PR1-HLA-A2-GPA gene. Cells are then placed in fresh culturing medium. The virus supernatant is collected 48 hours post-medium change by centrifugation at 1,500 rpm for 5 minutes. The supernatant is collected and frozen in aliquots at −80° C.

Expansion and Differentiation of Erythroid Cells

Human CD34+ cells derived from mobilized peripheral blood cells from normal human donors are purchased frozen from AllCells Inc. The expansion/differentiation procedure comprises 3 stages. In the first stage, thawed CD34+ erythroid precursors are cultured in Iscove's MDM medium comprising recombinant human insulin, human transferrin, recombinant human recombinant human stem cell factor, and recombinant human interleukin 3. In the second stage, erythroid cells are cultured in Iscove's MDM medium supplemented with bovine serum albumin, recombinant human insulin, human transferrin, human recombinant stem cell factor, human recombinant erythropoietin, and L-glutamine. In the third stage, erythroid cells are cultured in Iscove's MDM medium supplemented with human transferrin, recombinant human insulin, human recombinant erythropoietin, and heparin. The cultures are maintained at 37° C. in 5% CO2 incubator.

Transduction of Erythroid Precursor Cells

Erythroid precursor cells are transduced during step 1 of the culture process described above. Erythroid cells in culturing medium are combined with lentiviral supernatant and polybrene. Infection is achieved by spinoculation, spinning the plate at 2000 rpm for 90 minutes at room temperature. After spinoculation, the cells are incubated at 37° C. overnight.

Antibody Binding

Binding of an allophycocyanin (APC)-labelled or Phycoerythrin (PE)-labelled anti-PR1-HLA-A2 antibody (e.g. anti-PR1/HLA-A2 monoclonal antibody Hu8F4) is used to validate expression of PR1-HLA-A2-GPA fusion protein in the engineered erythroid cells. Binding of the antibody is measured by flow cytometry for APC fluorescence or PE fluorescence. A gate is set based on stained untransduced cells.

Binding of an APC-labelled or PE-labelled anti-MHCI antibody (e.g. HLA-A2 Antibody (MA1-80117), Invitrogen)) is used to validate expression of the MHCI antigen presenting peptide in the engineered erythroid cells. Binding of any one of the antibodies is measured by flow cytometry for APC fluorescence or PE fluorescence. A gate is set based on stained untransduced cells.

### Example 6. Generation and In Vitro Validation of Erythroid Cells Genetically Engineered to Co-Express a PR1-HLA-A2-GPA Fusion Protein and a Costimulatory Polypeptide

### Results

Erythroid cells are transduced to express a fusion protein comprising PR1, a human leukocyte antigen (HLA)-A2 restricted peptide, fused to an exogenous antigen presenting polypeptide, MHCI HLA-A2, fused to the GPA transmembrane domain (PR1-HLA-A2-GPA). FIG. 1B shows a schematic of the design for expressing PR1 peptide and MHCI as a single chain fusion. The erythroid cells are also transduced to additionally express an exogenous costimulatory peptide, 4-1BBL. Cell culture and transduction is performed as described in “Methods” section below to yield erythroid cells expressing PR1 presented by MHCI on the surface, anchored with a GPA transmembrane domain.

Binding of an APC-labelled or PE-labelled anti-PR1 or anti-4-1BBL antibody to the exogenous peptides PR1 and 4-1BBL is used to validate expression of the respective peptides in the engineered erythroid cells. Binding of an APC-labelled or PE-labelled anti-MHCI antibody is used to validate expression of the MHCI antigen presenting peptide in the engineered erythroid cells.

Functional activity of the effect of the engineered erythroid cells (PR1-HLA-A2-GPA, 4-1BBL) is assessed for their ability to stimulate the initial activation and proliferation of primary CD8+ T cells.

### Methods

Production of Lentiviral Vector

The genes for PR1-HLA-A2-GPA fusion protein and 4-1BBL are constructed. Genes encoding the proteins are cloned into the multiple cloning site of lentivirus vector pCDH with the MSCV promoter sequence from System Biosciences, such that one vector comprises the genes for both exogenous proteins. Lentivirus is produced in 293T cells by transfecting the cells with pPACKH1 (System Biosciences) and pCDH lentivirus vector containing genes for PR1-HLA-A2-GPA and 4-1BBL. Cells are placed in fresh culturing medium. The virus supernatant is collected 48 hours post-medium change by centrifugation at 1,500 rpm for 5 minutes. The supernatant is collected and frozen in aliquots at −80° C.

Transduction of Erythroid Precursor Cells

Expansion and differentiation of erythroid cells is performed according to Example 5, with the lentiviral vectors described above. Erythroid precursor cells are transduced during step 1 of the culture process. Erythroid cells in culturing medium are combined with lentiviral supernatant and polybrene. Infection is achieved by spinoculation, spinning the plate at 2000 rpm for 90 minutes at room temperature. After spinoculation, the cells are incubated at 37° C. overnight

Antibody Binding

Binding of an APC-labelled or PE-labelled anti-PR1 antibody is used to validate expression of the PR1-HLA-A2-GPA fusion in the engineered erythroid cells, as described in Example 5. Binding of an APC-labelled or PE-labelled anti-MHCI antibody is used to validate expression of the MHCI antigen presenting polypeptide in the engineered erythroid cells, also as described in Example 5. Binding of an APC-labelled or PE-labelled anti-4-1BBL antibody (R&D Systems) is used to validate expression of 4-1BBL in in the engineered erythroid cells. Binding of any one of the antibodies is measured by flow cytometry for APC fluorescence or PE fluorescence. A gate is set based on stained untransduced cells.

Functional Validation Assays

The erythroid cells genetically engineered to co-express a PR1-HLA-A2-GPA fusion protein and a 4-1BBL costimulatory polypeptide are tested for their ability to stimulate the initial activation and proliferation of primary CD8+ T cells. The T cells are stimulated with the engineered erythroid cells, and the following parameters are evaluated: (1) rate of growth of the T cells stimulated with the aAPCs, as determined by a standard thymidine incorporation assay, which gives the total amount of DNA synthesized in a bulk culture; (2) induction of proliferation and cell division of CD4+ T cells; (3) viability of the CD8+ T cells stimulated by the various aAPCs during culture by fluorescent staining with annexin V and propidium iodide; (4) Bcl-xL and IL-2 expression, two genes involved in T cell survival and proliferation, respectively, using quantitative real time RT-PCR in order to determine the levels of steady-state mRNA coding for Bcl-xL and IL-2.

Cytokine Production

Cytokines are important effector molecules and provide insight into T cell differentiation. The ability of erythroid cells engineered to co-express a PR1-HLA-A2-GPA fusion and the costimulatory peptide 4-1BBL to induce certain cytokines from CD8 T cells is quantitated, for example IL-2 (a key T cell growth factor for ex vivo expansion and a cell's ability to induce IL-2 correlates well with its long term growth potential); IL-4 (a marker for TH2 differentiation); and IL-10 (an immunosuppressive cytokine that may be surrogate for T regulatory cell outgrowth). Other cytokines include, but are not limited to, TGF-β (for the same rationale as IL-10); IFN-γ (a marker for TH1 differentiation and an important effector cytokine); and TNFα (an important effector cytokine). Quantitation can be carried out using the ELISPOT (enzyme-linked immunospot) technique, that detects T cells that secrete a given cytokine (e.g., gamma interferon [IFN-γ]) in response to an antigenic stimulation. T cells are cultured with antigen-presenting cells in wells which have been coated with anti-IFN-γ antibodies. The secreted IFN-γ is captured by the coated antibody and then revealed with a second antibody coupled to a chromogenic substrate. Thus, locally secreted cytokine molecules form spots, with each spot corresponding to one IFN-γ-secreting cell. The number of spots allows one to determine the frequency of IFN-γ-secreting cells specific for a given antigen in the analyzed sample. The ELISPOT assay has also been described for the detection of tumor necrosis factor alpha, interleukin-4 (IL-4), IL-5, IL-6, IL-10, IL-12, granulocyte-macrophage colony-stimulating factor.

### Example 7. Activity of Enucleated Erythroid Cells Engineered to Co-Express a PR1-HLA-A2-GPA Fusion Protein and a Costimulatory Polypeptide in an Acute Myelogenous Leukemia (AML) Murine Cancer Model In Vivo

PR1 peptide has been shown to be a human leukemia antigen. PR1-specific cytotoxic T lymphocytes (CTL) elicited in vitro from healthy donors have been shown to lyse P3-expressing AML cells from patients. Further, it has been shown that adoptive transfer of PR1-CTL generated in vitro is associated with reduced AML cells in NOD/SCID mice (Molldrem et al., Cancer Res. 1999; 59:2675-2681; Molldrem et al., Nat Med. 2000; 6:1018-1023; Rezvani et al., Blood. 2003; 102:2892-2900; the entire contents of each of which are incorporated by reference herein).

The well-established NOD/SCID mouse is used as a model to determine whether bone marrow aspirate from mice that received PR1-CTL have more AML blasts in a hypercellular marrow, compared to mice receiving PR1-CTL plus the enucleated erythroid cells engineered to co-express a PR1-HLA-A2-GPA fusion protein and 4-1BBL. The model is also used to determine if PR1-CTL levels are higher in mice receiving enucleated erythroid cells engineered to co-express a PR1-HLA-A2-GPA fusion protein and 4-1BBL as compared to mice that did not receive the enucleated erythroid cells. The model is also used to determine if mice receiving enucleated erythroid cells engineered to co-express a PR1-HLA-A2-GPA fusion protein and 4-1BBL maintain a CD45RA-CD28+ effector phenotype as compared to mice that did not receive the enucleated erythroid cells.

PR1-CTL Production

PR1-CTL are expanded in bulk culture using a method described previously, with some modification (Molldrem et al., Blood. 1996; 88:2450-2457, the entire content of which is incorporated by reference herein). Autologous dendritic cells (DC) are generated from a HLA-A2.1+ healthy donor. Briefly, adherent monocytes from a normal donor are stimulated for 7 days with a combination of cytokines granulocyte-macrophage colony-stimulating factor (GM-CSF) (500 IU/mL) and interferon (INF)-α. The activated DC are collected, and a fraction of the cells is analyzed by FACS for CD80 and CD14 expression. In the presence of IL-2 (20 IU/m1), DC are pulsed with PR1, PR2 or pp65 peptides. PR2 (RLFPDFFTRV (SEQ ID NO: 720)) is another HLA-A2-restricted peptide derived from proteinase 3, but PR2-CTL are incapable of killing leukemia cells (Molldrem et al., Blood. 1996). Peripheral blood mononuclear cells (PMBC) are stimulated weekly with peptide-pulsed autologous DC for 3-4 weeks. A fraction of PR1-CTL is harvested and tested for specific lysis using a CTL cytotoxicity assay used previously (Molldrem et al., Blood. 1996, the entire content of which is incorporated by reference herein). The remaining CTL from bulk culture is purified with a sorter using antibodies to deplete CD4, B and natural killer (NK) cells simultaneously.

NOD/SCID AML Xenograft Model

The NOD/SCID-HLA-A2.1 mice are used for the engraftment of AML cells. Human AML bone marrow samples are obtained, and AML cells from patients are transferred intravenously into irradiated (200 cGy) NOD/SCID mice. AML bone marrow samples are chosen based on the ability to engraft successfully in bone marrow at various doses 2 weeks post-transfer. A small number of PR1-specific T cells are adoptively transferred to the NOD-SCID mouse engrafted with AML, either alone or together with enucleated erythroid cells engineered to co-express a PR1-HLA-A2-GPA fusion protein and 4-1BBL. Animals are killed 2 weeks post-transfer, and tissues are harvested for flow cytometry (FACS) and immunohistochemistry (IHC) analysis. Human CD45 Ab and mouse CD45.2 Ab are used to identify cells of human origin. The differential cell counts are performed on cyto spin preparations and smears prepared from bone marrow. Formalin-fixed paraffin-embedded sections of tissue organs harvested are stained with hematoxylin and eosin stain.

CD45RA-CD28+ Effector Phenotype

An APC-labelled or PE-labelled anti-CD45RA antibody (Biolegend) and an APC-labelled or PE-labelled anti-CD28 antibody (Biolegend) is used to validate CD45RA-CD28+ effector phenotype. Binding of the antibody is measured by flow cytometry for APC fluorescence or PE fluorescence. A gate is set based on stained untransduced cells.

In embodiments, bone marrow aspirate from mice that received PR1-CTL have more AML blasts in a hypercellular marrow, compared to mice receiving PR1-CTL plus the enucleated erythroid cells engineered to co-express a PR1-HLA-A2-GPA fusion protein and 4-1BBL. In other embodiments, PR1-CTL levels are higher in mice receiving enucleated erythroid cells engineered to co-express a PR1-HLA-A2-GPA fusion protein and 4-1BBL as compared to mice that did not receive the enucleated erythroid cells. In other embodiments, mice receiving enucleated erythroid cells engineered to co-express a PR1-HLA-A2-GPA fusion protein and 4-1BBL maintain a CD45RA-CD28+ effector phenotype as compared to mice that did not receive the enucleated erythroid cells.

### Example 8. Generation and In Vitro Validation of Erythroid Cells Genetically Engineered to Co-Express a Gp100-H-2Db-GPA Fusion Protein and a Costimulatory Polypeptide

### Results

Erythroid cells are transduced to express a fusion protein comprising gp100, a melanoma antigen, fused to an exogenous antigen presenting polypeptide, MHCI H-2Db, fused to the GPA transmembrane domain (gp100-H-2Db-GPA). The erythroid cells are also transduced to additionally express an exogenous costimulatory peptide, 4-1BBL. Cell culture and transduction is performed as described in the “Methods” section below to yield erythroid cells expressing gp100 presented by MHCI on the surface, anchored with a GPA transmembrane domain.

Binding of an APC-labelled or PE-labelled anti-gp100 or anti-41-BBL antibody to the exogenous peptides gp100 and 4-1BBL is used to validate expression of the respective peptides in the engineered erythroid cells. Binding of an APC-labelled or PE-labelled anti-MHCI antibody is used to validate expression of the MHCI antigen presenting peptide in the engineered erythroid cells.

Functional activity of the effect of the engineered erythroid cells (gp100-H-2Db-GPA, 4-1BBL) is assessed for their ability to stimulate the initial activation and proliferation of primary CD8+ T cells.

Methods

Production of Lentiviral Vector

The genes for gp100-H-2Db-GPA fusion protein and 4-1BBL are constructed. Genes encoding the proteins are cloned into the multiple cloning site of lentivirus vector pCDH with the MSCV promoter sequence from System Biosciences, such that one vector comprises the genes for both exogenous proteins. Lentivirus is produced in 293T cells by transfecting the cells with pPACKH1 (System Biosciences) and pCDH lentivirus vector containing genes for gp100-H-2Db and 4-1BBL. Cells are placed in fresh culturing medium. The virus supernatant is collected 48 hours post-medium change by centrifugation at 1,500 rpm for 5 minutes. The supernatant is collected and frozen in aliquots at −80° C.

Transduction of Erythroid Precursor Cells

Expansion and differentiation of erythroid cells is performed according to Example 5, with the lentiviral vector described above. Erythroid precursor cells are transduced during step 1 of the culture process. Erythroid cells in culturing medium are combined with lentiviral supernatant and polybrene. Infection is achieved by spinoculation, spinning the plate at 2000 rpm for 90 minutes at room temperature. After spinoculation, the cells are incubated at 37° C. overnight.

Antibody Binding

Binding of biotinylated anti-gp100 antibody (e.g. TB-M505-M, MBL International Corp.) is used to validate expression of gp100-H-2Db-GPA fusion protein in the engineered erythroid cells. Binding of the biotinylated antibody is detected with avidin conjugates in cell surface labeling and flow cytometry/fluorescence-activated cell sorting (FACS). Binding of an APC-labelled or PE-labelled anti-MHCI antibody (e.g. MHC Class I (H-2Db) Monoclonal Antibody (28-14-8), PE, Thermofisher Scientific) is used to validate expression of the MHCI antigen presenting peptide in the engineered erythroid cells. Binding of an APC-labelled or PE-labelled anti-4-1BBL antibody (R&D Systems) is used to validate expression of 4-1BBL in in the engineered erythroid cells. Binding of any one of the antibodies is measured by flow cytometry for APC fluorescence or PE fluorescence. A gate is set based on stained untransduced cells.

Functional Validation Assays

The erythroid cells genetically engineered to co-express a gp100-H-2Db-GPA fusion protein and a 4-1BBL costimulatory polypeptide are tested for their ability to stimulate the initial activation and proliferation of primary CD8+ T cells. The T cells are stimulated with the engineered erythroid cells, and the following parameters are evaluated: (1) rate of growth of the T cells stimulated with the aAPCs, as determined by a standard thymidine incorporation assay, which gives the total amount of DNA synthesized in a bulk culture; (2) induction of proliferation and cell division of CD4+ T cells; (3) viability of the CD8+ T cells stimulated by the various aAPCs during culture by fluorescent staining with annexin V and propidium iodide; (4) Bcl-xL and IL-2 expression, two genes involved in T cell survival and proliferation, respectively, using quantitative real time RT-PCR in order to determine the levels of steady-state mRNA coding for Bcl-xL and IL-2.

Cytokine Production

The ability of erythroid cells engineered to co-express a gp100-H-2Db-GPA fusion and the costimulatory peptide 4-1BBL to induce certain cytokines from CD8 T cells is quantitated, for example, IL-2 (a key T cell growth factor for ex vivo expansion and a cell's ability to induce IL-2 correlates well with its long term growth potential); IL-4 (a marker for TH2 differentiation); and IL-10 (an immunosuppressive cytokine that may be surrogate for T regulatory cell outgrowth). Other cytokines include, but are not limited to, TGF-β (for the same rationale as IL-10); IFN-γ (a marker for TH1 differentiation and an important effector cytokine); and TNFα (an important effector cytokine). Quantitation can be carried out using the ELISPOT (enzyme-linked immunospot) technique, that detects T cells that secrete a given cytokine (e.g., gamma interferon [IFN-γ]) in response to an antigenic stimulation. T cells are cultured with antigen-presenting cells in wells which have been coated with anti-IFN-γ antibodies. The secreted IFN-γ is captured by the coated antibody and then revealed with a second antibody coupled to a chromogenic substrate. Thus, locally secreted cytokine molecules form spots, with each spot corresponding to one IFN-γ-secreting cell. The number of spots allows one to determine the frequency of IFN-γ-secreting cells specific for a given antigen in the analyzed sample. The ELISPOT assay has also been described for the detection of tumor necrosis factor alpha, interleukin-4 (IL-4), IL-5, IL-6, IL-10, IL-12, granulocyte-macrophage colony-stimulating factor.

### Example 9. Activity of Enucleated Erythroid Cells Engineered to Co-Express a Gp100-H-2Db-GPA Fusion Protein and a Costimulatory Polypeptide in a B-16 Mouse Tumor Model for Melanoma

A subset of patients with metastatic melanoma can be successfully treated by the administration of recombinant interleukin-2 (rIL-2), sometimes given together with autologous melanoma-reactive lymphocytes that have been expanded ex vivo (Rosenberg, 1997; Rosenberg, 1999). Recently, a number of different laboratories have used these anti-tumor lymphocytes to clone melanoma-associated antigens, which have generally been nonmutated melanocyte differentiation antigens (MDA), a group that includes gp100 (Overwijk and Restifo, Curr Protoc Immunol. 2001 May; CHAPTER: Unit-20.1, the entire content of which is incorporated herein by reference).

The subcutaneous model is widely used for the evaluation of therapy in many tumor models, including B16 melanoma. Upon subcutaneous injection, B16 will form a palpable tumor in 5 to 10 days and grow to a 1×1×1-cm tumor in 14 to 21 days. When allowed to grow larger, the tumors often become necrotic in the center and begin to ulcerate or bleed; it is advisable to sacrifice the mice before this point. The typical dose used is 1×10⁵ cells/mouse, which is 1.5 to 2 times the minimal tumorigenic dose in normal C57BL/6 mice.

The B16 melanoma mouse model is used as a model to determine whether enucleated erythroid cells engineered to co-express a gp100-H-2Db-GPA fusion and the costimulatory peptide 4-1BBL, can eliminate B16 melanoma cells in the mouse.

Methods for B-16 cell preparation and inoculation of mice are described in Overwijk and Restifo (Curr Protoc Immunol. 2001 May; CHAPTER: Unit-20.1, incorporated by reference in its entirety herein). Dosing with the engineered erythroid cells is started when the tumors become palpable. The cells are formulated in PBS or other buffer.

In embodiments, administration of the engineered enucleated erythroid cells will lead to one or both of inhibition of tumor cell engraftment or reduction of the tumor size, compared to unmodified control cells.

### Example 10. Erythroid Cells Engineered to Present MHCI (Ovalbumin) and 4-1BBL Activate Ova-Specific T Cells In Vitro

Murine erythroid cells were conjugated with MHCI presenting ovalbumin peptide and 4-1BBL using the click methodology (click chemistry for functionalizing erythroid cells is described in International Application No. PCT/US2018/000042, which claims priority to U.S. Provisional Application No. 62/460,589, filed Feb. 17, 2017 and U.S. Provisional Application No. 62/542,142, filed Jul. 8, 2017, incorporated by reference in their entireties herein). Briefly, mouse peripheral blood was filtered through a PAL de-leukocyte filter and labeled with 0.04 mM 6′ Azido-NHS-ester in pH8 PBS for 30 min at room temperature. Azido-labeled mRCT were incubated with 40 uM m4-1BBL-DBCO-Thiolinker at room temperature for 1h and further incubated at 4 C overnight. mRCT-m4-1BBL were clicked with biotin by incubating with 1 uM water soluble DBCO biotin at room temperature for 1h, followed by incubating with neutravidin at a 1:1 molar ratio of biotin and neutravidin. Neutravidin bound cells were then incubated with biotinylated H-2Kb SIINFEKL (SEQ ID NO: 721) monomer at a 1:1 molar ratio of biotin and monomer for 1h at room temperature.

CD8+ T cells were purified from secondary lymphoid organs of OT1 transgenic mice using a negative selection kit from Miltenyi and labeled with 1 uM CFSE. 2E5 CFSE labeled OT1 CD8+ T cells were plated into each well of a 96 well plate. Varying amounts (3E6, 1E6, 3.3E5, or 1.1E5 cells) of mRCT, mRCT-4-1BBL, mRCT-MHC(ova), or mRCT-MHC(ova)+4-1BBL were plated in cRPMI media into the wells containing OT1 CD8+ T cells and incubated for two days at 37 C. Culture supernatant was collected to measure IL2 and IFN-γ concentration by cytokine ELISA. Cells were washed and stained with anti-CD8, livedead dye, and anti-CD44 to quantify T cell expansion and activation.

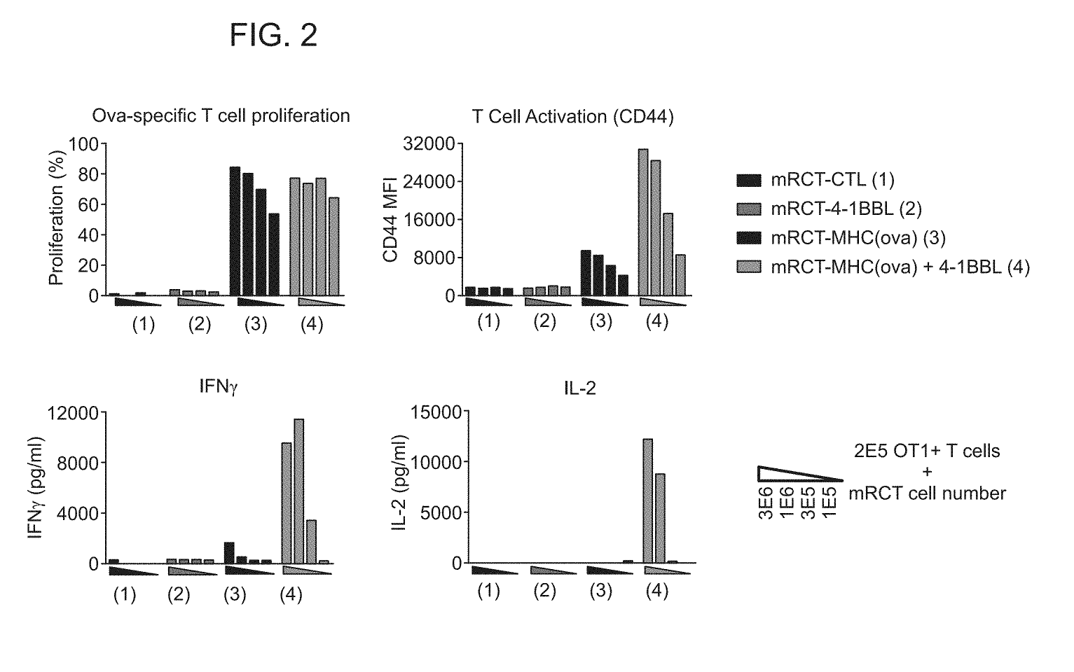

As shown in FIG. 2, murine erythroid cells presenting MHC I (ovalbumin) and 4-1BBL on the cell surface, or murine erythroid cells presenting MHC I (ovalbumin) alone (RCT-aAPC (ova)), potently activate ova-specific T cells in vitro. These results demonstrate the ability to potently and selectively expand and activate an antigen-specific T cell using the engineered enucleated cells as described herein.

In similar studies to those presented above, human erythroid cells were transduced to co-express ova and 4-1BBL, rather than conjugated using the click methodology. In these experiments, CD34+ cells from a healthy human donor were expanded and transduced with HA-GPA, GPA-m4-1BBL, or b2ML-OVAH2Kb-GPA concentrated Lentivirus at MOI 20. hRCT-MHC(ova)+4-1BBL were generated by double (simultaneous) transduction with GPA-m4-1BBL and b2ML-OVAH2Kb-GPA concentrated Lentivirus at MOI 20 respectively. hRCT were harvested at maturation day 9 and filtered through PAL de-leukocyte filter to enrich for enucleated cells. Expression level and copy number were determined by staining with anti-m4-1BBL and anti-SIINFEKL (SEQ ID NO: 721) bound H2Kb and calibrated according to Bangs beads standard curves.

CD8+ T cells were purified from secondary lymphoid organs of OT1 transgenic mice using negative selection kit from Miltenyi and labeled with 1 uM CFSE. 3E5 CFSE labeled OT1 CD8+ T cells are plated into each well of a 96 well plate. Varying amounts (3E6, 6E5, 3E5 or 1.5E5 cells) of hRCT-HA-GPA, hRCT-m4-1BBL, hRCT-MHC(ova), or hRCT-MHC(ova)+4-1BBL were plated into the wells containing OT1 CD8+ T cells. The number of hRCT-m4-1BBL and hRCT-MHC(ova) cells in each well were adjusted so that the number of molecules of m4-1BBL and MHC(ova) match the total molecule numbers in wells of the corresponding hRCT-MHC(ova)+m4-1BBL. hRCT and OT1 cells were incubated at 37 C for 3 days. Culture supernatant was collected to measure IL2 and IFN-γ concentration by cytokine ELISA. The cells were washed and stained with anti-CD8, livedead dye, and anti-CD44 to quantify T cell expansion and activation. The results from this study showed that human erythroid cells transduced to co-express ova and m4-1BBL potently activate the ova-specific T cells in vitro demonstrating high CD44 expression, similar to the results obtained using mRCTs constructed with the click technology (described above and presented in FIG. 2).

### Example 11. Ovalbumin-Specific T Cells Expanded and Activated by Erythroid Cells Presenting MHCI (Ovalbumin) and 4-1BBL Selectively Kill Ovalbumin-Expressing Tumor Cells In Vitro

Murine erythroid cells were conjugated with MHCI presenting ovalbumin peptide and 4-1BBL using the click methodology (click chemistry for functionalizing erythroid cells is described in U.S. Provisional Application No. 62/460,589, filed Feb. 17, 2017 and U.S. Provisional Application No. 62/542,142, filed Jul. 8, 2017, incorporated by reference in their entireties herein). Briefly, mouse peripheral blood was filtered through a PAL de-leukocyte filter and labeled with 0.04 mM 6′ Azido-NHS-ester in pH8 PBS for 30 min at room temperature. Azido-labeled mRCT were incubated with 40 uM m4-1BBL-DBCO-Thiolinker at room temperature for 1h and further incubated at 4 C overnight. mRCT-m4-1BBL were clicked with biotin by incubating with 1 uM water soluble DBCO biotin at room temperature for 1h, followed by incubating with neutravidin at a 1:1 molar ratio of biotin and neutravidin. Neutravidin bound cells were then incubated with biotinylated H-2Kb SIINFEKL (SEQ ID NO: 721) monomer at a 1:1 molar ratio of biotin and monomer for 1h at room temperature.

CD8+ T cells were purified from secondary lymphoid organs of OT1 transgenic mice using negative selection kit from Miltenyi and labeled with 1 uM CFSE. 1.2E6 CFSE labeled OT1 CD8+ T cells were cultured with 1.2E7 aAPC (mRCT-4-1BBL H2Kb OVA) in each well of a 24 well plate. OT1 cells were harvested after a three day incubation and treated with ACK buffer to lyse the mRCT. To test the activity of the OT1 cells on tumor cells, 1E4 cell trace far red-labeled tumor cells, either parental tumor cells, EL4, or tumor cells expressing ovalbumin, EG7.OVA, (each target cells) were then plated into each well of 96 well U bottom plates. OT1 cells (effector cells) were added into the wells at 10:1, 5:1, 2:1, 1:1 or 0:1 effector to target (E:T) ratio. After a 22 hour incubation, cells were stained with livedead dye and fixed with 2% paraformaldehyde to enumerate live target cells in each well.

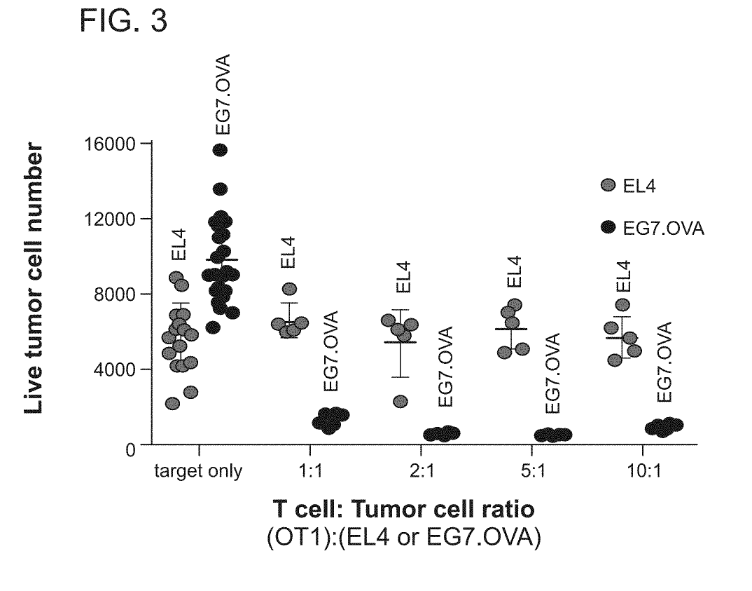

The results of this experiment are shown in FIG. 3. Strikingly, ovalbumin-specific T cells (OT1-T cells) that were expanded and activated by murine erythroid cells presenting MHC I (ovalbumin) and 4-1BBL were observed to selectively kill ovalbumin-expressing tumor cells, or EG7.OVA cells, while the parental cells that do not express ovalbumin (EL4 cells) were not attacked and killed. The ability to significantly expand and activate a specific tumor specific T cell population to kill tumors in vivo has parallels with CAR T approaches, which administer a tumor specific T cell population that can expand, sometimes uncontrollably, in the patient. A significant advantage of the present invention is that, by controlling the dose of the engineered enucleated cells described herein, the expansion of the tumor-specific T cells and ultimately the safety and efficacy of therapy can be more effectively controlled.

### Example 12. Erythroid Cells Engineered to Present MHCI (Ovalbumin) and 4-1BBL Activate Ova-Specific T Cells, which Traffic to Lymph Nodes, In Vivo

Murine erythroid cells were conjugated with MHCI presenting ovalbumin peptide and 4-1BBL using the click methodology (click chemistry for functionalizing erythroid cells is described in U.S. Provisional Application No. 62/460,589, filed Feb. 17, 2017 and U.S. Provisional Application No. 62/542,142, filed Jul. 8, 2017, incorporated by reference in their entireties herein). Briefly, mouse peripheral blood was filtered through a PAL de-leukocyte filter and labeled with 0.04 mM 6′ Azido-NHS-ester in pH8 PBS for 30 min at room temperature. Azido-labeled mRCT were incubated with 40 uM m4-1BBL-DBCO-Thiolinker at room temperature for 1h and further incubated at 4 C overnight. mRCT-m4-1BBL were clicked with biotin by incubating with 1 uM water soluble DBCO biotin at room temperature for 1h, followed by incubating with neutravidin at a 1:1 molar ratio of biotin and neutravidin. Neutravidin bound cells were then incubated with biotinylated H-2Kb SIINFEKL (SEQ ID NO: 721) monomer at a 1:1 molar ratio of biotin and monomer for 1h at room temperature.

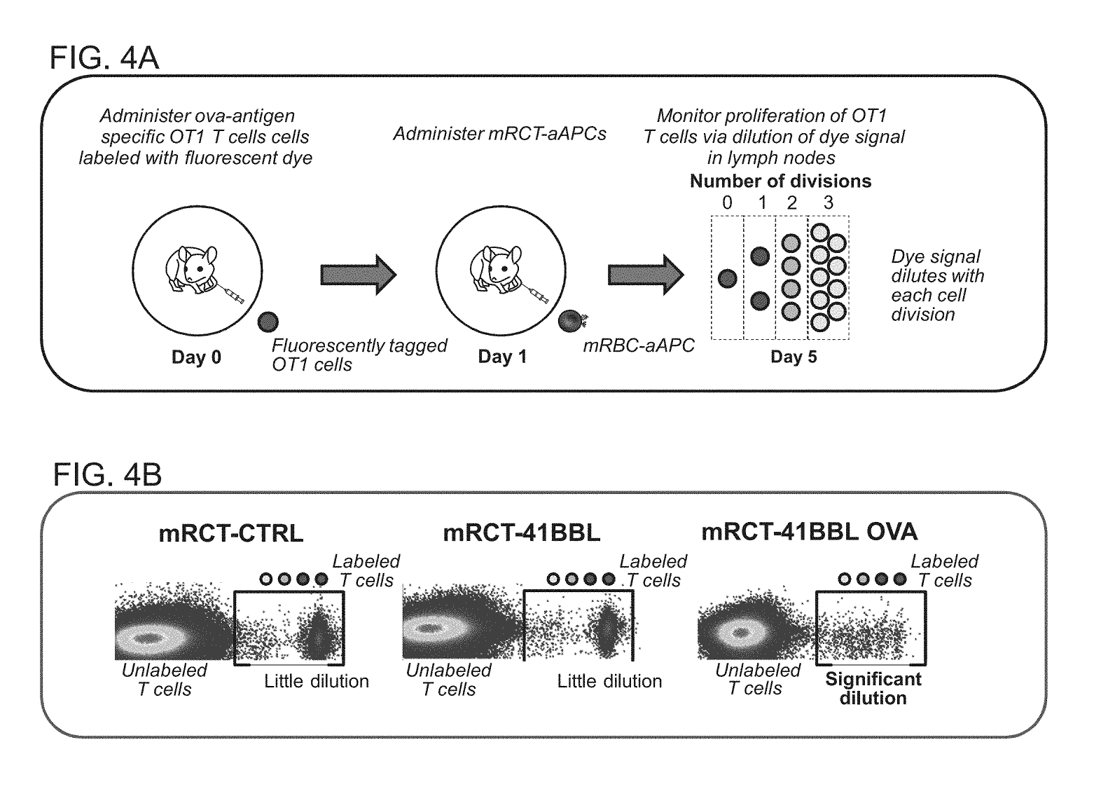

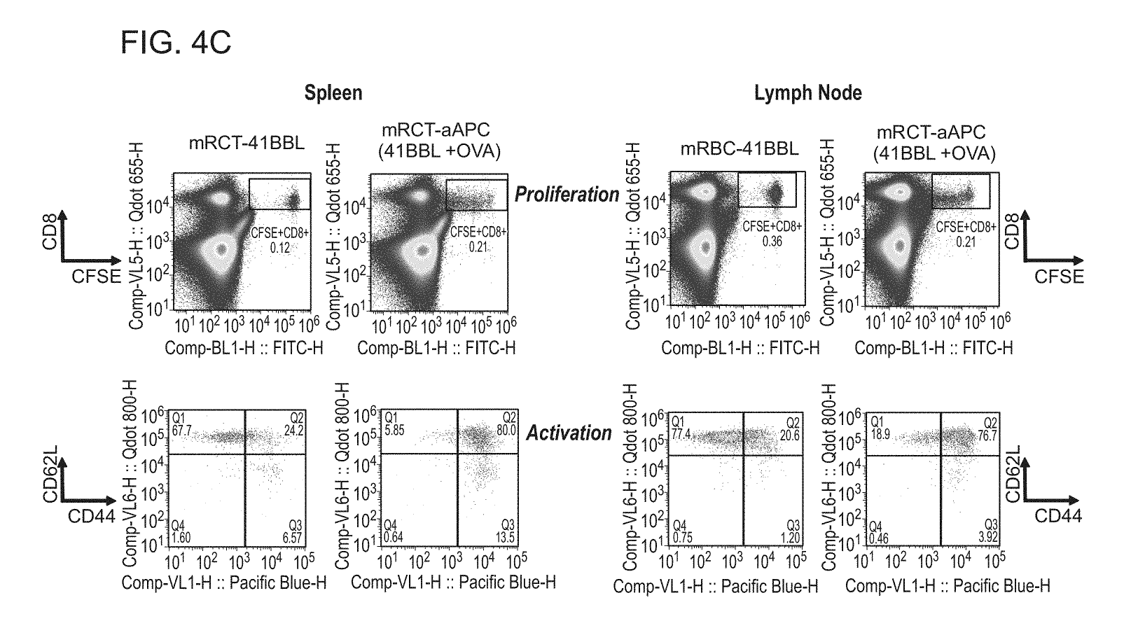

CD8+ T cells were purified from secondary lymphoid organs of RAG2−/−OT1 transgenic mice using negative selection kit from Miltenyi and labeled with fluorescent dye (10 uM CFSE). 1.8E6 OT1 cells were injected intravenously into C57BL/6 mice on day −1. On day 0, mice were injected intravenously with 1E8 aAPC (mRCT-4-1BBL or mRCT-4-1BBL H2Kb OVA). On day 4, mice were sacrificed and single cell suspensions from their spleen and lymph nodes were stained with NIR Zombie, APC 41BB, Pacific blue CD44, BV650 CD8 and BV785 CD62L and analyzed by flow cytometry. Proliferation of OT1 T cells was monitored by following dilution of fluorescent dye signal in lymph nodes or spleen. A schematic depicting the design of the experiment is shown in FIG. 4A. The results of the experiment are shown in FIGS. 4B and 4C, where FIG. 4B is a schematic of representative data, showing that mRCT-4-1BBL OVA specifically expand and activate OT1-T cells, while mRCT-4-1BBL without MHCI presenting ovalbumin peptide on the cell surface do not expand and activate OT1-T cells.

The results of this experiment in vivo in mice, as shown in FIG. 4C, demonstrated that mRCT-4-1BBL H2Kb OVA specifically expand and activate OTI-T cells as evidenced by increased CD44 expression. Further, it was found that the majority of OT1-T cells being activated display a central memory phenotype, which has been found to be a key population driving the effectiveness of T cell based therapies. The results further showed that these OTI-T cells traffic to the lymph nodes, where they are expected to undergo further expansion and activation, supporting their potential to effectively mobilize within the body and to the tumor to support a robust anti-tumor response In contrast, mRCT-4-1BBL without MHCI presenting ovalbumin peptide on the cell surface did not expand or activate OTI-T cells, thereby indicating that mRCT-aAPCs mimic the function of antigen presenting cells in vivo.

### Example 13. Generation and In Vitro Validation of Erythroid Cells Genetically Engineered to Co-Express a Gp350-HLA-A2-GPA Fusion Protein and a Costimulatory Polypeptide

### Results

Erythroid cells are transduced to express a fusion protein comprising an immunogenic peptide from an Epstein Barr virus-encoded glycoprotein 350 (gp350) that is (HLA)-A2 restricted, fused to an exogenous antigen presenting polypeptide, MHCI HLA-A2, fused to the GPA transmembrane domain (gp350-HLA-A2-GPA). An exemplary gp350 immunogenic peptide used is VLQWASLAV (SEQ ID NO: 698). The erythroid cells are also transduced to additionally express an exogenous costimulatory peptide, 4-1BBL. Cell culture and transduction is performed as described in “Methods” section below to yield erythroid cells expressing gp350 presented by MHCI on the surface, anchored with a GPA transmembrane domain.

Binding of an APC-labelled or PE-labelled anti-gp350 or anti-4-1BBL antibody to the exogenous peptides gp350 and 4-1BBL is used to validate expression of the respective peptides in the engineered erythroid cells. Binding of an APC-labelled or PE-labelled anti-MHCI antibody is used to validate expression of the MHCI antigen presenting peptide in the engineered erythroid cells.

Functional activity of the effect of the engineered erythroid cells (gp350-HLA-A2-GPA, 4-1BBL) is assessed for their ability to stimulate the initial activation and proliferation of primary CD8+ T cells.

### Methods

Production of Lentiviral Vector

The genes for gp350-HLA-A2-GPA fusion protein and 4-1BBL are constructed. Genes encoding the proteins are cloned into the multiple cloning site of lentivirus vector pCDH with the MSCV promoter sequence from System Biosciences, such that one vector comprises the genes for both exogenous proteins. Lentivirus is produced in 293T cells by transfecting the cells with pPACKH1 (System Biosciences) and pCDH lentivirus vector containing genes for gp350-HLA-A2-GPA and 4-1BBL. Cells are placed in fresh culturing medium. The virus supernatant is collected 48 hours post-medium change by centrifugation at 1,500 rpm for 5 minutes. The supernatant is collected and frozen in aliquots at −80° C.

Transduction of Erythroid Precursor Cells

Expansion and differentiation of erythroid cells is performed according to Example 5, and transduced with the lentiviral vectors described above. Erythroid precursor cells are transduced during step 1 of the culture process. Erythroid cells in culturing medium are combined with lentiviral supernatant and polybrene. Infection is achieved by spinoculation, spinning the plate at 2000 rpm for 90 minutes at room temperature. After spinoculation, the cells are incubated at 37° C. overnight.

Antibody Binding

Binding of an APC-labelled or PE-labelled anti-gp350 antibody is used to validate expression of the gp350-HLA-A2-GPA fusion in the engineered erythroid cells. Binding of the antibody is measured by flow cytometry for APC fluorescence or PE fluorescence. A gate is set based on stained untransduced cells. Binding of an APC-labelled or PE-labelled anti-MHCI antibody is used to validate expression of the MHCI antigen presenting polypeptide in the engineered erythroid cells, as described in Example 5. Binding of an APC-labelled or PE-labelled anti-4-1BBL antibody (R&D Systems) is used to validate expression of 4-1BBL in in the engineered erythroid cells. Binding of any one of the antibodies is measured by flow cytometry for APC fluorescence or PE fluorescence. A gate is set based on stained untransduced cells.

Functional Validation Assays

The erythroid cells genetically engineered to co-express a gp350-HLA-A2-GPA fusion protein and a 4-1BBL costimulatory polypeptide are tested for their ability to stimulate the initial activation and proliferation of primary CD8+ T cells. The T cells are stimulated with the engineered erythroid cells, and the following parameters are evaluated: (1) rate of growth of the T cells stimulated with the aAPCs, as determined by a standard thymidine incorporation assay, which gives the total amount of DNA synthesized in a bulk culture; (2) induction of proliferation and cell division of CD4+ T cells; (3) viability of the CD8+ T cells stimulated by the various aAPCs during culture by fluorescent staining with annexin V and propidium iodide; (4) Bcl-xL and IL-2 expression, two genes involved in T cell survival and proliferation, respectively, using quantitative real time RT-PCR in order to determine the levels of steady-state mRNA coding for Bcl-xL and IL-2.

Cytokine Production

Cytokines are important effector molecules and provide insight into T cell differentiation. The ability of erythroid cells engineered to co-express a gp350-HLA-A2-GPA fusion and the costimulatory peptide 4-1BBL to induce certain cytokines from CD8 T cells is quantitated, for example IL-2 (a key T cell growth factor for ex vivo expansion and a cell's ability to induce IL-2 correlates well with its long term growth potential); IL-4 (a marker for TH2 differentiation); and IL-10 (an immunosuppressive cytokine that may be surrogate for T regulatory cell outgrowth). Other cytokines include, but are not limited to, TGF-β (for the same rationale as IL-10); IFN-γ (a marker for TH1 differentiation and an important effector cytokine); and TNFα (an important effector cytokine). Quantitation can be carried out using the ELISPOT (enzyme-linked immunospot) technique, that detects T cells that secrete a given cytokine (e.g., gamma interferon [IFN-γ]) in response to an antigenic stimulation. T cells are cultured with antigen-presenting cells in wells which have been coated with anti-IFN-γ antibodies. The secreted IFN-γ is captured by the coated antibody and then revealed with a second antibody coupled to a chromogenic substrate. Thus, locally secreted cytokine molecules form spots, with each spot corresponding to one IFN-γ-secreting cell. The number of spots allows one to determine the frequency of IFN-γ-secreting cells specific for a given antigen in the analyzed sample. The ELISPOT assay has also been described for the detection of tumor necrosis factor alpha, interleukin-4 (IL-4), IL-5, IL-6, IL-10, IL-12, granulocyte-macrophage colony-stimulating factor.

### Example 14. Activity of Enucleated Erythroid Cells Engineered to Co-Express an EBV-HLA-A2-GPA Fusion Protein and a Costimulatory Polypeptide in a Murine EBV Model In Vivo

Several EBV peptides have been shown to be a human leukemia antigen. Furthermore, several mouse systems have been used as disease models of EBV infection and associated diseases. The NOD/NSG EBV xenograft mouse model may be used to test an RTX-aAPC for EBV associated MS (as originally described in Fujiwara et al., 2013, Pathogens, March 14; 2(1):153-76, the entire contents of which are incorporated herein by reference) in order to test the system's ability to prohibit EBV activated autoreactive B cells. The NOD/NSG EBV xenograft model is set up using NOD/LtSz-scid IL-2rg−/−(NSG). At transplantation day, 2-5 days mice undergo 100 cGy irradiation and are transplanted intrahepatically with human CD34+ cells isolated from fetal liver or cord blood. EBV is inoculated intraperitoneally. Mice are then administered aAPC with either MHC class I or MHC class II molecules presenting EBV peptides and with a co-stimulatory molecule, (i.e. 4-1BBL). The aAPCs are either mouse RBCs clicked with the MHC molecules loaded with EBV peptide and co-stimulatory molecule, or alternatively human enucleated erythroid cells engineered to express an EBV peptide-MHC class I-GPA fusion protein and co-stimulatory molecule (e.g., the aAPC as described in Example 13). These aAPCs are expected to activate CD8 or CD4 T cells specific for the EBV peptides, which will in turn kill cells that are presenting EBV peptides through MHC class I or II. This could include depletion of B cells that have become lymphoproliferative. Animals are killed 4-10 weeks post-transfer, and EBV-specific T cell responses are analyzed using an IFN-γ ELISPOT assay as previously described (J Exp Med. 2009 Jun. 8; 206(6):1423-34, the contents of which are incorporated herein by reference). After aAPC administration, T cells are also stained for activation markers including, for example, CD62L, CD44, and/or 41BB, to evaluate phenotypes between mice with or without administration of aAPC.

### Example 15. Erythroid Cells Engineered to Present MHCI (Ovalbumin) and 4-1BBL Exhibit an In Vivo Dose Response Ova-Specific T Cells In Vivo

Murine erythroid cells were conjugated with MHCI presenting ovalbumin peptide and 4-1BBL using the click methodology (click chemistry for functionalizing erythroid cells is described in International Application No. PCT/US2018/000042, which claims priority to U.S. Provisional Application No. 62/460,589, filed Feb. 17, 2017 and U.S. Provisional Application No. 62/542,142, filed Jul. 8, 2017, incorporated by reference in their entireties herein). Briefly, mouse peripheral blood was filtered through a PAL de-leukocyte filter and labeled with 0.04 mM 6′ Azido-NHS-ester in pH8 PBS for 30 min at room temperature. Azido-labeled mRCT were incubated with 40 uM m4-1BBL-DBCO-Thiolinker and the H-2Kb SIINFEKL-DBCO-Thiolinker (SEQ ID NO: 721) monomer at room temperature for 1h and further incubated at 4 C overnight.

CD8+ T cells were purified from secondary lymphoid organs of OT1 transgenic mice using negative selection kit from Miltenyi and labeled with fluorescent dye (10 uM CFSE). 2E6 OT1 cells were injected intravenously into C57BL/6 mice on day 0. On day 0, 4 hours after OT1 injection, mice were injected intravenously with 1E6, 1E7 or 1E8 aAPC (mRCT-4-1BBL H2Kb OVA). On day 4, mice were sacrificed and single cell suspensions from their spleen and lymph nodes were stained with PE cy7 CD44, APC CD122, ad BV650 CD27, and analyzed by flow cytometry. Activation and Proliferation of OT1 T cells was monitored by following dilution of fluorescent dye signal in lymph nodes or spleen.

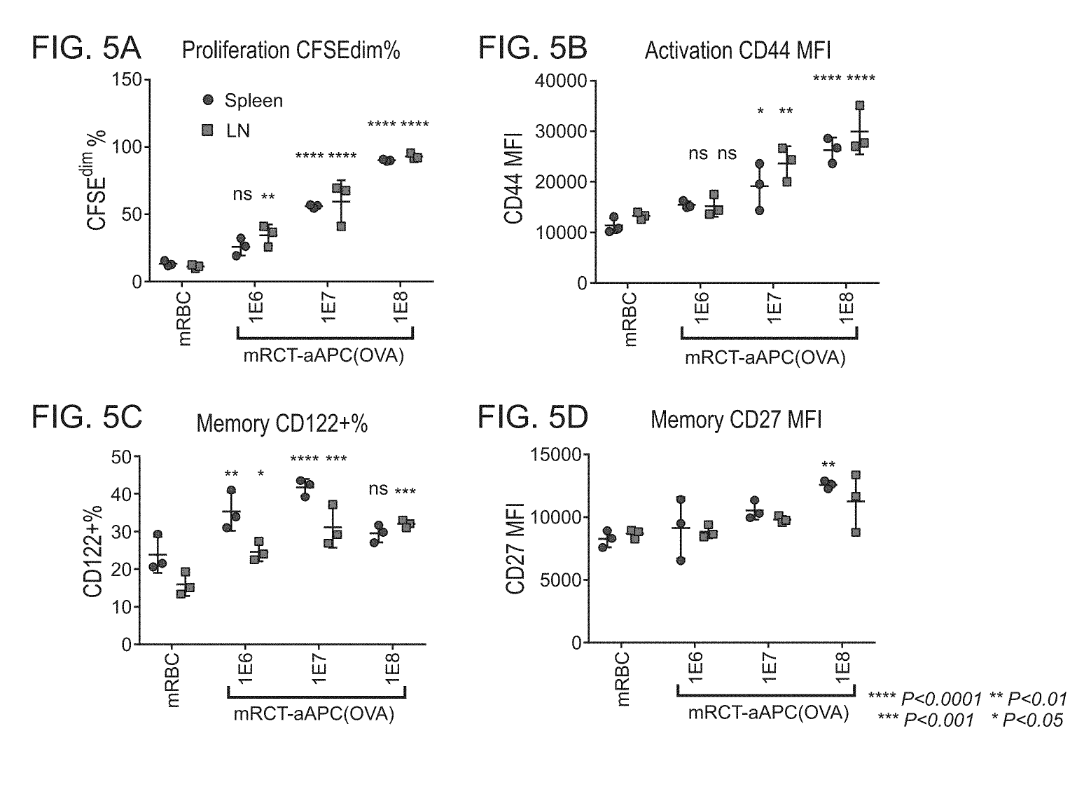

As shown in FIG. 5, murine erythroid cells presenting MHC I (ovalbumin) (mRCT-aAPC (ova) 4-1BBL), potently induce the activation and proliferation of ova-specific T cells in a dose dependent fashion in vivo, as evidenced by increased CFSE fluorescence intensity (FIG. 5A) and CD44 expression (FIG. 5B). The results also show that the OT1 T cells that were activated display a memory phenotype, as evidenced by the upregulation of key memory markers, CD122 and CD27 (FIGS. 5C and 5D), in vivo. These results demonstrate the ability to potently and selectively expand and activate antigen-specific T cells using the engineered enucleated cells as described herein.

### Example 16. A Second Dose of the Erythroid Cells Engineered to Present MHCI (Ovalbumin) and 4-1BBL Dramatically Boosts CD8+OT1 T-Cells in Both Lymph Node and Spleen

Murine erythroid cells were generated as described in Example 15. CD8+ T cells were purified from secondary lymphoid organs of OT1 transgenic mice as described in Example 15. Briefly, mice were injected intravenously with 1E9 aAPC (mRCT-4-1BBL or mRCT-4-1BBL H2Kb OVA) at day 0 and day 3. On day 6, mice were sacrificed and single cell suspensions from their spleen and lymph nodes were stained with NIR Zombie, FITC CD44, BV650 CD8 and BV785 CD62L and analyzed by flow cytometry.

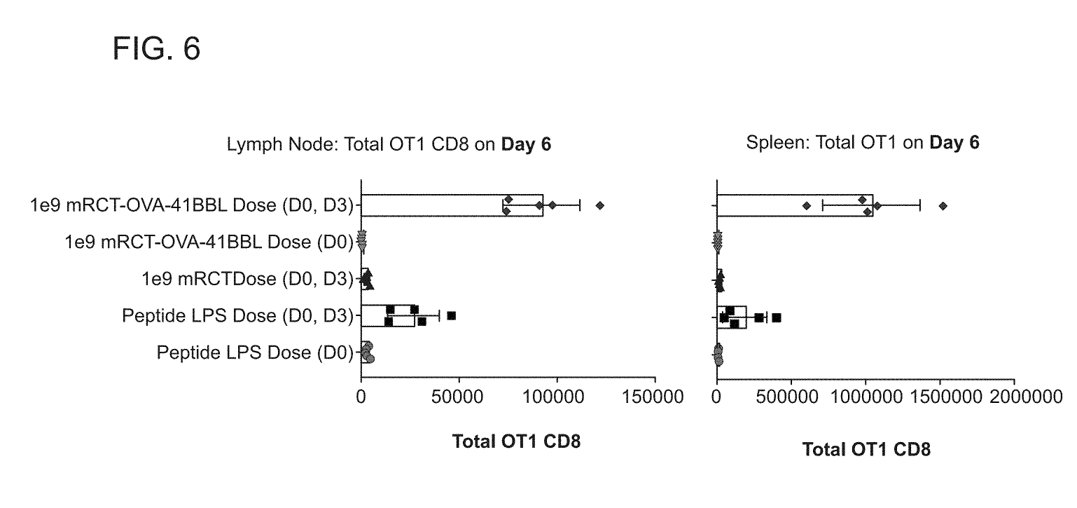

Proliferation of OT1 T cells was monitored by following dilution of fluorescent dye signal in lymph nodes or spleen. The SIINFEKL peptide (SEQ ID NO: 721) (10 μg) and adjuvant LPS (50 ng), diluted in PBS, were administered as positive controls to induce proliferation of OT-1 T cells. The results of the experiment are represented in FIG. 6, showing that a second dose of the mRCT-4-1BBL OVA on day 3 dramatically increases cell counts of the OT1-T cells, relative to a second dose of LPS and peptide on day 3. Administration of a second aAPC dose at this time drove >200-fold expansion of OT1 cells with a memory-like phenotype in the peripheral blood and secondary lymphoid organs. In contrast, a second dose of the mRCT-4-1BBL OVA on day 7 did not dramatically increase cell counts of the OT1-T cells (data not shown). It is observed that the T-cell counts are at their peak during 3-4 days after the initial administration of the RCTs. Accordingly, while not wishing to be bound by theory, it is believed that administration of a second dose during the peak of the initial OT-1 T-cell counts (between days 3 to 4), would be the most effective to induce a dramatic change in the T-cell counts.

### Example 17. Erythroid Cells Engineered to Present MHCI (Gp100) and 4-1BBL Activate Gp100-Specific T Cells In Vitro

Murine erythroid cells were conjugated with MHCI presenting tumor peptide gp100 and 4-1BBL using the click methodology (click chemistry for functionalizing erythroid cells is described in International Application No. PCT/US2018/000042, which claims priority to U.S. Provisional Application No. 62/460,589, filed Feb. 17, 2017 and U.S. Provisional Application No. 62/542,142, filed Jul. 8, 2017, incorporated by reference in their entireties herein). Briefly, mouse peripheral blood was filtered through a PAL de-leukocyte filter and labeled with 0.04 mM 6′ Azido-NHS-ester in pH8 PBS for 30 min at room temperature. Azido-labeled mRCT were incubated with 40 uM m4-1BBL-DBCO-Thiolinker at room temperature for 1h and further incubated at 4 C overnight. mRCT-m4-1BBL were clicked with biotin by incubating with 1 uM water soluble DBCO biotin at room temperature for 1h, followed by incubating with neutravidin at a 1:1 molar ratio of biotin and neutravidin. Neutravidin bound cells were then incubated with biotinylated H-2Db gp100 (KVPRNQDWL (SEQ ID NO: 722)) monomer at a 1:1 molar ratio of biotin and monomer for 1h at room temperature.

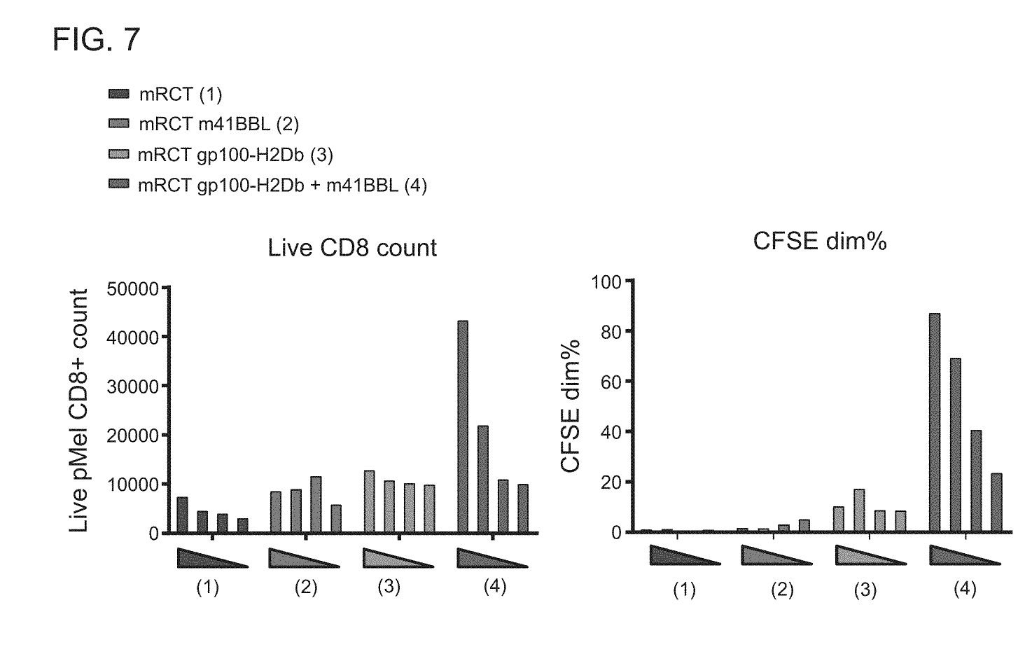

Pmel-1 cells, a transgenic CD8+ T cell population against gp100, were purified from secondary lymphoid organs mice using a negative selection kit from Miltenyi and labeled with 1 uM CFSE. 2E5 CFSE labeled primary CD8+ T cells were plated into each well of a 96 well plate. Varying amounts (3E6, 1E6, 3.3E5, or 1.1E5 cells) of mRCT, mRCT-4-1BBL, mRCT-gp100-H2Db, or mRCT-gp100-H2Db+4-1BBL were plated in cRPMI media into the wells containing CD8+ T cells at mRCT:T cell ratios of 10:1, 3.3:1, 1.1:1, and 0.37:1 (left to right in FIG. 7) and incubated for two days at 37 C. Cells were washed and stained with anti-CD8, livedead dye to quantify T cell expansion and activation.

As shown in FIG. 7, murine erythroid cells presenting MHC I (gp100) and 4-1BBL on the cell surface, potently activate gp100-specific T cells in vitro, relative to murine erythroid cells presenting MHC I (gp100) alone. These results demonstrate the ability to potently and selectively expand another type of antigen-specific T cell compared to OVA specific T cells, using the engineered enucleated cells as described herein.

### Example 18. Generation of Different Versions of HLA-A2 (HPV E7) Expressed on RCTs

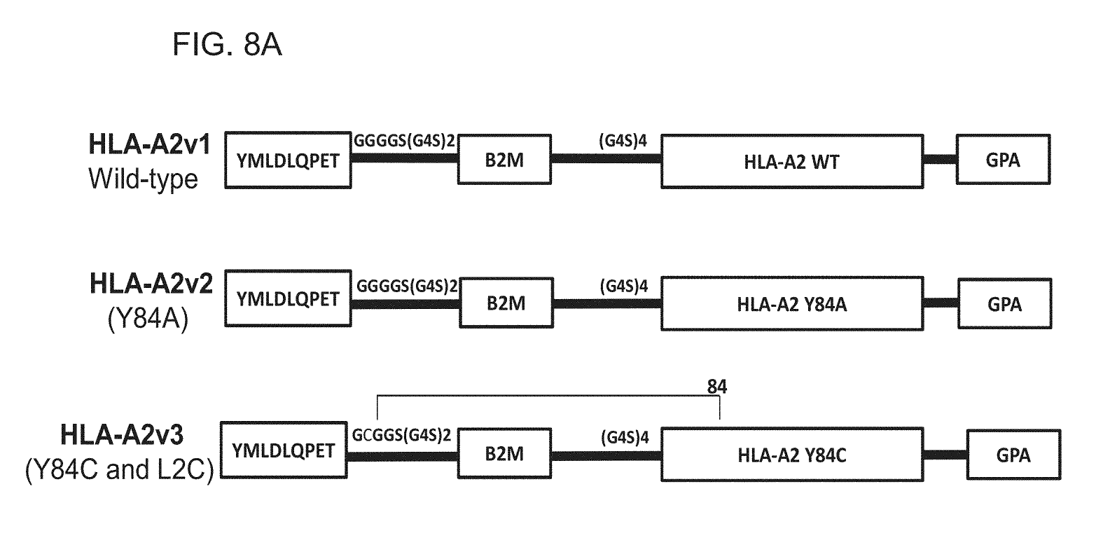

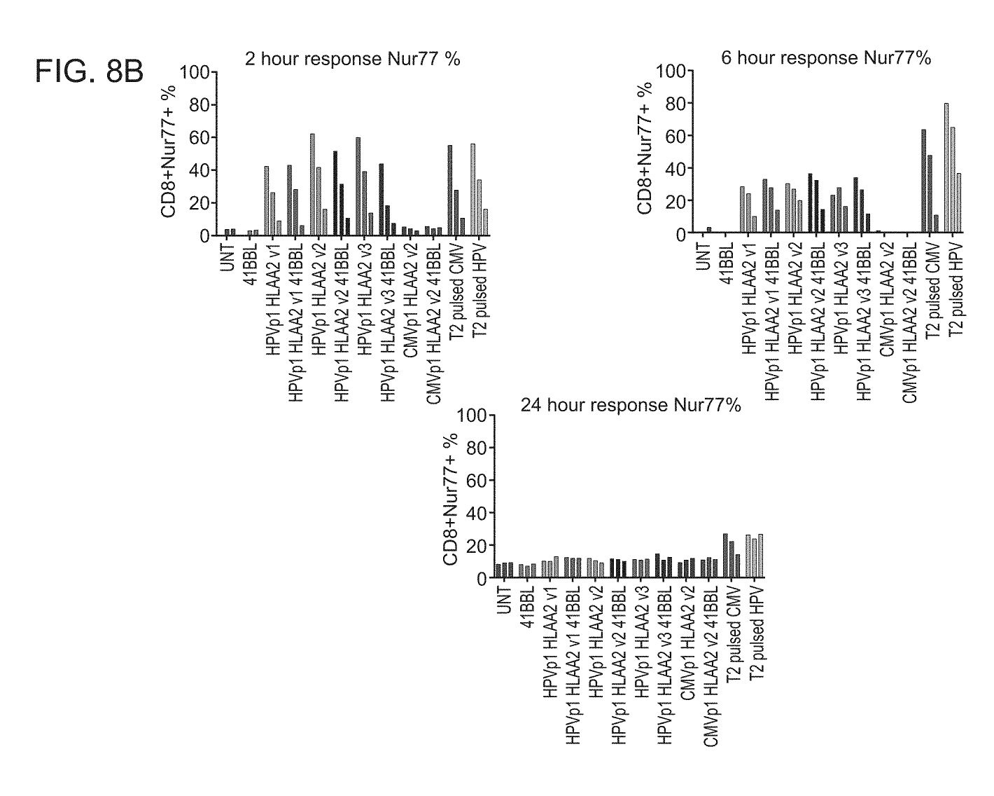

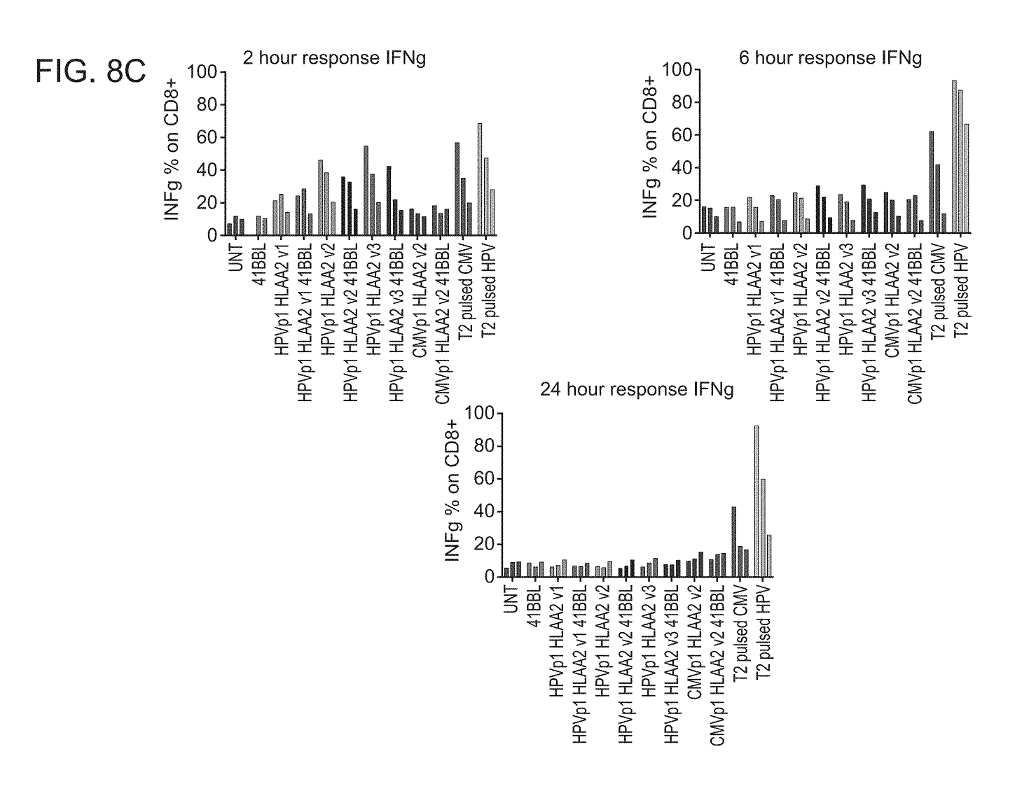

DNA constructs of three different versions of HLA-A2 (HLA-A2 wild type, HLA-A2 Y84A, and HLA-A2 Y84C+L2C) were used for expressing HPV P1 (HPV16 E7 11-20), as described in FIG. 8A and also HPV P1-2 (HPV16 E7 11-19). The sequences of these constructs are described below. See, e.g., Hansen et al., 2010, Trends Immunol. 31(10): 363-369.

HLA-A2v1 (wild type):
(SEQ ID NO: 723)
MSRSVALAVLALLSLSGLEAYMLDLQPETTGGGGSGGGGSGGGGSIQRTPKIQVYSRHP
AENGKSNFLNCYVSGFHPSDIEVDLLKNGERIEKVEHSDLSFSKDWSFYLLYYTEFTPTE
KDEYACRVNHVTLSQPKIVKWDRDMGGGGSGGGGSGGGGSGGGGSGSHSMRYFFTSV
SRPGRGEPRFIAVGYVDDTQFVRFDSDAASQRMEPRAPWIEQEGPEYWDGETRKVKAH
SQTHRVDLGTLRGYYNQSEAGSHTVQRMYGCDVGSDWRFLRGYHQYAYDGKDYIAL
KEDLRSWTAADMAAQTTKHKWEAAHVAEQLRAYLEGTCVEWLRRYLENGKETLQRT
DAPKTHMTHHAVSDHEATLRCWALSFYPAEITLTWQRDGEDQTQDTELVETRPAGDGT
FQKWAAVVVPSGQEQRYTCHVQHEGLPKPLTLRWEPSSQPTIPIGSGSGSGSEDGSGSGS
GSLSTTEVAMHTSTSSSVTKSYISSQTNDTHKRDTYAATPRAHEVSEISVRTVYPPEEETG
ERVQLAHHFSEPEITLIIFGVMAGVIGTILLISYGIRRLIKKSPSDVKPLPSPDTDVPLSSVEI
ENPETSDQ*
HLA-A2v2 (Y84A):
(SEQ ID NO: 724)
MSRSVALAVLALLSLSGLEAYMLDLQPETTGGGGSGGGGSGGGGSIQRTPKIQVYSRHP
AENGKSNFLNCYVSGFHPSDIEVDLLKNGERIEKVEHSDLSFSKDWSFYLLYYTEFTPTE
KDEYACRVNHVTLSQPKIVKWDRDMGGGGSGGGGSGGGGSGGGGSGSHSMRYFFTSV
SRPGRGEPRFIAVGYVDDTQFVRFDSDAASQRMEPRAPWIEQEGPEYWDGETRKVKAH
SQTHRVDLGTLRGAYNQSEAGSHTVQRMYGCDVGSDWRFLRGYHQYAYDGKDYIAL
KEDLRSWTAADMAAQTTKHKWEAAHVAEQLRAYLEGTCVEWLRRYLENGKETLQRT
DAPKTHMTHHAVSDHEATLRCWALSFYPAEITLTWQRDGEDQTQDTELVETRPAGDGT
FQKWAAVVVPSGQEQRYTCHVQHEGLPKPLTLRWEPSSQPTIPIGSGSGSGSEDGSGSGS
GSLSTTEVAMHTSTSSSVTKSYISSQTNDTHKRDTYAATPRAHEVSEISVRTVYPPEEETG
ERVQLAHHFSEPEITLIIFGVMAGVIGTILLISYGIRRLIKKSPSDVKPLPSPDTDVPLSSVEI
ENPETSDQ*
HLA-A2v3 (Y84C and L2C):
(SEQ ID NO: 725)
MSRSVALAVLALLSLSGLEAYMLDLQPETTGCGGSGGGGSGGGGSIQRTPKIQVYSRHP
AENGKSNFLNCYVSGFHPSDIEVDLLKNGERIEKVEHSDLSFSKDWSFYLLYYTEFTPTE
KDEYACRVNHVTLSQPKIVKWDRDMGGGGSGGGGSGGGGSGGGGSGSHSMRYFFTSV
SRPGRGEPRFIAVGYVDDTQFVRFDSDAASQRMEPRAPWIEQEGPEYWDGETRKVKAH
SQTHRVDLGTLRGCYNQSEAGSHTVQRMYGCDVGSDWRFLRGYHQYAYDGKDYIAL
KEDLRSWTAADMAAQTTKHKWEAAHVAEQLRAYLEGTCVEWLRRYLENGKETLQRT
DAPKTHMTHHAVSDHEATLRCWALSFYPAEITLTWQRDGEDQTQDTELVETRPAGDGT
FQKWAAVVVPSGQEQRYTCHVQHEGLPKPLTLRWEPSSQPTIPIGSGSGSGSEDGSGSGS
GSLSTTEVAMHTSTSSSVTKSYISSQTNDTHKRDTYAATPRAHEVSEISVRTVYPPEEETG
ERVQLAHHFSEPEITLIIFGVMAGVIGTILLISYGIRRLIKKSPSDVKPLPSPDTDVPLSSVEI
ENPETSDQ*
These 3 versions of HLA-A2 were used to express the single chain
variant HPV P1-2-HLA-A2-GPA:
(SEQ ID NO: 726)
MSRSVALAVLALLSLSGLEAYMLDLQPETGGGGSGGGGSGGGGSIQRTPKIQVYSRHPA
ENGKSNFLNCYVSGFHPSDIEVDLLKNGERIEKVEHSDLSFSKDWSFYLLYYTEFTPTEK
DEYACRVNHVTLSQPKIVKWDRDMGGGGSGGGGSGGGGSGGGGSGSHSMRYFFTSVS
RPGRGEPRFIAVGYVDDTQFVRFDSDAASQRMEPRAPWIEQEGPEYWDGETRKVKAHS
QTHRVDLGTLRGYYNQSEAGSHTVQRMYGCDVGSDWRFLRGYHQYAYDGKDYIALK
EDLRSWTAADMAAQTTKHKWEAAHVAEQLRAYLEGTCVEWLRRYLENGKETLQRTD
APKTHMTHHAVSDHEATLRCWALSFYPAEITLTWQRDGEDQTQDTELVETRPAGDGTF
QKWAAVVVPSGQEQRYTCHVQHEGLPKPLTLRWEPSSQPTIPIGSGSGSGSEDGSGSGS
GSLSTTEVAMHTSTSSSVTKSYISSQTNDTHKRDTYAATPRAHEVSEISVRTVYPPEEETG
ERVQLAHHFSEPEITLIIFGVMAGVIGTILLISYGIRRLIKKSPSDVKPLPSPDTDVPLSSVEI
ENPETSDQ

Production of Lentiviral Vector

The genes for HPV P1-HLA-A2-GPA fusion protein and 4-1BBL were constructed. Genes encoding the proteins were cloned into the multiple cloning site of lentivirus vector pCDH with the MSCV promoter sequence from System Biosciences, such that one vector comprised the genes for both exogenous proteins. Lentivirus was produced in 293T cells by transfecting the cells with pPACKH1 (System Biosciences) and pCDH lentivirus vector containing genes for HPV P1-HLA-A2-GPA and 4-1BBL. Alternately separate vectors containing genes for HPV P1-HLA-A2-GPA and 4-1BBL can be utilized. Cells were placed in fresh culturing medium. The virus supernatant was collected 48 hours post-medium change by centrifugation at 1,500 rpm for 5 minutes. The supernatant was collected and frozen in aliquots at −80° C.

### Transduction of Erythroid Precursor Cells

Expansion and differentiation of erythroid cells was performed according to Example 5, with the lentiviral vectors described above. Erythroid precursor cells were transduced during step 1 of the culture process. Erythroid cells in culturing medium were combined with lentiviral supernatant and polybrene. Infection was achieved by spinoculation, spinning the plate at 2000 rpm for 90 minutes at room temperature. After spinoculation, the cells were incubated at 37° C. overnight

Antibody Binding

Binding of an APC-labelled or PE-labelled anti-beta 2 microglobulin antibody was used to validate expression of the HPV P1-HLA-A2-GPA fusion in the engineered erythroid cells, as described in Example 5. Binding of an APC-labelled or PE-labelled anti-MHCI antibody was used to validate expression of the MHCI antigen presenting polypeptide in the engineered erythroid cells, also as described in Example 5. Binding of an APC-labelled or PE-labelled anti-4-1BBL antibody (R&D Systems) was used to validate expression of 4-1BBL in the engineered erythroid cells. Binding of any one of the antibodies was measured by flow cytometry for APC fluorescence or PE fluorescence. A gate was set based on stained untransduced cells.

### Example 19: Activity of HLA-A2 (HPV E7) Expressed on RCTs in Stimulating HPV-Specific T Cells In Vitro

Human RCTs expressing 4-1BBL, HLA-A2 HPV P1, or both 4-1BBL and HLA-A2 HPV P1 were prepared essentially as described in Example 18. These RCTs co-expressing a HPV P1-HLA-A2-GPA fusion protein and a 4-1BBL costimulatory polypeptide were tested for their ability to stimulate the initial activation and proliferation of primary CD8+ T cells, essentially as described in Example 6. T2 cells pulsed with the CMV or HPV peptide were utilized as positive controls.

Primary human CD8+ cells were purchased from Astarte Biologics. Varying amounts human RCTs, were plated in cRPMI media into the wells containing 4E4 or 2E4 CD8+ T cells at RCT:T cell ratios of 10:1, 2:1 and 0.4:1, (left to right, for each RCT, in FIGS. 8B and 8C) and incubated for two days at 37 C.

Intracellular staining was utilized to measure cellular levels of Nur77 and IFN-γ. Briefly, the cells were fixed with 200 μL of IC Fixation Buffer and incubated 20-60 minutes in dark, at room temperature. Samples were centrifuged at 400-600×g at room temperature for 5 minutes. The supernatant was discarded and 200 μL of permeabilization buffer was added. Samples were centrifuged at 400-600×g at room temperature for 5 minutes. The supernatant was discarded and the pellet was resuspended in about 100 μL with 1× permeabilization buffer. Without washing, directly conjugated antibody (anti-Nur77 PE and anti-IFN-γ APC) was added followed by incubation for at least 30 minutes in dark, at room temperature. Nur77 and IFN-γ were analyzed at 2, 6 and 24 hours after incubation of the RCTs with the primary CD8+ T-cells.

Flow cytometry analysis suggested that all the tested versions of HLA-A2 (HPV E7) were well-expressed on the surface of human RCTs (data not shown). Furthermore, all three versions lead to the activation of HPV E7 specific T cells, as determined by Nur77 expression (FIG. 8B), and IFNγ secretion (FIG. 8C). These results demonstrate the ability to potently and selectively activate an antigen-specific T cell, for example HPV specific T cells, using the engineered enucleated cells as described herein.

### Example 20. Ovalbumin-Specific T Cells Expanded and Activated by Erythroid Cells Presenting MHCI (Ovalbumin) and 4-1BBL Reduce Tumor Volume

Murine erythroid cells were conjugated with MHCI presenting ovalbumin peptide and 4-1BBL using the click methodology, as described in Example 15. Briefly, mouse peripheral blood was filtered through a PAL de-leukocyte filter and labeled with 0.04 mM 6′ Azido-NHS-ester in pH8 PBS for 30 min at room temperature. Azido-labeled mRCT were incubated with 40 μM m4-1BBL-DBCO-Thiolinker and 40 μM H-2Kb SIINFEKL-DBCO-Thiolinker monomer (SEQ ID NO: 721) at room temperature for 1h and then further incubated at 4° C. overnight.

CD8+ T cells were purified from secondary lymphoid organs of OT1 transgenic mice using negative selection kit from Miltenyi and labeled with 10 uM Cell-Trace Violet. 2×10⁶ tumor cells expressing ovalbumin (EG7.OVA) were injected subcutaneously (s.c.) into B6 Cd45.1 mice 7 days before dosing. Experimental groups of mice (7 per group) were as follows:
(1) OT1 dose: 2×10⁶ OT1 (i.v.); cell dose: 1×10⁹ murine RCT (mRCT) (i.v.);
(2) OT1 dose: 2×10⁶ OT1 (i.v.); cell dose: 1×10⁹ murine RCT presenting MHCI (ovalbumin) and 4-1BBL (mRCT-OVA-4-1BBL) (i.v.).

The first day of dosing with OT1 and mRCTs was determined based on tumor volume. At day 0, tumors were randomized on tumor volumes of 60-193 mm³, with an average volume of 141 mm³. Adoptive T cell transfer of OT1 at the doses set out in groups (1) and (2) above was carried out by intravenous injection into wild type mice (day 1). Mice were then dosed with 1×10⁹ mRCT or mRCT-OVA-41-BBL as set out in groups (1) and (2) above, at days 1, 4, and 8. Significant OT1 proliferation was observed 3-4 days post-dosing as measured by CTV dilution. The second dose was given at 4 days after the first dose, corresponding to the time of maximum OT1 cell expansion. Tumor volume was measured over days 1-14, and mice were euthanized once tumor volumes reached greater than 2000 mm³.

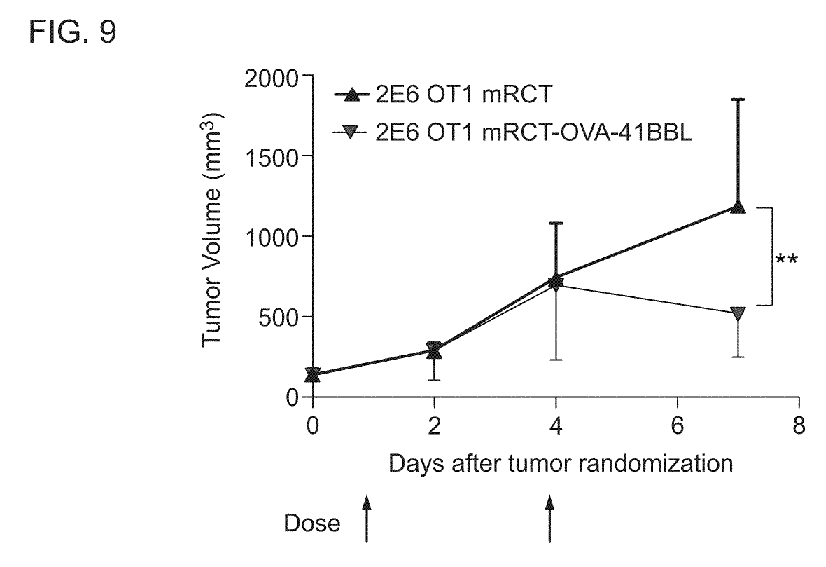

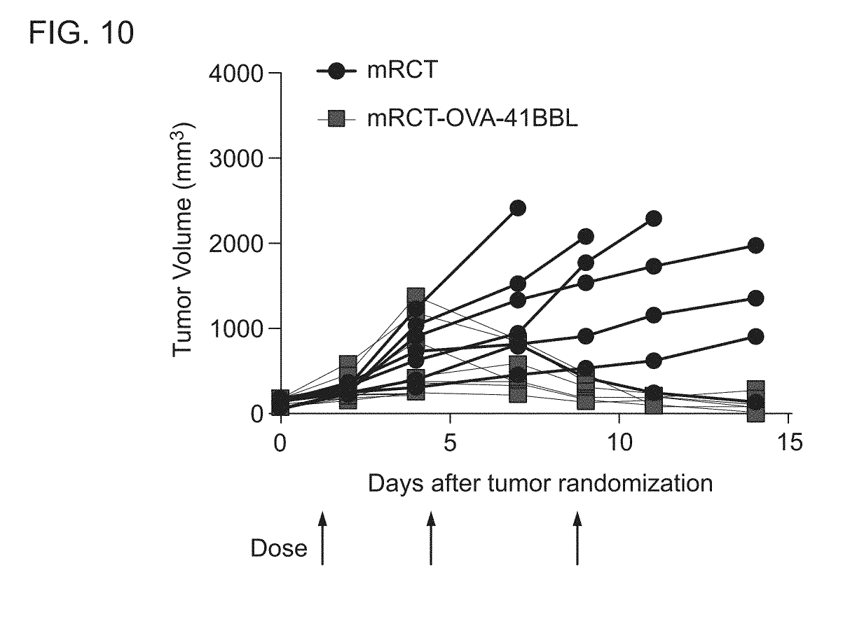

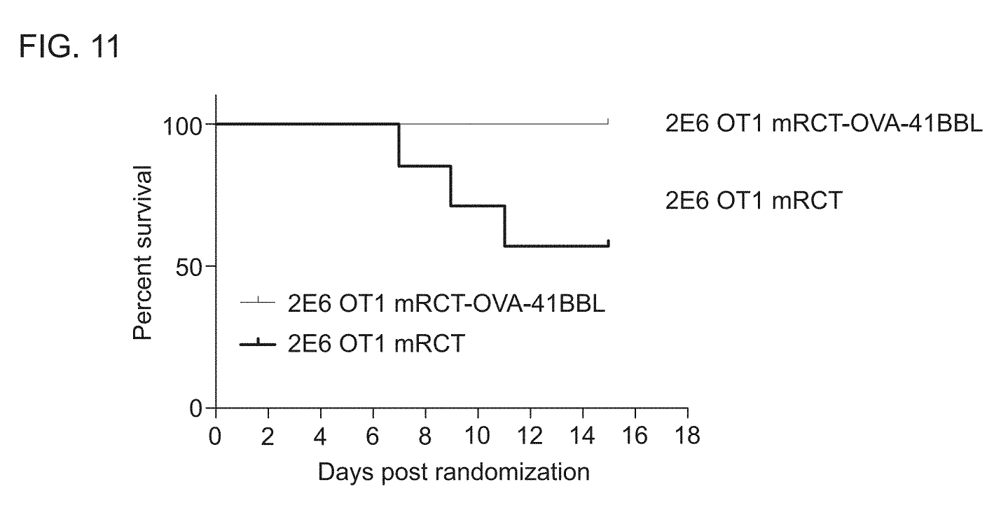

The results of this experiment are shown in FIGS. 9-11. FIG. 9 shows the change in average tumor volume (mm³) over time after tumor randomization, where dosing was carried out at days 1, 4 and 8. FIG. 10 shows the individual tumor volume (mm³) over time after tumor randomization, where dosing was carried out at days 1, 4 and 8. The results in FIG. 9 and FIG. 10 show that mRCT-OVA-4-1BBL were able to activate tumor specific T cells (OT1) at a dose of 2×10⁶, as shown by a reduction of EG7.OVA tumor volume over time as compared to mRCTs. Administration of aAPC mRCT-OVA-4-1BBL to mice bearing EG7.OVA tumors caused 60% tumor growth inhibition by Day 7 after dosing compared to controls, which corresponded with the increased expansion of the OT1s (FIG. 9). FIG. 11 is a graph showing percent survival of mice over time. As shown in FIG. 11, 7 out of 7 mice dosed with mRCT-OVA-4-1BBL survived by day 14 post-randomization, while only 4 out of 7 mice dosed with mRCT survived by day 14 post-randomization. In addition, in a separate, similar experiment it was observed that all aAPC dosed mice demonstrated increased OT1 infiltration into EG7.OVA tumors with some tumors additionally demonstrating endogenous OVA specific T cell infiltration (data not shown).

This experiment was repeated, generally as described above, except that adoptive T cell transfer of OT1 was carried out at a dose of 5×10⁵ OT1 (i.v.), and in addition to a group of mice dosed with mRCT-OVA-4-1BBL, the following additional control groups were included (12 mice per group): phosphate buffered saline (PBS), SIINFEKL peptide (SEQ ID NO: 721) and Lipopolysaccharide (LPS), mRCT-OVA, and mRCT-4-1BBL. In this experiment, 2×10⁶ tumor cells expressing ovalbumin (EG7.OVA) were injected subcutaneously (s.c.) into wild type mice at day −9, tumor randomization was on day 0 when the tumor volume was between 52-130 mm³, and at an average of 97 mm³, OT1 dosing was on day 1, and mRCT dosing was on days 1, 5 and 9.

The results from these experiments (data not shown) demonstrated that, similar to the first experiment, mRCT-OVA-4-1BBL activated tumor specific T cells (OT1), as evidenced by a reduction of EG7.OVA tumor volume over time as compared to that of the control mRCTs, PBS and peptide+LPS. No significant differences were observed among these controls. Further, no reduction in tumor volume was observed for mRCT-OVA and mRCT-4-1BBL as compared to that of the control mRCT. These results indicate that the combination of OVA and 4-1BBL on the mRCT were necessary for the observed tumor control.

### Example 21. Generation of Erythroid Cells Presenting MHCI (Ovalbumin), 4-1BBL and IL-15 (OVA-H2Kb+4-1BBL+IL15)

Murine erythroid cells were conjugated with each of MHCI presenting ovalbumin peptide (OVA-H2Kb), 4-1BBL and IL-15 using a modification of the click methodology (click chemistry for functionalizing erythroid cells is described in International Application No. PCT/US2018/000042, which claims priority to U.S. Provisional Application No. 62/460,589, filed Feb. 17, 2017 and U.S. Provisional Application No. 62/542,142, filed Jul. 8, 2017, incorporated by reference in their entireties herein). Here, conjugation of three peptides onto the murine erythroid cells was achieved using two different handles, dibenzocyclooctyne Group (DBCO) and trans-cyclooctene (TCO). Triple click cells were produced with the following reagents: Fc-hIL15TP (TCO labeled); Fc-hIL-15TP (DBCO labeled); m4-1BBL (DBCO thiolinker) and OVA-H2Kb (DBCO thiolinker). Briefly, the reactions were performed as follows:

OVA-4-1BBL-IL15 (MTZ-TCO)

Mouse peripheral blood was filtered through a PAL de-leukocyte filter and 1×10⁸ mRCT were labeled with 0.04 mM 6′ Azido-NHS-ester and 0.04 mM methyltetrazine-PEG-NHS-ester (MTZ) in pH8 PBS for 30 min at room temperature. 5 ul of 20 μM Fc-hIL-15TP (TCO labeled) and 5 ul of 50 μM OVA-H2Kb-DBCO-thiolinker were added and incubated for 1 hr at room temp. 5 ul of 50 μM m4-1BBL-DBCO-thiolinker was added to the cells and the mixture was incubated overnight at 4° C.

OVA-4-1BBL-IL15 (AZ-DBCO)

Mouse peripheral blood was filtered through a PAL de-leukocyte filter and 1×10⁸ mRCT were labeled with 0.04 mM 6′ Azido-NHS-ester in pH8 PBS for 30 min at room temperature. Azido-labeled mRCT were incubated with 5 ul of 20 μM Fc-hIL-15TP-DBCO and 5 ul of 50 μM OVA-H2Kb-DBCO-thiolinker for 1 hr at room temp. 5 ul of 50 μM m4-1BBL-DBCO-thiolinker was added to the cells and the mixture was incubated overnight at 4° C.

OVA-4-1BBL

Mouse peripheral blood was filtered through a PAL de-leukocyte filter and 1×10⁸ mRCT were labeled with 0.04 mM 6′ Azido-NHS-ester in pH8 PBS for 30 min at room temperature. Azido-labeled mRCT were incubated with 5 ul of 50 μM m4-1BBL-DBCO-thiolinker and 5 ul of 50 μM OVA-H2Kb-DBCO-thiolinker and the mixture was incubated overnight at 4° C.

Copy number of IL15, OVA and 4-1BBL was determined for the mRCTs triple clicked with OVA-4-1BBL-IL15 (MTZ-TCO) or OVA-4-1BBL-IL15 (AZ-DBCO), and mRCTs double clicked with OVA-4-1BBL. While the copy number for each of OVA and 4-1BBL was similar among the three groups, mRCTs triple clicked with OVA-4-1BBL-IL15 (MTZ-TCO) showed considerably higher IL15 copy number as compared to mRCTs triple clicked with OVA-4-1BBL-IL15 (AZ-DBCO) (see Table 27 below).

TABLE 27
Copy Number

| | IL15 | OVA | 4-1BBL |
|---|---|---|---|
| Group | copy # | copy # | copy # |
| OVA-4-1BBL-IL15 | 370,231 | 7,270 | 128,194 |
| (MTZ-TCO) | | | |
| OVA-4-1BBL-IL15 | 31,544 | 10,345 | 118,819 |
| (AZ-DBCO) | | | |
| OVA-4-1BBL | — | 29,958 | 144,952 |

While not wishing to be bound by theory, it is believed that mRCTs triple clicked with the alternative click handle (MTZ-TCO) bypassed potential competition with DBCO to achieve the higher copy number of IL-15.

### Example 22: Erythroid Cells Presenting MHCI (Ovalbumin), 4-1BBL and IL-15 (OVA-H2Kb+4-1BBL+IL15) Show Increased OT1 CD8+ T Cell Proliferation Over Cells Presenting MHCI (Ovalbumin) and 4-1BBL (OVA-H2Kb+4-1BBL)

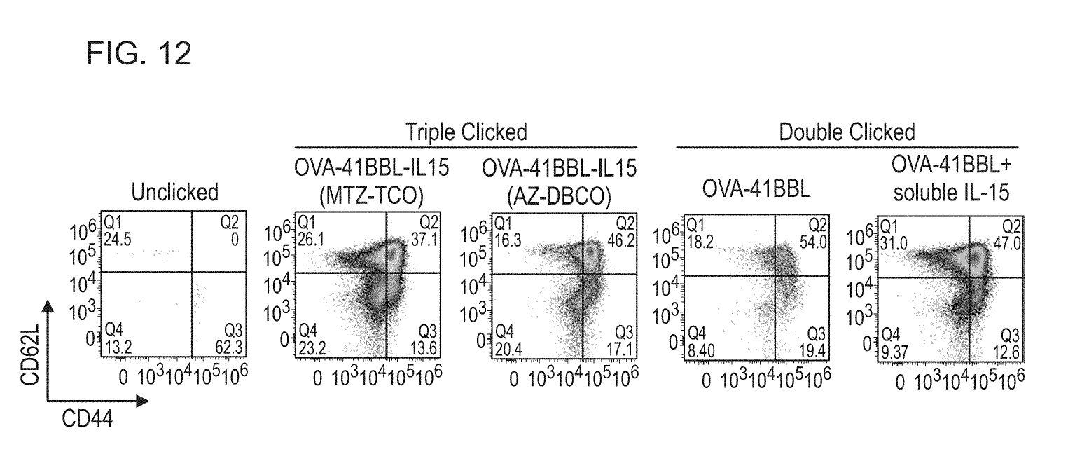

Murine RCTs were prepared as described in Example 20. CD8+ T cells were purified from the spleen of OT1 transgenic mice using a negative selection kit from Miltenyi and labeled with 10 uM CFSE. 2E5 CFSE labeled OT1 CD8+ T cells were plated into each well of a 96 well plate. Unclicked mRCTs, triple clicked mRCTs (OVA-4-1BBL-IL15 (MTZ-TCO) and OVA-4-1BBL-IL15 (AZ-DBCO)), or double clicked mRCTs (OVA-4-1BBL, and OVA-4-1BBL+ soluble IL-15 (20 ng/ml) and OT1 CD8+ T cells were co-incubated at ratios of 25:1, 10:1, and 4:1 (mRCT:OT1) and incubated for 4 days at 37 C and 5% CO₂. On day 4, cells were stained with Pacific blue CD44 and BV785 CD62L and analyzed by flow cytometry. CD44 was used as a marker of CD8+ T cell proliferation. The results of this experiment are shown in FIG. 12. The results demonstrated that mRCTs triple clicked with OVA-4-1BBL-IL15 (MTZ-TCO) drove increased OT1 CD8+ T cell proliferation by Day 4 as compared to mRCT-OVA-4-1BBL (double clicked), and that the mRCTs triple clicked with OVA-4-1BBL-IL15 (MTZ-TCO) drove similar OT1 CD8+ T cell proliferation by Day 4 as mRCT OVA-4-1BBL (double clicked) in combination with soluble IL15. As shown in FIG. 12, second panel from left, mRCTs triple clicked with the alternative click handle (MTZ-DBCO) also drove OT1 CD8+ T cell proliferation by Day 4.

OT1 CD8+ T cell proliferation at day 4 was also determined. The results, as shown in FIG. 13, demonstrate that mRCTs triple clicked with OVA-4-1BBL-IL15 (MTZ-TCO) stimulated substantial OT1 CD8+ T cell proliferation, and to a similar level as that achieved with the combination of mRCT OVA-4-1BBL (double clicked) and soluble IL-15. Further, mRCTs triple clicked with OVA+4-1BBL+IL15 (MTZ-TCO) increased proliferation of OT1 CD8+ T cells to a greater level than mRCTs triple clicked with the DBCO handle, OVA-H2Kb+4-1BBL (AZ-DBCO). As shown above in Table 27, mRCTs triple clicked with OVA-4-1BBL-IL15 (MTZ-TCO) showed a higher IL15 copy number as compared to mRCTs triple clicked with OVA-4-1BBL-IL15 (AZ-DBCO), indicating that here, the observed increased proliferation of OT1 CD8+ T cells was due, at least in part, to increased IL15 copy number. In addition, the percentages of effector T cell and memory T cell in the OT1 CD8+ T cell populations were determined. It was found that the percent of CD8+ T cells that were effector or memory T cells did not differ significantly among the double clicked mRCTs and triple clicked mRCTs groups (data not shown).

In summary, the results from this experiment show that a combination of signals 1, 2 and 3 on RBCs, specifically a combination of OVA, 4-1BBL and IL-15, promotes greater CD8+ T cell proliferation than signals 1 and 2, i.e., OVA and 4-1BBL, alone.

### Example 23. Generation of Erythroid Cells Presenting MHCI (Ovalbumin), 4-1BBL and IL-12 (OVA-H2Kb+4-1BBL+IL12)

Murine erythroid cells were conjugated with each of MHCI presenting ovalbumin peptide (OVA-H2Kb), 4-1BBL and IL-12 using a modification of the click methodology (click chemistry for functionalizing erythroid cells is described in International Application No. PCT/US2018/000042, which claims priority to U.S. Provisional Application No. 62/460,589, filed Feb. 17, 2017 and U.S. Provisional Application No. 62/542,142, filed Jul. 8, 2017, incorporated by reference in their entireties herein). Triple click cells were produced with the following reagents: Fc-mIL12Fc (TCO-PEG4-NHS Ester labeled); m4-1BBL (DBCO thiolinker) and OVA-H2Kb (DBCO thiolinker). Briefly, the reactions were performed as follows:

OVA-4-1BBL-IL12 (MTZ-TCO)

Mouse peripheral blood was filtered through a PAL de-leukocyte filter and 1×10⁸ mRCT were labeled with 0.04 mM 6′ Azido-NHS-ester and 0.02 mM methyltetrazine-PEG-NHS-ester (MTZ) in pH 9 PBS for 30 min at room temperature. 5 ul of 20 μM Fc-mIL12Fc-TCO-PEG4-NHS Ester and 5 ul of 50 μM OVA-H2Kb-DBCO-thiolinker were added and incubated for 1 hr at room temp. 5 ul of 50 μM m4-1BBL-DBCO-thiolinker was added to the cells and the mixture was incubated overnight at 4° C.

OVA-4-1BBL

Mouse peripheral blood was filtered through a PAL de-leukocyte filter and 1×10⁸ mRCT were labeled with 0.04 mM 6′ Azido-NHS-ester and 0.02 mM methyltetrazine-PEG-NHS-ester (MTZ) in pH 9 PBS for 30 min at room temperature. Azido-labeled mRCT were incubated with 5 ul of 50 μM m4-1BBL-DBCO-thiolinker and 5 ul of 50 μM OVA-H2Kb-DBCO-thiolinker and the mixture was incubated overnight at 4° C.

Copy number of IL12, OVA and 4-1BBL was determined for the mRCTs triple clicked with OVA-4-1BBL-IL12, and mRCTs double clicked with OVA-4-1BBL. (see Table 28 below).

TABLE 28
Copy Number

| | IL12 | OVA | 4-1BBL |
|---|---|---|---|
| Group | copy # | copy # | copy # |
| OVA-4-1BBL-IL12 | 90,277 | 12,451 | 72,579 |
| OVA-4-1BBL | — | 26,397 | 105,807 |

### Example 24. Generation of Erythroid Cells Presenting MHCI (Ovalbumin), 4-1BBL and IL-7 (OVA-H2Kb+4-1BBL+IL7)

Murine erythroid cells were conjugated with each of MHCI presenting ovalbumin peptide (OVA-H2Kb), 4-1BBL and IL-7 using a modification of the click methodology (click chemistry for functionalizing erythroid cells is described in International Application No. PCT/US2018/000042, which claims priority to U.S. Provisional Application No. 62/460,589, filed Feb. 17, 2017 and U.S. Provisional Application No. 62/542,142, filed Jul. 8, 2017, incorporated by reference in their entireties herein). Triple click cells were produced with the following reagents: Fc-mIL-7 (DBCO-Sulfo-NHS ester labeled); m4-1BBL (DBCO thiolinker) and OVA-H2Kb (DBCO thiolinker). Briefly, the reactions were performed as follows:

OVA-4-1BBL-IL7 (AZ-DBCO)

Mouse peripheral blood was filtered through a PAL de-leukocyte filter and 1×10⁸ mRCT were labeled with 0.04 mM 6′ Azido-NHS-ester and 0.02 mM methyltetrazine-PEG-NHS-ester (MTZ) in pH 9 PBS for 30 min at room temperature. Azido-labeled mRCT were incubated with 5 ul of 20 μM Fc-mIL-7-DBCO-Sulfo-NHS ester and 5 ul of 50 μM OVA-H2Kb-DBCO-thiolinker for 1 hr at room temp. 5 ul of 50 μM m4-1BBL-DBCO-thiolinker was added to the cells and the mixture was incubated overnight at 4° C.

OVA-4-1BBL

Mouse peripheral blood was filtered through a PAL de-leukocyte filter and 1×10⁸ mRCT were labeled with 0.04 mM 6′ Azido-NHS-ester and 0.02 mM methyltetrazine-PEG-NHS-ester (MTZ) in pH 9 PBS for 30 min at room temperature. Azido-labeled mRCT were incubated with 5 ul of 50 μM m4-1BBL-DBCO-thiolinker and 5 ul of 50 μM OVA-H2Kb-DBCO-thiolinker and the mixture was incubated overnight at 4° C.

Copy number of IL7, OVA and 4-1BBL was determined for the mRCTs triple clicked with OVA-4-1BBL-IL7, and mRCTs double clicked with OVA-4-1BBL. (see Table 29 below).

TABLE 29
Copy Number

| | | OVA | 4-1BBL |
|---|---|---|---|
| Group | IL7 copy # | copy # | copy # |
| OVA-4-1BBL-IL7 | 75,170 | 10,660 | 75,106 |
| OVA-4-1BBL | — | 26,397 | 105,807 |

### Example 25: Erythroid Cells Presenting MHCI (Ovalbumin) and 4-1BBL with IL-7 (OVA-H2Kb+4-1BBL+IL7), IL-12 (OVA-H2Kb+4-1BBL+IL12), or IL-15 (OVA-H2Kb+4-1BBL+IL15), Show Increased OT1 CD8+ T Cell Proliferation Over Cells Presenting 4-1BBL (OVA-H2Kb+41-BB1)

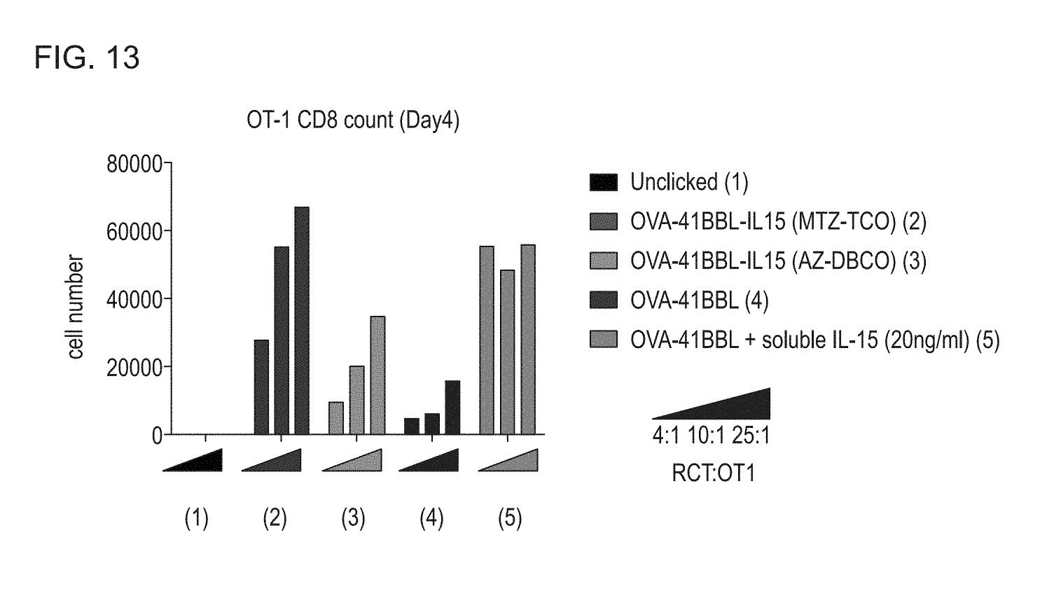

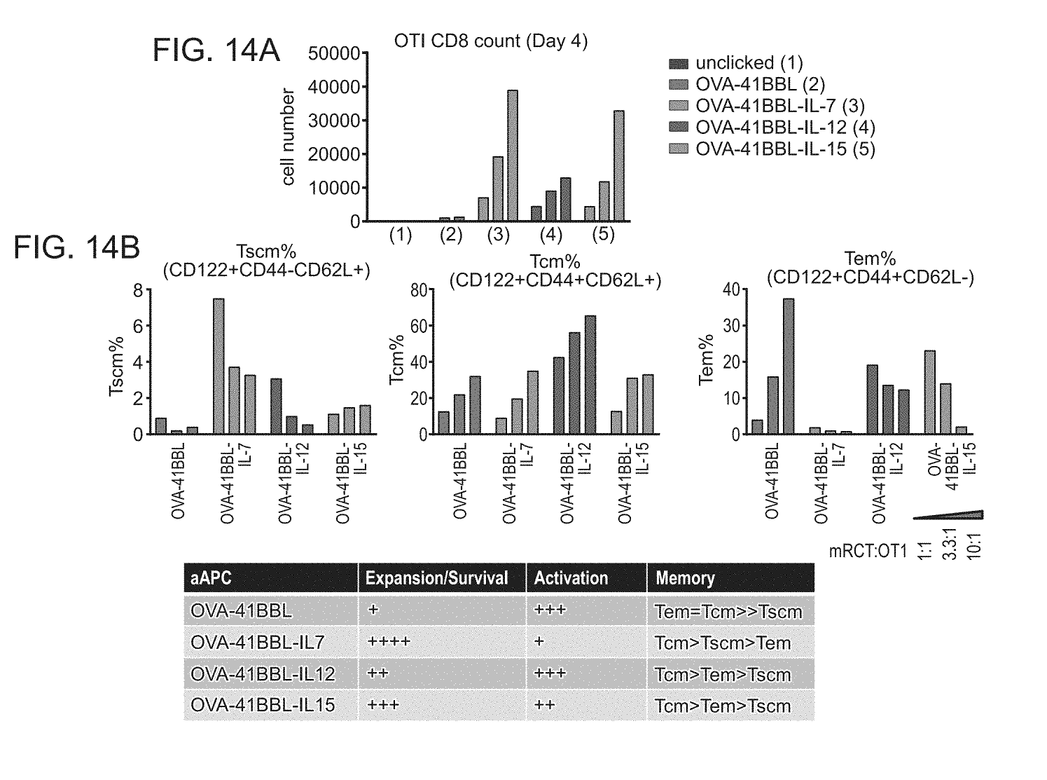

Murine RCTs were prepared, for example as described in Examples 21, 23 and 24. CD8+ T cells were purified from the spleen and lymph nodes of OT1 transgenic mice using a negative selection kit from Miltenyi and labeled with 1 uM cell trace violet. 2E5 cell trace violet labeled OT1 CD8+ T cells were plated into each well of a 96 well plate. Unclicked mRCTs or triple clicked mRCTs (OVA-4-1BBL-IL7, OVA-4-1BBL-IL12, or OVA-4-1BBL-IL15), and OT1 CD8+ T cells were co-incubated at ratios of 1:1.1, 1:3.3, and 1:10 (mRCT:OT1) and incubated for 4 days at 37 C and 5% CO₂. On day 4, proliferation, activation and memory markers of OT1 T cells were analyzed by multi-color flow cytometry. Cells were stained with FITC CD44, APC CD122, and BV785 CD62L. The results of this experiment are shown in FIG. 14. The results demonstrated that the mRCTs triple clicked with OVA-4-1BBL-IL7, OVA-4-1BBL-IL12, or OVA-4-1BBL-IL15 drove increased OT1 CD8+ T cell proliferation by Day 4 as mRCT OVA-4-1BBL (double clicked) (FIG. 14A). In addition, the percentages of memory stem T cells (Tscm), central memory T cells (Tcm) and effector memory T cells (Tem) phenotypes, in the OT1 CD8+ T cell populations, were determined. It was found that the mRCTs triple clicked with OVA-4-1BBL-IL7, OVA-4-1BBL-IL12, or OVA-4-1BBL-IL15 drove increased activation of a Tcm phenotype, whereas mRCTs double clicked with mRCT OVA-4-1BBL drove nearly equal Tem and Tcm phenotypes (FIG. 14B).

In summary, the results from this experiment show that a combination of signals 1, 2 and 3 on RBCs, specifically a combination of OVA-4-1BBL-IL7, OVA-4-1BBL-IL12, or OVA-4-1BBL-IL15, promotes greater CD8+ T cell proliferation than signals 1 and 2, i.e., OVA and 4-1BBL, alone.

### Example 26: Mice Treated with Erythroid Cells Presenting MHCI (Ovalbumin) and 4-1BBL Demonstrate EG7.OVA Tumor Control Even Upon being Re-Challenged with EG7.OVA Tumor Cells

Murine erythroid cells were conjugated with MHCI presenting ovalbumin peptide and 4-1BBL using the click methodology, as described in Example 15. Briefly, mouse peripheral blood was filtered through a PAL de-leukocyte filter and labeled with 0.04 mM 6′ Azido-NHS-ester in pH9 PBS for 30 min at room temperature. Azido-labeled mRCT were incubated with 40 μM m4-1BBL-DBCO-Thiolinker and 5 ul of 50 μM OVA-H2Kb-DBCO-thiolinker at room temperature for 1h and then further incubated at 4° C. overnight. CD8+ T cells were purified from secondary lymphoid organs of OT1 transgenic mice using negative selection kit from Miltenyi and labeled with 1 uM Cell-Trace Violet.

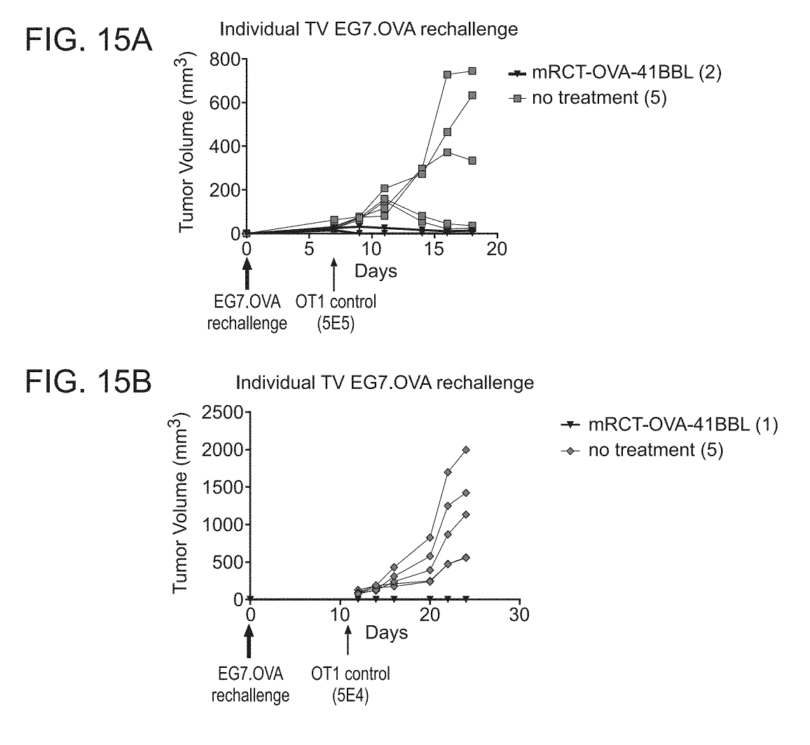

C57BL/6 mice were injected subcutaneously with 2E6 EG7.OVA tumor cells. When the tumor reached approximately 140 mm³, these mice were randomized into two groups of 7. These mice were then transferred with 2E6 OT1 T cells on day 1 post randomization and dosed with 1E9 mRCT or mRCT-OVA-4-1BBL on days 1, 4, and 8 post randomization. The mice cured with mRCT-OVA-4-1BBL (n=2) were re-challenged with 2E5 EG7.OVA, 37 days after primary tumor injection, side by side with age-matched naïve mice. Naïve mice also received 5E5 OT1 CD8 T cells transfer after 7 days of tumor injection. Tumor burden was measured every 2 to 3 days. The results of this experiment, as shown in FIG. 15A, demonstrate that mice that were previously treated with mRCT-OVA-4-1BBL were able to reject re-challenge of the EG7.OVA tumor cells, as evidenced by a reduction of EG7.OVA tumor volume over time as compared to that of the untreated mice.

In an independent experiment, C57BL/6 mice were injected subcutaneously with 2E6 EG7.OVA cells. When the tumor reached approximately 100 mm³, these mice were randomized into two groups of 12. They were then transferred with 5E5 OT1 T cells and dosed with 1E9 mRBC or mRBC-OVA-4-1BBL on days 0, 4, and 8 post randomization. The mouse cured with mRBC-OVA-4-1BBL(n=1) was re-challenged with 2E5 EG7.OVA, 35 days after primary tumor injection, side by side with age-matched naïve mice. Naïve mice also received 4E5 OT1 CD8 T cells transfer 11 days after tumor injection. Tumor burden was measured every 2 to 3 days. The results of this experiment, as shown in FIG. 15B, demonstrate that even upon being re-challenged with EG7.OVA tumor cells, mRCT-OVA-4-1BBL continued to activate tumor specific T cells (OT1), as evidenced by a reduction of EG7.OVA tumor volume over time as compared to that of the untreated mice.

Overall, these results demonstrate that mice that were previously cured of EG7.OVA tumors by mRCT treatment, maintain a memory response by preventing tumor growth upon being re-challenged with EG7.OVA tumor cells.

### Example 27: Mice Treated with Erythroid Cells Presenting MHCI (Ovalbumin) Only Demonstrate Antigen T Cell Specific Anergy Upon Re-Challenge with Antigen+Adjuvant

Murine erythroid cells were conjugated with MHCI presenting ovalbumin peptide using the click methodology, as described in Example 15. Briefly, mouse peripheral blood was filtered through a PAL de-leukocyte filter and labeled with 0.04 mM 6′ Azido-NHS-ester in pH8 PBS for 30 min at room temperature. Azido-labeled mRCT were incubated with 50 μM OVA-H2Kb-DBCO-thiolinker at room temperature for 1h and then further incubated at 4° C. overnight. CD8+ T cells were purified from secondary lymphoid organs of OT1 transgenic mice using negative selection kit from Miltenyi and labeled with 10 uM Cell-Trace Violet.

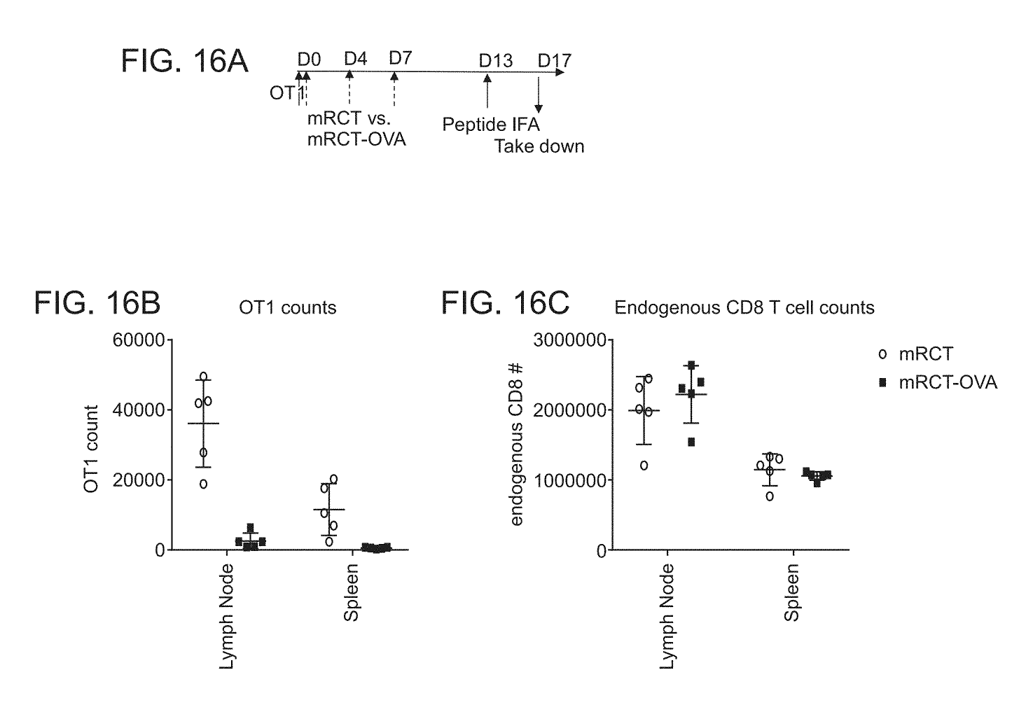

C57BL/6 mice were transferred with 2E6 OT1 T cells on day 0 and dosed with 1E9 mRCT or mRCT-OVA on days 0, 4, and 7. On day 13 mice were re-challenged with OVA peptide (SIINFEKL (SEQ ID NO: 721))+ Incomplete Freund's adjuvant (IFA) as shown in FIG. 16A. Mice treated with mRCT-OVA had lower OT1 cell counts upon OVA peptide re-challenge as compared to mice dosed only with mRCT in both spleen and lymph node as shown in FIG. 16B. The endogenous CD8+ T cell counts were not impacted by either treatment as demonstrated in FIG. 16C. Overall, these results demonstrate that mRCT-OVA was able to drive antigen specific T cell anergy or deletion, as shown by a reduction of OT1 count.

1. An artificial antigen presenting cell (aAPC), wherein the aAPC comprises an enucleated cell, wherein the enucleated cell comprises on the cell surface at least one exogenous antigenic polypeptide disclosed in Table 1 or Tables 14-24.

2-8. (canceled)

9. The aAPC of claim 1, wherein the aAPC further comprises on the cell surface an exogenous antigen-presenting polypeptide.

10. The aAPC of claim 9, wherein the exogenous antigen-presenting polypeptide is an MHC class I polypeptide, an MHC class I single chain fusion protein, an MHC class II polypeptide, or an MHC class II single chain fusion protein.

11-12. (canceled)

13. An artificial antigen presenting cell (aAPC), wherein the aAPC comprises an enucleated cell, wherein the enucleated cell comprises on the cell surface an exogenous antigen-presenting polypeptide and an exogenous antigenic polypeptide, wherein the exogenous antigen-presenting polypeptide is an MHC class I single chain fusion protein or an MHC class II single chain fusion protein.

14-22. (canceled)

23. The aAPC of claim 13, wherein the exogenous antigenic polypeptide is bound to the exogenous antigen-presenting polypeptide covalently.

24. The aAPC of claim 13, wherein the exogenous antigenic polypeptide is bound to the exogenous antigen-presenting polypeptide non-covalently.

25. The aAPC of claim 13, further comprising on the cell surface at least one exogenous costimulatory polypeptide and/or at least one exogenous cytokine polypeptide and/or at least one exogenous coinhibitory polypeptide.

26. The aAPC of claim 25, wherein the enucleated cell comprises at least one exogenous costimulatory polypeptide, and the at least one exogenous costimulatory polypeptide is selected from the group consisting of 4-1BBL, LIGHT, anti CD28, CD80, CD86, CD70, OX40L, GITRL, TIM4, SLAM, CD48, CD58, CD83, CD155, CD112, IL-15Rα fused to IL-15, IL-21, ICAM-1, a ligand for LFA-1, anti CD3, and a combination thereof.

27-28. (canceled)

29. The aAPC of claim 25, wherein the enucleated cell comprises at least one exogenous cytokine polypeptide, and the at least one exogenous cytokine polypeptide is selected from the group consisting of: IL2, IL15, 15Rα fused to IL-15, IL7, IL12, IL18, IL21, IL4, IL6, IL23, IL27, IL17, IL10, TGF-beta, IFN-gamma, IL-1 beta, GM-CSF, and IL-25.

30-31. (canceled)

32. The aAPC of claim 25, wherein the enucleated cell comprises at least one exogenous coinhibitory polypeptide, and the at least one exogenous co-inhibitory polypeptide is selected from the group consisting of the polypeptides disclosed in Table 7.

33-42. (canceled)

43. The aAPC of claim 1, further comprising on the cell surface an exogenous Treg cell expansion polypeptide.

44-54. (canceled)

55. The aAPC of claim 13, wherein the enucleated cell is an enucleated erythroid cell or a platelet.

56. A method of activating an antigen-specific T cell, the method comprising contacting the T cell with the aAPC of claim 13, thereby activating the antigen-specific T cell.

57. A method for inducing proliferation of a T cell expressing a receptor molecule, the method comprising contacting the T cell with the aAPC of claim 25, wherein the aAPC comprises an exogenous costimulatory polypeptide, and wherein the exogenous costimulatory polypeptide specifically binds with the receptor molecule, thereby inducing proliferation of said T cell.

58. A method of expanding a subset of a T cell population, the method comprising contacting a population of T cells comprising at least one T cell of the subset with an aAPC of claim 25, wherein the aAPC comprises an exogenous costimulatory polypeptide, and wherein the exogenous costimulatory polypeptide comprised on the surface of the aAPC specifically binds with a receptor molecule on the at least one T cell of the subset, and wherein binding of the exogenous costimulatory polypeptide to the receptor molecule induces proliferation of the at least one T cell of the subset, thereby expanding the subset of the T cell population.

59. A method of suppressing activity of a T cell, the method comprising contacting the T cell with the aAPC of claim 32, thereby suppressing activity of the T cell.

60. A method for activating a Treg cell, the method comprising contacting the Treg cell with the aAPC of claim 43, thereby activating the Treg cell.

61. A method of treating a subject in need of an altered immune response, the method comprising contacting a T cell of the subject with the aAPC of claim 1 or 13, thereby treating the subject in need of an altered immune response.

62. The method of claim 61, wherein the contacting is in vitro or in vivo.

63. (canceled)

64. A method of treating a subject in need of an altered immune response, the method comprising:
a) determining an expression profile of an antigen on a cell in the subject,
b) selecting an artificial antigen presenting cell (aAPC), wherein the aAPC is an engineered enucleated cell comprising on the cell surface an antigen-presenting polypeptide and at least one first exogenous antigenic polypeptide, and
c) administering the aAPC to the subject,
thereby treating the subject in need of the altered immune response.

65. A method of treating a subject in need of an altered immune response, the method comprising:
a) determining an HLA status of the subject,
b) selecting an artificial antigen presenting cell (aAPC) that is immunologically compatible with the subject, wherein the aAPC is an engineered enucleated cell comprising on the cell surface at least one first exogenous antigenic polypeptide and at least one antigen-presenting polypeptide, and
c) administering the aAPC to the subject,
thereby treating the subject in need of the altered immune response.

66. (canceled)

67. The method of claim 61, wherein the subject has cancer, an infectious disease, an autoimmune disease, or an allergic disease.

68-69. (canceled)

70. A method of inducing a T cell response to an antigen in a subject in need thereof, said method comprising:
obtaining a population of cells from the subject, wherein the population comprises a T cell,
contacting the population of cells with the aAPC of claim 13, wherein contacting the population of cells with the aAPC induces proliferation of an antigen-specific T cell that is specific for the at least one exogenous antigenic polypeptide, and
administering the antigen-specific T cell to the subject,
thereby inducing a T cell response to the antigen in the subject in need thereof.

71. (canceled)

72. A method of expanding a population of regulatory T (Treg) cells, the method comprising:
obtaining a population of cells from a subject, wherein the population comprises a Treg cell,
contacting the population with the aAPC of claim 43, wherein contacting the population with the aAPC induces proliferation of the Treg cell,
thereby expanding the population of Treg cells.

73-74. (canceled)

75. A method of making the aAPC of claim 1, the method comprising:
introducing an exogenous nucleic acid encoding the exogenous antigenic polypeptide into a nucleated cell; and
culturing the nucleated cell under conditions suitable for enucleation and for production of the exogenous antigenic polypeptide, thereby making an enucleated cell,
thereby making the aAPC.

76. (canceled)

77. A method of making the aAPC of claim 13, the method comprising:
introducing an exogenous nucleic acid encoding the exogenous antigenic polypeptide into a nucleated cell;
introducing an exogenous nucleic acid encoding the exogenous antigen-presenting polypeptide into the nucleated cell; and
culturing the nucleated cell under conditions suitable for enucleation and for production of the exogenous antigenic polypeptide and the exogenous antigen-presenting polypeptide, thereby making an enucleated cell,
thereby making the aAPC.

78-84. (canceled)
